SEQUENCE LISTING (65 sequences)

SEQ ID NO: 1 - protein, 309 aa, Homo sapiens
MSSTQFNKGPSYGLSAEVKNRLLSKYDPQKEAELRTWIEGLTGLSIGPDFQKGLKDGTILCTLMNKLQPGSVPKINRSMQNWHQLENLSNFIKAMVSYGMNPVDLFEANDLFESGNMTQVQVSLLALAGKAKTKGLQSGVDIGVKYSEKQERNFDDATMKAGQCVIGLQMGTNKCASQSGMTAYGTRRHLYDPKNHILPPMDHSTISLQMGTNKCASQVGMTAPGTRRHIYDTKLGTDKCDNSSMSLQMGYTQGANQSGQVFGLGRQIYDPKYCPQGTVADGAPSGTGDCPDPGEVPEYPPYYQEEAGY

SEQ ID NO: 2 - protein, 295 aa, Homo sapiens [sequence omitted]

SEQ ID NO: 3 - protein, 224 aa, Homo sapiens
MAETVDTSEMVNGATEQRTSSKESSPIPSPTSDRKAKTALPAQSAATLPARTQETPSAQMEGFLNRKHEWEAHNKKASSRSWHNVYCVINNQEMGFYKDAKTAASGIPYHSEVPVSLKEAVCEVALDYKKKEHVFKLRLNDGNEYLFQAKDDEEMNTWIQAISSAISSDKHEVSASTQSTPASSRAQTLPTSVVTITSESSPGKREKDKEKDKEKRFSLFGKKK

SEQ ID NO: 4 - protein, 585 aa, Homo sapiens
MPLPGYKVREIIQKLMLDGDRNKDGKISFDEFVYIFQEVKSSDIAKTFRKAINRKEGICALGGTSELSSEGTQHSYSEEEKYAFVNWINKALENDPDCRHVIPMNPNTDDLFKAVGDGIVLCKMINLSVPDTIDERAINKKKLTPFIIQENLNLALNSASAIGCHVVNIGAEDLRAGKPHLVLGLLWQIIKIGLFADIELSRNEALAALLRDGETLEELMKLSPEELLLRWANFHLENSGWQKINNFSADIKDSKAYFHLLNQIAPKGQKEGEPRIDINMSGFNETDDLKRAESMLQQADKLGCRQFVTPADVVSGNPKLNLAFVANLFNKYPALTKPENQDIDWTLLEGETREERTFRNWMNSLGVNPHVNHLYADLQDALVILQLYERIKVPVDWSKVNKPPYPKLGANMKKLENCNYAVELGKHPAKFSLVGIGGQDLNDGNQTLTLALVWQLMRRYTLNVLEDLGDGQKANDDIIVNWVNRTLSEAGKSTSIQSFKDKTISSSLAVVDLIDAIQPGCINYDLVKSGNLTEDDKHNNAKYAVSMARRIGARVYALPEDLVEVKPKMVMTVFACLMGRGMKRV

SEQ ID NO: 5 - protein, 304 aa, Homo sapiens
MTENGSHQLIVKARFNFKQTNEDELSVCKGDIIYVTRVEEGGWWEGTLNGRTGWFPSNYVREIKSSERPLSPKAVKGFETAPLTKNYYTVVLQNILDTEKEYAKELQSLLVTYLRPLQSNNNLSTVEVTSLLGNFEEVCTFQQTLCQALEECSKFPENQHKVGGCLLSLMPHFKSMYLAYCANHPSAVNVLTQHSDELEQFMENQGASSPGILILTTNLSKPFMRLEKYVTLLQELERHMEDTHPDHQDILKAIVAFKTLMGQCQDLRKRKQLELQILSEPIQAWEGEDIKNYCPMLSIRPRKL

SEQ ID NO: 6 - protein, 263 aa, Homo sapiens
MEKKKGEPRTRAEARPWVDEDLKDSSDLHQAEEDADEWQESEENVEHIPFSHNHYPEKEMVKRSQEFYELLNKRRSVRFISNEQVPMEVIDNVIRTAGTAPSGAHTEPWTFVVVKDPDVKHKIRKIIEEEEEINYMKRMGHRWVTDLKKLRTNWIKEYLDTAPILILIFKQVHGFAANGKKKVHYYNEISVSIACGILLAALQNAGLVTVTTTPLNCGPRLRVLLGRPAHEKLLMLLPVGYPSKEATVPDLKRKPLDQIMVTV

SEQ ID NO: 7 - protein, 567 aa, Homo sapiens
MADLANEEKPAIAPPVFVFQKDKGQKSPAEQKNLSDSGEEPRGEAEAPHHGTGHPESAGEHALEPPAPAGASASTPPPPAPEAQLPPFPRELAGRSAGGSSPEGGEDSDREDGNYCPPVKRERTSSLTQFPPSQSEERSSGFRLKPPTLIHGQAPSAGLPSQKPKEQQRSVLRPAVLQAPQPKALSQTVPSSGTNGVSLPADCTGAVPAASPDTAAWRSPSEAADEVCALEEKEPQKNESSNASEEEACEKKDPATQQAFVFGQNLRDRVKLINESVDEADMENAGHPSADTPTATNYFLQYISSSLENSTNSADASSNKFVFGQNMSERVLSPPKLNEVSSDANRENAAAESGSESSSQEATPEKESLAESAAAYTKATARKCLLEKVEVITGEEAESNVLQMQCKLFVFDKTSQSWVERGRGLLRLNDMASTDDGTLQSRLVMRTQGSLRLILNTKLWAQMQIDKASEKSIRITAMDTEDQGVKVFLISASSKDTGQLYAALHHRILALRSRVEQEQEAKMPAPEPGAAPSNEEDDSDDDDVLAPSGATAAGAGDEGDGQTTGST

SEQ ID NO: 8 - protein, 348 aa, Homo sapiens
MYTAIPQSGSPFPGSVQDPGLHVWRVEKLKPVPVAQENQGVFFSGDSYLVLHNGPEEVSHLHLWIGQQSSRDEQGACAVLAVHLNTLLGERPVQHREVQGNESDLFMSYFPRGLKYQEGGVESAFHKTSTGAPAAIKKLYQVKGKKNIRATERALNWDSFNTGDCFILDLGQNIFAWCGGKSNILERNKARDLALAIRDSERQGKAQVEIVTDGEEPAEMIQVLGPKPALKEGNPEEDLTADKANAQAAALYKVSDATGQMNLTKVADSSPFALELLISDDCFVLDNGLCGKIYIWKGRKANEKERQAALQVAEGFISRMQYAPNTQVEILPQGHESPIFKQFFKDWK

SEQ ID NO: 9 - protein, 201 aa, Homo sapiens
MGNVMEGKSVEELSSTECHQWYKKFMTECPSGQLTLYEFRQFFGLKNLSPSASQYVEQMFETFDFNKDGYIDFMEYVAALSLVLKGKVEQKLRWYFKLYDVDGNGCIDRDELLTIIQAIRAINPCSDTTMTAEEFTDTVFSKIDVNGDGELSLEEFIEGVQKDQMLLDTLTRSLDLTRIVRRLQNGEQDEEGADEAAEAAG

SEQ ID NO: 10 - protein, 320 aa, Homo sapiens
MDVIAEGDPGFKRTKGLMNRNATLSQVLREAKEKEEIRTSNEVTVETDKKTHYGLLFDEFQGLSHLEALEMLSQESEIKVKSILNSLSGEELETLKVELEQLKETFSLAEFCEEEEEEKKGDEDFTKDITELFSQLHVSSKPEKLARARNTAHEWIRKSLTKPLAENEEGEKQSEAENTEQVNKNSIEDIHAFAIRSLAELTACSIELFHKTAALVLHGRKQEVTAIERSQTLSQMTIVLCKELSSLSKEFTTCLTTAGVKEVADVLNPLITAVFLEASNSASYIQDAFQLLLPVLEISLIENKIESHRHELKEWGYFSI

SEQ ID NO: 11 - protein, 531 aa, Homo sapiens [sequence omitted]

SEQ ID NO: 12 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 13 - protein, 241 aa, Homo sapiens
MAEEQPQVELFVKAGSDGAKIGNCPFSQRLFMVLWLKGVTFNVTTVDTKRRTETVQKLCPGGQLPFLLYGTEVHTDTNKIEEFLEAVLCPPRYPKLAALNPESNTAGLDIFAKFSAYIKNSNPALNDNLEKGLLKALKVLDNYLTSPLPEEVDETSAEDEGVSQRKFLDGNELTLADCNLLPKLHIVQVVCKKYRGFTIPEAFRGVHRYLSNAYAREEFASTCPDDEEIELAYEQVAKALK

SEQ ID NO: 14 - protein, 251 aa, Homo sapiens
MTDSATANGDDRDPEIELFVKAGIDGESIGNCPFSQRLFMILWLKGVVFNVTTVDLKRKPADLHNLAPGTHPPFLTFNGDVKTDVNKIEEFLEETLTPEKYPKLAAKHRESNTAGIDIFSKFSAYIKNTKQQNNAALERGLTKALKKLDDYLNTPLPEEIDANTCGEDKGSRRKFLDGDELTLADCNLLPKLHVVKIVAKKYRNYDIPAEMTGLWRYLKNAYARDEFTNTCAADSEIELAYADVAKRLSRS

SEQ ID NO: 15 - protein, 492 aa, Homo sapiens
MDFLLGNPFSSPVGQRIEKATDGSLQSEDWALNMEICDIINETEEGPKDALRAVKKRIVGNKNFHEVMLALTVLETCVKNCGHRFHVLVASQDFVESVLVRTILPKNNPPTIVHDKVLNLIQSWADAFRSSPDLTGVVTIYEDLRRKGLEFPMTDLDMLSPIHTPQRTVFNSETQSGQDSVGTDSSQQEDSGQHAAPLPAPPILPGDTPIAPTPEQIGKLRSELEMVSGNVRVMSEMLTELVPTQAEPADLELLQELNRTCRAMQQRVLELIPQIANEQLTEELLIVNDNLNNVFLRHERFERFRTGQTTKAPSEAEPAADLIDMGPDPAATGNLSSQLAGMNLGSSSVRAGLQSLEASGRLEDEFDMFALTRGSSLADQRKEVKYEAPQATDGLAGALDARQQSTGAIPVTQACLMEDIEQWLSTDVGNDAEEPKGVTSEEFDKFLEERAKAADRLPNLSSPSAEGPPGPPSGPAPRKKTQEKDDDMLFAL

SEQ ID NO: 16 - protein, 204 aa, Homo sapiens
MFVLVEMVDTVRIPPWQFERKLNDSIAEELNKKLANKVVYNVGLCICLFDITKLEDAYVFPGDGASHTKVHFRCVVFHPFLDEILIGKIKGCSPEGVHVSLGFFDDILIPPESLQQPAKFDEAEQVWVWEYETEEGAHDLYMDTGEEIRFRVVDESFVDTSPTGPSSADATTSSEELPKKEAPYTLVGSISEPGLGLLSWWTSN

SEQ ID NO: 17 - protein, 157 aa, Homo sapiens
MQLTVKALQGRECSLQVPEDELVSTLKQLVSEKLNVPVRQQRLLFKGKALADGKRLSDYSIGPNSKLNLVVKPLEKVLLEEGEAQRLADSPPPQVWQLISKVLARHFSAADASRVLEQLQRDYERSLSRLTLDDIERLASRFLHPEVTETMEKGFSK

SEQ ID NO: 18 - protein, 309 aa, Homo sapiens
MAAAGAPDGMEEPGMDTEAETVATEAPARPVNCLEAEAAAGAAAEDSGAARGSLQPAPAQPPGDPAAQASVSNGEDAGGGAGRELVDLKIIWNKTKHDVKFPLDSTGSELKQKIHSITGLPPAMQKVMYKGLVPEDKTLREIKVTSGAKIMVVGSTINDVLAVNTPKDAAQQDAKAEENKKEPLCRQKQHRKVLDKGKPEDVMPSVKGAQERLPTVPLSGMYNKSGGKVRLTFKLEQDQLWIGTKERTEKLPMGSIKNVVSEPIEGHEDYHMMAFQLGPTEASYYWVYWVPTQYVDAIKDTVLGKWQYF

SEQ ID NO: 19 - protein, 186 aa, Homo sapiens
MQKVMYKGLVPEDKTLREIKVTSGAKIMVVGSTINDVLAVNTPKDAAQQDAKAEENKKEPLCRQKQHRKVLDKGKPEDVMPSVKGAQERLPTVPLSGMYNKSGGKVRLTFKLEQDQLWIGTKERTEKLPMGSIKNVVSEPIEGHEDYHMMAFQLGPTEASYYWVYWVPTQYVDAIKDTVLGKWQYF

SEQ ID NO: 20 - protein, 276 aa, Homo sapiens
MRLVILDNYDLASEWAAKYICNRIIQFKPGQDRYFTLGLPTGSTPLGCYKKLIEYHKNGHLSFKYVKTFNMDEYVGLPRNHPESYHSYMWNNFFKHIDIDPNNAHILDGNAADLQAECDAFENKIKEAGGIDLFVGGIGPDGHIAFNEPGSSLVSRTRLKTLAMDTILANAKYFDGDLSKVPTMALTVGVGTVMDAREVMILITGAHKAFALYKAIEEGVNHMWTVSAFQQHPRTIFVCDEDATLELRVKTVKYFKGLMHVHNKLVDPLFSMKDGN

SEQ ID NO: 21 - protein, 558 aa, Homo sapiens
MAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQRVRSGDWKGYTGKTITDVINIGIGGSDLGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 22 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 23 - protein, 529 aa, Homo sapiens [sequence omitted]

SEQ ID NO: 24 - protein, 377 aa, Homo sapiens
MNGPVSPKSKARPSSPSTSWHRPASPCPSPGPGHTLPPKPPSPRGTTASPKGRVRRKEEAKESPSAAGPEDKSQSKRRASNEKESAAPASPAPSPAPSPTPAPPQKEQPPAETPTAPAPPVTPSKPMAGTTDREEATRLLAEKRRQAREQREREEQERRLQAERDKRMREEQLAREAEARAEREAEARRREEQEAREKAQAEQEEQERLQKQKEEAEARSREEAERQRLEREKHFQQQEQERQERRKRLEEIMKRTRKSEVSETKKQDSKEANANGSSPEPVKAVEARSPGLQKEAVQKEEPIPQEPQWSLPSKELPASLVNGLQPLPARQENGFSTNGPSGDKSLSRTPETLLPFAEAEAFLKKAVVQSPQVTEVL

SEQ ID NO: 25 - protein, 227 aa, Homo sapiens
MAWKSGGASHSELIHNLRKNGIIKTDKVFEVMLATDRSHYAKCNPYMDSPQSIGFQATISAPHMHAYALELLFDQLHEGAKALDVGSGSGILTACSARMVGCTGKVIGIDHIKELVDDSVNNVRKDDPTLLSSGRVQLVVGDGRMGYAEEAPYDAIHVGAAAPVVPQALIDQLKPGGRLILPVGPAGGNQMLEQYDKLQDGSIKMKPLMGVIYVPLTDKEKQWSRWK

SEQ ID NO: 26 - protein, 184 aa, Homo sapiens
MAEPQPPSGGLTDEAALSCCSDADPSTKDFLLQQTMLRVKDPKKSLDFYTRVLGMTLIQKCDFPIMKFSLYFLAYEDKNDIPKEKDEKIAWALSRKATLELTHNWGTEDDETQSYHNGNSDPRGFGHIGIAVPDVYSACKRFEELGVKFVKKPDDGKMKGLAFIQDPDGYWIEILNPNKMATLM

SEQ ID NO: 27 - protein, 427 aa, Homo sapiens
MAATDIARQVGEGCRTVPLAGHVGFDSLPDQLVNKSVSQGFCFNILCVGETGLGKSTLMDTLFNTKFEGGPATHTQPGVQLQSNTYDLQESNVRLKLTIVSTVGFGDQINKEDSYKPIVEFIDAQFEAYLQEELKIRRVLHTYHDSRIHVCLYFIAPTGHSLKSLDLVTMKKLDSKVNIIPIIAKADAISKSELTKFKIKITSELVSNGVQIYQFPTDDESVAEINGTMNAHLPFAVVGSTEELKIGNKMMRARQYPWGTVQVENEAHCDFVKLREMLIRVNMEDLREQTHTRHYELYRRCKLEEMGFKDTDPDSKPFSLQETYEAKRNEFLGELQKKEEEMRQMFVQRVKEKEAELKEAEKELHEKFDRLKKLHQDEKKKLEDKKKSLDDEVNAFKQRKTAAELLQSQGSQAGGSQTLKRDKEKKN

SEQ ID NO: 28 - protein, 528 aa, Homo sapiens
MGDAPSPEEKLHLITRNLQEVLGEEKLKEILKERELKIYWGTATTGKPHVAYFVPMSKIADFLKAGCEVTILFADLHAYLDNMKAPWELLELRVSYYENVIKAMLESIGVPLEKLKFIKGTDYQLSKEYTLDVYRLSSVVTQHDSKKAGAEVVKQVEHPLLSGLLYPGLQALDEEYLKVDAQFGGIDQRKIFTFAEKYLPALGYSKRVHLMNPMVPGLTGSKMSSSEEESKIDLLDRKEDVKKKLKKAFCEPGNVENNGVLSFIKHVLFPLKSEFVILRDEKWGGNKTYTAYVDLEKDFAAEVVHPGDLKNSVEVALNKLLDPIREKFNTPALKKLASAAYPDPSKQKPMAKGPAKNSEPEEVIPSRLDIRVGKIITVEKHPDADSLYVEKIDVGEAEPRTVVSGLVQFVPKEELQDRLVVVLCNLKPQKMRGVESQGMLLCASIEGINRQVEPLDPPAGSAPGEHVFVKGYEKGQPDEELKPKKKVFEKLQADFKISEECIAQWKQTNFMTKLGSISCKSLKGGNIS

SEQ ID NO: 29 - protein, 190 aa, Homo sapiens
MDHSHHMGMSYMDSNSTMQPSHHHPTTSASHSHGGGDSSMMMMPMTFYFGFKNVELLFSGLVINTAGEMAGAFVAVFLLAMFYEGLKIARESLLRKSQVSIRYNSMPVPGPNGTILMETHNTVGQQMLSFPHLLQTVLHIIQVVISYFLMLIFMTYNGYLCIAVAAGAGTGYFLFSWKKAVVVDITEHCH

SEQ ID NO: 30 - protein, 117 aa, Homo sapiens
MRAEKRKKNAPKEASRLKSILKLDGDVLMKDAQEIATVVVPKPKHCQEKMQCEVKDEKDDMKMETDIKRNKKTLLDQHGQYPIWMNQRQRKRLKAKREKRKGKSKAKAVKVAKGLAW

SEQ ID NO: 31 - protein, 217 aa, Homo sapiens
MPVRTECPPPAGASAASAASLIPPPPINTQQPGVATSLLYSGSKFRGHQKSKGNSYDVEVVLQHVDTGNSYLCGYLKIKGLTEEYPTLTTFFEGEIISKKHPFLTRKWDADEDVDRKHWGKFLAFYQYAKSFNSDDFDYEELKNGDYVFMRWKEQFLVPDHTIKDISGASFAGFYYICFQKSAASIEGYYYHRSSEWYQSLNLTHVPEHSAPIYEFR

SEQ ID NO: 32 - protein, 299 aa, Homo sapiens
MVDKKLVVVFGGTGAQGGSVARTLLEDGTFKVRVVTRNPRKKAAKELRLQGAEVVQGDQDDQVIMELALNGAYATFIVTNYWESCSQEQEVKQGKLLADLARRLGLHYVVYSGLENIKKLTAGRLAAAHFDGKGEVEEYFRDIGVPMTSVRLPCYFENLLSHFLPQKAPDGKSYLLSLPTGDVPMDGMSVSDLGPVVLSLLKMPEKYVGQNIGLSTCRHTAEEYAALLTKHTRKVVHDAKMTPEDYEKLGFPGARDLANMFRFYALRPDRDIELTLRLNPKALTLDQWLEQHKGDFNLL

SEQ ID NO: 33 - protein, 312 aa, Homo sapiens [sequence omitted]

SEQ ID NO: 34 - protein, 149 aa, Homo sapiens [sequence omitted]

SEQ ID NO: 35 - protein, 284 aa, Homo sapiens [sequence omitted]

SEQ ID NO: 36 - protein, 368 aa, Homo sapiens
MVCAGDELTANELCRLGALDILEEVNVSGTRKNKFSEAAYNKLLNNNLSLKYSQTGYLSSSNIINDGFYDYGRINPGTKLLPLKELCLQEPSDLRAVLLINSKSYVSPPSSMEDKSDVGYGRSISSSSSLRRSSKEKNKKNSYHFSAGFGSPIEDKSEPASGRNTVLSKSATKEKGWRKSKGKKEEEKVKEEEEVMVVPKFVGEGSSDKEWCPPSDPDFSMYVYEVTKSILPITNIKEQIEDLAKYVAEKMGGKIPKEKLPDFSWELHISELKFQLKSNVIPIGHVKKGIFYHRALLFKALADRIGIGCSLVRGEYGRAWNEVMLQNDSRKGVIGGLPAPEMYVIDLMFHPGGLMKLRSREADLYRFI

SEQ ID NO: 37 - protein, 367 aa, Homo sapiens [sequence omitted]

SEQ ID NO: 38 - protein, 293 aa, Homo sapiens
MLSSNGASKVANSEAMILDKNLESVNSPIEKSSVNYEPSNPSEKGSKKINLSSDQNKSVSESNNDDVMLISVESPNLTTPTTSNPTDTRKITSGNSSNSPNAEVMAVQKKLDSIIDLTKEGLSNCNTESPVSPLESHSKAASNSKETTPLAQNAVQVPESFEHLPPLPEPPAPLPELVDKTRDTLPPQKPELKVKRVFRPNGIALTWNITKINPKCAPVESYHLFLCHENSNNKLIWKKIGEIKALPLPMACTLSQFLASNRYYFTVQSKDIFGRYGPFCDIKSIPGFSENLT

SEQ ID NO: 39 - protein, 232 aa, Homo sapiens [sequence omitted]

SEQ ID NO: 40 - protein, 300 aa, Homo sapiens
MSGSNGSKENSHNKARTSPYPGSKVERSQVPNEKVGWLVEWQDYKPVEYTAASVLAGPRWADPQISESNFSPKFNEKDGHVERKSKNGLYEIENGRPRNPAGRTGLVGRGLLGRWGPNHAADPIITRWKRDSSGNKIMHPVSGKHILQFVAIKRKDCGEWAIPGGMVDPGEKISATLKREFGEEALNSLQKTSAEKREIEEKLHKLFSQDHLVIYKGYVDDPRNTDNAWMETEAVNYHDETGEIMDNLMLEAGDDAGKVKWVDINDKLKLYASHSQFIKLVAEKRDAHWSEDSEADCHAL

SEQ ID NO: 41 - protein, 107 aa, Homo sapiens [sequence omitted]

SEQ ID NO: 42 - protein, 219 aa, Homo sapiens
MADKAKPAKAANRTPPKSPGDPSKDRAAKRLSLESEGAGEGAAASPELSALEEAFRRFAVHGDARATGREMHGKNWSKLCKDCQVIDGRNVTVTDVDIVFSKIKGKSCRTITFEQFQEALEELAKKRFKDKSSEEAVREVHRLIEGKAPIISGVTKAISSPTVSRLTDTTKFTGSHKERFDPSGKGKGKAGRVDLVDESGYVSGYKHAGTYDQKVQGGK

SEQ ID NO: 43 - protein, 134 aa, Homo sapiens
MPPRRSIVEVKVLDVQKRRVPNKHYVYIIRVTWSSGSTEAIYRRYSKFFDLQMQMLDKFPMEGGQKDPKQRIIPFLPGKILFRRSHIRDVAVKRLIPIDEYCKALIQLPPYISQCDEVLQFFETRPEDLNPPKE

SEQ ID NO: 44 - protein, 175 aa, Homo sapiens
MATATNELGQATCAASLTVRPGGSTSPFSSPITSDEEYLSPPEEFPEPGETWPRTPTMKPSPSQNRRSSDTGSKAPPTFKVSLMDQSVREGQDVIMSIRVQGEPKPVVSWLRNRQPVRPDQRRFAEEAEGGLCRLRILAAERGDAGFYTCKAVNEYGARQCEARLEVRGEYLISP

SEQ ID NO: 45 - protein, 150 aa, Homo sapiens
MLVSQVLPPAPGLALPLKPETAISVPEGGLPVAPSPALPEAHALGTLSAQQPPPAAATTSSTSLPFSPDSPGLLPNFPAPPPEGLMLSPAAVPVWSAGLELSAGTEGLLEAEKGLGTQAPHTMLRLPDPDPEGLLLGATAGGEVDEGLEA

SEQ ID NO: 46 - protein, 337 aa, Homo sapiens
MSSSPVKRQRMESALDQLKQFTTVVADTGDFHAIDEYKPQDATTNPSLILAAAQMPAYQELVEEAIAYGRKLGGSQEDQIKNAIDKLFVLFGAEILKKIPGRVSTEVDARLSFDKDAMVARARRLIELYKEAGISKDRILIKLSSTWEGIQAGKELEEQHGIHCNMTLLFSFAQAVACAEAGVTLISPFVGRILDWHVANTDKKSYEPLEDPGVKSVTKIYNYYKKFSYKTIVMGASFRNTGEIKALAGCDFLTISPKLLGELLQDNAKLVPVLSAKAAQASDLEKIHLDEKSFRWLHNEDQMAVEKLSDGIRKFAADAVKLERMLTERMFNAENGK

SEQ ID NO: 47 - protein, 127 aa, Homo sapiens [sequence omitted]

SEQ ID NO: 48 - protein, 233 aa, Homo sapiens
MRPQQLHATEITSSGFRLAWPPLLTADSGYYVLELVPSAQPGAARRQQLPGNATDWIWAGLDPDTDYDVALVPESNVRLLRPQILRVRTRPGEAGPGASGPESGAGPAPTQLAALPAPEEAGPERIVISHARPRSLRVSWAPALGSAAALGYHVQFGPLRGGEAQRVEVPAGRNCTTLQGLAPGTAYLVTVTAAFRSGRESALSAKACTPDGPRPRPRPVPRAPTPGTASREP

SEQ ID NO: 49 - protein, 333 aa, Homo sapiens [sequence omitted]

SEQ ID NO: 50 - protein, 149 aa, Homo sapiens
MESRGKSASSPKPDTKVPQVTTEAKVPPAADGKAPLTKPSKKEAPAEKQQPPAAPTTAPAKKTSAKADPALLNNHSNLKPAPTVPSSPDATPEPKGPGDGAEEDEAASGGPGGRGPWSCENFNPLLVAGGVTVAAIALILGVAFLVRKK

SEQ ID NO: 51 - protein, 262 aa, Homo sapiens
MSRLSWGYREHNGPIHWKEFFPIADGDQQSPIEIKTKEVKYDSSLRPLSIKYDPSSAKIISNSGHSFNVDFDDTENKSVLRGGPLTGSYRLRQVHLHWGSADDHGSEHIVDGVSYAAELHVVHWNSDKYPSFVEAAHEPDGLAVLGVFLQIGEPNSQLQKITDTLDSIKEKGKQTRFTNFDLLSLLPPSWDYWTYPGSLTVPPLLESVTWIVLKQPINISSQQLAKFRSLLCTAEGEAAAFLVSNHRPPQPLKGRKVRASFH

SEQ ID NO: 52 - protein, 437 aa, Homo sapiens
MAAGTLYTYPENWRAFKALIAAQYSGAQVRVLSAPPHFHFGQTNRTPEFLRKFPAGKVPAFEGDDGFCVFESNAIAYYVSNEELRGSTPEAAAQVVQWVSFADSDIVPPASTWVFPTLGIMHHNKQATENAKEEVRRILGLLDAYLKTRTFLVGERVTLADITVVCTLLWLYKQVLEPSFRQAFPNTNRWFLTCINQPQFRAVLGEVKLCEKMAQFDAKKFAETQPKKDTPRKEKGSREEKQKPQAERKEEEKATAPAPEEEMDECEQALAAEPKAKDPFAHLPKSTFVLDEFKRKYSNEDTLSVALPYFWEHFDKDGWSLWYSEYRFPEELTQTFMSCNLITGMFQRLDKLRKNAFASVILFGTNNSSSISGVWVFRGQELAFPLSPDWQVDYESYTWRKLDPGSEETQTLVREYFSWEGAFQHVGKAFNQGKIFK

SEQ ID NO: 53 - protein, 221 aa, Homo sapiens
MMRWNFSPEDLSSIFRNNSTLPKITVKNVDIEFTIPTAVTIEVEPSPVQQDNPPISSEQADFSLAQPDSPSLPLESPEESESSAQQEATAQTPNPPKEVEPSPVQQEFPAEPTEPAKEVEPSATQQEASGHPLKSTKEVNPPPKQEIPAQPSEPPEKVELSPVLQQAPTQLLEPLKKVECSPVQQAVPAQSSEPSIVVEPSPVQQIAHLCLQSSLRKWNPL

SEQ ID NO: 54 - protein, 250 aa, Homo sapiens
MAEGGASKGGGEEPGKLPEPAEEESQVLRGTGHCKWFNVRMGFGFISMINREGSPLDIPVDVFVHQSKLFMEGFRSLKEGEPVEFTFKKSSKGLESIRVTGPGGSPCLGSERRPKGKTLQKRKPKGDRCYNCGGLDHHAKECSLPPQPKKCHYCQSIMHMVANCPHKNVAQPPASSQGRQEAESQPCTSTLPREVGGGHGCTSPPFPQEARAEISERSGRSPQEASSTKSSIAPEEQSKKGPSVQKRKKT

SEQ ID NO: 55 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSGNGKTSKKITIADCGQLE

SEQ ID NO: 56 - protein, 126 aa, Homo sapiens
MPEPSKSAPAPKKGSKKAVTKAQKKDGKKRKRSRKESYSVYVYKVLKQVHPDTGISSKAMGIMNSFVNDIFERIAGEASRLAHYNKRSTITSREIKTAVRLLLPGELAKHAVSEGTKAVTKYTSSK

SEQ ID NO: 57 - protein, 318 aa, Homo sapiens [sequence omitted]

SEQ ID NO: 58 - protein, 266 aa, Homo sapiens
MDLSVPHPQPAAMAAYKLVLIRHGESAWNLENRFSGWYDADLSPAGHEEAKRGGQALRDAGYEFDICFTSVQKRAIRTLWTVLDAIDQMWLPVVRTWRLNERHYGGLTGLNKAETAAKHGEAQVKIWRRSYDVPPPPMEPDHPFYSNISKDRRYADLTEDQLPSCESLKDTIARALPFWNEEIVPQIKEGKRVLIAAHGNSLRGIVKHLEGLSEEAIMELNLPTGIPIVYELDKNLKPIKPMQFLGDEETVRKAMEAVAAQGKAKK

SEQ ID NO: 59 - protein, 240 aa, Homo sapiens [sequence omitted]

SEQ ID NO: 60 - protein, 234 aa, Homo sapiens
MKNVEPSQRDKGYLIHVGGLCPSVSEADLRSHFQKYQVSEISIYDSTNYRYASLAFTKNSDAKIAVKEMNGIEINGKSVNVWPVKILGEYTSPLSSKNGNRISSNNLEKSTNKQIHSEFSISRLPRTRPRQLGSEQDSEVFPSDQGVKKNCKQIESAKLLPDTPVQFIPPNTLNLRSFTKIIKRLAELHPEVSRDHIINALQEVRIRHKGFLNGLSITTIVEMTSSLLKNSASS

SEQ ID NO: 61 - protein, 190 aa, Homo sapiens
MNEFFSVDDNNEEEEDVEMKEDSDENGPEEKQSVEEMEEQSQDADGVNTVTVPGPASEEAVEDCKDEDFAKDENITKGGEVTDHSVRDQDHPDGQENDSTKNEIKIETESQSSYMETEELSSNQEDAVIVEQPEVIPLTEDQEEKEGEKAPGEDTPRMPGKSEGSSDLENTPGPDAGAQDEAKEQRNGTK

SEQ ID NO: 62 - protein, 147 aa, Homo sapiens
MRSESPGKWGNSPGLHHSSTGKSPASSLPGRGVPELRVTPTAPSAEGGRKTAPSHGSAHSASPPASLSATDPWPLAAQTLSTPRRTNTTLMGPAAMSTPAAGAPSASTDPAQRIVVTGRGPTPRGHVAHAQLAQPTARTKSKVSFRE

SEQ ID NO: 63 - protein, 104 aa, Homo sapiens
MEDPTLYIVERPLPGYPDAEAPEPSSAGAQAAEEPSGAGSEELIKSDQVNGVLVLSLLDKIIGAVDQIQLTQAQLEERQAEMEGAVQSIQGELSKLGKAQLPPS

SEQ ID NO: 64 - DNA, 2858 bp, Homo sapiens
  CDS (606)..(2363)
agattgctcttctgggcgtgggagaaggttctgtctatcagtgctgcgagaaaggaaagaaacaagtttgctctcagcggatctttaaatggatgagatggctaccactcagatttccaaagatgagcttgatgaactcaaagaggcctttgcaaaagttgtggagacggagtttcaccgtgttagccaggatggtctcgatctcctgacctcgtgatccgcccgtctcggcctcccaaagtgctgggattacagacgtgagccaccgcgcctggcctacacacatgtatttttaaaacgagagttgcagcaggggaaaaatgatggccaaactgctggaaattttgagtcagaaaaggactgataaacatttttgattgcctggtctcaccctctaaacttgtccagccttctgctgtacttgacctccatctttggaaatccactagtacagtgaattctaaagcagcaacctccagtctacccttagctggaactcattaaactgcctcttatatttgctgcagtgagctacctcaaaagatctcaacagcaacggattcatttgtgactatgaacttcatgagctcttcaaggaagctaatatgccattaccaggatataaagtgagagaaattattcagaaactcatgctggatggtgacaggaataaagatgggaaaataagttttgacgaatttgtttatatttttcaagaggtaaaaagtagtgatattgccaagaccttccgcaaagcaatcaacaggaaagaaggtatttgtgctctgggtggaacttcagagttgtccagcgaaggaacacagcattcttactcagaggaagaaaaatatgcttttgttaactggataaacaaagctttggaaaatgatcctgattgtagacatgttataccaatgaaccctaacaccgatgacctgttcaaagctgttggtgatggaattgtgctttgtaaaatgattaacctttcagttcctgataccattgatgaaagagcaatcaacaagaagaaacttacacccttcatcattcaggaaaacttgaacttggcactgaactctgcttctgccattgggtgtcatgttgtgaacattggtgcagaagatttgagggctgggaaacctcatctggttttgggactgctttggcagatcattaagatcggtttgttcgctgacattgaattaagcaggaatgaagccttggctgctttactccgagatggtgagactttggaggaacttatgaaattgtctccagaagagcttctgcttagatgggcaaactttcatttggaaaactcgggctggcaaaaaattaacaactttagtgctgacatcaaggattccaaagcctatttccatcttctcaatcaaatcgcaccaaaaggacaaaaggaaggtgaaccacggatagatattaacatgtcaggtttcaatgaaacagatgatttgaagagagctgagagtatgcttcaacaagcagataaattaggttgcagacagtttgttacccctgctgatgttgtcagtggaaaccccaaactcaacttagctttcgtggctaacctgtttaataaatacccagcactaactaagccagagaaccaggatattgactggactctattagaaggagaaactcgtgaagaaagaaccttccgtaactggatgaactctcttggtgtcaatcctcacgtaaaccatctctatgctgacctgcaagatgccctggtaatcttacagttatatgaacgaattaaagttcctgttgactggagtaaggttaataaacctccatacccgaaactgggagccaacatgaaaaagctagaaaactgcaactatgctgttgaattagggaagcatcctgctaaattctccctggttggcattggagggcaagacctgaatgatgggaaccaaaccctgactttagctttagtctggcagctgatgagaagatataccctcaatgtcctggaagatcttggagatggtcagaaagccaatgacgacatcattgtgaactgggtgaacagaacgttgagtgaagctggaaaatcaacttccattcagagttttaaggacaagacgatcagctccagtttggcagttgtggatttaattgatgccatccagccaggctgtataaactatgaccttgtgaagagtggcaatctaacagaagatgacaagcacaataatgccaagtatgcagtgtcaatggctagaagaatcggagccagagtgtatgctctccctgaagaccttgtggaagtaaagcccaagatggtcatgactgtgtttgcatgtttgatgggcaggggaatgaagagagtgtaaaataaccaatctgaataaaacagccatgctcccaggtgcatgattcgcaggtcagctatttccaggtgaagtgcttatggcttaaggaactcttggccattcaaaggacttttcattttgattaacaggactagcttatcatgagagccctcaggggaaagggtttaagaaaaacaactcctctttcccatagtcagggttgaatttgtcaggcacgcctgaaatgtgctcatagccaaaacattttactctctcctcctagaatgctgcccttgacatttcccattgctgtatgttatttcttgctctgttatcttttgccctcttagaatgtccctctcttgggacttgcttagatgatgggatatgaatattattagacagtaattttgctttccatccagtatgctagttcttattcgagaactatggtcagagcgtatttggatatgagtatcctttgcttatctttgtagtactgaaaatttgccgaag

SEQ ID NO: 65 - DNA, 1523 bp, Homo sapiens
  CDS (571)..(1485)
agactctgacttccctttagattaaaaaacaatccctgggctggagaggttgcgggcgcggcacagagtttggagacgggcgccccctcctcgcccgcgtccaggcgcaaagttgcagccgcccctccccgctggggcgagcctagcccagctctccagtttcgccctggtggcggccgcgctcagggcagggtcccagctccggctgggttgatctgttcccgcagccttggcacaggcttgcccgcccgctcgccggccccgtgactcgggagggcgaacggcttccaggaggcggcggcggcgcggctggagtgagggagcagctggaggacaggcgggcagcggccgagccagatatttgatcctgatgacctttattcaggggtcaatttctccaaggtactgagtactcttttagctgtcaacaaagcaacagaagatcagctatcagaaagaccatgtggacgttcctcttctcttagtgctgctaatacttctcagacaaacccacagggagcagtttctagcacagtttcagggctgcaaaggcagtcaaagacagtggagatgacggaaaatggaagtcatcagttgatagtaaaagcaagattcaactttaagcagactaatgaggatgaactgtcagtttgtaagggggacatcatttacgtcacacgagttgaagaaggaggctggtgggaaggcacattaaatgggagaacaggctggttccccagtaattatgtccgtgaaattaaatccagtgagagacctctctccccaaaagccgtcaaaggatttgaaactgctccacttaccaagaattattatactgtggtgttacagaacatcctggacactgaaaaagaatatgctaaagaacttcagtctcttcttgttacttacttaagacccctgcagtccaataacaatctgagtactgtggaggttacatctttactgggaaacttcgaggaagtatgcacatttcaacagacactctgccaagccttggaagaatgttcaaagtttccagaaaaccagcacaaagtaggaggttgtctactgagtctcatgcctcattttaaatctatgtatctggcttactgtgcaaaccatccttcagctgtaaatgtgctcactcagcacagtgatgagttggaacaattcatggaaaatcaaggtgcatcgagcccaggtatcctcattttaacaacaaacctcagcaaaccattcatgcgactggagaaatatgttactctcttgcaagagttagaacggcatatggaggatactcatccagatcatcaggatattctgaaagcaatcgtagcattcaaaactctcatggggcaatgtcaagatctgaggaagagaaaacagctggagttacagatactgtccgaacctattcaggcatgggaaggagaagatattaaaaattactgccctatgttgtctataagaccaagaaaactgtagtacccttattccttttgtgtcatgtaaattgtaactca

(19) United States
(12) Patent Application Publication
(10) Pub. No.: US 2011/0269141 A1
(43) Pub. Date: Nov. 3, 2011

(54) TARGET PROTEIN AND TARGET GENE FOR DRUG DISCOVERY, AND SCREENING METHOD

(75) Inventors: Katsuhisa Murayama, Osaka (JP); Tadakazu Yamauchi, Shizuoka (JP); Kouichi Tsuchiya, Tokyo (JP); Kazuo Komiya, Osaka (JP); Morikazu Kito, Kanagawa (JP); Yuko Isono, Kanagawa (JP); Noriyuki Inomata, Kanagawa (JP); Yorimasa Suwa, Tokyo (JP); Ai Wakamatsu, Tokyo (JP); Junichi Yamamoto, Chiba (JP); Takao Isogai, Ibaraki (JP)

(73) Assignee: REVERSE PROTEOMICS RESEARCH INSTITUTE CO., LTD., Tokyo (JP)

(21) Appl. No.: 12/528,190

(22) PCT Filed: Feb. 20, 2008
(86) PCT No.: PCT/JP2008/053345

§ 371 Date: Aug. 21, 2009

(30) Foreign Application Priority Data
Feb. 21, 2007 (JP) 2007-040541

(51) Int. Cl. C12Q 1/68; C07H 21/04; G01N 33/50; C07K 16/00; C07K 14/00; G01N 33/68; C07H 21/02; C12N 15/63

(52) U.S. Cl. 435/6.13; 536/23.1; 536/24.5; 435/320.1; 530/387.1; 530/350; 530/395; 436/86; 436/94

(57) ABSTRACT

The problems of the present invention are to provide target proteins and target genes for bioactive substances such as drugs, and means that enable the development of novel bioactive substances using the same. The present invention provides target proteins and target genes for bioactive substances; screening methods for substances capable of regulating bioactivities; bioactivity regulators; a bioactive substance derivative production method; a complex comprising a bioactive substance and a target protein, and a method of producing the complex; and kits comprising a bioactive substance or a salt thereof; determination methods for the onset or risk of onset of a specified disease or condition, determination methods for susceptibility to a bioactive substance, and determination kits used for the determination methods, and the like.

### TECHNICAL FIELD

The present invention relates to target proteins and target genes that are useful for the development of bioactive substances, for example, drug discovery; a screening method for a bioactive substance and the substance obtained by the screening method; a bioactivity regulator; a bioactive substance derivative and a method of producing the derivative; and a complex comprising a bioactive substance and a target protein therefor and a method of producing the complex, and the like.

### BACKGROUND OF THE INVENTION

Traditionally, the success rate of new drug research and development is quite low, with only one or two of about 100 research projects ending successfully with the launch of a new drug (D. Brown and G. Superti-Furga, Drug Discovery Today, December, 2003). This is mostly because of premature termination of the development due to a problem with the economy, safety or efficacy of the new drug candidate compound (Dimasi, Clin. Pharmacol. Ther., 69, 297-307, 2001).

Pharmaceutical companies are spending 10 to 20% of their sales on R&D activities; it is of paramount importance to efficiently spend R&D budgets for pharmaceutical companies to be highly competitive. Furthermore, because about 80% of R&D expenditures are spent for costly clinical studies in the developmental stage, it is most critical to select appropriate candidate compounds in the initial stage prior to progress to the developmental stage.

In recent years, on the other, the genome sequences of a variety of organisms have been elucidated and analyzed at the global level. For the human genome, in particular, a worldwide cooperative research project was implemented, and completion of analysis of all sequences thereof was announced in April 2003. As a result, it is becoming possible to analyze complex biological phenomena in the context of the functions and control of all genes, or networks of gene-gene, protein-protein, cell-cell, and individual-individual interactions. The genome information thus obtained has been significantly revolutionizing a number of industries, including drug development, as well as in academic sectors.

For example, it has been reported that there are about 480 kinds of target proteins for drugs having been in common use to date, and that these target proteins are limited to membrane receptors, enzymes, ion channels, or nuclear receptors and the like (J. Drews, Science, 297, 1960-1964, 2000). Meanwhile, target protein search based on genome information has discovered an extremely large number of target proteins, including novel proteins not covered in the conventional range of target proteins one after another, which are estimated to total about 1,500 kinds (A. L. Hopkins & C. R. Groom, Nature Reviews; Drug Discovery, 1, 727-730, 2002).

However, despite the fact that the research and development expenditures spent by pharmaceutical companies are increasing due to rises in infrastructuring costs for coping with vast amounts of data like genome information and clinical developmental costs, the number of new drugs approved is tending to decrease on the contrary (S. Franz & A. Smith, Nature Reviews; Drug Discovery, February, 2003). This shows that the above-described genome information is actually not efficiently utilized.

As a means for overcoming these circumstances, Nagashima et al. invented “Method, System, Apparatus, and Device for Discovering and Preparing Chemical for Medical and Other Uses” and filed a patent application for that invention (JP 2004-509406 A).

Disclosed in that patent application are methods, systems, databases, user interfaces, software, media, and services that are useful for the evaluation of compound-protein interactions, and are also useful for the utilization of the information resulting from such an evaluation intended to discover compounds in medical and other areas. Furthermore, it is intended to produce a very large pool of novel target proteins for drug discovery, novel methods for designing novel drugs, and a pool of small substances for therapeutic purposes that are virtually synthesized as having been inconceivable in the past.

Specifically, disclosed in that patent application were a method of identifying a protein or partial protein that is appropriate as a novel drug discovery target, which comprises the following steps:
(i) a step for selecting a plurality of proteins or partial proteins showing desired affinity and specificity for a selected target compound;
(ii) a step for identifying the structure and function of the protein or the partial protein; and
(iii) a step for selecting a single protein or single partial protein having a desired function, and a method of discovering a drug, which comprises the following steps:
(i) a step for investigating the chemical structure of the target compound selected using the above-described method; and
(ii) a step for chemically modifying the structure of the selected target compound to optimize the affinity and specificity of the modified compound for the protein or the partial protein, which is appropriate as a novel drug target.

Furthermore, another feature of the method disclosed in that patent application resides in that the selected target compound is a compound approved for medical use.

Conventional drugs that have been used to date include many drugs for which target proteins are unknown, or for which target proteins are known but not all of whose pharmacological effects and adverse effects can be explained by mechanisms mediated by the proteins.

Typically, aspirin, one of the drugs that have longest been used, may be mentioned. When aspirin was launched in the market for the first time more than 100 years ago, the mechanism for its anti-inflammatory action was unclear. About 70 years later, aspirin was found to have cyclooxygenase (COX) inhibitory action. Still 20 years later, it was demonstrated that COX occurred in two subtypes: COX-1 and COX-2, that the primary pharmacological effect of aspirin was based on COX-2 inhibition, and that COX-1 inhibitory action was the cause of adverse effects such as gastrointestinal disorders. However, not all the target proteins for aspirin have been elucidated. In recent years, aspirin has been shown to exhibit anticancer action and antidementic action in clinical settings, but these pharmacological effects cannot be explained by COX inhibition. On the other, recent years have seen many papers reporting that aspirin acts on transcription factors such as IKKβ and on nuclear receptors such as PPAR-γ, but the association of these and the various pharmacological effects of aspirin remains unclear.

For these reasons, elucidating target proteins for traditionally used drugs can be said to be a very effective approach to discovering novel drug discovery target proteins.

Hirayama, one of the inventors of the above-described published patent, and others generated a database integrating the structural and physical property data on about 1,500 kinds of drugs commercially available in Japan, and found that existing pharmaceutical compounds share structural features (I. Fujii et al., Chem-Bio Informatics Journal, 1, 18-22, 2001). Drugs that have been commonly used to date can be described as excellent in that they have cleared the issues of localization in the body and safety in their developmental processes. Searching novel target proteins with these existing drugs as probes, and selecting novel new drug candidate compounds on the basis of their structures is thought to be a highly reasonable and efficient approach.

A second problem arises concerning how to make use of the genome information during the search for novel target proteins. Solely determining the genome sequence is not sufficient to ensure the elucidation of the functions of all genes and the discovery of drug discovery target proteins. It is estimated that in humans, about 30,000 to 40,000 kinds of genes are present; taking into consideration variants from alternative splicing, there are reportedly more than 100,000 kinds of mRNA. It is important, therefore, that out of the vast amount of new genes revealed from the genome sequence, those having useful functions in industrial applications, including drug development, should be efficiently selected and identified.

In the genome sequences of eukaryotic organisms, each gene is divided into a plurality of exons by introns; therefore, it is impossible to accurately predict the structure of the protein encoded by the gene solely from the sequence information on the gene. In contrast, for a cDNA prepared from intron-excluded mRNA, information on the amino acid sequence of protein is obtained as information on a single continuous sequence, enabling easy determination of the primary structure thereof.

In particular, analyzing a full-length cDNA enables the identification of the mRNA transcription initiation point on the genome sequence based on the 5′-terminal sequence of the cDNA, and also enables analysis of the stability of mRNA contained in the sequence and of factors involved in expression control in the translation stage. Also, because the ATG codon, which serves as the translation initiation point, is present on the 5′ side, translation into protein in the right frame can be achieved. Therefore, by using an appropriate gene expression system, it is also possible to mass-produce the protein encoded by the cDNA, and to express the protein and analyze the biological activity thereof. Hence, it is considered that by performing an analysis using a protein expressed from full-length cDNA, important information that could not be obtained solely by genome sequence analysis is obtained, and that it is possible to discover novel target proteins that do not lie in the conventional category of drug discovery target proteins.

### DISCLOSURE OF THE INVENTION

The objects of the present invention are to provide target proteins and target genes for the development of bioactive substances (e.g., drug discovery), and various means that enable the development of novel bioactive substances using the same and the like.

The present inventors diligently investigated new drug innovation target proteins that can be useful for the development of new drugs, by analyzing interactions between human proteins and compounds that have been used as drugs by the SEC-MS method, and found novel target proteins and novel target genes that are useful for the development of bioactive substances, for example, drug discovery. The present inventors conducted further investigations based on this finding, conceived that substances that regulate the expression or function of these genes are capable of regulating various bioactivities, and that substances capable of regulating various bioactivities are developed by screening substances that regulate the expression or function of these genes, and by derivatizing these bioactive substances so that the expression or function of the target genes therefor can be regulated, and the like, and completed the present invention.

Accordingly, the present invention provides the followings:
[1] a method for screening a substance capable of regulating an action associated with a bioactive substance X, which comprises determining whether or not a test substance is capable of regulating the expression or function of a target protein Y or a gene that encodes the protein, wherein the combination of the bioactive substance X and the target protein Y is any of the following (a1) to (a192) (where necessary, to be abbreviated as “combination A”): (a1) a combination of trimethylcolchicic acid and a protein comprising the amino acid sequence shown by SEQ ID NO:1 or SEQ ID NO:2 or a protein homologous thereto or a variant thereof; (a2) a combination of acenocoumarol and a protein comprising the amino acid sequence shown by SEQ ID NO:27 or a protein homologous thereto or a variant thereof;
(a3) a combination of paracetamol and a protein comprising the amino acid sequence shown by SEQ ID NO:3 or a protein homologous thereto or a variant thereof;
(a4) a combination of acetohexamide and a protein comprising the amino acid sequence shown by SEQ ID NO:23, SEQ ID NO:24 or SEQ ID NO:34 or a protein homologous thereto or a variant thereof;
(a5) a combination of acetopromazine and a protein comprising the amino acid sequence shown by SEQ ID NO:36 or a protein homologous thereto or a variant thereof;
(a6) a combination of actinomycin D and a protein comprising the amino acid sequence shown by SEQ ID NO:54 or a protein homologous thereto or a variant thereof;
(a7) a combination of ajmaline and a protein comprising the amino acid sequence shown by SEQ ID NO:1 or SEQ ID NO:2 or a protein homologous thereto or a variant thereof;
(a8) a combination of albendazole and a protein comprising the amino acid sequence shown by SEQ ID NO:38 or a protein homologous thereto or a variant thereof;
(a9) a combination of alfuzosin and a protein comprising the amino acid sequence shown by SEQ ID NO:35 or a protein homologous thereto or a variant thereof;
(a10) a combination of α-methyl-5-hydroxytryptamine and a protein comprising the amino acid sequence shown by SEQ ID NO:30 or a protein homologous thereto or a variant thereof;
(a11) a combination of amoxapine and a protein comprising the amino acid sequence shown by SEQ ID NO:36 or a protein homologous thereto or a variant thereof;
(a12) a combination of antipyrine and a protein comprising the amino acid sequence shown by SEQ ID NO:1 or a protein homologous thereto or a variant thereof;
(a13) a combination of azithromycin and a protein comprising the amino acid sequence shown by SEQ ID NO:62 or a protein homologous thereto or a variant thereof;
(a14) a combination of benzbromarone and a protein comprising the amino acid sequence shown by SEQ ID NO:42, SEQ ID NO:53 or SEQ ID NO:54 or a protein homologous thereto or a variant thereof;
(a15) a combination of benzethonium and a protein comprising the amino acid sequence shown by SEQ ID NO:23, SEQ ID NO:53 or SEQ ID NO:61 or a protein homologous thereto or a variant thereof;
(a16) a combination of benzydamine and a protein comprising the amino acid sequence shown by SEQ ID NO:60 or a protein homologous thereto or a variant thereof;
(a17) a combination of berberine and a protein comprising the amino acid sequence shown by SEQ ID NO:32 or a protein homologous thereto or a variant thereof;
(a18) a combination of bezafibrate and a protein comprising the amino acid sequence shown by SEQ ID NO:39 or a protein homologous thereto or a variant thereof;
(a19) a combination of bicartamide and a protein comprising the amino acid sequence shown by SEQ ID NO:53 or a protein homologous thereto or a variant thereof;
(a20) a combination of boldine and a protein comprising the amino acid sequence shown by SEQ ID NO:1 or a protein homologous thereto or a variant thereof;
(a21) a combination of bromperidol and a protein comprising the amino acid sequence shown by SEQ ID NO:33 or a protein homologous thereto or a variant thereof;
(a22) a combination of budesonide and a protein comprising the amino acid sequence shown by SEQ ID NO:27 or a protein homologous thereto or a variant thereof;
(a23) a combination of bupivacaine and a protein comprising the amino acid sequence shown by SEQ ID NO:14 or a protein homologous thereto or a variant thereof;
(a24) a combination of buspirone and a protein comprising the amino acid sequence shown by SEQ ID NO:29 or a protein homologous thereto or a variant thereof;
(a25) a combination of cefazolin and a protein comprising the amino acid sequence shown by SEQ ID NO:59 or a protein homologous thereto or a variant thereof;
(a26) a combination of celestine blue and a protein comprising the amino acid sequence shown by SEQ ID NO:1, SEQ ID NO:2, SEQ ID NO:3, SEQ ID NO:32 or SEQ ID NO:46 or a protein homologous thereto or a variant thereof;
(a27) a combination of cephaeline and a protein comprising the amino acid sequence shown by SEQ ID NO:1 or SEQ ID NO:36 or a protein homologous thereto or a variant thereof;
(a28) a combination of chlordiazepoxide and a protein comprising the amino acid sequence shown by SEQ ID NO:56 or a protein homologous thereto or a variant thereof;
(a29) a combination of chlorogenic acid and a protein comprising the amino acid sequence shown by SEQ ID NO:27 or a protein homologous thereto or a variant thereof;
(a30) a combination of chlorothiazide and a protein comprising the amino acid sequence shown by SEQ ID NO:27 or a protein homologous thereto or a variant thereof;
(a31) a combination of chromomycin A3 and a protein comprising the amino acid sequence shown by SEQ ID NO:17 or SEQ ID NO:34 or a protein homologous thereto or a variant thereof;
(a32) a combination of ciclopirox and a protein comprising the amino acid sequence shown by SEQ ID NO:1 or SEQ ID NO:3 or a protein homologous thereto or a variant thereof;
(a33) a combination of cisapride and a protein comprising the amino acid sequence shown by SEQ ID NO:31 or a protein homologous thereto or a variant thereof;
(a34) a combination of clarithromycin and a protein comprising the amino acid sequence shown by SEQ ID NO:49 or a protein homologous thereto or a variant thereof;
(a35) a combination of clemizole and a protein comprising the amino acid sequence shown by SEQ ID NO:22 or SEQ ID NO:47 or a protein homologous thereto or a variant thereof;
(a36) a combination of clenbuterol and a protein comprising the amino acid sequence shown by SEQ ID NO:23, SEQ ID NO:36 or SEQ ID NO:60 or a protein homologous thereto or a variant thereof;
(a37) a combination of clobetasone and a protein comprising the amino acid sequence shown by SEQ ID NO:35 or a protein homologous thereto or a variant thereof;
(a38) a combination of clofazimine and a protein comprising the amino acid sequence shown by SEQ ID NO:15, SEQ ID NO:37, SEQ ID NO:53 or SEQ ID NO:54 or a protein homologous thereto or a variant thereof;
(a39) a combination of clofilium and a protein comprising the amino acid sequence shown by SEQ ID NO:1 or a protein homologous thereto or a variant thereof;
(a40) a combination of clomiphene and a protein comprising the amino acid sequence shown by SEQ ID NO:23 or a protein homologous thereto or a variant thereof;
(a41) a combination of clopamide and a protein comprising the amino acid sequence shown by SEQ ID NO:23 or a protein homologous thereto or a variant thereof;
(a42) a combination of colchicine and a protein comprising the amino acid sequence shown by SEQ ID NO:59 or a protein homologous thereto or a variant thereof;
(a43) a combination of colistin and a protein comprising the amino acid sequence shown by SEQ ID NO:62 or a protein homologous thereto or a variant thereof;
(a44) a combination of conessine and a protein comprising the amino acid sequence shown by SEQ ID NO:1 or a protein homologous thereto or a variant thereof;
(a45) a combination of coniine (DL) and a protein comprising the amino acid sequence shown by SEQ ID NO:1 or SEQ ID NO:3 or a protein homologous thereto or a variant thereof;
(a46) a combination of coralyne and a protein comprising the amino acid sequence shown by SEQ ID NO:33 or a protein homologous thereto or a variant thereof;
(a47) a combination of cyclobenzaprine and a protein comprising the amino acid sequence shown by SEQ ID NO:23 or a protein homologous thereto or a variant thereof;
(a48) a combination of cyclopentolate and a protein comprising the amino acid sequence shown by SEQ ID NO:36 or a protein homologous thereto or a variant thereof;
(a49) a combination of cyclosporine A and a protein comprising the amino acid sequence shown by SEQ ID NO:50 or a protein homologous thereto or a variant thereof;
(a50) a combination of diclofenac and a protein comprising the amino acid sequence shown by SEQ ID NO:27 or a protein homologous thereto or a variant thereof;
(a51) a combination of dichlorphenamide and a protein comprising the amino acid sequence shown by SEQ ID NO:51 or a protein homologous thereto or a variant thereof;
(a52) a combination of diflunisal and a protein comprising the amino acid sequence shown by SEQ ID NO:32 or a protein homologous thereto or a variant thereof;
(a53) a combination of dihydrostreptomycin and a protein comprising the amino acid sequence shown by SEQ ID NO:19 or a protein homologous thereto or a variant thereof;
(a54) a combination of diperodon and a protein comprising the amino acid sequence shown by SEQ ID NO:27 or a protein homologous thereto or a variant thereof;
(a55) a combination of difenidol and a protein comprising the amino acid sequence shown by SEQ ID NO:1 or a protein homologous thereto or a variant thereof;
(a56) a combination of dipyridamole and a protein comprising the amino acid sequence shown by SEQ ID NO:15 or a protein homologous thereto or a variant thereof;
(a57) a combination of dizocilpine and a protein comprising the amino acid sequence shown by SEQ ID NO:25 or a protein homologous thereto or a variant thereof;
(a58) a combination of DO897/99 and a protein comprising the amino acid sequence shown by SEQ ID NO:27 or SEQ ID NO:34 or a protein homologous thereto or a variant thereof;
(a59) a combination of domperidone and a protein comprising the amino acid sequence shown by SEQ ID NO:34, SEQ ID NO:36, SEQ ID NO:53 or SEQ ID NO:54 or a protein homologous thereto or a variant thereof;
(a60) a combination of dopamine and a protein comprising the amino acid sequence shown by SEQ ID NO:30 or a protein homologous thereto or a variant thereof;
(a61) a combination of doxazosin and a protein comprising the amino acid sequence shown by SEQ ID NO:1, SEQ ID NO:35, SEQ ID NO:53 or SEQ ID NO:61 or a protein homologous thereto or a variant thereof;
(a62) a combination of doxycycline and a protein comprising the amino acid sequence shown by SEQ ID NO:59 or a protein homologous thereto or a variant thereof;
(a63) a combination of eburnamonine and a protein comprising the amino acid sequence shown by SEQ ID NO:10 or SEQ ID NO:44 or a protein homologous thereto or a variant thereof;
(a64) a combination of etodolac and a protein comprising the amino acid sequence shown by SEQ ID NO:23 or a protein homologous thereto or a variant thereof;
(a65) a combination of fenbendazole and a protein comprising the amino acid sequence shown by SEQ ID NO:22 or a protein homologous thereto or a variant thereof;
(a66) a combination of fenbufen and a protein comprising the amino acid sequence shown by SEQ ID NO:59 or a protein homologous thereto or a variant thereof;
(a67) a combination of fenoprofen and a protein comprising the amino acid sequence shown by SEQ ID NO:26 or a protein homologous thereto or a variant thereof;
(a68) a combination of flumequine and a protein comprising the amino acid sequence shown by SEQ ID NO:56 or a protein homologous thereto or a variant thereof;
(a69) a combination of flupentixol and a protein comprising the amino acid sequence shown by SEQ ID NO:23 or SEQ ID NO:34 or a protein homologous thereto or a variant thereof;
(a70) a combination of fluphenazine and a protein comprising the amino acid sequence shown by SEQ ID NO:34 or SEQ ID NO:61 or a protein homologous thereto or a variant thereof;
(a71) a combination of fluvoxamine and a protein comprising the amino acid sequence shown by SEQ ID NO:25 or a protein homologous thereto or a variant thereof;
(a72) a combination of furazolidone and a protein comprising the amino acid sequence shown by SEQ ID NO:52 or a protein homologous thereto or a variant thereof;
(a73) a combination of gabapentin and a protein comprising the amino acid sequence shown by SEQ ID NO:59 or a protein homologous thereto or a variant thereof;
(a74) a combination of GBR12909 and a protein comprising the amino acid sequence shown by SEQ ID NO:61 or a protein homologous thereto or a variant thereof;
(a75) a combination of glibenclamide and a protein comprising the amino acid sequence shown by SEQ ID NO:23 or SEQ ID NO:37 or a protein homologous thereto or a variant thereof;
(a76) a combination of glipizide and a protein comprising the amino acid sequence shown by SEQ ID NO:23 or a protein homologous thereto or a variant thereof;
(a77) a combination of gramicidin and a protein comprising the amino acid sequence shown by SEQ ID NO:53 or a protein homologous thereto or a variant thereof;
(a78) a combination of guanfacine and a protein comprising the amino acid sequence shown by SEQ ID NO:23 or a protein homologous thereto or a variant thereof;
(a79) a combination of harmol and a protein comprising the amino acid sequence shown by SEQ ID NO:22 or a protein homologous thereto or a variant thereof;
(a80) a combination of hydroflumethiazide and a protein comprising the amino acid sequence shown by SEQ ID NO:11 or a protein homologous thereto or a variant thereof;
(a81) a combination of hydroxychloroquine and a protein comprising the amino acid sequence shown by SEQ ID NO:52 or a protein homologous thereto or a variant thereof;
(a82) a combination of hydroxytacrine(R,S) and a protein comprising the amino acid sequence shown by SEQ ID NO:43 or a protein homologous thereto or a variant thereof;
(a83) a combination of ifosfamide and a protein comprising the amino acid sequence shown by SEQ ID NO:22 or a protein homologous thereto or a variant thereof;
(a84) a combination of iobenguane and a protein comprising the amino acid sequence shown by SEQ ID NO:9 or a protein homologous thereto or a variant thereof;
(a85) a combination of iproniazid and a protein comprising the amino acid sequence shown by SEQ ID NO:19 or a protein homologous thereto or a variant thereof;
(a86) a combination of isoxicam and a protein comprising the amino acid sequence shown by SEQ ID NO:23 or a protein homologous thereto or a variant thereof;
(a87) a combination of isradipine and a protein comprising the amino acid sequence shown by SEQ ID NO:24 or a protein homologous thereto or a variant thereof;
(a88) a combination of josamycin and a protein comprising the amino acid sequence shown by SEQ ID NO:49 or a protein homologous thereto or a variant thereof;
(a89) a combination of ketoprofen and a protein comprising the amino acid sequence shown by SEQ ID NO:59 or a protein homologous thereto or a variant thereof;
(a90) a combination of 3-hydroxykynurenine and a protein comprising the amino acid sequence shown by SEQ ID NO:25 or a protein homologous thereto or a variant thereof;
(a91) a combination of leuprolide and a protein comprising the amino acid sequence shown by SEQ ID NO:50 or a protein homologous thereto or a variant thereof;
(a92) a combination of L-thyroxine and a protein comprising the amino acid sequence shown by SEQ ID NO:34 or a protein homologous thereto or a variant thereof;
(a93) a combination of lidoflazine and a protein comprising the amino acid sequence shown by SEQ ID NO:59 or a protein homologous thereto or a variant thereof;
(a94) a combination of α-lobeline (−) and a protein comprising the amino acid sequence shown by SEQ ID NO:6 or a protein homologous thereto or a variant thereof;
(a95) a combination of loperamide and a protein comprising the amino acid sequence shown by SEQ ID NO:15 or SEQ ID NO:54 or a protein homologous thereto or a variant thereof;
(a96) a combination of maprotiline and a protein comprising the amino acid sequence shown by SEQ ID NO:23 or SEQ ID NO:63 or a protein homologous thereto or a variant thereof;
(a97) a combination of mebendazole and a protein comprising the amino acid sequence shown by SEQ ID NO:32 or a protein homologous thereto or a variant thereof;
(a98) a combination of meclofenamic acid and a protein comprising the amino acid sequence shown by SEQ ID NO:17 or a protein homologous thereto or a variant thereof;
(a99) a combination of metanephrine (D,L) a protein comprising the amino acid sequence shown by SEQ ID NO:52 or a protein homologous thereto or a variant thereof;
(a100) a combination of metaproterenol and a protein comprising the amino acid sequence shown by SEQ ID NO:43 or a protein homologous thereto or a variant thereof;
(a101) a combination of metergotamine and a protein comprising the amino acid sequence shown by SEQ ID NO:25 or SEQ ID NO:43 or a protein homologous thereto or a variant thereof;
(a102) a combination of methimazole and a protein comprising the amino acid sequence shown by SEQ ID NO:12 or a protein homologous thereto or a variant thereof;
(a103) a combination of methoxamine and a protein comprising the amino acid sequence shown by SEQ ID NO:25 or a protein homologous thereto or a variant thereof;
(a104) a combination of methoxy-6-harmalan and a protein comprising the amino acid sequence shown by SEQ ID NO:1 or SEQ ID NO:2 or a protein homologous thereto or a variant thereof;
(a105) a combination of mifepristone and a protein comprising the amino acid sequence shown by SEQ ID NO:42 or a protein homologous thereto or a variant thereof;
(a106) a combination of minaprine and a protein comprising the amino acid sequence shown by SEQ ID NO:2 or a protein homologous thereto or a variant thereof;
(a107) a combination of minocycline and a protein comprising the amino acid sequence shown by SEQ ID NO:36 or a protein homologous thereto or a variant thereof;
(a108) a combination of misoprostol and a protein comprising the amino acid sequence shown by SEQ ID NO:23 or a protein homologous thereto or a variant thereof;
(a109) a combination of molsidomine and a protein comprising the amino acid sequence shown by SEQ ID NO:4 or a protein homologous thereto or a variant thereof;
(a110) a combination of moroxydine and a protein comprising the amino acid sequence shown by SEQ ID NO:7 or a protein homologous thereto or a variant thereof;
(a111) a combination of moxalactam and a protein comprising the amino acid sequence shown by SEQ ID NO:36 or a protein homologous thereto or a variant thereof;
(a112) a combination of mupirocin and a protein comprising the amino acid sequence shown by SEQ ID NO:24 or a protein homologous thereto or a variant thereof;
(a113) a combination of nefopam and a protein comprising the amino acid sequence shown by SEQ ID NO:19 or a protein homologous thereto or a variant thereof;
(a114) a combination of nicardipine and a protein comprising the amino acid sequence shown by SEQ ID NO:54 or a protein homologous thereto or a variant thereof;
(a115) a combination of nimesulide and a protein comprising the amino acid sequence shown by SEQ ID NO:27 or a protein homologous thereto or a variant thereof;
(a116) a combination of norharman and a protein comprising the amino acid sequence shown by SEQ ID NO:45 or a protein homologous thereto or a variant thereof;
(a117) a combination of oxytocin and a protein comprising the amino acid sequence shown by SEQ ID NO:49 or a protein homologous thereto or a variant thereof;
(a118) a combination of paroxetine and a protein comprising the amino acid sequence shown by SEQ ID NO:1 or SEQ ID NO:25 or a protein homologous thereto or a variant thereof;
(a119) a combination of perhexiline and a protein comprising the amino acid sequence shown by SEQ ID NO:23 or SEQ ID NO:36 or a protein homologous thereto or a variant thereof;
(a120) a combination of phenformin and a protein comprising the amino acid sequence shown by SEQ ID NO:36 or a protein homologous thereto or a variant thereof;
(a121) a combination of pimethixene and a protein comprising the amino acid sequence shown by SEQ ID NO:1 or a protein homologous thereto or a variant thereof;
(a122) a combination of piperlongumine and a protein comprising the amino acid sequence shown by SEQ ID NO:22 or a protein homologous thereto or a variant thereof;
(a123) a combination of pirenzepine and a protein comprising the amino acid sequence shown by SEQ ID NO:40 or a protein homologous thereto or a variant thereof;
(a124) a combination of probenecid and a protein comprising the amino acid sequence shown by SEQ ID NO:23 or SEQ ID NO:59 or a protein homologous thereto or a variant thereof;
(a125) a combination of procaine and a protein comprising the amino acid sequence shown by SEQ ID NO:61 or a protein homologous thereto or a variant thereof;
(a126) a combination of propranolol and a protein comprising the amino acid sequence shown by SEQ ID NO:22 or a protein homologous thereto or a variant thereof;
(a127) a combination of protriptyline and a protein comprising the amino acid sequence shown by SEQ ID NO:63 or a protein homologous thereto or a variant thereof;
(a128) a combination of pyrilamine and a protein comprising the amino acid sequence shown by SEQ ID NO:36 or SEQ ID NO:45 or a protein homologous thereto or a variant thereof;
(a129) a combination of quercetin and a protein comprising the amino acid sequence shown by SEQ ID NO:20 or SEQ ID NO:54 or a protein homologous thereto or a variant thereof;
(a130) a combination of quinacrine and a protein comprising the amino acid sequence shown by SEQ ID NO:61 or a protein homologous thereto or a variant thereof;
(a131) a combination of quinine and a protein comprising the amino acid sequence shown by SEQ ID NO:1 or SEQ ID NO:10 or a protein homologous thereto or a variant thereof;
(a132) a combination of rescinnamine and a protein comprising the amino acid sequence shown by SEQ ID NO:41 or SEQ ID NO:53 or a protein homologous thereto or a variant thereof;
(a133) a combination of risperidone and a protein comprising the amino acid sequence shown by SEQ ID NO:13 or SEQ ID NO:35 or a protein homologous thereto or a variant thereof;
(a134) a combination of ritodrine and a protein comprising the amino acid sequence shown by SEQ ID NO:1 or SEQ ID NO:2 or a protein homologous thereto or a variant thereof;
(a135) a combination of saquinavir and a protein comprising the amino acid sequence shown by SEQ ID NO:17 or SEQ ID NO:53 or a protein homologous thereto or a variant thereof;
(a136) a combination of scoulerine and a protein comprising the amino acid sequence shown by SEQ ID NO:2 or a protein homologous thereto or a variant thereof;
(a137) a combination of sulfadimethoxine and a protein comprising the amino acid sequence shown by SEQ ID NO:1 or a protein homologous thereto or a variant thereof;
(a138) a combination of sulfaphenazole and a protein comprising the amino acid sequence shown by SEQ ID NO:23 or a protein homologous thereto or a variant thereof;
(a139) a combination of syrosingopine and a protein comprising the amino acid sequence shown by SEQ ID NO:53 or a protein homologous thereto or a variant thereof;
(a140) a combination of tamoxifen and a protein comprising the amino acid sequence shown by SEQ ID NO:3 or a protein homologous thereto or a variant thereof;
(a141) a combination of terconazole and a protein comprising the amino acid sequence shown by SEQ ID NO:36 or a protein homologous thereto or a variant thereof;
(a142) a combination of thioproperazine and a protein comprising the amino acid sequence shown by SEQ ID NO:23 or SEQ ID NO:27 or a protein homologous thereto or a variant thereof;
(a143) a combination of thiothixene(cis) and a protein comprising the amino acid sequence shown by SEQ ID NO:23 or a protein homologous thereto or a variant thereof;
(a144) a combination of tobramycin and a protein comprising the amino acid sequence shown by SEQ ID NO:36 or a protein homologous thereto or a variant thereof;
(a145) a combination of tolbutamide and a protein comprising the amino acid sequence shown by SEQ ID NO:23 or a protein homologous thereto or a variant thereof;
(a146) a combination of trifluoperazine and a protein comprising the amino acid sequence shown by SEQ ID NO:34 or a protein homologous thereto or a variant thereof;
(a147) a combination of trimetazidine and a protein comprising the amino acid sequence shown by SEQ ID NO:5 or a protein homologous thereto or a variant thereof;
(a148) a combination of viloxazine and a protein comprising the amino acid sequence shown by SEQ ID NO:58 or a protein homologous thereto or a variant thereof;
(a149) a combination of xylazine and a protein comprising the amino acid sequence shown by SEQ ID NO:8 or a protein homologous thereto or a variant thereof;
(a150) a combination of acetylsalicylsalicylic acid and a protein comprising the amino acid sequence shown by SEQ ID NO:28 or a protein homologous thereto or a variant thereof;
(a151) a combination of nimetazepam and a protein comprising the amino acid sequence shown by SEQ ID NO:25 or a protein homologous thereto or a variant thereof;
(a152) a combination of clobazam and a protein comprising the amino acid sequence shown by SEQ ID NO:48 or a protein homologous thereto or a variant thereof;
(a153) a combination of alimemazine and a protein comprising the amino acid sequence shown by SEQ ID NO:1 or SEQ ID NO:2 or a protein homologous thereto or a variant thereof;
(a154) a combination of tranilast and a protein comprising the amino acid sequence shown by SEQ ID NO:32 or a protein homologous thereto or a variant thereof;
(a155) a combination of ebastine and a protein comprising the amino acid sequence shown by SEQ ID NO:54 or a protein homologous thereto or a variant thereof;
(a156) a combination of pranlukast and a protein comprising the amino acid sequence shown by SEQ ID NO:18, SEQ ID NO:20, SEQ ID NO:21, SEQ ID NO:35, SEQ ID NO:42, SEQ ID NO:53 or SEQ ID NO:54 or a protein homologous thereto or a variant thereof;
(a157) a combination of methyclothiazide and a protein comprising the amino acid sequence shown by SEQ ID NO:16 or SEQ ID NO:23 or a protein homologous thereto or a variant thereof;
(a158) a combination of alacepril and a protein comprising the amino acid sequence shown by SEQ ID NO:24 or a protein homologous thereto or a variant thereof;
(a159) a combination of clinofibrate and a protein comprising the amino acid sequence shown by SEQ ID NO:23 or SEQ ID NO:34 or a protein homologous thereto or a variant thereof;
(a160) a combination of acetylcysteine and a protein comprising the amino acid sequence shown by SEQ ID NO:2 or SEQ ID NO:3 or a protein homologous thereto or a variant thereof;
(a161) a combination of buformin and a protein comprising the amino acid sequence shown by SEQ ID NO:57 or a protein homologous thereto or a variant thereof;
(a162) a combination of terguride and a protein comprising the amino acid sequence shown by SEQ ID NO:9 or a protein homologous thereto or a variant thereof;
(a163) a combination of stanozolol and a protein comprising the amino acid sequence shown by SEQ ID NO:16 or a protein homologous thereto or a variant thereof;
(a164) a combination of mestanolone and a protein comprising the amino acid sequence shown by SEQ ID NO:42 or a protein homologous thereto or a variant thereof;
(a165) a combination of pantethine and a protein comprising the amino acid sequence shown by SEQ ID NO:1 or a protein homologous thereto or a variant thereof;
(a166) a combination of limaprost and a protein comprising the amino acid sequence shown by SEQ ID NO:24 or a protein homologous thereto or a variant thereof;
(a167) a combination of sarpogrelate and a protein comprising the amino acid sequence shown by SEQ ID NO:27 or a protein homologous thereto or a variant thereof;
(a168) a combination of argatroban and a protein comprising the amino acid sequence shown by SEQ ID NO:23 or a protein homologous thereto or a variant thereof;
(a169) a combination of fludroxycortide and a protein comprising the amino acid sequence shown by SEQ ID NO:25 or a protein homologous thereto or a variant thereof;
(a170) a combination of sulfadoxine and a protein comprising the amino acid sequence shown by SEQ ID NO:23 or a protein homologous thereto or a variant thereof;
(a171) a combination of ubenimex and a protein comprising the amino acid sequence shown by SEQ ID NO:23 or a protein homologous thereto or a variant thereof;
(a172) a combination of celecoxib and a protein comprising the amino acid sequence shown by SEQ ID NO:23 or a protein homologous thereto or a variant thereof;
(a173) a combination of 6-furfurylaminopurine and a protein comprising the amino acid sequence shown by SEQ ID NO:57 or a protein homologous thereto or a variant thereof;
(a174) a combination of solasodine and a protein comprising the amino acid sequence shown by SEQ ID NO:24 or a protein homologous thereto or a variant thereof;
(a175) a combination of gossypol and a protein comprising the amino acid sequence shown by SEQ ID NO:23 or a protein homologous thereto or a variant thereof;
(a176) a combination of fluorocurarine and a protein comprising the amino acid sequence shown by SEQ ID NO:10 or a protein homologous thereto or a variant thereof;
(a177) a combination of pempidine and a protein comprising the amino acid sequence shown by. SEQ ID NO:57 or a protein homologous thereto or a variant thereof;
(a178) a combination of nitrarine and a protein comprising the amino acid sequence shown by SEQ ID NO:46 or SEQ ID NO:57 or a protein homologous thereto or a variant thereof;
(a179) a combination of promazine and a protein comprising the amino acid sequence shown by SEQ ID NO:18 or a protein homologous thereto or a variant thereof;
(a180) a combination of sulfabenzamide and a protein comprising the amino acid sequence shown by SEQ ID NO:23 or a protein homologous thereto or a variant thereof;
(a181) a combination of althiazide and a protein comprising the amino acid sequence shown by SEQ ID NO:23 or a protein homologous thereto or a variant thereof;
(a182) a combination of α-ergocryptine and a protein comprising the amino acid sequence shown by SEQ ID NO:23 or SEQ ID NO:53 or a protein homologous thereto or a variant thereof;
(a183) a combination of ebselen and a protein comprising the amino acid sequence shown by SEQ ID NO:6 or a protein homologous thereto or a variant thereof;
(a184) a combination of furaltadone and a protein comprising the amino acid sequence shown by SEQ ID NO:10 or a protein homologous thereto or a variant thereof;
(a185) a combination of pyrithyldione and a protein comprising the amino acid sequence shown by SEQ ID NO:55 or a protein homologous thereto or a variant thereof;
(a186) a combination of benzthiazide and a protein comprising the amino acid sequence shown by SEQ ID NO:23 or SEQ ID NO:51 or a protein homologous thereto or a variant thereof;
(a187) a combination of levobunolol and a protein comprising the amino acid sequence shown by SEQ ID NO:44 or a protein homologous thereto or a variant thereof;
(a188) a combination of raloxifene and a protein comprising the amino acid sequence shown by SEQ ID NO:37 or a protein homologous thereto or a variant thereof;
(a189) a combination of luteolin and a protein comprising the amino acid sequence shown by SEQ ID NO:20 or SEQ ID NO:54 or a protein homologous thereto or a variant thereof;
(a190) a combination of valdecoxib and a protein comprising the amino acid sequence shown by SEQ ID NO:23 or a protein homologous thereto or a variant thereof;
(a191) a combination of carboprost and a protein comprising the amino acid sequence shown by SEQ ID NO:24 or SEQ ID NO:34 or a protein homologous thereto or a variant thereof;
(a192) a combination of gabexate and a protein comprising the amino acid sequence shown by SEQ ID NO:23 or a protein homologous thereto or a variant thereof.
[2] The method according to [1] above, which comprises the following steps (a) to (c):
(a) a step for bringing the test substance into contact with the target protein Y;
(b) a step for measuring the functional level of the protein in the presence of the test substance, and comparing said functional level with the functional level of the protein in the absence of the test substance;
(c) a step for selecting a test substance that alters the functional level of the protein on the basis of the result of the comparison in (b) above.
[3] The method according to [1] above, which comprises the following steps (a) to (c):
(a) a step for bringing the test substance into contact with cells allowing a measurement of the expression of the target protein Y or a gene that encodes the protein;
(b) a step for measuring the expression level of the gene in cells in contact with the test substance, and comparing said expression level with the expression level of the gene in control cells not in contact with the test substance;
(c) a step for selecting a test substance that regulates the expression level of the gene on the basis of the result of the comparison in (b) above.
[4] The method according to [1] above, which comprises the following steps (a) to (c):
(a) a step for bringing the test substance into contact with the target protein Y;
(b) a step for measuring the ability of the test substance to bind to the protein;
(c) a step for selecting a test substance capable of binding to the protein on the basis of the result from (b) above.
[5] The method according to [1] above, which comprises the following steps (a) to (c):
(a) a step for bringing the test substance and a target protein Y-binding substance into contact with the target protein Y;
(b) a step for measuring the ability of the target protein Y-binding substance to bind to the protein in the presence of the test substance, and comparing said ability with the ability of the target protein Y-binding substance to bind to the protein in the absence of the test substance;
(c) a step for selecting a test substance that alters the ability of the target protein Y-binding substance to bind to the protein on the basis of the result of the comparison in (b) above.
[6] A method for screening a substance capable of regulating a function associated with a target protein Y, which comprises comparing the ability of a test substance to bind to the target protein Y or the action associated with the test compound, with the ability of a bioactive substance X to bind to the target protein Y or the action associated with the bioactive substance, wherein the combination of the target protein Y and the bioactive substance X is any of the following (b1) to (b63) (where necessary, to be abbreviated as “combination B”):
(b1) a combination of a protein comprising the amino acid sequence shown by SEQ ID NO:1 or a protein homologous thereto or a variant thereof and ajmaline, celestine blue, conessine, difenidol, methoxy-6-harmalan, pimethixene, quinine, ritodrine, alimemazine, boldine, clofilium, paroxetine, trimethylcolchicic acid, antipyrine, cephaeline, ciclopirox, coniine (DL), doxazosin, sulfadimethoxine, pantethine or a derivative thereof capable of binding to the protein;
(b2) a combination of a protein comprising the amino acid sequence shown by SEQ ID NO:2 or a protein homologous thereto or a variant thereof and trimethylcolchicic acid, ajmaline, celestine blue, methoxy-6-harmalan, minaprine, ritodrine, scoulerine, alimemazine, acetylcysteine or a derivative thereof capable of binding to the protein;
(b3) a combination of a protein comprising the amino acid sequence shown by SEQ ID NO:3 or a protein homologous thereto or a variant thereof and celestine blue, ciclopirox, coniine (DL), tamoxifen, acetylcysteine, paracetamol or a derivative thereof capable of binding to the protein;
(b4) a combination of a protein comprising the amino acid sequence shown by SEQ ID NO:4 or a protein homologous thereto or a variant thereof and molsidomine or a derivative thereof capable of binding to the protein;
(b5) a combination of a protein comprising the amino acid sequence shown by SEQ ID NO:5 or a protein homologous thereto or a variant thereof and trimetazidine or a derivative thereof capable of binding to the protein;
(b6) a combination of a protein comprising the amino acid sequence shown by SEQ ID NO:6 or a protein homologous thereto or a variant thereof and α-lobeline (−), ebselen or a derivative thereof capable of binding to the protein;
(b7) a combination of a protein comprising the amino acid sequence shown by SEQ ID NO:7 or a protein homologous thereto or a variant thereof and moroxydine or a derivative thereof capable of binding to the protein;
(b8) a combination of a protein comprising the amino acid sequence shown by SEQ ID NO:8 or a protein homologous thereto or a variant thereof and xylazine or a derivative thereof capable of binding to the protein;
(b9) a combination of a protein comprising the amino acid sequence shown by SEQ ID NO:9 or a protein homologous thereto or a variant thereof and terguride, iobenguane or a derivative thereof capable of binding to the protein;
(b10) a combination of a protein comprising the amino acid sequence shown by SEQ ID NO:10 or a protein homologous thereto or a variant thereof and quinine, eburnamonine, fluorocurarine, furaltadone or a derivative thereof capable of binding to the protein;
(b11) a combination of a protein comprising the amino acid sequence shown by SEQ ID NO:11 or a protein homologous thereto or a variant thereof and hydroflumethiazide or a derivative thereof capable of binding to the protein;
(b12) a combination of a protein comprising the amino acid sequence shown by SEQ ID NO:12 or a protein homologous thereto or a variant thereof and methimazole or a derivative thereof capable of binding to the protein;
(b13) a combination of a protein comprising the amino acid sequence shown by SEQ ID NO:13 or a protein homologous thereto or a variant thereof and risperidone or a derivative thereof capable of binding to the protein;
(b14) a combination of a protein comprising the amino acid sequence shown by SEQ ID NO:14 or a protein homologous thereto or a variant thereof and bupivacaine or a derivative thereof capable of binding to the protein;
(b15) a combination of a protein comprising the amino acid sequence shown by SEQ ID NO:15 or a protein homologous thereto or a variant thereof and loperamide, clofazimine, dipyridamole or a derivative thereof capable of binding to the protein;
(b16) a combination of a protein comprising the amino acid sequence shown by SEQ ID NO:16 or a protein homologous thereto or a variant thereof and stanozolol, methyclothiazide or a derivative thereof capable of binding to the protein;
(b17) a combination of a protein comprising the amino acid sequence shown by SEQ ID NO:17 or a protein homologous thereto or a variant thereof and chromomycin A3, meclofenamic acid, saquinavir or a derivative thereof capable of binding to the is protein;
(b18) a combination of a protein comprising the amino acid sequence shown by SEQ ID NO:18 or a protein homologous thereto or a variant thereof and promazine, pranlukast or a derivative thereof capable of binding to the protein;
(b19) a combination of a protein comprising the amino acid sequence shown by SEQ ID NO:19 or a protein homologous thereto or a variant thereof and dihydrostreptomycin, iproniazid, nefopam or a derivative thereof capable of binding to the protein;
(b20) a combination of a protein comprising the amino acid sequence shown by SEQ ID NO:20 or a protein homologous thereto or a variant thereof and quercetin, luteolin, pranlukast or a derivative thereof capable of binding to the protein;
(b21) a combination of a protein comprising the amino acid sequence shown by SEQ ID NO:21 or a protein homologous thereto or a variant thereof and pranlukast or a derivative thereof capable of binding to the protein;
(b22) a combination of a protein comprising the amino acid sequence shown by SEQ ID NO:22 or a protein homologous thereto or a variant thereof and clemizole, fenbendazole, harmol, ifosfamide, piperlongumine, propranolol or a derivative thereof capable of binding to the protein;
(b23) a combination of a protein comprising the amino acid sequence shown by SEQ ID NO:23 or a protein homologous thereto or a variant thereof and acetohexamide, benzethonium, clomiphene, cyclobenzaprine, flupentixol, guanfacine, maprotiline, perhexiline, probenecid, clinofibrate, celecoxib, gossypol, althiazide, α-ergocryptine, gabexate, clenbuterol, etodolac, misoprostol, ubenimex, clopamide, glibenclamide, glipizide, isoxicam, sulfaphenazole, thioproperazine, thiothixene(cis), tolbutamide, methyclothiazide, argatroban, sulfadoxine, sulfabenzamide, benzthiazide, valdecoxib or a derivative thereof capable of binding to the protein;
(b24) a combination of a protein comprising the amino acid sequence shown by SEQ ID NO:24 or a protein homologous thereto or a variant thereof and acetohexamide, isradipine, mupirocin, limaprost, solasodine, alacepril, carboprost or a derivative thereof capable of binding to the protein;
(b25) a combination of a protein comprising the amino acid sequence shown by SEQ ID NO:25 or a protein homologous thereto or a variant thereof and metergotamine, methoxamine, paroxetine, dizocilpine, fluvoxamine, 3-hydroxykynurenine, nimetazepam, fludroxycortide or a derivative thereof capable of binding to the protein;
(b26) a combination of a protein comprising the amino acid sequence shown by SEQ ID NO:26 or a protein homologous thereto or a variant thereof and fenoprofen or a derivative thereof capable of binding to the protein;
(b27) a combination of a protein comprising the amino acid sequence shown by SEQ ID NO:27 or a protein homologous thereto or a variant thereof and acenocoumarol, budesonide, chlorogenic acid, chlorothiazide, diclofenac, diperodon, DO897/99, nimesulide, thioproperazine, sarpogrelate or a derivative thereof capable of binding to the protein;
(b28) a combination of a protein comprising the amino acid sequence shown by SEQ ID NO:28 or a protein homologous thereto or a variant thereof and acetylsalicylsalicylic acid or a derivative thereof capable of binding to the protein;
(b29) a combination of a protein comprising the amino acid sequence shown by SEQ ID NO:29 or a protein homologous thereto or a variant thereof and buspirone or a derivative thereof capable of binding to the protein;
(b30) a combination of a protein comprising the amino acid sequence shown by SEQ ID NO:30 or a protein homologous thereto or a variant thereof and dopamine, α-methyl-5-hydroxytryptamine or a derivative thereof capable of binding to the protein;
(b31) a combination of a protein comprising the amino acid sequence shown by SEQ ID NO:31 or a protein homologous thereto or a variant thereof and cisapride or a derivative thereof capable of binding to the protein;
(b32) a combination of a protein comprising the amino acid sequence shown by SEQ ID NO:32 or a protein homologous thereto or a variant thereof and berberine, celestine blue, diflunisal, mebendazole, tranilast or a derivative thereof capable of binding to the protein;
(b33) a combination of a protein comprising the amino acid sequence shown by SEQ ID NO:33 or a protein homologous thereto or a variant thereof and bromperidol, coralyne or a derivative thereof capable of binding to the protein;
(b34) a combination of a protein comprising the amino acid sequence shown by SEQ ID NO:34 or a protein homologous thereto or a variant thereof and DO897/99, domperidone, flupentixol, fluphenazine, L-thyroxine, trifluoperazine, clinofibrate, acetohexamide, chromomycin A3, carboprost or a derivative thereof capable of binding to the protein;
(b35) a combination of a protein comprising the amino acid sequence shown by SEQ ID NO:35 or a protein homologous thereto or a variant thereof and alfuzosin, clobetasone, doxazosin, pranlukast, risperidone or a derivative thereof capable of binding to the protein;
(b36) a combination of a protein comprising the amino acid sequence shown by SEQ ID NO:36 or a protein homologous thereto or a variant thereof and acetopromazine, cyclopentolate, perhexiline, phenformin, pyrilamine, terconazole, tobramycin, amoxapine, cephaeline, clenbuterol, domperidone, minocycline, moxalactam or a derivative thereof capable of binding to the protein;
(b37) a combination of a protein comprising the amino acid sequence shown by SEQ ID NO:37 or a protein homologous thereto or a variant thereof and glibenclamide, raloxifene, clofazimine or a derivative thereof capable of binding to the protein;
(b38) a combination of a protein comprising the amino acid sequence shown by SEQ ID NO:38 or a protein homologous thereto or a variant thereof and albendazole or a derivative thereof capable of binding to the protein;
(b39) a combination of a protein comprising the amino acid sequence shown by SEQ ID NO:39 or a protein homologous thereto or a variant thereof and bezafibrate or a derivative thereof capable of binding to the protein;
(b40) a combination of a protein comprising the amino acid sequence shown by SEQ ID NO:40 or a protein homologous thereto or a variant thereof and pirenzepine or a derivative thereof capable of binding to the protein;
(b41) a combination of a protein comprising the amino acid sequence shown by SEQ ID NO:41 or a protein homologous thereto or a variant thereof and rescinnamine or a derivative thereof capable of binding to the protein;
(b42) a combination of a protein comprising the amino acid sequence shown by SEQ ID NO:42 or a protein homologous thereto or a variant thereof and benzbromarone, pranlukast, mifepristone, mestanolone or a derivative thereof capable of binding to the protein;
(b43) a combination of a protein comprising the amino acid sequence shown by SEQ ID NO:43 or a protein homologous thereto or a variant thereof and hydroxytacrine(R,S), metergotamine, metaproterenol or a derivative thereof capable of binding to the protein;
(b44) a combination of a protein comprising the amino acid sequence shown by SEQ ID NO:44 or a protein homologous thereto or a variant thereof and eburnamonine, levobunolol or a derivative thereof capable of binding to the protein;
(b45) a combination of a protein comprising the amino acid sequence shown by SEQ ID NO:45 or a protein homologous thereto or a variant thereof and norharman, pyrilamine or a derivative thereof capable of binding to the protein;
(b46) a combination of a protein comprising the amino acid sequence shown by SEQ ID NO:46 or a protein homologous thereto or a variant thereof and celestine blue, nitrarine or a derivative thereof capable of binding to the protein;
(b47) a combination of a protein comprising the amino acid sequence shown by SEQ ID NO:47 or a protein homologous thereto or a variant thereof and clemizole or a derivative thereof capable of binding to the protein;
(b48) a combination of a protein comprising the amino acid sequence shown by SEQ ID NO:48 or a protein homologous thereto or a variant thereof and clobazam or a derivative thereof capable of binding to the protein;
(b49) a combination of a protein comprising the amino acid sequence shown by SEQ ID NO:49 or a protein homologous thereto or a variant thereof and josamycin, oxytocin, clarithromycin or a derivative thereof capable of binding to the protein;
(b50) a combination of a protein comprising the amino acid sequence shown by SEQ ID NO:50 or a protein homologous thereto or a variant thereof and leuprolide, cyclosporine A or a derivative thereof capable of binding to the protein;
(b51) a combination of a protein comprising the amino acid sequence shown by SEQ ID NO:51 or a protein homologous thereto or a variant thereof and dichlorphenamide, benzthiazide or a derivative thereof capable of binding to the protein;
(b52) a combination of a protein comprising the amino acid sequence shown by SEQ ID NO:52 or a protein homologous thereto or a variant thereof and hydroxychloroquine, furazolidone, metanephrine (D,L) or a derivative thereof capable of binding to the protein;
(b53) a combination of a protein comprising the amino acid sequence shown by SEQ ID NO:53 or a protein homologous thereto or a variant thereof and benzbromarone, benzethonium, clofazimine, domperidone, doxazosin, gramicidin, α-ergocryptine, bicartamide, rescinnamine, saquinavir, syrosingopine, pranlukast or a derivative thereof capable of binding to the protein;
(b54) a combination of a protein comprising the amino acid sequence shown by SEQ ID NO:54 or a protein homologous thereto or a variant thereof and benzbromarone, clofazimine, domperidone, nicardipine, quercetin, ebastine, actinomycin D, loperamide, pranlukast, luteolin or a derivative thereof capable of binding to the protein;
(b55) a combination of a protein comprising the amino acid sequence shown by SEQ ID NO:55 or a protein homologous thereto or a variant thereof and pyrithyldione or a derivative thereof capable of binding to the protein;
(b56) a combination of a protein comprising the amino acid sequence shown by SEQ ID NO:56 or a protein homologous thereto or a variant thereof and chlordiazepoxide, flumequine or a derivative thereof capable of binding to the protein;
(b57) a combination of a protein comprising the amino acid sequence shown by SEQ ID NO:57 or a protein homologous thereto or a variant thereof and buformin, 6-furfurylaminopurine, nitrarine, pempidine or a derivative thereof capable of binding to the protein;
(b58) a combination of a protein comprising the amino acid sequence shown by SEQ ID NO:58 or a protein homologous thereto or a variant thereof and viloxazine or a derivative thereof capable of binding to the protein;
(b59) a combination of a protein comprising the amino acid sequence shown by SEQ ID NO:59 or a protein homologous thereto or a variant thereof and cefazolin, fenbufen, ketoprofen, colchicine, doxycycline, gabapentin, lidoflazine, probenecid or a derivative thereof capable of binding to the protein;
(b60) a combination of a protein comprising the amino acid sequence shown by SEQ ID NO:60 or a protein homologous thereto or a variant thereof and benzydamine, clenbuterol or a derivative thereof capable of binding to the protein;
(b61) a combination of a protein comprising the amino acid sequence shown by SEQ ID NO:61 or a protein homologous thereto or a variant thereof and benzethonium, fluphenazine, GBR12909, doxazosin, procaine, quinacrine or a derivative thereof capable of binding to the protein;
(b62) a combination of a protein comprising the amino acid sequence shown by SEQ ID NO:62 or a protein homologous thereto or a variant thereof and azithromycin, colistin or a derivative thereof capable of binding to the protein;
(b63) a combination of a protein comprising the amino acid sequence shown by SEQ ID NO:63 or a protein homologous thereto or a variant thereof and protriptyline, maprotiline or a derivative thereof capable of binding to the protein.
[7] A substance obtained by the method according to any one of [1] to [6] above.
[8] An agent of regulating a bioactivity, which comprises a substance obtained by the method according to any one of [1] to [6] above.
[9] An agent of regulating an action associated with a bioactive substance X, which comprises a substance that regulates the expression or function of a target protein Y or a gene that encodes the protein, wherein the combination of the bioactive substance X and the target protein Y is any combination of the combination A.
[10] The agent according to [9] above, wherein the substance that regulates the expression or function of a target protein Y or a gene that encodes the protein is a substance that suppresses the expression or function of the gene.
[11] The agent according to [10] above, wherein the substance that suppresses the expression or function of a target protein Y or a gene that encodes the protein is antisense nucleic acid, ribozyme, decoy nucleic acid, siRNA, antibody or dominant negative mutant, or an expression vector thereof.
[12] The agent according to [9] above, which comprises the target protein Y, or an expression vector comprising a nucleic acid that encodes the protein.
[13] An agent of regulating a function associated with a target protein Y, which comprises a bioactive substance X, wherein the combination of the target protein Y and the bioactive substance X is any combination of the combination B.
[14] A method of producing a derivative of bioactive substance X, which comprises derivatizing the bioactive substance X so as to be able to regulate the expression or function of a target protein Y or a gene that encodes the protein, wherein the combination of the bioactive substance X and the target protein Y is any combination of the combination A.
[15] A method of producing a derivative of a substance capable of regulating a function associated with a target protein Y, which comprises derivatizing a bioactive substance X so as to be able to regulate the ability of the bioactive substance X to bind to the target protein Y, wherein the combination of the target protein Y and the bioactive substance X is any combination of the combination B.
[16] A bioactive substance derivative obtained by the method according to [14] or [15] above.
[17] An agent of regulating a bioactivity, which comprises a bioactive substance derivative obtained by the method according to [14] or [15] above.
[18] A complex comprising a bioactive substance X and a target protein Y thereof, wherein the combination of the bioactive substance X and the target protein Y is any combination of the combination A or combination B.
[19] A method of producing the complex according to [18] above, which comprises bringing the bioactive substance and the target protein therefor into contact with each other.
[20] A kit comprising the following (i) and (ii):
(i) a bioactive substance X or a salt thereof;
(ii) a target protein Y, a nucleic acid that encodes the protein, an expression vector comprising the nucleic acid, cells that enable a measurement of the expression of the target protein Y or a gene that encodes the protein, or an expression vector comprising the transcription regulatory region of a gene that encodes the target protein Y and a reporter gene functionally linked thereto;

wherein the combination of the bioactive substance X and the target protein Y is any combination of the combination A or combination B.
[21] A method for determining the onset or risk of onset of a disease or condition associated with an action of a bioactive substance X, which comprises the following steps (a) and (b):
(a) a step for measuring the expression level and/or polymorphism of the target protein Y or a gene that encodes the protein in a biological sample collected from an animal;
(b) a step for evaluating the onset or likelihood of onset of the disease or condition on the basis of the measured expression level and/or polymorphism;

wherein the combination of the bioactive substance X and the target protein Y is any combination of the combination A.
[22] A method for determining the onset or risk of onset of a disease or condition associated with a function of a target protein Y, which comprises the following steps (a) and (b):
(a) a step for measuring the polymorphism of the gene that encodes the target protein Y in a biological sample collected from an animal;
(b) a step for evaluating the onset or likelihood of onset of the disease or condition on the basis of the presence or absence of a particular type of polymorphism;

wherein the particular type of polymorphism alters the ability of the target protein Y to bind to the bioactive substance X,

wherein the combination of the target protein Y and the bioactive substance X is any combination of the combination B. [23] A kit for determining the onset or risk of onset of a disease or condition associated with an action of a bioactive substance X, which comprises the following (i) and (ii):
(i) a means capable of measuring the expression level and/or polymorphism of a target protein Y or a gene that encodes the protein;
(ii) a medium recording the relationship between the disease or condition and the expression level and/or polymorphism of the gene;

wherein the combination of the bioactive substance X and the target protein Y is any combination of the combination A.
[24] A kit for determining the onset or risk of onset of a disease or condition associated with a function of a target protein Y, which comprises the following steps (i) and (ii):
(i) a means capable of measuring the polymorphism of a gene that encodes the target protein Y;
(ii) a medium recording the relationship between the disease or condition and the polymorphism of the gene;

wherein the particular type of polymorphism alters the ability of the target protein Y to bind to the bioactive substance X,

wherein the combination of the target protein Y and the bioactive substance X is any combination of the combination B.
[25] A method for determining susceptibility to a bioactive substance X in a disease or condition associated with an action of the bioactive substance X, which comprises the following steps (a) and (b):
(a) a step for measuring the expression level and/or polymorphism of a target protein Y or a gene that encodes the protein in a biological sample collected from an animal;
(b) a step for predicting the effect of the bioactive substance on the basis of the measured expression level and/or polymorphism;

wherein the combination of the bioactive substance X and the target protein Y is any combination of the combination A.
[26] A method for determining susceptibility to a bioactive substance X in a disease or condition associated with a function of a target protein Y, which comprises the following steps (a) and (b):
(a) a step for measuring the type of the polymorphism of the gene that encodes the target protein Y in a biological sample collected from an animal;
(b) a step for predicting the effect of the bioactive substance X in the disease or condition on the basis of the presence or absence of a particular type of polymorphism;

wherein the particular type of polymorphism alters the ability of the target protein Y to bind to the bioactive substance X,

wherein the combination of the target protein Y and the bioactive substance X is any combination of the combination B.
[27] A kit for determining susceptibility to a bioactive substance X in a disease or condition associated with an action of the bioactive substance X, which comprises the following (i) and (ii):
(i) a means capable of measuring the expression level and/or polymorphism of a gene that encodes the target protein Y;
(ii) a medium recording the relationship between the effect of the bioactive substance X and the expression level and/or polymorphism of the gene;

wherein the combination of the bioactive substance X and the target protein Y is any combination of the combination A.
[28] A kit for determining susceptibility to a bioactive substance X in a disease or condition associated with a function of a target protein Y, which comprises the following (i) and (ii):
(i) a means capable of identifying the polymorphism of a gene that encodes the target protein Y;
(ii) a medium recording the relationship between the effect of the bioactive substance X and a particular type of the polymorphism of the gene;

wherein the particular type of polymorphism alters the ability of the target protein Y to bind to the bioactive substance X,

wherein the combination of the target protein Y and the bioactive substance X is any combination of the combination B.
[29] A polynucleotide of any of the following (a) to (d):
(a) a polynucleotide consisting of the nucleotide sequence shown by SEQ ID NO: 64;
(b) a polynucleotide consisting of the nucleotide sequence shown by SEQ ID NO: 65;
(c) a polynucleotide consisting of a nucleotide sequence corresponding to the 606th-2363rd nucleotides of the nucleotide sequence shown by SEQ ID NO: 64; and
(d) a polynucleotide consisting of a nucleotide sequence corresponding to the 571st-1485th nucleotides of the nucleotide sequence shown by SEQ ID NO: 65.

### BRIEF DESCRIPTION OF THE DRAWINGS

FIG. 1 is a schematic diagram showing a SEC interaction screening system using a spin column.

FIG. 2 is a schematic diagram showing a SEC interaction analysis using a spin column.

### BEST MODE FOR CARRYING OUT THE INVENTION

### 1. Target Proteins and Target Genes for Bioactive Substances

The present invention provides target proteins and target genes for the development of bioactive substances.

A bioactive substance means any substance that has an action on the body. The bioactive substance can be an exogenous substance such as a drug, vitamin, herbal medicine ingredient, or food ingredient, and can be an endogenous substance such as a cytokine, growth factor, or hormone. When a given bioactive substance is intended, it is expressed as bioactive substance X as required.

Bioactive substance X includes the bioactive substances capable of regulating the expression or function of a target protein Y or a gene that encodes the protein, described below, for example, bioactive substances capable of binding to target protein Y. In detail, the bioactive substance X can be trimethylcolchicic acid, acenocoumarol, paracetamol, acetohexamide, acetopromazine, actinomycin D, ajmaline, albendazole, alfuzosin, a -methyl-5-hydroxytryptamine, amoxapine, antipyrine, azithromycin, benzbromarone, benzethonium, benzydamine, berberine, bezafibrate, bicartamide, boldine, bromperidol, budesonide, bupivacaine, buspirone, cefazolin, celestine blue, cephaeline, chlordiazepoxide, chlorogenic acid, chlorothiazide, chromomycin A3, ciclopirox, cisapride, clarithromycin, clemizole, clenbuterol, clobetasone, clofazimine, clofilium, clomiphene, clopamide, colchicine, colistin, conessine, coniine (DL), coralyne, cyclobenzaprine, cyclopentolate, cyclosporine A, diclofenac, dichlorphenamide, diflunisal, dihydrostreptomycin, diperodon, difenidol, dipyridamole, dizocilpine, DO897/99, domperidone, dopamine, doxazosin, doxycycline, eburnamonine, etodolac, fenbendazole, fenbufen, fenoprofen, flumequine, flupentixol, fluphenazine, fluvoxamine, furazolidone, gabapentin, GBR12909, glibenclamide, glipizide, gramicidin, guanfacine, harmol, hydroflumethiazide, hydroxychloroquine, hydroxytacrine(R,S), ifosfamide, iobenguane, iproniazid, isoxicam, isradipine, josamycin, ketoprofen, 3-hydroxykynurenine, leuprolide, L-thyroxine, lidoflazine, α-lobeline (−), loperamide, maprotiline, mebendazole, meclofenamic acid, metanephrine (D,L), metaproterenol, metergotamine, methimazole, methoxamine, methoxy-6-harmalan, mifepristone, minaprine, minocycline, misoprostol, molsidomine, moroxydine, moxalactam, mupirocin, nefopam, nicardipine, nimesulide, norharman, oxytocin, paroxetine, perhexiline, phenformin, pimethixene, piperlongumine, pirenzepine, probenecid, procaine, propranolol, protriptyline, pyrilamine, quercetin, quinacrine, quinine, rescinnamine, risperidone, ritodrine, saquinavir, scoulerine, sulfadimethoxine, sulfaphenazole, syrosingopine, tamoxifen, terconazole, thioproperazine, thiothixene(cis), tobramycin, tolbutamide, trifluoperazine, trimetazidine, viloxazine, xylazine, acetylsalicylsalicylic acid, nimetazepam, clobazam, alimemazine, tranilast, ebastine, pranlukast, methyclothiazide, alacepril, clinofibrate, acetylcysteine, buformin, terguride, stanozolol, mestanolone, pantethine, limaprost, sarpogrelate, argatroban, fludroxycortide, sulfadoxine, ubenimex, celecoxib, 6-furfurylaminopurine, solasodine, gossypol, fluorocurarine, pempidine, nitrarine, promazine, sulfabenzamide, althiazide, α-ergocryptine, ebselen, furaltadone, pyrithyldione, benzthiazide, levobunolol, raloxifene, luteolin, valdecoxib, carboprost, gabexate, or a derivative thereof capable of binding to target protein Y (described later), or a salt thereof.

Bioactive substances can also be roughly divided, from the viewpoint of the type of activity that can be regulated thereby, into two groups: substances capable of regulating an action associated with a bioactive substance X, and substances capable of regulating a function associated with a target protein Y.

The target proteins and target genes for the development of bioactive substances can preferably be target proteins and target genes for drug discovery. When a given target protein and a given target gene are intended, they are expressed as target protein Y and target gene Y, respectively, as required. The term protein has the same definition as a translation product, and the term target gene Y has the same definition as a gene that encodes target protein Y; these terms are interchangeably used.

For example, target protein Y can be a target protein for the above-described bioactive substance X. Specifically, target protein Y can be a protein comprising the amino acid sequence shown by SEQ ID NOs:1 to 63 (e.g., full-length protein) or a protein homologous thereto or a variant thereof. As mentioned herein, the target proteins of the present invention are not limited to human proteins, but include orthologues of different animal species. Referring to human proteins for reference, information on various aspects and some examples of binding bioactive substances discovered by the present inventors are shown in Tables 1-1 to 1-8 and Tables 2-1 to 2-20, respectively.

TABLE 1-1

| | Sequence | ORF | FLJ nucleotide | | | Example of bioactive |
|---|---|---|---|---|---|---|
| FLJ No. | No. | mutation | sequence Accession | H-InV cDNA ID | H-InV Locus ID | substances to be bound |
| FLJ21182 | 1 | — | AK024835.1 | HIT000008109.6 | HIX0014568.6 | trimethylcolchicic acid |
| FLJ21182 | 1 | — | AK024835.1 | HIT000008109.6 | HIX0014568.6 | ajmaline |
| FLJ21182 | 1 | — | AK024835.1 | HIT000008109.6 | HIX0014568.6 | antipyrine |
| FLJ21182 | 1 | — | AK024835.1 | HIT000008109.6 | HIX0014568.6 | boldine |
| FLJ21182 | 1 | — | AK024835.1 | HIT000008109.6 | HIX0014568.6 | celestine blue |
| FLJ21182 | 1 | — | AK024835.1 | HIT000008109.6 | HIX0014568.6 | cephaeline |
| FLJ21182 | 1 | — | AK024835.1 | HIT000008109.6 | HIX0014568.6 | ciclopirox |
| FLJ21182 | 1 | — | AK024835.1 | HIT000008109.6 | HIX0014568.6 | clofilium |
| FLJ21182 | 1 | — | AK024835.1 | HIT000008109.6 | HIX0014568.6 | conessine |
| FLJ21182 | 1 | — | AK024835.1 | HIT000008109.6 | HIX0014568.6 | coniine (DL) |
| FLJ21182 | 1 | — | AK024835.1 | HIT000008109.6 | HIX0014568.6 | difenidol |
| FLJ21182 | 1 | — | AK024835.1 | HIT000008109.6 | HIX0014568.6 | doxazosin |
| FLJ21182 | 1 | — | AK024835.1 | HIT000008109.6 | HIX0014568.6 | methoxy-6-harmalan |
| FLJ21182 | 1 | — | AK024835.1 | HIT000008109.6 | HIX0014568.6 | paroxetine |
| FLJ21182 | 1 | — | AK024835.1 | HIT000008109.6 | HIX0014568.6 | pimethixene |
| FLJ21182 | 1 | — | AK024835.1 | HIT000008109.6 | HIX0014568.6 | quinine |
| FLJ21182 | 1 | — | AK024835.1 | HIT000008109.6 | HIX0014568.6 | ritodrine |
| FLJ21182 | 1 | — | AK024835.1 | HIT000008109.6 | HIX0014568.6 | sulfadimethoxine |
| FLJ21182 | 1 | — | AK024835.1 | HIT000008109.6 | HIX0014568.6 | alimemazine |
| FLJ21182 | 1 | — | AK024835.1 | HIT000008109.6 | HIX0014568.6 | pantethine |
| FLJ38597 | 2 | — | AK095916.1 | HIT000020771.7 | HIX0016383.6 | trimethylcolchicic acid |
| FLJ38597 | 2 | — | AK095916.1 | HIT000020771.7 | HIX0016383.6 | ajmaline |
| FLJ38597 | 2 | — | AK095916.1 | HIT000020771.7 | HIX0016383.6 | celestine blue |
| FLJ38597 | 2 | — | AK095916.1 | HIT000020771.7 | HIX0016383.6 | methoxy-6-harmalan |
| FLJ38597 | 2 | — | AK095916.1 | HIT000020771.7 | HIX0016383.6 | minaprine |
| FLJ38597 | 2 | — | AK095916.1 | HIT000020771.7 | HIX0016383.6 | ritodrine |
| FLJ38597 | 2 | — | AK095916.1 | HIT000020771.7 | HIX0016383.6 | scoulerine |
| FLJ38597 | 2 | — | AK095916.1 | HIT000020771.7 | HIX0016383.6 | alimemazine |
| FLJ38597 | 2 | — | AK095916.1 | HIT000020771.7 | HIX0016383.6 | acetylcysteine |
| FLJ13700 | 3 | — | AK023762.1 | HIT000007036.6 | HIX0002055.6 | paracetamol |
| FLJ13700 | 3 | — | AK023762.1 | HIT000007036.6 | HIX0002055.6 | celestine blue |
| FLJ13700 | 3 | — | AK023762.1 | HIT000007036.6 | HIX0002055.6 | ciclopirox |

TABLE 1-2

| | | | | | | |
|---|---|---|---|---|---|---|
| FLJ13700 | 3 | — | AK023762.1 | HIT000007036.6 | HIX0002055.6 | coniine (DL) |
| FLJ13700 | 3 | — | AK023762.1 | HIT000007036.6 | HIX0002055.6 | tamoxifen |
| FLJ13700 | 3 | — | AK023762.1 | HIT000007036.6 | HIX0002055.6 | acetylcysteine |
| FLJ50683 | 4 | — | | | HIX0028362.4 | molsidomine |
| FLJ50199 | 5 | — | | | HIX0017082.7 | trimetazidine |
| FLJ26440 | 6 | — | AK129950.1 | HIT000049221.4 | HIX0025059.6 | αlobeline (—) |
| FLJ26440 | 6 | — | AK129950.1 | HIT000049221.4 | HIX0025059.6 | ebselen |
| FLJ21647 | 7 | — | AK025300.1 | HIT000008574.8 | HIX0014688.6 | moroxydine |
| FLJ26620 | 8 | — | AK130130.1 | HIT000049401.5 | HIX0002217.7 | xylazine |
| FLJ43792 | 9 | — | AK125780.1 | HIT000045653.4 | HIX0025047.5 | iobenguane |
| FLJ43792 | 9 | — | AK125780.1 | HIT000045653.4 | HIX0025047.5 | terguride |
| FLJ38127 | 10 | A787G: ATG(Met)GTG(Val) | AK095446.1 | HIT000020301.7 | HIX0005337.6 | eburnamonine |
| FLJ38127 | 10 | A787G: ATG(Met)GTG(Val) | AK095446.1 | HIT000020301.7 | HIX0005337.6 | quinine |
| FLJ38127 | 10 | A787G: ATG(Met)GTG(Val) | AK095446.1 | HIT000020301.7 | HIX0005337.6 | fluorocurarine |
| FLJ38127 | 10 | A787G: ATG(Met)GTG(Val) | AK095446.1 | HIT000020301.7 | HIX0005337.6 | furaltadone |
| FLJ35050 | 11 | — | AK092369.1 | HIT000017236.7 | HIX0012404.7 | hydroflumethiazide |
| FLJ27298 | 12 | — | AK130808.1 | HIT000050079.4 | HIX0003297.6 | methimazole |
| FLJ26262 | 13 | — | AK129773.1 | HIT000049044.4 | HIX0025019.4 | risperidone |
| FLJ90682 | 14 | — | AK075163.1 | HIT000082198.3 | HIX0025032.4 | bupivacaine |
| FLJ22923 | 15 | — | AK026576.1 | HIT000009850.7 | HIX0016413.7 | clofazimine |
| FLJ22923 | 15 | — | AK026576.1 | HIT000009850.7 | HIX0016413.7 | dipyridamole |
| FLJ22923 | 15 | — | AK026576.1 | HIT000009850.7 | HIX0016413.7 | loperamide |
| FLJ22871 | 16 | — | AK026524.1 | HIT000009798.6 | HIX0016521.6 | methyclothiazide |
| FLJ22871 | 16 | — | AK026524.1 | HIT000009798.6 | HIX0016521.6 | stanozolol |
| FLJ20398 | 17 | — | AK000405.1 | HIT000002880.7 | HIX0017158.8 | chromomycin A3 |
| FLJ20398 | 17 | — | AK000405.1 | HIT000002880.7 | HIX0017158.8 | meclofenamic acid |
| FLJ20398 | 17 | — | AK000405.1 | HIT000002880.7 | HIX0017158.8 | saquinavir |
| FLJ35377 | 18 | A531G: GAA(Glu)GAG(Glu) | AK092696.1 | HIT000017563.7 | HIX0012893.9 | pranlukast |
| FLJ35377 | 18 | A531G: GAA(Glu)GAG(Glu) | AK092696.1 | HIT000017563.7 | HIX0012893.9 | promazine |
| FLJ42145 | 19 | — | AK124139.1 | HIT000044012.4 | HIX0012893.9 | dihydrostreptomycin |
| FLJ42145 | 19 | — | AK124139.1 | HIT000044012.4 | HIX0012893.9 | iproniazid |
| FLJ42145 | 19 | — | AK124139.1 | HIT000044012.4 | HIX0012893.9 | nefopam |
| FLJ26144 | 20 | — | AK129655.1 | | | quercetin |
| FLJ26144 | 20 | — | AK129655.1 | | | pranlukast |

TABLE 1-3

| | | | | | | |
|---|---|---|---|---|---|---|
| FLJ26144 | 20 | — | AK129655.1 | | | luteolin |
| FLJ26374 | 21 | — | AK129884.1 | HIT000049155.4 | HIX0015008.7 | pranlukast |
| FLJ26371 | 22 | — | AK129881.1 | HIT000049152.4 | HIX0010481.7 | clemizole |
| FLJ26371 | 22 | — | AK129881.1 | HIT000049152.4 | HIX0010481.7 | fenbendazole |
| FLJ26371 | 22 | — | AK129881.1 | HIT000049152.4 | HIX0010481.7 | harmol |
| FLJ26371 | 22 | — | AK129881.1 | HIT000049152.4 | HIX0010481.7 | ifosfamide |
| FLJ26371 | 22 | — | AK129881.1 | HIT000049152.4 | HIX0010481.7 | piperlongumine |
| FLJ26371 | 22 | — | AK129881.1 | HIT000049152.4 | HIX0010481.7 | propranolol |
| FLJ45688 | 23 | — | AK127593.1 | HIT000047466.4 | HIX0001922.6 | acetohexamide |
| FLJ45688 | 23 | — | AK127593.1 | HIT000047466.4 | HIX0001922.6 | acetohexamide |
| FLJ45688 | 23 | — | AK127593.1 | HIT000047466.4 | HIX0001922.6 | benzethonium |
| FLJ45688 | 23 | — | AK127593.1 | HIT000047466.4 | HIX0001922.6 | clenbuterol |
| FLJ45688 | 23 | — | AK127593.1 | HIT000047466.4 | HIX0001922.6 | clomiphene |
| FLJ46688 | 23 | — | AK127593.1 | HIT000047466.4 | HIX0001922.6 | clopamide |
| FLJ45688 | 23 | — | AK127593.1 | HIT000047466.4 | HIX0001922.6 | cyclobenzaprine |
| FLJ45688 | 23 | — | AK127593.1 | HIT000047466.4 | HIX0001922.6 | etodolac |
| FLJ45688 | 23 | — | AK127593.1 | HIT000047466.4 | HIX0001922.6 | flupentixol |
| FLJ45688 | 23 | — | AK127593.1 | HIT000047466.4 | HIX0001922.6 | glibenclamide |
| FLJ45688 | 23 | — | AK127593.1 | HIT000047466.4 | HIX0001922.6 | glipizide |
| FLJ45688 | 23 | — | AK127593.1 | HIT000047466.4 | HIX0001922.6 | guanfacine |
| FLJ45688 | 23 | — | AK127593.1 | HIT000047466.4 | HIX0001922.6 | isoxicam |
| FLJ45688 | 23 | — | AK127593.1 | HIT000047466.4 | HIX0001922.6 | maprotiline |
| FLJ45688 | 23 | — | AK127593.1 | HIT000047466.4 | HIX0001922.6 | misoprostol |
| FLJ45688 | 23 | — | AK127593.1 | HIT000047466.4 | HIX0001922.6 | perhexiline |
| FLJ45688 | 23 | — | AK127593.1 | HIT000047466.4 | HIX0001922.6 | probenecid |
| FLJ45688 | 23 | — | AK127593.1 | HIT000047466.4 | HIX0001922.6 | sulfaphenazole |
| FLJ45688 | 23 | — | AK127593.1 | HIT000047486.4 | HIX0001922.6 | thioproperazine |
| FLJ45688 | 23 | — | AK127593.1 | HIT000047466.4 | HIX0001922.6 | thiothixene(cis) |
| FLJ45688 | 23 | — | AK127593.1 | HIT000047466.4 | HIX0001922.6 | tolbutamide |
| FLJ45688 | 23 | — | AK127693.1 | HIT000047466.4 | HIX0001922.6 | methyclothiazide |
| FLJ45688 | 23 | — | AK127593.1 | HIT000047466.4 | HIX0001922.6 | clinofibrate |
| FLJ45688 | 23 | — | AK127593.1 | HIT000047486.4 | HIX0001922.6 | argatroban |
| RLJ45688 | 23 | — | AK127593.1 | HIT000047466.4 | HIX0001922.6 | sulfadoxine |
| FLJ45688 | 23 | — | AK127593.1 | HIT000047466.4 | HIX0001922.6 | uberimex |

TABLE 1-4

| | | | | | | |
|---|---|---|---|---|---|---|
| FLJ45688 | 23 | — | AK127593.1 | HIT000047466.4 | HIX0001922.6 | celecoxib |
| FLJ45688 | 23 | — | AK127593.1 | HIT000047466.4 | HIX0001922.6 | gossypol |
| FLJ45688 | 23 | — | AK127593.1 | HIT000047466.4 | HIX0001922.6 | sulfabenzamide |
| FLJ45688 | 23 | — | AK127593.1 | HIT000047466.4 | HIX0001922.6 | althiazide |
| FLJ45688 | 23 | — | AK127593.1 | HIT000047466.4 | HIX0001922.6 | α-ergocryptine |
| FLJ45688 | 23 | — | AK127593.1 | HIT000047466.4 | HIX0001922.6 | benzthiazide |
| FLJ45688 | 23 | — | AK127593.1 | HIT000047466.4 | HIX0001922.6 | valdecoxib |
| FLJ45688 | 23 | — | AK127593.1 | HIT000047466.4 | HIX0001922.6 | gabexate |
| FLJ38820 | 24 | — | AK095939.1 | HIT000020794.8 | HIX0000427.8 | acetohexamide |
| FLJ38620 | 24 | — | AK095939.1 | HIT000020794.8 | HIX0000427.8 | isradipine |
| FLJ38620 | 24 | — | AK095939.1 | HIT000020794.8 | HIX0000427.8 | mupirocin |
| FLJ38620 | 24 | — | AK095939.1 | HIT000020794.8 | HIX0000427.8 | alacepril |
| FLJ38620 | 24 | — | AK095939.1 | HIT000020794.8 | HIX0000427.8 | limaprost |
| FLJ38620 | 24 | — | AK095939.1 | HIT000020794.8 | HIX0000427.8 | solasodine |
| FLJ38620 | 24 | — | AK095939.1 | HIT000020794.8 | HIX0000427.8 | carboprost |
| FLJ26267 | 25 | T287C: TTT(Phe)TCT(Ser) | AK129778.1 | HIT000049049.5 | HIX0006288.8 | dizocilpine |
| FLJ26267 | 25 | T287C: TTT(Phe)TCT(Ser) | AK129778.1 | HIT000049049.5 | HIX0006288.8 | fluvoxamine |
| FLJ26267 | 25 | T287C: TTT(Phe)TCT(Ser) | AK129778.1 | HIT000049049.5 | HIX0006288.8 | 3-hydroxykynurenine |
| FLJ26267 | 25 | T287C: TTT(Phe)TCT(Ser) | AK129778.1 | HIT000049049.5 | HIX0006288.8 | metergotamine |
| FLJ26267 | 25 | T287C: TTT(Phe)TCT(Ser) | AK129778.1 | HIT000049049.5 | HIX0006288.8 | methoxamine |
| FLJ26267 | 25 | T287C: TTT(Phe)TCT(Ser) | AK129778.1 | HIT000049049.5 | HIX0006288.8 | paroxetine |
| FLJ26267 | 25 | T287C: TTT(Phe)TCT(Ser) | AK129778.1 | HIT000049049.5 | HIX0006288.8 | nimetazepam |
| FLJ26267 | 25 | T287C: TTT(Phe)TCT(Ser) | AK129778.1 | HIT000049049.5 | HIX0006288.8 | fludroxycortide |
| FLJ26062 | 26 | — | AK129573.1 | HIT000048844.4 | HIX0005848.6 | fenoprofen |
| FLJ22936 | 27 | A209G: GAG(Glu)GGG(Gly) | AK026589.1 | HIT000009863.6 | HIX0017014.8 | acenocoumarol |
| FLJ22936 | 27 | A209G: GAG(Glu)GGG(Gly) | AK026589.1 | HIT000009863.6 | HIX0017014.8 | budesonide |
| FLJ22936 | 27 | A209G: GAG(Glu)GGG(Gly) | AK026589.1 | HIT000009863.6 | HIX0017014.8 | chlorogenic acid |
| FLJ22936 | 27 | A209G: GAG(Glu)GGG(Gly) | AK026589.1 | HIT000009863.6 | HIX0017014.8 | chlorothiazide |
| FLJ22936 | 27 | A209G: GAG(Glu)GGG(Gly) | AK026589.1 | HIT000009863.6 | HIX0017014.8 | diclofenac |
| FLJ22936 | 27 | A209G: GAG(Glu)GGG(Gly) | AK026589.1 | HIT000009863.6 | HIX0017014.8 | diperodon |
| FLJ22936 | 27 | A209G: GAG(Glu)GGG(Gly) | AK026589.1 | HIT000009863.6 | HIX0017014.8 | DO 897/99 |
| FLJ22936 | 27 | A209G: GAG(Glu)GGG(Gly) | AK026589.1 | HIT000009863.6 | HIX0017014.8 | nimesulide |
| FLJ22936 | 27 | A209G: GAG(Glu)GGG(Gly) | AK026589.1 | HIT000009863.6 | HIX0017014.8 | thioproperazine |
| FLJ22936 | 27 | A209G: GAG(Glu)GGG(Gly) | AK026589.1 | HIT000009863.6 | HIX0017014.8 | sarpogrelate |

TABLE 1-5

| | | | | | | |
|---|---|---|---|---|---|---|
| FLJ43223 | 28 | — | AK125213.1 | HIT000045086.5 | HIX0000381.7 | acetylsalicylsalicylic acid |
| FLJ26102 | 29 | A363C: AAA(Lys)AAC(Asn) | AK129613.1 | HIT000048884.4 | HIX0019559.8 | buspirone |
| FLJ25218 | 30 | — | AK057947.1 | HIT000014554.6 | HIX0010790.6 | α-methyl-5-hydroxytryptamine |
| FLJ25218 | 30 | — | AK057947.1 | HIT000014554.6 | HIX0010790.6 | dopamine |
| FLJ45675 | 31 | — | AK127580.1 | HIT000047453.4 | HIX0013592.8 | cisapride |
| FLJ25918 | 32 | — | AK098784.1 | HIT000023614.6 | HIX0012783.5 | berberine |
| FLJ25918 | 32 | — | AK098784.1 | HIT000023614.6 | HIX0012783.5 | celestine blue |
| FLJ25918 | 32 | — | AK098784.1 | HIT000023614.6 | HIX0012783.5 | diflunisal |
| FLJ25918 | 32 | — | AK098784.1 | HIT000023614.6 | HIX0012783.5 | mebendazole |
| FLJ25918 | 32 | — | AK098784.1 | HIT000023614.6 | HIX0012783.5 | tranilast |
| FLJ46709 | 33 | — | AK128550.1 | HIT000048423.4 | HIX0016132.7 | bromperidol |
| FLJ46709 | 33 | — | AK128550.1 | HIT000048423.4 | HIX0016132.7 | coralyne |
| RGNpc017 | 34 | — | BC006464.1 | HIT000053157.4 | HIX0011883.7 | acetohexamide |
| RGNpc017 | 34 | — | BC006464.1 | HIT000053157.4 | HIX0011883.7 | chromomycin A3 |
| RGNpc017 | 34 | — | BC006464.1 | HIT000053157.4 | HIX0011883.7 | DO 897/99 |
| RGNpc017 | 34 | — | BC006464.1 | HIT000053157.4 | HIX0011883.7 | domperidone |
| RGNpc017 | 34 | — | BC006464.1 | HIT000053157.4 | HIX0011883.7 | flupentixol |
| RGNpc017 | 34 | — | BC006464.1 | HIT000053157.4 | HIX0011883.7 | fluphenazine |
| RGNpc017 | 34 | — | BC006464.1 | HIT000053157.4 | HIX0011883.7 | L-thyroxine |
| RGNpc017 | 34 | — | BC006464.1 | HIT000053157.4 | HIX0011883.7 | trifluoperazine |
| RGNpc017 | 34 | — | BC006464.1 | HIT000053157.4 | HIX0011883.7 | clinofibrate |
| RGNpc017 | 34 | — | BC006464.1 | HIT000053157.4 | HIX0011883.7 | carboprost |
| FLJ40377 | 35 | — | AK097696.1 | HIT000022550.7 | HIX0015325.6 | alfuzosin |
| FLJ40377 | 35 | — | AK097696.1 | HIT000022550.7 | HIX0015325.6 | clobetasone |
| FLJ40377 | 35 | — | AK097696.1 | HIT000022550.7 | HIX0015325.6 | doxazosin |
| FLJ40377 | 35 | — | AK097696.1 | HIT000022550.7 | HIX0015325.6 | risperidone |
| FLJ40377 | 35 | — | AK097696.1 | HIT000022550.7 | HIX0015325.6 | pranlukast |
| FLJ25845 | 36 | — | AK098711.1 | HIT000023541.6 | HIX0023076.6 | acetopromazine |
| FLJ25845 | 36 | — | AK098711.1 | HIT000023541.6 | HIX0023076.6 | amoxapine |
| FLJ25845 | 36 | — | AK098711.1 | HIT000023541.6 | HIX0023076.6 | cephaeline |
| FLJ25845 | 36 | — | AK098711.1 | HIT000023541.6 | HIX0023076.6 | clenbuterol |
| FLJ25845 | 36 | — | AK098711.1 | HIT000023541.6 | HIX0023076.6 | cyclopentolate |
| FLJ25845 | 36 | — | AK098711.1 | HIT000023541.6 | HIX0023076.6 | domperidone |
| FLJ25845 | 36 | — | AK098711.1 | HIT000023541.6 | HIX0023076.6 | minocycline |

TABLE 1-6

| | | | | | | |
|---|---|---|---|---|---|---|
| FLJ25845 | 36 | — | AK098711.1 | HIT000023541.6 | HIX0023076.6 | moxalactam |
| FLJ25845 | 36 | — | AK098711.1 | HIT000023541.6 | HIX0023076.6 | perhexiline |
| FLJ25845 | 36 | — | AK098711.1 | HIT000023541.6 | HIX0023076.6 | phenformin |
| FLJ25845 | 36 | — | AK098711.1 | HIT000023541.6 | HIX0023076.6 | pyrilamine |
| FLJ25845 | 36 | — | AK098711.1 | HIT000023541.6 | HIX0023076.6 | terconazole |
| FLJ25845 | 36 | — | AK098711.1 | HIT000023541.6 | HIX0023076.6 | tobramycin |
| FLJ23662 | 37 | — | AK074242.1 | HIT000015022.8 | HIX0009561.7 | clofazimine |
| FLJ23662 | 37 | — | AK074242.1 | HIT000015022.8 | HIX0009561.7 | glibenclamide |
| FLJ23662 | 37 | — | AK074242.1 | HIT000015022.8 | HIX0009561.7 | raloxifene |
| FLJ12668 | 38 | — | AK022730.1 | HIT000006004.6 | HIX0012811.6 | albendazole |
| FLJ90085 | 39 | — | AK074566.1 | HIT000081601.3 | HIX0010664.6 | bezafibrate |
| FLJ90364 | 40 | T155C: GTC(Val)GCC(Ala) | AK074845.1 | HIT000081880.3 | HIX0004359.6 | pirenzepine |
| FLJ90401 | 41 | — | AK074882.1 | HIT000081917.3 | HIX0004441.5 | rescinnamine |
| FLJ25526 | 42 | — | AK098392.1 | HIT000023222.7 | HIX0004710.6 | benzbromarone |
| FLJ25526 | 42 | — | AK098392.1 | HIT000023222.7 | HIX0004710.6 | mifepristone |
| FLJ25526 | 42 | — | AK098392.1 | HIT000023222.7 | HIX0004710.6 | pranlukast |
| FLJ25526 | 42 | — | AK098392.1 | HIT000023222.7 | HIX0004710.6 | mestanolone |
| FLJ46896 | 43 | — | AK128871.1 | HIT000048744.5 | HIX0005417.9 | hydroxytacrine(R,S) |
| FLJ46896 | 43 | — | AK128871.1 | HIT000048744.5 | HIX0005417.9 | metaproterenol |
| FLJ46896 | 43 | — | AK128871.1 | HIT000048744.5 | HIX0005417.9 | metergotamine |
| FLJ46856 | 44 | — | AK128689.1 | HIT000048562.5 | HIX0002864.7 | eburnamonine |
| FLJ46856 | 44 | — | AK128689.1 | HIT000048562.5 | HIX0002864.7 | levobunolol |
| FLJ90345 | 45 | — | AK074826.1 | HIT000081861.3 | HIX0015240.6 | norharman |
| FLJ90345 | 45 | — | AK074826.1 | HIT000081861.3 | HIX0015240.6 | pyrilamine |
| FLJ26550 | 46 | — | AK130060.1 | | | celestine blue |
| FLJ26550 | 46 | — | AK130060.1 | | | nitrarine |
| FLJ90015 | 47 | — | AK074496.1 | HIT000081531.3 | HIX0004064.7 | clemizole |
| FLJ39454 | 48 | — | AK096773.1 | HIT000021628.8 | HIX0000029.9 | clobazam |
| FLJ45115 | 49 | — | AK127058.1 | HIT000046931.4 | HIX0021564.7 | clarithromycin |
| FLJ45115 | 49 | — | AK127058.1 | HIT000046931.4 | HIX0021564.7 | josamycin |
| FLJ45115 | 49 | — | AK127058.1 | HIT000046931.4 | HIX0021564.7 | oxytocin |
| FLJ90066 | 50 | G394A: GCC(Ala)ACC(Thr) | AK074547.1 | HIT000081582.3 | HIX0026144.4 | cyclosporine A |
| FLJ90066 | 50 | G394A: GCC(Ala)ACC(Tnr) | AK074547.1 | HIT000081582.3 | HIX0026144.4 | leuprolide |
| FLJ37995 | 51 | — | AK095314.1 | HIT000020169.7 | HIX0007627.7 | dichlorphenamide |

TABLE 1-7

| | | | | | | |
|---|---|---|---|---|---|---|
| FLJ37995 | 51 | — | AK095314.1 | HIT000020169.7 | HIX0007627.7 | benzthiazide |
| FLJ26058 | 52 | A754G: AAA(Lys)GAA(Glu) G763A: GCT(Ala)ACT(Thr) | AK129569.1 | HIT000048840.4 | HIX0020040.7 | furazolidone |
| FLJ26058 | 52 | A754G: AAA(Lys)GAA(Glu) G763A: GCT(Ala)ACT(Thr) | AK129569.1 | HIT000048840.4 | HIX0020040.7 | hydroxychloroquine |
| FLJ26058 | 52 | A754G: AAA(Lys)GAA(Glu) G763A: GCT(Ala)ACT(Thr) | AK129569.1 | HIT000048840.4 | HIX0020040.7 | metanephrine (D,L) |
| FLJ46369 | 53 | — | AK128235.1 | HIT000048108.5 | HIX0018303.8 | benzbromarone |
| FLJ46369 | 53 | — | AK128235.1 | HIT000048108.5 | HIX0018303.8 | benzethonium |
| FLJ46369 | 53 | — | AK128235.1 | HIT000048108.5 | HIX0018303.8 | bicartamide |
| FLJ46369 | 53 | — | AK128235.1 | HIT000048108.5 | HIX0018303.8 | clofazimine |
| FLJ46369 | 53 | — | AK128235.1 | HIT000048108.5 | HIX0018303.8 | domperidone |
| FLJ46369 | 53 | — | AK128235.1 | HIT000048108.5 | HIX0018303.8 | doxazosin |
| FLJ46369 | 53 | — | AK128235.1 | HIT000048108.5 | HIX0018303.8 | gramicidin |
| FLJ46369 | 53 | — | AK128235.1 | HIT000048108.5 | HIX0018303.8 | rescinnamine |
| FLJ46369 | 53 | — | AK128235.1 | HIT000048108.5 | HIX0018303.8 | saquinavir |
| FLJ46369 | 53 | — | AK128235.1 | HIT000048108.5 | HIX0018303.8 | syrosingopine |
| FLJ46369 | 53 | — | AK128235.1 | HIT000048108.5 | HIX0018303.8 | pranlukast |
| FLJ46369 | 53 | — | AK128235.1 | HIT000048108.5 | HIX0018303.8 | α-ergocryptine |
| FLJ16517 | 54 | — | AK131411.1 | HIT000249699.3 | HIX0032847.3 | actinomycin D |
| FLJ16517 | 54 | — | AK131411.1 | HIT000249699.3 | HIX0032847.3 | benzbromarone |
| FLJ16517 | 54 | — | AK131411.1 | HIT000249699.3 | HIX0032847.3 | clofazimine |
| FLJ16517 | 54 | — | AK131411.1 | HIT000249699.3 | HIX0032847.3 | domperidone |
| FLJ16517 | 54 | — | AK131411.1 | HIT000249699.3 | HIX0032847.3 | loperamide |
| FLJ16517 | 54 | — | AK131411.1 | HIT000249699.3 | HIX0032847.3 | nicardipine |
| FLJ16517 | 54 | — | AK131411.1 | HIT000249699.3 | HIX0032847.3 | quercetin |
| FLJ16517 | 54 | — | AK131411.1 | HIT000249699.3 | HIX0032847.3 | ebastine |
| FLJ16517 | 54 | — | AK131411.1 | HIT000249699.3 | HIX0032847.3 | pranlukast |
| FLJ16517 | 54 | — | AK131411.1 | HIT000249699.3 | HIX0032847.3 | luteolin |
| FLJ26591 | 55 | A442G: AGG(Arg)GGG(Gly) | AK130101.1 | HIT000049372.5 | HIX0006653.8 | pyrithyldione |
| FLJ26596 | 56 | C286A: CAG(Gln)AAG(Lys) | AK130106.1 | HIT000049377.4 | HIX0025206.4 | chlordiazepoxide |
| FLJ26596 | 56 | C286A: CAG(Gln)AAG(Lys) | AK130106.1 | HIT000049377.4 | HIX0025206.4 | flumequine |
| FLJ90480 | 57 | — | AK074961.1 | HIT000081996.3 | HIX0016009.9 | buformin |
| FLJ90480 | 57 | — | AK074961.1 | HIT000081996.3 | HIX0016009.9 | 6-furfurylaminopurine |
| FLJ90480 | 57 | — | AK074961.1 | HIT000081996.3 | HIX0016009.9 | pempidine |
| FLJ90480 | 57 | — | AK074961.1 | HIT000081996.3 | HIX0016009.9 | nitrarine |
| FLJ43067 | 58 | — | AK125057.1 | | | viloxazine |

TABLE 1-8

| | | | | | | |
|---|---|---|---|---|---|---|
| FLJ25460 | 59 | — | AK058189.1 | HIT000014795.6 | HIX0014594.8 | cefazolin |
| FLJ25460 | 59 | — | AK058189.1 | HIT000014795.6 | HIX0014594.8 | colchicine |
| FLJ25460 | 59 | — | AK058189.1 | HIT000014795.6 | HIX0014594.8 | doxycycline |
| FLJ25460 | 59 | — | AK058189.1 | HIT000014795.6 | HIX0014594.8 | fenbufen |
| FLJ25460 | 59 | — | AK058189.1 | HIT000014795.6 | HIX0014594.8 | gabapentin |
| FLJ25460 | 59 | — | AK058189.1 | HIT000014795.6 | HIX0014594.8 | ketoprofen |
| FLJ25460 | 59 | — | AK058189.1 | HIT000014795.6 | HIX0014594.8 | lidoflazine |
| FLJ25460 | 59 | — | AK058189.1 | HIT000014795.6 | HIX0014594.8 | probenecid |
| FLJ26806 | 60 | A237G: GTA(Val)GTG(Val) | AK130316.1 | HIT000049587.5 | HIX0002958.7 | benzydamine |
| FLJ26806 | 60 | A237G: GTA(Val)GTG(Val) | AK130316.1 | HIT000049587.5 | HIX0002958.7 | clenbuterol |
| FLJ43911 | 61 | — | AK125899.1 | HIT000045772.5 | HIX0027681.5 | benzethonium |
| FLJ43911 | 61 | — | AK125899.1 | HIT000045772.5 | HIX0027681.5 | doxazosin |
| FLJ43911 | 61 | — | AK125899.1 | HIT000045772.5 | HIX0027681.5 | fluphenazine |
| FLJ43911 | 61 | — | AK125899.1 | HIT000045772.5 | HIX0027681.5 | GBR12909 |
| FLJ43911 | 61 | — | AK125899.1 | HIT000045772.5 | HIX0027681.5 | procaine |
| FLJ43911 | 61 | — | AK125899.1 | HIT000045772.5 | HIX0027681.5 | quinacrine |
| FLJ44715 | 62 | — | AK126671.1 | HIT000046544.4 | HIX0008930.6 | azithromycin |
| FLJ44715 | 62 | — | AK126671.1 | HIT000046544.4 | HIX0008930.6 | colistin |
| FLJ90031 | 63 | — | AK074512.1 | | | maprotiline |
| FLJ90031 | 63 | — | AK074512.1 | | | protriptyline |

TABLE 2-1

| | Protein | Corresponding protein | | | |
|---|---|---|---|---|---|
| FLJ No. | name | variant | | function-activity | Cited reference |
| FLJ21182 | Calponin-2 | NM_004368.2 | NP_004359.1 | Actin-binding activity, | Mol Cell Biol. 1997 February; 17(2): 707-12.; Am J Physiol |
| | (Calponin | NM_201277.1 | NP_958434.1 | calmodulin binding activity, | Cell Physiol. 2003 January; 284(1): C156-67.; J Biochem |
| | H2, smooth | | | smooth muscle contraction | (Tokyo). 1996 August; 120(2): 415-24.; Genome Res. |
| | muscle) | | | control function, cell | 1996 September; 6(9): 791-806.; Nature. 2000 May |
| | (Neutral | | | skeleton organization and | 18; 405(6784): 311-9.; J Dermatol Sci. 1997 |
| | calponin). | | | biosynthesis control | January; 14(1): 29-36. |
| | | | | function, intercellular | |
| | | | | binding control function | |
| FLJ38597 | Smoothelin. | NM_134270.1 | NP_599032.1 | Actin-binding activity, | Proc Natl Acad Sci USA. 2004 Aug. 17; 101(33): 12130-5.; |
| | | NM_134269.1 | NP_599031.1 | muscle constituting factor, | J Mol Med. 1999 February; 77(2): 294-8.; FASEB J. |
| | | NM_006932.3 | NP_008863.3 | muscle differentiation | 2000 January; 14(1): 17-26.; Genomics. 1997 Jul. 15; |
| | | | | control function, smooth | 43(2): 245-7.; J Mol Med. 1999 February; 77 (2): |
| | | | | muscle contraction control | 255-7.; Cardiovasc Res. 2002 September; 55(4): 850-63.; |
| | | | | function, actin cell skeleton | J Vasc Res. 2001 March-April; 38(2): 120-32.; Cell Struct |
| | | | | constituting factor | Funct. 1997 February; 22(1): 65-72.; Histochem Cell Biol. |
| | | | | | 1999 October; 112(4): 291-9.; J Cell Biol. |
| | | | | | 1996 July; 134(2): 401-11. |

TABLE 2-2

| | | | | | |
|---|---|---|---|---|---|
| FLJ13700 | Spectrin beta | NM_003128.2 | NP_003119.2 | Actin-binding activity, | Genome Res. 2004 July; 14(7): 1324-32.; Proc Natl |
| | chain, brain 1 | NM_178313.2 | NP_842565.2 | cell skeleton | Acad Sci USA. 2004 Aug. 17; 101(33): 12130-5.; J |
| | (Spectrin, | | | constituting factor, | Mol Neurosci. 2001 August; 17(1): 59-70.; Nat Cell Biol. |
| | non-erythroid | | | calmodulin binding | 2004 February; 6(2): 97-105.; J Biol Chem. 2004 Sep. |
| | beta chain 1) | | | activity, SMAD protein | 17; 279(38): 40185-93.; Biochem J. 2001 Sep. |
| | (Beta-II | | | phosphorylation control, | 15; 358(Pt 3): 727-35.; J Neurochem. 1998 |
| | spectrin) | | | SMAD protein intranuclear | November; 71(5): 2220-8.; FEBS Lett. 1999 Jan. |
| | (Fodrin beta | | | transfer control, | 25; 443(2): 89-92; Science. 2003 Jan. |
| | chain). | | | cellular membrane | 24; 299(5606): 574-7.; J Proteome Res. 2005 July- |
| | | | | control factor | August; 4(4): 1339-46.; J Cell Sci. 2000 June; 113(Pt |
| | | | | | 11): 2023-34.; Neurobiol Dis. 2003 August; 13(3): 191- |
| | | | | | 202; J Biol Chem. 2003 Mar. 21; 278(12): 10048-54.; |
| | | | | | Oncogene. 2005 Mar. 10; 24(11): 1946-57.; Mol Cell |
| | | | | | Proteomics. 2004 November; 3(11): 1093-101.; Curr Biol. |
| | | | | | 2004 Aug. 24; 14(16): 1436-50.; Genome Res. 2004 |
| | | | | | September; 14(9): 1711-8.; J Biol Chem. 2001 Jun. |
| | | | | | 8; 276(23): 20679-87. |
| FLJ50683 | Plastin-3 | NM_005032.3 | NP_005023.2 | Actin-binding activity, | Cancer Res. 2003 Nov. 1; 63(21): 7122-7.; Cancer |
| | (T-plastin) | | | Ca ion binding activity, | Res. 1985 November; 45(11 Pt: 2): 5643-7.; J Cell Sci. |
| | | | | actin cell skeleton | 2005 Mar. 15; 118(Pt: 6): 1255-65.; Hum Mol Genet. |
| | | | | control function | 2005 Oct. 1; 14(19): 2893-909.; Reprod Biomed Online. |
| | | | | | 2003 September; 7(2): 235-42.; Mol Cell Biol. 1990 |
| | | | | | April; 10(4): 1818-21.; J Cell Biol. 1994 December; |
| | | | | | 127(6 Pt 2): 1995-2008.; J Biol Chem. 1993 Feb. |
| | | | | | 5; 268(4): 2781-92.; Mol Cell Biol. 1994 |
| | | | | | April; 14(4): 2457-67.; Mol Cell Biol. 1988 |
| | | | | | November; 8(11): 4659-68.; Int J Oncol. 2005 |
| | | | | | October; 27(4): 933-40. |

TABLE 2-3

| | | | | | |
|---|---|---|---|---|---|
| FLJ50199 | Rho guanine | NM_004840.2 | NP_004831.1 | Rho guanilnudeotide | Science. 2005 Mar. 11; 307(5715): 1621-5.; Proc Natl |
| | nudeotide | | | exchange factor activity, | Acad Sci USA. 2004 Aug. 17; 101(33): 12130-5.; J |
| | exchange | | | GTPase activator activity, | Biol Chem. 2005 Feb. 25; 280(8): 6879-89.; Oncogene. |
| | factor 6 | | | apoptosis control function, | 1999 Oct. 7; 18(41): 5680-90.; Am J Med Genet. |
| | (PAK- | | | JNK cascade control | 2001 Apr. 15; 100(1): 43-8.; Acta Neuropathol (Berl). |
| | interacting | | | function | 2006 January; 111(1): 29-38.; Hum Mol Genet. 2003 Jan. |
| | exchange | | | | 15; 12(2): 155-67.; Mol Cell Biol. 2001 |
| | factor alpha) | | | | October; 21(20): 6796-807.; J Med Genet. 1998 |
| | (Alpha-Pix) | | | | October; 35(10): 801-5.; Nat Genet. 2000 October; 26(2): |
| | (COOL-2). | | | | 247-50.; J Cell Physiol. 2006 November; 209(2): 568-79.; |
| | | | | | Curr Biol. 2004 Aug. 24; 14(16): 1436-50.; Nat. Methods. |
| | | | | | 2005 August; 2(8): 591-8.; Antioxid Redox Signal. 2004 |
| | | | | | August; 6(4): 713-20.; Anal Chem. 2004 May |
| | | | | | 15; 76(10): 2763-72.; FEBS Lett. 2003 Aug |
| | | | | | 28; 550(1-3): 119-23.; Curr Biol. 2005 Jan. |
| | | | | | 11; 15(1): 1-10.; J Biol Chem. 2000 Jul. |
| | | | | | 21; 275(29): 22373-80.; Genes Dev. 2002 Apr. |
| | | | | | 1; 16(7): 836-45. |
| FLJ26440 | Iodotyrosine | NM_203395.1 | NP_981932.1 | oxide reductase activity, | FASEB J. 2004 October; 18(13): 1574-6.; J Biol Chem. 2006 |
| | deiodinase | | | electron transfer function | Feb. 3; 281(5): 2812-9. |
| | (Iodotyrosine | | | | |
| | dehalogenase | | | | |
| | 1 precursor) | | | | |
| FLJ21647 | Ran-binding | NM_003624.1 | NP_003615.1 | Ran GTPase binding | Mol Cell Biol. 2003 December; 23(23): 8751-61.; FEBS |
| | protein 3 | NM_007320.1 | NP_015559.1 | activity, signal transduction | Lett. 1998 May 15; 427(3): 330-6.; J Cell Biol. 2001 |
| | (RanBP3). | NM_007322.1 | NP_015561.1 | function by small GTPase, | Jun. 25; 153(7): 1391-402.; J Biol Chem. 2002 May |
| | | | | protein intranuclear transfer | 17; 277(20): 17385-8. |
| | | | | control, nuclear pore | |
| | | | | passage control | |

TABLE 2-4

| | | | | | |
|---|---|---|---|---|---|
| FLJ26620 | Macrophage | NM_001747.2 | NP_001738.2 | Actin-binding activity, | J Biol Chem. 2003 Aug. 1; 278(31): 29136-44.; Mol Biol |
| | capping | | | protein complex formation | Cell. 2001 November; 12(11): 3527-37.; Cell. 1997 May |
| | protein | | | control function, response | 16; 89(4): 511-21.; J Biol Chem. 1995 Jan. |
| | (Actin- | | | control function to | 6; 270(1): 45-8.; J Cell Sci. 2004 Oct. 15; 117(Pt |
| | regulatory | | | exogeneous pathogen | 22): 5283-92.; J Biol Chem. 1992 Aug. |
| | protein | | | component, cell form | 15; 267(23): 16545-52.; Genomics. 1994 |
| | CAP-G). | | | control function, actin | October; 23(3): 560-5.; J Biol Chem. 2003 May |
| | | | | filament down arrow end | 16; 278(20): 17945-52. |
| | | | | capping function, cell | |
| | | | | skeleton formation control | |
| | | | | function, F-actin capping | |
| | | | | protein complex formation | |
| FLJ43792 | Guanylate | NM_000409.2 | NP_000400.2 | Ca ion binding activity, Ca | Hum Mol Genet. 1998 February; 7(2): 273-7.; Invest |
| | cyclase | | | sensitive guanylate cyclase | Ophthalmol Vis Sci. 2005 April; 46(4): 1124-32.; |
| | activating | | | activator activity, guanylate | Invest Ophthalmol Vis Sci. 2004 November; 45(11): 3863- |
| | protein 1 | | | cyclase control function, | 70.; Arch Ophthalmol. 2001 January; 119(1): 96-105.; |
| | (GCAP 1) | | | signal transduction function, | Mol Vis. 2005 Feb. 20; 11: 143-51.; Biochemistry. |
| | (Guanylate | | | vision control function, light | 2002 Oct. 29; 41(43): 13021-8.; J Biol Chem. 1998 |
| | cyclase | | | signal transduction function | Jul. 10; 273(28): 17311-4.; Biochemistry. 2004 Nov. |
| | activator | | | | 2; 43(43): 13796-804.; Mol Cell. 1998 July; 2(1): 129- |
| | 1A). | | | | 33.; Proc Natl Acad Sci USA. 2003 May |
| | | | | | 27; 100(11): 6783-8.; J Biol Chem. 1994 Dec. |
| | | | | | 9; 269(49): 31080-9.; Ophthalmology. 2005 |
| | | | | | August; 112(8): 1442-7.; Genomics. 1997 Feb. |
| | | | | | 1; 39(3): 312-22; Biochim Biophys Acta. 2002 Nov. |
| | | | | | 4; 1600(1-2): 111-7. |
| FLJ38127 | C5 orf3 | NM_018691.2 | NP_061161.2 | | Genome Res. 2006 January; 16(1): 55-65.; Genomics. |
| | (chromo- | | | | 2000 May 15; 66(1): 26-34. |
| | some | | | | |
| | 5 open | | | | |
| | reading | | | | |
| | frame 3) | | | | |

TABLE 2-5

| | | | | | |
|---|---|---|---|---|---|
| FLJ35050 | Pyruvate | NM_002654.3 | NP_002645.3 | Mg ion binding activity, | Genome Res. 2004 July; 14(7): 1315-23.; Anticancer |
| | kinase, | NM_182470.1 | NP_872270.1 | pyruvate kinase activity, | Res. 2003 March-April; 23(2A): 899-906.; Mol Cell |
| | isozyme | NM_182471.1 | NP_872271.1 | kinase function, transferase | Biochem. 2005 September; 277(1-2): 117-25.; Anticancer |
| | M1 (EC | | | activity, glycolytic system | Res. 2003 March-April; 23(2A): 851-3.; Genomics. |
| | 2.7.1.40) | | | control function, | 2003 February; 81(2): 112-25.; Anticancer Res. 2003 |
| | (Pyruvate | | | | March-April; 23(2A): 991-7.; J Struct Biol. 2000 |
| | kinase | | | | November; 132(2): 83-94.; J Proteome Res. 2005 May- |
| | muscle | | | | June; 4(3): 931-40.; Br J Nutr. 2002 January; 87 Suppl |
| | isozyme). | | | | 1: S23-9.; J Cell Sci. 2004 May 15; 117(Pt 12): 2557- |
| | | | | | 68.; Blood. 1998 Jul. 15; 92(2): 647-52.; |
| | | | | | Biochemistry. 2005 Jul. 12; 44(27): 9417-29.; J Biol |
| | | | | | Chem. 2002 Jun. 28; 277(26): 23807-14.; Mol Microbiol. |
| | | | | | 1998 January; 27(1); 171-86.; J Steroid Biochem Mol Biol. |
| | | | | | 2005 February; 94(1-3): 203-8. |

TABLE 2-6

| | | | | | |
|---|---|---|---|---|---|
| FLJ27298 | Trans- | NM_001664.2 | NP_001655.1 | Mg ion binding activity, | Cancer Res. 2006 Jan. 1; 66(1): 248-58.; Mol Biol |
| | forming | | | GTPase activity, signal | Cell. 2006 June; 17(6): 2489-97.; Methods Enzymol. |
| | protein | | | transduction activity, GTP | 2006; 406: 437-47.; Mol Biol Cell. 2006 |
| | RhoA | | | binding activity, cell | March; 17(3): 1204-17.; J Biol Chem. 2006 Sep. |
| | (H12). | | | adhesion control function, | 1; 281(35): 25089-96.; J Biol Chem. 2006 May |
| | | | | extracellular matrix control | 5; 281(18): 12908-18.; Mol Carcinog. 2006 |
| | | | | function, signal transduction | July; 45(7): 518-29.; Am J Physiol Lung Cell Mol |
| | | | | pathway via integrin-control | Physiol. 2006 June; 290(6): L1291-9.; Oncogene. |
| | | | | function, signal transduction | 2006 Sep. 28; 25(44): 5942-52.; Proc Natl Acad Sci USA. |
| | | | | control function by Small | 2006 Mar. 7; 103(10): 3639-44.; J Biol Chem. 2006 |
| | | | | GTPase, Rho protein signal | Apr. 14; 281(15): 10355-64.; J Biol Chem. 2006 Jun. |
| | | | | transduction control | 23; 281(25): 16951-61.; Biochem Biophys Res Commun. |
| | | | | function, muscle formation | 2006 Jun. 23: 345(1): 538-42.; Neurosci Lett. 2006 Oct. |
| | | | | control function, actin cell | 23; 407(2): 124-6.; J Biomed Sci. 2006 March; 13(2): 173- |
| | | | | skeleton organization and | 80.; Respir Res. 2006 Jun. 15; 7: 88.; Nat Cell Biol. |
| | | | | biosynthesis control, cell | 2006 May; 8(5): 485-91.; J Cell Biol. 2006 Jul. |
| | | | | differentiation control, NF-κB | 31; 174(3): 437-45.; J Appl Physiol. 2006 |
| | | | | intranuclear transfer positive | August; 101(2): 375-84.; Science. 2006 Jan. |
| | | | | control function, I-κB | 20; 311(5759): 377-81. |
| | | | | kinase/NF-κB cascade | |
| | | | | positive control function, | |
| | | | | stress · fiber formation | |
| | | | | control function | |

TABLE 2-7

| | | | | | |
|---|---|---|---|---|---|
| FLJ26262 | Chloride | NM_001288.4 | NP_001279.2 | potential dependent chlorine | J Neurosci. 1999 Apr. 15; 19(8): 2919-28.; J Biol |
| | intra- | | | ion channel activity, Ca ion | Chem. 2002 Oct. 25; 277(43): 40973-80.; Genomics. |
| | cellular | | | channel activity, ion | 2004 January; 83(1): 153-67.; J Biol Chem. 2001 Nov. |
| | channel | | | transport control function, | 30; 276(48): 44993-5000.; FASEB J. 2000 |
| | protein 1 | | | chlorine ion transport | June; 14(9): 1171-8.; J Immunol. 1999 Jul. |
| | (Nuclear | | | control, Ca ion transport | 1; 163(1): 278-87.; Genomics. 1997 Oct. 1; 45(1): 224- |
| | chloride | | | control | 8.; Mol Biol Cell. 2000 May; 11(5): 1509-21.; FEBS |
| | ion channel | | | | Lett. 2003 Apr. 10; 540(1-3): 77-80.; J Neurosci. |
| | 27) (NCC27) | | | | 2004 Jun. 9; 24(23): 5322-30.; Am J Physiol. 1998 |
| | (p64 CLCP) | | | | June; 274(6 Pt 2): F1140-9.; Biochem Biophys Res |
| | (Chloride | | | | Commun. 2005 Dec. 2; 337(4): 1308-18.; Am J Physiol |
| | channel | | | | Endocrinol Metab. 2005 September; 289(3): E419-28.; |
| | ABP). | | | | Proteomics. 2005 October; 5(15): 3876-84.; J Biol Chem. |
| | | | | | 2002 Jul. 19; 277(29): 26003-11.; Exp Eye Res. 2006 |
| | | | | | June; 82(6): 1046-52.; J Biol Chem. 2004 Mar. |
| | | | | | 5; 279(10): 9298-305.; J Physiol. 2000 Dec. 15; 529 Pt |
| | | | | | 3: 541-52. |
| FLJ90682 | Chloride | NM_016929.2 | NP_058625.2 | ion channel activity, | J Biol Chem. 2002 Oct. 25; 277(43): 40973-80.; |
| | intra- | | | potential dependent chlorine | Epilepsy Res. 2002 August; 50(3): 265-75.; Mol Biol Cell. |
| | cellular | | | ion channel activity, chlorine | 2000 May; 11(5): 1509-21.; DNA Res. |
| | channel | | | ion transporter activity, | 2005; 12(2): 117-26. |
| | protein 5. | | | AKAP350 binding activity, | |
| | | | | actin cell skeleton control of | |
| | | | | placental microvillus, | |
| | | | | pregnancy related function | |

TABLE 2-8

| | | | | | |
|---|---|---|---|---|---|
| FLJ22923 | Target | NM_005488.1 | NP_005479.1 | Intracellular protein | J Cell Sci. 2005 Feb. 1; 118(Pt 3): |
| | of Myb | | | transporter activity, golgi | 575-87.; J Biol Chem. 2004 Feb. 6; |
| | protein 1. | | | apparatus transport | 279(6): 4670-9.; Genome Res. 2003 |
| | | | | function, endocytosis | October; 13(10): 2265-70.; J Biol |
| | | | | control, endosome transport | Chem. 2004 Jun. 4; 279(23): 24435- |
| | | | | function, lysosome transport | 43.; Genomics. 1999 May 1; 57(3): |
| | | | | function, golgi apparatus | 380-8.; J Biol Chem. 2003 Dec. 26; |
| | | | | formation function | 278(52): 52865-72. |
| FLJ22871 | DNA- | NM_001018050.1 | NP_001018060.1 | nucleic acid binding | Mol Cell Biol. 2002 November; |
| | dependent | NM_001018051.1 | NP_001018061.1 | function, DNA dependent | 22(22): 8044-55.; DNA Res. 2001 |
| | RNA | NM_001018052.1 | NP_001018062.1 | RNA polymerase activity, | Feb. 28; 8(1): 1-9.; J Acquir |
| | polymerase III | NM_138338.2 | NP_612211.1 | iron ion binding activity, | Immune Defic Syndr. 1992; 5(11): |
| | subunit. | | | transferase activity, TCA | 1142-7. |
| | 22.9 kDa | | | cycle, citric acid metabolism, | |
| | polypeptide | | | transcription activity from | |
| | (EC 2.7.7.6) | | | RNA polymerase III | |
| | (RPC8). | | | promoter | |
| FLJ20398 | Ubiquitin- | NM_014235.2 | NP_055050.1 | protein post-translational | Proc Natl Acad Sci USA. 1988 |
| | like | | | modification, ubiquitin | February; 85(3): 851-5.; Gene |
| | protein 4 | | | modification reaction | Expr Patterns. 2007 January; |
| | (Ubiquitin- | | | | 7(1-2): 131-6. |
| | like | | | | |
| | protein | | | | |
| | GDX). | | | | |
| FLJ35377 | UBPH | NM_019116.2 | NP_061989.2 | | none |
| | ubiquitin- | | | | |
| | binding | | | | |
| | protein | | | | |
| | homolog | | | | |
| FLJ42145 | UBPH | NM_019116.2 | NP_061989.2 | | none |
| | ubiquitin- | | | | |
| | binding | | | | |
| | protein | | | | |
| | homolog | | | | |

TABLE 2-9

| FLJ26144 | Glucosamine- | NM_138335.1 | NP_612208.1 | glucosamine-6-phosphate | FEBS Lett. 2003 Sep. 11; 551(1-3): 63-70. |
|---|---|---|---|---|---|
| | 6-phosphate | | | deaminase activity, | |
| | isomerase | | | hydrocarbonate metabolism | |
| | (EC 3.5.99.6) | | | function, fructose 6 | |
| | (Glucosamine- | | | phosphate metabolism | |
| | 6-phosphate | | | control, glucosamine | |
| | deaminase) | | | metabolism control, N- | |
| | (GNPDA) | | | acetylglucosamine | |
| | (GlcN6P | | | metabolism control, | |
| | deaminase) | | | fertilization related | |
| | (Oscillin). | | | function, sperm acrosome | |
| | | | | reaction related function, | |
| | | | | fructose biosynthesis | |
| FLJ26374 | Glucose-6- | NM_000175.2 | NP_000166.2 | glucose 6 phosphate | J Rheumatol. 2004 August; 31(8): 1630-8.; Clin Cancer |
| | phosphate | | | isomerase activity, | Res. 2004 Nov. 15; 10(22): 7775-84.; Int J Cancer. |
| | isomerase | | | cytokine activity, growth | 2003 Dec. 10; 107(5): 707-14.; Blood Cells Mol Dis. |
| | (EC 5.3.1.9) | | | factor activity, | 2003 May-June; 30(3): 258-63.; Biochim Biophys Acta. |
| | (GPI) | | | hydrocarbonate | 2003 Feb. 21; 1645(2): 117-22.; J Biol Chem. 2005 |
| | (Phospho- | | | metabolism control, | Mar. 18; 280(11): 10419-26.; Nat Immunol. 2002 |
| | glucose | | | gluconeogenesis related, | April; 3(4): 366-72; Biochem Biophys Res Commun. |
| | isomerase) | | | glycolytic system related, | 2004 Oct. 15; 323(2): 518-22.; Nat Immunol. 2002 |
| | (PGI) | | | body humor immune | April; 3(4): 360-5.; Exp Hematol. 2005 May; 33(5): 531- |
| | (Phosphohexose | | | response, nerve | 41.; J Immunol. 2004 Apr. 1; 172(7): 4503-9.; |
| | isomerase) | | | development, hemostasis | Biochem Biophys Res Commun. 2004 Jan. |
| | (PHI) | | | | 30; 314(1): 76-82.; J Mol Biol. 2002 May |
| | (Neuroleukin) | | | | 10; 318(4): 385-97.; Cancer Res. 2004 Apr. |
| | (NLK) | | | | 1; 64(7): 2516-22.; Cancer Res. 2003 Jan. |
| | (Sperm | | | | 1; 63(1): 242-9.; Biochem Biophys Res Commun. |
| | antigen-36) | | | | 2006 Oct. 20; 349(2): 838-45.; J Biol Chem. 2003 Aug. |
| | (SA-36). | | | | 22; 278(34): 32165-72.; J Mol Biol. 2006 May |
| | | | | | 5; 358(3): 741-53.; FEBS Lett. 2003 Jan. 16; 534(1- |
| | | | | | 3): 49-53. |

TABLE 2-10

| | | | | | |
|---|---|---|---|---|---|
| FLJ26371 | L-lactate | NM_002300.3 | NP_002291.1 | lactate dehydrogenase | Ann Genet. 1975 June; 18(2): 81-7.; Biochem Biophys |
| | dehy- | | | activity, ATP binding activity, | Res Commun. 1990 Apr. 30; 168(2): 672-6.; Hum |
| | drogenase | | | oxide reductase activity, | Genet. 1993 June; 91(5): 423-6.; Clin Chim Acta. |
| | B chain (EC | | | anaerobic glycolytic system, | 1999 September; 287(1-2): 163-71.; FEBS Lett. 1992 |
| | 1.1.1.27) | | | TCA cycle intermediate | Mar. 16; 299(3): 231-4.; Breast Cancer Res Treat. |
| | (LDH-B) | | | metabolism | 2002 June; 73(3): 245-56.; Hum Genet. 1992 |
| | (LDH heart | | | | May; 89(2): 158-62.; Biochem Biophys Res Commun. |
| | subunit) | | | | 2005 Dec. 2; 337(4): 1308-18.; Proteomics. 2005 |
| | (LDH-H). | | | | October; 5(15): 3876-84.; Proteins. 2001 May |
| | | | | | 1; 43(2): 175-85.; Biochem J. 1989 Feb. 1; 257(3): |
| | | | | | 921-4.; Biochem J. 1987 Dec. 15; 248(3): 933-6. |
| FLJ45688 | Protein | NM_177983.1 | NP_817092.1 | Mg ion binding activity, Mn | J Mol Biol. 2006 Feb. 10; 356(1): 111-20.; Mol Cell |
| | phosphatase | NM_002707.3 | NP_002698.1 | ion binding activity, | Biol. 1997 September; 17(9): 5485-98.; FEBS Lett. |
| | 2C gamma | | | phosphatase activity to | 1997 Aug. 4; 412(3): 415-9.; Proc Natl Acad Sci |
| | isoform (EC | | | phosphorylated protein, | USA. 2003 Dec. 23; 100(26): 16006-11.; Genes Dev. |
| | 3.1.3.1 6) | | | serine/treonine type protein | 1999 Jan. 1; 13(1): 87-97. |
| | (PP2C-gamma) | | | phosphatase activity, | |
| | (Protein | | | dephosphorylation reaction | |
| | phosphatase | | | control activity, protein | |
| | magnesium- | | | phosphatase 2C activity, | |
| | dependent 1 | | | cell cycle control function | |
| | gamma) | | | | |
| | (Protein | | | | |
| | phosphatase | | | | |
| | 1C). | | | | |
| FLJ38620 | RPRC1 | NM_018067.3 | NP_060537.3 | cell skeleton control protein | Hum Genet. 1998 December; 103(6): 666-73.; |
| | arginine/ | | | binding activity, microtubule | DNA Res. 1999 Oct. 29; 6(5): 329-36. |
| | proline | | | control function, microtubule | |
| | rich | | | binding complex | |
| | coiled- | | | | |
| | coil 1 | | | | |

TABLE 2-11

| FLJ2627 | Protein-L-isoaspartate | NM_005389.1 | NP_005380.1 | protein-L-isoaspartate (D- | Mol Genet. Metab. 2006 January; |
|---|---|---|---|---|---|
| | (D-aspartate) O- | | | aspartate) O- | 87(1): 66-70.; Biochem Biophys |
| | methyltransferase | | | methyltransferase activity, | Res Commun. 1992 May 29; 185(1): |
| | (EC 2.1.1.77) | | | methyltransferase activity, | 277-83.; Biochem Biophys Res |
| | (Protein-beta- | | | S-adenosyl methionine | Commun. 1994 Aug. 30; 203(1): |
| | aspartate | | | dependent | 491-7.; Cytogenet. Cell Genet. |
| | methyltransferase) | | | methyltransferase activity, | 1999; 84(1-2): 130-1.; J Biochem |
| | (PIMT) (Protein L- | | | protein modification, protein | (Tokyo). 1995 April; 117(4): 683- |
| | isoaspartyl/D-aspartyl | | | amino acid residue | 5.; J Biol Chem. 2002 Mar. 22; |
| | methyltransferase) | | | methylation control | 277(12): 10642-6.; Protein Sci. |
| | (L-isoaspartyl protein | | | | 2002 March; 11(3): 625-35.; |
| | carboxyl | | | | Biochem Biophys Res Commun. 2003 |
| | methyltransferase). | | | | Sep. 12; 309(1): 44-51.; J Biol |
| | | | | | Chem. 2002 May 31; 277(22): 20011- |
| | | | | | 9.; Genomics. 1992 December; 14(4): |
| | | | | | 852-6.; Biochem Biophys Res Commun. |
| | | | | | 1988 Mar. 30; 151(3): 1136-43. |
| FLJ26062 | Lactoylglutathione | NM_006708.1 | NP_006699.1 | lactoyl glutathionelyase | Genetika. 2003 July; 39(7): 996- |
| | lyase (EC 4.4.1.5) | | | activity | 1002.; Neurobiol Aging. 2006 June; |
| | (Methylglyoxalase) | | | | 27(6): 815-22; Neurosci Lett. 2006 |
| | (Aldoketomutase) | | | | Mar. 27; 396(2): 163-6.; J Biol |
| | (Glyoxalase I) | | | | Chem. 1993 Mar. 15; 268(8): 5661-7.; |
| | (Glx I) (Ketone- | | | | Genome Res. 2006 January; 16(1): |
| | aldehyde mutase) | | | | 55-65.; Gene. 1999 Nov. 15; 240(1): |
| | (S-D-lactoylglutathione | | | | 149-55.; Blood. 2000 May 15; 95(10): |
| | methylglyoxal lyase). | | | | 3214-8.; J Biol Chem. 1993 May 25; |
| | | | | | 268(15): 11217-21.; J Biol Chem. |
| | | | | | 1998 Aug. 21; 273(34): 21623-8.; |
| | | | | | Genomics. 1991 December; 11(4): |
| | | | | | 875-84.; Chem Biol Interact. 2003 |
| | | | | | Feb. 1; 143-144: 341-51.; Biochem |
| | | | | | J. 1996 Mar. 1; 314 (Pt 2): 463-7.; |
| | | | | | Cancer J. 2006 May-June; 12(3): |
| | | | | | 222-8.; J Neurosci Res. 2006 June; |
| | | | | | 83(8): 1591-600.; Proteomics. 2005 |
| | | | | | October; 5(15): 3876-84.; Prep |
| | | | | | Biochem Biotechnol. 2001 August; |
| | | | | | 31(3): 305-16.; Clin Cancer Res. |
| | | | | | 2001 August; 7(8): 2513-8.; Mech |
| | | | | | Ageing Dev. 1998 Mar. 16; 101(1-2): |
| | | | | | 101-10.; J Infect. 1992 May; 24(3): |
| | | | | | 317-20. |

TABLE 2-12

| | | | | | |
|---|---|---|---|---|---|
| FLJ22936 | Septin 6. | NM_145799.2 | NP_665798.1 | GTP bond, protein bond, | J Biol Chem. 2003 Jan. 31; 278(5): 3483-8.; J Comp |
| | | NM_015129.4 | NP_055944.2 | cytoplasm division control | Neurol. 2000 Dec. 11; 428(2): 223-39.; Dokl Biochem |
| | | NM_145800.2 | NP_665799.1 | function, cell cycle control | Biophys. 2003 July-August; 391: 195-7.; Oncogene. 2002 |
| | | NM_145802.2 | NP_665801.1 | function | Jul. 11; 21(30): 4706-14.; J Biol Chem. 2000 Apr. |
| | | | | | 7; 275(14): 10047-56.; DNA Res. 1995 Aug. 31; 2(4): |
| | | | | | 167-74, 199-210.; Cancer Res. 2002 Jan. 15; 62(2): |
| | | | | | 333-7.; J Biol Chem. 2006 Oct. 13; 281(41): 30697-706.; |
| | | | | | Mol Biol Cell. 2002 October; 13(10): 3532-45.; |
| | | | | | Neuroreport. 2003 Jan. 20; 14(1): 31-7. |
| FLJ43223 | Tyrosyl- | NM_003680.2 | NP_003671.1 | tRNA binding activity, RNA | Biochemistry. 2002 Nov. 12; 41(45): 13344-9.; J Biol |
| | tRNA | | | binding activity, tyrosine- | Chem. 2002 Apr. 26; 277(17): 14812-20.; J Biol Chem. |
| | synthetase, | | | tRNA ligase activity, signal | 2002 Aug. 9; 277(32): 28394-9.; Proc Natl Acad Sci USA. |
| | cytoplasmic | | | transduction substance | 2002 Nov. 26; 99(24): 15369-74.; Nat Genet. 2006 |
| | (EC 6.1.1.1) | | | function, cytokine activity, | February; 38(2): 197-202; Am J Hum Genet. 2003 |
| | (Tyrosyl- | | | IL-8 receptor binding | December; 73(6): 1423-30.; Proc Natl Acad Sci USA. 1996 |
| | tRNA ligase) | | | activity, ATP binding | Jan. 9; 93(1): 166-70.; Protein Expr Purif. 2003 |
| | (TyrRS). | | | activity, protein bio- | January; 27(1): 104-8.; EMBO J. 1998 Jan. 2; 17(1): 297- |
| | | | | synthesis control, tRNA | 305.; J Biol Chem. 1999 Aug. 13; 274(33): 23155- |
| | | | | aminoacylation reaction | 9.; RNA. 2005 May; 11(5): 558-62.; J Biol Chem. |
| | | | | control in protein transla- | 1997 May 30; 272(22): 14420-5.; J Biol Chem. 2002 |
| | | | | tion, apoptosis control, | Jun. 7; 277(23): 20124-6.; J Biol Chem. 2002 Jun. |
| | | | | cellular motility control | 7; 277(23): 20243-8.; Biochemistry. 2005 Mar. 29; 44(12): |
| | | | | function | 4805-16.; Science. 1999 Apr. 2; 284(5411): 147-51. |

TABLE 2-13

| | | | | | |
|---|---|---|---|---|---|
| FLJ26102 | High-affinity | NM_001859.2 | NP_001850.1 | Copper ion | J Biol Chem. 2002 Jul. 19; 277(29): 26021-30.; |
| | copper uptake | | | transporter | Biochem J. 2002 Jun. 1; 364(Pt 2): 497-505.; J Biol |
| | protein 1 | | | activity, | Chem. 2002 Feb. 8; 277(6): 4380-7.; J Biol Chem. |
| | (hCTR1) (Copper | | | ion carrier | 2002 Oct. 25; 277(43): 40253-9.; Gene. 2000 Oct. |
| | transporter 1) | | | activity, | 17; 257(1): 13-22.; J Biol Chem. 2004 Nov. |
| | (Solute | | | copper ion | 5; 279(45): 46393-9.; J Biol Chem. 2003 Mar. |
| | carrier family | | | transport | 14; 278(1): 9639-46.; Placenta. 2006 September- |
| | 31, member 1). | | | function | October; 27(9-10): 968-77.; Proc Natl Acad Sci |
| | | | | | USA. 1997 Jul. 8; 94(14): 7481-6.; J Biol |
| | | | | | Chem. 2004 Apr. 23; 279(17): 17428-33.; J Biol |
| | | | | | Chem. 2002 Aug. 9; 277(32): 29162-71.; Proc Natl |
| | | | | | Acad Sci USA. 2006 Mar. 7; 103(10): 3627-32.; |
| | | | | | J Biol Chem. 2005 Mar. 11; 280(10): 9635-9.; |
| | | | | | Biochem J. 2003 Mar. 15; 370(Pt 3): 881-9. |
| FLJ25218 | MGC14817 | NM_032338.2 | NP_115714.1 | | Nature. 2005 Oct. 20; 437(7062): 1173-8. |
| FLJ45675 | C17orf39 chromo- | NM_024052.4 | NP_076957.3 | | Genome Res. 2002 May; 12(5): 713-28. |
| | some 17 open | | | | |
| | reading frame 39 | | | | |
| FLJ25918 | HSCARG protein; | NM_020677.2 | NP_065728.1 | | none |
| | NmrA-like family | | | | |
| | domain containing 1 | | | | |
| FLJ46709 | Protein C21 orf25 | NM_199050.1 | NP_950251.1 | | Int J Oncol. 2004 September; 25(3): 759-64. |
| | precursor; TMEM24 | | | | |
| | (Transmembrane | | | | |
| | protein 24; | | | | |
| | DLNB23 protein)- | | | | |
| | like(TMEM24L) | | | | |

TABLE 2-14

| | | | | | |
|---|---|---|---|---|---|
| RGNpc017 | Calmodulin | NM_006888.3 | NP_008819.1 | | Circ Res. 2006 May 26; 98(10): 1273-81.; Mol |
| | | | | | Pharmacol. 2006 February; 69(2): 608-17.; Hum Mol Genet. |
| | | | | | 2005 Apr. 15; 14(8): 1009-17.; J Biol Chem. 2005 Sep. |
| | | | | | 16; 280(37): 32426-33.; J Biol Chem. 2005 Oct. 28; |
| | | | | | 280(43): 35967-73.; FEBS Lett. 2005 Jan. 31; 579(3): |
| | | | | | 803-7.; Exp Cell Res. 2005 Nov. 1; 310(2): 293-302; |
| | | | | | Biochem Biophys Res Commun. 2005 Sep. 23; 335(2): 424-31.; |
| | | | | | EMBO J. 2005 Jun. 15; 24(12): 2104-13.; Mol Endocrinol. |
| | | | | | 2005 July; 19(7): 1884-92.; Genome Res. 2006 January; |
| | | | | | 16(1): 55-65.; Chem Biol. 2005 January; 12(1): 89-97.; |
| | | | | | Nat Struct Mol Biol. 2005 December; 12(12): 1108-15.; |
| | | | | | Biopolymers. 2005 Dec. 5; 79(5): 231-7.; J Physiol. 2005 |
| | | | | | Jun. 1; 565(Pt 2): 349-70.; Protein Sci. 2005 February; |
| | | | | | 14(2): 494-503.; Epub 2005 Apr. 7.; J Biol Chem. 2005 |
| | | | | | Feb. 25; 280(8): 7070-9.; Oncogene. 2005 Jun. 16; 24(26): |
| | | | | | 4206-19.; J Biol Chem. 2006 Jun. 23; 281(25): 17379-89. |
| FLJ40377 | hypothetical protein | NM_144688.3 | NP_653289.3 | | Nature. 2005 Oct. 20; 437(7062): 1173-8. |
| | FLJ32658 (highly | | | | |
| | similar to dual | | | | |
| | specificity protein | | | | |
| | phosphatase 8) | | | | |
| FLJ25845 | armadillo repeat | NM_173081.1 | NP_775104.1 | | Clin Cancer Res. 2006 Jan. 1; 12(1): 191-7.; Genetika. |
| | containing 3 | | | | 2006 July; 42(7): 999-1003. |
| FLJ23662 | tripartite motif | NM_017583.3 | NP_060053.2 | Znion | Brain Res Mol Brain Res. 2001 Jan. 31; 86(1-2): 153- |
| | protein 44 (DIPB | | | binding | 67.; EMBO J. 2001 May 1; 20(9): 2140-51. |
| | protein). | | | activity | |
| FLJ12668 | activating tran- | NM_024997.2 | NP_079273.2 | | J Biol Chem. 2005 Apr. 8; 280(14): 13928-35. |
| | scription factor 7 | | | | |
| | interacting protein 2 | | | | |

TABLE 2-15

| | | | | | |
|---|---|---|---|---|---|
| FLJ90085 | SPRY domain | NM_032840.1 | NP_116229.1 | kinase activity, protein | none |
| | containing 3 | | | tyrosine kinase activity, | |
| | | | | receptor activity | |
| FLJ90364 | ADP-ribose | NM_024047.3 | NP_076952.1 | Mg ion binding activity, Mn | Nature. 2001 May 31; 411(6837): |
| | pyrophosphatase, | NM_198038.1 | NP_932155.1 | ion binding activity, Ca ion | 595-9.; Biochim Biophys Acta. |
| | mitochondrial | | | activation cation channel | 2002 Jan. 31; 1594(1): 127-35.; |
| | precursor | | | activity, hydrolase activity, | J Biol Chem. 2003 Jan. 17; 278(3): |
| | (EC 3.6.1.13) | | | ADP-sugar diphosphatase | 1794-801.; J Mol Biol. 2003 Sep. |
| | (ADP-ribose | | | activity, ADP-ribose | 12; 332(2): 385-98.; Genome Res. |
| | diphosphatase) | | | diphosphatase activity, | 2003 October; 13(10): 2265-70.; |
| | (Adenosine | | | cation transport function | Biochim Biophys Acta. 2006 |
| | diphosphoribose | | | | October; 1760(10): 1545-51. |
| | pyrophosphatase) | | | | |
| | (ADPR-PPase) | | | | |
| | (ADP-ribose | | | | |
| | phosphohydrase) | | | | |
| | (Nucleoside | | | | |
| | diphosphate-linked | | | | |
| | moiety X motif 9) | | | | |
| | (UNQ3012/PRO9771). | | | | |
| FLJ90401 | ELOVL family | NM_024090.1 | NP_076995.1 | fatty acid elongation | J Biol Chem. 2001 Nov. 30; |
| | member 6, | | | enzyme activity, | 276(48): 45358-66. |
| | elongation | | | transferase activity, | |
| | of long chain | | | fatty acid elongation | |
| | fatty acids | | | reaction control | |
| FLJ25526 | Tubulin poly- | NM_007030.1 | NP_008961.1 | | J Hum Genet. 1999; 44(2): 121-2; |
| | merization- | | | | J Cell Sci. 2004 Dec. 1; 117(Pt |
| | promoting protein | | | | 25): 6249-59.; Biochem Biophys |
| | (TPPP) (25 kDa | | | | Res Commun. 2006 Jun. 23; 345(1): |
| | brain-specific | | | | 324-31.; Biochim Biophys Acta. |
| | protein) | | | | 2002 Jan. 2; 1586(1): 113-22.; |
| | (glycogen synthase | | | | J Biol Chem. 2005 Feb. 18; 280(7): |
| | kinase 3 (GSK3) | | | | 5703-15.; J Neurochem. 2006 |
| | inhibitor p24) | | | | October; 99(1): 333-42.; Proc Natl |
| | | | | | Acad Sci USA. 2003 Nov. 25; |
| | | | | | 100(24): 13976-81. |
| FLJ46896 | SH3PXD2B | NM_001017995.1 | NP_001017995.1 | Intracellular signal | DNA Res. 2000 Feb. 28; 7(1): |
| | SH3 and | | | transduction cascade | 65-73. |
| | PX domains 2B | | | control, protein | |
| | | | | transport function | |

TABLE 2-16

| | | | | | |
|---|---|---|---|---|---|
| FLJ46856 | Striated muscle | XM_001131579.1 | XP_001131579.1 | protein serine/treonine | J Biol Chem. 1996 Jul. 19; 271(29): |
| | preferentially | | | kinase activity, protein | 17354-9.; J Biol Chem. 1999 May 14; |
| | expressed | | | tyrosine kinase activity, ATP | 274(20): 14344-51.; J Cell Mol Med. |
| | protein kinase | | | binding activity, kinase | 2005 January-March; 9(1): 153-9.; |
| | (Aortic | | | activity, transferase activity, | Dev Genes Evol. 2004 July; 214(7): |
| | preferentially | | | protein phosphorylation | 352-9.; DNA Res. 2000 Feb. 28; 7(1): |
| | expressed protein | | | control, muscle | 65-73.; Genomics. 2006 June; 87(6): |
| | 1) (APEG-1) | | | differentiation, cell | 733-46.; BMC Struct Biol. 2005 Dec. |
| | | | | proliferation negative control | 14; 5: 21. |
| | | | | function | |
| FLJ90345 | Homeobox protein | NM_175875.3 | NP_787071.2 | DNA binding activity, | Hum Mol Genet. 1995 October; 4(10): |
| | SIX5 (DM locus- | | | transcription factor activity, | 1919-25.; Cell. 2006 May 19; 125(4): |
| | associated homeo- | | | transcription control, | 801-14.; Hum Mol Genet. 1999 March; |
| | domain protein). | | | differentiation control, | 8(3): 481-92; Mol Cell Biol. 1999 |
| | | | | transcription factorcomplex | October; 19(10): 6815-24.; J Clin |
| | | | | formation control | Pathol. 2000 March; 53(3): 212-7.; |
| | | | | | Nucleic Acids Res. 2000 May 1; 28(9): |
| | | | | | 1871-8.; J Biol Chem. 2002 Mar. 1; |
| | | | | | 277(9): 7021-8.; Hum Mol Genet. 2002 |
| | | | | | May 1; 11(9): 1045-58.; Hum Mol Genet. |
| | | | | | 1998 December; 7(13): 2103-12. |
| FLJ26550 | Transaldolase | NM_006755.1 | NP_006746.1 | Transaldolase activity, | Genome Res. 2004 July; 14(7): 1315- |
| | (EC 2.2.1.2). | | | transferase activity, | 23.; Mol Cell. 2004 Sep. 24; 15(6): |
| | | | | hydrocarbonate | 853-65.; Gene. 1998 Mar. 16: 209(1-2): |
| | | | | metabolism, pentose | 13-21.; J Biol Chem. 1994 Jan. 28; |
| | | | | phosphate pathway control | 269(4): 2847-51.; J Biol Chem. 2000 |
| | | | | | Mar. 10; 275(10): 7261-72.; Genomics. |
| | | | | | 1997 Mar. 1; 40(2): 378-81.; J Exp |
| | | | | | Med. 1994 Nov. 1; 180(5): 1649-63.; |
| | | | | | Proteomics. 2005 October; 5(15): |
| | | | | | 3876-84.; Genomics. 1997 Oct. 1; |
| | | | | | 45(1): 233-8.; FEBS Lett. 2000 Jun. |
| | | | | | 23; 475(3): 205-8.; Am J Hum Genet |
| | | | | | 2001 May; 68(5): 1086-92.; Metabolism. |
| | | | | | 2005 August; 54(8): 1027-33.; J Biol |
| | | | | | Chem. 2004 Mar. 26; 279(13): 12190-205. |

TABLE 2-17

| | | | | | |
|---|---|---|---|---|---|
| FLJ90015 | Mof4 family | NM_033296.1 | NP_150638.1 | protein | J Biol Chem. 2001 Oct. 19; 276(42): 39171-8.; J |
| | associated protein | | | binding | Biol Chem. 2002 Dec. 27; 277(52): 50860-6.; J |
| | 1(MRFAP1), T-cell | | | activity | Biol Chem. 2003 Dec. 5; 278(49): 49618-24.; Mol |
| | activation protein | | | | Cell Biol. 2004 October; 24(19): 8366-73. |
| | (PGR1) | | | | |
| FLJ39454 | WARP von Willebrand | NM_022834.3 | NP_073745.2 | | FEBS Lett. 2003 Sep. 25; 552(2-3): 91-4.; FEBS |
| | factor A domain- | NM_199121.1 | NP_954572.1 | | Lett. 2002 Apr. 24; 517(1-3): 61-6.; J Biol Chem. |
| | related protein | | | | 2006 Mar. 17; 281(11): 7341-9. |
| FLJ45115 | E1A binding protein | NM_015409.3 | NP_056224.2 | DNA binding activity, | Hum Genet. 1997 July; 100(1): 114-22.; J Biol |
| | p400 (EC 3.6.1.-) | | | RNA polymerase II | Chem. 2005 Jun. 10; 280(23): 21915-23.; DNA Res. |
| | (p400 kDa SWI2/SNF2- | | | transcription factor | 2000 Apr. 28; 7(2): 143-50.; DNA Res. 2001 Apr. |
| | related protein) | | | activity, enhancer | 27; 8(2): 85-95.; Genome Res. 2002 November; |
| | (Domino homolog) | | | binding activity, | 12(11): 1773-84.; Cell. 2001 Aug. 10; 106(3): |
| | (hDomino) (CAG | | | helicase activity, DNA | 297-307.; Genes Dev. 2005 Jan. 15; 19(2): |
| | repeat protein 32) | | | dependent transcription | 196-201.; EMBO J. 2006 Apr. 19; 25(8): 1680-9. |
| | (Trinucleotide repeat- | | | control activity, | |
| | containing gene 12 | | | immune response, | |
| | protein). | | | chromatin modification | |
| FLJ90066 | Cell cycle exit and | NM_016564.3 | NP_057648.2 | | Cell. 2006 May 19; 125(4): 801-14.; J Neurochem. |
| | neuronal | | | | 2005 October; 5(1): 146-59.; Biochem J. 2001 May |
| | differentiation | | | | 1; 355(Pt 3): 715-24.; Genome Res. 2006 |
| | protein 1; | | | | January; 16(1): 55-65. |
| | BM88 antigen. | | | | |
| FLJ37995 | Carbonic anhydrase | NM_198584.1 | NP_940986.1 | hydrocarbonation | J Biol Chem. 2004 Jan. 23; 279(4): 2719-27.; |
| | XIII (EC 42.1.1) | | | enzyme activity, | BMC Cancer. 2005 Apr. 18; 5(1): 41. |
| | (Carbonate | | | Zn ion binding | |
| | dehydratase XIII) | | | activity, lyase | |
| | (CA-XIII). | | | activity, one- | |
| | | | | carbon compound | |
| | | | | metabolism control | |

TABLE 2-18

| | | | | | |
|---|---|---|---|---|---|
| FLJ26058 | Elongation | NM_001404.4 | NP_001395.1 | translation | Genome Res. 2004 July; 14(7): 1324-32.; Nature. |
| | factor | | | elongation | 2005 Oct. 20; 437(7062): 1173-8.; Mol Cell. 2004 Sep. |
| | 1-gamma (EF-1- | | | factor activity, | 24; 15(6): 853-65.; Mol Cell Biochem. 1999 January; |
| | gamma) (eEF-1 | | | translation | 191(1-2): 181-6.; Nucleic Acids Res. 2000 Aug. |
| | B gamma) | | | elongation | 1; 28(15): 2866-72; Nucleic Acids Res. 1992 Nov. |
| | (PRO1608). | | | control, protein | 25; 20(22): 5907-10.; Protein Sci. 1994 |
| | | | | biosynthesis | November; 3(11): 2045-54.; Pancreas. 1992; 7(2): |
| | | | | control, | 144-52.; Proc Natl Acad Sci USA. 2001 Aug. |
| | | | | eucaryote | 28; 98(18): 10374-9.; Nucleic Acids Res. 1992 May |
| | | | | translation | 25; 20(10): 2598.; Biochem Biophys Res Commun. |
| | | | | elongation | 2002 Feb. 15; 291(1): 158-64.; J Biol Chem. 2003 |
| | | | | factor complex | Sep. 12; 278(37): 35325-36.; Curr Biol. 2004 Aug. |
| | | | | formation | 24; 14(16): 1436-50.; J Biol Chem. 1997 Dec. |
| | | | | | 26; 272(52): 33290-7. |
| FLJ46369 | Similar to c66 | none | none | | none |
| | SLIT-like | | | | |
| | testicular | | | | |
| | protein | | | | |
| | (FLJ43944 | | | | |
| | protein)(cDNA | | | | |
| | FLJ46369) | | | | |
| FLJ16517 | LIN28B, lin-28 | NM_001004317.2 | NP_001004317.1 | DNA binding | none |
| | homolog B | | | activity, DNA | |
| | (C. elegans) | | | dependent | |
| | | | | transcription | |
| | | | | control activity | |

TABLE 2-19

| | | | | | |
|---|---|---|---|---|---|
| FLJ26591 | Peptidyl-prolyl | NM_021130.3 | NP_066953.1 | peptidyl- | Nature. 2005 Oct. 20; 437(7062): 1173-8.; |
| | cis-trans | | | prolyl cis- | Biochemistry. 2006 Apr. 11; 45(14): 4664-73.; J Biol |
| | isomerase A | | | transisomerase | Chem. 2006 Jan. 13; 281(2): 1241-50.; Proteins. 2004 |
| | (PPlase) | | | activity, | Aug. 15; 56(3): 449-63.; J Virol. 2006 March; 80(6): 2855- |
| | (Rotamase) | | | cyclosporine | 62.; J Biol Chem. 2005 Jun. 24; 280(25): 23668-74.; |
| | (Cyclophilin A) | | | A binding | J Virol. 2005 January; 79(1): 176-83.; J Surg Res. 2005 |
| | (Cyclosporin A- | | | activity, binding | February; 123(2): 312-9.; J Infect Dis. 2005 Mar. |
| | binding protein). | | | activity to | 1; 191(5): 755-60.; Mol Ther. 2006 October; 14(4): 546-54.; |
| | | | | unfolded protein, | Immunol Lett. 2004 September; 95(2): 155-9.; J Cancer |
| | | | | protein folding | Res Clin Oncol. 2006 July; 132(7): 473-81.; Nat |
| | | | | control activity, | Methods. 2005 January; 2(1): 47-53.; Biochem Biophys |
| | | | | virion binding | Res Commun. 2004 Aug. 27; 321(3): 557-65.; Biochemistry. |
| | | | | activity,virus | 2004 Aug. 24; 43(33): 10605-18.; Mol Cancer Res. 2006 |
| | | | | genome replication | August; 4(8): 529-38.; J Proteome Res. 2005 May-June; |
| | | | | control function | 4(3): 931-40.; J Biol Chem. 2005 Jun. 10; 280(23): |
| | | | | | 21965-71.; Diabetologia. 2005 December; 48(12): 2576-81. |
| FLJ26596 | Histone H2B type | NM_003520.3 | NP_003511.1 | DNA binding | Nature. 2005 Oct. 20; 437(7062): 1173-8.; Virology. |
| | 1-N; Histone | | | activity, | 2000 Nov. 25; 277(2): 278-95.; Virology. 2001 Oct. |
| | H2B.d (H2B/d). | | | nucleosome | 25; 289(2): 312-26.; EMBO J. 2003 Dec. |
| | | | | association | 15; 22(24): 6550-61.; Genomics. 2002 November; 80(5): |
| | | | | control, | 487-98.; Biol Chem. 1999 January; 380(1): 7-18.; Hum |
| | | | | chromosome | Genet. 1997 December; 101(3): 284-94.; Mol Cell Biol. |
| | | | | organization and | 1998 May; 18(5): 2535-44. |
| | | | | biosynthesis | |
| FLJ90480 | Zinc finger CCCH- | NM_032527.2 | NP_115916.2 | nucleic acid | DNA Res. 2001 Apr. 27; 8(2): 85-95. |
| | type with G patch | NM_181484.1 | NP_852149.1 | binding activity | |
| | domain protein. | NM_181485.1 | NP_852150.1 | | |

TABLE 2-20

| | | | | | |
|---|---|---|---|---|---|
| FLJ43067 | Phosphoglycerate | NM_002629.2 | NP_002620.1 | diphosphoglycerate mutase | Ann Genet. 1982; 25(1): 25-7.; |
| | mutase 1 (EC 5.4.2.1) | | | activity, diphosphoglycerate | Acta Crystallogr D Biol |
| | (EC 5.4.2.4) | | | phosphatase activity, | Crystallogr. 2004 October; |
| | (EC 3.1.3.13) | | | hydrolase activity, | 60(Pt 10): 1893-4.; J Biol Chem. |
| | (Phosphoglycerate | | | isomerase activity, | 1988 Nov. 15; 263(32): 16899- |
| | mutase isozyme B) | | | phosphotransferase activity, | 905.; J Biol Chem. 1987 Oct. 25; |
| | (PGAM-B) (BPG- | | | glycolytic system control | 262(30): 14612-7.; Haematologica |
| | dependent PGAM 1). | | | | 2005 February; 90(2): 257-9.; J Biol |
| | | | | | Chem. 1988 Nov. 15; 263(32): |
| | | | | | 16906-10. |
| FLJ25460 | novel (Similar to | NM_138813.2 | NP_620168.1 | | Physiol Genomics. 1999 Nov. 11; |
| | other ORF of Potential | | | | 1(3): 139-50.; Biochim Biophys |
| | phospholipid- | | | | Acta. 2003 Jul. 21; 1633(2): 127- |
| | transporting ATPase IK | | | | 31.; Lab Anim. 1978 January; |
| | (ATPase class I type | | | | 12(1): 1-4. |
| | 8B member 3)gene) | | | | |
| FLJ26806 | RNA-binding region | XM_940318.2 | XP_945411.2 | | none |
| | RNP-1 (RNA | | | | |
| | recognition motif) | | | | |
| | domain containing | | | | |
| | protein(FLJ40411 | | | | |
| | protein) | | | | |
| FLJ43911 | C20 orf133: | NM_080676.5 | NP_542407.2 | | Genome Res. 2006 January; 16(1): |
| | chromosome 20 | NM_001033087.1 | NP_001028259.1 | | 55-65. |
| | open reading | | | | |
| | frame 133; Similar | | | | |
| | to Appr-1-p | | | | |
| | processing enzyme | | | | |
| | domain protein | | | | |
| FLJ44715 | FLJ44715 gene | none | none | | none |
| | product | | | | |
| FLJ90031 | Polymerase I and | NM_012232.2 | NP_036364.2 | RNA polymerase I | EMBO J. 1998 May 15; 17(10): |
| | transcript release | | | transcription end factor | 2855-64.; Biochem J. 2000 Apr. 1; |
| | factor(PTRF protein) | | | activity, RNA binding | 347 Pt 1: 55-9.; Biochem J. |
| | (FKSG13 protein) | | | activity, protein binding | 2004 Oct. 15; 383(Pt 2): 237-48. |
| | | | | activity, rRNA binding | |
| | | | | activity, rRNA primary | |
| | | | | transcription product binding | |
| | | | | activity, DNA dependent | |
| | | | | transcription control, | |
| | | | | transcription end control, | |
| | | | | transcription open control | |
| | | | | from RNA polymerase I | |
| | | | | promoter | |

As used herein, “a homologous protein” means a protein belonging to the same protein family as the above-described protein. Example homologous proteins are given in Tables 2-1 to 2-20.

As used herein, “a variant” of a protein means an artificial mutant or natural mutant of the protein, and includes splicing variants.

A variant of a protein provided by the present invention can also be, for example, a protein that consists of an amino acid sequence resulting from the substitution, deletion, addition or insertion of one or more amino acids in the amino acid sequence shown by SEQ ID NOs:1 to 63, and that interacts with a bioactive substance.

The number of amino acids substituted, deleted, added or inserted can be any one that allows the retention of the function of the protein to be provided in the present invention, for example, about 1 to 50, preferably about 1 to 30, more preferably about 1 to 20, further more preferably about 1 to 10, most preferably 1 to 5 or 1 or 2. The site for substitution, deletion, addition or insertion of an amino acid can be any site that allows the retention of the function, for example, a site other than functionally important domains.

Furthermore, a variant of a protein provided by the present invention can be a protein which consists of, for example, an amino acid sequence having a homology of about 50% or more, preferably about 70% or more, more preferably about 80% or more, further more preferably about 90% or more, most preferably about 95% or more (but excluding 100% homology), to the amino acid sequence shown by SEQ ID NOs:1 to 63, and which interacts with a bioactive substance. Here, the numerical values of the above-described homology are calculated by, for example, executing the commands for the maximum matching method using the DNASIS sequence analytical software (Hitachi Software Engineering). The parameters for the calculation should be used in default settings (initial settings).

When a target protein of the present invention is used, the protein may be a labeled supply or a non-labeled supply, or a mixture of a labeled supply protein and a non-labeled supply protein mixed in a specified ratio. Examples of the labeling substance include fluorescent substances such as FITC and FAM, luminescent substances such as luminol, luciferin and lucigenin, radioisotopes such as ³H, ¹⁴C, ³²P, ³⁵S, and ¹²³I, affinity substances such as biotin and streptavidin, and the like.

The target genes of the present invention may be any ones that encode the target proteins of the present invention. For example, the target genes of the present invention can be those corresponding to proteins comprising the above-described amino acid sequences. For example, proteins comprising the above-described amino acid sequences can be those corresponding to cDNA clones having nucleotide sequences corresponding to the FLJ nucleotide sequence accession numbers shown in Tables 1-1 to 1-8.

In the H-Invitational Database (H-InvDB), for example, cDNA clones that share a gene region on the human genome are classified as a cluster; the cDNA clones corresponding to the proteins of the present invention are given respective gene loci, namely, H-Inv locus IDs (and H-Inv cDNA IDs) shown in Tables 1-1 to 1-8. Hence, the target genes of the present invention can be cDNAs of the FLJ nucleotide sequence accession numbers shown in Tables 1-1 to 1-8, a cDNA cluster of H-Inv cDNA IDs in H-InvDB, or genes given H-Inv locus IDs or genes homologous thereto. As used herein, the target genes of the present invention are not limited to human genes, but include orthologues of different animal species.

As used herein, “a homologous gene” means a gene belonging to the same family of genes as the above-described genes. Examples of homologous genes are the genes that encode the homologous proteins shown in Tables 2-1 to 2-20.

As used herein, “a variant” of a gene means an artificial variant or natural variant of the gene, and includes splicing variants transcribed from the gene.

For example, a variant of a gene provided by the present invention can be a cDNA that consists of a nucleotide sequence that hybridizes to a sequence complementary to the nucleotide sequence corresponding to one of the FLJ nucleotide sequence accession numbers shown in Tables 1-1 to 1-8 under stringent conditions, and that corresponds to a protein that interacts with a bioactive substance. Here, “hybridize under stringent conditions” means that a positive hybridization signal remains observable even under conditions of, for example, heating in a solution of 6×SSC, 0.5% SDS and 50% formamide at 42° C., followed by washing in a solution of 0.1×SSC and 0.5% SDS at 68° C.

The target proteins and target genes of the present invention can be used for the development of drugs for diseases or conditions associated with bioactive substance X, or diseases or conditions associated with target gene Y (or target protein Y), or for the development of investigational reagents for the diseases or conditions, and the like. Diseases or conditions associated with bioactive substance X and diseases or conditions associated with target gene Y are described in detail below. (Diseases or conditions associated with bioactive substance X)

“A disease or condition associated with bioactive substance X” means a disease for which bioactive substance X is used or a disease corresponding to an adverse effect of bioactive substance X, or a condition for which use of bioactive substance X is desired (e.g., a deficiency of bioactive substance X) or an unwanted condition caused by bioactive substance X (e.g., an unwanted condition caused by excess intake of bioactive substance X). A disease or condition associated with bioactive substance X can be ameliorated or exacerbated by bioactive substance X.

“An action associated with a bioactive substance X” means an action of the same kind as, or opposite kind to, a kind of action actually exhibited by bioactive substance X (including pharmacological actions and adverse effects). In other words, an action associated with a bioactive substance X is an action capable of ameliorate or exacerbate “a disease or condition associated with bioactive substance X”. Hence, when the bioactive substance X is acetohexamide, the “action associated with a bioactive substance X” shows an insulin secretagogue action or a hypoglycemic effect and the like in pancreatic cells.

“A disease or condition associated with bioactive substance X” and “an action associated with a bioactive substance X” vary depending on the kind of bioactive substance X. Described below are “diseases or conditions associated with bioactive substance X” with reference to substances that represent bioactive substance X. Because “an action associated with a bioactive substance X” is any action capable of ameliorating or exacerbating “a disease or condition associated with bioactive substance X”, the following description of “diseases or conditions associated with bioactive substance X” will surely lead to the clarification of “actions associated with bioactive substance X”.

The disease relating to trimethylcolchicine acid means a disease to which trimethylcolchicine acid is applied or a disease corresponding to the side effect of trimethylcolchicine acid. Trimethylcolchicine acid is known as a therapeutic drug for gout (cell division inhibitor colchicine) analog. The disease to which trimethylcolchicine acid is applied is exemplified by gout and the like. On the other hand, the side effect of trimethylcolchicine acid is exemplified by gastrointestinal disorder (diarrhea, vomiting, abdominal pain) and the like. The action relating to trimethylcolchicine acid may be closely related to a target protein (target gene) thereof, for example, a protein containing the amino acid sequence shown by SEQ ID NO: 1 or SEQ ID NO: 2 or a homologous protein thereof or variants of them.

The disease relating to acenocoumarol means a disease to which acenocoumarol is applied or a disease corresponding to the side effect of acenocoumarol. Acenocoumarol is known as an antithrombotic agent (anticoagulant). The disease to which acenocoumarol is applied is exemplified by thromboembolism and the like. On the other hand, the side effect of acenocoumarol is exemplified by, bleeding (intraorgan bleeding such as cerebral hemorrhage, mucous membrane bleeding, subcutaneous hemorrhage and the like), skin necrosis (transient hypercoagulable state caused by sudden decrease in protein C activity), liver dysfunction·jaundice and the like. The action relating to acenocoumarol may be closely related to a target protein (target gene) thereof, for example, a protein containing the amino acid sequence shown by SEQ ID NO: 27 or a homologous protein thereof or variants of them.

The disease relating to paracetamol means a disease to which paracetamol is applied or a disease corresponding to the side effect of paracetamol. Paracetamol is known as an antipyretic·analgesic·anti-inflammatory agent (non-pyrazolone).

The disease to which paracetamol is applied is exemplified by headache, symptomatic neuralgia, low back pain, muscular pain, pain of a bruise, pain of sprain, menstrual cramps, postpartum pain, cancer pain, toothache, pain after dental treatment and the like. On the other hand, the side effect of paracetamol is exemplified by shock, anaphylactoid symptoms, mucocutaneous ocular syndrome, toxic epidermal necrosis, induction of asthma attack, liver dysfunction and the like. The action relating to paracetamol may be closely related to a target protein (target gene) thereof, for example, a protein containing the amino acid sequence shown by SEQ ID NO: 3 or a homologous protein thereof or variants of them.

The disease relating to acetohexamide means a disease to which acetohexamide is applied or a disease corresponding to the side effect of acetohexamide. Acetohexamide is known as a sulfonylurea-type oral hypoglycemic agent. The disease to which acetohexamide is applied is exemplified by insulin-nondependent type diabetes and the like. On the other hand, the side effect of acetohexamide is exemplified by hypoglycemia, feeling of weakness, extreme hunger, sweating, palpitation, tremor, headache, paresthesia, anxiety, excitation, nervousness, loss of concentration, mental disorder, consciousness disorder, twitch, aplastic anemia, hemolytic anemia, agranulocytosis and the like. The action relating to acetohexamide may be closely related to a target protein (target gene) thereof, for example, a protein containing the amino acid sequence shown by SEQ ID NO: 23, SEQ ID NO: 24 or SEQ ID NO: 34 or a homologous protein thereof or variants of them.

The disease relating to acetopromazine means a disease to which acetopromazine is applied or a disease corresponding to the side effect of acetopromazine. Acetopromazine is known as an antianxiety drug. The disease to which acetopromazine is applied is exemplified by schizophrenia, senile psychosis, manic psychosis, depression, sedative and hypnotic effect caused by nervous disease and the like. The action relating to acetopromazine may be closely related to a target protein (target gene) thereof, for example, a protein containing the amino acid sequence shown by SEQ ID NO: 36 or a homologous protein thereof or variants of them.

The disease relating to actinomycin D means a disease to which actinomycin D is applied or a disease corresponding to the side effect of actinomycin D. Actinomycin D is known as an anti-cancer agent, antibacterial substance (anti Gram-positive bacterium), DNA intercalator (RNA synthesis inhibitor). The disease to which actinomycin D is applied is exemplified by Wilms' tumor, chorioepithelioma, destructive hydatid mole and the like. On the other hand, the side effect of actinomycin D is exemplified by anorexia, nausea·vomiting, stomatitis, leucopenia, thrombocytopenia, hair loss, pigment deposition, generalized fatigability, nervousness, bone marrow suppress (aplastic anemia, agranulocytosis, pancytopenia), anaphylactoid reaction, dyspnea, hepatic vein obstruction, serious hepatopathy (with hepatomegaly, ascites and the like) and the like. The action relating to actinomycin D may be closely related to a target protein (target gene) thereof, for example, a protein containing the amino acid sequence shown by SEQ ID NO: 54 or a homologous protein thereof or variants of them.

The disease relating to ajmaline means a disease to which ajmaline is applied or a disease corresponding to the side effect of ajmaline. Ajmaline is known as an antiarrhythmic agent (Class I Na channel suppress). The disease to which ajmaline is applied is exemplified by extrasystole (supraventricular, ventricular), prophylaxis of paroxysmal tachycardia (supraventricular, ventricular), fresh atrial fibrillation, prophylaxis of paroxysmal atrial fibrillation, combination with electric shock therapy and maintain of sinus rate thereafter, and the like. On the other hand, the side effect of ajmaline is exemplified by agranulocytosis, jaundice, bundle branch block, anorexia, nausea·vomiting, diarrhea, headache, top-heavy feeling, dizziness, heat sensation, sense of numbness, sleepiness, palpitation and the like. The action relating to ajmaline may be closely related to a target protein (target gene) thereof, for example, a protein containing the amino acid sequence shown by SEQ ID NO: 1 or SEQ ID NO: 2 or a homologous protein thereof or variants of them.

The disease relating to albendazole means a disease to which albendazole is applied or a disease corresponding to the side effect of albendazole. Albendazole is known as an agent for parasite·protozoa (Echinococcus repellent). The disease to which albendazole is applied is exemplified by echinococcosis and the like. On the other hand, the side effect of albendazole is exemplified by liver·bile duct disorder (liver dysfunction), pancytopenia and the like. The action relating to albendazole may be closely related to a target protein (target gene) thereof, for example, a protein containing the amino acid sequence shown by SEQ ID NO: 38 or a homologous protein thereof or variants of them.

The disease relating to alfuzosin means a disease to which alfuzosin is applied or a disease corresponding to the side effect of alfuzosin. Alfuzosin is known as a depressor, a therapeutic drug for benign prostatic hyperplasia (BPH). The disease to which alfuzosin is applied is exemplified by benign prostatic hyperplasia (BPH) and the like. On the other hand, the side effect of alfuzosin is exemplified by dizziness·sleepiness, headache, abdominal pain, constipation, dyspepsia, nausea, impotence, bronchitis, pharyngitis, sinusitis and the like. The action relating to alfuzosin may be closely related to a target protein (target gene) thereof, for example, a protein containing the amino acid sequence shown by SEQ ID NO: 35 or a homologous protein thereof or variants of them.

The disease relating to α-methyl-5-hydroxytryptamine means a disease to which α-methyl-5-hydroxytryptamine is applied or a disease corresponding to the side effect of α-methyl-5-hydroxytryptamine. α-Methyl-5-hydroxytryptamine is known as a serotonin analog. The action of α-methyl-5-hydroxytryptamine is exemplified by 5-HT2 agonitic action (5-hydroxytryptamine 2A/2Creceptor agonist) and the like. The action relating to α-methyl-5-hydroxytryptamine may be closely related to a target protein (target gene) thereof, for example, a protein containing the amino acid sequence shown by SEQ ID NO: 30 or a homologous protein thereof or variants of them.

The disease relating to amoxapine means a disease to which amoxapine is applied or a disease corresponding to the side effect of amoxapine. Amoxapine is known as an antidepressant·a mood-stabilizing drug·a psychostimulant drug (monoamine re-uptake inhibitor). The disease to which amoxapine is applied is exemplified by depression·state of depression and the like. The side effect of amoxapine is exemplified by dysautonomia such as dry mouth·constipation and the like, dizziness·sleepiness, malignant syndrome, twitch·delirium tremens·hallucination·deliria, agranulocytosis, paralytic ileus (intestine paralysis), tardive dyskinesia and the like. The action relating to amoxapine may be closely related to a target protein (target gene) thereof, for example, a protein containing the amino acid sequence shown by SEQ ID NO: 36 or a homologous protein thereof or variants of them.

The disease relating to antipyrine means a disease to which antipyrine is applied or a disease corresponding to the to side effect of antipyrine. Antipyrine is known as a an antipyretic·analgesic·anti-inflammatory agent. The disease to which antipyrine is applied is exemplified by headache and the like. On the other hand, the side effect of antipyrine is exemplified by shock (precordial anxiety, lowering of blood pressure·facial pallor·pulse abnormalities·dyspnea etc.), agranulocytosis, anaphylaxis (rash·erythema, vesicular keratitis, itching etc.), thrombocytopenia, anemia and the like. The action relating to antipyrine may be closely related to a target protein (target gene) thereof, for example, a protein containing the amino acid sequence shown by SEQ ID NO: 1 or a homologous protein thereof or variants of them.

The disease relating to azithromycin means a disease to which azithromycin is applied or a disease corresponding to the side effect of azithromycin. Azithromycin is known as a macrolide antibiotic. The disease to which azithromycin is applied is exemplified by pharyngolaryngitis (throat abscess)·acute and chronic bronchitis·infectious bronchiectasis·secondary infection during chronic respiratory diseases·adenoiditis (periamygdalitis·peritonsillar abscess)·pneumonia·lung suppuration, tympanitis (including mastoiditis and petrositis), furuncle·anthracia·erysipelas·cellulitis·inflammation of a lymphatic vessel (lymph node)·whitlow·perionychia, urethritis, cervicitis, sinusitis, inflammation of periodontal tissue, pericoronitis, jaw inflammation and the like. On the other hand, the side effect of azithromycin is exemplified by diarrhea·loose stool, vomiting, urticarial eruption, eosinophilia, leucopenia, shock anaphylactoid symptoms (dyspnea, wheezing, angioedema etc.), skin mucocutaneous ocular syndrome, toxic epidermal necrosis, toxic epidermal necrosis, liver dysfunction·jaundice, severe colitis accompanying hematochezia such as pseudomembranous colitis and the like, interstitial pneumonia·eosinophilic pneumonia, QT prolonged·ventricular tachycardia and the like. The action relating to azithromycin may be closely related to a target protein (target gene) thereof, for example, a protein containing the amino acid sequence shown by SEQ ID NO: 62 or a homologous protein thereof or variants of them.

The disease relating to benzbromarone means a disease to which benzbromaroneis applied or a disease corresponding to the side effect of benzbromarone. Benzbromarone is known as a therapeutic drug for gout·hyperuricemia. The disease to which benzbromaroneis applied is exemplified by improvement of hyperuricemia in hypertension accompanying gout·hyperuricemia, and the like. In addition, the action of benzbromarone is exemplified by uric acid excretion promotion action and the like. On the other hand, the side effect of benzbromarone is exemplified by severe hepatopathy such as fulminant hepatitis and the like, jaundice, gastric distress, digestive trouble, itching sensation, rash, diarrhea and the like. The action relating to benzbromarone may be closely related to a target protein (target gene) thereof, for example, a protein containing the amino acid sequence shown by SEQ ID NO: 42, SEQ ID NO: 53 or SEQ ID NO: 54 or a homologous protein thereof or variants of them.

The disease relating to benzethonium means a disease to which benzethonium is applied or a disease corresponding to the side effect of benzethonium. Benzethonium is known as a sterilizing agent. The disease to which benzethonium is applied is exemplified by pharyngitis, adenoiditis, stomatitis, acute gingivitis, glossitis, wound of mouth cavity, and the like. On the other hand, the side effect of benzethonium is exemplified by rash, pruritus, irritating sensation of mouth cavity and pharynx, roughness in one's mouth, and the like. The action relating to benzethonium may be closely related to a target protein (target gene) thereof, for example, a protein containing the amino acid sequence shown by SEQ ID NO: 23, SEQ ID NO: 53 or SEQ ID NO: 61 or a homologous protein thereof or variants of them.

The disease relating to benzydamine means a disease to which benzydamine is applied or a disease corresponding to the side effect of benzydamine. Benzydamine is known as a topical non-steroidal antipyretic·analgesic·anti-inflammatory agent and gargle. The disease to which benzydamine is applied is exemplified by sore throat, dysphagia and the like, and the action of benzydamine is exemplified by antiphlogistic analgetic action, topical anesthetic action and the like. The action relating to benzydamine may be closely related to a target protein (target gene) thereof, for example, a protein containing the amino acid sequence shown by SEQ ID NO: 60 or a homologous protein thereof or variants of them.

The disease relating to berberine means a disease to which berberine is applied or a disease corresponding to the side effect of berberine. Berberine is known as a antidiarrheal drug·a drug for intestinal regulation. The disease to which berberine is applied is exemplified by diarrhea and the like. The action relating to berberine may be closely related to a target protein (target gene) thereof, for example, a protein containing the amino acid sequence shown by SEQ ID NO: 32 or a homologous protein thereof or variants of them.

The disease relating to bezafibrate means a disease to which bezafibrate is applied or a disease corresponding to the side effect of bezafibrate. Bezafibrate is known as a fibrate-type therapeutic drug for hyperlipidemia. The disease to which bezafibrate is applied is exemplified by hyperlipidemia and the like. On the other hand, the side effect of bezafibrate is exemplified by rhabdomyolysis, liver dysfunction, jaundice and the like. The action relating to bezafibrate may be closely related to a target protein (target gene) thereof, for example, a protein containing the amino acid sequence shown by SEQ ID NO: 39 or a homologous protein thereof or variants of them.

The disease relating to bicartamide means a disease to which bicartamide is applied or a disease corresponding to the side effect of bicartamide. Bicartamide is known as an anti-cancer agent (prostate cancer therapeutic agent). The disease to which bicartamide is applied is exemplified by prostate cancer and the like. On the other hand, the side effect of bicartamide is exemplified by liver dysfunction, jaundice, leucopenia, thrombocytopenia, interstitial pneumonia and the like. The action relating to bicartamide may be closely related to a target protein (target gene) thereof, for example, a protein containing the amino acid sequence shown by SEQ ID NO: 53 or a homologous protein thereof or variants of them.

The disease relating to boldine means a disease to which boldine is applied or a disease corresponding to the side effect of boldine. Boldine is known as an alkaloid contained in boldo leaf. The action of boldine is exemplified by antioxidant action, bilesecretagogue action, gastrointestinal function improving effect and the like. The action relating to boldine may be closely related to a target protein (target gene) thereof, for example, a protein containing the amino acid sequence shown by SEQ ID NO: 1 or a homologous protein thereof or variants of them.

The disease relating to bromperidol means a disease to which bromperidol is applied or a disease corresponding to the side effect of bromperidol. Bromperidol is known as a butyrophenone antipsychotic agent. The disease to which bromperidol is applied is exemplified by schizophrenia and the like. On the other hand, the side effect of bromperidol is exemplified by malignant syndrome (akinetic mutism, highly muscle stiffness, difficulty in swallowing, tachysystole, sweating etc.), tardive dyskinesia(involuntary movement around the mouth, involuntary movement of the limbs etc.), syndrome of inappropriate secretion of anti-diuretic hormone(SIADH), the intestine paralysis (anorexia, nausea·vomiting, remarkable constipation, swelling or laxity of the abdomen and enterostasis etc.), rhabdomyolysis and the like. The action relating to bromperidol may be closely related to a target protein (target gene) thereof, for example, a protein containing the amino acid sequence shown by SEQ ID NO: 33 or a homologous protein thereof or variants of them.

The disease relating to budesonide means a disease to which budesonide is applied or a disease corresponding to the side effect of budesonide. Budesonide is known as a adrenal corticosteroid, dermatological preparation or a therapeutic drug for bronchial asthma (dry powder type inhaled steroid). The disease to which budesonide is applied is exemplified by bronchial asthma and the like. On the other hand, the side effect of budesonide is exemplified by sore throat, hoarseness, nausea, cough and the like. The action relating to budesonide may be closely related to a target protein (target gene) thereof, for example, a protein containing the amino acid sequence shown by SEQ ID NO: 27 or a homologous protein thereof or variants of them.

The disease relating to bupivacaine means a disease to which bupivacaine is applied or a disease corresponding to the side effect of bupivacaine. Bupivacaine is known as a long-acting topical anesthetic. The action of bupivacaine is exemplified by epidural·conduction anesthetic action, intrathecal (spinal) anesthetic action and the like. On the other hand, the side effect of bupivacaine is exemplified by shock (bradycardia, arrhythmia, lowering of blood pressure, respiratory depression, cyanosis, disturbance of consciousness etc.), tremor, twitch, hepatopathy, abnormal sensation, perception·motion impairment and the like. The action relating to bupivacaine may be closely related to a target protein (target gene) thereof, for example, a protein containing the amino acid sequence shown by SEQ ID NO: 14 or a homologous protein thereof or variants of them.

The disease relating to buspirone means a disease to which buspirone is applied or a disease corresponding to the side effect of buspirone. Buspirone is known as an antianxiety drug. The disease to which buspirone is applied is exemplified by generalized anxiety disorder and the like. On the other hand, the side effect of buspironeis exemplified by dizziness, headache and the like. The action relating to buspirone may be closely related to a target protein (target gene) thereof, for example, a protein containing the amino acid sequence shown by SEQ ID NO: 29 or a homologous protein thereof or variants of them.

The disease relating to cefazolin means a disease to which cefazolin is applied or a disease corresponding to the side effect of cefazolin. Cefazolin is known as a cephem antibiotic. The disease to which cefazolin is applied is exemplified by cephalosporin antibiotic, infections with staphylococcus, streptococcus, pneumococcus, Escherichia coli, pneumobacillus and myxomycete (sepsis, subacute bacterial endocarditis, superficial suppurative disease group, deep suppurative disease group, respiratory infection, lung suppuration, empyema, pleurisy, biliary infection, peritonitis, urinary tract infection, gynecological infections, otological infections) and the like. On the other hand, the side effect of cefazolin is exemplified by shock, anaphylactoid symptoms, blood disorder (pancytopenia, agranulocytosis), hepatopathy (jaundice and the like), renopathy (acute renal failure and the like), colitis (pseudomembranous colitis and the like), skin disorder (skin mucocutaneous ocular syndrome, toxic epidermal necrosis), interstitial pneumonia, PIE syndrome, twitch and the like. The action relating to cefazolin may be closely related to a target protein (target gene) thereof, for example, a protein containing the amino acid sequence shown by SEQ ID NO: 59 or a homologous protein thereof or variants of them.

The disease relating to celestine blue means a disease to which celestine blue is applied or a disease corresponding to the side effect of celestine blue. Celestine blue is known as a cell stain used to stain cell nucleus·chromosome and the like. The action relating to celestine blue may be closely related to a target protein (target gene) thereof, for example, a protein containing the amino acid sequence shown by SEQ ID NO:1, SEQ ID NO: 2, SEQ ID NO:3, SEQ ID NO: 32 or SEQ ID NO: 46 or a homologous protein thereof or variants of them.

The disease relating to cephaeline means a disease to which cephaeline is applied or a disease corresponding to the side effect of cephaeline. Cephaeline is known as an ipecac alkaloid. The disease to which cephaeline is applied is exemplified by emetic action (stomach mucous membrane stimuli action) and the like. The action relating to cephaeline may be closely related to a target protein (target gene) thereof, for example, a protein containing the amino acid sequence shown by SEQ ID NO: 1 or SEQ ID NO: 36 or a homologous protein thereof or variants of them.

The disease relating to chlordiazepoxide means a disease to which chlordiazepoxide is applied or a disease corresponding to the side effect of chlordiazepoxide. Chlordiazepoxide is known as a sedative hypnotic and benzodiazepine antianxiety agent. The disease to which chlordiazepoxide is applied is exemplified by anxiety·tension·depression which are caused by neurosis, anxiety·tension which are caused by depression, physical symptom caused by psychosomatic disorder (stomach·duodenal ulcer, hypertension) and anxiety˜tension·depression and the like. On the other hand, the side effect of chlordiazepoxide is exemplified by abstinence symptom such as drug dependence, convulsive attack, deliria, tremor, insomnia, anxiety, hallucination, delusion and the like, stimulus and excitement·confusion and the like which are caused by schizophrenia and the like, respiratory depression caused by respiratory diseases such as chronic bronchitis and the like, and the like. The action relating to chlordiazepoxide may be closely related to a target protein (target gene) thereof, for example, a protein containing the amino acid sequence shown by SEQ ID NO: 56 or a homologous protein thereof or variants of them.

The disease relating to chlorogenic acid means a disease to which chlorogenic acid is applied or a disease corresponding to the side effect of chlorogenic acid. Chlorogenic acid is known as a kind of polyphenol contained a lot in coffee and tomato. The action of chlorogenic acid is exemplified by antioxidant action, central nervous excitatory action and the like. The action relating to chlorogenic acid may be closely related to a target protein (target gene) thereof, for example, a protein containing the amino acid sequence shown by SEQ ID NO: 27 or a homologous protein thereof or variants of them.

The disease relating to chlorothiazide means a disease to which chlorothiazide is applied or a disease corresponding to the side effect of chlorothiazide. Chlorothiazide is known as a diuretic. The disease to which chlorothiazide is applied is exemplified by essential hypertension and the like. On the other hand, the side effect of chlorothiazide is exemplified by hypokalemia, hyponatremia, hypochloraemic alkalosis, hyperuricemia and the like. The action relating to chlorothiazide may be closely related to a target protein (target gene) thereof, for example, a protein containing the amino acid sequence shown by SEQ ID NO: 27 or a homologous protein thereof or variants of them.

The disease relating to chromomycin A3 means a disease to which chromomycin A3 is applied or a disease corresponding to the side effect of chromomycin A3. Chromomycin A3 is known as an anti-cancer agent. The disease to which chromomycin A3 is applied is exemplified by various tumor and the like. The action relating to chromomycin A3 may be closely related to a target protein (target gene) thereof, for example, a protein containing the amino acid sequence shown by SEQ ID NO: 17 or SEQ ID NO: 34 or a homologous protein thereof or variants of them.

The disease relating to ciclopirox means a disease to which ciclopiroxis applied or a disease corresponding to the side effect of ciclopirox. Ciclopirox is known as an antifungal agent for skin. The disease to which ciclopirox is applied is exemplified by ringworm (ringworm of body, ringworm of crotch, trichophytia pompholyciformis), candidiasis (intertrigo, erythema blastomyceticum infantile, erosio interdigitalis) and the like. On the other hand, the side effect of ciclopirox is exemplified by dermatitis, skin stimuli action and the like. The action relating to ciclopiroxmay be closely related to a target protein (target gene) thereof, for example, a protein containing the amino acid sequence shown by SEQ ID NO: 1 or SEQ ID NO: 3 or a homologous protein thereof or variants of them.

The disease relating to cisapride means a disease to which cisapride is applied or a disease corresponding to the side effect of cisapride. Cisapride is known as a gastrointestinal drug (gastric motility activation-regulation agent). The disease to which cisapride is applied is exemplified by erosive esophagitis and the like. On the other hand, the side effect of cisapride is exemplified by QT prolonged, ventricular arrhythmia and the like. The action relating to cisapride may be closely related to a target protein (target gene) thereof, for example, a protein containing the amino acid sequence shown by SEQ ID NO: 31 or a homologous protein thereof or variants of them.

The disease relating to clarithromycin means a disease to which clarithromycin is applied or a disease corresponding to the side effect of clarithromycin. Clarithromycin is known as a macrolide antibiotic. The disease to which clarithromycin is applied is exemplified by general infections (staphylococcus, streptococcus, peptostreptococcus, haemophilus influenzae, bordetella pertussis, campylobacter, mycoplasma, chlamydia):folliculitis, furunculosis, anthracia, erysipelas, cellulitis, lymphangitis, whitlow, perionychia, subcutaneous abscess, hidradenitis, chronic pyoderma, perianal abscess, superficial secondary infection of trauma·burn·operative wound and the like, pharyngol aryngitis, acute bronchitis, adenoiditis, chronic bronchitis, diffuse panbronchiolitis, bronchiectasis (during infection), secondary infection of chronic respiratory diseases, pneumonia, lung suppuration, nongonococcal urethritis, campylobacter enteritis, cervicitis, tympanitis, sinusitis, inflammation of periodontal tissue, pericoronitis, jaw inflammation, pharyngolaryngitis, malignant scarlet fever, pertussis, disseminated mycobacterial infection accompanied by acquired immunodeficiency syndrome (AIDS), Helicobacter pylori infection in gastric ulcer or duodenal ulcer, and the like. On the other hand, the side effect of clarithromycin is exemplified by shock, anaphylactoid symptoms, QT prolonged, ventricular tachycardia, fulminant hepatitis, liver dysfunction, jaundice, liver failure, thrombocytopenia, pancytopenia, hemolytic anemia, leucopenia, agranulocytosis, skin mucocutaneous ocular syndrome, toxic epidermal necrosis, PIE syndrome·interstitial pneumonia, pseudomembranous colitis, hemorrhagic colitis, rhabdomyolysis, twitch, allergic purpura, acute renal failure and the like. The action relating to clarithromycin may be closely related to a target protein (target gene) thereof, for example, a protein containing the amino acid sequence shown by SEQ ID NO: 49 or a homologous protein thereof or variants of them.

The disease relating to clemizole means a disease to which clemizole is applied or a disease corresponding to the side effect of clemizole. Clemizole is known as a topical anesthetics. The disease to which clemizole is applied is exemplified by itching accompanied by dermatic diseases (eczema·dermatitis, drug eruption, intoxication dermatosis, strophulus infantum, bite and stab wound), hives, hay fever, remission of symptom of hemorrhoid·anal fissure·mild proctitis, and the like. On the other hand, the side effect of clemizole is exemplified by topical fungus·virus·bacterium infectious diseases, skin irritating sensation, itching sensation and the like. The action relating to clemizole may be closely related to a target protein (target gene) thereof, for example, a protein containing the amino acid sequence shown by SEQ ID NO: 22 or SEQ ID NO: 47 or a homologous protein thereof or variants of them.

The disease relating to clenbuterol means a disease to which clenbuterol is applied or a disease corresponding to the side effect of clenbuterol. Clenbuterol is a β2-stimulant and is known as a therapeutic agent for stress urinary incontinence, broncho dilator·a drug for asthma. The disease to which clenbuterol is applied is exemplified by remission of various symptom such as dyspnea and the like based on airway obstructive disorder such as bronchial asthma·chronic bronchitis·emphysema·acute bronchitis, stress urinary incontinence and the like. On the other hand, the side effect of clenbuterol is exemplified by tremor, abdominal pain, elevation of blood pressure, severe decreased serum potassium value and the like. The action relating to clenbuterol may be closely related to a target protein (target gene) thereof, for example, a protein containing the amino acid sequence shown by SEQ ID NO: 23, SEQ ID NO: 36 or SEQ ID NO: 60 or a homologous protein thereof or variants of them.

The disease relating to clobetasone means a disease to which clobetasone is applied or a disease corresponding to the side effect of clobetasone. Clobetasone is an adrenal corticosteroid and is known as an antiphlogistic·analgesic·antipruritic agent (dermatological preparation). The disease to which clobetasone is applied is exemplified by atopic dermatitis (including infantile eczema), facial·neck·axillary·genital eczema and dermatitis, and the like. On the other hand, the side effect of clobetasone is exemplified by hypertonia oculi·glaucoma·posterior subcapsular cataract which are caused by application to eyelid skin, skin infections, steroid acne, peristome dermatitis, steroid cutaneous, hypersensitivity, suppression of pituitary gland·adrenal cortical function, and the like. The action relating to clobetasone may be closely related to a target protein (target gene) thereof, for example, a protein containing the amino acid sequence shown by SEQ ID NO: 35 or a homologous protein thereof or variants of them.

The disease relating to clofazimine means a disease to which clofazimine is applied or a disease corresponding to the side effect of clofazimine. Clofazimine is known as a therapeutic drug for Hansen's disease. The disease to which clofazimine is applied is exemplified by Hansen's disease (multibacillary, erythema nodosum leprosum) and the like. On the other hand, the side effect of clofazimine is exemplified by chromatosis, low vision, enterostasis, splenic infarction, embolized thrombus and the like. The action relating to Clofazimine may be closely related to a target protein (target gene) thereof, for example, a protein containing the amino acid sequence shown by SEQ ID NO: 15, SEQ ID NO: 37, SEQ ID NO: 53 or SEQ ID NO: 54 or a homologous protein thereof or variants of them.

The disease relating to clofilium means a disease to which clofilium is applied or a disease corresponding to the side effect of clofilium. Clofilium is a K channel blocker and is known as an antiarrhythmic agent·cardiac depression agent. The disease to which clofilium is applied is exemplified by arrhythmia and the like. The action relating to clofilium may be closely related to a target protein (target gene) thereof, for example, a protein containing the amino acid sequence shown by SEQ ID NO: 1 or a homologous protein thereof or variants of them.

The disease relating to clomiphene means a disease to which clomiphene is applied or a disease corresponding to the side effect of clomiphene. Clomiphene is known as an ovulation inducing agent. The disease to which clomiphene is applied is exemplified by induction of ovulation in infertility based on ovulation disorder, male infertility and the like. On the other hand, the side effect of clomiphene is exemplified by ovarian enlargement caused by ovary hyperstimulation, vision disorder, nausea, vomiting, headache and the like. The action relating to clomiphene may be closely related to a target protein (target gene) thereof, for example, a protein containing the amino acid sequence shown by SEQ ID NO: 23 or a homologous protein thereof or variants of them.

The disease relating to clopamide means a disease to which clopamide is applied or a disease corresponding to the side effect of clopamide. Clopamide is known as a thiazide diuretic and depressor. The disease to which clopamide is applied is exemplified by hypertension, edema and the like. On the other hand, the side effect of clopamide is exemplified by nausea, vomiting, headache, feebleness, convulsion, low blood pressure, misty vision and the like. The action relating to clopamide may be closely related to a target protein (target gene) thereof, for example, a protein containing the amino acid sequence shown by SEQ ID NO: 23 or a homologous protein thereof or variants of them.

The disease relating to colchicine means a disease to which colchicine is applied or a disease corresponding to the side effect of colchicine. Colchicine is known as a therapeutic drug for gout·hyperuricemia. The disease to which colchicine is applied is exemplified by remission and prophylaxis of gouty attack, and the like. On the other hand, the side effect of colchicine is exemplified by aplastic anemia, granulocyte decrease, leucopenia, thrombocytopenia, rhabdomyolysis, myopathy, peripheral nerve disorders and the like. The action relating to colchicine may be closely related to a target protein (target gene) thereof, for example, a protein containing the amino acid sequence shown by SEQ ID NO: 59 or a homologous protein thereof or variants of them.

The disease relating to colistin means a disease to which colistin is applied or a disease corresponding to the side effect of colistin. Colistin is known as a antibiotic. The disease to which colistin is applied is exemplified by enteritis (colitis)·dysenteria and the like caused by colistin-sensitive strain of Escherichia coli·dysenteria. On the other hand, the side effect of colistin is exemplified by anaphylaxis (rash, itching sensation etc.), nausea·vomiting, anorexia, diarrhea etc. and the like. The action relating to colistin may be closely related to a target protein (target gene) thereof, for example, a protein containing the amino acid sequence shown by SEQ ID NO: 62 or a homologous protein thereof or variants of them.

The disease relating to conessine means a disease to which conessine is applied or a disease corresponding to the side effect of conessine. Conessine is a steroid alkaloid and is known as an antidiarrheic and antibiotic. The disease to which conessine is applied is exemplified by amebic dysentery, vaginal trichomoniasis and the like. The action relating to conessine may be closely related to a target protein (target gene) thereof, for example, a protein containing the amino acid sequence shown by SEQ ID NO: 1 or a homologous protein thereof or variants of them.

The disease relating to coniine (DL) means a disease to which coniine (DL) is applied or a disease corresponding to the side effect of coniine (DL). Coniine (DL) is a very toxic component of Conium maculatum and is known as a pseudo alkaloid. The action of coniine (DL) is exemplified by muscle relaxant action, and the disease to which coniine (DL) is applied is exemplified by spasmolysis, fever and the like. On the other hand, the side effect of coniine (DL) is exemplified by sleepiness, vomiting, respiratory depression and the like. The action relating to coniine (DL) may be closely related to a target protein (target gene) thereof, for example, a protein containing the amino acid sequence shown by SEQ ID NO: 1 or SEQ ID NO: 3 or a homologous protein thereof or variants of them.

The disease relating to coralyne means a disease to which coralyne is applied or a disease corresponding to the side effect of coralyne. Coralyne is known as a berberine alkaloid. The action of coralyne is exemplified by antitumor action and the like. The action relating to coralyne may be closely related to a target protein (target gene) thereof, for example, a protein containing the amino acid sequence shown by SEQ ID NO: 33 or a homologous protein thereof or variants of them.

The disease relating to cyclobenzaprinepurine means a disease to which cyclobenzaprinepurine is applied'or a disease corresponding to the side effect of cyclobenzaprinepurine. Cyclobenzaprinepurine is known as a central muscle relaxant. The disease to which cyclobenzaprinepurine is applied is exemplified by twitch and the like. On the other hand, the side effect of cyclobenzaprinepurine is exemplified by sleepiness, weakness, hallucination and the like. The action relating to cyclobenzaprinepurine may be closely related to a target protein (target gene) thereof, for example, a protein containing the amino acid sequence shown by SEQ ID NO: 23 or a homologous protein thereof or variants of them.

The disease relating to cyclopentolate means a disease to which cyclopentolate is applied or a disease corresponding to the side effect of cyclopentolate. Cyclopentolate is known as a mydriatic. The disease to which cyclopentolate is applied is exemplified by accommodation paralysis (ophthalmology) and the like. The action relating to cyclopentolate may be closely related to a target protein (target gene) thereof, for example, a protein containing the amino acid sequence shown by SEQ ID NO: 36 or a homologous protein thereof or variants of them.

The disease relating to cyclosporine A means a disease to which cyclosporine A is applied or a disease corresponding to the side effect of cyclosporine A. Cyclosporine A is known as an immunosuppressant. The disease to which cyclosporine A is applied is exemplified by rejection suppress at kidney·liver·heart transplantation, suppress of rejection at bone marrow transplantation and graft-versus-host disease, Behcet's disease with eye symptom, psoriasis vulgaris, pustular psoriasis, psoriatic erythroderma, arthropathic psoriasis, aplastic anemia, pure red cell anemia, nephrosissyndrome and the like. On the other hand, the side effect of cyclosporineA is exemplified by shock (injection), renopathy, hepatopathy, central nervous system disorder, neuro-Behcet's disease symptom, infections, acute pancreatitis, thrombosis microvascular damage, hemolytic anemia, thrombocytopenia, rhabdomyolysis, lymphoma, lymphoproliferative disease, malignant tumor (particularly skin), elevation of blood pressure, anemia, leucopenia, thrombocytopenia, peptic ulcer, nausea, vomiting, abdominal pain, gastric distress, hypertrichiasis, tremor, headache, numbness, dizziness, glucosuria, hyperglycemia, hyperkalemia, hyperuricemia and the like. The action relating to cyclosporine A may be closely related to a target protein (target gene) thereof, for example, a protein containing the amino acid sequence shown by SEQ ID NO: 50 or a homologous protein thereof or variants of them.

The disease relating to diclofenac means a disease to which diclofenac is applied or a disease corresponding to the side effect of diclofenac. Diclofenac is known as a non-steroidal antipyretic·analgesic·anti-inflammatory agent. The disease to which diclofenac is applied is exemplified by analgesia and anti-inflammation in chronic rheumatoid arthritis·osteoarthritis·spondylitis deformans·lumbago·periarthritis humeroscapularis·peritendinitis·neck-shoulder-arm syndrome·muscular pain (muscular·fascial lumbago etc.)·neuralgia·afterpains·pelvic inflammation·dysmenorrhea·cystitis·anterior eye inflammation, posttraumatic tumentia·pain, prevention of inflammatory conditions after cataract surgery, and the like. On the other hand, the side effect of diclofenac is exemplified by shock, anaphylactoid symptoms, gastrointestinal ulceration with hemorrhagic shock or perforations, aplastic anemia, hemolytic anemia, agranulocytosis, thrombocytopenia, skin mucocutaneous ocular syndrome, toxic epidermal necrosis, erythroderma (exfoliative dermatitis), acute renal failure (interstitial nephritis, renal papillary necrosis etc.), severe asthmatic attack, interstitial pneumonia, congestive heart failure, sterile meningitis, severe hepatopathy, acute encephalopathy, rhabdomyolysis, diffuse superficial keratitis, corneal erosion, corneal ulcer, cornea perforations and the like. The action relating to diclofenac may be closely related to a target protein (target gene) thereof, for example, a protein containing the amino acid sequence shown by SEQ ID NO: 27 or a homologous protein thereof or variants of them.

The disease relating to diclofenamide means a disease to which diclofenamide is applied or a disease corresponding to the side effect of diclofenamide. Diclofenamide is known as a therapeutic drug for glaucoma. The disease to which diclofenamide is applied is exemplified by glaucoma and the like. On the other hand, the side effect of diclofenamide is exemplified by perception abnormality, anorexia, feebleness, sleepiness, headache, vomiting, dry mouth, depression, electrolyte imbalance (hypokalemia etc.), loss of muscle strength, constipation, confusion, dizziness and the like. The action relating to diclofenamide may be closely related to a target protein (target gene) thereof, for example, a protein containing the amino acid sequence shown by SEQ ID NO: 51 or a homologous protein thereof or variants of them.

The disease relating to diflunisal means a disease to which diflunisal is applied or a disease corresponding to the side effect of diflunisal. Diflunisal is known as an antipyretic·analgesic·anti-inflammatory agent. The disease to which diflunisal is applied is exemplified by an antipyretic·analgesic·anti-inflammatory agent, headache, symptomatic neuralgia, lumbago, muscular pain, pain of a bruise, pain of a sprain, menorrhalgia, postpartum pain, cancer pain, toothache, pain after dental treatment, and the like. On the other hand, the side effect of diflunisal is exemplified by peptic ulcer, gastrointestinal haemorrhagia, gastrointestinal perforations, gastric distress, abdominal pain, nausea, diarrhea, stomatitis, dry mouth, vomiting, anorexia, dyspepsia, gastritis, abdominal distension, constipation, sleepiness, insomnia, dizziness, headache, sweating, depression, nervousness, perception abnormality, rash, urticaria, itching, redness, jaundice, acute interstitial nephritis, thrombocytopenia, eosinophilia, edema, feebleness and the like. The action relating to diflunisal may be closely related to a target protein (target gene) thereof, for example, a protein containing the amino acid sequence shown by SEQ ID NO: 32 or a homologous protein thereof or variants of them.

The disease relating to dihydrostreptomycin means a disease to which dihydrostreptomycin is applied or a disease corresponding to the side effect of dihydrostreptomycin. Dihydrostreptomycin is known as a antibiotic (mainly, animal drug). The disease to which dihydrostreptomycin is applied is exemplified by bacterium infections and the like. The action relating to dihydrostreptomycin may be closely related to a target protein (target gene) thereof, for example, a protein containing the amino acid sequence shown by SEQ ID NO: 19 or a homologous protein thereof or variants of them.

The disease relating to diperodon means a disease to which diperodon is applied or a disease corresponding to the side effect of diperodon. Diperodon is known as a topical anesthetics (skin agent). The disease to which diperodon is applied is exemplified by topical (skin) anesthesia for excoriation·irritation·pruritus, elimination of discomfort caused by hemorrhoid (intrarectal administration) and the like. The action relating to diperodon may be closely related to a target protein (target gene) thereof, for example, a protein containing the amino acid sequence shown by SEQ ID NO: 27 or a homologous protein thereof or variants of them.

The disease relating to difenidol means a disease to which difenidol is applied or a disease corresponding to the side effect of difenidol. Difenidol is known as a vestibular nucleus blocker. The disease to which difenidol is applied is exemplified by dizziness and the like. On the other hand, the side effect of difenidol is exemplified by dizziness, unstable feeling, hallucination, headache, confusion, ocular accommodation disorder, mydriasis, dry mouth, anorexia, abdomen uncomfortable feeling, nausea·vomiting, palpitation, facial heat sensation, dysuria and the like. The action relating to difenidol may be closely related to a target protein (target gene) thereof, for example, a protein containing the amino acid sequence shown by SEQ ID NO: 1 or a homologous protein thereof or variants of them.

The disease relating to dipyridamole means a disease to which dipyridamole is applied or a disease corresponding to the side effect of dipyridamole. Dipyridamole is known as a antianginal drug (colonary vasodilator). The disease to which dipyridamole is applied is exemplified by angina pectoris, myocardial infarction (excluding acute phase), other ischemic cardiac diseases, congestive heart failure, supression of thrombus·embolus after cardiac valve replacement surgery in combination with warfarin, decrease of urine protein in chronic glomerulonephritis·nephrosis syndrome which are resistant to steroid, and the like. On the other hand, the side effect of dipyridamole is exemplified by progression of angina pectoris symptom, hemorrhagic diathesis, thrombocytopenia, anaphylaxis such as bronchial spasm·angioedema and the like, and the like. The action relating to dipyridamole may be closely related to a target protein (target gene) thereof, for example, a protein containing the amino acid sequence shown by SEQ ID NO: 15 or a homologous protein thereof or variants of them.

The disease relating to dizocilpine means a disease to which dizocilpine is applied or a disease corresponding to the side effect of dizocilpine. Dizocilpine is known as a noncompetitive and selective NMDA receptor antagonist. The action of dizocilpine is exemplified by antidepressive action, antiischemic action, neuroprotective action in retinal ganglion cell disorder, and the like. The action relating to dizocilpine may be closely related to a target protein (target gene) thereof, for example, a protein containing the amino acid sequence shown by SEQ ID NO: 25 or a homologous protein thereof or variants of them.

The disease relating to DO897/99 means a disease to which DO897/99 is applied or a disease corresponding to the side effect of DO897/99. DO897/99 is known as a dopamine receptor antagonists. The action of DO897/99 is exemplified by dopamine receptor antagonistic action and the like. The action relating to DO897/99 may be closely related to a target protein (target gene) thereof, for example, a protein containing the amino acid sequence shown by SEQ ID NO: 27 or SEQ ID NO: 34 or a homologous protein thereof or variants of them.

The disease relating to domperidone means a disease to which domperidone is applied or a disease corresponding to the side effect of domperidone. Domperidone is known as a gastrointestinal function promotility agent. The disease to which domperidone is applied is exemplified by disease such as chronic gastritis·gastroptosis·postgastrectomy syndrome·periodic vomiting·upper respiratory tract infection and the like, and mitigation of gastrointestinal symptoms (nausea, vomiting, anorexia, abdominal distension, abdominal pain, heartburn and the like) caused by administration of pharmaceutical agent (anti-malignant tumor agent or levodopa preparation), and the like. On the other hand, the side effect of domperidone is exemplified by diarrhea, defecation desire, abdominal pain, anaphylactoid symptoms, extrapyramidal symptom (Parkinsonian symptom) such as tremor·muscle rigidity and the like, liver dysfunction, gynecomastia, increase of prolactin, milk secretion, distention of the breast, menstrual disorder, palpitation, sweating, sleepiness, dizziness and the like. The action relating to Domperidone may be closely related to a target protein (target gene) thereof, for example, a protein containing the amino acid sequence shown by SEQ ID NO: 34, SEQ ID NO: 36, SEQ ID NO: 53 or SEQ ID NO: 54 or a homologous protein thereof or variants of them.

The disease relating to dopamine means a disease to which dopamine is applied or a disease corresponding to the side effect of dopamine. Dopamine is a catecholamine and is known as a cardiac stimulants. The disease to which dopamine is applied is exemplified by acute circulatory failure (cardiogenic shock·hemorrhagic shock), acute circulatory failure condition and the like. On the other hand, the side effect of dopamine is exemplified by arrhythmia, tachysystole, vomiting, paralytic ileus, peripheral ischemia·gangrene such as cold sense of limb and the like caused by peripheral vasoconstriction, and the like. The action relating to dopamine may be closely related to a target protein (target gene) thereof, for example, a protein containing the amino acid sequence shown by SEQ ID NO: 30 or a homologous protein thereof or variants of them.

The disease relating to doxazosin means a disease to which doxazosin is applied or a disease corresponding to the side effect of doxazosin. Doxazosin is known as a antiadrenergic (a blockers). The disease to which doxazosin is applied is exemplified by hypertension, hypertension caused by melanocytoma, benign prostatic hyperplasia (BPH) and the like. On the other hand, the side effect of doxazosin is exemplified by faint·unconsciousness, orthostatic hypotension, arrhythmia, cerebrovascular disorder, angina pectoris, myocardial infarction, agranulocytosis, leucopenia, thrombocytopenia, liver dysfunction and the like. The action relating to doxazosin may be closely related to a target protein (target gene) thereof, for example, a protein containing the amino acid sequence shown by SEQ ID NO: 1, SEQ ID NO: 35, SEQ ID NO: 53 or SEQ ID NO: 61 or a homologous protein thereof or variants of them.

The disease relating to doxycycline means a disease to which doxycycline is applied or a disease corresponding to the side effect of doxycycline. Doxycycline is known as a tetracycline antibiotic. The disease to which doxycycline is applied is exemplified by superficial suppurative disease (adenoiditis, pharyngitis, abscess, whitlow, folliculitis, dacryocystitis, wound and burn infection, postoperative infection) caused by staphylococcus, streptococcus, pneumococcus, gonococcus, pneumobacillus, Escherichia coli, dysenteria, deep suppurative disease (mastitis, lymphadenitis, myelitis), bronchitis, bronchial pneumonia, pneumonia, bronchiectasis, dysenteria, cholangitis, cholecystitis, urinary tract infection (pyelitis, pyelonephritis, cystitis, urethritis), prostatitis, uterine adnexitis, intrauterine infection, gonorrhea, malignant scarlet fever, conjunctivitis, keratitis, corneal ulcer, tympanitis, sinusitis, sialadenitis and the like. On the other hand, the side effect of doxycycline is exemplified by shock, anaphylactoid symptoms (dyspnea, blood vessel neurotic edema etc.), skin mucocutaneous ocular syndrome, toxic epidermal necrosis, exfoliative dermatitis, pseudomembranous colitis, hepatitis, liver dysfunction, jaundice and the like. The action relating to doxycycline may be closely related to a target protein (target gene) thereof, for example, a protein containing the amino acid sequence shown by SEQ ID NO: 59 or a homologous protein thereof or variants of them.

The disease relating to eburnamonine means a disease to which eburnamonine is applied or a disease corresponding to the side effect of eburnamonine. Eburnamonine is known as an alkaloid contained in an extract of vinca minor. The action of eburnamonine is exemplified by brain metabolism improving effect and the like. The possible disease wherein eburnamonine has a pharmacological action is exemplified by dementia, memory, concentration power, tinnitus, vision, improvement in neurological·psychological symptom such as blueness and the like, and the like. The action relating to eburnamonine may be closely related to a target protein (target gene) thereof, for example, a protein containing the amino acid sequence shown by SEQ ID NO: 10 or SEQ ID NO: 44 or a homologous protein thereof or variants of them.

The disease relating to etodolac means a disease to which etodolac is applied or a disease corresponding to the side effect of etodolac. Etodolac is known as a non-steroidal antipyretic·analgesic·anti-inflammatory agent. The disease to which etodolac is applied is exemplified by chronic rheumatoid arthritis·osteoarthritis·lumbago·periarthritis humeroscapularis·cervicobrachial syndrome·peritendinitis·anti-inflammation and analgesia after surgery and trauma, and the like. On the other hand, the side effect of etodolac is exemplified by shock, anaphylactoid symptoms, peptic ulcer, skin mucocutaneous ocular syndrome, pancytopenia, hemolytic anemia, agranulocytosis, thrombocytopenia, acute renal failure (interstitial nephritis, renal papillary necrosis etc.), acute aggravation in chronic renal failure, liver dysfunction, jaundice, congestive heart failure, eosinophilic pneumonia, interstitial pneumonia and the like. The action relating to etodolac may be closely related to a target protein (target gene) thereof, for example, a protein containing the amino acid sequence shown by SEQ ID NO: 23 or a homologous protein thereof or variants of them.

The disease relating to fenbendazole means a disease to which fenbendazole is applied or a disease corresponding to the side effect of fenbendazole. Fenbendazole is known as a drug for parasite˜protozoan (mainly animal drug). The action of fenbendazole is exemplified by parasiticidal action and the like. The action relating to fenbendazole may be closely related to a target protein (target gene) thereof, for example, a protein containing the amino acid sequence shown by SEQ ID NO: 22 or a homologous protein thereof or variants of them.

The disease relating to fenbufen means a disease to which fenbufen is applied or a disease corresponding to the side effect of fenbufen. Fenbufen is known as a prodrug of non-steroidal antipyretic·analgesic·anti-inflammatory agent. The disease to which fenbufen is applied is exemplified by rheumatoid arthritis, arthritis accompanied by collagen disease, gout attack, osteoarthritis, lumbago, periarthritis humeroscapularis, neck-shoulder-arm syndrome, anti-inflammation·analgesia·pyretolysis in cord·peritendinitis, remission of inflammation and swelling after trauma·surgery and extraction of a tooth, and the like. On the other hand, the side effect of fenbufen is exemplified by digestive symptom, peptic ulcer·gastrointestinal haemorrhagia, gastric pain·abdominal pain, anorexia, stomatitis, rash·urticarial eruption, melaena, hematemesis, severe skin symptom (high fever, rash·redness, sore of lip and intraoral sore, throat pain, interstitial pneumonia, induced asthmatic attack and the like. The action relating to fenbufen may be closely related to a target protein (target gene) thereof, for example, a protein containing the amino acid sequence shown by SEQ ID NO: 59 or a homologous protein thereof or variants of them.

The disease relating to fenoprofen means a disease to which fenoprofen is applied or a disease corresponding to the side effect of fenoprofen. Fenoprofen is known as a non-steroidal antipyretic·analgesic·anti-inflammatory agent. The disease to which fenoprofen is applied is exemplified by pyretolysis·analgesia in acute upper respiratory infection·acute bronchitis, chronic rheumatoid arthritis·osteoarthritis·lumbago·neck-shoulder-arm syndrome·periarthritis humeroscapularis·anti-inflammation·analgesia after trauma·surgery and extraction of a tooth, and the like. On the other hand, the side effect of fenoprofen is exemplified by gastric distress·gastric pain and the like digestive symptom, shock·anaphylactoid symptoms, skin mucocutaneous ocular syndrome, toxic epidermal necrosis, agranulocytosis, acute renal failure(interstitial nephritis, renal papillary necrosis etc.)·nephrosis syndrome, gastrointestinal tract perforations and the like. The action relating to fenoprofen may be closely related to a target protein (target gene) thereof, for example, a protein containing the amino acid sequence shown by SEQ ID NO: 26 or a homologous protein thereof or variants of them.

The disease relating to flumequine means a disease to which flumequine is applied or a disease corresponding to the side effect of flumequine. Flumequine is known as an antibacterial antibiotic. The action relating to flumequine may be closely related to a target protein (target gene) thereof, for example, a protein containing the amino acid sequence shown by SEQ ID NO: 56 or a homologous protein thereof or variants of them.

The disease relating to flupentixol means a disease to which flupentixol is applied or a disease corresponding to the side effect of flupentixol. Flupentixol is known as a antipsychotic agents. The action of flupentixol is exemplified by sedative action (psychomotor excitation, impulsivity suppress), anti-abnormal experience (improvement of hallucination·delusion and the like), activation effect (improvement of impaired mental activity) and the like. On the other hand, the side effect of flupentixol is exemplified by Parkinson's symptom, acute dystonia (eyeball supraduction, neck spastic torsion, tongue thrusting, difficulty in swallowing), akathisia, autonomic symptoms (dry mouth·sweating•constipation•orthostatic hypotension•reflex tachycardia•sleepiness), tardive dyskinesia and the like. The action relating to flupentixol may be closely related to a target protein (target gene) thereof, for example, a protein containing the amino acid sequence shown by SEQ ID NO: 23 or SEQ ID NO: 34 or a homologous protein thereof or variants of them.

The disease relating to fluphenazine means a disease to which fluphenazine is applied or a disease corresponding to the side effect of fluphenazine. Fluphenazine is known as a phenothiazine antipsychotic agent. The disease to which fluphenazine is applied is exemplified by schizophrenia and the like. On the other hand, the side effect of fluphenazine is exemplified by malignant syndrome, sudden death, aplastic anemia, hemolytic anemia, plateletanemia, paralytic ileus, tardive dyskinesia, SIADH, ophthalmopathy, SLE-like symptom, liver dysfunction, jaundice, irritationsymptom, optic hyperesthesia, leucopenia, agranulocytosis, thrombocytopenic purpura, hepatopathy, hypotensive, tachysystole, extrapyramidal symptom, miosis, confusion, insomnia and the like. The action relating to fluphenazine may be closely related to a target protein (target gene) thereof, for example, a protein containing the amino acid sequence shown by SEQ ID NO: 34 or SEQ ID NO: 61 or a homologous protein thereof or variants of them.

The disease relating to fluvoxamine means a disease to which fluvoxamine is applied or a disease corresponding to the side effect of fluvoxamine. Fluvoxamine is a selective serotonin reuptake inhibitor (SSRI) and is known as an antidepressant•mood-stabilizing drug•psychostimulant drug. The disease to which fluvoxamine is applied is exemplified by depression, state of depression, obsessive disorder and the like. On the other hand, the side effect of fluvoxamine is exemplified by digestion tract disorder (nausea, nausea, dry mouth, constipation), sleepiness, dizziness, twitch, shock, anaphylactoid symptoms, serotonin syndrome, malignant syndrome in combination with psychotropic drugs (antipsychotic agents•antidepressant etc.), leucopenia, thrombocytopenia, liver dysfunction, jaundice, hyponatremia, decreased plasma osmolality, increase of urinary sodium, hypersthenuria, syndrome of inappropriate secretion of anti-diuretic hormone (SIADH) accompanying with disturbance of consciousness and the like, and the like. The action relating to fluvoxamine may be closely related to a target protein (target gene) thereof, for example, a protein containing the amino acid sequence shown by SEQ ID NO: 25 or a homologous protein thereof or variants of them.

The disease relating to furazolidone means a disease to which furazolidone is applied or a disease corresponding to the side effect of furazolidone. Furazolidone is known as a synthesis antibacterial agent (mainly animal drug). The disease to which furazolidone is applied is exemplified by bacterial diarrhea caused by swine Salmonella•Escherichia coli, vibrio disease•furunculosis•Bacterial Gill Disease of fish and the like. On the other hand, the side effect of furazolidone is exemplified by carcinogenic possibility and the like. The action relating to furazolidone may be closely related to a target protein (target gene) thereof, for example, a protein containing the amino acid sequence shown by SEQ ID NO: 52 or a homologous protein thereof or variants of them.

The disease relating to gabapentin means a disease to which gabapentin is applied or a disease corresponding to the side effect of gabapentin. Gabapentin is known as an analgesic, a therapeutic drug for neuropathic pain (neuralgia) and an anti-convulsion drug. The disease to which gabapentin is applied is exemplified by various pain including neuropathic pain (neuralgia), post-herpes neuralgia, convulsion and the like. The action relating to gabapentin may be closely related to a target protein (target gene) thereof, for example, a protein containing the amino acid sequence shown by SEQ ID NO: 59 or a homologous protein thereof or variants of them.

The disease relating to GBR12909 means a disease to which GBR12909 is applied or a disease corresponding to the side effect of GBR12909. GBR12909 is known as a plasma membrane dopamine transporter inhibitor, thus, dopamine reuptake inhibitor. The disease to which GBR12909 is applied is exemplified by depression, cocaine addiction and the like. The action relating to GBR12909 may be closely related to a target protein (target gene) thereof, for example, a protein containing the amino acid sequence shown by SEQ ID NO: 61 or a homologous protein thereof or variants of them.

The disease relating to glibenclamide means a disease to which glibenclamide is applied or a disease corresponding to the side effect of glibenclamide. Glibenclamide is known as a sulfonylurea oral hypoglycemic drug. The disease to which glibenclamide is applied is exemplified by insulin-nondependent type diabetes and the like. On the other hand, the side effect of glibenclamide is exemplified by hypoglycemia, agranulocytosis, hemolytic anemia, hepatitis, liver dysfunction, jaundice and the like. The action relating to glibenclamide may be closely related to a target protein (target gene) thereof, for example, a protein containing the amino acid sequence shown by SEQ ID NO: 23 or SEQ ID NO: 37 or a homologous protein thereof or variants of them.

The disease relating to glipizide means a disease to which glipizide is applied or a disease corresponding to the side effect of glipizide. Glipizide is known as an oral hypoglycemic drug. The disease to which glipizide is applied is exemplified by insulin-nondependent type diabetes and the like. On the other hand, the side effect of glipizide is exemplified by hypoglycemia, agranulocytosis, hemolytic anemia, hepatitis, liver dysfunction, jaundice and the like. The action relating to glipizide may be closely related to a target protein (target gene) thereof, for example, a protein containing the amino acid sequence shown by SEQ ID NO: 23 or a homologous protein thereof or variants of them.

The disease relating to gramicidin means a disease to which gramicidin is applied or a disease corresponding to the side effect of gramicidin. Gramicidin is known as a antibiotic (peptide based, bacteriostasis action). The disease to which gramicidin is applied is exemplified by topical (for skin) peptide-based antibacterial agent, eczema•dermatitis with moistening•erosion•scab or secondary infection, psoriasis, palmoplantar pustulosis, burn and the like. On the other hand, the side effect of gramicidin is exemplified by skin infections (fungus disease, virus infections and the like), acne-like rash•rosacea-like dermatitis•peristome dermatitis caused by long-term consecutive use, cutaneous hypersensitivity, pituitary gland•adrenal cortex function suppression, hypertonia oculi•glaucoma caused by application to eyelid skin, and the like. The action relating to gramicidin may be closely related to a target protein (target gene) thereof, for example, a protein containing the amino acid sequence shown by SEQ ID NO: 53 or a homologous protein thereof or variants of them.

The disease relating to guanfacine means a disease to which guanfacine is applied or a disease corresponding to the side effect of guanfacine. Guanfacine is a sympathetic nerve suppressant (central α2 agonist) and is known as a depressor. The disease to which guanfacine is applied is exemplified by essential hypertension and the like. On the other hand, the side effect of guanfacine is exemplified by dry mouth, dizziness•lightheadedness, sleepiness, feebleness, headache, orthostatic hypotension, and the like. The action relating to guanfacine may be closely related to a target protein (target gene) thereof, for example, a protein containing the amino acid sequence shown by SEQ ID NO: 23 or a homologous protein thereof or variants of them.

The disease relating to harmol means a disease to which harmol is applied or a disease corresponding to the side effect of harmol. Harmol is known as an alkaloid contained in Passifloraceae plant. The possible action of harmol is exemplified by sedative action, anti-anxiety•tranquilization and the like. The action relating to harmol may be closely related to a target protein (target gene) thereof, for example, a protein containing the amino acid sequence shown by SEQ ID NO: 22 or a homologous protein thereof or variants of them.

The disease relating to hydroflumethiazide means a disease to which hydroflumethiazide is applied or a disease corresponding to the side effect of hydroflumethiazide. Hydroflumethiazide is known as a thiazide diuretic. The disease to which hydroflumethiazide is applied is exemplified by hypertension, congestive heart failure and the like. The action relating to hydroflumethiazide may be closely related to a target protein (target gene) thereof, for example, a protein containing the amino acid sequence shown by SEQ ID NO: 11 or a homologous protein thereof or variants of them.

The disease relating to hydroxychloroquine means a disease to which hydroxychloroquine is applied or a disease corresponding to the side effect of hydroxychloroquine. Hydroxychloroquine is known as an antimalarial drug and anti-rheumatic drug. The disease to which hydroxychloroquine is applied is exemplified by malaria, rheumatism and the like. The action relating to hydroxychloroquine may be closely related to a target protein (target gene) thereof, for example, a protein containing the amino acid sequence shown by SEQ ID NO: 52 or a homologous protein thereof or variants of them.

The disease relating to hydroxytacrine(R,S) means a disease to which hydroxytacrine(R,S) is applied or a disease corresponding to the side effect of hydroxytacrine(R,S). Hydroxytacrine(R,S) is known as a therapeutic drug for Alzheimer type dementia. The disease to which hydroxytacrine(R,S) is applied is exemplified by Parkinson's disease, Alzheimer type dementia and the like. The action relating to hydroxytacrine(R,S) may be closely related to a target protein (target gene) thereof, for example, a protein containing the amino acid sequence shown by SEQ ID NO: 43 or a homologous protein thereof or variants of them.

The disease relating to ifosfamide means a disease to which ifosfamide is applied or a disease corresponding to the side effect of ifosfamide. Ifosfamide is known as an anti-cancer agent (alkylating agent). The disease to which ifosfamide is applied is exemplified by small cell lung cancer, prostate cancer, cancer of the uterine cervix, osteosarcoma and the like. On the other hand, the side effect of ifosfamide is exemplified by bone marrow suppress, hemorrhagic cystitis, dysuria, Fanconi syndrome, disturbance of consciousness, encephalopathy, interstitial pneumonia, pneumonedema, cardiac muscle disorder, arrhythmia, syndrome of inappropriate secretion of anti-diuretic hormone(SIADH), acute pancreatitis and the like. The action relating to ifosfamide may be closely related to a target protein (target gene) thereof, for example, a protein containing the amino acid sequence shown by SEQ ID NO: 22 or a homologous protein thereof or variants of them.

The disease relating to iobenguane means a disease to which iobenguane is applied or a disease corresponding to the side effect of iobenguane. Iobenguane is known as an anti-cancer agent. The disease to which iobenguane is applied is exemplified by diagnosis of melanocytoma•neuroblastoma or medullary thyroid carcinoma using scintiography, and the like. The action relating to iobenguane may be closely related to a target protein (target gene) thereof, for example, a protein containing the amino acid sequence shown by SEQ ID NO: 9 or a homologous protein thereof or variants of them.

The disease relating to iproniazide means a disease to which iproniazide is applied or a disease corresponding to the side effect of iproniazide. Iproniazide is known as an antidepressant•mood-stabilizing drug•psychostimulant drug. The disease to which iproniazide is applied is exemplified by depression•state of depression and the like. On the other hand, the side effect of iproniazide is exemplified by hepatopathy, high blood pressure crisis (acute elevation of blood pressure) and the like. The action relating to iproniazide may be closely related to a target protein (target gene) thereof, for example, a protein containing the amino acid sequence shown by SEQ ID NO: 19 or a homologous protein thereof or variants of them.

The disease relating to isoxicam means a disease to which isoxicam is applied or a disease corresponding to the side effect of isoxicam. Isoxicam is known as an antipyretic•analgesic•anti-inflammatory agent. On the other hand, the side effect of isoxicam is exemplified by skin phototoxicity, toxic epidermal necrolysis, skin mucocutaneous ocular syndrome and the like. The action relating to isoxicam may be closely related to a target protein (target gene) thereof, for example, a protein containing the amino acid sequence shown by SEQ ID NO: 23 or a homologous protein thereof or variants of them.

The disease relating to isradipine means a disease to which isradipine is applied or a disease corresponding to the side effect of isradipine. Isradipine is known as a Ca antagonist. The disease to which isradipine is applied is exemplified by hypertension, Ca antagonist and the like. On the other hand, the side effect of isradipine is exemplified by headache, edema, dizziness, constipation, feebleness, face flush, abdomen uncomfortable feeling, rash and the like. The action relating to isradipine may be closely related to a target protein (target gene) thereof, for example, a protein containing the amino acid sequence shown by SEQ ID NO: 24 or a homologous protein thereof or variants of them.

The disease relating to josamycin means a disease to which josamycin is applied or a disease corresponding to the side effect of josamycin. Josamycin is known as a macrolide antibiotic. The disease to which josamycin is applied is exemplified by infections with staphylococcus, hemolysis streptococcus, pneumococcus, Haemophilus influenzae and micoplasma, pyoderma, impetigo, furuncle, anthracia, abscess, pharyngolaryngitis, adenoiditis, angina, acute upper respiratory infection, external otitis, gingivitis, eyelid inflammation, dacryocystitis, acute chronic bronchitis, pneumonia, bronchial pneumonia, primary atypical pneumonia, malignant scarlet fever, tympanitis, sinusitis, infections in dental region (periostitis, pericementitis, alveolitis, pericoronitis of wisdom tooth, arthritis, jaw inflammation, alveolar abscess, gingiva abscess) and the like. On the other hand, the side effect of josamycin is exemplified by diarrhea•loose stool, decreased appetite, nausea, vomiting, pseudomembranous colitis and the like. The action relating to josamycin may be closely related to a target protein (target gene) thereof, for example, a protein containing the amino acid sequence shown by SEQ ID NO: 49 or a homologous protein thereof or variants of them.

The disease relating to ketoprofen means a disease to which ketoprofen is applied or a disease corresponding to the side effect of ketoprofen. Ketoprofen is known as a non-steroidal antipyretic•analgesic•anti-inflammatory agent. The disease to which ketoprofen is applied is exemplified by chronic rheumatoid arthritis, osteoarthritis, lumbago, neck-shoulder-arm syndrome, symptomatic neuralgia, periarthritis humeroscapularis, herpes zoster, erythema exsudativum multiforme, erythema nodosum, acute upper respiratory infection, various cancers, gout attack, symptomatic neuralgia, muscular pain, analgesia•anti-inflammation•pyretolysis after trauma or surgery, and the like. On the other hand, the side effect of ketoprofen is exemplified by shock, anaphylactoid symptoms, peptic ulcer, gastrointestinal haemorrhagia such as hematemesis•melaena and the like, toxic epidermal necrosis, acute renal failure, nephrosis syndrome and the like. The action relating to ketoprofen may be closely related to a target protein (target gene) thereof, for example, a protein containing the amino acid sequence shown by SEQ ID NO: 59 or a homologous protein thereof or variants of them.

The disease relating to 3-hydroxykynurenine means a disease to which 3-hydroxykynurenine is applied or a disease corresponding to the side effect of 3-hydroxykynurenine. 3-Hydroxykynurenine is known to have epilepsy-like convulsion inductive action. The action relating to 3-hydroxykynurenine may be closely related to a target protein (target gene) thereof, for example, a protein containing the amino acid sequence shown by SEQ ID NO: 25 or a homologous protein thereof or variants of them.

The disease relating to leuprolide means a disease to which leuprolide is applied or a disease corresponding to the side effect of leuprolide. Leuprolide is known as a synthesis peptide analog of gonadotropin-releasing hormone. The disease to which leuprolide is applied is exemplified by endometriosis control, hypermenorrhea, reduction of myoma nucleus or improvement of symptom in myoma nucleus with lower abdominal pain•lumbago and anemia and the like, premenopausal breast cancer, prostate cancer, central precocious puberty and the like. On the other hand, the side effect of leuprolide is exemplified by interstitial pneumonia, anaphylactoid symptoms, liver dysfunction, jaundice, state of depression and the like. The action relating to leuprolide may be closely related to a target protein (target gene) thereof, for example, a protein containing the amino acid sequence shown by SEQ ID NO: 50 or a homologous protein thereof or variants of them.

The disease relating to L-thyroxine means a disease to which L-thyroxine is applied or a disease corresponding to the side effect of L-thyroxine. L-thyroxine is a thyroid gland hormone preparation and is known as a therapeutic drug for thyroid gland dysfunction. The disease to which L-thyroxine is applied is exemplified by cretinism, hypothyroidism (primary and hypophysial), mucoid edema, goiter and the like. On the other hand, the side effect of L-thyroxine is exemplified by angina pectoris, congestive heart failure and the like. The action relating to L-thyroxine may be closely related to a target protein (target gene) thereof, for example, a protein containing the amino acid sequence shown by SEQ ID NO: 34 or a homologous protein thereof or variants of them.

The disease relating to lidoflazine means a disease to which lidoflazine is applied or a disease corresponding to the side effect of lidoflazine. Lidoflazine is known as an antianginal drug. The disease to which lidoflazine is applied is exemplified by angina pectoris, arrhythmia and the like. The action relating to lidoflazine may be closely related to a target protein (target gene) thereof, for example, a protein containing the amino acid sequence shown by SEQ ID NO: 59 or a homologous protein thereof or variants of them.

The disease relating to α-lobeline (−) means a disease to which α-lobeline (−) is applied or a disease corresponding to the side effect of α-lobeline (−). α-Lobeline (−) is an alkaloid of Platycodon plant and are known as a ganglionic agonist (nicotinic partial agonist). The disease to which α-lobeline (−) is applied is exemplified by respiratory stimulus by chemoreceptor stimulation, quit smoking aid and the like. The action relating to α-lobeline (−) may be closely related to a target protein (target gene) thereof, for example, a protein containing the amino acid sequence shown by SEQ ID NO: 6 or a homologous protein thereof or variants of them.

The disease relating to loperamide means a disease to which loperamide is applied or a disease corresponding to the side effect of loperamide. Loperamide is known as an antidiarrheal drug•a drug for intestinal regulation. The disease to which loperamide is applied is exemplified by diarrhea, acute diarrhea and the like. On the other hand, the side effect of loperamide is exemplified by ileus-like symptom, anaphylactoid symptoms, rash, liver dysfunction, abdominal distension, nausea•vomiting, dry mouth, sleepiness, dizziness, sweating and the like. The action relating to loperamide may be closely related to a target protein (target gene) thereof, for example, a protein containing the amino acid sequence shown by SEQ ID NO: 15 or SEQ ID NO: 54 or a homologous protein thereof or variants of them.

The disease relating to maprotiline means a disease to which maprotiline is applied or a disease corresponding to the side effect of maprotiline. Maprotiline is known as an antidepressant•mood-stabilizing drug•psychostimulant drug (monoaminere uptake inhibitory). The disease to which maprotiline is applied is exemplified by depression•state of depression and the like. On the other hand, the side effect of maprotiline is exemplified by malignant syndrome, epilepsy attack, rhabdomyolysis, skin mucocutaneous ocular syndrome, agranulocytosis, paralytic ileus, interstitial pneumonia, eosinophilic pneumonia, QT prolonged, ventricular tachycardia, liver dysfunction, jaundice and the like. The action relating to maprotiline may be closely related to a target protein (target gene) thereof, for example, a protein containing the amino acid sequence shown by SEQ ID NO: 23 or SEQ ID NO: 63 or a homologous protein thereof or variants of them.

The disease relating to mebendazole means a disease to which mebendazole is applied or a disease corresponding to the side effect of mebendazole. Mebendazole is known as an agent for parasite•protozoa (agent destructive to whipworm). The disease to which mebendazole is applied is exemplified by trichuriasis and the like. On the other hand, the side effect of mebendazole is exemplified by hepatopathy, rash and the like in the long-term administration case. The action relating to mebendazole may be closely related to a target protein (target gene) thereof, for example, a protein containing the amino acid sequence shown by SEQ ID NO: 32 or a homologous protein thereof or variants of them.

The disease relating to meclofenamic acid means a disease to which meclofenamic acid is applied or a disease corresponding to the side effect of meclofenamic acid. Meclofenamic acid is known as an antipyretic•analgesic•anti-inflammatory agent (animal drug). The disease to which meclofenamic acid is applied is exemplified by chronic inflammatory disease, pelvic dysplasia•osteoarthritis and the like. On the other hand, the side effect of meclofenamic acid is exemplified by diarrhea, vomiting, digestion tract disorder and the like. The action relating to meclofenamic acid may be closely related to a target protein (target gene) thereof, for example, a protein containing the amino acid sequence shown by SEQ ID NO: 17 or a homologous protein thereof or variants of them.

The disease relating to metanephrine (D,L) means a disease to which metanephrine (D,L) is applied or a disease corresponding to the side effect of metanephrine (D,L). Metanephrine (D,L) is known as a cardiac stimulants. The action of metanephrine (D,L) is exemplified by cardiotonic action and the like. The action relating to metanephrine (D,L) may be closely related to a target protein (target gene) thereof, for example, a protein containing the amino acid sequence shown by SEQ ID NO: 52 or a homologous protein thereof or variants of them.

The disease relating to metaproterenol means a disease to which metaproterenol is applied or a disease corresponding to the side effect of metaproterenol. Metaproterenol is a β2-adrenoceptor stimulant and are known as a bronchodilator. The disease to which metaproterenol is applied is exemplified by asthma and the like. The action relating to metaproterenol may be closely related to a target protein (target gene) thereof, for example, a protein containing the amino acid sequence shown by SEQ ID NO: 43 or a homologous protein thereof or variants of them.

The disease relating to metergotamine means a disease to which metergotamine is applied or a disease corresponding to the side effect of metergotamine. Metergotamine is known as a 5-HT₂ antagonist. The action of metergotamine is exemplified by analgesic action in migraine, hypophysial and hypothalamic hormone action and the like. The action relating to metergotamine may be closely related to a target protein (target gene) thereof, for example, a protein containing the amino acid sequence shown by SEQ ID NO: 25 or SEQ ID NO: 43 or a homologous protein thereof or variants of them.

The disease relating to methimazole means a disease to which methimazole is applied or a disease corresponding to the side effect of methimazole. Methimazole is a hormone preparation and are known as a therapeutic drug for thyroid gland dysfunction (antithyroid agent). The disease to which methimazole is applied is exemplified by hyperthyroidism (Graves' disease, Basedow's disease) and the like. On the other hand, the side effect of methimazole is exemplified by agranulocytosis, eosinophilia, leucopenia, hemolytic anemia, thrombocytopenia and the like. The action relating to methimazole may be closely related to a target protein (target gene) thereof, for example, a protein containing the amino acid sequence shown by SEQ ID NO: 12 or a homologous protein thereof or variants of them.

The disease relating to methoxamine means a disease to which methoxamine is applied or a disease corresponding to the side effect of methoxamine. Methoxamine is known as a non-catecholamine vasopressor. The disease to which methoxamine is applied is exemplified by hypotensive state associated with anesthesia, paroxysmal supraventricular tachycardia and the like. The action relating to methoxamine may be closely related to a target protein (target gene) thereof, for example, a protein containing the amino acid sequence shown by SEQ ID NO: 25 or a homologous protein thereof or variants of them.

The disease relating to methoxy-6-harmalan means a disease to which methoxy-6-harmalan is applied or a disease corresponding to the side effect of methoxy-6-harmalan. Methoxy-6-harmalan is known as a narcotic. The action of methoxy-6-harmalan is exemplified by hallucinogenic action, antidepressive action and the like. The action relating to methoxy-6-harmalan may be closely related to a target protein (target gene) thereof, for example, a protein containing the amino acid sequence shown by SEQ ID NO: 1 or SEQ ID NO: 2 or a homologous protein thereof or variants of them.

The disease relating to mifepristone means a disease to which mifepristone is applied or a disease corresponding to the side effect of mifepristone. Mifepristone is known as an aborticide. The disease to which mifepristone is applied is exemplified by endometrial abortifacient and the like. On the other hand, the side effect of mifepristone is exemplified by nausea, vomiting, diarrhea, abdominal pain, headache, dizziness, feebleness, convulsion, haemorrhagia, vaginal secretion abnormality, vaginal uncomfortableness, fever, palpitation, faint, sepsis and the like. The action relating to mifepristone may be closely related to a target protein (target gene) thereof, for example, a protein containing the amino acid sequence shown by SEQ ID NO: 42 or a homologous protein thereof or variants of them.

The disease relating to minaprine means a disease to which minaprine is applied or a disease corresponding to the side effect of minaprine. Minaprine is known as an antidepressant, a cognitive enhancer, a brain circulation metabolism improving agent. The disease to which minaprine is applied is exemplified by antidepressant and cognitive enhancer and the like. The action relating to minaprine may be closely related to a target protein (target gene) thereof, for example, a protein containing the amino acid sequence shown by SEQ ID NO: 2 or a homologous protein thereof or variants of them.

The disease relating to minocycline means a disease to which minocycline is applied or a disease corresponding to the side effect of minocycline. Minocycline is known as a tetracycline antibiotic. The disease to which minocycline is applied is exemplified by following infections which are caused by staphylococcus•streptococcus•pneumococcus•Escherichia coli•citrobacter•klebsiella•enterobacter•chlamydiae•rickettsia, anthrax: sepsis, superficial suppurative disease (furuncle, impetigo, abscess, adenoiditis, pharyngolaryngitis, upper respiratory infection, dacryocystitis, stomatitis, pericementitis, periodontitis), deep suppurative disease (lymphadenitis, osteitis, inflammation around bone), bronchitis, pneumonia, parrot disease, malignant scarlet fever, tympanitis, sinusitis, parotitis, tsutsugamushi, anthrax and the like. On the other hand, the side effect of minocycline is exemplified by shock, anaphylactoid symptoms, aggravation of systemic lupus erythematosus (SLE)-like symptom, skin mucocutaneous ocular syndrome, toxic epidermal necrosis, blood disorder (pancytopenia, agranulocytosis, granulocyte decrease, leucopenia, thrombocytopenia, anemia), severe liver dysfunction (liver failure etc.), acute renal failure, interstitial nephritis, dyspnea, interstitial pneumonia, pancreatitis, psychoneurotic disorder (twitch, disturbance of consciousness etc.) and the like. The action relating to minocycline may be closely related to a target protein (target gene) thereof, for example, a protein containing the amino acid sequence shown by SEQ ID NO: 36 or a homologous protein thereof or variants of them.

The disease relating to misoprostol means a disease to which misoprostol is applied or a disease corresponding to the side effect of misoprostol. Misoprostol is a prostaglandin E1 derivative and are known as a therapeutic drug for peptic ulcera (mucus production•secretion promoting agent). The disease to which misoprostol is applied is exemplified by gastric ulcer and duodenal ulcer and the like caused by long-term administration of non-steroidal antiphlogistic analgetic. On the other hand, the side effect of misoprostol is exemplified by digestive symptom (diarrhea•loose stool, abdominal pain, abdominal distension, nausea, dyspepsia), shock,anaphylactoid symptoms and the like. The action relating to misoprostol may be closely related to a target protein (target gene) thereof, for example, a protein containing the amino acid sequence shown by SEQ ID NO: 23 or a homologous protein thereof or variants of them.

The disease relating to molsidomine means a disease to which molsidomine is applied or a disease corresponding to the side effect of molsidomine. Molsidomine is known as an antianginal drug. The disease to which molsidomine is applied is exemplified by angina pectoris and the like. The action relating to molsidomine may be closely related to a target protein (target gene) thereof, for example, a protein containing the amino acid sequence shown by SEQ ID NO: 4 or a homologous protein thereof or variants of them.

The disease relating to moroxydine means a disease to which moroxydine is applied or a disease corresponding to the side effect of moroxydine. Moroxydine is known as an antivirus agent. The disease to which moroxydine is applied is exemplified by herpes zoster, remission of various symptoms in upper respiratory tract infection caused by influenza•virus, pharyngoconjunctival fever caused by adenovirus, and the like. The action relating to moroxydine may be closely related to a target protein (target gene) thereof, for example, a protein containing the amino acid sequence shown by SEQ ID NO: 7 or a homologous protein thereof or variants of them.

The disease relating to moxalactam means a disease to which moxalactam is applied or a disease corresponding to the side effect of moxalactam. Moxalactam is known as a cephem antibiotic. The disease to which moxalactam is applied is exemplified by bacterium infections and the like. The action relating to moxalactam may be closely related to a target protein (target gene) thereof, for example, a protein containing the amino acid sequence shown by SEQ ID NO: 36 or a homologous protein thereof or variants of them.

The disease relating to mupirocin means a disease to which mupirocin is applied or a disease corresponding to the side effect of mupirocin. Mupirocin is known as an antibacterial preparation for ear nose throat region. The disease to which mupirocin is applied is exemplified by eradication of intranasal methicillin-resistance Staphylococcus aureus (MRSA), and the like. On the other hand, mupirocin is exemplified by mild topical reaction (rhinitis like symptom, irritating sensation etc.) and the like. The action relating to mupirocin may be closely related to a target protein (target gene) thereof, for example, a protein containing the amino acid sequence shown by SEQ ID NO: 24 or a homologous protein thereof or variants of them.

The disease relating to nefopam means a disease to which nefopam is applied or a disease corresponding to the side effect of nefopam. Nefopam is known as a central skeleton muscle relaxants. The action of nefopam is exemplified by central skeletal muscle relaxing action, antidepressive action, analgesic action and the like. The action relating to nefopam may be closely related to a target protein (target gene) thereof, for example, a protein containing the amino acid sequence shown by SEQ ID NO: 19 or a homologous protein thereof or variants of them.

The disease relating to nicardipine means a disease to which nicardipine is applied or a disease corresponding to the side effect of nicardipine. Nicardipine is a Ca antagonist and are known as a depressor. The disease to which nicardipine is applied is exemplified by essential hypertension and the like. On the other hand, the side effect of nicardipine is exemplified by thrombocytopenia, liver dysfunction, jaundice and the like. The action relating to nicardipine may be closely related to a target protein (target gene) thereof, for example, a protein containing the amino acid sequence shown by SEQ ID NO: 54 or a homologous protein thereof or variants of them.

The disease relating to nimesulide means a disease to which nimesulide is applied or a disease corresponding to the side effect of nimesulide. Nimesulide is a COX-2 selective inhibitor are known as antipyretic•analgesic•anti-inflammatory agent. The disease to which nimesulide is applied is exemplified by chronic rheumatoid arthritis, osteoarthritis and the like. On the other hand, the side effect of nimesulide is exemplified by hepatopathy and the like. The action relating to nimesulide may be closely related to a target protein (target gene) thereof, for example, a protein containing the amino acid sequence shown by SEQ ID NO: 27 or a homologous protein thereof or variants of them.

The disease relating to norharman means a disease to which norharman is applied or a disease corresponding to the side effect of norharman. Norharman is known as a carcinogenic substance presented in cigarette smoke and heating food. The action relating to norharman may be closely related to a target protein (target gene) thereof, for example, a protein containing the amino acid sequence shown by SEQ ID NO: 45 or a homologous protein thereof or variants of them.

The disease relating to oxytocin means a disease to which oxytocini applied or a disease corresponding to the side effect of oxytocin. Oxytocin is known as a posterior pituitary hormone preparation. The disease to which oxytocin is applied is exemplified by induction and promotion of uterine contraction and treatment for uterine bleeding (induction of childbirth•seak pains•atonic bleeding•before and after delivery of the placenta•subinvolution of the uterus•Caesarean section•after delivery of fetus), abortion, artificial abortion and the like. On the other hand, the side effect of oxytocin is exemplified by shock, excessively strong pains (uterus rupture•cervical laceration•amniotic fluid embolism•seak pains•atonic bleeding etc.), fetal asphyxia and the like. The action relating to oxytocin may be closely related to a target protein (target gene) thereof, for example, a protein containing the amino acid sequence shown by SEQ ID NO: 49 or a homologous protein thereof or variants of them.

The disease relating to paroxetine means a disease to which paroxetine is applied or a disease corresponding to the side effect of paroxetine. Paroxetine is a selective serotonin reuptake inhibitor (SSRI) and are known as an antidepressant•a mood-stabilizing drug•a psychostimulant drug. The disease to which paroxetine is applied is exemplified by depression•state of depression, panic disorder and the like. On the other hand, the side effect of paroxetine is exemplified by nausea, somnolentia, dry mouth, dizziness, serotonin syndrome, malignant syndrome, confusion, twitch, syndrome of inappropriate secretion of anti-diuretic hormone (SIADH), severe liver dysfunction (liver failure•liver necrosis•hepatitis•jaundice etc.) and the like. The action relating to paroxetine may be closely related to a target protein (target gene) thereof, for example, a protein containing the amino acid sequence shown by SEQ ID NO: 1 or SEQ ID NO: 25 or a homologous protein thereof or variants of them.

The disease relating to perhexiline means a disease to which perhexiline is applied or a disease corresponding to the side effect of perhexiline. Perhexiline is a suppressant of membrane carnitine palmitoyl-transferase (CPT1) and a Ca ion blocker and is known as a antianginal drug. The disease to which perhexiline is applied is exemplified by intractable angina pectoris in inoperable coronary heart disease patients, coronary blood vessel regeneration stage, ventricular repolarization abnormality and the like. On the other hand, the side effect of perhexiline is exemplified by electrocardiogram abnormality, ventricular repolarization abnormality, sinus bradycardia, prolonged QT interval, extrasystole, torsade de pointes, unconsciousness, headache, tremor, scotodinia, feeling of weakness, depression, fatigue, dizziness, peripheral nerve disorders, perception abnormality, body weight decrease, multipleneuropathy, sensorimotor neuropathy, congestion nipple, Guillain-Barre syndrome, ataxia, Parkinson's symptom, hypoglycemia, hyperinsulinemia, nausea, vomiting, eating disorder, upper abdominal pain, body weight decrease, cirrhosis, hepatic encephalopathy, portal veinhypertension, hepatitis, hepatic tumor, jaundice, keratopathy, bronchial cancer, bronchospasm, rash, muscle disorder and the like. The action relating to perhexiline may be closely related to a target protein (target gene) thereof, for example, a protein containing the amino acid sequence shown by SEQ ID NO: 23 or SEQ ID NO: 36 or a homologous protein thereof or variants of them.

The disease relating to phenformin means a disease to which phenformin is applied or a disease corresponding to the side effect of phenformin. Phenformin is known as a biguanide oral hypoglycemic drug. The disease to which phenformin is applied is exemplified by insulin-nondependent type diabetes and the like. On the other hand, the side effect of phenformin is exemplified by severe lactic acid acidosis or hypoglycemia and the like. The action relating to phenformin may be closely related to a target protein (target gene) thereof, for example, a protein containing the amino acid sequence shown by SEQ ID NO: 36 or a homologous protein thereof or variants of them.

The disease relating to pimethixene means a disease to which pimethixene is applied or a disease corresponding to the side effect of pimethixene. Pimethixene is known as an anti-histamine drugs. The action of pimethixene is exemplified by bronchial expand action, hypnotic•sedative action, anti-anxiety action and the like. The action relating to pimethixene may be closely related to a target protein (target gene) thereof, for example, a protein containing the amino acid sequence shown by SEQ ID NO: 1 or a homologous protein thereof or variants of them.

The disease relating to piperlongumine means a disease to which piperlongumine is applied or a disease corresponding to the side effect of piperlongumine. Piperlongumine is known as an alkaloid contained in root of piper longum. The action of piperlongumine is exemplified by anticonvulsant action and the like. The action relating to piperlongumine may be closely related to a target protein (target gene) thereof, for example, a protein containing the amino acid sequence shown by SEQ ID NO: 22 or a homologous protein thereof or variants of them.

The disease relating to pirenzepine means a disease to which pirenzepineis applied or a disease corresponding to the side effect of pirenzepine. Pirenzepine is a selective muscarine receptor antagonist and is known as a therapeutic drug for peptic ulcera (antacid). The disease to which pirenzepineis applied is exemplified by gastric mucosal lesion (erosion•haemorrhagia•redness•attached mucosa) in acute aggravation phase of acute gastritis•chronic gastritis and improvement of digestive symptom, upper gastrointestinal hemorrhage caused by gastric ulcer•duodenal ulcer, peptic ulcer•acute stress ulcer•acute stomach mucous membrane lesion, suppress of promotion of gastric secretion caused by operative stress, anesthetic premedication and the like. On the other hand, the side effect of pirenzepine is exemplified by dry mouth, constipation, diarrhea, rash, nausea, agranulocytosis, anaphylactoid symptoms and the like. The action relating to pirenzepinemay be closely related to a target protein (target gene) thereof, for example, a protein containing the amino acid sequence shown by SEQ ID NO: 40 or a homologous protein thereof or variants of them.

The disease relating to probenecid means a disease to which probenecid is applied or a disease corresponding to the side effect of probenecid. Probenecid is an uricosuric drug and is known as a therapeutic drug for gout•hyperuricemia. The disease to which probenecid is applied is exemplified by gout, maintain in blood concentration of penicillin•p-aminosalicylic acid, and the like. On the other hand, the side effect of probenecid is exemplified by anorexia, gastric distress, dermatitis, hemolytic anemia, aplastic anemia, anaphylactoid reaction, liver necrosis, nephrosissyndrome and the like. The action relating to probenecid may be closely related to a target protein (target gene) thereof, for example, a protein containing the amino acid sequence shown by SEQ ID NO: 23 or SEQ ID NO: 59 or a homologous protein thereof or variants of them.

The disease relating to procaine means a disease to which procaine is applied or a disease corresponding to the side effect of procaine. Procaine is known as a topical anesthetic. The disease to which procaine is applied is exemplified by spinal anesthesia (lumbar anesthesia), epidural anesthesia, conduction anesthesia, infiltration anesthesia, epidural anesthesia and the like. On the other hand, the side effect of procaine is exemplified by shock, poisoning symptom (tremor•twitch etc.) and the like. The action relating to procaine may be closely related to a target protein (target gene) thereof, for example, a protein containing the amino acid sequence shown by SEQ ID NO: 61 or a homologous protein thereof or variants of them.

The disease relating to propranolol means a disease to which propranolol is applied or a disease corresponding to the sideeffect of propranolol. Propranolol is an adrenergic β receptor blocker and is known as a depressor. The disease to which propranolol is applied is exemplified by angina pectoris, extrasystole (supraventricular, ventricular), prophylaxis of paroxysmal tachycardia, atrial fibrillation with a rapid ventricular response (bradycardia effect), sinus tachysystole, fresh atrial fibrillation, prophylaxis of paroxysmal atrial fibrillation, melanocytoma surgery case, essential hypertension (mild—moderate disease) and the like. On the other hand, the side effect of propranolol is exemplified by circulatory (bradycardia, heartbeat number•cardiac rhythm disorder), dizziness, fall in blood pressure, congestive heart failure (or aggravation thereof), peripheral ischemia (Raynaud's symptom etc.), auriculoventricular block, orthostatic hypotension with faint, agranulocytosis, thrombocytopenia, purpura, bronchial spasm, dyspnea and the like. The action relating to propranolol may be closely related to a target protein (target gene) thereof, for example, a protein containing the amino acid sequence shown by SEQ ID NO: 22 or a homologous protein thereof or variants of them.

The disease relating to protriptyline means a disease to which protriptyline is applied or a disease corresponding to the side effect of protriptyline. Protriptyline is known as an antidepressant•mood-stabilizing drug•psychostimulant drug. The disease to which protriptyline is applied is exemplified by depressive symptom, sleep apnea, narcolepsy and the like. On the other hand, the side effect of protriptyline is exemplified by liver function alteration, body weight increase/decrease, sweating, eating disorder, epigastric urgency, diarrhea, anxiety, agitation, insomnia, panic disorder, ataxia, tremor, peripheral nerve disorders, perception paralysis, prick pain, bleary eyes, adjustment disorder, elevation of intraocular pressure, dilated pupil, confusional state, delusion, headache, nightmare, constipation, dry mouth, nausea, vomiting, impotent, hyposexuality, orthostatic hypotension, tachysystole, palpitation, perception abnormality, extrapyramidal symptom, sleepiness, dizziness, petechial hemorrhage, skin rash, urticaria, pruritus, photosensitization, tinnitus, brain wave change, feeling of weakness, fatigue, agranulocytosis, leucopenia, thrombocytopenia, purpura, myocardial infarction, cerebral apoplexy, cardiac block, arrhythmia, paralytic ileus, epilepsy and the like. The action relating to protriptyline may be closely related to a target protein (target gene) thereof, for example, a protein containing the amino acid sequence shown by SEQ ID NO: 63 or a homologous protein thereof or variants of them.

The disease relating to pyrilamine means a disease to which pyrilamine is applied or a disease corresponding to the side effect of pyrilamine. Pyrilamine is a H1 receptor antagonist and is known as an antiallergic agents. The disease to which pyrilamine is applied is exemplified by allergic disease and the like. On the other hand, the side effect of pyrilamine is exemplified by mild sedative action, strong anticholinergic action (nervousness, insomnia, convulsive attack, tremor, ataxia, dry mouth, eyesight disorder, urinary retention, constipation), palpitation, digestive system disorder, anorexia, feebleness, incoordination and the like. The action relating to pyrilamine may be closely related to a target protein (target gene) thereof, for example, a protein containing the amino acid sequence shown by SEQ ID NO: 36 or SEQ ID NO: 45 or a homologous protein thereof or variants of them.

The disease relating to quercetin means a disease to which quercetin is applied or a disease corresponding to the side effect of quercetin. Quercetin is a flavonoid contained in onion•citrus, and is known to have antiallergic action, anti-estrogen action, anticancer effect, antioxidant action and the like. The disease to which quercetin is applied is exemplified by mitigation of reaction for allergen, pollinosis, atopic dermatitis, palmoplantar pustulosis and the like. The action relating to quercetin may be closely related to a target protein (target gene) thereof, for example, a protein containing the amino acid sequence shown by SEQ ID NO: 20 or SEQ ID NO: 54 or a homologous protein thereof or variants of them.

The disease relating to quinacrine means a disease to which quinacrine is applied or a disease corresponding to the side effect of quinacrine. Quinacrine is a drug for parasite•protozoa and is known as a therapeutic drug for malaria. Furthermore, MAO inhibitory action is exemplified as an action of quinacrine. The disease to which quinacrine is applied is exemplified by giardiasis, cestode infection, malaria infections, amebiasis, collagen disease, pneumothorax, neoplastic effusion, female contraception and the like. On the other hand, the side effect of quinacrine is exemplified by aplastic anemia, blood coagulation lack, headache, dizziness, nightmare, irritability, nervousness, toxic psychosis, epilepsy, convulsion, nausea, eating disorder, diarrhea, abdomen convulsion, vomiting, hepatitis, corneal edema, retinopathy, interstitial pneumonia, granuloma, parachroma, rash, exfoliative reaction, skin atrophy, hair loss, pigmentary change, verruca formation, carcinoma planocellulare and the like. The action relating to quinacrine may be closely related to a target protein (target gene) thereof, for example, a protein containing the amino acid sequence shown by SEQ ID NO: 61 or a homologous protein thereof or variants of them.

The disease relating to quinine means a disease to which quinine is applied or a disease corresponding to the side effect of quinine. Quinine is a drug for parasite•protozo and is known as a therapeutic drug for malaria. The disease to which quinine is applied is exemplified by malaria infections and the like. On the other hand, the side effect of quinine is exemplified by blackwater fever (fever•hematuria•jaundice•intravascular hemolysis accompanying with acute renal failure and the like), amaurosis (accompanying with low vision•photophobia•central scotoma•field stenosis and the like which are caused by ophthalmic nerve disorder), thrombocytopenic purpura, agranulocytosis, hemolytic uremic syndrome and the like. The action relating to quinine may be closely related to a target protein (target gene) thereof, for example, a protein containing the amino acid sequence shown by SEQ ID NO: 1 or SEQ ID NO: 10 or a homologous protein thereof or variants of them.

The disease relating to rescinnamine means a disease to which rescinnamine is applied or a disease corresponding to the side effect of rescinnamine. Rescinnamine is a peripheral sympathetic blocking agent and is known as a depressor. The disease to which rescinnamine is applied is exemplified by essential hypertension, renal hypertension and the like. On the other hand, the side effect of rescinnamine is exemplified by state of depression, gastric ulcer, nightmare, extrapyramidal symptom, sleepiness, dizziness and the like. The action relating to rescinnamine may be closely related to a target protein (target gene) thereof, for example, a protein containing the amino acid sequence shown by SEQ ID NO: 41 or SEQ ID NO: 53 or a homologous protein thereof or variants of them.

The disease relating to risperidone means a disease to which risperidone is applied or a disease corresponding to the side effect of risperidone. Risperidone is a D₂ and 5-HT₂ antagonist and is known as an antipsychotic agent. The disease to which risperidone is applied is exemplified by schizophrenia and the like. On the other hand, the side effect of risperidone is exemplified by akathisia, insomnia, constipation, tremor, hypersalivation, sleepiness, anxiety, muscle rigidity, restlessness, malignant syndrome, tardive dyskinesia, paralytic ileus, syndrome of inappropriate secretion of anti-diuretic hormone, liver dysfunction, jaundice, rhabdomyolysis, arrhythmia, cerebrovascular disorder, elevated blood-glucose level and the like. The action relating to risperidone may be closely related to a target protein (target gene) thereof, for example, a protein containing the amino acid sequence shown by SEQ ID NO: 13 or SEQ ID NO: 35 or a homologous protein thereof or variants of them.

The disease relating to ritodrine means a disease to which ritodrine is applied or a disease corresponding to the side effect of ritodrine. Ritodrine is an adrenergic β₂ receptor stimulant and is known as a therapeutic drug for immature birth. The disease to which ritodrine is applied is exemplified by imminent abortion•immature birth and the like. On the other hand, the side effect of ritodrine is exemplified by palpitation, finger tremor, nausea, rhabdomyolysis, pancytopenia, decreased serum potassium level, neonatal intestinal obstruction and the like. The action relating to ritodrine may be closely related to a target protein (target gene) thereof, for example, a protein containing the amino acid sequence shown by SEQ ID NO: 1 or SEQ ID NO: 2 or a homologous protein thereof or variants of them.

The disease relating to saquinavir means a disease to which saquinavir is applied or a disease corresponding to the side effect of saquinavir. Saquinavir is a peptide-like synthetic substrate analog inhibiting HIV-1 and HIV-2 protease activity and is known as antiviral agent (a therapeutic drug for HIV infections) which inhibits production of infectious virus by inhibit of cleavage of precursor polyprotein by HIV protease. The disease to which saquinavir is applied is exemplified by combination therapy with nucleoside HIV reverse transcriptase inhibitor in acquired immunodeficiency syndrome (AIDS), and the like. On the other hand, the side effect of saquinavir is exemplified by anemia, increased blood glucose level, increased blood uric acid, eosinophilia, nausea, fever, digestive disorder (diarrhea, abdomen uncomfortable feeling, nausea, vomiting etc.), suicide attempt, twitch, poliomyelitis, spinal nerve root polyneuropathy, leukoencephalopathy, hallucination, confusion, pancreatitis, the intestine obstruct, severe liver dysfunction (jaundice, ascites, portal hypertension, curable cholangitis), thrombophlebitis, cyanosis, peripheral vasoconstriction, acute myeloblastic leukemia, pancytopenia, hemolytic anemia, thrombocytopenia, intracranial hemorrhage, hemoptysis, hemorrhagic diathesis, diabetes (aggravation thereof), hyperglycemia, ketoacidosis, skin mucocutaneous ocular syndrome, acute renal failure, kidney stone, tumor, multiplearthritis and the like. The action relating to saquinavir may be closely related to a target protein (target gene) thereof, for example, a protein containing the amino acid sequence shown by SEQ ID NO: 17 or SEQ ID NO: 53 or a homologous protein thereof or variants of them.

The disease relating to scoulerine means a disease to which scoulerine is applied or a disease corresponding to the side effect of scoulerine. Scoulerine is known as an alkaloid of Fumariaceae plant. The action of scoulerine is exemplified by hypnotic action, sedative action, antiemetic action and the like. The action relating to scoulerine may be closely related to a target protein (target gene) thereof, for example, a protein containing the amino acid sequence shown by SEQ ID NO: 2 or a homologous protein thereof or variants of them.

The disease relating to sulfadimethoxine means a disease to which sulfadimethoxine is applied or a disease corresponding to the side effect of sulfadimethoxine. Sulfadimethoxine is a kind of sulfa drug which is a structure analog of para-aminobenzoic acid and is known as a chemotherapeutic agent having bacterial growth inhibitory action by reversible inhibition of folic acid synthesis. The disease to which sulfadimethoxine is applied is exemplified by meningitis, pyelonephritis, cystitis, adenoiditis, pharyngitis, laryngitis, chancroid and the like. On the other hand, the side effect of sulfadimethoxine is exemplified by anorexia, nausea, vomiting, headache, shock, aplastic anemia, hemolytic anemia, skin mucocutaneous ocular syndrome, toxic epidermal necrosis and the like. The action relating to sulfadimethoxine may be closely related to a target protein (target gene) thereof, for example, a protein containing the amino acid sequence shown by SEQ ID NO: 1 or a homologous protein thereof or variants of them.

The disease relating to sulfaphenazole means a disease to which sulfaphenazoleis applied or a disease corresponding to the side effect of sulfaphenazole. Sulfaphenazole is a kind of sulfa drug which is a structure analog of para-aminobenzoic acid and is known as a chemotherapeutic agent having bacterial growth inhibitory action by reversible inhibition of folic acid synthesis. The disease to which sulfaphenazoleis applied is exemplified by meningitis, pyelonephritis, cystitis, adenoiditis, pharyngitis, laryngitis, chancroid and the like. On the other hand, the side effect of sulfaphenazole is exemplified by anorexia, nausea, vomiting, headache, shock, aplastic anemia, hemolytic anemia, skin mucocutaneous ocular syndrome, toxic epidermal necrosis and the like. The action relating to sulfaphenazole may be closely related to a target protein (target gene) thereof, for example, a protein containing the amino acid sequence shown by SEQ ID NO: 23 or a homologous protein thereof or variants of them.

The disease relating to syrosingopine means a disease to which syrosingopine is applied or a disease corresponding to the side effect of syrosingopine. Syrosingopine is known as a depressor. The disease to which syrosingopine is applied is exemplified by essential hypertension, hypotensive action, sedative action and the like. On the other hand, the side effect of syrosingopine is exemplified by gastric ulcer, nasal congestion, sleepiness, dizziness, dry mouth, drug-induced depressive state, suicide and the like. The action relating to syrosingopine may be closely related to a target protein (target gene) thereof, for example, a protein containing the amino acid sequence shown by SEQ ID NO: 53 or a homologous protein thereof or variants of them.

The disease relating to tamoxifen means a disease to which tamoxifen is applied or a disease corresponding to the side effect of tamoxifen. Tamoxifen has an anti-estrogen action by competitive binding to estrogen against estrogen receptor such as breast cancer tissue and is known as an anti-cancer agent. The disease to which tamoxifen is applied is exemplified by breast cancer and the like. On the other hand, the side effect of tamoxifen is exemplified by amenorrhea, menstrual disorder, nausea, vomiting, anorexia, leucopenia, anemia, thrombocytopenia, eyesight abnormality, vision disorder, embolized thrombus, phlebitis, hepatopathy, hypercalcemia, hysteromyoma, endometrial polyp, endometrial hyperplasia, endometriosis, interstitial pneumonia, anaphylactoid symptoms, skin mucocutaneous ocular syndrome, bullous pemphigoid, pancreatitis and the like. The action relating to tamoxifen may be closely related to a target protein (target gene) thereof, for example, a protein containing the amino acid sequence shown by SEQ ID NO: 3 or a homologous protein thereof or variants of them.

The disease relating to terconazole means a disease to which terconazole is applied or a disease corresponding to the side effect of terconazole. Terconazole is known as a triazole antifungal agent. The disease to which terconazole is applied is exemplified by fungus infection, vaginal infection and the like. The action relating to terconazole may be closely related to a target protein (target gene) thereof, for example, a protein containing the amino acid sequence shown by SEQ ID NO: 36 or a homologous protein thereof or variants of them.

The disease relating to thioproperasine means a disease to which thioproperasine is applied or a disease corresponding to the side effect of thioproperasine. Thioproperasine is known as an antipsychotic agents. The disease to which thioproperasine is applied is exemplified by schizophrenia and the like. On the other hand, the side effect of thioproperasine is exemplified by malignant syndrome, extrapyramidal symptom, Parkinson's syndrome(finger tremor, muscle rigidity, hypersalivation etc.), dyskinesia (spasmodic torticollis, facial and neck contraction, opisthotonus, eyeballrpm attack etc.), akathisia, involuntary movement around mouth and the like, body weight increase, gynecomastia, milk secretion, aspermatism, menstrual disorder, glucosuria, psychoneurosis: derangement, insomnia, headache, anxiety, agitation, irritability, dry mouth, congested nose, feebleness, fever, edema, urinary retention, anuresis, frequent urination, incontinence, pigmentation of skin, systemic lupus erythematosus and the like. The action relating to Thioproperasine may be closely related to a target protein (target gene) thereof, for example, a protein containing the amino acid sequence shown by SEQ ID NO: 23 or SEQ ID NO: 27 or a homologous protein thereof or variants of them.

The disease relating to thiothixene(cis) means a disease to which thiothixene(cis) is applied or a disease corresponding to the side effect of thiothixene(cis). Thiothixene(cis) is known as an antipsychotic agents. The disease to which thiothixene(cis) is applied is exemplified by schizophrenia and the like. On the other hand, the side effect of thiothixene(cis) is exemplified by circulatory collapse, comatose states, sleepiness, dizziness, tardive dyskinesias, hyperreflexia, dry mouth, sweating, liver dysfunction, vision disorder and the like. The action relating to thiothixene(cis) may be closely related to a target protein (target gene) thereof, for example, a protein containing the amino acid sequence shown by SEQ ID NO:23 or a homologous protein thereof or variants of them.

The disease relating to tobramycin means a disease to which tobramycin is applied or a disease corresponding to the side effect of tobramycin. Tobramycin is known as an aminoglycoside antibiotic having inhibitory action of bacterial protein synthesis. The disease to which tobramycin is applied is exemplified by infections caused by pseudomonas•myxomycete and infections caused by klebsiella•Escherichia coli•enterobacter (sepsis, subcutaneous abscess, furuncle, cellulitis, post-operative wound infections, bronchitis, infection in bronchiectasis, pneumonia, peritonitis, pyelonephritis, cystitis, eyelid inflammation, dacryocystitis, hordeolum, conjunctivitis, keratitis, corneal ulcer and the like. The action relating to tobramycin may be closely related to a target protein (target gene) thereof, for example, a protein containing the amino acid sequence shown by SEQ ID NO: 36 or a homologous protein thereof or variants of them.

The disease relating to tolbutamide means a disease to which tolbutamide is applied or a disease corresponding to the side effect of tolbutamide. Tolbutamide is known as an oral sulfonylurea hypoglycemic drug. The disease to which tolbutamide is applied is exemplified by insulin-nondependent type diabetes and the like. On the other hand, the side effect of tolbutamide is exemplified by hypoglycemia, aplastic anemia, hemolytic anemia, agranulocytosis and the like. The action relating to tolbutamide may be closely related to a target protein (target gene) thereof, for example, a protein containing the amino acid sequence shown by SEQ ID NO: 23 or a homologous protein thereof or variants of them.

The disease relating to trifluoperazine means a disease to which trifluoperazine is applied or a disease corresponding to the side effect of trifluoperazine. Trifluoperazine is known as a phenothiazine therapeutic drug for schizophrenia. The disease to which trifluoperazine is applied is exemplified by schizophrenia and the like. On the other hand, the side effect of trifluoperazine is exemplified by malignant syndrome, sudden death, hypotensive, electrocardiogram abnormality (prolonged QT interval, flattening or inversion of T-wave, appearance of bimodal T-wave or U-wave etc.), paralytic ileus, tardive dyskinesia, ophthalmopathy (possibility of opacity of cornea•crystal and dye sedimentation of retina•cornea by long-term or large dose of administration), syndrome of inappropriate secretion of anti-diuretic hormone, aplastic anemia, SLE-like symptom, and the like. The action relating to trifluoperazine may be closely related to a target protein (target gene) thereof, for example, a protein containing the amino acid sequence shown by SEQ ID NO: 34 or a homologous protein thereof or variants of them.

The disease relating to trimetazidine means a disease to which trimetazidine is applied or a disease corresponding to the side effect of trimetazidine. Trimetazidine is a coronary vasodilator and is known as an antianginal drug. The disease to which trimetazidine is applied is exemplified by angina pectoris, myocardial infarction (excluding acute phase), other ischemic cardiac diseases and the like. On the other hand, the side effect of trimetazidine is exemplified by nausea, digestive symptom (gastric distress•anorexia etc.), psychological•neurological symptom (headache•feebleness•lightheadedness etc.), skin symptom (rash etc.) and the like. The action relating to trimetazidine may be closely related to a target protein (target gene) thereof, for example, a protein containing the amino acid sequence shown by SEQ ID NO: 5 or a homologous protein thereof or variants of them.

The disease relating to viloxazine means a disease to which viloxazine is applied or a disease corresponding to the side effect of viloxazine. Viloxazine is known as an antidepressant•mood-stabilizing drug•psychostimulant drug. The disease to which viloxazine is applied is exemplified by anxiety, depression, enuresis, narcolepsy, dysthymia and the like. On the other hand, the side effect of viloxazine is exemplified by nausea, vomiting, insomnia, anorexia, upper abdominal pain, diarrhea, constipation, dizziness, orthostatic hypotension, lower leg edema, articulation disorder, psychomotor agitation, delirium tremens, inappropriate secretion of antidiuretic hormone, attack, satyromania and the like. The action relating to viloxazine may be closely related to a target protein (target gene) thereof, for example, a protein containing the amino acid sequence shown by SEQ ID NO: 58 or a homologous protein thereof or variants of them.

The disease relating to xylazine means a disease to which xylazine is applied or a disease corresponding to the side effect of xylazine. Xylazine is an α₂ receptor agonist and is known as a sedative hypnotic (mainly animal drug). The disease to which xylazine is applied is exemplified by sedation, anesthesia, analgesic, muscle relation and the like. On the other hand, the side effect of xylazine is exemplified by bradycardia•low blood pressure•conductive disorder•cardiac muscle suppress and the like. The action relating to xylazine may be closely related to a target protein (target gene) thereof, for example, a protein containing the amino acid sequence shown by SEQ ID NO: 8 or a homologous protein thereof or variants of them.

The disease relating to acetylsalicylsalicylic acid means a disease to which acetylsalicylsalicylic acid is applied or a disease corresponding to the side effect of acetylsalicyl salicyl acid. Acetylsalicylsalicylic acid is known as an impurity contained in acetylsalicylic acid which is an antipyretic•analgesic•anti-inflammatory agent. The action relating to acetylsalicylsalicylacid may be closely related to a target protein (target gene) thereof, for example, a protein containing the amino acid sequence shown by SEQ ID NO: 28 or a homologous protein thereof or variants of them.

The disease relating to nimetazepam means a disease to which nimetazepam is applied or a disease corresponding to the side effect of nimetazepam. Nimetazepam is known as a benzodiazepine sedative hypnotic. The disease to which nimetazepam is applied is exemplified by insomnia and the like. On the other hand, the side effect of nimetazepam is exemplified by drug dependency, abstinence symptom caused by large dose of administration, or acute decrease of dose or withdrawal during consecutive use (convulsive attack, deliria, tremor, insomnia, anxiety, hallucination, delusion etc.), stimulation, confusion and the like. The action relating to nimetazepam may be closely related to a target protein (target gene) thereof, for example, a protein containing the amino acid sequence shown by SEQ ID NO: 25 or a homologous protein thereof or variants of them.

The disease relating to clobazam means a disease to which clobazam is applied or a disease corresponding to the side effect of clobazam. Clobazam is known as a benzodiazepine anticonvulsant. The disease to which clobazam is applied is exemplified by combination use with other anticonvulsant in partial seizure and generalized seizure, and the like. On the other hand, the side effect of clobazam is exemplified by sleepiness, dizziness, ambiopia, anorexia, drug dependence caused by consecutive use in large amounts, respiratory depression, increase of expectoration, airway hypersecretion, leucopenia, eosinophils increase, thrombocytopenia and the like. The action relating to clobazam may be closely related to a target protein (target gene) thereof, for example, a protein containing the amino acid sequence shown by SEQ ID NO: 48 or a homologous protein thereof or variants of them.

The disease relating to alimemazine means a disease to which alimemazine is applied or a disease corresponding to the side effect of alimemazine. Alimemazine is known as a phenothiazine anti-histamine drugs. The disease to which alimemazine is applied is exemplified by itching accompanied by dermatic diseases (eczema, skin itching, strophulus infantum, intoxication dermatosis, bite and stab wound), urticarial eruption, sneeze•nasal mucus•coughing accompanied by upper respiratory infection such as cold and the like, allergic rhinitis and the like. On the other hand, the side effect of alimemazine is exemplified by rash, agranulocytosis, sleepiness, dizziness, feebleness, headache, dry mouth and the like. The action relating to alimemazine may be closely related to a target protein (target gene) thereof, for example, a protein containing the amino acid sequence shown by SEQ ID NO: 1 or SEQ ID NO: 2 or a homologous protein thereof or variants of them.

The disease relating to tranilast means a disease to which tranilast is applied or a disease corresponding to the side effect of tranilast. Tranilast is known as an antiallergic agent having chemical mediator release suppressive action. The disease to which tranilast is applied is exemplified by bronchial asthma, allergic rhinitis, allergic conjunctivitis, atopic dermatitis, keloid•hyperplastic scar and the like. On the other hand, the side effect of tranilast is exemplified by cystitis-like symptom (frequent urination, urination pain, hematuria, feeling of residual urine etc.), liver dysfunction (jaundice, hepatitis), kidney dysfunction, leucopenia, thrombocytopenia and the like. The action relating to tranilast may be closely related to a target protein (target gene) thereof, for example, a protein containing the amino acid sequence shown by SEQ ID NO: 32 or a homologous protein thereof or variants of them.

The disease relating to ebastine means a disease to which ebastine is applied or a disease corresponding to the side effect of ebastine. Ebastine is known as a histamine H₁ receptor antagonist. The disease to which ebastine is applied is exemplified by urticarial eruption, eczema•dermatitis, prurigo, skin itching, allergic rhinitis and the like. On the other hand, the side effect of ebastine is exemplified by shock, anaphylactoid symptoms, liver dysfunction, jaundice and the like. The action relating to ebastine may be closely related to a target protein (target gene) thereof, for example, a protein containing the amino acid sequence shown by SEQ ID NO: 54 or a homologous protein thereof or variants of them.

The disease relating to pranlukast means a disease to which pranlukast is applied or a disease corresponding to the side effect of pranlukast. Pranlukast is known as an antiallergic agent having leukotriene antagonistic action. The disease to which pranlukast is applied is exemplified by bronchial asthma, allergic rhinitis and the like. On the other hand, the side effect of pranlukast is exemplified by abdominal pain•gastric distress, diarrhea, heart burn, liver dysfunction, increased bilirubin, rash•itching and the like, shock•anaphylactoid symptoms, leucopenia, thrombocytopenia, interstitial pneumonia•eosinophilic pneumonia, rhabdomyolysis, acute renal failure caused by rhabdomyolysis and the like. The action relating to pranlukast may be closely related to a target protein (target gene) thereof, for example, a protein containing the amino acid sequence shown by SEQ ID NO: 18, SEQ ID NO: 20, SEQ ID NO: 21, SEQ ID NO: 35, SEQ ID NO: 42, SEQ ID NO: 53 or SEQ ID NO: 54 or a homologous protein thereof or variants of them.

The disease relating to methyclothiazide means a disease to which methyclothiazide is applied or a disease corresponding to the side effect of methyclothiazide. Methyclothiazide is known as a thiazido diuretic. The disease to which methyclothiazide is applied is exemplified by edema (including congestive heart failure)•diuretic action in hypertension, and the like. On the other hand, the side effect of methyclothiazide is exemplified by hypokalemia, hyperuricemia, impaired glucose tolerance, hypercholesterolemia, hypertriglyceridemia, hypercalcemia, male sexual dysfunction, weakness, rash and the like. The action relating to methyclothiazide may be closely related to a target protein (target gene) thereof, for example, a protein containing the amino acid sequence shown by SEQ ID NO: 16 or SEQ ID NO: 23 or a homologous protein thereof or variants of them.

The disease relating to alacepril means a disease to which alacepril is applied or a disease corresponding to the side effect of alacepril. Alacepril is an angiotensin-converting enzyme (ACE) inhibitor and is known as a depressor. The disease to which alacepril is applied is exemplified by essential hypertension, renal hypertension and the like. On the other hand, the side effect of alacepril is exemplified by angioedema (angioedema accompanying with dyspnea, which has a symptom of tumentia in face, tongue, glottis, larynx), agranulocytosis, pemphigus-like symptom, hyperkalemia and the like. The action relating to alacepril may be closely related to a target protein (target gene) thereof, for example, a protein containing the amino acid sequence shown by SEQ ID NO: 24 or a homologous protein thereof or variants of them.

The disease relating to clinofibrate means a disease to which clinofibrate is applied or a disease corresponding to the side effect of clinofibrate. Clinofibrate is known as a fibrate therapeutic drug for hyperlipidemia. The disease to which clinofibrate is applied is exemplified by hyperlipidemia and the like. On the other hand, the side effect of clinofibrate is exemplified by rhabdomyolysis and the like. The action relating to clinofibrate may be closely related to a target protein (target gene) thereof, for example, a protein containing the amino acid sequence shown by SEQ ID NO: 23 or SEQ ID NO: 34 or a homologous protein thereof or variants of them.

The disease relating to acetylcysteine means a disease to which acetylcysteine is applied or a disease corresponding to the side effect of acetylcysteine. Acetylcysteine has a mucolysis action and is known as airway mucolysis agent, thus, expectorant. The disease to which acetylcysteine is applied is exemplified by detoxication in excess ingestion of acetaminophen, expectoration in the following disease (bronchial asthma, chronic bronchitis, bronchiectasis, pulmonary tuberculosis, emphysema, upper respiratory infection, lung suppuration, pneumonia, cystic fibrosis), before and after treatment of the following (bronchography, bronchoscopy, lung cancer cytologic diagnosis, tracheostomy) and the like. On the other hand, the side effect of acetylcysteine is exemplified by bronchial obstruct, bronchial spasm and the like. The action relating to acetylcysteine may be closely related to a target protein (target gene) thereof, for example, a protein containing the amino acid sequence shown by SEQ ID NO: 2 or SEQ ID NO: 3 or a homologous protein thereof or variants of them.

The disease relating to buformin means a disease to which buformin is applied or a disease corresponding to the side effect of buformin. Buformin is known as a biguanide oral hypoglycemic drug. The disease to which buformin is applied is exemplified by insulin-nondependent type diabetes and the like. On the other hand, the side effect of buformin is exemplified by severe lactic acid acidosis or hypoglycemia and the like. The action relating to buformin may be closely related to a target protein (target gene) thereof, for example, a protein containing the amino acid sequence shown by SEQ ID NO: 57 or a homologous protein thereof or variants of them.

The disease relating to terguride means a disease to which terguride is applied or a disease corresponding to the side effect of terguride. Terguride is known as a ergot alkaloid sustained dopamine agonist. The disease to which terguride is applied is exemplified by hyperprolactinemic ovulation disorder, hyperprolactinemic pituitary gland adenoma, galactorrhea, puerperal milk secretion suppress and the like. On the other hand, the side effect of terguride is exemplified by shock caused by acute lowering of blood pressure, fibrotic change in pleura or lung accompanying with coughing•dyspnea, hallucination•delusion, deliria, aggravation of stomach•duodenal ulcer, and the like. The action relating to terguride may be closely related to a target protein (target gene) thereof, for example, a protein containing the amino acid sequence shown by SEQ ID NO: 9 or a homologous protein thereof or variants of them.

The disease relating to stanozolol means a disease to which stanozolol is applied or a disease corresponding to the side effect of stanozolol. Stanozolol is a testosterone derivative and is known as a synthesized anabolic hormone. The disease to which stanozolol is applied is exemplified by osteoporosis, pituitary gland dwarfism, debilitating state in chronic renal diseases•malignant tumor•post-operative•trauma•burn, bone marrow debilitating state in aplastic anemia, hereditary angioedema, muscle growth insufficiency and the like. On the other hand, the side effect of stanozolol is exemplified by jaundice, hoarseness•hypertrichiasis•acne•dye deposition•menstrual disorder•clitoral hypertrophy•aphrodisia in female, acne•penile enlargement in male, impotence, sustained erection, sperm decrease•semen decrease caused by continuation in a large dose, anaphylaxis and the like. The action relating to stanozolol may be closely related to a target protein (target gene) thereof, for example, a protein containing the amino acid sequence shown by SEQ ID NO: 16 or a homologous protein thereof or variants of them.

The disease relating to mestanolone means a disease to which mestanolone is applied or a disease corresponding to the side effect of mestanolone. Mestanolone is known as an anabolic hormone. The disease to which mestanolone is applied is exemplified by osteoporosis, pituitary dwarfism, remarkable debilitating state in chronic renal diseases•malignant tumor•post-operative•trauma•burn, and the like. On the other hand, the side effect of mestanolone is exemplified by hepatopathy (increase of GOT•GPT, delay of BSP excretion etc.), female endocrine disturbance (hoarseness, hypertrichiasis, acne, dye deposition, menstrual disorder, clitoral hypertrophy, aphrodisia in female), male endocrine disturbance (acne•penile enlargement, impotence, sustained erection, orchis function suppress caused by continuation administration in a large dose, sperm decrease•semen decrease in male) and the like. The action relating to mestanolone may be closely related to a target protein (target gene) thereof, for example, a protein containing the amino acid sequence shown by SEQ ID NO: 42 or a homologous protein thereof or variants of them.

The disease relating to pantethine means a disease to which pantethine is applied or a disease corresponding to the side effect of pantethine. Pantethine is a vitamin B₅ (pantothenic acid) preparation and is known as metabolism abnormality improving agent. The disease to which pantethine is applied is exemplified by prophylaxis and treatment for pantothenic acid deficiency (debilitating disease, hyperthyroidism, for pregnant women, nursing woman and the like), following diseases which are considered to be involved to lack or metabolism disorder of pantothenic acid (hyperlipidemia, atonic constipation, post-operative intestine paralysis, prophylaxis and treatment of side effect caused by streptomycin and kanamycin, acute•chronic eczema, improvement of platelet number and hemorrhagic tendency in blood diseases) and the like. On the other hand, the side effect of pantethine is exemplified by abdominal distension, abdominal pain, diarrhea•loose stool, nausea and the like. The action relating to pantethine may be closely related to a target protein (target gene) thereof, for example, a protein containing the amino acid sequence shown by SEQ ID NO: 1 or a homologous protein thereof or variants of them.

The disease relating to limaprost means a disease to which limaprost is applied or a disease corresponding to the side effect of limaprost. Limaprost is a prostaglandin E1 derivative and is known as a platelet coagulation suppressant, thus, antithrombotic agent. The disease to which limaprost is applied is exemplified by improvement of ulcer•pain accompanied by obstructive thromboangiitis and various ischemic symptoms such as cold feeling, and the like, and improvement of subjective symptoms (lower leg pain, lower leg numbness) accompanied by acquired lumbar canal stenosis and walking ability, and the like. On the other hand, the side effect of limaprost is exemplified by gastric distress, rash, headache•heviness of the head, diarrhea, anemia, uterine contraction action has been reported in animal experiments (pregnant monkey•pregnant rat intravenous injection), and the like. The action relating to limaprost may be closely related to a target protein (target gene) thereof, for example, a protein containing the amino acid sequence shown by SEQ ID NO: 24 or a homologous protein thereof or variants of them.

The disease relating to sarpogrelate means a disease to which sarpogrelate is applied or a disease corresponding to the side effect of sarpogrelate. Sarpogrelate is known as a platelet coagulation suppressant, thus, an antithrombotic agent. The disease to which sarpogrelate is applied is exemplified by improvement of various ischemic symptoms such as ulcer•pain•cold feeling which are accompanied by chronic arterial obstruction, and the like. On the other hand, the side effect of sarpogrelate is exemplified by nausea, heartburn, abdominal pain, cerebral hemorrhage, gastrointestinal hemorrhage, thrombocytopenia, liver dysfunction, jaundice and the like. The action relating to sarpogrelate may be closely related to a target protein (target gene) thereof, for example, a protein containing the amino acid sequence shown by SEQ ID NO: 27 or a homologous protein thereof or variants of them.

The disease relating to aragatroban means a disease to which aragatroban is applied or a disease corresponding to the side effect of aragatroban. Aragatroban is known as an antithrombotic agent having anti-thrombin action. The disease to which aragatroban is applied is exemplified by improvement of neural symptoms (movement paralysis) and daily life behavior (walking, standing up, sitting position maintenance, diet) which are accompanied by brain thrombosis acute stage within 48 hr of onset, improvement of limb ulcer•pain at rest in chronic arterial obstruction (Buerger's disease•obstructive arteriosclerosis) and cold feeling, inhibiting of coagulation of perfused blood during blood extracorporeal circulation in congenital antithrombin III deficient patients and patients with decreased antithrombin III (hemodialysis patients), and the like. On the other hand, the side effect of aragatroban is exemplified by hemorrhagic cerebral infarction, cerebral hemorrhage, gastrointestinal hemorrhage, shock•anaphylactic shock, fulminant hepatitis and the like. The action relating to aragatroban may be closely related to a target protein (target gene) thereof, for example, a protein containing the amino acid sequence shown by SEQ ID NO: 23 or a homologous protein thereof or variants of them.

The disease relating to fludroxycortide means a disease to which fludroxycortide is applied or a disease corresponding to the side effect of fludroxycortide. Fludroxycortide is a adrenal corticosteroid and is known as an external antiphlogistic•analgesia•antipruritic agent. The disease to which fludroxycortide is applied is exemplified by eczema•dermatitis (including keratodermia tylodes palmaris progressiva, lichen Vidal), nodular prurigo (including urticaria perstans), psoriasis, palmoplantar pustulosis, lichen ruber planus, amyloid lichen, cyclic granuloma, gloss lichen, chronic discoid lupus erythematodes, morbus Fox-Fordyce, hyperplastic scar•keloid, vitiligo vulgaris, Schamberg disease, malignant lymphoma (erythema•flat infiltration stage of mycosis fungoides etc.) and the like. On the other hand, the side effect of fludroxycortide is exemplified by hypertonia oculi•glaucoma•posterior subcapsular cataract and the like wherein immunity suppress action possibly aggravate infection. The action relating to fludroxycortide may be closely related to a target protein (target gene) thereof, for example, a protein containing the amino acid sequence shown by SEQ ID NO: 25 or a homologous protein thereof or variants of them.

The disease relating to sulfadoxine means a disease to which sulfadoxine is applied or a disease corresponding to the side effect of sulfadoxine. Sulfadoxine is a sulfa drug and is known as a therapeutic drug for malaria. The disease to which sulfadoxine is applied is exemplified by malaria infections and the like. On the other hand, the side effect of Sulfadoxine is exemplified by skin mucocutaneous ocular syndrome, toxic epidermal necrosis, PIE syndrome, hepatocyte necrosis, hemolytic anemia, pancytopenia, hypoglycemic state by enhance of hypoglycemic action caused by glibenclamide and the like, and the like. The action relating to sulfadoxine may be closely related to a target protein (target gene) thereof, for example, a protein containing the amino acid sequence shown by SEQ ID NO: 23 or a homologous protein thereof or variants of them.

The disease relating to ubenimex means a disease to which ubenimex is applied or a disease corresponding to the side effect of ubenimex. Ubenimex is known as a non-specific anti-malignant tumor agent. The disease to which ubenimex is applied is exemplified by prolonged survival time in combination with chemotherapeutic agent to maintain and reinforce after induction of complete remission in adult acute nonlymphocytic leukemia, and the like. On the other hand, the side effect of ubenimex is exemplified by liver disorder, skin disorder (rash•redness, itching sensation, hair loss etc.), digestive organ disorder (nausea•vomiting, anorexia etc.) and the like. The action relating to ubenimex may be closely related to a target protein (target gene) thereof, for example, a protein containing the amino acid sequence shown by SEQ ID NO: 23 or a homologous protein thereof or variants of them.

The disease relating to celecoxib means a disease to which celecoxib is applied or a disease corresponding to the side effect of celecoxib. Celecoxib is selective cyclooxygenase 2 (COX2) inhibitor, antipyretic•analgesic•anti-inflammatory agent, and also is known to have cancer cell proliferation inhibitory action. The disease to which celecoxib is applied is exemplified by pyretolysis•analgesia•anti-inflammation, osteoarthritis, rheumatoid arthritis, ankylosing spondylitis, dysmenorrheal, adenomatous colon polyp in familial adenomatous polyposis (FAP), and the like. On the other hand, the side effect of celecoxib is exemplified by cardiovascular thrombosis (myocardial infarction, cerebral infarction), digestion tract disorder (gastrointestinal hemorrhage, gastrointestinal tract ulcer, gastrointestinal tract perforations), contraindication: analgesia in coronary artery bypass operation (CABG) and the like. The action relating to celecoxib may be closely related to a target protein (target gene) thereof, for example, a protein containing the amino acid sequence shown by SEQ ID NO: 23 or a homologous protein thereof or variants of them.

The disease relating to 6-furfurylaminopurine means a disease to which 6-furfurylaminopurine is applied or a disease corresponding to the side effect of 6-furfurylaminopurine. 6-Furfurylaminopurine is known as a plant growth promoter kinetin (agrichemical). The disease to which 6-furfurylaminopurine is applied is exemplified by promoting action of cell division•differentiation•growth, and the like. The action relating to 6-furfurylaminopurine may be closely related to a target protein (target gene) thereof, for example, a protein containing the amino acid sequence shown by SEQ ID NO: 57 or a homologous protein thereof or variants of them.

The disease relating to solasodine means a disease to which solasodine is applied or a disease corresponding to the side effect of solasodine. Solasodine is known as an alkaloid having an anti-cancer action. The disease or action to which solasodine is applied is exemplified by contraceptive, anti-cancer action, anaphylaxy or insulin•shock, shock by burn, and the like. The action relating to solasodine may be closely related to a target protein (target gene) thereof, for example, a protein containing the amino acid sequence shown by SEQ ID NO: 24 or a homologous protein thereof or variants of them.

The disease relating to gossypol means a disease to which gossypol is applied or a disease corresponding to the side effect of gossypol. Gossypol is an ingredient contained in plant Gossypium arboreum, and is known to have actions such as an antibacterial action•insecticide action•male contraception action (inhibition of sperm movement)•antivirus action•anti-cancer action and the like. The disease to which gossypol is applied is exemplified by enhancement of an effect of chemotherapeutic agent and radiation therapy by inhibiting Bcl-2/xL protein in head and neck cancer and the like, and the like. The action relating to gossypol may be closely related to a target protein (target gene) thereof, for example, a protein containing the amino acid sequence shown by SEQ ID NO: or a homologous protein thereof or variants of them.

The disease relating to fluorocurarine chloride means a disease to which fluorocurarine chloride is applied or a disease corresponding to the side effect of fluorocurarine chloride. Fluorocurarine chloride is a selective sympathetic ganglion blocker and has a weak antagonistic activity against nicotinic receptor in myoneural junction, and is known as an antihypertensive agent. The action relating to fluorocurarine chloride may be closely related to a target protein (target gene) thereof, for example, a protein containing the amino acid sequence shown by SEQ ID NO: 10 or a homologous protein thereof or variants of them.

The disease relating to pempidine means a disease to which pempidine is applied or a disease corresponding to the side effect of pempidine. Pempidine is known as a depressor having ganglionic blocking action and central action. The disease to which pempidine is applied is exemplified by hypertension and the like. The action relating to pempidine may be closely related to a target protein (target gene) thereof, for example, a protein containing the amino acid sequence shown by SEQ ID NO: 57 or a homologous protein thereof or variants of them.

The disease relating to nitrarine means a disease to which nitrarine is applied or a disease corresponding to the side effect of nitrarine. Nitrarine is known as a caltrop alkaloid. The action of nitrarine is exemplified by hypotensive action, spasmolysis action, coronary artery vasodilating action, sedative action and the like. The action relating to nitrarine may be closely related to a target protein (target gene) thereof, for example, a protein containing the amino acid sequence shown by SEQ ID NO: 46 or SEQ ID NO: 57 or a homologous protein thereof or variants of them.

The disease relating to promazine means a disease to which promazine is applied or a disease corresponding to the side effect of promazine. Promazine is known as an antipsychotic agent. The disease to which promazine is applied is exemplified by schizophrenia, mania, depression and state of depression, sedative hypnotic in neurosis, and the like. On the other hand, the side effect of promazine is exemplified by extrapyramidal symptom (ataxia, spasm, torticollis), dry mouth, somnolentia, coma, low body temperature, respiratory collapse, leucopenia, jaundice, coagulation disorder, rash and the like. The action relating to promazine may be closely related to a target protein (target gene) thereof, for example, a protein containing the amino acid sequence shown by SEQ ID NO: 18 or a homologous protein thereof or variants of them.

The disease relating to sulfabenzamido means a disease to which sulfabenzamido is applied or a disease corresponding to the side effect of sulfabenzamido. Sulfabenzamido is a synthesized antibacterial agent and is known as an antifungal agents. The disease to which sulfabenzamido is applied is exemplified by fungus infection (mainly animal drug) and the like. The action relating to sulfabenzamido may be closely related to a target protein (target gene) thereof, for example, a protein containing the amino acid sequence shown by SEQ ID NO: 23 or a homologous protein thereof or variants of them.

The disease relating to althiazide means a disease to which althiazide is applied or a disease corresponding to the side effect of althiazide. Althiazide is known as a diuretic. The disease to which Althiazide is applied is exemplified by hypertension and the like. The action relating to Althiazide may be closely related to a target protein (target gene) thereof, for example, a protein containing the amino acid sequence shown by SEQ ID NO: 23 or a homologous protein thereof or variants of them.

The disease relating to α-ergocryptine means a disease to which α-ergocryptine is applied or a disease corresponding to the side effect of α-ergocryptine. α-Ergocryptine is known as a vasoconstrictor. The disease to which α-ergocryptine is applied is exemplified by accompanying symptom accompanied by head trauma sequelae, hypertension, Buerger's disease•obstructive arteriosclerosis•arterial embolus•thrombosis•Raynaud's disease and Raynaud's syndrome•acroasphyxia•chilblain•frost injury, peripheral circulation disorder accompanied by intermittent claudication, and the like. On the other hand, the side effect of α-ergocryptine is exemplified by digestive trouble, nausea•vomiting, anorexia, rash•itching, headache•heaviness of the head, dizziness, bradycardia, lowering of blood pressure, brain anemia-like symptom, flush face, feeling of hot flushes, palpitation, thorax uncomfortable feeling and the like. The action relating to α-ergocryptine may be closely related to a target protein (target gene) thereof, for example, a protein containing the amino acid sequence shown by SEQ ID NO: 23 or SEQ ID NO: 53 or a homologous protein thereof or variants of them.

The disease relating to ebselen means a disease to which ebselen is applied or a disease corresponding to the side effect of ebselen. Ebselen is a brain protection drug having an antioxidant action and is known as a therapeutic drug for acute stage—cerebral infarction. The disease to which ebselen is applied is exemplified by nerve cell disorder in acute stage—cerebral infarction, and the like. The action relating to ebselen may be closely related to a target protein (target gene) thereof, for example, a protein containing the amino acid sequence shown by SEQ ID NO: 6 or a homologous protein thereof or variants of them.

The disease relating to furaltadone means a disease to which furaltadone is applied or a disease corresponding to the side effect of furaltadone. Furaltadone is known as a nitrofuran antibiotic (mainly animal drug). The disease to which furaltadone is applied is exemplified by bacterial infections and the like. On the other hand, the side effect of furaltadone is exemplified by carcinogenic and mutagenic. The action relating to furaltadone may be closely related to a target protein (target gene) thereof, for example, a protein containing the amino acid sequence shown by SEQ ID NO: 10 or a homologous protein thereof or variants of them.

The disease relating to pyrithyldione means a disease to which pyrithyldione is applied or a disease corresponding to the side effect of pyrithyldione. Pyrithyldione is known as a hypnotic sedatives. The disease to which pyrithyldione is applied is exemplified by insomnia and the like. On the other hand, the side effect of pyrithyldione is exemplified by agranulocytosis and the like. The action relating to pyrithyldione may be closely related to a target protein (target gene) thereof, for example, a protein containing the amino acid sequence shown by SEQ ID NO: 55 or a homologous protein thereof or variants of them.

The disease relating to benzthiazide means a disease to which benzthiazide is applied or a disease corresponding to the side effect of benzthiazide. Benzthiazide is known as a diuretic. The disease to which benzthiazide is applied is exemplified by hypertension, edema (cardiac•renal•hepatic), gestational toxicosis, premenstrual tension and the like. The action relating to benzthiazide may be closely related to a target protein (target gene) thereof, for example, a protein containing the amino acid sequence shown by SEQ ID NO: 23 or SEQ ID NO: 51 or a homologous protein thereof or variants of them.

The disease relating to levobunolol means a disease to which levobunolol is applied or a disease corresponding to the side effect of levobunolol. Levobunolol is known as a therapeutic drug for glaucoma. The disease to which levobunolol is applied is exemplified by glaucoma, ocular hypertension disease and the like. On the other hand, the side effect of levobunolol is exemplified by conjunctival hyperemia, keratitis, bronchial spasm, respiratory failure, congestive heart failure, cerebrovascular disorder, asthmatic attack, systemic lupus erythematosus and the like. The action relating to levobunolol may be closely related to a target protein (target gene) thereof, for example, a protein containing the amino acid sequence shown by SEQ ID NO:44 or a homologous protein thereof or variants of them.

The disease relating to raloxifene means a disease to which raloxifene is applied or a disease corresponding to the side effect of raloxifene. Raloxifene is a tamoxifen derivative and has a estrogen receptor control action and a bone metabolism control action, and is known as a bone metabolism improving drug or a therapeutic drug for osteoporosis. The disease to which raloxifene is applied is exemplified by postmenopausal osteoporosis and the like. On the other hand, the side effect of raloxifene is exemplified by intravenous embolized thrombus and the like. The action relating to raloxifene may be closely related to a target protein (target gene) thereof, for example, a protein containing the amino acid sequence shown by SEQ ID NO: 37 or a homologous protein thereof or variants of them.

The disease relating to luteolin means a disease to which luteolin is applied or a disease corresponding to the side effect of luteolin. Luteolin is a kind of flavonoid contained in plant (perilla, garland chrysanthemum, green pepper, camomile and the like) having antioxidant action, and Known to have antiallergic action•anti-cancer action and the like. The disease and action to which luteolin is applied is exemplified by allergic disease such as atopic dermatitis•pollinosis, immunity enhancing action, anti-inflammatory action, sepsis suppress action, suppress action of fleck•freckle, anti-cancer action and the like. The action relating to luteolin may be closely related to a target protein (target gene) thereof, for example, a protein containing the amino acid sequence shown by SEQ ID NO: 20 or SEQ ID NO: 54 or a homologous protein thereof or variants of them. The disease relating to valdecoxib means a disease to which valdecoxib is applied or a disease corresponding to the side effect of valdecoxib. Valdecoxib is a selective cyclooxygenase 2 (COX2) inhibitor, antipyretic•analgesic•anti-inflammatory agent, and is also known to have cancer cell proliferation inhibitory action. The disease to which valdecoxib is applied is exemplified by osteoarthritis, rheumatoid arthritis, dysmenorrheal (menstrual pain) and the like. On the other hand, the side effect of valdecoxib is exemplified by thrombus disease (myocardial infarction, cerebral apoplexy and the like), digestive organ disorder (ulcer formation, haemorrhagia, perforation) and the like. The action relating to valdecoxib may be closely related to a target protein (target gene) thereof, for example, a protein containing the amino acid sequence shown by SEQ ID NO: 23 or a homologous protein thereof or variants of them.

The disease relating to carboprost means a disease to which carboprost is applied or a disease corresponding to the side effect of carboprost. Carboprost is known as an abortion pill. The disease to which carboprost is applied is exemplified by abortion or induction of uterine contraction in hydatidiform mole treatment, and the like. On the other hand, the side effect of carboprost is exemplified by palpitation, headache, rash, uterus pain, body temperature decrease, fleck, chest pain, thorax pressure, dyspnea, constipation, diarrhea, vomiting and the like. The action relating to carboprost may be closely related to a target protein (target gene) thereof, for example, a protein containing the amino acid sequence shown by SEQ ID NO: 24 or SEQ ID NO: 34 or a homologous protein thereof or variants of them.

The disease relating to gabexate means a disease to which gabexate is applied or a disease corresponding to the side effect of gabexate. Gabexate is a protease inhibitor and is known as a therapeutic drug for pancreatitis. The disease to which gabexate is applied is exemplified by acute aggravation stage of acute pancreatitis•chronic relapsing pancreatitis accompanying escape of proteolytic enzyme (trypsin, kallikrein, plasmin etc.), post-operative acute pancreatitis, diffuse intravascular coagulation and the like. On the other hand, the side effect of gabexate is exemplified by anaphylactic shock, blood vessel inner wall disorder, increased hemorrhagic tendency, granulocyte decrease, eosinophilia and the like. The action relating to gabexate may be closely related to a target protein (target gene) thereof, for example, a protein containing the amino acid sequence shown by SEQ ID NO: 23 or a homologous protein thereof or variants of them.

(Diseases or Conditions Associated with Target Gene Y)

“A disease or condition associated with target gene Y” refers to a disease or condition that can be caused as a result of a functional change (e.g., functional changes due to mutations (e.g., polymorphism)), or a change in the expression level, in target gene Y, or in a gene located downstream of target gene Y in the signal transduction system mediated by target gene Y (downstream gene). A functional change in target gene Y or a gene downstream thereof can be caused by, for example, a mutation (e.g., polymorphism) in the gene. Examples of the mutation include a mutation in the coding region, which promotes or suppresses a function of the gene, a mutation in the non-coding region, which promotes or suppresses the expression thereof, and the like. The change in the expression level include increases or reductions in the expression level. A disease or condition associated with target gene Y can be ameliorated or exacerbated by target protein Y.

“A function associated with a target protein Y (target gene Y)” means a function of the same kind as, or opposite kind to, the kind of a function that is actually exhibited by target protein Y. In other words, a function associated with a target protein Y is a function capable of ameliorating or exacerbating “a disease or condition associated with target protein Y”. Hence, “a function associated with a target protein Y” is a function for promoting or suppressing an immune reaction, and the like, if target protein Y is a factor that promotes an immune reaction and the like. Examples of the function associated with a target protein Y include the functions shown in Tables 2-1 to 2-20.

Since target gene Y is considered to mediate a wide variety of physiological functions in the body; as diseases or conditions associated with target protein Y, a very wide variety of diseases or conditions are supposed. One such example of the diseases or condition associated with target protein Y include disease or condition associated with the functions shown in Tables 2-1 to 2-20.

Other examples of the disease or condition associated with target protein Y are diseases or conditions postulated from the annotation of target protein Y and target gene Y. Those skilled in the art can postulate such diseases or conditions by identifying homologous proteins or genes by homology search, and subsequently extensively examining the functions of the proteins or genes or the diseases or conditions mediated thereby by a commonly known method. Various methods are available for annotation analysis. Described below are the results of annotation of target genes for bioactive substances in the present application, by various methods using the sequences of human proteins or genes representative of target proteins or genes for bioactive substances as query sequences.

### Amino Acid Analysis 1

### Homology Analysis by BLASTP

The calculation program used was blastall 2.2.6. The target databases used were swiss-prot: 196277 (2005.10.25), (Refseq)hs: 24139 (2005.09.15), (Refseq)mouse: 18457 (2005.09.15), and (Refseq)rat: 9252 (2005.09.15). The cutoff value was established at 1.00E-05. The following data were processed by filtering:

### For Swiss-prot:

Having a definition beginning with “ALU SUBFAMILY”
Having a definition beginning with “Alu subfamily”
Having a definition beginning with “!!!! ALU SUBFAMILY”
Having a definition beginning with “B-CELL GROWTH FACTOR PRECURSOR”
Having a definition including “NRK2”
Having a definition beginning with “PROLINE-RICH”
Having a definition beginning with “GLYCINE-RICH”
Having a definition beginning with “EXTENSIN PRECURSOR”
Having a definition beginning with “COLLAGEN”
Having a definition beginning with “100 KD”
Having a definition beginning with “RETROVIRUS-RELATED POL POLYPROTEIN”
Having a definition beginning with “CUTICLE COLLAGEN”
Having a definition beginning with “HYPOTHETICAL”
Having a definition beginning with “Hypothetical”
Having a definition beginning with “SALIVARY PROLINE-RICH ROTEIN”
Having a definition beginning with “IMMEDIATE-EARLY PROTEIN”
Having the accession number “P49646”

### For Ref-seq:

Having a definition beginning with “hypothetical protein FLJ”
Having a definition beginning with “KIAA”
Having a definition beginning with “hypothetical protein DKFZ”
Having a definition beginning with “DKFZ”
Having a definition beginning with “RIKEN cDNA”
Having a definition beginning with “hypothetical protein MGC”
Having a definition beginning with “hypothetical protein”
Having a definition beginning with “hypothetical protein PP”
Having a definition beginning with “neuronal thread protein”
Having a definition beginning with “clone FLB”
Having a definition beginning with “hypothetical protein PRO”
Having a definition beginning with “PRO0483 protein”
Having a definition beginning with “MNC”
Having a definition beginning with “MOST-1”
Having a definition beginning with “similar to”
Having a definition including “TPR gene on Y”
Having a definition beginning with “HSPC”
Having a definition beginning with “CGI-”
ReFSeq sequence composed of self only (information referenced from LL_tmpl)

The annotation information obtained by this analysis is shown in Tables 3-1 to 3-8.

TABLE 3-1

| SEQ ID | | RefSeq(BLASTP) | | SwissProt(BLASTP) | | |
|---|---|---|---|---|---|---|
| NO: | FLJ No. | RS Definition | Acc. No. | SP Definition | Acc. No. | KW |
| 1 | FLJ21182 | calponin 2 isoform a | NP_004359.1 | Calponin-2 (Calponin H2, smooth | Q99439 | Actin-binding; Calmodulin- |
| | | [Homo sapiens] | | muscle)(Neutral calponin) | | binding; Direct protein |
| | | | | | | sequencing; Multigene family; |
| | | | | | | Repeat. |
| 2 | FLJ38597 | smoothelin isoform b | NP_599031.1 | Smoothelin | P53814 | Alternative splicing; |
| | | [Homo sapiens] | | | | Phosphorylation; Structural |
| | | | | | | protein. |
| 3 | FLJ13700 | spectrin, beta, non- | NP_003119.1 | Spectrin beta chain, brain 1 | Q01082 | 3D-structure; Actin capping; |
| | | erythrocytic 1 | | (Spectrin, non-erythroid beta | | Actin-binding; Alternative |
| | | isoform 1 [Homo | | chain 1) (Beta-II spectrin) | | splicing; Calmodulin-binding; |
| | | sapiens] | | (Fodrinbeta chain) | | Cytoskeleton; Membrane; |
| | | | | | | Phosphorylation; Repeat. |
| 4 | FLJ50683 | plastin 3 [Homo | NP_005023.2 | T-plastin (Plastin-3) | P13797 | 3D-structure; Actin-binding; |
| | | sapiens] | | | | Calcium; Direct protein |
| | | | | | | sequencing; Phosphorylation; |
| | | | | | | Repeat. |
| 5 | FLJ50199 | Rac/Cdc42 guanine | NP_004831.1 | Rho guanine nucleotide exchange | Q15052 | 3D-structure; Alternative |
| | | nucleotide exchange | | factor 6(Rac/Cdc42 guanine | | splicing; Guanine-nucleotide |
| | | factor 6 [Homo | | nucleotide exchange factor 6) | | releasing factor; |
| | | sapiens] | | (PAK-interacting exchange factor | | Phosphorylation; SH3 domain. |
| | | | | alpha) (Alpha-Pix)(COOL-2) | | |
| 6 | FLJ26440 | chromosome 6 open | NP_981932.1 | Putative NADH dehydrogenase/ | O26223 | Complete proteome; Flavoprotein; |
| | | reading frame 71 | | NAD(P)H nitroreductase | | FMN; Hypothetical protein; NAD; |
| | | [Homo sapiens] | | (EC 1.—.—.—) | | NADP; Oxidoreductase. |
| 7 | FLJ21647 | RAN binding protein | NP_015561.1 | Ran-binding protein 3 (RanBP3) | Q9H6Z4 | Alternative splicing; Nuclear |
| | | 3 isoform RANBP3-d | | | | protein; Protein transport; |
| | | [Homo sapiens] | | | | Transport. |

TABLE 3-2

| | | | | | | |
|---|---|---|---|---|---|---|
| 8 | FLJ26620 | gelsolin-like capping | NP_001738.2 | Macrophage capping protein | P40121 | 3D-structure; Actin capping; |
| | | protein [Homo sapiens] | | (Actin-regulatoryprotein | | Actin-binding; Direct protein |
| | | | | CAP-G) | | sequencing; Nuclear protein; |
| | | | | | | Repeat. |
| 9 | FLJ43792 | guanylate cyclase | NP_000400.2 | Guanylate cyclase-activating | P43080 | Calcium; Disease mutation; |
| | | activator 1A (retina) | | protein 1 (GCAP1) (Guanylate | | Lipoprotein; Myristate; Repeat; |
| | | [Homo sapiens] | | cyclase activator 1A) | | Sensory transduction; Vision. |
| 10 | FLJ38127 | | | | | |
| 11 | FLJ35050 | pyruvate kinase 3 | NP_872271.1 | Pyruvate kinase, isozyme M1 | P11979 | 3D-structure; Acetylation; |
| | | isoform 2 [Homo | | (EC 2.7.1.40)(Pyruvate | | Alternative splicing; Direct |
| | | sapiens] | | kinase muscle isozyme) | | protein sequencing; Glycolysis; |
| | | | | | | Kinase; Magnesium; Metal-binding; |
| | | | | | | Multigene family; Transferase. |
| 12 | FLJ27298 | ras homolog gene | NP_001655.1 | Transforming protein RhoA | P61586 | 3D-structure; ADP-ribosylation; |
| | | family, member A | | (H12) | | Cytoskeleton; Direct protein |
| | | [Homo sapiens] | | | | sequencing; GTP-binding; |
| | | | | | | Lipoprotein; Magnesium; Membrane; |
| | | | | | | Methylation; Nucleotide-binding; |
| | | | | | | Prenylation; Prato-oncogene. |
| 13 | FLJ26262 | chloride intracellular | NP_001279.2 | Chloride intracellular | O00299 | 3D-structure; Acetylation; |
| | | channel 1 [Homo | | channel protein 1(Nuclear | | Chloride; Chloride channel; |
| | | sapiens] | | chloride ion channel 27) | | Direct protein sequencing; Ion |
| | | | | (NCC27) (p64 CLCP)(Chloride | | transport; Ionic channel; Nuclear |
| | | | | channel ABP) (Regulatory | | protein; Transport; Voltage- |
| | | | | nuclear chloride ion channel | | gated channel. |
| | | | | protein) (hRNCC) | | |
| 14 | FLJ90682 | chloride intracellular | NP_058625.1 | Chloride intracellular | Q9EPT8 | Chloride; Chloride channel; Ion |
| | | channel 5 [Homo | | channel protein 5 | | transport; Ionic channel; |
| | | sapiens] | | | | Transport; Voltage-gated channel. |
| 15 | FLJ22923 | target of myb1 | NP_005479.1 | Target of Myb protein 1 | O60784 | 3D-structure; Membrane; Protein |
| | | [Homo sapiens] | | | | transport; Transport. |

TABLE 3-3

| | | | | | | |
|---|---|---|---|---|---|---|
| 16 | FLJ22871 | polymerase (RNA) III (DNA | NP_612211.1 | DNA-dependent RNA polymerase | Q9Y535 | Alternative splicing; DNA- |
| | | dependent) polypeptide H | | III subunit 22.9 kDa polypeptide | | dependent RNA polymerase; |
| | | (22.9 kD) isoform a [Homo | | (EC 2.7.7.6) (RPC8) | | Nuclear protein; |
| | | sapiens] | | | | Nucleotidyltransferase; |
| | | | | | | Transcription; Transferase. |
| 17 | FLJ20398 | ubiquitin-like 4 [Homo | NP_055050.1 | Ubiquitin-like protein 4 | P11441 | |
| | | sapiens] | | (Ubiquitin-likeprotein GDX) | | |
| 18 | FLJ35377 | ubiquitin-binding protein | NP_613055.1 | | | |
| | | homolog [Mus musculus] | | | | |
| 19 | FLJ42145 | ubiquitin-binding protein | NP_613055.1 | | | |
| | | homolog [Mus musculus] | | | | |
| 20 | FLJ26144 | glucosamine-6-phosphate | NP_612208.1 | Glucosamine-6-phosphate | Q64422 | Carbohydrate metabolism; |
| | | deaminase 2 [Homo sapiens] | | isomerase (EG3.5.99.6) | | Hydrolase. |
| | | | | (Glucosamine-6-phosphate | | |
| | | | | deaminase) (GNPDA)(GlcN6P | | |
| | | | | deaminase) (Oscillin) | | |
| 21 | FLJ26374 | glucose phosphate | NP_000166.2 | Glucose-6-phosphate isomerase | P06744 | 3D-structure; Acetylation; |
| | | isomerase [Homo sapiens] | | (EC 5.3.1.9)(GPI) | | Cytokine; Direct protein |
| | | | | (Phosphoglucose isomerase) | | sequencing; Disease mutation; |
| | | | | (PGI) (Phosphohexose isomerase) | | Gluconeogenesis; Glycolysis; |
| | | | | (PHI) (Neuroleukin) (NLK) | | Growth factor; Isomerase; |
| | | | | (Sperm antigen 36)(SA-36) | | Polymorphism. |
| 22 | FLJ26371 | lactate dehydrogenase B | NP_002291.1 | L-lactate dehydrogenase B chain | P07195 | 3D-structure; Acetylation; |
| | | [Homo sapiens] | | (EC 1.1.1.27)(LDH-B) (LDH | | Direct protein sequencing; |
| | | | | heart subunit) (LDH-H) | | Disease mutation; Glycolysis; |
| | | | | | | Multigene family; NAD; |
| | | | | | | Oxidoreductase. |

TABLE 3-4

| | | | | | | |
|---|---|---|---|---|---|---|
| 23 | FLJ45688 | protein phosphatase | NP_817092.1 | Protein phosphatase 2C gamma isoform | O15355 | Hydrolase; Magnesium; |
| | | 1G [Homo sapiens] | | (EC3.1.3.16) (PP2C-gamma) (Protein | | Manganese; Metal-binding; |
| | | | | phosphatase magnesium-dependent 1 | | Multigene family; |
| | | | | gamma) (Protein phosphatase 1C) | | Protein phosphatase. |
| 24 | FLJ38620 | proline arginine rich | NP_659190.2 | Inner centromere protein | Q9NQS7 | Cell cycle; Cell division; |
| | | coiled coil 1 [Mus | | | | Centromere; Coiled coil; |
| | | musculus] | | | | Microtubule; Mitosis; Nuclear |
| | | | | | | protein. |
| 25 | FLJ26267 | protein-L-isoaspartate | NP_005380.1 | Protein-L-isoaspartate(D-aspartate)O- | P22061 | 3D-structure; Acetylation; |
| | | (D-aspartate) O- | | methyltransferase (EC 2.1.1.77)(Protein- | | Alternative splicing; Direct |
| | | methyltransferase | | beta-aspartate methyltransferase) | | protein sequencing; |
| | | [Homo sapiens] | | (PIMT)(Protein L-isoaspartyl/D-aspartyl | | Methyltransferase; |
| | | | | methyltransferase)(L-isoaspartyl protein | | Polymorphism; Transferase. |
| | | | | carboxyl methyltransferase) | | |
| 26 | FLJ26062 | glyoxalase I [Homo | NP_006699.1 | Lactoylglutathione lyase (EC 4.4.1.5) | Q04760 | 3D-structure; Lyase; Metal- |
| | | sapiens] | | (Methylglyoxalase) (Aldoketomutase) | | binding; Polymorphism; Zinc. |
| | | | | (Glyoxalase I) (GlxI) (Ketone-aldehyde | | |
| | | | | mutase) (S-D-lactoylglutathionemethyl- | | |
| | | | | glyoxal lyase) | | |
| 27 | FLJ22936 | septin 6 isoform D | NP_665801.1 | Septin-6 | Q14141 | Acetylation; Alternative |
| | | [Homo sapiens] | | | | splicing; Cell cycle; Cell |
| | | | | | | division; Coiled coil; Direct |
| | | | | | | protein sequencing; GTP- |
| | | | | | | binding; Nucleotide-binding. |

TABLE 3-5

| | | | | | | |
|---|---|---|---|---|---|---|
| 28 | FLJ43223 | tyrosyl-tRNA synthetase | NP_003671.1 | Tyrosyl-tRNA synthetase, cytoplasmic | P54577 | 3D-structure; Acetylation; |
| | | [Homo sapiens] | | (EC6.1.1.1) (Tyrosyl-tRNA ligase) | | Aminoacyl-tRNA synthetase; |
| | | | | (TyrRS) | | ATP-binding; Direct protein |
| | | | | | | sequencing; Ligase; Nucleotide- |
| | | | | | | binding; Protein biosynthesis; |
| | | | | | | RNA-binding; tRNA-binding. |
| 29 | FLJ26102 | solute carrier family 31 | NP_001850.1 | activating transcription factor 7 | O15431 | Copper; Copper transport; Ion |
| | | (copper transporters), | | interacting protein 2 [Homo sapiens] | | transport; Transmembrane; |
| | | member 1 [Homo | | | | Transport. |
| | | sapiens] | | | | |
| 30 | FLJ25218 | | | | | |
| 31 | FLJ45675 | | | Protein C17 orf39 | Q8IVV7 | |
| 32 | FLJ25918 | | | | | |
| 33 | FLJ46709 | transmembrane protein | NP_055622.3 | Transmembrane protein 24 (DLNB23 | O14523 | Transmembrane. |
| | | 24 [Homo sapiens] | | protein) | | |
| 35 | FLJ40377 | Akt-phosphorylation | NP_789811.2 | | | |
| | | enhancer [Mus | | | | |
| | | musculus] | | | | |
| 36 | FLJ25845 | armadillo repeat | NP_775104.1 | Serine/threonine-protein kinase | Q05609 | ATP-binding; Ethylene signaling |
| | | containing 3 | | CTR1 (EC2.7.1.37) | | pathway; Kinase; Nucleotide- |
| | | [Homo sapiens] | | | | binding; Serine/threonine-protein |
| | | | | | | kinase; Transferase. |
| 37 | FLJ23662 | DIPB protein [Homo | NP_060053.2 | Tripartite motif protein 44 (DIPB | Q96DX7 | Coiled coil; Metal-binding; Zinc; |
| | | sapiens] | | protein) | | Zinc-finger. |
| 38 | FLJ12668 | activating transcription | NP_079273.2 | | | |
| | | factor 7 interacting | | | | |
| | | protein 2 [Homo sapiens] | | | | |
| 39 | FLJ90085 | Ran-binding protein 10 | NP_665823.2 | Ran binding protein 9 (RanBP9) | P69566 | Nuclear protein; Phosphorylation; |
| | | [Mus musculus] | | (Ran-binding protein M) (RanBPM) | | Ubl conjugation. |
| | | | | (B cell antigen receptor Ig beta | | |
| | | | | associated protein 1) (IBAP-1) | | |

TABLE 3-6

| | | | | | | |
|---|---|---|---|---|---|---|
| 40 | FLJ90364 | nudix -type motif 9 | NP_932156.1 | ADP-ribose pyrophosphatase, mitochondrial | Q9BW91 | 3D-structure; Alternative |
| | | isoform a [Homo | | precursor (EC 3.6.1.13) (ADP-ribose | | splicing; Hydrolase; |
| | | sapiens] | | diphosphatase)(Adenosine diphosphoribose | | Magnesium; Manganese; |
| | | | | pyrophosphatase) (ADPR-PPase)(ADP-ribose | | Mitochondrion; Transit |
| | | | | phosphohydrase) (Nucleoside diphosphate- | | peptide. |
| | | | | linked moiety X motif 9) (Nudix motif 9) | | |
| 41 | FLJ90401 | ELOVL family member | NP_076995.1 | Elongated protein 3 of very long chain | Q9HB03 | Endoplasmic reticulum; |
| | | 6, elongation of | | fatty acids (30 kDa of Cold inducible | | Fatty acid biosynthesis; |
| | | long chain fatty | | glycoprotein) | | Lipid synthesis; |
| | | acids (FEN1/Elo2, | | | | Transmembrane. |
| | | SUR4/Elo3-like, yeast) | | | | |
| | | [Homo sapiens] | | | | |
| 42 | FLJ25526 | brain-specific protein | NP_008961.1 | Tubulin polymerization-promoting | O94811 | Phosphorylation. |
| | | p25 alpha [Homo | | protein(TPPP) (25 kDa brain-specific | | |
| | | sapiens] | | protein) (p25-alpha) (p24)(p25) | | |
| 43 | FLJ46896 | SH3 multiple domains | NP_032044.1 | Neutrophil cytosol factor 1 (NCF-1) | P14598 | 3D-structure; Chronic |
| | | 1 [Mus musculus] | | (Neutrophil NADPH oxidase factor 1) | | granulomatous disease; |
| | | | | (47 kDa neutrophiloxidase factor) | | Disease mutation; |
| | | | | (p47-phox) (NCF-47K) (47 kDa autosomal | | Polymorphism; Repeat; |
| | | | | chronic granulomatous disease protein) | | SH3 domain. |
| | | | | (NOXO2) | | |
| 44 | FLJ46856 | aortic preferentially | NP_005867.2 | Aortic preferentially expressed protein | Q15772 | Immunoglobulin domain; |
| | | expressed gene 1 [Homo | | 1(APEG-1) | | Nuclear protein. |
| | | sapiens] | | | | |
| 45 | FLJ90345 | sine oculis homeobox | NP_787071.2 | Homeobox protein SIX5 (DM locus- | Q8N196 | Activator; Alternative |
| | | homolog 5 [Homo | | associated homeodomain protein) | | splicing; Developmental |
| | | sapiens] | | | | protein; DNA-binding; |
| | | | | | | Homeobox; Nuclear protein; |
| | | | | | | Transcription; |
| | | | | | | Transcription regulation. |

TABLE 3-7

| | | | | | | |
|---|---|---|---|---|---|---|
| 46 | FLJ26550 | transaldolase 1 [Homo | NP_006746.1 | Transaldolase (EC 2.2.1.2) | P37837 | 3D-structure; Disease |
| | | sapiens] | | | | mutation; Pentose shunt; |
| | | | | | | Transferase. |
| 47 | FLJ90015 | Mof4 family associated | NP_150638.1 | | | |
| | | protein 1 [Homo | | | | |
| | | sapiens] | | | | |
| 48 | FLJ39454 | von Willebrand factor | NP_954572.1 | Protein KIAA1510 precursor | Q9P218 | Alternative splicing; |
| | | A domain-associated | | | | Collagen; Glycoprotein; |
| | | protein isoform 2 | | | | Repeat; Signal. |
| | | [Homo sapiens] | | | | |
| 49 | FLJ45115 | E1A binding protein | NP_056224.2 | E1A binding protein p400 | Q96L91 | Alternative splicing; |
| | | p400 [Homo sapiens] | | (EC 3.6.1.—) (p400 kDaSWI2/ | | ATP-binding; Chromatin |
| | | | | SNF2-associated protein) | | regulator; DNA-binding; |
| | | | | (Domino homolog) (hDomino) | | Helicase; Hydrolase; |
| | | | | (CAG repeat protein 32) | | Nuclear protein; |
| | | | | (Trinucleotide repeat- | | Nucleotide-binding; |
| | | | | containing gene 12 protein) | | Phosphorylation. |
| 50 | FLJ90066 | BM88 antigen [Homo | NP_057648.2 | BM88 antigen | Q8N111 | Antigen; Transmembrane. |
| | | sapiens] | | | | |
| 51 | FLJ37995 | carbonic anhydrase | NP_940986.1 | Carbonic anhydrase 13 (EC | Q8N1Q1 | Lyase; Metal-binding Zinc. |
| | | XIII [Homo sapiens] | | 4.2.1.1) (Carbonic anhydrase | | |
| | | | | XIII) (Carbonate dehydratase | | |
| | | | | XIII) (CA-XIII) | | |
| 52 | FLJ26058 | eukaryotic translation | NP_001395.1 | Elongation factor 1-gamma | P26641 | 3D-structure; Acetylation; |
| | | elongation factor 1 | | (EF-1-gamma) (eEF-1Bgamma) | | Direct protein sequencing; |
| | | gamma [Homo sapiens] | | | | Elongation factor; Protein |
| | | | | | | biosynthesis. |
| 53 | FLJ46369 | proteoglycan 4 | NP_005798.2 | Cytadherence high molecular | Q50365 | Complete proteome; |
| | | [Homo sapiens] | | weight protein 1(Cytadherence | | Cytadherence; Direct |
| | | | | accessory protein 1) | | protein sequencing; |
| | | | | | | Structural protein. |
| 54 | FLJ16517 | lin-28 homolog | NP_078950.1 | Y-box binding protein 2-A | P21574 | Direct protein sequencing; |
| | | [Homo sapiens] | | (Cytoplasmic RNA-binding | | DNA-binding; Nuclear protein; |
| | | | | protein p56) (mRNP4) | | Phosphorylation; RNA-binding; |
| | | | | | | Transcription; Transcription |
| | | | | | | regulation. |

TABLE 3-8

| | | | | | | |
|---|---|---|---|---|---|---|
| 55 | FLJ26591 | peptidylprolyl | NP_066953.1 | Peptidyl-prolyl cis-trans isomerase | P62941 | Cyclosporin; Isomerase; |
| | | isomerase A isoform | | A (EC5.2.1.8) (PPIase) (Rotamase) | | Multigene family; |
| | | 1 [Homo sapiens] | | (Cyclophilin A)(Cyclosporin A- | | Rotamase. |
| | | | | binding protein) | | |
| 56 | FLJ26596 | H2B histone family, | NP_003511.1 | Histone H2B.d (H2B/d) | Q99877 | Chromosomal protein; |
| | | member D [Homo | | | | DNA-binding; Multigene |
| | | sapiens] | | | | family; Nuclear protein; |
| | | | | | | Nucleosome core. |
| 57 | FLJ90480 | zinc finger, CCCH- | NP_852149.1 | Zinc finger CCCH-type with G patch | Q8N5A5 | Alternative splicing; |
| | | type with G patch | | domainprotein (Zinc finger CCCH- | | Metal-binding; Zinc; |
| | | domain isoform b | | type domain containing protein 9) | | Zinc-finger. |
| | | [Homo sapiens] | | | | |
| 58 | FLJ43067 | phosphoglycerate | NP_002620.1 | Phosphoglycerate mutase 1 (EC | P18669 | 3D-structure; Acetylation; |
| | | mutase 1 (brain) | | 5.4.2.1) (EC5.4.2.4) (EC 3.1.3.13) | | Direct protein sequencing; |
| | | [Homo sapiens] | | (Phosphoglycerate mutase isozymeB) | | Glycolysis; Hydrolase; |
| | | | | (PGAM-B) (BPG-dependent PGAM 1) | | Isomerase. |
| 59 | FLJ25460 | | | | | |
| 60 | FLJ26806 | | | | | |
| 61 | FLJ43911 | retrotransposon- | NP_908998.1 | Midasin (MIDAS-containing protein) | Q9NU22 | ATP-binding; Chaperone; |
| | | like 1 [Mus | | | | Nuclear protein; |
| | | musculus] | | | | Nucleotide-binding; |
| | | | | | | Phosphorylation; Repeat. |
| 62 | FLJ44715 | | | | | |
| 63 | FLJ90031 | polymerase I and | NP_036364.2 | Polymerase I and transcript release | Q6NZI2 | Acetylation; Alternative |
| | | transcript release | | factor(PTRF protein) | | splicing; Direct protein |
| | | factor [Homo | | | | sequencing; Membrane; |
| | | sapiens] | | | | Nuclear protein; |
| | | | | | | Phosphorylation; RNA- |
| | | | | | | binding; rRNA-binding; |
| | | | | | | Transcription; Transcription |
| | | | | | | regulation; Transcription |
| | | | | | | termination. |

### Amino Acid Analysis 2

### Motif Analysis by Pfam

The calculation program used was hmmpfam (v2.3.2). The target databases used were Pfam DB entry: 7973 families (Pfam18.0, Pfam_ls). (July 2005). The cutoff value was established at 1E-10. The annotation information obtained by this analysis is shown by Tables 4-1 to 4-3.

TABLE 4-1

| SEQ | | | | |
|---|---|---|---|---|
| ID | FLJ# for | | | |
| NO: | reference | pfamID | Pfam Name | Pfam Description |
| 1 | FLJ21182 | PF00307.18¥PF00402.7 | CH¥Calponin | Calponin homology (CH) |
| | | | | domain¥Calponin family repeat |
| 2 | FLJ38597 | PF00307.18 | CH | Calponin homology (CH) domain |
| 3 | FLJ13700 | PF00169.16 | PH | PH domain |
| 4 | FLJ50683 | PF00307.18¥PF00307.18¥PF00307.18¥PF00307.18 | CH¥CH¥CH¥CH | Calponin homology (CH) |
| | | | | domain¥Calponin homology (CH) |
| | | | | domain¥Calponin homology (CH) |
| | | | | domain¥Calponin homology (CH) |
| | | | | domain |
| 5 | FLJ50199 | PF00018.16¥PF07653.5¥PF00621.9 | SH3_1¥SH3_2¥RhoGEF | SH3 domain¥Variant SH3 |
| | | | | domain¥RhoGEF domain |
| 6 | FLJ26440 | | | |
| 7 | FLJ21647 | | | |
| 8 | FLJ26620 | PF00626.11¥PF00626.11¥PF00626.11 | Gelsolin¥Gelsolin¥Gelsolin | Gelsolin repeat¥Gelsolin |
| | | | | repeat¥Gelsolin repeat |
| 9 | FLJ43792 | | | |
| 10 | FLJ38127 | | | |
| 11 | FLJ35050 | PF00224.10¥PF02887.5 | PK¥PK_C | Pyruvate kinase, barrel |
| | | | | domain¥Pyruvate kinase, |
| | | | | alpha/beta domain |
| 12 | FLJ27298 | PF00071.11 | Ras | Ras family |
| 13 | FLJ26262 | | | |
| 14 | FLJ90682 | | | |
| 15 | FLJ22923 | PF00790.8¥PF03127.4 | VHS¥GAT | VHS domain¥GAT domain |
| 16 | FLJ22871 | PF03876.5¥PF08292.1 | RNA_pol_Rpb7_N¥RNA_pol_Rbc25 | RNA polymerase Rpb7, N-terminal |
| | | | | domain¥RNA polymerase III |
| | | | | subunit Rpc25 |

TABLE 4-2

| | | | | |
|---|---|---|---|---|
| 17 | FLJ20398 | PF00240.12 | ubiquitin | Ubiquitin family |
| 18 | FLJ35377 | PF00240.12 | ubiquitin | Ubiquitin family |
| 19 | FLJ42145 | | | |
| 20 | FLJ26144 | PF01182.10 | Glucosamine_iso | Glucosamine-6-phosphate isomerases/6- |
| | | | | phosphogluconolactonase |
| 21 | FLJ26374 | PF00342.8 | PGI | Phosphoglucose isomerase |
| 22 | FLJ26371 | PF00056.11¥PF02866.6 | Ldh_1_N¥Ldh_1_C | lactate/malate dehydrogenase, NAD binding |
| | | | | domain¥lactate/malate dehydrogenase, alpha/beta C-terminal |
| | | | | domain |
| 23 | FLJ45688 | PF00481.10 | PP2C | Protein phosphatase 2C |
| 24 | FLJ38620 | PF05672.1 | E-MAP-115 | E-MAP-115 family |
| 25 | FLJ26267 | PF01135.8 | PCMT | Protein-L-isoaspartate(D-aspartate) O-methyltransferase |
| | | | | (PCMT) |
| 26 | FLJ26062 | PF00903.14 | Glyoxalase | Glyoxalase/Bleomycin resistance protein/Dioxygenase superfamily |
| 27 | FLJ22936 | PF00735.8 | GTP_CDC | Cell division protein |
| 28 | FLJ43223 | PF00579.13¥PF01588.8 | tRNA-synt_1b¥tRNA_bind | tRNA synthetases class I (W and Y)¥Putative tRNA binding |
| | | | | domain |
| 29 | FLJ26102 | PF04145.5 | Ctr | Ctr copper transporter family |
| 30 | FLJ25218 | | | |
| 31 | FLJ45675 | | | |
| 32 | FLJ25918 | PF05368.2 | NmrA | NmrA-like family |
| 33 | FLJ46709 | | | |
| 35 | FLJ40377 | | | |
| 36 | FLJ25845 | | | |
| 37 | FLJ23662 | PF00643.13 | zf-B_box | B-box zinc finger |
| 38 | FLJ12668 | | | |

TABLE 4-3

| | | | | |
|---|---|---|---|---|
| 39 | FLJ90085 | | | |
| 40 | FLJ90364 | | | |
| 41 | FLJ90401 | | | |
| 42 | FLJ25526 | PF05517.2 | p25-alpha | p25-alpha |
| 43 | FLJ46896 | PF00787.12 | PX | PX domain |
| 44 | FLJ46856 | PF07679.3 | I-set | Immunoglobulin I-set domain |
| 45 | FLJ90345 | | | |
| 46 | FLJ26550 | PF00923.8 | Transaldolase | Transaldolase |
| 47 | FLJ90015 | | | |
| 48 | FLJ39454 | PF00041.10 | fn3 | Fibronectin type III domain |
| 49 | FLJ45115 | | | |
| 50 | FLJ90066 | | | |
| 51 | FLJ37995 | PF00194.10 | Carb_anhydrase | Eukaryotic-type carbonic anhydrase |
| 52 | FLJ26058 | PF02798.8¥PF00043.13¥PF00647.8 | GST_N¥GST_C¥EF1G | Glutathione S-transferase, N-terminal domain¥Glutathione S- |
| | | | | transferase, C-terminal domain¥Elongation factor 1 gamma, |
| | | | | conserved domain |
| 53 | FLJ46369 | | | |
| 54 | FLJ16517 | PF00313.11 | CSD | ‘Cold-shock’ DNA-binding domain |
| 55 | FLJ26591 | PF00160.10 | Pro_isomerase | Cyclophilin type peptidyl-prolyl cis-trans isomerase |
| 56 | FLJ26596 | PF00125.12 | Histone | Core histone H2A/H2B/H3/H4 |
| 57 | FLJ90480 | | | |
| 58 | FLJ43067 | PF00300.11 | PGAM | Phosphoglycerate mutase family |
| 59 | FLJ25460 | | | |
| 60 | FLJ26806 | | | |
| 61 | FLJ43911 | | | |
| 62 | FLJ44715 | | | |
| 63 | FLJ90031 | | | |

### Amino Acid Analysis 3

### Prediction of Secretory Signal Sequences by Signal IP

The calculation program used was PSORT II, SignalP ver3.0 (May 18, 2004), and SOSui ver1.5.

### Amino Acid Analysis 4

### Functional Categorization by GeneOntology

Performed per the procedures described below.
1) Extract results having E-values that meet the following conditions from among the results of homology analysis using BLASTP (RefSeq and SwissProt with filter) that produced three higher BLAST results (six in total).
Condition 1: Use all results having E-values of not more than 1E-50.
Condition 2: Do not use results having E-values of not less than 1E-10.
Condition 3: Use results having E-values exceeding 1E-50, provided that the difference in E-value from Top Hit is within 1E+20.
Condition 4: If the E-value of Top Hit is 0, use results having E-values of not more than 1E-50.
2) Search GO by the keywords of SwissProt using spkw2go.
3) Search xref.goa by accession numbers of SwissProt to acquire Refseq IDs, further acquire LOCUS IDs by the Refseq IDs using LL_tmpl, and acquire GO terms by the LOCUS IDs using loc2go.
4) Acquire LOCUS IDs by accession numbers of Refseq using LL_tmpl, and acquire GO terms by the LOCUS IDs using loc2go.
5) Acquire information on higher categories for each GO term acquired, with reference to the Molecular Function text file, Biological Process text file, and Cellular Component text file.
6) Remove overlapping information from the GO term information acquired in 1)-5) above, and make an output.

The annotation information obtained by this analysis is shown in Tables 5-1 and 5-4.

TABLE 5-1

| SEQ | | |
|---|---|---|
| ID | | |
| NO: | FLJ No. | GO No.(term) |
| 1 | FLJ21182 | GO:0003779¥MF\|actin binding; GO:0005516¥MF\|calmodulin binding; GO:0006939¥BP\|smooth muscle contraction; |
| | | GO:0007010¥BP\|cytoskeleton organization and biogenesis; GO:0005856¥CC\|cytoskeleton; GO:0005911¥CC\|intercellular |
| | | junction |
| 2 | FLJ38597 | GO:0003779¥MF\|actin binding; GO:0008307¥MF\|structural constituent of muscle; GO:0006939¥BP\|smooth muscle contraction; |
| | | GO:0007517¥BP\|muscle development; GO:0015629¥CC\|actin cytoskeleton |
| 3 | FLJ13700 | GO:0003779¥MF\|actin binding; GO:0005200¥MF\|structural constituent of cytoskeleton; GO:0005515¥MF\|protein binding; |
| | | GO:0005516¥MF\|calmodulin binding; GO:0007182¥BP\|common-partner SMAD protein phosphorylation; |
| | | GO:0007184¥BP\|SMAD protein nuclear translocation; GO:0005634¥CC\|nucleus; GO:0005856¥CC\|cytoskeleton; |
| | | GO:0005886¥CC\|plasma membrane; GO:0008091¥CC\|spectrin; GO:0016020¥CC\|membrane |
| 4 | FLJ50683 | GO:0003779¥MF\|actin binding; GO:0005509¥MF\|calcium ion binding; GO:0000004¥BP\|biological process unknown; |
| | | GO:0005829¥CC\|cytosol; GO:0015629¥CC\|actin cytoskeleton |
| 5 | FLJ50199 | GO:0005089¥MF\|Rho guanyl-nucteotide exchange factor activity; GO:0005096¥MF\|GTPase activator activity; |
| | | GO:0005554¥MF\|molecular function unknown; GO:0000004¥BP\|biological process unknown; GO:0006915¥BP\|apoptosis; |
| | | GO:0007254¥BP\|JNK cascade; GO:0005622¥CC\|intracellular; GO:0008372¥CC\|cellular component unknown |
| 6 | FLJ26440 | GO:0016491¥MF\|oxidoreductase activity; GO:0006118¥BP\|electron transport |
| 7 | FLJ21647 | GO:0008536¥MF\|Ran GTPase binding; GO:0006810¥BP\|transport; GO:0007264¥BP\|small GTPase mediated signal transduction; |
| | | GO:0015031¥BP\|protein transport; GO:0005634¥CC\|nucleus; GO:0005643¥CC\|nuclear pore |
| 8 | FLJ26620 | GO:0003779¥MF\|actin binding; GO:0006461¥BP\|protein complex assembly; GO:0009613¥BP\|response to pest, pathogen or |
| | | parasite; GO:0030031¥BP\|cell projection biogenesis; GO:0051016¥BP\|barbed-end actin filament capping; |
| | | GO:0005634¥CC\|nucleus; GO:0005856¥CC\|cytoskeleton; GO:0008290¥CC\|F-actin capping protein complex |
| 9 | FLJ43792 | GO:0005509¥MF\|calcium ion binding; GO:0008048¥MF\|calcium sensitive guanylate cyclase activator activity; |
| | | GO:0030249¥MF\|guanylate cyclase regulator activity; GO:0007165¥BP\|signal transduction; GO:0007600¥BP\|sensory perception; |
| | | GO:0007601¥BP\|visual perception; GO:0007602¥BP\|phototransduction; GO:0031282¥BP\|regulation of guanylate cyclase activity |
| 10 | FLJ38127 | |
| 11 | FLJ35050 | GO:0000287¥MF\|magnesium ion binding; GO:0004743¥MF\|pyruvate kinase activity; GO:0016301¥MF\|kinase activity; |
| | | GO:0016740¥MF\|transferase activity; GO:0006096¥BP\|glycolysis; GO:0005739¥CC\|mitochondrion; GO:0005829¥CC\|cytosol |
| 12 | FLJ27298 | GO:0000287¥MF\|magnesium ion binding; GO:0003924¥MF\|GTPase activity; GO:0004871¥MF\|signal transducer activity; |
| | | GO:0005525¥MF\|GTP binding; GO:0007155¥BP\|cell adhesion; GO:0007160¥BP\|cell-matrix adhesion; |
| | | GO:0007229¥BP\|integrin-mediated signaling pathway; GO:0007264¥BP\|small GTPase mediated signal transduction; |
| | | GO:0007266¥BP\|Rho protein signal transduction; GO:0007519¥BP\|myogenesis; GO:0015031¥BP\|protein transport; |
| | | GO:0030036¥BP\|actin cytoskeleton organization and biogenesis; GO:0030154¥BP\|cell differentiation; |
| | | GO:0042346¥BP\|positive regulation of NF-kappaB-nucleus import; GO:0042346¥BP positive regulation of NF-kappaB-nucleus |
| | | import; GO:0043123¥BP\|positive regulation of I-kappaB kinase/NF-kappaB cascade; GO:0043149¥BP\|stress fiber |
| | | formation; GO:0005829¥CC\|cytosol; GO:0005856¥CC\|cytoskeleton; GO:0016020¥CC\|membrane |
| 13 | FLJ26262 | GO:0005247¥MF\|voltage-gated chloride channel activity; GO:0005262¥MF\|calcium channel activity; |
| | | GO:0006811¥BP\|ion transport; GO:0006816¥BP\|calcium ion transport; GO:0006821¥BP\|chloride transport; |
| | | GO:0005624¥CC\|membrane fraction; GO:0005635¥CC\|nuclear membrane; GO:0016020¥CC\|membrane |
| 14 | FLJ90682 | GO:0005216¥MF\|ion channel activity; GO:0005244¥MF\|voltage-gated ion channel activity; GO:0005247¥MF\|voltage- |
| | | gated chloride channel activity; GO:0005254¥MF\|chloride channel activity; GO:0015108¥MF\|chloride transporter activity; |
| | | GO:0006810¥BP\|transport; GO:0006811¥BP\|ion transport; GO:0006821¥BP\|chloride transport; GO:0007565¥BP\|pregnancy; |
| | | GO:0005626¥CC\|insoluble fraction; GO:0005794¥CC\|Golgi apparatus; GO:0015629¥CC\|actin cytoskeleton; |
| | | GO:0016020¥CC\|membrane; |
| 15 | FLJ22923 | GO:0005515¥MF protein binding; GO:0008565¥MF\|protein transporter activity; GO:0006810¥BP\|transport; |
| | | GO:0006886¥BP\|intracellular protein transport; GO:0006891¥BP\|intra-Golgi transport; GO:0006897¥BP\|indocytosis; |
| | | GO:0015031¥BP\|protein transport; GO:0016197¥BP\|endosome transport; GO:0005764¥CC\|lysosome; |
| | | GO:0005768¥CC\|endosome; GO:0005769¥CC\|early endosome; GO:0005795¥CC\|Golgi stack; GO:0005829¥CC\|cytosol; |
| | | GO:0016020¥CC\|membrane; GO:0016020¥CC\|membrane |

TABLE 5-2

| | | |
|---|---|---|
| 16 | FLJ22871 | GO:0003676¥MF\|nucleic acid binding; GO:0003899¥MF\|DNA-dependent RNA polymerase activity; |
| | | GO:0005506¥MF\|iron ion binding; GO:0005515¥MF\|protein binding; GO:0016740¥MF\|transferase |
| | | activity; GO:0016779¥MF\|nucleotidyltransferase activity; GO:00006099¥BP\|tricarboxylic acid cycle; |
| | | GO:0006101¥BP\|citrate metabolism; GO:0006350¥BP\|transcription; GO:0006383¥BP\|transcription |
| | | from RNA polymerase III promoter; GO:0005634¥CC\|nucleus; GO:0005666¥CC\|DNA-dependent RNA polymerase |
| | | III complex; GO:0005739¥CC\|mitrochondrion; |
| 17 | FLJ20398 | GO:0008639¥MF\|small protein conjugating enzyme activity; GO:0006464¥BP\|protein modification |
| 18 | FLJ35377 | |
| 19 | FLJ42145 | |
| 20 | FLJ26144 | GO:0004342¥MF\|glucosamine-6-phosphate deaminase activity; GO:0016787¥MF\|hydrolase activity; |
| | | GO:0016853¥MF\|isomerase activity; GO:0005975¥BP\|carbohydrate metabolism; GO:0006002¥BP\|fructose |
| | | 6-phosphate metabolism; GO:0006041¥BP\|glucosamine metabolism; GO:0006043¥BP\|glucosamine catabolism; |
| | | GO:0006044¥BP\|N-acetylglucosamine metabolism; GO:0006091¥BP\|generation of precursor metabolites and energy; |
| | | GO:0007338¥BP\|fertilization (metazoan animal); GO:0007340¥BP\|acrosome reaction; GO:0046370¥BP\|fructose |
| | | biosynthesis |
| 21 | FLJ26374 | GO:0004347¥MF\|glucose-6-phosphate isomerase activity; GO:0005125¥MF\|cytokine activity; |
| | | GO:0008083¥MF\|growth factor activity; GO:0016853¥MF\|isomerase activity; GO:0005975¥BP\|carbohydrate |
| | | metabolism; GO:0006094¥BP\|gluconeogenesis; GO:0006096¥BP\|glycolysis; GO:0006959¥BP\|humoral immune |
| | | response; GO:0007399¥BP\|neurogenesis; GO:0007599¥BP\|hemostasis |
| 22 | FLJ26371 | GO:0004457¥MF\|lactate dehydrogenase activity; GO:0004459¥MF\|L-lactate dehydrogenase activity; |
| | | GO:0005524¥MF\|ATP binding; GO:0016491¥MF\|oxidoreductase activity; GO:0006096¥BP\|glycolysis; |
| | | GO:0006100¥BP\|tricarboxylic acid cycle intermediate metabolism; GO:0019642¥BP\|anaerobic glycolysis; |
| | | GO:0005737¥CC\|cytoplasm |
| 23 | FLJ45688 | GO:0000287¥MF\|magnesium ion binding; GO:0003824¥MF\|catalytic activity; GO:0004721¥MF\|phosphoprotein |
| | | phosphatase activity; GO:0004722¥MF\|protein serine/threonine phosphatase activity; GO:0015071¥MF\|protein |
| | | phosphatase type 2C activity; GO:0016787¥MF\|hydrolase activity; GO:0030145¥MF\|manganese ion binding; |
| | | GO:0006470¥BP\|protein amino acid dephosphorylation; GO:0007049¥BP\|cell cycle; GO:0007050¥BP\|cell |
| | | cycle arrest; GO:0005634¥CC\|nucleus; GO:0008287¥CC\|protein serine/threonine phosphatase complex |
| 24 | FLJ38620 | GO:0005519¥MF\|cytoskeletal regulatory protein binding; GO:0007017¥BP\|microtubule-based process; |
| | | GO:0005875¥CC\|microtubule associated complex |
| 25 | FLJ26267 | GO:0004719¥MF\|protein-L-isoaspartate (D-aspartate) O-methyltransferase activity; GO:0008168¥MF\|methyltransferase |
| | | activity; GO:0008757¥MF\|S-adenosylmethionine-dependent methyltransferase activity; GO:0016740¥MF\|transferase |
| | | activity; GO:0006464¥BP\|protein modification; GO:0006479¥BP\|protein amino acid methylation; |
| | | GO:0005783¥CC\|endoplasmic reticulum |
| 26 | FLJ26062 | GO:0004462¥MF\|lactoylglutathione lyase activity; GO:0016829¥MF\|lyase activity; |
| | | GO:0005975¥BP\|carbohydrate metabolism |
| 27 | FLJ22936 | GO:0005515¥MF\|protein binding; GO:0005525¥MF\|GTP binding; GO:0000910¥BP\|cytokinesis; |
| | | GO:0007049¥BP\|cell cycle; GO:0008372¥CC\|cellular component unknown |
| 28 | FLJ43223 | GO:0000049¥MF\|tRNA binding; GO:0003723¥MF\|RNA binding; GO:0004812¥MF\|tRNA ligase activity; |
| | | GO:0004831¥MF\|tyrosine-tRNA ligase activity; GO:0004871¥MF signal transducer activity; GO:0005153¥MF\|interleukin- |
| | | 8 receptor binding; GO:0005524¥MF\|ATP binding; GO:0016874¥MF\|ligase activity; GO:0006412¥BP\|protein |
| | | biosynthesis; GO:0006418¥BP\|tRNA aminoacylation for protein translation; GO:0006437¥BP\|tyrosyl-tRNA aminoacylation; |
| | | GO:0006915¥BP\|apoptosis; GO:0006928¥BP\|cell motility; GO:0005615¥CC\|extracellular space; |
| | | GO:0005615¥CC\|extracellular space; GO:0005625¥CC\|soluble fraction; GO:0005737¥CC\|cytoplasm |
| 29 | FLJ26102 | GO:0005375¥MF\|copper ion transporter activity; GO:0005386¥MF\|carrier activity; GO:0006810¥BP\|transport; |
| | | GO:0006811¥BP\|ion transport; GO:0006825¥BP\|copper ion transport; GO:0005887¥CC\|integral to plasma membrane; |
| | | GO:0016021¥CC\|integral to membrane |
| 30 | FLJ25218 | |
| 31 | FLJ45675 | |
| 32 | FLJ25918 | |
| 33 | FLJ46709 | GO:0005554¥MF\|molecular function unknown; GO:0000004¥BP\|biological process unknown; GO:0016021¥CC\|integral |
| | | to membrane |

TABLE 5-3

| | | |
|---|---|---|
| 35 | FLJ40377 | |
| 36 | FLJ25845 | GO:0005488¥MF\|binding |
| 37 | FLJ23662 | GO:0008270¥MF\|zinc ion binding; GO:0005622¥CC\|intracellular |
| 38 | FLJ12668 | GO:0016021¥CC\|integral to membrane |
| 39 | FLJ90085 | GO:0016301¥MF\|kinase activity; GO:0004713¥MF\|protein-tyrosine kinase activity; GO:0004872¥MF\|receptor activity |
| 40 | FLJ90364 | GO:0000287¥MF\|magnesium ion binding; GO:0005227¥MF\|calcium activated cation channel activity; |
| | | GO:0016787¥MF\|hydrolase activity; GO:0019144¥MF\|ADP-sugar diphosphatase activity; GO:0030145¥MF\|manganese |
| | | ion binding; GO:0047631¥MF\|ADP-ribose diphosphatase activity; GO:0006812¥BP\|cation transport; |
| | | GO:0005622¥CC\|intracellular; GO:0005739¥CC\|mitochondrion |
| 41 | FLJ90401 | GO:0009922¥MF\|fatty acid elongase activity; GO:0016747¥MF\|transferase activity, transferring groups other than |
| | | amino-acyl groups; GO:0030497¥BP\|fatty acid elongation; GO:0016021¥CC\|integral to membrane; |
| | | GO:0030176¥CC\|integral to endoplasmic reticulum membrane |
| 42 | FLJ25526 | |
| 43 | FLJ46896 | GO:0008483¥MF\|transaminase activity; GO:0007242¥BP\|intracellular signaling cascade; |
| | | GO:0008152¥BP\|metabolism; GO:0015031¥BP\|protein transport |
| 44 | FLJ46856 | GO:0004674¥MF\|protein serine/threonine kinase activity; GO:0004713¥MF\|protein-tyrosine kinase activity; |
| | | GO:0005524¥MF\|ATP binding; GO:0016301¥MF\|kinase activity; GO:0016740¥MF\|transferase activity; |
| | | GO:0006468¥BP\|protein amino acid phosphorylation; GO:0007517¥BP\|muscle development; GO:0008285¥BP\|negative |
| | | regulation of cell proliferation; GO:0005634¥CC\|nucleus |
| 45 | FLJ90345 | GO:0003677¥MF\|DNA binding; GO:0003700¥MF\|transcription factor activity; GO:0006350¥BP\|transcription; |
| | | GO:0006355¥BP\|regulation of transcription, DNA-dependent; GO:0007275¥BP\|development; |
| | | GO:0045449¥BP\|regulation of transcription; GO:0005634¥CC\|nucleus; GO:0005667¥CC\|transcription factor complex |
| 46 | FLJ26550 | GO:0004801¥MF\|transaldolase activity; GO:0016740¥MF\|transferase activity; GO:0005975¥BP\|carbohydrate |
| | | metabolism; GO:0006098¥BP\|pentose-phosphate shunt; GO:0005737¥CC\|cytoplasm |
| 47 | FLJ90015 | GO:0005515¥MF\|protein binding; GO:0000004¥BP\|biological process unknown; GO:0008372¥CC\|cellular |
| | | component unknown |
| 48 | FLJ39454 | GO:0005554¥MF\|molecular function unknown; GO:0000004¥BP\|biological process unknown; |
| | | GO:0005576¥CC\|extracellular region; GO:0005615¥CC\|extracellular space |
| 49 | FLJ45115 | GO:0003677¥MF\|DNA binding; GO:0003705¥MF\|RNA polymerase II transcription factor activity, enhancer binding; |
| | | GO:0004386¥MF\|helicase activity; GO:0005524¥MF\|ATP binding; GO:0016787¥MF\|hydrolase activity; |
| | | GO:0030528¥MF\|transcription regulator activity; GO:0006355¥BP\|regulation of transcription, DNA-dependent; |
| | | GO:0006955¥BP\|immune response; GO:0016568¥BP\|chromatin modification; GO:0005634¥CC\|nucleus |
| 50 | FLJ90066 | GO:0005554¥MF\|molecular function unknown; GO:0000004¥BP\|biological process unknown; |
| | | GO:0016021¥CC\|integral to membrane |
| 51 | FLJ37995 | GO:0004089¥MF\|carbonate dehydratase activity; GO:0008270¥MF\|zinc ion binding; |
| | | GO:0016829¥MF\|lyase activity; GO:0006730¥BP\|one-carbon compound metabolism; GO:0005737¥CC\|cytoplasm |
| 52 | FLJ26058 | GO:0003746¥MF\|translation elongation factor activity; GO:0006412¥BP\|protein biosynthesis; |
| | | GO:0006414¥BP\|translational elongation; GO:0005622¥CC\|intracellular; GO:0005853¥CC\|eukaryotic |
| | | translation elongation factor 1 complex |
| 53 | FLJ46369 | GO:0004872¥MF\|receptor activity; GO:0004890¥MF\|GABA-A receptor activity; GO:0005198¥MF\|structural |
| | | molecule activity; GO:0005216¥MF\|ion channel activity; GO:0005230¥MF\|extracellular ligand-gated ion channel |
| | | activity; GO:0006810¥BP\|transport; GO:0006811¥BP\|ion transport; GO:0006821¥BP\|chloride transport; |
| | | GO:0007214¥BP\|gamma-aminobutyric acid signaling pathway; GO:0007268¥BP\|synaptic transmission; |
| | | GO:0045104¥BP\|intermediate filament cytoskeleton organization and biogenesis; GO:0005615¥CC\|extracellular |
| | | space; GO:0005739¥CC\|mitochondrion; GO:0005882¥CC\|intermediate filament; GO:0005882¥CC\|intermediate |
| | | filament; GO:0005883¥CC\|neurofilament; GO:0005887¥CC\|integral to plasma membrane; |
| | | GO:0016020¥CC\|membrane; GO:0016021¥CC\|integral to membrane |
| 54 | FLJ16517 | GO:0003677¥MF\|DNA binding; GO:0005554¥MF\|molecular function unknown; GO:0000004¥BP\|biological |
| | | process unknown; GO:0006355¥BP\|regulation of transcription, DNA-dependent; GO:0005737¥CC\|cytoplasm |

TABLE 5-4

| | | |
|---|---|---|
| 55 | FLJ26591 | GO:0003755¥MF\|peptidyl-prolyl cis-trans isomerase activity; GO:0016018¥MF\|cyclosporin A binding; |
| | | GO:0016853¥MF\|isomerase activity; GO:0046790¥MF\|virion binding; GO:0051082¥MF\|unfolded |
| | | protein binding; GO:0006457¥BP\|protein folding; GO:0045069¥BP\|regulation of viral genome replication; |
| | | GO:0005737¥CC\|cytoplasm; GO:0005829¥CC\|cytosol |
| 56 | FLJ26596 | GO:0003677¥MF\|DNA bindng; GO:0006334¥BP\|nucleosome assembly; GO:0007001¥BP\|chromosome |
| | | organization and biogenesis (Eukaryote); GO:000786¥CC\|nucleosome; GO:0005634¥CC\|nucleus; |
| | | GO:0005694¥CC\|chromosome |
| 57 | FLJ90480 | GO:0003676¥MF\|nucleic acid binding; GO:0005622¥CC\|intracellular |
| 58 | FLJ43067 | GO:0003824¥MF\|catalytic activity; GO:0004082¥MF\|bisphosphoglycerate mutase activity; |
| | | GO:0004083¥MF\|bisphosphoglycerate phosphatase activity; GO:0004619¥MF\|phosphoglycerate mutase |
| | | activity; GO:0016787¥MF\|hydrolase activity; GO:0016853¥MF\|isomerase activity; |
| | | GO:0016868¥MF\|intramolecular transferase activity, phosphotransferases; GO:0006096¥BP\|glycolysis; |
| | | GO:0008152¥BP\|metabolism; GO:0005829¥CC\|cytosol |
| 59 | FLJ25460 | |
| 60 | FLJ26806 | |
| 61 | FLJ43911 | |
| 62 | FLJ44715 | |
| 63 | FLJ90031 | GO:0003716¥MF\|RNA polymerase I transcription termination factor activity; GO:0003723¥MF\|RNA binding; |
| | | GO:0005515¥MF\|protein binding; GO:0019843¥MF\|rRNA binding; GO:0042134¥MF\|rRNA primary |
| | | transcript binding; GO:0006350¥BP\|transcription; GO:0006353¥BP\|transcription termination; |
| | | GO:0006355¥BP\|regulation of transcription, DNA-dependent; GO:0006361¥BP\|transcription initiation |
| | | from RNA polymerase I promoter; GO:0005634¥CC\|nucleus; GO:0016020¥CC\|membrane |

### Nucleic Acid Analysis 1

### Homology Analysis 1 by BLASTX

The calculation program used was blastall 2.2.6. The target database used was nr: 2972605 (2005.10.29). The cutoff value was established at 1.00E-05. The following data were processed by filtering:
Having a definition beginning with “ALU SUBFAMILY”
Having a definition including “Alu subfamily”
Having a definition beginning with “!!!! ALU SUBFAMILY”
Beginning with “Drosophila melanogaster genomic scaffold”
Beginning with “Human DNA sequence from”
Including “genomic DNA”
Including “BAC clone”
Including “PAC clone”
Including “cosmid”
Including “complete genome”
Ending with “complete sequence”
Including “genomic sequence”
Including “exon”
A “HIT LENGHT (sequence length of the hit sequence) of not less than 50000 obtained by this analysis

The annotation information obtained by this analysis is shown in Tables 6-1 to 6-28.

TABLE 6-1

| SEQ ID | | TOP HIT | 2nd HIT | 3rd HIT |
|---|---|---|---|---|
| NO: | FLJ No. | nr Definition | nr Definition | nr Definition |
| 1 | FLJ21182 | ref\|NP_004359.1\| calponin 2 isoform a | emb\|CAG46609.1\| CNN2 [Homo | dbj\|BAD96644.1\| calponin 2 |
| | | [Homo sapiens]¥ emb\|CAH89421.1\| | sapiens]¥ gb\|AAX36458.1\| calponin | isoform a variant [Homo |
| | | hypothetical protein [Pongo pygmaeus]¥ | 2 [synthetic construct] | sapiens] |
| | | sp\|Q99439\|CNN2_HUMAN | | |
| | | Calponin-2 (Calponin H2, smooth | | |
| | | muscle) (Neutral | | |
| | | calponin)¥ dbj\|BAA12090.1\| neutral | | |
| | | calponin [Homo sapiens] | | |
| 2 | FLJ38597 | ref\|XP_865992.1\| PREDICTED: similar | dbj\|BAB26278.1\| unnamed protein | gb\|AAL36150.1\| smoothelin- |
| | | to smoothelin isoform b isoform 5 | product [Mus musculus] | B3 [Homo sapiens] |
| | | [Canis femiliaris] | | |
| 3 | FLJ13700 | gb\|AAY24229.1\| unknown [Homo | ref\|XP_515478.1\| PREDICTED: | ref\|NP_003119.1\| spectrin, |
| | | sapiens] | hypothetical protein XP_515478 | beta, non-erythrocytic 1 |
| | | | [Pan troglodytes] | isoform 1 [Homo |
| | | | | sapiens]¥ sp\|Q01082\|SPTB2_{—} |
| | | | | HUMAN Spectrin beta |
| | | | | chain, brain 1 (Spectrin, non- |
| | | | | erythroid beta chain 1) |
| | | | | (Beta-II spectrin) (Fodrin |
| | | | | beta chain)¥ gb\|AAA60580.1\| |
| | | | | beta-spectrin |
| 4 | FLJ50683 | ref\|NP_005023.2\| plastin 3 [Homo | emb\|CAI39884.1\| plastin 3 (T | gb\|AAX36165.1\| plastin 3 |
| | | sapiens]¥ gb\|AAH39049.1\| Plastin 3 | isoform) [Homo | [synthetic construct] |
| | | [Homo sapiens]¥ gb\|AAH56898.1\| | sapiens]¥ sp\|P13797\|PLST_HUMAN | |
| | | Plastin [Homo | T-plastin (Plastin-3) | |
| | | sapiens]¥ gb\|AAX42595.1\| plastin 3 | | |
| | | [synthetic construct] | | |

| SEQ ID | 4th HIT | 5th HIT |
|---|---|---|
| NO: | nr Definition | nr Definition |
| 1 | emb\|CAG46630.1\| | emb\|CAA79599.1\| h2-calponin |
| | CNN2 [Homo sapiens] | [Sus scrofa]¥ |
| | | sp\|Q08094\|CNN2_PIG |
| | | Calponin-2 (Calponin H2, |
| | | smooth muscle) (Neutral |
| | | calponin) |
| 2 | ref\|NP_599032.1\| | ref\|XP_606421.2\| PREDICTED: |
| | smoothelin isoform a | similar to smoothelin isoform a |
| | [Homo sapiens] | [Bos taurus] |
| 3 | dbj\|BAD92985.1\| | prf\|\|1908227A beta spectrin |
| | spectrin, beta, non- | |
| | erythrocytic 1 isoform 1 | |
| | variant [Homo sapiens] | |
| 4 | ref\|XP_863975.1\| | dbj\|BAD96521.1\| plastin 3 |
| | PREDICTED: similar to | variant [Homo sapiens] |
| | plastin 3 isoform 7 | |
| | [Canis familiaris]¥ | |
| | ref\|XP_538147.2\| | |
| | PREDICTED: similar | |
| | to plastin 3 isoform 1 | |
| | [Canis familiaris] | |

TABLE 6-2

| | | | | |
|---|---|---|---|---|
| 5 | FLJ50199 | gb\|AAH39856.1\| Rac/Cdc42 guanine | dbj\|BAA04985.1\| KIAA0006 [Homo | emb\|CAD97632.1\| |
| | | nucleotide exchange factor 6 [Homo | sapiens] | hypothetical protein |
| | | sapiens]¥ ref\|NP_004831.1\| Rac/Cdc42 | | [Homo sapiens] |
| | | guanine nucleotide exchange factor 6 | | |
| | | [Homo sapiens]¥ emb\|CAI39443.1\| | | |
| | | Rac/Cdc42 guanine nucleotide exchange | | |
| | | factor (GEF) 6 [Homo | | |
| | | sapiens]¥ emb\|CAI42899.1\| Rac/Cdc42 | | |
| | | guanine nucleotide exchange factor (GEF) | | |
| | | 6 [Homo sapiens]¥ | | |
| | | sp\|Q15052\|ARHG6_HUMAN | | |
| | | Rho guanine nucleotide exchange factor 6 | | |
| | | (Rac/Cdc42 guanine nucleotide exchange | | |
| | | factor 6) (PAK-interacting exchange | | |
| | | factor alpha) (Alpha-Pix) (COOL-2) | | |
| 6 | FLJ26440 | ref\|NP_981932.1\| chromosome 6 open | gb\|AAH56253.1\| Chromosome 6 open | emb\|CAI20537.1\| |
| | | reading frame 71 [Homo | reading frame 71 [Homo sapiens] | chromosome 6 open |
| | | sapiens]¥ gb\|AAP22072.1\| iodotyrosine | | reading frame 71 [Homo |
| | | dehalogenase protein [Homo sapiens] | | sapiens] |
| 7 | FLJ21647 | emb\|CAB43293.1\| hypothetical protein | ref\|NP_015561.1\| RAN binding protein | dbj\|BAD96710.1\| RAN |
| | | [Homo sapiens] | 3 isoform RANBP3-d [Homo | binding protein 3 isoform |
| | | | sapiens]¥ dbj\|BAB15106.1\| unnamed | RANBP3-a variant |
| | | | protein product [Homo | [Homo sapiens] |
| | | | sapiens]¥ sp\|Q9H6Z4\|RANB3_HUMAN | |
| | | | Ran-binding protein 3 (RanBP3) | |
| 8 | FLJ26620 | ref\|NP_001738.2\| gelsolin-like capping | ref\|XP_515584.1\| PREDICTED: | gb\|AAX43878.1\| capping |
| | | protein [Homo sapiens]¥ gb\|AAY24128.1\| | hypothetical protein XP_515584 [Pan | protein gelsolin-like |
| | | unknown [Homo sapiens] | troglodytes]¥ gb\|AAH00728.1\| | [synthetic construct] |
| | | | Gelsolin-like capping protein [Homo | |
| | | | sapiens]¥ gb\|AAH14549.1\| Gelsolin- | |
| | | | like capping protein [Homo | |
| | | | sapiens]¥ gb\|AAX32272.1\| capping | |
| | | | protein gelsolin-like [synthetic | |
| | | | construct]¥ sp\|P40121\|CAPG_HUMAN | |
| | | | Macrophage capping protein (Actin- | |
| | | | regulatory protein CAP- | |
| | | | G)¥ gb\|AAA59570.1\| macrophage | |
| | | | capping protein | |

| | | |
|---|---|---|
| 5 | ref\|XP_613352.2\| | ref\|XP_852793.1\| |
| | PREDICTED: similar to | PREDICTED: similar to Rho |
| | Rho guanine nucleotide | guanine nucleotide exchange |
| | exchange factor 6 (PAK- | factor 6 (PAK-interacting |
| | interacting exchange | exchange factor alpha) |
| | factor alpha) (Alpha-Pix) | (Alpha-Pix) (COOL-2) |
| | (COOL-2) isoform 1 | isoform 1 [Canis familiaris] |
| | [Bos taurus] | |
| 6 | ref\|XP_527537.1\| | emb\|CAH89696.1\| |
| | PREDICTED: similar to | hypothetical protein |
| | iodotyrosine dehalogenase | [Pongo pygmaeus] |
| | 1 protein [Pan | |
| | troglodytes] | |
| 7 | ref\|NP_003615.1\| RAN | ref\|XP_533938.2\| |
| | binding protein 3 isoform | PREDICTED: similar to RAN |
| | RANBP3-a [Homo | binding protein 3 isoform |
| | sapiens]¥ emb\|CAA69957.1\| | RANBP3-a isoform 1 |
| | ranbp3 [Homo sapiens] | [Canis familiaris] |
| 8 | ref\|XP_540197.2\| | ref\|NP_001013104.1\| capping |
| | PREDICTED: similar to | protein (actin filament), |
| | Macrophage capping | gelsolin-like (predicted) |
| | protein (Actin-regulatory | [Rattus norvegicus]¥ |
| | protein CAP-G) | gb\|AAH79104.1\| |
| | [Canis familiaris] | Capping protein (actin |
| | | filament), gelsolin-like |
| | | (predicted) [Rattus norvegicus] |

TABLE 6-3

| | | | | |
|---|---|---|---|---|
| 9 | FLJ43792 | ref\|NP_000400.2\| guanylate cyclase activator | gb\|AAA60542.1\| guanylate cyclase | ref\|XP_851487.1\| |
| | | 1A (retina) [Homo sapiens]¥ | activating protein¥ | PREDICTED: similar to |
| | | gb\|AAH31663.1\| Guanylate | gb\|AAA60541.1\| | guanylate cyclase |
| | | cyclase activator 1A (retina) [Homo | guanylate cyclase activating | activator 1A (retina) |
| | | sapiens]¥ emb\|CAB89167.1\| GUCA1A | protein | [Canis familiaris] |
| | | [Homo sapiens]¥ | | |
| | | sp\|P43080\|GUC1A_HUMAN | | |
| | | Guanylyl cyclase-activating protein 1 | | |
| | | (GCAP 1) (Guanylate cyclase activator 1A) | | |
| 10 | FLJ38127 | gb\|AAH11414.1\| C5 orf3 protein [Homo | ref\|NP_061161.1\| hypothetical | ref\|XP_518045.1\| |
| | | sapiens]¥ dbj\|BAB14952.1\| unnamed protein | protein LOC10827 [Homo | PREDICTED: similar to |
| | | product [Homo sapiens] | sapiens]¥ gb\|AAF76523.1\| | chromosome 5 open |
| | | | unknown [Homo sapiens] | reading frame 3 [Pan |
| | | | | troglodytes] |
| 11 | FLJ35050 | ref\|NP_872270.1\| pyruvate kinase 3 isoform | pir\|\|S64635 pyruvate kinase (EC | emb\|CAI29633.1\| |
| | | 2 [Homo sapiens]¥ ref\|NP_872271.1\| | 27.1.40), muscle splice form M1 - | hypothetical protein |
| | | pyruvate kinase 3 isoform 2 [Homo sapiens] | human | [Pongo pygmaeus] |

| | | |
|---|---|---|
| 9 | emb\|CAA64642.1\|guanylyl | gb\|AAB31698.2\| |
| | cyclase-activating protein | photoreceptor guanylyl |
| | [Bos taurus]¥ | cyclase-activating protein; |
| | ref\|NP_776971.1\| | GCAP [Bos taurus] |
| | guanylate cyclase | |
| | activator 1A (retina) [Bos | |
| | taurus]¥ sp\|P46065\|GUC1A_{—} | |
| | BOVIN Guanylyl | |
| | cyclase-activating protein | |
| | 1 (GCAP 1) (Guanylate | |
| | cyclase activator 1A) | |
| 10 | ref\|XP_546285.2\| | ref\|XP_588483.2\| |
| | PREDICTED: similar to | PREDICTED: similar to |
| | CG9590-PA | CG9590-PA [Bos taurus] |
| | [Canis familiaris] | |
| 11 | emb\|CAH93166.1\| | sp\|P11979\|KPYM_FELCA |
| | hypothetical protein | Pyruvate kinase, isozyme |
| | [Pongo pygmaeus] | M1 (Pyruvate kinase muscle |
| | | isozyme) |

TABLE 6-4

| | | | | |
|---|---|---|---|---|
| 12 | FLJ27298 | pdb\|1X86\|H Chain H, Crystal | gb\|AAV38672.1\| ras homolog | ref\|NP_788818.1\| ras homolog |
| | | Structure Of The DhPH | gene family, member A | gene family, member A [Bos |
| | | DOMAINS OF LEUKEMIA- | [synthetic construct]¥ | taurus]¥ ref\|NP_001655.1\| ras |
| | | Assiociated Rhogef In Complex | gb\|AAX43723.1\| ras-like | homolog homolog gene family, |
| | | With Rhoa¥ pdb\|1X86\|F Chain F, | gene family member A | member A [Homo sapiens]¥ |
| | | Crystal Structure Of The DhPH | [synthetic construct]¥ | gb\|AAV38673.1\| ras |
| | | DOMAINS OF LEUKEMIA- | gb\|AAX43206.1\| ras-like | homolog homolog gene family, |
| | | Assiociated Rhogef In Complex | gene family member A | member A [Homo sapiens]¥ |
| | | With Rhoa¥ pdb\|1X86\|D Chain | [synthetic construct]¥ | gb\|AAI02881.1\| Ras |
| | | D, Crystal Structure Of The | gb\|AAX43205.1\| ras-like | homolog homolog gene family, |
| | | DhPH DOMAINS OF | gene family member A | member A [Bos taurus]¥ |
| | | LEUKEMIA- Assiociated Rhogef | [synthetic construct]¥ | gb\|AAH01360.1\| Ras |
| | | In Complex With | gb\|AAX42923.1\| ras-like | homolog homolog gene family, |
| | | Rhoa¥ pdb\|1X86\|B Chain B, | gene family member A | member A [Homo sapiens]¥ |
| | | Crystal Structure Of The DhPH | [synthetic construct]¥ | gb\|AAH05976.1\| Ras |
| | | DOMAINS OF LEUKEMIA- | gb\|AAX36858.1\| ras-like | homolog homolog gene family, |
| | | Assiociated Rhogef In Complex | gene family member A | member A [Homo sapiens]¥ |
| | | With Rhoa | [synthetic construct] | gb\|AAM21117.1\| small |
| | | | | GTP binding protein RhoA (Homo |
| | | | | sapiens]¥ emb\|CAE46190.1\| |
| | | | | hypothetical protein [Homo |
| | | | | sapiens]¥ gb\|AAX41576.1\| ras-like |
| | | | | gene family member A [synthetic |
| | | | | construct]¥ gb\|AAX41339.1\| ras- |
| | | | | like gene family member A |
| | | | | [synthetic construct]¥ |
| | | | | sp\|P61586\|RHOA_HUMAN |
| | | | | Transforming protein RhoA |
| | | | | (H12)¥ sp\|P61585\|RHOA_BOVIN |
| | | | | Transforming protein RhoA (Gb) |
| | | | | (p21)¥ gb\|AAC33178.1\|GTP- |
| | | | | binding protein [Homo |
| | | | | sapiens]¥ emb\|CAA28690.1\| |
| | | | | unnamed protein product [Homo |
| | | | | sapiens]¥ gb\|AAA30409.1\| rho |
| | | | | (Gb) protein |

| | | |
|---|---|---|
| 12 | ref\|NP_476473.1\| aplysia ras-associated | dbj\|BAE38228.1\| |
| | homolog A2 [Rattus norvegicus]¥ | unnamed protein |
| | ref\|NP_058082.2\| ras homolog | product [Mus |
| | gene family, member A [Mus | musculus] |
| | musculus]¥ gb\|AAH68115.1\| Ras homolog | |
| | gene family, member A [Mus | |
| | musculus]¥ dbj\|BAE31372.1\| unnamed protein | |
| | product [Mus musculus]¥ dbj\|BAE29592.1\| | |
| | unnamed protein product [Mus | |
| | musculus]¥ dbj\|BAE42800.1\| unnamed protein | |
| | product [Mus musculus]¥ dbj\|BAC36896.1\| | |
| | unnamed protein product [Mus | |
| | musculus]¥ dbj\|BAC38971.1\| unnamed protein | |
| | product [Mus musculus]¥ gb\|AAH96423.1\| | |
| | Ras homolog gene family, member A [Mus | |
| | musculus]¥ gb\|AAH61732.1\| Aplysia ras- | |
| | associated homolog A2 [Rattus | |
| | norvegicus]¥ gb\|AAK11718.1\| RhoA small | |
| | GTPase [Rattus norvegicus]¥ gb\|AAK11717.1\| | |
| | RhoA small GTPase [Rattus | |
| | norvegicus]¥ sp\|P61589\|RHOA_RAT | |
| | Transforming protein | |
| | RhoA¥ sp\|Q9QUI0\|RHOA_MOUSE | |
| | Transforming protein RhoA¥ gb\|AAD52678.1\| | |
| | Rho family GTPase RhoA [Mus | |
| | musculus]¥ gb\|AAD52677.1\| Rho family | |
| | GTPase RhoA [Mus musculus]¥ | |
| | gb\|AAD52676.1\| Rho family | |
| | GTPase RhoA [Mus musculus]¥ | |
| | gb\|AAD52675.1\| Rho family | |
| | GTPase RhoA [Mus musculus] | |

TABLE 6-5

| | | | | |
|---|---|---|---|---|
| 13 | FLJ26262 | pdb\|1RK4\|B Chain B, Crystal Structure | gb\|AAX36893.1\| | ref\|NP_001279.2\| chloride intracellular channel 1 [Homo |
| | | Of A Soluble Dimeric Form Of Oxidised | chloride | sapiens]¥ gb\|AAD18073.1\| CLIC1 [Homo |
| | | Clic1¥ pdb\|1RK4\|A Chain A, Crystal | intracellular | sapiens]¥ emb\|CAI17825.1\| chloride intracellular channel |
| | | Structure Of A Soluble Dimeric Form Of | channel 1 | 1 [Homo sapiens]¥ emb\|CAI18417.1\| chloride |
| | | Oxidised Clic1 | [synthetic | intracellular channel 1 [Homo sapiens]¥ gb\|AAH64527.1\| |
| | | | construct] | CLIC1 protein [Homo sapiens]¥ emb\|CAB46078.1\| |
| | | | | RNCC protein [Homo sapiens]¥ gb\|AAH95469.1\| |
| | | | | Chloride intracellular channel 1 [Homo |
| | | | | sapiens]¥ emb\|CAG46868.1\| CLIC1 [Homo |
| | | | | sapiens]¥ dbj\|BAB63376.1\| nuclear chloride ion channel |
| | | | | protein [Homo sapiens]¥ gb\|AAD20437.1\| chloride |
| | | | | channel ABP [Homo sapiens]¥ |
| | | | | sp\|O00299\|CLIC1_HUMAN Chloride |
| | | | | intracellular channel protein 1 (Nuclear chloride ion |
| | | | | channel 27) (NCC27) (p64 CLCP) (Chloride channel |
| | | | | ABP) (Regulatory nuclear chloride ion channel protein) |
| | | | | (hRNCC) |
| 14 | FLJ90682 | emb\|CAI16804.1\| CLIC5 [Homo | ref\|NP_058625.1\| | ref\|NP_446055.1\| chloride intracellular channel 5 [Rattus |
| | | sapiens]¥ emb\|CAI21030.1\| CLIC5 | chloride | norvegicus]¥ gb\|AAG49367.1\| chloride intracellular channel |
| | | [Homo sapiens]¥ gb\|AAH35968.1\| | intracellular | 5 [Rattus norvegicus]¥ sp\|Q9EPT8\|CLIC5_RAT Chloride |
| | | Chloride intracellular channel 5 [Homo | channel 5 | intracellular channel protein 5 |
| | | sapiens]¥ dbj\|BAC11444.1\| unnamed | [Homo | |
| | | protein product [Homo | sapiens]¥ | |
| | | sapiens]¥ dbj\|BAD96850.1\| chloride | gb\|AAF66928.1\| | |
| | | intracellular channel 5 variant [Homo | CLIC5 | |
| | | sapiens]¥ dbj\|BAD96264.1\| chloride | [Homo | |
| | | intracellular channel 5 variant [Homo | sapiens] | |
| | | sapiens] | | |
| 15 | FLJ22923 | ref\|NP_005479.1\| target of myb1 [Homo | gb\|AAH46151.1\| | emb\|CAI29664.1\| hypothetical protein [Pongo |
| | | sapiens]¥ emb\|CAI17951.1\| | Target | pygmaeus] |
| | | OTTHUMP00000028777 [Homo | of myb1 | |
| | | sapiens]¥ emb\|CAI21633.1\| | [Homo | |
| | | OTTHUMP00000028777 [Homo | sapiens] | |
| | | sapiens]¥ emb\|CAG30481.1\|TOM1L1 [Homo | | |
| | | sapiens]¥ sp\|O60784\|TOM1_HUMAN Target | | |
| | | of Myb protein 1¥ emb\|CAA07362.1\| TOM1 | | |
| | | [Homo sapiens] | | |

| | | |
|---|---|---|
| 13 | gb\|AAD26137.1\| nuclear | dbj\|BAD97099.1\| |
| | chloride channel [Homo | chloride intracellular |
| | sapiens]¥ gb\|AAC25675.1\| | channel 1 variant |
| | nuclear chloride ion | [Homo sapiens] |
| | channel protein [Homo | |
| | sapiens] | |
| 14 | ref\|NP_766209.1\| chloride | sp\|Q9NZA1\|CLIC5_HUMAN |
| | intracellular channel 5 | Chloride |
| | [Mus musculus]¥ | intracellular channel |
| | gb\|AAH64037.1\| | protein 5 |
| | Chloride intracellular | |
| | channel 5 [Mus | |
| | musculus]¥ dbj\|BAE33875.1\| | |
| | unnamed protein | |
| | product [Mus | |
| | musculus]¥ dbj\|BAC32769.1\| | |
| | unnamed protein | |
| | product [Mus musculus]¥ | |
| | sp\|Q8BXK9\|CLIC5_MOUSE | |
| | Chloride | |
| | intracellular channel | |
| | protein 5 | |
| 15 | emb\|CAH91718.1\| | ref\|NP_001030187.1\| |
| | hypothetical protein | target of myb1 [Bos |
| | [Pongo pygmaeus] | taurus]¥ gb\|AAX31362.1\| |
| | | target of myb1 |
| | | [Bos taurus] |

TABLE 6-6

| | | | | |
|---|---|---|---|---|
| 16 | FLJ22871 | dbj\|BAB33335.1\| KIAA1665 | ref\|NP_612211.1\| polymerase (RNA) | ref\|NP_084505.2\| polymerase (RNA) |
| | | protein [Homo sapiens] | III (DNA dependent polypeptide H (22.9 | III (DNA dependent) polypeptide H |
| | | | kD) isoform a [Homo sapiens]¥ | [Mus musculus]¥ gb\|AAH10793.1\| |
| | | | ref\|NP_001018060.1\| polymerase | Polymerase (RNA) III (DNA |
| | | | (RNA) III (DNA dependent) polypeptide | dependent) polypeptide H [Mus |
| | | | H (22.9 kD) isoform a [Homo | musculus]¥ dbj\|BAB31893.2\| |
| | | | sapiens]¥ emb\|CAB46023.1\| | unnamed protein product [Mus |
| | | | OTTHUMP00000028768 [Homo sapiens]¥ | musculus]¥ ref\|XP_216998.1\| |
| | | | emb\|CAG30345.1\| dJ347H13.5 [Homo | PREDICTED: similar to Polymerase |
| | | | sapiens]¥ gb\|AAM18217.1\| RNA | (RNA) III (DNA dependent) polypeptide |
| | | | polymerase III subunit RPC8 [Homo | H [Rattus norvegicus]¥ |
| | | | sapiens]¥ gb\|AAH88367.1\| | sp\|Q9D2C6\|RPC8_MOUSE |
| | | | Polymerase (RNA) III (DNA dependent) | DNA-dependent RNA polymerase |
| | | | polypeptide H (22.9 kD), isoform a | III subunit 22.9 kDa polypeptide (RPC8) |
| | | | [Homo sapiens]¥ | |
| | | | sp\|Q9Y535\|RPC8_HUMAN | |
| | | | DNA-dependent RNA polymerase III | |
| | | | subunit 22.9 kDa polypeptide (RPC8) | |
| 17 | FLJ20398 | gb\|AAH53589.1\| Ubiquitin- | emb\|CAH93235.1\| hypothetical protein | emb\|CAF25307.1\| ubiquitin-like protein |
| | | like 4 [Homo sapiens]¥ | [Pongo pygmaeus] | GDX [Mus musculus] |
| | | gb\|AAH43346.1\| Ubiquitin- | | |
| | | like 4 [Homo sapiens]¥ | | |
| | | ref\|NP_055050.1\| ubiquitin- | | |
| | | like 4 [Homo sapiens]¥ | | |
| | | emb\|CAI43235.1\| ubiquitin- | | |
| | | like 4 [Homo sapiens]¥ | | |
| | | gb\|AAA92650.1\| ubiquitin- | | |
| | | like protein [Homo sapiens]¥ | | |
| | | sp\|P11441\|UBL4_HUMAN | | |
| | | Ubiquitin-like protein 4 | | |
| | | (Ubiquitin-like protein GDX)¥ | | |
| | | gb\|AAA36790.1\| ubiquitin- | | |
| | | like protein | | |
| 18 | FLJ35377 | gb\|AAC05812.1\| Gene product | ref\|NP_061989.2\| ubiquitin-binding | ref\|NP_613055.2\| ubiquitin-binding |
| | | with similarity to Ubiquitin | protein homolog [Homo sapiens] | protein homolog [Mus musculus] |
| | | binding enzyme [Homo sapiens] | | |
| 19 | FLJ42145 | gb\|AAC05812.1\| Gene product | ref\|NP_061989.2\| ubiquitin-binding | ref\|XP_536933.1\| PREDICTED: similar |
| | | with similarity to Ubiquitin | protein homolog [Homo sapiens] | to ubiquitin-binding protein homolog |
| | | binding enzyme [Homo sapiens] | | isoform 1 [Canis familiaris] |
| 20 | FLJ26144 | dbj\|BAD93141.1\| glucosamine- | ref\|NP_612208.1\| glucosamine-6- | dbj\|BAB70977.1\| unnamed protein |
| | | 6-phosphate deaminase 2 variant | phosphate deaminase 2 [Homo sapiens]¥ | product [Homo sapiens] |
| | | [Homo sapiens] | gb\|AAL95691.1\| glucosamine-6-phosphate | |
| | | | isomerase SB52 [Homo sapiens] | |

| | | |
|---|---|---|
| 16 | dbj\|BAE31279.1\| unnamed | ref\|XP_849136.1\| |
| | protein product [Mus musculus] | PREDICTED: similar to |
| | | polymerase (RNA) III |
| | | (DNA dependent) |
| | | polypeptide H isoform |
| | | 1 [Canis familiaris] |
| 17 | ref\|NP_663380.1\| ubiquitin-like 4 | ref\|XP_215228.1\| |
| | [Mus musculus]¥ | PREDICTED: similar to |
| | gb\|AAH10817.1\| | Ubiquitin-like protein 4 |
| | Ubiquitin-like 4 [Mus | (Ubiquitin-like protein |
| | musculus]¥ dbj\|BAE26908.1\| | GDX) [Rattus |
| | unnamed protein product [Mus | norvegicus] |
| | musculus]¥ sp\|P21126\|UBL4_{—} | |
| | MOUSE Ubiquitin-like protein 4 | |
| | (Ubiquitin-like protein | |
| | GDX)¥ gb\|AAA40520.1\| | |
| | housekeeping protein DXS254E | |
| | (GdX) | |
| 18 | ref\|XP_536933.1\| PREDICTED: | gb\|AAH11313.1\| |
| | similar to ubiquitin-binding | D7Wsu128e protein |
| | protein homolog isoform 1 | [Mus musculus] |
| | [Canis familiaris] | |
| 19 | ref\|NP_613055.2\| ubiquitin- | gb\|AAH11313.1\| |
| | binding protein homolog [Mus | D7Wsu128e protein |
| | musculus] | [Mus musculus] |
| 20 | gb\|AAH15532.1\| Glucosamine- | ref\|XP_849417.1\| |
| | 6-phosphate deaminase 2 | PREDICTED: similar to |
| | [Homo sapiens] | glucosamine-6- |
| | | phosphate deaminase |
| | | 2 isoform 1 [Canis |
| | | familiaris] |

TABLE 6-7

| | | | | |
|---|---|---|---|---|
| 21 | FLJ26374 | ref\|NP_000166.2\| glucose phosphate | gb\|AAP36518.1\| Homo | pdb\|1JLH\|D Chain D, Human |
| | | isomerase [Homo sapiens]¥ gb\|AAH04982.1\| | sapiens glucose phosphate | Glucose-6-Phosphate |
| | | Glucose phosphate isomerase [Homo | isomerase [synthetic | Isomerase¥ pdb\|1JLH\|C |
| | | sapiens]¥ gb\|AAP72966.1\| glucose | construct]¥ gb\|AAX28982.1\| | Chain C, Human Glucose- |
| | | phosphate isomerase [Homo | glucose phosphate | 6-Phosphate |
| | | sapiens]¥ sp\|P06744\|G6PI_HUMAN | isomerase [synthetic | Isomerase¥ pdb\|1JLH\|B |
| | | Glucose-6-phosphate isomerase (GPI) | construct]¥ gb\|AAX28981.1\| | Chain B, Human Glucose-6- |
| | | (Phosphoglucose isomerase) (PGI) | glucose phosphate | Phosphate Isomerase¥ |
| | | (Phosphohexose isomerase) (PHI) | isomerase [synthetic | pdb\|1JLH\|A Chain A, |
| | | (Neuroleukin) (NLK) (Sperm antigen 36) | construct] | Human Glucose-6- |
| | | (SA-3))¥ pdb\|1NUH\|A Chain A, The Crystal | | Phosphate Isomerase |
| | | Structure Of Human Phosphoglucose | | |
| | | Isomerase Complexed With 5- | | |
| | | Phosphoarabinonate¥ pdb\|1IRI\|D Chain D, | | |
| | | Crystal Structure Of Human Autocrine | | |
| | | Motility Factor Complexed With An | | |
| | | Inhibitor¥ pdb\|1IRI\|C Chain C, Crystal | | |
| | | Structure Of Human Autocrine Motility | | |
| | | Factor Complexed With An | | |
| | | Inhibitor¥ pdb\|1IRI\|B Chain B, Crystal | | |
| | | Structure Of Human Autocrine Motility | | |
| | | Factor Complexed With An | | |
| | | Inhibitor¥ pdb\|1IRI\|A Chain A, Crystal | | |
| | | Structure Of Human Autocrine Motility | | |
| | | Factor Complexed With An | | |
| | | Inhibitor¥ pdb\|1JIQ\|D Chain D, Crystal | | |
| | | Structure Of Human Autocrine Motility | | |
| | | Factor¥ pdb\|1JIQ\|C Chain C, Crystal | | |
| | | Structure Of Human Autocrine Motility | | |
| | | Factor¥ pdb\|1JIQ\|B Chain B, Crystal | | |
| | | Structure Of Human Autocrine Motility | | |
| | | Factor¥ pdb\|1JIQ\|A Chain A, Crystal | | |
| | | Structure Of Human Autocrine Motility | | |
| | | Factor | | |

| | | |
|---|---|---|
| 21 | pdb\|1IAT\|A Chain A, Crystal | gb\|AAF22645.1\| sperm |
| | Structure Of Human | antigen-36 [Homo sapiens] |
| | Phosphoglucose Isomerase | |
| | NEUROLEUKINAUTOCRINE | |
| | MOTILITY | |
| | FACTORMATURATION | |
| | Factor | |

TABLE 6-8

| | | | | |
|---|---|---|---|---|
| 22 | FLJ26371 | gb\|AAV38570.1\| lactate dehydrogenase B | gb\|AAX29227.1\| lactate | pdb\|1I0Z\|B Chain B, Human |
| | | [Homo sapiens]¥ gb\|AAV38569.1\| lactate | dehydrogenase B [synthetic | Heart L-Lactate |
| | | dehydrogenase B [Homo | construct] | Dehydrogenase H Chain, |
| | | sapiens]¥ ref\|NP_002291.1\| lactate | | Ternary Complex With Nadh |
| | | dehydrogenase B [Homo | | And Oxamate¥ pdb\|1I0Z\|A |
| | | sapiens]¥ dbj\|BAE01709.1\| unnamed protein | | Chain A, Human Heart L- |
| | | product [Macaca | | Lactate Dehydrogenase H |
| | | fascicularis]¥ gb\|AAO85222.1\| | | Chain, Ternary Complex |
| | | transformation-associated protein 5 [Homo | | With Nadh And Oxamate |
| | | sapiens]¥ gb\|AAX41164.1\| lactate | | |
| | | dehydrogenase B [synthetic | | |
| | | construct]¥ gb\|AAX41163.1\| lactate | | |
| | | dehydrogenase B [synthetic | | |
| | | construct]¥ gb\|AAH71860.1\| Lactate | | |
| | | dehydrogenase B [Homo | | |
| | | sapiens]¥ gb\|AAH02362.1\| Lactate | | |
| | | dehydrogenase B [Homo | | |
| | | sapiens]¥ gb\|AAH15122.1\| Lactate | | |
| | | dehydrogenase B [Homo | | |
| | | sapiens]¥ sp\|P07195\|LDHB_HUMAN L- | | |
| | | lactate dehydrogenase B chain (LDH-B) | | |
| | | (LDH heart subunit) (LDH- | | |
| | | H)¥ emb\|CAA68701.1\| unnamed protein | | |
| | | product [Homo sapiens]¥ emb\|CAA32033.1\| | | |
| | | lactate dehydrogenase B [Homo sapiens] | | |

| | | |
|---|---|---|
| 22 | ref\|XP_534868.1\| | gb\|AAX32621.1\| lactate |
| | PREDICTED: similar to L- | dehydrogenase B [synthetic |
| | lactate dehydrogenase B | construct] |
| | chain (LDH-B) (LDH heart | |
| | subunit) (LDH-H) [Canis | |
| | familiaris] | |

TABLE 6-9

| | | | | |
|---|---|---|---|---|
| 23 | FLJ45688 | ref\|NP_002698.1\| protein phosphatase 1G | dbj\|BAE01873.1\| unnamed | ref\|XP_532910.2\| |
| | | [Homo sapiens]¥ ref\|NP_817092.1\| protein | protein product [Macaca | PREDICTED: similar to |
| | | phosphatase 1G [Homo | fascicularis] | protein phosphatase 1G |
| | | sapiens]¥ gb\|AAH00057.1\| Protein | | isoform 2 [Canis familiaris] |
| | | phosphatase 1G [Homo | | |
| | | sapiens]¥ gb\|AAH22061.1\| Protein | | |
| | | phosphatase 1G [Homo | | |
| | | sapiens]¥ emb\|CAA74245.1\| protein | | |
| | | phosphatase 2C gamma [Homo | | |
| | | sapiens]¥ gb\|AAP36122.1\| protein | | |
| | | phosphatase 1G (formerly 2C), magnesium- | | |
| | | dependent; gamma isoform [Homo | | |
| | | sapiens]¥ emb\|CAG33340.1\| PPM1G [Homo | | |
| | | sapiens]¥ gb\|AAY14846.1\| unknown [Homo | | |
| | | sapiens]¥ gb\|AAX42118.1\| protein | | |
| | | phosphatase 1G magnesium-dependent | | |
| | | gamma isoform [synthetic | | |
| | | construct]¥ gb\|AAX42117.1\| protein | | |
| | | phosphatase 1G magnesium-dependent | | |
| | | gamma isoform [synthetic | | |
| | | construct]¥ sp\|O15355\|PP2CG_HUMAN | | |
| | | Protein phosphatase 2C gamma isoform | | |
| | | (PP2C-gamma) (Protein phosphatase | | |
| | | magnesium-dependent 1 gamma) (Protein | | |
| | | phosphatase 1C) | | |
| 24 | FLJ38620 | gb\|AAG17244.1\| unknown [Homo sapiens] | dbj\|BAC04654.1\| unnamed | gb\|AAH67256.1\|RPRC1 |
| | | | protein product [Homo | protein [Homo sapiens] |
| | | | sapiens] | |

| | | |
|---|---|---|
| 23 | gb\|AAI03459.1\| Unknown | gb\|AAH62083.1\| Protein |
| | (protein for MGC: 128712) | phosphatase 1G (formerly |
| | [Bos taurus] | 2C), magnesium-dependent, |
| | | gamma isoform [Rattus |
| | | norvegicus]¥ gb\|AAM90993.1\| |
| | | protein phosphatase |
| | | PP2C gamma [Rattus |
| | | norvegicus]¥ ref\|NP_671742.1\| |
| | | protein phosphatase 1G |
| | | (formerly 2C), magnesium- |
| | | dependent, gamma isoform |
| | | [Rattus norvegicus] |
| 24 | emb\|CAG33535.1\| | gb\|AAH27334.1\| RPRC1 |
| | FLJ10350 [Homo | protein [Homo sapiens] |
| | sapiens]¥ dbj\|BAA91557.1\| | |
| | unnamed protein product | |
| | [Homo sapiens] | |

TABLE 6-10

| | | | | | | |
|---|---|---|---|---|---|---|
| 25 | FLJ26267 | ref\|XP_518797.1\| | dbj\|BAE01655.1\| | emb\|CAH91321.1\| | ref\|XP_861806.1\| | ref\|XP_861777.1\| |
| | | PREDICTED: similar to | unnamed protein | hypothetical protein | PREDICTED: similar to | PREDICTED: similar to |
| | | protein-L-isoaspartate | product [Macaca | [Pongo pygmaeus] | Protein-L-isoaspartate(D- | Protein-L-isoaspartate(D- |
| | | (D-aspartate) O- | fescicularis] | | aspartate) O- | aspartate) O- |
| | | methyltransferase 1 | | | methyltransferase (Protein- | methyltransferase (Protein- |
| | | [Pan troglodytes] | | | beta-aspartate | beta-aspartate |
| | | | | | methyltransferase) (PIMT) | methyltransferase) (PIMT) |
| | | | | | (Protein L-isoaspartyl/D- | (Protein L-isoaspartyl/D- |
| | | | | | aspartyl methyltransferase) | aspartyl methyltransferase) |
| | | | | | (L-isoaspartyl protein | (L-isoaspartyl protein |
| | | | | | carboxyl methyltransferase) | carboxyl methyltransferase) |
| | | | | | isoform 8 [Canis | isoform 7 [Canis familiaris] |
| | | | | | familiaris]¥ ref\|XP_850565.1\| | |
| | | | | | PREDICTED: similar to | |
| | | | | | Protein-L-isoaspartate(D- | |
| | | | | | aspartate) O- | |
| | | | | | methyltransferase (Protein- | |
| | | | | | beta-aspartate | |
| | | | | | methyltransferase) (PIMT) | |
| | | | | | (Protein L-isoaspartyl/D- | |
| | | | | | aspartyl methyltransferase) | |
| | | | | | (L-isoaspartyl protein | |
| | | | | | carboxyl methyltransferase) | |
| | | | | | isoform 6 [Canis familiaris] | |

TABLE 6-11

| | | | | |
|---|---|---|---|---|
| 26 | FLJ26062 | dbj\|BAD93038.1\| | gb\|AAV38791.1\| glyoxalase | gb\|AAV38789.1\| |
| | | glyoxalase I variant | I [Homo sapiens]¥ | glyoxalase I [synthetic |
| | | [Homo sapiens] | gb\|AAV38790.1\| | construct]¥ gb\|AAX43062.1\| |
| | | | glyoxalase I [Homo | glyoxalase I [synthetic |
| | | | sapiens]¥ gb\|AAH01741.1\| | construct]¥ gb\|AAX43061.1\| |
| | | | Glyoxalase I [Homo | glyoxalase I [synthetic |
| | | | sapiens]¥ emb\|CAI21586.1\| | construct] |
| | | | glyoxalase I [Homo | |
| | | | sapiens]¥ gb\|AAB49495.1\| | |
| | | | glyoxalase I [Homo | |
| | | | sapiens]¥ gb\|AAX41429.1\| | |
| | | | glyoxalase I [synthetic | |
| | | | construct]¥ gb\|AAX41428.1\| | |
| | | | glyoxalase I [synthetic | |
| | | | construct] | |
| 27 | FLJ22936 | ref\|NP_665801.1\| | ref\|NP_665799.1\| septin 6 | ref\|NP_055944.2\| |
| | | septin 6 isoform D | isoform A [Homo | septin 6 isoform B |
| | | [Homo sapiens]¥ | sapiens]¥ ref\|NP_665798.1\| | [Homo sapiens]¥ |
| | | emb\|CAI41425.1\| | septin 6 isoform A [Homo | emb\|CAI41426.1\| |
| | | septin 6 [Homo | sapiens]¥ emb\|CAI41428.1\| | septin 6 [Homo |
| | | sapiens]¥ gb\|AAK98551.1\| | septin 6 [Homo | sapiens]¥ gb\|AAH36240.1\| |
| | | SEPTIN6 type V | sapiens]¥ gb\|AAK61492.l\| | Septin 6, isoform |
| | | [Homo sapiens]¥ | septin 6 [Homo | B [Homo sapiens]¥ |
| | | gb\|AAN76547.1\| | sapiens]¥ gb\|AAK98547.1\| | gb\|AAK98548.1\| |
| | | septin 6 [Homo | SEPTIN6 type I [Homo | SEPTIN6 type II |
| | | sapiens]¥ gb\|AAH11922.3\| | sapiens]¥ gb\|AAK98549.1\| | [Homo sapiens]¥ |
| | | Septin 6, isoform | SEPTIN6 type II [Homo | sp\|Q14141\| |
| | | D [Homo sapiens] | sapiens]¥ gb\|AAF97496.1\| | SEPT6_HUMAN |
| | | | septin 6 [Homo sapiens] | Septin-6 |

| | | |
|---|---|---|
| 26 | pdb\|1QIP\|D Chain D Complexed With S-P- | ref\|NP_006699.1\| glyoxalase I |
| | Nitrobenzyloxycarbonylglutathione, Human Glyoxalase I | [Homo sapiens]¥ gb\|AAH15934.1\| |
| | ¥ pdb\|1QIP\|C Chain C Complexed With S-P- | Glyoxalase I [Homo |
| | Nitrobenzyloxycarbonylglutathione, Human Glyoxalase I | sapiens]¥ gb\|AAD38008.1\| |
| | ¥ pdb\|1QIP\|B Chain B Complexed With S-P- | glyoxalase-I [Homo |
| | Nitrobenzyloxycarbonylglutathione, Human Glyoxalase I | sapiens]¥ sp\|Q04760\|LGUL_HUMAN |
| | ¥ pdb\|1QIP\|A Chain A Complexed With S-P- | Lactoylglutathione lyase |
| | Nitrobenzyloxycarbonylglutathione, Human Glyoxalase I | (Methylglyoxalase) |
| | ¥ pdb\|1QIN\|B Chain B Complexed With S-(N-Hydroxy- | (Aldoketomutase) (Glyoxalase I) |
| | N-P- Iodophenylcarbamoyl) Glutathione, Human | (Glx I) (Ketone-aldehyde mutase) |
| | Glyoxalase I ¥ pdb\|1QIN\|A Chain AComplexed With S- | (S-D-lactoylglutathione |
| | (N-Hydroxy-N-P- Iodophenylcarbamoyl) Glutathione, | methylglyoxal |
| | Human Glyoxalase I ¥ pdb\|1FRO\|D Chain D With Benzyl- | lyase)¥ gb\|AAA52565.1\| |
| | Glutathione Inhibitor, Human Glyoxalase I ¥ pdb\|1FRO\|C | glyoxaslase I¥ dbj\|BAA02572.1\| |
| | Chain C With Benzyl-Glutathione Inhibitor, Human | lactoyl glutathione lyase [Homo |
| | Glyoxalase I ¥ pdb\|1FRO\|B Chain B With Benzyl- | sapiens] |
| | Glutathione Inhibitor, Human Glyoxalase I ¥ pdb\|1FRO\|A | |
| | Chain A With Benzyl-Glutathione Inhibitor, Human | |
| | Glyoxalase I | |
| 27 | dbj\|BAA09477.1\| KIAA0128 [Homo sapiens] | emb\|CAI41427.1\| septin 6 [Homo |
| | | sapiens] |

TABLE 6-12

| | | | | |
|---|---|---|---|---|
| 28 | FLJ43223 | ref\|NP_003671.1\| tyrosyl-tRNA | dbj\|BAD97328.1\| tyrosyl-tRNA synthetase variant | emb\|CAH91825.1\| |
| | | synthetase [Homo | [Homo sapiens] | hypothetical protein |
| | | sapiens]¥ gb\|AAH16689.1\| Tyrosyl- | | [Pongo pygmaeus] |
| | | tRNA synthetase [Homo | | |
| | | sapiens]¥ gb\|AAH01933.1\| Tyrosyl- | | |
| | | tRNA synthetase [Homo | | |
| | | sapiens]¥ gb\|AAH04151.1\| Tyrosyl- | | |
| | | tRNA synthetase [Homo | | |
| | | sapiens]¥ sp\|P54577\|SYYC_HUMAN | | |
| | | Tyrosyl-tRNA synthetase, | | |
| | | cytoplasmic (Tyrosyl-tRNA ligase) | | |
| | | (TyrRS)¥ gb\|AAB88409.1\| tyrosyl- | | |
| | | tRNA synthetase [Homo sapiens] | | |
| 29 | FLJ26102 | emb\|CAH91134.1\| hypothetical protein | ref\|NP_001850.1\| solute carrier family 31 (copper | dbj\|BAD96586.1\| |
| | | [Pongo pygmaeus] | transporters), member 1 [Homo | solute carrier family |
| | | | sapiens]¥ gb\|AAH13611.1\| Solute carrier family 31 | 31 (copper |
| | | | (copper transporters), member 1 [Homo | transporters), |
| | | | sapiens]¥ emb\|CAI10965.1\| solute carrier family 31 | member 1 variant |
| | | | (copper transporters), member 1 [Homo | [Homo sapiens] |
| | | | sapiens]¥ emb\|CAD38549.1\| hypothetical protein | |
| | | | [Homo sapiens]¥ sp\|O15431\|COPT1_HUMAN | |
| | | | High-affinity copper uptake protein 1 (hCTR1) | |
| | | | (Copper transporter 1) (Solute carrier family 31 | |
| | | | member 1)¥ gb\|AAB66306.1\| high-affinity copper | |
| | | | uptake protein [Homo sapiens] | |
| 30 | FLJ25218 | ref\|XP_522457.1\| PREDICTED: similar | gb\|AAH70232.1\| Hypothetical protein MGC14817 | gb\|AAI07781.1\| |
| | | to hypothetical protein MGC14817 | [Homo sapiens]¥ dbj\|BAC03699.1\| unnamed | Hypothetical protein |
| | | [Pan troglodytes] | protein product [Homo sapiens]¥ ref\|NP_115714.1\| | LOC84298 [Homo |
| | | | hypothetical protein LOC84298 [Homo | sapiens] |
| | | | sapiens]¥ gb\|AAH06002.1\| Hypothetical protein | |
| | | | MGC14817 [Homo sapiens] | |

| | | |
|---|---|---|
| 28 | ref\|XP_524651.1\| PREDICTED: | dbj\|BAE41320.1\| unnamed |
| | tyrosyl-tRNA synthetase [Pan | protein product [Mus musculus] |
| | troglodytes] | |
| 29 | ref\|XP_538800.1\| PREDICTED: | ref\|XP_520197.1\| PREDICTED: |
| | similar to High-affinity | similar to High-affinity |
| | copper uptake protein 1 | copper uptake protein 1 |
| | (hCTR1) (Copper transporter 1) | (hCTR1) (Copper transporter 1) |
| | (Solute carrier family 31, | [Pan troglodytes] |
| | member 1) [Canis familiaris] | |
| 30 | ref\|XP_880473.1\| PREDICTED: | ref\|XP_880329.1\| PREDICTED: |
| | hypothetical protein XP_875380 | similar to C49H3.3 isoform 2 |
| | isoform 3 [Bos taurus]¥ | [Bos taurus] |
| | ref\|XP_587662.1\| | |
| | PREDICTED: hypothetical | |
| | protein XP_587662 isoform 1 | |
| | [Bos taurus] | |

TABLE 6-13

| | | | | | | |
|---|---|---|---|---|---|---|
| 31 | FLJ45675 | ref\|NP_076957.3\|hypothetical | dbj\|BAB85036.1\|unnamed | gb\|AAH00636.2\|C17 orf39 | ref\|XP_586478.2\| | gb\|AAH46821.1\|RIKEN cDNA |
| | | protein LOC79018 [Homo | protein product [Homo | protein [Homo sapiens] | PREDICTED: similar to | 4933439F18 [Mus |
| | | sapiens]¥ gb\|AAH41829.1\| | sapiens] | | Protein C17 orf39 homolog | musculus]¥ gb\|AAH44901.1\| |
| | | Hypothetical protein LOC79018 | | | [Bos taurus] | RIKEN cDNA 4933439F18 [Mus |
| | | [Homo | | | | musculus]¥ emb\|CAI24078.1\| |
| | | sapiens]¥ sp\|Q8IVV7\|CQ039_HUMAN | | | | novel protein [Mus |
| | | Protein C17 orf39 | | | | musculus]¥ dbj\|BAE25186.1\| |
| | | | | | | unnamed protein product [Mus |
| | | | | | | musculus]¥ dbj\|BAE37858.1\| |
| | | | | | | unnamed protein product [Mus |
| | | | | | | musculus]¥ dbj\|BAB30608.1\| |
| | | | | | | unnamed protein product [Mus |
| | | | | | | musculus]¥ dbj\|BAC36995.1\| |
| | | | | | | unnamed protein product [Mus |
| | | | | | | musculus]¥ dbj\|BAC30346.1\| |
| | | | | | | unnamed protein product [Mus |
| | | | | | | musculus]¥ dbj\|BAB27989.1\| |
| | | | | | | unnamed protein product [Mus |
| | | | | | | musculus]¥ sp\|Q9CPY6\|CQ039_MOUSE |
| | | | | | | Protein C17 orf39 homolog |
| 32 | FLJ25918 | gb\|AAH07364.1\|HSCARG protein | ref\|XP_547146.1\|PREDICTED: | ref\|XP_886066.1\|PREDICTED: | ref\|NP_080669.1\| | ref\|XP_213217.3\|PREDICTED: |
| | | [Homo sapiens]¥ gb\|AAH02927.1\| | hypothetical protein | hypothetical protein XP_880973 | hypothetical protein | similar to RIKEN cDNA |
| | | HSCARG protein [Homo | XP_547146 [Canis familiaris] | isoform 5 [Bos | LOC67824 [Mus | 1110025F24 [Rattus norvegicus] |
| | | sapiens]¥ gb\|AAG09721.1\| | | taurus]¥ ref\|XP_614462.2\| | musculus]¥ gb\|AAH30039.1\| | |
| | | HSCARG [Homo | | PREDICTED: hypothetical | RIKEN cDNA 1110025F24 | |
| | | sapiens]¥ ref\|NP_065728.1\| | | protein XP_614462 isoform 1 | [Mus musculus] | |
| | | hypothetical protein LOC57407 | | [Bos taurus]¥ ref\|XP_872927.1\| | | |
| | | [Homo sapiens] | | PREDICTED: hypothetical | | |
| | | | | protein XP_867834 isoform 2 [Bos | | |
| | | | | taurus] | | |
| 33 | FLJ46709 | ref\|NP_950251.1\|hypothetical | dbj\|BAD74069.1\|C21 orf25 | dbj\|BAA95528.1\|C21 orf258 | ref\|XP_607988.2\| | ref\|XP_544899.2\|PREDICTED: |
| | | protein LOC25966 [Homo | [Homo | [Homo sapiens] | PREDICTED: hypothetical | hypothetical protein XP_544899 |
| | | sapiens]¥ ref\|XP_032945.4\| | sapiens]¥ dbj\|BAD74068.1\| | | protein XP_607988 [Bos | [Canis familiaris] |
| | | chromosome 21 open reading | C21 orf25 [Homo sapiens] | | taurus] | |
| | | frame 25 [Homo sapiens] | | | | |

TABLE 6-14

| | | | | |
|---|---|---|---|---|
| 35 | FLJ40377 | gb\|AAH29811.1\|FLJ32658 | dbj\|BAB71384.1\|unnamed | ref\|XP_512817.1\|PREDICTED: |
| | | protein [Homo sapiens] | protein product [Homo sapiens] | similar to hypothetical protein |
| | | | | FLJ32658 [Pan troglodytes] |
| 36 | FLJ25845 | ref\|NP_775104.1\|armadillo repeat | emb\|CAH72189.1\|novel protein | dbj\|BAC05389.1\|unnamed |
| | | containing 3 [Homo | [Homo sapiens] | protein product [Homo sapiens] |
| | | sapiens]¥ gb\|AAH39312.1\| | | |
| | | Armadillo repeat containing 3 | | |
| | | [Homo sapiens] | | |
| 37 | FLJ23662 | emb\|CAH92064.1\|hypothetical | ref\|NP_060053.2\|DIPB protein | emb\|CAB65108.1\|DIPB protein |
| | | protein [Pongo pygmaeus] | [Homo | [Homo sapiens] |
| | | | sapiens]¥ gb\|AAH24031.1\|DIPB | |
| | | | protein [Homo | |
| | | | sapiens]¥ gb\|AAH13166.1\|DIPB | |
| | | | protein [Homo | |
| | | | sapiens]¥ sp\|Q96DX7\|TRI44_HUMAN | |
| | | | Tripartite motif protein 44 | |
| | | | (DIPB protein) | |
| 38 | FLJ12668 | dbj\|BAD97212.1\|activating | ref\|NP_079273.2\|activating | gb\|AAH33891.1\|Activating |
| | | transcription factor 7 interacting | transcription factor 7 interacting | transcription factor 7 interacting |
| | | protein 2 variant [Homo sapiens] | protein 2 [Homo sapiens] | protein 2 [Homo |
| | | | | sapiens]¥ gb\|AAT66299.1\| |
| | | | | MBD1-containing chromatin |
| | | | | associated factor 2 [Homo |
| | | | | sapiens] |
| 39 | FLJ90085 | dbj\|BAC11064.1\|unnamed | ref\|NP_116229.1\|hypothetical | dbj\|BAC11144.1\|unnamed |
| | | protein product [Homo sapiens] | protein LOC84926 [Homo | protein product [Homo sapiens] |
| | | | sapiens]¥ dbj\|BAB55311.1\| | |
| | | | unnamed protein product [Homo | |
| | | | sapiens] | |

| | | |
|---|---|---|
| 35 | ref\|NP_653289.2\|hypothetical | ref\|XP_541495.2\|PREDICTED: |
| | protein LOC147872 [Homo | similar to dynactin 1 [Canis |
| | sapiens]¥ dbj\|BAC87306.1\| | familiaris] |
| | unnamed protein product | |
| | [Homo sapiens] | |
| 36 | ref\|XP_535165.2\|PREDICTED: | ref\|XP_622876.1\|PREDICTED: |
| | similar to armadillo repeat | similar to armadillo repeat |
| | containing 3 [Canis familiaris] | containing 3 [Mus musculus] |
| 37 | gb\|AAH45602.1\|Trim44 | sp\|Q9QXA7\|TRI44_MOUSE |
| | protein [Mus | Tripartite motif protein 44 (DIPB |
| | musculus]¥ gb\|AAH39979.1\| | protein) (Mc7 protein) |
| | Trim44 protein [Mus | |
| | musculus] | |
| 38 | ref\|XP_523295.1\|PREDICTED: | gb\|AAH69730.1\|ATF7IP2 |
| | similar to activating | protein [Homo |
| | transcription factor 7 | sapiens]¥ gb\|AAH69713.1\| |
| | interacting protein 2 [Pan | ATF7IP2 protein [Homo |
| | troglodytes] | sapiens]¥ gb\|AAH69695.1\| |
| | | ATF7IP2 protein [Homo |
| | | sapiens] |
| 39 | ref\|XP_484507.1\|PREDICTED: | gb\|AAH08150.1\|BC008150 |
| | hypothetical protein | protein [Mus musculus] |
| | XP_484507 [Mus musculus] | |

TABLE 6-15

| | | | | |
|---|---|---|---|---|
| 40 | FLJ90364 | ref\|NP_932156.1\|nudix-type motif 9 isoform | gb\|AAP36171.1\|Homo | gb\|AAM46068.1\|NUDT10 |
| | | a [Homo sapiens]¥ ref\|NP_076952.1\|nudix- | sapiens nudix | [Homo sapiens] |
| | | type motif 9 isoform a [Homo | (nucleoside diphosphate | |
| | | sapiens]¥ gb\|AAH00542.1\|Nudix-type motif | linked moiety X)-type | |
| | | 9, isoform a [Homo | motif 9 [synthetic | |
| | | sapiens]¥ gb\|AAQ89480.1\|NUDT9 [Homo | construct]¥ gb\|AAX43771.1\| | |
| | | sapiens]¥ gb\|AAK07671.1\|ADP-ribose | nudix-type motif 9 | |
| | | pyrosphosphatase NUDT9 [Homo | [synthetic construct] | |
| | | sapiens]¥ sp\|Q9BW91\|NUDT9_HUMAN | | |
| | | ADP-ribose pyrophosphatase, mitochondrial | | |
| | | precursor (ADP-ribose diphosphatase) | | |
| | | (Adenosine diphosphoribose | | |
| | | pyrophosphatase) (ADPR-PPase) (ADP- | | |
| | | ribose phosphohydrolase) (Nucleoside | | |
| | | diphosphate-linked moiety X motif 9) (Nudix | | |
| | | motif 9) | | |
| 41 | FLJ90401 | ref\|XP_517396.1\|PREDICTED: similar to | dbj\|BAC11225.1\| | ref\|XP_545023.2\| |
| | | ELOVL family member 6, elongation of long | unnamed protein | PREDICTED: similar to |
| | | chain fatty acids (FEN1/Elo2, SUR4/Elo3- | product [Homo sapiens] | ELOVL family member 6, |
| | | like, yeast); long-chain fatty-acyl elongase | | elongation of long chain |
| | | [Pan troglodytes]¥ ref\|NP_076995.1\|ELOVL | | fatty acids (FEN1/Elo2, |
| | | family member 6, elongation of long chain | | SUR4/Elo3-like, yeast) |
| | | fatty acids (FEN1/Elo2, SUR4/Elo3-like, | | [Canis familiaris] |
| | | yeast) [Homo sapiens]¥ gb\|AAH01305.1\| | | |
| | | ELOVL6 protein [Homo | | |
| | | sapiens]¥ dbj\|BAB15632.1\|unnamed protein | | |
| | | product [Homo sapiens] | | |

| | | |
|---|---|---|
| 40 | dbj\|BAC11601.1\|unnamed protein | dbj\|BAB55021.1\| |
| | product [Homo sapiens] | unnamed protein |
| | | product [Homo sapiens] |
| 41 | ref\|NP_569717.1\|ELOVL family | dbj\|BAB69888.1\|fatty |
| | member 6, elongation of long chain | acid elongase 2 [Rattus |
| | fatty acids [Mus | norvegicus]¥ ref\|NP_599210.1\| |
| | musculus]¥ gb\|AAI00577.1\|Elovl6 | ELOVL family |
| | protein [Mus | member 6, elongation of |
| | musculus]¥ gb\|AAH98492.1\|Elovl6 | long chain fatty acids |
| | protein [Mus | [Rattus norvegicus] |
| | musculus]¥ gb\|AAH51041.1\|Elovl6 | |
| | protein [Mus | |
| | musculus]¥ dbj\|BAE39469.1\|unnamed | |
| | protein product [Mus | |
| | musculus]¥ gb\|AAM13450.1\| | |
| | myelination associated SUR4-like | |
| | protein [Mus | |
| | musculus]¥ gb\|AAL14239.1\|long- | |
| | chain fatty-acyl elongase [Mus | |
| | musculus]¥ dbj\|BAB68544.1\|fatty acyl | |
| | elongase [Mus musculus] | |

TABLE 6-16

| | | | | | | |
|---|---|---|---|---|---|---|
| 42 | FLJ25526 | gb\|AAH40496.1\|P25 Protein [Homo | ref\|XP_517605.1\| | ref\|NP_008961.1\|brain-specific protein p25 | ref\|XP_545196.2\| | ref\|NP_878259.1\|tubulin |
| | | sapiens] | PREDICTED: similar to P25 | alpha [Homo sapiens]¥ gb\|AAV38838.1\|brain- | PREDICTED: similar to | polymerization-promoting |
| | | | protein [Pan troglodytes] | specific protein p25 alpha [Homo | Tubulin polymerization- | protein [Mus |
| | | | | sapiens]¥ gb\|AAQ96657.1\|fibroblast growth | promoting protein (TPPP) (25 kDa | musculus]¥ gb\|AAH54803.1\| |
| | | | | factor-2 repression protein-1; FREP1 [Homo | brain-specific protein) | Tubulin polymerization- |
| | | | | sapiens]¥ gb\|AAX41230.1\|brain-specific protein | (p25-alpha) (p24) (p25) [Canis | promoting protein [Mus |
| | | | | p25 alpha [synthetic | familiaris] | musculus]¥ dbj\|BAE24727.1\| |
| | | | | construct]¥ sp\|O94811\|P25A_HUMAN Tubulin | | unnamed protein product [Mus |
| | | | | polymerization-promoting protein (TPPP) (25 kDa | | musculus]¥ |
| | | | | brain-specific protein) (p25-alpha) (p24) | | sp\|Q7TQD2\|P25A_MOUSE |
| | | | | (p25)¥ dbj\|BAA36164.1\|p25 alpha [Homo | | Tubulin polymerization- |
| | | | | sapiens] | | promoting protein (TPPP) |
| 43 | FLJ46896 | ref\|NP_001017995.1\|hypothetical protein | dbj\|BAE37356.1\|unnamed | ref\|NP_796338.2\|hypothetical protein | dbj\|BAE42425.1\|unnamed | dbj\|BAC40843.1\|unnamed |
| | | LOC285590 [Homo sapiens] | protein product [Mus | LOC268396 [Mus musculus] | protein product [Mus | protein product [Mus musculus] |
| | | | musculus]¥ dbj\|BAE41493.1\| | | musculus] | |
| | | | unnamed protein product | | | |
| | | | [Mus musculus] | | | |
| 44 | FLJ46856 | gb\|AAT80901.1\|striated muscle | ref\|XP_536083.2\| | dbj\|BAE37758.1\|unnamed protein product [Mus | ref\|NP_031489.2\|aortic | ref\|XP_343597.2\|PREDICTED: |
| | | preferentially expressed protein [Homo | PREDICTED: similar to aortic | musculus] | preferentially expressed gene | similar to striated muscle- |
| | | sapiens] | preferentially expressed gene | | 1 [Mus | specific serine/threonine protein |
| | | | 1 [Canis familiaris] | | musculus]¥ gb\|AAG34791.1\| | kinase [Rattus norvegicus] |
| | | | | | striated muscle-specific | |
| | | | | | serine/threonine protein | |
| | | | | | kinase [Mus musculus] | |
| 45 | FLJ90345 | sp\|Q8N196\|SIX5_HUMAN Homeobox | ref\|NP_787071.2\|sine oculis | ref\|XP_5914032\|PREDICTED: similar to sine ocuils | ref\|NP_035513.1\|sine oculis- | dbj\|BAA11824.1\|Six5 [Mus |
| | | protein SIX5 (DM locus-associated | homeobox homolog 5 [Homo | homeobox homolog 5 isoform 1 [Bos taurus] | related homeobox 5 homolog | musculus] |
| | | homeodomin protein) | sapiens] | | [Mus musculus]¥ | |
| | | | | | sp\|P70178\|SIX5_MOUSE | |
| | | | | | Homeobox protein | |
| | | | | | SIX5 (DM locus-associated | |
| | | | | | homeodomain protein | |
| | | | | | homolog) | |
| 46 | FLJ26550 | ref\|NP_006746.1\|transaldolase 1 [Homo | gb\|AAH18847.2\|TALDO1 | ref\|XP_533146.1\|PREDICTED: similar to | gb\|AAH59126.1\| | ref\|NP_035658.1\|transaldolase 1 |
| | | sapiens]¥ gb\|AAH10103.1\|Transaldolase | protein [Homo sapiens] | transaldolase 1 isoform 4 [Canis familiaris] | Transaldolase 1 [Rattus | [Mus |
| | | 1 [Homo sapiens]¥ gb\|AAF40478.1\| | | | norvegicus]¥ ref\|NP_113999.2\| | musculus]¥ gb\|AAH04754.1\| |
| | | transaldolase [Homo | | | transaldolase 1 [Rattus | Transaldolase 1 [Mus |
| | | sapiens]¥ gb\|AAB53943.1\|transaldolase | | | norvegicus] | musculus]¥ gb\|AAH94277.1\| |
| | | [Homo | | | | Transaldolase 1 [Mus |
| | | sapiens]¥ sp\|P37837\|TALDO_HUMAN | | | | musculus]¥ |
| | | Transaldolase¥ gb\|AAC52068.1\| | | | | sp\|Q93092\|TALDO_MOUSE |
| | | transaldolase-associated protein [Homo | | | | Transaldolase¥ gb\|AAB83955.1\| |
| | | sapiens]¥ pdb\|1F05\|B Chain B, Crystal | | | | transaldolase [Mus musculus] |
| | | Structure Of Human | | | | |
| | | Transaldolase¥ pdb\|1F05\|A Chain A, | | | | |
| | | Crystal Structure Of Human | | | | |
| | | Transaldolase | | | | |

TABLE 6-17

| | | | | |
|---|---|---|---|---|
| 47 | FLJ90015 | ref\|XP_517095.1\|PREDICTED: similar to | ref\|XP_861499.1\| | ref\|XP_600618.2\|PREDICTED: |
| | | protein associated with MRG, 14 kDa; T-cell | PREDICTED: similar to Mof4 | similar to Mof4 family associated |
| | | activation protein [Pan | family associated protein 1 | protein 1, partial [Bos taurus] |
| | | troglodytes]¥ ref\|NP_150638.1\|Mof4 family | isoform 2 [Canis familiaris]¥ | |
| | | associated protein 1 [Homo | ref\|XP_850453.1\| | |
| | | sapiens]¥ emb\|CAG33425.1\|PGR1 [Homo | PREDICTED: similar to Mof4 | |
| | | sapiens]¥ gb\|AAH22797.1\|Mof4 family | family associated protein 1 | |
| | | associated protein 1 [Homo | isoform 1 [Canis familiaris] | |
| | | sapiens]¥ gb\|AAD38498.1\|T-cell activation | | |
| | | protein [Homo sapiens] | | |
| 48 | FLJ39454 | ref\|NP_073745.2\|von Willebrand factor A | gb\|AAH03543.2\|VWA1 | ref\|XP_582281.2\|PREDICTED: |
| | | domain-associated protein isoform 1 [Homo | protein [Homo sapiens] | similar to von Willebrand factor |
| | | sapiens]¥ emb\|CAI22657.1\|von Willebrand | | A domain-associated protein |
| | | factor A domain-associated protein (WARP) | | isoform 1 [Bos taurus] |
| | | [Homo sapiens]¥ gb\|AAH59409.1\|Von | | |
| | | Willebrand factor A domain-associated | | |
| | | protein, isoform 1 [Homo sapiens] | | |
| 49 | FLJ45115 | sp\|Q96L91\|EP400_HUMAN E1A binding | ref\|NP_056224.2\|E1A | dbj\|BAB47447.1\|KIAA1818 |
| | | protein p400 (p400 kDa SWI2/SNF2- | binding protein p400 [Homo | protein [Homo sapiens] |
| | | associated protein) (Domino homolog) | sapiens] | |
| | | (hDomino) (CAG repeat protein 32) | | |
| | | (Trinucleotide repeat-containing gene 12 | | |
| | | protein) | | |
| 50 | FLJ90066 | gb\|AAH34732.1\|BM88 antigen [Homo | gb\|AAF60309.1\|BM88 | gb\|AAH23032.1\|BM88 antigen |
| | | sapiens]¥ gb\|AAP57306.1\|BM88 antigen | antigen [Homo sapiens] | [Mus musculus]¥ dbj\|BAC37512.1\| |
| | | [Homo sapiens]¥ ref\|NP_057648.2\|BM88 | | unnamed protein product [Mus |
| | | antigen [Homo sapiens]¥ dbj\|BAC11051.1\| | | musculus]¥ gb\|AAF62099.1\| |
| | | unnamed protein product [Homo | | BM88 antigen [Mus |
| | | sapiens]¥ sp\|Q8N111\|BM88_HUMAN BM88 | | musculus]¥ ref\|NP_067291.1\| |
| | | antigen | | BM88 antigen [Mus |
| | | | | musculus]¥ sp\|Q9JKC6\|BM88_MOUSE |
| | | | | BM88 antigen |

| | | |
|---|---|---|
| 47 | gb\|AAI02899.1\|Unknown | ref\|XP_526513.1\| |
| | (protein for MGC: 128271) | PREDICTED: similar to |
| | [Bos taurus] | PP784 [Pan troglodytes] |
| 48 | ref\|XP_848795.1\| | ref\|NP_954572.1\|von |
| | PREDICTED: similar to von | Willebrand factor A |
| | Willebrand factor A domain- | domain-associated protein |
| | associated protein isoform 1 | isoform 2 [Homo |
| | [Canis familiaris] | sapiens]¥ dbj\|BAB15264.1\| |
| | | unnamed protein product |
| | | [Homo sapiens] |
| 49 | gb\|AAK97789.1\|p400 | ref\|XP_878064.1\| |
| | SWI2/SNF2-associated | PREDICTED: similar to |
| | protein [Homo sapiens] | Domino isoform 4 [Bos taurus] |
| 50 | gb\|AAH89963.1\|BM88 | dbj\|BAB23812.1\|unnamed |
| | antigen [Rattus | protein product [Mus |
| | norvegicus¥ ref\|NP_001014185.1\| | musculus] |
| | BM88 antigen [Rattus | |
| | norvegicus¥ ref\|XP_341960.1\| | |
| | PREDICTED: similar to | |
| | BM88 antigen [Rattus | |
| | norvegicus] | |

TABLE 6-18

| | | | | | | |
|---|---|---|---|---|---|---|
| 51 | FLJ37995 | ref\|NP_940986.1\|carbonic | ref\|XP_574890.1\| | ref\|NP_078771.1\|carbonic anhydrase 13 [Mus | ref\|XP_544159.1\| | ref\|XP_222295.2\| |
| | | anhydrase XIII [Homo | PREDICTED: | musculus]¥ gb\|AAH64050.1\|Carbonic | PREDICTED: | PREDICTED: |
| | | sapiens]¥ gb\|AAH52602.1\| | similar to | anhydrase 13 [Mus musculus]¥ dbj\| | similar to | similar to |
| | | Carbonic anhydrase XIII | carbonic | BAE30845.1\| unnamed protein product [Mus | Carbonic | carbonic |
| | | [Homo | anhydrase 13 | musculus]¥ dbj\|BAE31705.1\| unnamed protein | anhydrase XIII | anhydrase 13 |
| | | sapiens]¥ dbj\|BAC04528.1\| | [Rattus | product [Mus musculus]¥ dbj\|BAE29942.1\| | (Carbonate | [Rattus |
| | | unnamed protein product | norvegicus] | unnamed protein product [Mus musculus]¥ dbj\| | dehydratase XIII) | norvegicus] |
| | | [Homo | | BAE29922.1\| unnamed protein product [Mus | (CA-XIII) | |
| | | sapiens]¥ | | musculus]¥ dbj\|BAE30468.1\|unnamed protein | [Canis familiaris] | |
| | | sp\|Q8N1Q1\| | | product [Mus musculus]¥ dbj\|BAE36996.1\| | | |
| | | CAH13_HUMAN | | unnamed protein product [Mus | | |
| | | Carbonic anhydrase XIII | | musculus]¥ dbj\|BAE31927.1\|unnamed protein | | |
| | | (Carbonate dehydratase | | product [Mus musculus]¥ dbj\|BAE31849.1\| | | |
| | | XIII) (CA-XIII) | | unnamed protein product [Mus | | |
| | | | | musculus]¥ dbj\|BAB26742.1\|unnamed protein | | |
| | | | | product [Mus musculus]¥ gb\|AAK16672.1\| | | |
| | | | | carbonic anhydrase XIII [Mus | | |
| | | | | musculus]¥ sp\|Q9D6N1\|CAH13_MOUSE | | |
| | | | | Carbonic anhydrase 13 (Carbonic anhydrase | | |
| | | | | XIII) (Carbonate dehydratase | | |
| | | | | XIII) (CA-XIII) | | |

TABLE 6-19

| | | | | |
|---|---|---|---|---|
| 52 | FLJ26058 | ref\|XP_574616.1\| | gb\|AAP36704.1\|Homo sapiens | ref\|NP_001395.1\|eukaryotic translation elongation factor 1 |
| | | PREDICTED: | eukaryotic translation | gamma [Homo sapiens]¥ gb\|AAH31012.1\|Eukaryotic |
| | | eukaryotic | elongation factor 1 gamma | translation elongation factor 1 gamma [Homo |
| | | translation | [synthetic | sapiens]¥ gb\|AAH28179.1\|Eukaryotic translation elongation |
| | | elongation factor | construct]¥ gb\|AAX43300.1\| | factor 1 gamma [Homo sapiens]¥ gb\|AAH15813.1\| |
| | | 1 gamma | eukaryotic translation | Eukaryotic translation elongation factor 1 gamma [Homo |
| | | [Rattus | elongation factor 1 gamma | sapiens]¥ gb\|AAH67738.1\|Eukaryotic translation elongation |
| | | norvegicus] | [synthetic | factor 1 gamma [Homo sapiens]¥ gb\|AAH06509.1\| |
| | | | construct]¥ gb\|AAX43299.1\| | Eukaryotic translation elongation factor 1 gamma [Homo |
| | | | eukaryotic translation | sapiens]¥ gb\|AAH00384.1\|Eukaryotic translation elongation |
| | | | elongation factor 1 gamma | factor 1 gamma [Homo sapiens]¥ gb\|AAP35323.1\| |
| | | | [synthetic construct] | eukaryotic translation elongation factor 1 gamma [Homo |
| | | | | sapiens]¥ emb\|CAG28553.1\|EEF1G [Homo |
| | | | | sapiens]¥ gb\|AAX41658.1\|eukaryotic translation elongation |
| | | | | factor 1 gamma [synthetic construct]¥ gb\|AAH09865.1\| |
| | | | | Eukaryotic translation elongation factor 1 gamma [Homo |
| | | | | sapiens]¥ gb\|AAH06520.1\|Eukaryatic translation elongation |
| | | | | factor 1 gamma [Homo sapiens]¥ sp\|P26641\|EF1G_HUMAN |
| | | | | Elongation factor 1-gamma (EF-1-gamma) (eEF-1B |
| | | | | gamma)¥ emb\|CAA45089.1\|homologue to elongation factor |
| | | | | 1-gamma from A. salina [Homo sapiens]¥ emb\|CAA77630.1\| |
| | | | | elongation factor-1-gamma [Homo sapiens] |

| | | |
|---|---|---|
| 52 | gb\|AAH13918.1\| | dbj\|BAE00947.1\| |
| | Eukaryotic | unnamed protein |
| | translation elongation factor 1 | product [Macaca fascicularis] |
| | gamma [Homo sapiens] | |

[Table 6-20]-[Table 6-25]

| | | | | | | |
|---|---|---|---|---|---|---|
| 53 | FLJ46369 | dbj\|BAC87345.1\| | ref\|XP_853907.1\| | ref\|XP_532471.2\| | ref\|XP_852186.1\|PREDICTED: similar to Nascent polypeptide-associated | ref\|XP_853049.1\| |
| | | unnamed protein | PREDICTED: | PREDICTED: | complex alpha subunit, muscle-specific form (Alpha-NAC, muscle-specific form), | PREDICTED: similar |
| | | product [Homo | similar to | similar to ZK84.1 | partial [Canis familiaris] | to adenylate kinase 3 |
| | | sapiens] | CG13648-PA | [Canis familiaris] | | [Canis familiaris] |
| | | | [Canis familiaris] | | | |
| 54 | FLJ16517 | ref\|NP_001004317.1\| | ref\|XP_539064.2\| | ref\|XP_345125.2\| | ref\|NP_001026942.1\|lin-28 homolog b [Mus musculus]¥ gb\|AAZ38894.1\|LIN28B | ref\|NP_001029990.1\| |
| | | hypothetical | PREDICTED: | PREDICTED: | [Mus musculus]¥ ref\|XP_354572.2\|PREDICTED: similar to FLJ16517 protein | lin-28 homolog B |
| | | protein | similar to RNA- | similar to | [Mus musculus] | [Gallus |
| | | LOC389421 | binding protein | FLJ16517 protein | | gallus]¥ gb\|AAZ33896.1\| |
| | | [Homo | LIN-28 [Canis | [Rattus | | LIN28B [Gallus |
| | | sapiens]¥ gb\|AAZ38897.1\| | familiaris] | norvegicus] | | gallus] |
| | | LIN28B | | | | |
| | | [Homo | | | | |
| | | sapiens]¥ dbj\|BAD18558.1\| | | | | |
| | | unnamed | | | | |
| | | protein product | | | | |
| | | [Homo sapiens] | | | | |
| 55 | FLJ26591 | ref\|XP_519076.1\| | ref\|XP_507684.1\| | ref\|XP_531396.1\| | gb\|AAI06031.1\|Unknown (protein for MGC: 117158) [Homo | emb\|CAG32988.1\| |
| | | PREDICTED: | PREDICTED: | PREDICTED: | sapiens]¥ ref\|NP_086953.1\|peptidylprolyl isomerase A isoform 1 [Homo | PPIA [Homo |
| | | similar to | similar to | similar to | sapiens]¥ gb\|AAU13906.1\|peptidylprolyl isomerase A (cyclophilin A) [Homo | sapiens]¥ ref\|NP_001008741.1\| |
| | | peptidylprolyl | peptidylprolyl | peptidylprolyl | sapiens]¥ ref\|NP_001027981.1\|cyclophilin A [Macaca mulatta]¥ gb\|AAH73992.1\| | peptidylprolyl |
| | | isomerase A | isomerase A | isomerase A | Peptidylprolyl isomerase A, isoform 1 [Homo sapiens]¥ gb\|AAH13915.1\| | isomerase A-like |
| | | isoform 1; | isoform 1; | isoform 1; | Peptidylprolyl isomerase A, isoform 1 [Homo sapiens]¥ gb\|AAH00689.1\| | [Homo sapiens] |
| | | cyclophilin A; | cyclophilin A; | cyclophilin A; | Peptidylprolyl isomerase A, isoform 1 [Homo sapiens]¥ gb\|AAH03026.2\| | |
| | | peptidyl-prolyl cis- | peptidyl-prolyl cis- | peptidyl-prolyl cis- | Peptidylprolyl isomerase A, isoform 1 [Homo sapiens]¥ gb\|AAH05320.1\| | |
| | | trans isomerase A; | trans isomerase A; | trans isomerase A; | Peptidylprolyl isomerase A, isoform 1 [Homo sapiens]¥ pdb\|1YND\|B Chain B, | |
| | | T cell cyclophilin; | T cell cyclophilin; | T cell cyclophilin; | Structure Of Human Cyclophilin A In Complex With The Novel | |
| | | rotamase; | rotamase; | rotamase; | Immunosuppressant Sanglifehrin A At 1.6a Resolution¥ pdb\|1YND\|A Chain A, | |
| | | cyclosporin A- | cyclosporin A- | cyclosporin A- | Structure Of Human Cyclophilin A In Complex With The Novel | |
| | | binding protein | binding protein | binding protein | Immunosuppressant Sanglifehrin A At 1.6a Resolution¥ sp\|P62937\|PPIA_HUMAN | |
| | | [Pan troglodytes] | [Pan troglodytes] | [Pan troglodytes] | Peptidyl-prolyl cis-trans isomerase A (PPIase) (Rotamase) (Cyclophilin A) | |
| | | | | | (Cyclosporin A-binding protein)¥ pdb\|1CWM\|A Chain A, Human Cyclophilin A | |
| | | | | | Complexed With 4 Meile Cyclosporin¥ gb\|AAB81961.1\|cyclophilin A [Macaca | |
| | | | | | mulatta]¥ gb\|AAB81960.1\|cyclophilin A [Cercopithecus | |
| | | | | | aethiops]¥ gb\|AAB81959.1\|cyclophilin A [Papio hamadryas]¥ emb\|CAA68264.1\| | |
| | | | | | unnamed protein product [Homo sapiens]¥ emb\|CAA37039.1\|peptidylpropyl | |
| | | | | | isomerase [Homo sapiens]¥ pdb\|1W8V\|A Chain A, Enzymatic And Structural | |
| | | | | | Characterization Of Non Peptide Ligand Cyclophilin Complexes¥ pdb\|1W8M\|A | |
| | | | | | Chain A, Enzymatic And Structural Characterisation Of Non Peptide Ligand | |
| | | | | | Cyclophilin Complexes¥ pdb\|1W8L\|A Chain A, Enzymatic And Structural | |
| | | | | | Characterization Of Non Peptide Ligand Cyclophilin | |
| | | | | | Complexes¥ sp\|P62941\|PPIA_PAPAN Peptidyl-prolyl cis-trans isomerase A | |
| | | | | | (PPIase) (Rotamase) (Cyclophilin A) (Cyclosporin A-binding | |
| | | | | | protein)¥ sp\|P62940\|PPIA_MACMU Peptidyl-prolyl cis-trans isomerase A | |
| | | | | | (PPIase) (Rotamase) (Cyclophilin A) (Cyclosporin A-binding | |
| | | | | | protein)¥ sp\|P62938\|PPIA_CERAE Peptidyl-prolyl cis-trans isomerase A (PPIase) | |
| | | | | | (Rotamase) (Cyclophilin A) (Cyclosporin A-binding protein)¥ pdb\|1MIK\|A Chain A, | |
| | | | | | The Role Of Water Molecules In The Structure-Based Design Of (5- | |
| | | | | | Hydroxynorvaline)-2-Cyclosporin: Synthesis, Biological Activity, And | |
| | | | | | Crystallographic Analysis With Cyclophilin A¥ pdb\|1NMK\|B Chain B, The | |
| | | | | | Sanglifehrin-Cyclophilin Interaction: Degradation Work, Synthetic Macrocyclic | |
| | | | | | Analogues, X-Ray Crystal Structure And Binding Data¥ pdb\|1NMK\|A Chain A, | |
| | | | | | The Sanglifehrin-Cyclophilin Interaction: Degradation Work, Synthetic Macrocyclic | |
| | | | | | Analogues, X-Ray Crystal Structure And Binding Data¥ pdb\|1M9Y\|F Chain F, X- | |
| | | | | | Ray Crystal Structure Of Cyclophilin AHIV-1 Ca N-Terminal Domain (1-146) | |
| | | | | | M-Type H87a, G89a Complex.¥ pdb\|1M9Y\|E Chain E, X-Ray Crystal Structure Of | |
| | | | | | Cyclophilin AHIV-1 Ca N-Terminal Domain (1-146) M-Type H87a, G89a | |
| | | | | | Complex.¥ pdb\|1M9Y\|B Chain B, X-Ray Crystal Structure Of Cyclophilin AHIV-1 | |
| | | | | | Ca N-Terminal Domain (1-146) M-Type H87a, G89a Complex.¥ pdb\|1M9Y\|A | |
| | | | | | Chain A, X-Ray Crystal Structure Of Cyclophilin AHIV-1 Ca N-Terminal | |
| | | | | | Domain (1-146) M-Type H87a, G89a Complex.¥ pdb\|1M9X\|F Chain F, X-Ray | |
| | | | | | Crystal Structure Of Cyclophilin AHIV-1 Ca N-Terminal Domain (1-146) M- | |
| | | | | | Type H87a, A88m, G89a Complex.¥ pdb\|1M9X\|E Chain E, X-Ray Crystal Structure | |
| | | | | | Of Cyclophilin AHIV-1 Ca N-Terminal Domain (1-146) M-Type | |
| | | | | | H87a, A88m, G89a Complex.¥ pdb\|1M9X\|B Chain B, X-Ray Crystal Structure Of | |
| | | | | | Cyclophilin AHIV-1 Ca N-Terminal Domain (1-146) M-Type H87a, A88m, G89a | |
| | | | | | Complex.¥ pdb\|1M9X\|A Chain A, X-Ray Crystal Structure Of Cyclophilh AHIV-1 | |
| | | | | | Ca N-Terminal Domain (1-146) M-Type H87a, A88m, G89a | |
| | | | | | Complex.¥ pdb\|1M9F\|B Chain B, X-Ray Crystal Structure Of Cyclophilin AHIV-1 | |
| | | | | | Ca N-Terminal Domain (1-146) M-Type H87a, A88m Complex.¥ pdb\|1M9F\|A | |
| | | | | | Chain A, X-Ray Crystal Structure Of Cyclophilin AHIV-1 Ca N-Terminal | |
| | | | | | Domain (1-146) M-Type H87a, A88m Complex.¥ pdb\|1M9D\|B Chain B, X-Ray | |
| | | | | | Crystal Structure Of Cyclophilin AHIV-1 Ca N-Terminal Domain (1-146) O- | |
| | | | | | Type Chimera Complex.¥ pdb\|M9D\|A Chain A, X-Ray Crystal Structure Of | |
| | | | | | Cyclophilin AHIV-1 Ca N-Terminal Domain (1-146) O-Type Chimera | |
| | | | | | Complex.¥ pdb\|1M9C\|B Chain B, X-Ray Crystal Structure Of Cyclophilin AHIV-1 | |
| | | | | | Ca N-Terminal Domain (1-146) M-Type Complex.¥ pdb\|1M9C\|A Chain A, X- | |
| | | | | | Ray Crystal Structure Of Cyclophilin AHIV-1 Ca N-Terminal Domain (1-146) | |
| | | | | | M-Type Complex.¥ pdb\|1MF8\|C Chain C, Crystal Structure Of Human | |
| | | | | | Calcineurin Complexed With Cyclosporin A And Human Cyclophilin¥ pdb\|1M63\|G | |
| | | | | | Chain G, Crystal Structure Of Calcineurin-Cyclophilin-Cyclosporin Shows | |
| | | | | | Common But Distinct Recognition Of Immunophilin-Drug | |
| | | | | | Complexes¥ pdb\|1M63\|C Chain C, Crystal Structure Of Calcineurin-Cyclophilin- | |
| | | | | | Cyclosporin Shows Common But Distinct Recognition Of Immunophilin-Drug | |
| | | | | | Complexes¥ pdb\|1VBT\|B Chain B, Structure Of Cyclophilin Complexed With | |
| | | | | | Sulfur-Substituted Tetrapeptide Aapf¥ pdb\|1VBT\|A Chain A, Structure Of | |
| | | | | | Cyclophilin Complexed With Sulfur-Substituted Tetrapeptide Aapf¥ pdb\|1VBS\|A | |
| | | | | | Chain A, Structure Of Cyclophilin Complexed With (D)ala Containing | |
| | | | | | Tetrepeptide¥ pdb\|1OCA\|Human Cyclophilin A, Unligated, Nmr, 20 | |
| | | | | | Structures¥ pdb\|1FGL\|A Chain A, Cyclophilin A Complexed With A Fragment Of | |
| | | | | | Hiv-1 Gag Protein¥ pdb\|1CWL\|A Chain A, Human Cyclophilin A Complexed With 4 | |
| | | | | | 4-Hydroxy-Meleu Cyclosporin¥ pdb\|1CWK\|A Chain A, Human Cyclophilin A | |
| | | | | | Complexed With 1-(6,7-Dihydro)mebmt 2-Val 3-D-(2-S-Methy)sarcosine | |
| | | | | | Cyclosporin¥ pdb\|1CWJ\|A Chain A, Human Cyclophilin A Complexed With 2-Val | |
| | | | | | 3-S-Methyl-Sarcosine Cyclosporin¥ pdb\|1CWI\|A Chain A, Human Cyclophilin A | |
| | | | | | Complexed With 2-Val 3-(N-Methyl)-D-Alanine Cyclosporin¥ pdb\|1CWH\|A Chain | |
| | | | | | A, Human Cyclophilin A Complexed With 3-D-Ser Cyclosporin¥ pdb\|1CWF\|A | |
| | | | | | Chain A, Human Cyclophilin A Complexed With 2-Val Cyclosporin¥ pdb\|1AK4\|B | |
| | | | | | Chain B, Human Cyclophilin A Bound To The Amino-Terminal Domain Of Hiv-1 | |
| | | | | | Capsid¥ pdb\|1AK4\|A Chain A, Human Cyclophilin A Bound To The Amino- | |
| | | | | | Terminal Domain Of Hiv-1 Capsid¥ pdb\|2RMB\|S Chain S, Cyclophilin A | |
| | | | | | (E.C.5.2.1.8) Complexed With Dimethyl-Cyclosporin A¥ pdb\|2RMB\|Q Chain Q, | |
| | | | | | Cyclophilin A (E.C.5.2.1.8) Complexed With Dimethyl-Cyclosporin A¥ pdb\|2RMB\|O | |
| | | | | | Chain O, Cyclophilin A (E.C.5.2.1.8) Complexed With Dimethyl-Cyclosporin | |
| | | | | | A¥ pdb\|2RMB\|M Chain M, Cyclophilin A (E.C.5.2.1.8) Complexed With Dimethyl- | |
| | | | | | Cyclosporin A¥ pdb\|2RMB\|K Chain K, Cyclophilin A (E.C.5.2.1.8) Complexed With | |
| | | | | | Dimethyl-Cyclosporin A¥ pdb\|2RMB\|I Chain I, Cyclophilin A (E.C.5.2.1.8) | |
| | | | | | Complexed With Dimethyl-Cyclosporin A¥ pdb\|2RMB\|G Chain G, Cyclophilin A | |
| | | | | | (E.C.5.2.1.8) Complexed With Dimethyl-Cyclosporin A¥ pdb\|2RMB\|E Chain E, | |
| | | | | | Cyclophilin A (E.C.5.2.1.8) Complexed With Dimethyl-Cyclosporin A¥ pdb\|2RMB\|C | |
| | | | | | Chain C, Cyclophilin A (E.C.5.2.1.8) Complexed With Dimethyl-Cyclosporin | |
| | | | | | A¥ pdb\|2RMB\|A Chain A, Cyclophilin A (E.C.5.2.1.8) Complexed With Dimethyl- | |
| | | | | | Cyclosporin A¥ pdb\|2RMA\|S Chain S, Cyclophilin A (E.C.5.2.1.8) Complexed With | |
| | | | | | Cyclosporin A¥ pdb\|2RMA\|Q Chain Q, Cyclophilin A (E.C.5.2.1.8) Complexed With | |
| | | | | | Cyclosporin A¥ pdb\|2RMA\|O Chain O, Cyclophilin A (E.C.5.2.1.8) Complexed With | |
| | | | | | Cyclosporin A¥ pdb\|2RMA\|M Chain M, Cyclophilin A (E.C.5.2.1.8) Complexed With | |
| | | | | | Cyclosporin A¥ pdb\|2RMA\|K Chain K, Cyclophilin A (E.C.5.2.1.8) Complexed With | |
| | | | | | Cyclosporin A¥ pdb\|2RMA\|I Chain I, Cyclophilin A (E.C.5.2.1.8) Complexed With | |
| | | | | | Cyclosporin A¥ pdb\|2RMA\|G Chain G, Cyclophilin A (E.C.5.2.1.8) Complexed With | |
| | | | | | Cyclosporin A¥ pdb\|2RMA\|E Chain E, Cyclophilin A (E.C.5.2.1.8) Complexed With | |
| | | | | | Cyclosporin A¥ pdb\|2RMA\|C Chain C, Cyclophilin A (E.C.5.2.1.8) Complexed With | |
| | | | | | Cyclosporin A¥ pdb\|2RMA\|A Chain A, Cyclophilin A (E.C.5.2.1.8) Complexed With | |
| | | | | | Cyclosporin A¥ pdb\|2CPL\|Cyclophilin A¥ pdb\|1CWC\|A Chain A, Mol_id: 1; | |
| | | | | | Molecule: Cyclophilin A; Chain: A; Engineered: Yes; Mol_id: 2; Molecule: [4,N- | |
| | | | | | Dimethylnorleucine]4-Cyclosporin; Chain: C; Engineered: Yes¥ pdb\|1CWB\|A Chain | |
| | | | | | A, Mol_id: 1; Molecule: Cyclophilin A; Chain: A; Engineered: Yes; Mol_id: 2; Molecule: | |
| | | | | | [4-[(E)-2-Butenyl]-4,4,N-Trimethyl-L-Threonine]1-Cyclosporin; Chain: C; | |
| | | | | | Engineered: Yes¥ pdb\|1CWA\|A Chain A, Mol_id: 1; Molecule: Cyclophilin A; Chain: | |
| | | | | | A; Engineered: Yes; Mol_id: 2; Molecule: Cyclosporin A; Chain: C; Engineered: Yes | |

TABLE 6-26

| | | | |
|---|---|---|---|
| 56 | FLJ26596 | ref\|XP_527283.1\| | gb\|AAI04199.1\|H2A histone family, member D [Homo |
| | | PREDICTED: | sapiens]¥ gb\|AAI04200.1\|H2A histone family, member D [Homo |
| | | similar to | sapiens]¥ ref\|XP_876240.1\|PREDICTED: similar to Histone H2A.1 |
| | | Hist2h2aa1 | [Bos taurus]¥ ref\|XP_873767.1\|PREDICTED: similar to Histone H2A.1 |
| | | protein [Pan | [Bos taurus]¥ ref\|XP_874094.1\|PREDICTED: similar to Histone H2A.1 |
| | | troglodytes] | [Bos taurus]¥ ref\|XP_607721.2\|PREDICTED: similar to Histone H2A.1 |
| | | | [Bos taurus]¥ ref\|XP_873992.1\|PREDICTED: similar to Histone H2A.1 |
| | | | [Bos taurus]¥ ref\|NP_066408.1\|H2A histone family, member P [Homo |
| | | | sapiens]¥ ref\|NP_003505.1\|H2A histone family, member N [Homo |
| | | | sapiens]¥ ref\|NP_003502.1\|H2A histone family, member I [Homo |
| | | | sapiens]¥ ref\|NP_003501.1\|H2A histone family, member D [Homo |
| | | | sapiens]¥ ref\|NP_003500.1\|H2A histone family, member C [Homo |
| | | | sapiens]¥ ref\|XP_545419.1\|PREDICTED: similar to Histone H2A.1 |
| | | | [Canis familiaris]¥ gb\|AAH69306.1\|H2A histone family, member I |
| | | | [Homo sapiens]¥ gb\|AAH16677.1\|H2A histone family, member P |
| | | | [Homo sapiens]¥ emb\|CAD24077.1\|histone 1, H2am [Homo |
| | | | sapiens]¥ emb\|CAD24073.1\|histone 1, H2al [Homo |
| | | | sapiens]¥ emb\|CAB11417.1\|histone 1, H2ak [Homo |
| | | | sapiens]¥ emb\|CAA16948.1\|RP1-86C11.5 [Homo |
| | | | sapiens]¥ emb\|CAA15669.1\|histone 1, H2ai [Homo |
| | | | sapiens]¥ emb\|CAB06037.1\|histone H2A [Homo |
| | | | sapiens]¥ emb\|CAB06034.1\|histone H2A [Homo |
| | | | sapiens]¥ emb\|CAA58539.1\|histone H2A [Homo |
| | | | sapiens]¥ emb\|CAA40417.1\|histone H2A.1 [Homo |
| | | | sapiens]¥ gb\|AAN59974.1\|histone H2A [Homo |
| | | | sapiens]¥ gb\|AAN59973.1\|histone H2A [Homo |
| | | | sapiens]¥ gb\|AAN59972.1\|histone H2A [Homo |
| | | | sapiens]¥ gb\|AAN59970.1\|histone H2A [Homo |
| | | | sapiens]¥ gb\|AAN59968.1\|histone H2A [Homo |
| | | | sapiens]¥ gb\|AAX36557.1\|histone 1 H2ak [synthetic |
| | | | construct]¥ gb\|AAH71668.1\|H2A histone family, member N [Homo |
| | | | sapiens]¥ gb\|AAH32756.1\|H2A histone family, member N [Homo |
| | | | sapiens]¥ sp\|P02261\|H2AC_HUMAN Histone H2A.c/d/i/n/p (H2A.1) |
| | | | (H2A/c) (H2A/d) (H2A/i) (H2A/n) (H2A/p) (H2A.1b)¥ gb\|AAC24466.1\| |
| | | | histone H2A.1b [Homo sapiens] |

| | | | |
|---|---|---|---|
| 56 | prf\|\|1109175A | ref\|XP_602496.2\| | ref\|XP_527272.1\| |
| | homeostatic thymus | PREDICTED: similar to | PREDICTED: similar to |
| | hormone alpha | Histone H2A.1 [Bos taurus] | Histone H2A.1 [Pan |
| | | | troglodytes] |

TABLE 6-27

| | | | | |
|---|---|---|---|---|
| 57 | FLJ90480 | dbj\|BAC11317.1\|unnamed protein product | ref\|NP_852149.1\|zinc finger, CCCH-type | dbj\|BAB47476.2\| |
| | | [Homo sapiens] | with G patch domain isoform b [Homo | KIAA1847 protein |
| | | | sapiens] | [Homo sapiens] |
| 58 | FLJ43067 | ref\|NP_002620.1\|phosphoglycerate mutase | pdb\|1YJX\|L Chain L, Crystal Structure Of | dbj\|BAD96816.1\| |
| | | 1 (brain) [Homo sapiens]¥ gb\|AAH53356.1\| | Human B Type Phosphoglycerate | phosphoglycerate |
| | | Phosphoglycerate mutase 1 (brain) [Homo | Mutase¥ pdb\|1YJX\|K Chain K, Crystal | mutase 1 (brain) |
| | | sapiens]¥ gb\|AAH73742.1\| | Structure Of Human B Type | variant [Homo |
| | | Phosphoglycerate mutase 1 (brain) [Homo | Phosphoglycerate Mutase¥ pdb\|1YJX\|J | sapiens] |
| | | sapiens]¥ emb\|CAI40778.1\| | Chain J, Crystal Structure Of Human B | |
| | | phosphoglycerate mutase 1 (brain) [Homo | Type Phosphoglycerate | |
| | | sapiens]¥ gb\|AAH66959.1\| | Mutase¥ pdb\|1YJX\|I Chain I, Crystal | |
| | | Phosphoglycerate mutase 1 (brain) [Homo | Structure Of Human B Type | |
| | | sapiens]¥ gb\|AAH10038.1\| | Phosphoglycerate Mutase¥ pdb\|1YJX\|H | |
| | | Phosphoglycerate mutase 1 (brain) [Homo | Chain H, Crystal Structure Of Human B | |
| | | sapiens]¥ dbj\|BAE01661.1\|unnamed protein | Type Phosphoglycerate | |
| | | product [Macaca | Mutase¥ pdb\|1YJX\|G Chain G, Crystal | |
| | | fascicularis]¥ emb\|CAG46460.1\|PGAM1 | Structure Of Human B Type | |
| | | [Homo sapiens]¥ gb\|AAG01990.1\|similar to | Phosphoglycerate Mutase¥ pdb\|1YJX\|F | |
| | | Homo sapiens phosphoglycerate mutase | Chain F, Crystal Structure Of Human B | |
| | | (PGAM-B) mRNA with GenBank Accession | Type Phosphoglycerate | |
| | | Number J04173.1¥ gb\|AAH11678.1\| | Mutase¥ pdb\|1YJX\|E Chain E, Crystal | |
| | | Phosphoglycerate mutase 1 (brain) [Homo | Structure Of Human B Type | |
| | | sapiens]¥ sp\|P18669\|PGAM1_HUMAN | Phosphoglycerate Mutase¥ pdb\|1YJX\|D | |
| | | Phosphoglycerate mutase 1 | Chain D, Crystal Structure Of Human B | |
| | | (Phosphoglycerate mutase isozyme B) | Type Phosphoglycerate | |
| | | (PGAM-B) (BPG-dependent PGAM | Mutase¥ pdb\|1YJX\|C Chain C, Crystal | |
| | | 1)¥ gb\|AAA60071.1\|phosphoglycerate | Structure Of Human B Type | |
| | | mutase 2 | Phosphoglycerate Mutase¥ pdb\|1YJX\|B | |
| | | | Chain B, Crystal Structure Of Human B | |
| | | | Type Phosphoglycerate | |
| | | | Mutase¥ pdb\|1YJX\|A Chain A, Crystal | |
| | | | Structure Of Human B Type | |
| | | | Phosphoglycerate Mutase¥ pdb\|1YFK\|B | |
| | | | Chain B, Crystal Structure Of Human B | |
| | | | Type Phosphoglycerate | |
| | | | Mutase¥ pdb\|1YFK\|A Chain A, Crystal | |
| | | | Structure Of Human B Type | |
| | | | Phosphoglycerate Mutase | |
| 59 | FLJ25460 | dbj\|BAB71708.1\|unnamed protein product | ref\|XP_524039.1\|PREDICTED: ATPase, | dbj\|BAB63028.1\| |
| | | [Homo sapiens] | Class I, type 8B, member 3 [Pan | hypothetical |
| | | | troglodytes] | protein [Macaca |

| | | | |
|---|---|---|---|
| 57 | FLJ90480 | ref\|NP_115916.2\|zinc | ref\|NP_852150.1\|zinc finger, |
| | | finger, CCCH-type with G patch | CCCH-type with G patch |
| | | domain isoform a | domain isoform c [Homo |
| | | [Homo | sapiens]¥ gb\|AAH32612.1\|Zinc |
| | | sapiens]¥ sp\|Q8N5A5\|ZG | finger, CCCH-type with G patch |
| | | PAT_HUMAN Zinc finger | domain, isoform c [Homo |
| | | CCCH-type with G patch | sapiens] |
| | | domain protein (Zinc finger | |
| | | CCCH-type domain | |
| | | containing protein 9) | |
| 58 | FLJ43067 | ref\|XP_534980.1\| | gb\|AAI06140.1\|Unknown |
| | | PREDICTED: similar to | (protein for MGC: 118049) [Mus |
| | | Phosphoglycerate mutase | musculus]¥ gb\|AAH83090.1\| |
| | | 1 (Phosphoglycerate | Pgam1 protein [Mus |
| | | mutase isozyme B) | musculus]¥ gb\|AAH66844.1\| |
| | | (PGAM-B) (BPG- | Pgam1 protein [Mus |
| | | dependent PGAM 1) | musculus]¥ gb\|AAH65582.1\| |
| | | isoform 1 [Canis familiaris] | Pgam1 protein [Rattus |
| | | | norvegicus]¥ gb\|AAH02241.1\| |
| | | | Pgam1 protein [Mus |
| | | | musculus]¥ gb\|AAH05661.1\| |
| | | | Pgam1 protein [Mus |
| | | | musculus]¥ dbj\|BAE29794.1\| |
| | | | unnamed protein product [Mus |
| | | | musculus]¥ dbj\|BAE34975.1\| |
| | | | unnamed protein product [Mus |
| | | | musculus]¥ dbj\|BAE31223.1\| |
| | | | unnamed protein product [Mus |
| | | | musculus]¥ dbj\|BAE40755.1\| |
| | | | unnamed protein product [Mus |
| | | | musculus]¥ dbj\|BAE31802.1\| |
| | | | unnamed protein product [Mus |
| | | | musculus]¥ dbj\|BAE38168.1\| |
| | | | unnamed protein product [Mus |
| | | | musculus]¥ dbj\|BAB23672.1\| |
| | | | unnamed protein product [Mus |
| | | | musculus]¥ ref\|XP_619872.1\| |
| | | | PREDICTED: similar to |
| | | | phosphoglycerate mutase (EC |
| | | | 5.4.2.1) B chain-rat [Mus |
| | | | musculus]¥ |
| | | | sp\|Q9DBJ1\|PGAM1_MOUSE |
| | | | Phosphoglycerate |
| | | | mutase 1 (Phosphoglycerate |
| | | | mutase isozyme B) (PGAM-B) |
| | | | (BPG-dependent PGAM |
| | | | 1)¥ pir\|\|JC1132 |
| | | | phosphoglycerate mutase (EC |
| | | | 5.4.2.1) B chain-rat |
| 59 | FLJ25460 | ref\|XP_875482.1\| | dbj\|BAB71492.1\|unnamed |
| | | PREDICTED: similar to | protein product [Homo sapiens] |
| | | Potential phospholipid- | |
| | | transporting ATPase IK | |
| | | (ATPase class I type 8B | |
| | | member 3) [Bos taurus] | |

TABLE 6-28

| | | | |
|---|---|---|---|
| 60 | FLJ26806 | ref\|XP_496622.1\| | dbj\|BAC85324.1\|unnamed protein product [Homo |
| | | PREDICTED: FLJ40411 | sapiens] |
| | | protein [Homo sapiens] | |
| 61 | FLJ43911 | ref\|NP_001028258.1\| | ref\|NP_001028259.1\|hypothetical protein |
| | | hypothetical protein | LOC140733 isoform 2 [Homo |
| | | LOC140733 isoform 1 | sapiens]¥ emb\|CAI18920.1\|RP11-189J1.1 [Homo |
| | | [Homo sapiens] | sapiens]¥ emb\|CAH71089.1\|RP11-189J1.1 |
| | | | [Homo sapiens]¥ emb\|CAI22982.1\|RP11-189J1.1 |
| | | | [Homo sapiens] |
| 62 | FLJ44715 | gb\|AAI00995.1\|FUT11 | gb\|AAI00998.1\|Fucosyltransferase 11 (alpha (1,3) |
| | | protein [Homo | fucosyltransferase) [Homo |
| | | sapiens]¥ gb\|AAI00996.1\| | sapiens]¥ gb\|AAI00997.1\|Fucosyltransferase 11 |
| | | FUT11 protein [Homo | (alpha (1,3) fucosyltransferase) [Homo sapiens] |
| | | sapiens] | |
| 63 | FLJ90031 | ref\|XP_870413.1\| | ref\|NP_036364.2\|polymerase I and transcript |
| | | PREDICTED: similar to | release factor [Homo sapiens]¥ gb\|AAH66123.1\| |
| | | Polymerase I and | Polymerase I and transcript release factor [Homo |
| | | transcript release factor | sapiens]¥ sp\|Q6NZI2\|PTRF_HUMAN Polymerase I |
| | | [Bos taurus] | and transcript release factor (PTRF protein) |

| | | | |
|---|---|---|---|
| 60 | dbj\|BAE01189.1\|unnamed | dbj\|BAE21615.1\| | ref\|XP_526074.1\|PREDICTED: |
| | protein product [Macaca | unnamed protein | hypothetical protein XP_526074 |
| | fascicularis] | product [Mus | [Pan troglodytes] |
| | | musculus] | |
| 61 | ref\|XP_485071.2\|PREDICTED: | ref\|XP_578169.1\| | dbj\|BAE22244.1\|unnamed |
| | similar to CG5965-PA [Mus | PREDICTED: | protein product [Mus musculus] |
| | musculus] | hypothetical protein | |
| | | XP_578169 [Rattus | |
| | | norvegicus] | |
| 62 | ref\|NP_775811.1\| | ref\|XP_586457.2\| | dbj\|BAA07558.2\|KIAA0079 |
| | fucosyltransferase 11 (alpha | PREDICTED: similar | [Homo sapiens] |
| | (1,3) fucosyltransferase) | to fucosyltransferase | |
| | [Homo | 11 (alpha (1,3) | |
| | sapiens]¥ gb\|AAH36037.1\| | fucosyltransferase) | |
| | Fucosyltransferase 11 (alpha | [Bos taurus] | |
| | (1,3) fucosyltransferase) | | |
| | [Homo sapiens] | | |
| 63 | gb\|AAG27093.1\|leucine- | gb\|AAH73759.1\|PTRF | ref\|XP_548089.2\|PREDICTED: |
| | zipper protein FKSG13 [Homo | protein [Homo | similar to polymerase I and |
| | sapiens] | sapiens] | transcript release factor [Canis |
| | | | familiaris] |

### Homology Analysis 2 by BLASTX

The calculation program used was blastall 2.2.6. The target databases used were swiss-prot: 196277 (2005.10.25), (Refseq)hs: 24139 (2005.09.15), (Refseq)mouse: 18457 (2005.09.15), and (Refseq)rat: 9252 (2005.09.15). The cutoff value was established at 1.00E-05. The following data were processed by filtering:

### For Swiss-prot:

Having a definition beginning with “ALU SUBFAMILY”
Having a definition beginning with “Alu subfamily”
Having a definition beginning with “!!!! ALU SUBFAMILY”
Having a definition beginning with “B-CELL GROWTH FACTOR PRECURSOR”
Having a definition including “NRK2”
Having a definition beginning with “PROLINE-RICH”
Having a definition beginning with “GLYCINE-RICH”
Having a definition beginning with “EXTENSIN PRECURSOR”
Having a definition beginning with “COLLAGEN”
Having a definition beginning with “100 KD”
Having a definition beginning with “RETROVIRUS-RELATED POL POLYPROTEIN”
Having a definition beginning with “CUTICLE COLLAGEN”
Having a definition beginning with “HYPOTHETICAL”
Having a definition beginning with “Hypothetical”
Having a definition beginning with “SALIVARY PROLINE-RICH PROTEIN”
Having a definition beginning with “IMMEDIATE-EARLY PROTEIN”
Having the accession No “P49646”

### For Ref-seq:

Having a definition beginning with “hypothetical protein FLJ”
Having a definition beginning with “KIAA”
Having a definition beginning with “hypothetical protein DKFZ”
Having a definition beginning with “DKFZ”
Having a definition beginning with “RIKEN cDNA”
Having a definition beginning with “hypothetical protein MGC”
Having a definition beginning with “hypothetical protein”
Having a definition beginning with “hypothetical protein PP”
Having a definition beginning with “neuronal thread protein”
Having a definition beginning with “clone FLB”
Having a definition beginning with “hypothetical protein PRO”
Having a definition as “PRO0483 protein”
Having a definition including “MNC”
Having a definition including “MOST-1”
Having a definition beginning with “similar to”
Having a definition including “TPR gene on Y”
Having a definition beginning with “HSPC”
Having a definition beginning with “CGI-”
ReFSeq sequence composed of self only (information referenced from LL_tmpl)

The annotation information obtained by this analysis is shown in Tables 7-1 to 7-8.

TABLE 7-1

| | SEQ | | | SwissProt(BLASTP) | | |
|---|---|---|---|---|---|---|
| | ID | RefSeq(BLASTP) | | accession | | |
| FLJ No. | NO: | accession No. | definition | No. | definition | keyword |
| FLJ21182 | 1 | NP_004359.1 | calponin 2 | Q99439 | Calponin-2 (Calponin H2, smooth | Actin-binding; Calmodulin-binding; Direct |
| | | | isoform a | | muscle)(Neutral calponin) | protein sequencing; Multigene family; |
| | | | [Homo sapiens] | | | Repeat. |
| FLJ38597 | 2 | NP_599032.1 | smoothelin | P53814 | Smoothelin | Alternative splicing; Phosphorylation; |
| | | | isofrom a | | | Structural protein. |
| | | | [Homo sapiens] | | | |
| FLJ13700 | 3 | NP_003119.1 | spectrin, beta, | Q01082 | Spectrin beta chain, brain 1 | 3D-structure; Actin capping; Actin-binding; |
| | | | non-erythrocytic | | (Spectrin, non-erythroid beta chain | Alternative splicing; Calmodulin-binding; |
| | | | 1 isoform 1 | | 1) (Beta-II spectrin) (Fodrinbeta | Cytoskeleton; Membrane; Phosphorylation; |
| | | | [Homo sapiens] | | chain) | Repeat. |
| FLJ50683 | 4 | NP_005023.2 | plastin 3 | P13797 | T-plastin (Plastin-3) | 3D-structure; Actin-binding; Calcium; Direct |
| | | | [Homo sapiens] | | | protein sequencing; Phosphorylation; Repeat. |
| FLJ50199 | 5 | NP_004831.1 | Rac/Cdc42 | Q15052 | Rho guanine nucleotide exchange | 3D-structure; Alternative splicing; Guanine- |
| | | | guanine nucleotide | | factor 6(Rac/Cdc42 guanine | nucleotide releasing factor; Phosphorylation; |
| | | | exchange factor 6 | | nucleotide exchange factor | SH3 domain. |
| | | | [Homo sapiens] | | 6)(PAK-interacting exchange | |
| | | | | | factor alpha) (Alpha- | |
| | | | | | Pix)(COOL-2) | |
| FLJ26440 | 6 | NP_981932.1 | chromosome | Q6B4Z3 | Ubiquitously transcribed Y | Nuclear protein; Repeat; TPR repeat. |
| | | | 6 open reading | | chromosome tetratricopeptide | |
| | | | frame 71 | | repeat protein (Ubiquitously | |
| | | | [Homo sapiens] | | transcribed TPR protein on the Y | |
| | | | | | chromosome) | |
| FLJ21647 | 7 | NP_015561.1 | RAN binding | Q9H6Z4 | Ran-binding protein 3 (RanBP3) | Alternative splicing; Nuclear protein; Protein |
| | | | protein 3 isoform | | | transport; Transport. |
| | | | RANBP3-d | | | |
| | | | [Homo sapiens] | | | |
| FLJ26620 | 8 | NP_001738.2 | gelsolin-like | P40121 | Macrophage capping protein | 3D-structure; Actin capping; Actin-binding; |
| | | | capping protein | | (Actin-regulatoryprotein | Direct protein sequencing; Nuclear protein; |
| | | | [Homo sapiens] | | CAP-G) | Repeat. |

TABLE 7-2

| | | | | | | |
|---|---|---|---|---|---|---|
| FLJ43792 | 9 | NP_000400.2 | guanylate cyclase | P43080 | Guanylyl cyclase-activating protein | Calcium; Disease mutation; Lipoprotein; |
| | | | activator 1A (retina) | | 1 (GCAP 1) (Guanylate cyclase | Myristate; Repeat; Sensory transduction; |
| | | | [Homo sapiens] | | activator 1A) | Vision. |
| FLJ38127 | 10 | | | | | |
| FLJ35050 | 11 | NP_872271.1 | pyruvate kinase 3 | P11979 | Pyruvate kinase, isozyme M1 (EC | 3D-structure; Acetylation; Alternative splicing; |
| | | | isoform 2 | | 2.7.1.40) (Pyruvate kinase muscle | Direct protein sequencing; Glycolysis; Kinase; |
| | | | [Homo sapiens] | | isozyme) | Magnesium; Metal-binding; Multigene family; |
| | | | | | | Transferase. |
| FLJ27298 | 12 | NP_001655.1 | ras homolog gene | P61586 | Transforming protein RhoA (H12) | 3D-structure; ADP-ribosylation; Cytoskeleton; |
| | | | family, member A | | | Direct protein sequencing; GTP-binding; |
| | | | [Homo sapiens] | | | Lipoprotein; Magnesium; Membrane; |
| | | | | | | Methylation; Nucleotide-binding; Prenylation; |
| | | | | | | Proto-oncogene. |
| FLJ26262 | 13 | NP_001279.2 | chloride | O00299 | Chloride intracellular channel | 3D-structure; Acetylation; Chloride; Chloride |
| | | | intracellular | | protein 1 (Nuclear chloride | channel; Direct protein sequencing; Ion |
| | | | channel 1 | | ion channel 27) (NCC27) | transport; Ionic channel; Nuclear protein; |
| | | | [Homo sapiens] | | (p64 CLCP) (Chloride channel | Transport; Voltage-gated channel. |
| | | | | | ABP) (Regulatory nuclear | |
| | | | | | chloride ionchannel protein) | |
| | | | | | (hRNCC) | |
| FLJ90682 | 14 | NP_058625.1 | chloride | Q9EPT8 | Chloride intracellular channel | Chloride; Chloride channel; Ion transport; Ionic |
| | | | intracellular | | protein 5 | channel; Transport; Voltage-gated channel. |
| | | | channel 5 | | | |
| | | | [Homo sapiens] | | | |
| FLJ22923 | 15 | NP_005479.1 | target of myb1 | O60784 | Target of Myb protein 1 | 3D-structure; Membrane; Protein transport; |
| | | | [Homo sapiens] | | | Transport. |
| FLJ22871 | 16 | NP_612211.1 | polymerase | Q9Y535 | DNA-dependent RNA polymerase | Alternative splicing; DNA-dependent RNA |
| | | | (RNA) III (DNA | | III subunit 22.9 kDa polypeptide | polymerase; Nuclear protein; |
| | | | dependent) | | (EC 2.7.7.6) (RPC8) | Nucleotidyltransferase; Transcription; |
| | | | polypeptide H | | | Transferase. |
| | | | (22.9 kD) isoform a | | | |
| | | | [Homo sapiens] | | | |

TABLE 7-3

| | | | | | | |
|---|---|---|---|---|---|---|
| FLJ20398 | 17 | NP_055050.1 | ubiquitin-like 4 | P11441 | Ubiquitin-like protein 4 (Ubiquitin-likeprotein GDX) | |
| | | | [Homo sapiens] | | | |
| FLJ35377 | 18 | NP_613055.1 | ubiquitin-binding | | | |
| | | | protein homolog | | | |
| | | | [Mus musculus] | | | |
| FLJ42145 | 19 | NP_613055.1 | ubiquitin-binding | | | |
| | | | protein homolog | | | |
| | | | [Mus musculus] | | | |
| FLJ26144 | 20 | NP_612208.1 | glucosamine-6- | Q64422 | Glucosamine-6-phosphate isomerase (EC3.5.99.6) | Carbohydrate metabolism; |
| | | | phosphate | | (Glucosamine-6-phosphate deaminase) | Hydrolase. |
| | | | deaminase | | (GNPDA)(GlcN6P deaminase) (Oscillin) | |
| | | | 2 [Homo sapiens] | | | |
| FLJ26374 | 21 | NP_000166.2 | glucose phosphate | P06744 | Glucose-6-phosphate isomerase (EC 5.3.1.9) | 3D-structure; Acetylation; |
| | | | isomerase | | (GPI)(Phosphoglucose isomerase) (PGI) | Cytokine; Direct protein |
| | | | [Homo sapiens] | | (Phosphohexoseisomerase) (PHI) (Neuroleukin) | sequencing; Disease mutation; |
| | | | | | (NLK) (Sperm antigen 36)(SA-36) | Gluconeogenesis; Glycolysis; |
| | | | | | | Growth factor; Isomerase; |
| | | | | | | Polymorphism. |
| FLJ26371 | 22 | NP_002291.1 | lactate | P07195 | L-lactate dehydrogenase B chain (EC 1.1.1.27) | 3D-structure; Acetylation; |
| | | | dehydrogenase B | | (LDH-B) (LDH heart subunit) (LDH-H) | Direct protein sequencing; |
| | | | [Homo sapiens] | | | Disease mutation; Glycolysis; |
| | | | | | | Multigene family; NAD; |
| | | | | | | Oxidoreductase. |
| FLJ45688 | 23 | NP_817092.1 | protein | O15355 | Protein phosphatase 2C gamma isoform | Hydrolase; Magnesium; |
| | | | phosphatase | | (EC3.1.3.16) (PP2C-gamma) (Protein | Manganese; Metal-binding; |
| | | | 1G [Homo sapiens] | | phosphatasemagnesium-dependent 1 | Multigene family; Protein |
| | | | | | gamma) (Protein phosphatase 1C) | phosphatase. |

TABLE 7-4

| | | | | | | |
|---|---|---|---|---|---|---|
| FLJ38620 | 24 | NP_659190.2 | proline arginine rich | | | |
| | | | coiled coil 1 | | | |
| | | | [Mus musculus] | | | |
| FLJ26267 | 25 | NP_005380.1 | protein-L-isoaspartate (D- | P22061 | Protein-L-isoaspartate(D-aspartate)O- | 3D-structure; Acetylation; |
| | | | aspartate) O- | | methyltransferase (EC 2.1.1.77)(Protein- | Alternative splicing; Direct |
| | | | methyltransferase | | beta-aspartate methyltransferase) (PIMT) | protein sequencing; |
| | | | [Homo sapiens] | | (Protein L-isoaspartyl/D-aspartyl | Methyltransferase; |
| | | | | | methyltransferase)(L-isoaspartyl | Polymorphism; Transferase. |
| | | | | | protein carboxyl | |
| | | | | | methyltransferase) | |
| FLJ26062 | 26 | NP_006699.1 | glyoxalase I | Q04760 | Lactoylglutathione lyase | 3D-structure; Lyase; Metal- |
| | | | [Homo sapiens] | | (EC 4.4.1.5)(Methylglyoxalase) | binding; Polymorphism; Zinc. |
| | | | | | (Aldoketomutase) (Glyoxalase I) | |
| | | | | | (GlxI) (Ketone-aldehyde mutase) | |
| | | | | | (S-D-lactoylglutathionemethylglyoxal | |
| | | | | | lyase) | |
| FLJ22936 | 27 | NP_665799.1 | septin 6 isoform A | Q14141 | Septin-6 | Acetylation; Alternative splicing; |
| | | | [Homo sapiens] | | | Cell cycle; Cell division; Coiled |
| | | | | | | coil; Direct protein sequencing; |
| | | | | | | GTP-binding; Nucleotide- |
| | | | | | | binding. |
| FLJ43223 | 28 | NP_003671.1 | tyrosyl-tRNA synthetase | P54577 | Tyrosyl-tRNA synthetase, | 3D-structure; Acetylation; |
| | | | [Homo sapiens] | | cytoplasmic (EC6.1.1.1) | Aminoacyl-tRNA synthetase; |
| | | | | | (Tyrosyl-tRNA ligase) | ATP-binding; Direct protein |
| | | | | | (TyrRS) | sequencing; Ligase; Nucleotide- |
| | | | | | | binding; Protein biosynthesis; |
| | | | | | | RNA-binding; tRNA-binding. |

TABLE 7-5

| | | | | | | |
|---|---|---|---|---|---|---|
| FLJ26102 | 29 | NP_001850.1 | solute carrier family 31 (copper | O15431 | High-affinity copper uptake protein 1 | Copper; Copper transport; |
| | | | transporters), member 1 [Homo | | (hCTR1)(Copper transporter 1 ) (Solute | Ion transport; |
| | | | sapiens] | | carrier family 31 member1) | Transmembrane; Transport. |
| FLJ25218 | 30 | NP_872601.1 | tetratricopeptide repeat protein | Q6B4Z3 | Ubiquitously transcribed Y chromosome | Nuclear protein; Repeat; |
| | | | isoform 1 [Homo sapiens] | | tetratricopeptide repeat protein | TPR repeat. |
| | | | | | (Ubiquitously transcribed TPR protein | |
| | | | | | on the Y chromosome) | |
| FLJ45675 | 31 | | | Q8IVV7 | Protein C17 orf39 | |
| FLJ25918 | 32 | | | | | |
| FLJ46709 | 33 | NP_082185.1 | transmembrane protein 24 [Mus | Q80X80 | Transmembrane protein 24 | Transmembrane. |
| | | | musculus] | | | |
| FLJ40377 | 35 | | | | | |
| FLJ25845 | 36 | NP_775104.1 | armadillo repeat containing 3 | Q05609 | Serine/threonine-protein kinase CTR1 | ATP-binding; Ethylene |
| | | | [Homo sapiens] | | (EC2.7.1.37) | signaling pathway; Kinase; |
| | | | | | | Nucleotide-binding; Serine/ |
| | | | | | | threonine-protein kinase; |
| | | | | | | Transferase. |
| FLJ23662 | 37 | NP_060053.2 | DIPB protein [Homo sapiens] | Q96DX7 | Tripartite motif protein 44 (DIPB | Coiled coil; Metal-binding; |
| | | | | | protein) | Zinc; Zinc-finger. |
| FLJ12668 | 38 | NP_079273.2 | activating transcription factor 7 | Q6B4Z3 | Ubiquitously transcribed Y chromosome | Nuclear protein; Repeat; |
| | | | interacting protein 2 [Homo | | tetratricopeptide repeat protein | TPR repeat. |
| | | | sapiens] | | (Ubiquitously transcribed TPR protein | |
| | | | | | on the Y chromosome) | |
| FLJ90085 | 39 | NP_005484.2 | Ran binding protein 9 [Homo | Q96S59 | Ran-binding protein 9 (RanBP9) | Alternative splicing; |
| | | | sapiens] | | (RanBP7)(Ran-binding protein M) | Nuclear protein; |
| | | | | | (RanBPM) (BPM90) (BPM-L) | Phosphorylation; Ubl |
| | | | | | | conjugation. |

TABLE 7-6

| | | | | | | |
|---|---|---|---|---|---|---|
| FLJ90364 | 40 | NP_932156.1 | nudix -type motif 9 isoform | Q9BW91 | ADP-ribose pyrophosphatase, | 3D-structure; Alternative |
| | | | a [Homo sapiens] | | mitochondrialprecursor (EC 3.6.1.13) | splicing; Hydrolase; |
| | | | | | (ADP-ribose diphosphatase)(Adenosine | Magnesium; Manganese; |
| | | | | | diphosphoribose pyrophosphatase) | Mitochondrion; Transit |
| | | | | | (ADPR-PPase)(ADP-ribose | peptide. |
| | | | | | phosphohydrolase) (Nucleoside | |
| | | | | | diphosphate-linked moiety X | |
| | | | | | motif9) (Nudix motif9) | |
| FLJ90401 | 41 | NP_076995.1 | ELOVL family member 6, | Q9HB03 | Elongation of very long chain fatty | Endoplasmic reticulum; |
| | | | elongation of long chain | | acidsprotein 3 (Cold inducible | Fatty acid biosynthesis; |
| | | | fatty acids (FEN1/Elo2, | | glycoprotein of 30 kDa) | Lipid synthesis; |
| | | | SUR4/Elo3-like, yeast) | | | Transmembrane. |
| | | | [Homo sapiens] | | | |
| FLJ25526 | 42 | NP_008961.1 | brain-specific protein p25 | 094811 | Tubulin polymerization-promoting | Phosphorylation. |
| | | | alpha [Homo sapiens] | | protein(TPPP) (25 kDa brain-specific | |
| | | | | | protein) (p25-alpha) (p24)(p25) | |
| FLJ46896 | 43 | NP_032044.1 | SH3 multiple domains 1 [Mus | P14598 | Neutrophil cytosol factor 1 (NCF- | 3D-structure; Chronic |
| | | | musculus] | | 1)(Neutrophil NADPH oxidase factor 1) | granulomatous disease; |
| | | | | | (47 kDa neutrophiloxidase factor) | Disease mutation; |
| | | | | | (p47-phox) (NCF-47K) (47 kDa | Polymorphism; Repeat; SH3 |
| | | | | | autosomal chronic granulomatous | domain. |
| | | | | | disease protein) (NOXO2) | |
| FLJ46856 | 44 | NP_031489.2 | aortic preferentially | Q15772 | Aortic preferentially expressed | Immunoglobulin domain; |
| | | | expressed gene 1 [Mus | | protein 1(APEG-1) | Nuclear protein. |
| | | | musculus] | | | |
| FLJ90345 | 45 | NP_787071.2 | sine oculis homeobox homolog | Q8N196 | Homeobox protein SIX5 (DM locus- | Activator; Alternative |
| | | | 5 [Homo sapiens] | | associated homeodomain protein) | splicing; Developmental |
| | | | | | | protein; DNA-binding; |
| | | | | | | Homeobox; Nuclear protein; |
| | | | | | | Transcription; Transcription |
| | | | | | | regulation. |
| FLJ26550 | 46 | NP_006746.1 | transaldolase 1 [Homo | P37837 | Transaldolase (EC 2.2.1.2) | 3D-structure; Disease |
| | | | sapiens] | | | mutation; Pentose shunt; |
| | | | | | | Transferase. |

TABLE 7-7

| | | | | | | |
|---|---|---|---|---|---|---|
| FLJ90015 | 47 | NP_150638.1 | Mof4 family associated | | | |
| | | | protein 1 [Homo sapiens] | | | |
| FLJ39454 | 48 | NP_073745.2 | von Willebrand factor A | P32018 | Collagen alpha 1(XIV) chain | 3D-structure; Cell adhesion; Collagen; |
| | | | domain-associated protein | | precursor (Undulin) | Extracellular matrix; Glycoprotein; |
| | | | isoform 1 [Homo sapiens] | | | Hydroxylation; Repeat; Signal; |
| | | | | | | Structural protein. |
| FLJ45115 | 49 | NP_056224.2 | E1A binding protein p400 | Q96L91 | E1A binding protein p400 | Alternative splicing; ATP-bindng; |
| | | | [Homo sapiens] | | (EC 3.6.1.—) (p400 | Chromatin regulator; DNA-binding; |
| | | | | | kDaSWI2/SNF2- associated | Helicase; Hydrolase; Nuclear protein; |
| | | | | | protein) (Domino homolog) | Nucleotide-binding; Phosphorylation. |
| | | | | | (hDomino)(CAG repeat protein | |
| | | | | | 32) (Trinucleotide repeat- | |
| | | | | | containinggene 12 protein) | |
| FLJ90066 | 50 | NP_057648.2 | BM88 antigen [Homo | Q8N111 | BM88 antigen | Antigen; Transmembrane. |
| | | | sapiens] | | | |
| FLJ37995 | 51 | NP_940986.1 | carbonic anhydrase XIII | Q8N1Q1 | Carbonic anhydrase 13 (EC | Lyase; Metal-binding; Zinc. |
| | | | [Homo sapiens] | | 4.2.1.1) (Carbonicanhydrase | |
| | | | | | XIII) (Carbonate | |
| | | | | | dehydratase XIII) (CA-XIII) | |
| FLJ26058 | 52 | NP_001395.1 | eukaryotic translation | P26641 | Elongation factor 1-gamma | 3D-structure; Acetylation; Direct |
| | | | elongation factor 1 | | (EF-1-gamma) (eEF-1Bgamma) | protein sequencing; Elongation factor; |
| | | | gamma [Homo sapiens] | | | Protein biosynthesis. |
| FLJ46369 | 53 | | | | | |
| FLJ16517 | 54 | NP_665832.1 | RNA-binding protein | P21574 | Y-box binding protein 2-A | Direct protein sequencing; DNA- |
| | | | LIN-28 [Mus musculus] | | (CytoplasmicRNA-binding | binding; Nuclear protein; |
| | | | | | protein p56) (mRNP4) | Phosphorylation; RNA-binding; |
| | | | | | | Transcription; Transcription regulation. |
| FLJ26591 | 55 | NP_066953.1 | peptidylprolyl isomerase | P62941 | Peptidyl-prolyl cis-trans | Cyclosporin; Isomerase; Multigene |
| | | | A isoform 1 [Homo | | isomerase A (EC5.2.1.8) | family; Rotamase. |
| | | | sapiens] | | (PPIase) (Rotamase) | |
| | | | | | (Cyclophilin A)(Cyclosporin | |
| | | | | | A-binding protein) | |

TABLE 7-8

| | | | | | | |
|---|---|---|---|---|---|---|
| FLJ26596 | 56 | NP_066408.1 | H2A histone family, | P02261 | Histone H2A.c/d/i/n/p (H2A.1) | Acetylation; Chromosomal protein; |
| | | | member P [Homo | | (H2A/c) (H2A/d)(H2A/i) (H2A/n) | Direct protein sequencing; DNA-binding; |
| | | | sapiens] | | (H2A/p) (H2A.1b) | Multigene family; Nuclear protein; |
| | | | | | | Nucleosome core; Ubl conjugation. |
| FLJ90480 | 57 | NP_852149.1 | zinc finger, CCCH-type | Q8N5A5 | Zinc finger CCCH-type with G | Alternative splicing Metal-binding; |
| | | | with G patch domain | | patch domainprotein (Zinc | Zinc; Zinc-finger. |
| | | | isoform b [Homo | | finger CCCH- type domain | |
| | | | sapiens] | | containing protein9) | |
| FLJ43067 | 58 | NP_002620.1 | phosphoglycerate | P18669 | Phosphoglycerate mutase 1 (EC | 3D-structure; Acetylation; Direct |
| | | | mutase 1 (brain) | | 5.4.2.1) (EC5.4.2.4) (EC | protein sequencing; Glycolysis; |
| | | | [Homo sapiens] | | 3.1.3.13) (Phosphoglycerate | Hydrolase; Isomerase. |
| | | | | | mutase isozymeB) (PGAM-B) | |
| | | | | | (BPG-dependent PGAM 1) | |
| FLJ25460 | 59 | NP_620168.1 | ATPase, Class I, | 060423 | Probable phospholipid- | Alternative splicing ATP-bindng; |
| | | | type 8B, member 3 | | transporting ATPase IK(EC | Hydrolase; Magnesium; Metal-binding; |
| | | | [Homo sapiens] | | 3.6.3.1) (ATPase class I type | Multigene family; Nucleotide-binding; |
| | | | | | 8B member 3) | Phosphorylation; Transmembrane. |
| FLJ26806 | 60 | | | | | |
| FLJ43911 | 61 | | | | | |
| FLJ44715 | 62 | NP_775811.1 | fucosyltransferase 11 | P53992 | Protein transport protein | Endoplasmic reticulum; ER-Golgi |
| | | | (alpha (1, 3) | | Sec24C (SEC24-associated | transport; Golgi stack; Multigene |
| | | | fucosyltransferase) | | protein C) | family; Phosphorylation; Protein |
| | | | [Homo sapiens] | | | transport; Transport. |
| FLJ90031 | 63 | NP_036364.2 | polymerase I and | Q6NZI2 | Polymerase I and transcript | Acetylation; Alternative splicing; Direct |
| | | | transcript release | | release factor(PTRF protein) | protein sequencing; Membrane; |
| | | | factor [Homo sapiens] | | | Nuclear protein; Phosphorylation; |
| | | | | | | RNA-binding; rRNA-binding; |
| | | | | | | Transcription; Transcription regulation; |
| | | | | | | Transcription termination. |

Other examples of possible diseases or conditions are the diseases or conditions registered with OMIM. These diseases or conditions can easily be searched by, for example, inputting H-Inv ID numbers or H-Inv cluster ID numbers in H-Inv DB. The chromosomes and gene loci where the target genes for bioactive substances in this application are present, and OMIM information on orphan diseases expected to be associated with these genes, are shown in Tables 8-1 to 8-11.

TABLE 8-1
chromosome locus and OMIM disease information

| | | Chromosome | | | | |
|---|---|---|---|---|---|---|
| FLJ | Sequence | band | Genome locus | | | OMIM disease information |
| No. | No. | location | CLUSTER_START | CLUSTER_END | Strand | (OMIM Co-localized orphan disease) |
| FLJ21182 | 1 | 19p13.3 | 977298 | 997150 | + | OMIM_181800: SCOLIOSIS, IDIOPATHIC; |
| | | | | | | IS1, OMIM_602477: FEBRILE CONVULSIONS, |
| | | | | | | FAMILIAL, 2; FEB2, OMIM_145981: |
| | | | | | | HYPOCALCIURIC HYPERCALCEMIA, FAMILIAL, |
| | | | | | | TYPE II; HHC2, OMIM_601846: VACUOLAR |
| | | | | | | NEUROMYOPATHY, OMIM_609306: |
| | | | | | | SPINOCEREBELLAR ATAXIA 26; SCA26, |
| | | | | | | OMIM_108725: ATHEROSCLEROSIS |
| | | | | | | SUSCEPTIBILITY; ATHS, OMIM_606674: |
| | | | | | | INFLAMMATORY BOWEL DISEASE 6; IBD6, |
| | | | | | | OMIM_607508: MIGRAINE WITH OR WITHOUT |
| | | | | | | AURA, SUSCEPTIBILITY TO, 5, |
| | | | | | | OMIM_605364: PSORIASIS SUSCEPTIBILITY |
| | | | | | | 6, OMIM_125630: DERMODISTORTIVE |
| | | | | | | URTICARIA; DDU, OMIM_600209: EXOSTOSES, |
| | | | | | | MULTIPLE, TYPE III; EXT3, OMIM_120050: |
| | | | | | | COXSACKIEVIRUS B3 SUSCEPTIBILITY; CXB3S |
| FLJ38597 | 2 | 22q12.2 | 29801858 | 29825297 | + | OMIM_606960: INSULINOMA TUMOR |
| | | | | | | SUPPRESSOR GENE LOCUS, OMIM_608207: |
| | | | | | | KALA-AZAR, SUSCEPTIBILITY TO; KAZA, |
| | | | | | | OMIM_608908: MYOPIA 6, OMIM_604364: |
| | | | | | | EPILEPSY, PARTIAL, WITH VARIABLE FOCI, |
| | | | | | | OMIM_603116: CDAGS SYNDROME |
| FLJ13700 | 3 | 2p16.2 | 54596049 | 54808462 | + | OMIM_605244: CARNEY COMPLEX, TYPE II; |
| | | | | | | CNC2, OMIM_604254: DYSLEXIA, |
| | | | | | | SUSCEPTIBILITY TO, 3; DYX3, |
| | | | | | | OMIM_608703: SPINOCEREBELLAR ATAXIA |
| | | | | | | 25; SCA25, OMIM_137030: GALACTOSE + |
| | | | | | | ACTIVATOR; GLAT, OMIM_606415: |
| | | | | | | CANDIDIASIS, FAMILIAL CHRONIC |
| | | | | | | MUCOCUTANEOUS, AUTOSOMAL DOMINANT, |
| | | | | | | WITH THYROID DISEASE, OMIM_600666: |
| | | | | | | POLYCYSTIC KIDNEY DISEASE 3, |
| | | | | | | AUTOSOMAL DOMINANT; PKD3 |
| FLJ50683 | 4 | Xq23 | 114618464 | 114707970 | + | OMIM_300046: MENTAL RETARDATION, |
| | | | | | | X-LINKED 23; MRX23, OMIM_300046: |
| | | | | | | MENTAL RETARDATION, X-LINKED 23; |
| | | | | | | MRX23, OMIM_300324: MENTAL |
| | | | | | | RETARDATION, X-LINKED 53; MRX53, |
| | | | | | | OMIM_300464: CORONARY HEART DISEASE, |
| | | | | | | SUSCEPTIBILITY TO, 3, OMIM_301835: |
| | | | | | | ATAXIA, LETHAL X-LINKED, ACCOMPANYING |
| | | | | | | NANCHO AND BLINDNESS, OMIM_300158: |
| | | | | | | ARTHROGRYPOSIS, X-LINKED, TYPE V; |
| | | | | | | AMCX5, OMIM_300088: EPILEPSY, FEMALE- |
| | | | | | | RESTRICTED, WITH MENTAL RETARDATION; |
| | | | | | | EFMR, OMIM_300557: PARKINSON DISEASE |
| | | | | | | 12, OMIM_301201: AMELOGENESIS |
| | | | | | | IMPERFECTA, HYPOPLASTIC/HYPOMATURATION, |
| | | | | | | X-LINKED 2, OMIM_309300: MEGALOCORNEA; |
| | | | | | | MGC1, OMIM_300321: FG SYNDROME 2; FGS2, |
| | | | | | | OMIM_300125: MIGRAINE, FAMILIAL TYPICAL, |
| | | | | | | SUSCEPTIBILITY TO, 2, OMIM_300259: |
| | | | | | | MYCOBACTERIUM TUBERCULOSIS, SUSCEPTIBILITY |
| | | | | | | TO INFECTION, OMIM 300082: COGNITIVE |
| | | | | | | FUNCTION 1, SOCIAL; CGF1 |

TABLE 8-2

| | | | | | | |
|---|---|---|---|---|---|---|
| FLJ50199 | 5 | Xq26.3 | 135473228 | 135589767 | − | OMIM_309555: MENTAL RETARDATION WITH OPTIC ATROPHY, |
| | | | | | | DEAFNESS, AND SEIZURES, OMIM_313350: SPLIT-HAND/FOOT |
| | | | | | | MALFORMATION 2; SHFM2, OMIM_300700: ALBINISM-DEAFNESS |
| | | | | | | SYNDROME; ADFN, OMIM_307700: HYPOPARATHYROIDISM, X- |
| | | | | | | LINKED; HYPX, OMIM_300238: MENTAL RETARDATION, X- |
| | | | | | | LINKED, SYNDROMIC 11; MRXS11, OMIM_310700: NYSTAGMUS |
| | | | | | | 1, CONGENITAL, X-LINKED; NYS1, OMIM_300155: RETINITIS |
| | | | | | | PIGMENTOSA 24; RP24, OMIM_300179: X INACTIVATION, |
| | | | | | | FAMILIAL SKEWED, 2, OMIM_307150: HYPERTRICHOSIS, |
| | | | | | | CONGENITAL GENERALIZED; HTC2, OMIM_300245: PTOSIS, |
| | | | | | | HEREDITARY CONGENITAL 2, OMIM_300076: IMMUNONEUROLOGIC |
| | | | | | | DISORDER, X-LINKED, OMIM_313460: SURFACE ANTIGEN, |
| | | | | | | X-LINKED, SECONDARY; SAX2, OMIM_304340: DANDY-WALKER |
| | | | | | | MALFORMATION WITH MENTAL RETARDATION, BASAL GANGLIA |
| | | | | | | DISEASE, OMIM_300464: CORONARY HEART DISEASE, |
| | | | | | | SUSCEPTIBILITY TO, 3, OMIM_301845: BAZEX SYNDROME; BZX, |
| | | | | | | OMIM_300158: ARTHROGRYPOSIS, X-LINKED, TYPE V; AMCX5, |
| | | | | | | OMIM_304730: DERMOIDS OF CORNEA; CND, OMIM_301201: |
| | | | | | | AMELOGENESIS IMPERFECTA, HYPOPLASTIC/HYPOMATURATION, |
| | | | | | | X-LINKED 2, OMIM_309300: MEGALOCORNEA; MGC1, |
| | | | | | | OMIM_300321: FG SYNDROME 2; FGS2, OMIM_300125: MIGRAINE, |
| | | | | | | FAMILIAL TYPICAL, SUSCEPTIBILITY TO, 2, OMIM_300259: |
| | | | | | | MYCOBACTERIUM TUBERCULOSIS, SUSCEPTIBILITY TO INFECTION, |
| | | | | | | OMIM_300082: COGNITIVE FUNCTION 1, SOCIAL; CGF1 |
| FLJ26440 | 6 | 6q25.1 | 150782142 | 150817878 | + | OMIM_127500: DYSCHROMATOSIS UNIVERSALIS HEREDITARIA, |
| | | | | | | OMIM_180020: RETINAL CONE DYSTROPHY 1; RCD1, |
| | | | | | | OMIM_167000: TUMOR FORMATION SUPPRESSOR 8; ST8, |
| | | | | | | OMIM_606255: STATURE AS A QUANTITATIVE TRAIT, |
| | | | | | | OMIM_607446: BODY MASS INDEX QUANTITATIVE TRAIT LOCUS |
| | | | | | | ON CHROMOSOME NO 6, OMIM_608935: LUNG CANCER 1, |
| | | | | | | OMIM_603175: SCHIZOPHRENIA 5; SCZD5, OMIM_193007: |
| | | | | | | VESTIBULOPATHY, FAMILIAL |
| FLJ21647 | 7 | 19p13.3 | 5867154 | 5929165 | − | OMIM_181800: SCOLIOSIS, IDIOPATHIC; ISl, OMIM_602477: |
| | | | | | | FEBRILE CONVULSIONS, FAMILIAL, 2; FEB2, OMIM_145981: |
| | | | | | | HYPOCALCIURIC HYPERCALCEMIA, FAMILIAL, TYPE II; HHC2, |
| | | | | | | OMIM_601846: VACUOLAR NEUROMYOPATHY, OMIM_609306: |
| | | | | | | SPINOCEREBELLAR ATAXIA 26; SCA26, OMIM_108725: |
| | | | | | | ATHEROSCLEROSIS SUSCEPTIBILITY; ATHS, OMIM_606674: |
| | | | | | | INFLAMMATORY BOWEL DISEASE 6; IBD6, OMIM_607508: |
| | | | | | | MIGRAINE WITH OR WITHOUT AURA, SUSCEPTIBILITY TO, 5, |
| | | | | | | OMIM_605364: PSORIASIS SUSCEPTIBILITY 6, OMIM_125630: |
| | | | | | | DERMODISTORTIVE URTICARIA; DDU, OMIM_600209: EXOSTOSES, |
| | | | | | | MULTIPLE, TYPE III; EXT3, OMIM_120050: COXSACKIEVIRUS |
| | | | | | | B3 SUSCEPTIBILITY; CXB3S |
| FLJ26620 | 8 | 2p11.2 | 85533549 | 85552823 | − | OMIM_173340: PLASMINOGEN-LIKE; PLGL, OMIM_608394: |
| | | | | | | DEAFNESS, AUTOSOMAL DOMINANT NONSYNDROMIC SOUND |
| | | | | | | PERCEPTION) 43; DFNA43, OMIM_606068: RETINITIS |
| | | | | | | PIGMENTOSA 28; RP28, OMIM_137030: GALACTOSE + ACTIVATOR; |
| | | | | | | GLAT, OMIM_606415: CANDIDIASIS, FAMILIAL CHRONIC |
| | | | | | | MUCOCUTANEOUS, AUTOSOMAL DOMINANT, WITH THYROID DISEASE, |
| | | | | | | OMIM_600666: POLYCYSTIC KIDNEY DISEASE 3, AUTOSOMAL |
| | | | | | | DOMINANT; PKD3 |
| FLJ43792 | 9 | 6p21.1 | 42231152 | 42255770 | + | OMIM_609569: PHOTOPAROXYSMAL RESPONSE; PPR, OMIM_607498: |
| | | | | | | MIGRAINE WITH OR WITHOUT AURA, SUSCEPTIBILITY TO, 3, |
| | | | | | | OMIM_607017: DEAFNESS, AUTOSOMAL DOMINANT NONSYNDROMIC |
| | | | | | | SOUND PERCEPTION 21; DFNA21, OMIM_608645: DEAFNESS, |
| | | | | | | AUTOSOMAL DOMINANT NONSYNDROMIC SOUND PERCEPTION 31; |
| | | | | | | DFNA31, OMIM_608816: MYOCLONIC EPILEPSY, JUVENILE, 3, |
| | | | | | | OMIM_601086: LATERALITY DEFECTS, AUTOSOMAL DOMINANT, |
| | | | | | | OMIM_271250: SPINOCEREBELLAR ATAXIA, AUTOSOMAL RECESSIVE |
| | | | | | | 3; SCAR3, OMIM_122550: CORTICOSTERONE SIDE-CHAIN |
| | | | | | | ISOMERASE; CSCI, OMIM_604519: INFLAMMATORY BOWEL DISEASE |
| | | | | | | 3; IBD3, OMIM_143400: MULTICYSTIC RENAL DYSPLASIA, |
| | | | | | | BILATERAL; MRD |

TABLE 8-3

| | | | | | | |
|---|---|---|---|---|---|---|
| FLJ38127 | 10 | 5q33.2 | 153351456 | 153398663 | − | OMIM_605598: DIABETES MELLITUS, INSULIN-DEPENDENT, 18; |
| | | | | | | IDDM18, OMIM_608174: AUTOIMMUNE THYROID DISEASE, |
| | | | | | | SUSCEPTIBILITY TO, 2, OMIM_605845: DERMATITIS, ATOPIC, |
| | | | | | | 6; ATOD6, OMIM_131400: EOSINOPHILIA, FAMILIAL, |
| | | | | | | OMIM_602089: HEMANGIOMA, CAPILLARY INFANTILE, |
| | | | | | | OMIM_606348: INFLAMMATORY BOWEL DISEASE 5; IBD5, |
| | | | | | | OMIM_248310: PLASMODIUM FALCIPARUM BLOOD INFECTION |
| | | | | | | LEVEL, OMIM_181460: SCHISTOSOMA MANSONI INFECTION, |
| | | | | | | SUSCEPTIBILITY/RESISTANCE TO, OMIM_608970: MACULAR |
| | | | | | | DYSTROPHY, BUTTERFLY-SHAPED PIGMENTARY, 2, |
| | | | | | | OMIM_606070: MYOPATHY, DISTAL 2; MPD2 |
| FLJ35050 | 11 | 15q23 | 70256250 | 70310738 | − | OMIM_609439: DEAFNESS, AUTOSOMAL RECESSIVE 48; DFNB48, |
| | | | | | | OMIM_148600: KERATOSIS PALMOPLANTARIS PAPULOSA, |
| | | | | | | OMIM_607248: GLIOMA, FAMILIAL, 1, OMIM_105600: ANEMIA, |
| | | | | | | DYSERYTHROPOIETIC CONGENITAL, TYPE III; CDAN3, |
| | | | | | | OMIM_122460: CORONAVIRUS 229E SUSCEPTIBILITY; CVS, |
| | | | | | | OMIM_604329: HYPERTENSION, ESSENTIAL, SUSCEPTIBILITY |
| | | | | | | TO, 2, OMIM_214900: CHOLESTASIS-LYMPHEDEMA SYNDROME, |
| | | | | | | OMIM_214900: CHOLESTASIS-LYMPHEDEMA SYNDROME, |
| | | | | | | OMIM_609273: NEMALINE MYOPATHY 6; NEM6 |
| FLJ27298 | 12 | 3p21.31 | 49371582 | 49424530 | − | OMIM_225750: AICARDI-GOUTIERES SYNDROME 1; AGS1, |
| | | | | | | OMIM_192315: VASCULOPATHY, RETINAL, WITH CEREBRAL |
| | | | | | | LEUKODYSTROPHY, OMIM_606874: HIRSCHSPRUNG DISEASE, |
| | | | | | | SHORT-SEGMENT, 2, OMIM_605019: HYPOBETALIPOPROTEINEMIA, |
| | | | | | | FAMILIAL, 2, OMIM_182280: SMALL CELL CANCER OF THE LUNG, |
| | | | | | | OMIM_607135: CREATININE CLEARANCE QUANTITATIVE TRAIT |
| | | | | | | LOCUS, OMIM_601869: DEAFNESS, AUTOSOMAL RECESSIVE 15; |
| | | | | | | DFNB15, OMIM 142450: HERPESVIRUS SUSCEPTIBILITY; HV1S |
| FLJ26262 | 13 | 6p21.33 | 31806339 | 31813074 | − | OMIM_108800: ATRIAL SEPTAL DEFECT 1; ASD1, OMIM_606766: |
| | | | | | | AZOOSPERMIA, NONOBSTRUCTIVE, OMIM_137100: IMMUNOGLOBULIN |
| | | | | | | A DEFICIENCY 1; IGAD1, OMIM_146850: IMMUNE SUPPRESSION; |
| | | | | | | IS, OMIM_609148: MALARIA, MILD, SUSCEPTIBILITY TO, |
| | | | | | | OMIM_157860: MIXED LYMPHOCYTE CULTURE LOCUS II, |
| | | | | | | OMIM_607085: MYASTHENIA GRAVIS WITH THYMUS HYPERPLASIA, |
| | | | | | | OMIM_272370: SUSCEPTIBILITY TO LYSIS BY ALLOREACTIVE |
| | | | | | | NATURAL KILLER CELLS; EC1, OMIM_167250: PAGET DISEASE OF |
| | | | | | | BONE 1; PDB1, OMIM_176680: PRIMED LYMPHOCYTE TEST 1; PLT1, |
| | | | | | | OMIM_179450: RAGWEED SUSCEPTIBILITY, OMIM_608710: WEGENER |
| | | | | | | GRANULOMATOSIS, OMIM_603282: ZINC FINGER PROTEIN 204; |
| | | | | | | ZNF204, OMIM_150270: LARYNGEAL, ADDUCTOR PARALYSIS; LAP, |
| | | | | | | OMIM_607017: DEAFNESS, AUTOSOMAL DOMINANT NONSYNDROMIC |
| | | | | | | SOUND PERCEPTION 21; DFNA21, OMIM_608645: DEAFNESS, |
| | | | | | | AUTOSOMAL DOMINANT NONSYNDROMIC SOUND PERCEPTION 31; |
| | | | | | | DFNA31, OMIM_608816: MYOCLONIC EPILEPSY, JUVENILE, 3, |
| | | | | | | OMIM_601086: LATERALITY DEFECTS, AUTOSOMAL DOMINANT, |
| | | | | | | OMIM_608244: OTOSCLEROSIS 3; OTSC3, OMIM_271250: |
| | | | | | | SPINOCEREBELLAR ATAXIA, AUTOSOMAL RECESSIVE 3; SCAR3, |
| | | | | | | OMIM_122550: CORTICOSTERONE SIDE-CHAIN ISOMERASE; CSCI, |
| | | | | | | OMIM_604519: INFLAMMATORY BOWEL DISEASE 3; IBD3, |
| | | | | | | OMIM_143400: MULTICYSTIC RENAL DYSPLASIA, BILATERAL; MRD |

TABLE 8-4

| | | | | | | |
|---|---|---|---|---|---|---|
| FLJ90682 | 14 | 6p21.1 | 45977383 | 46156044 | − | OMIM_609569: PHOTOPAROXYSMAL RESPONSE; PPR, OMIM_607498: |
| | | | | | | MIGRAINE WITH OR WITHOUT AURA, SUSCEPTIBILITY TO, 3, |
| | | | | | | OMIM_607017: DEAFNESS, AUTOSOMAL DOMINANT NONSYNDROMIC |
| | | | | | | SOUND PERCEPTION 21; DFNA21, OMIM_608645: DEAFNESS, |
| | | | | | | AUTOSOMAL DOMINANT NONSYNDROMIC SOUND PERCEPTION 31; |
| | | | | | | DFNA31, OMIM_608816: MYOCLONIC EPILEPSY, JUVENILE, 3, |
| | | | | | | OMIM_601086: LATERALITY DEFECTS, AUTOSOMAL DOMINANT, |
| | | | | | | OMIM_271250: SPINOCEREBELLAR ATAXIA, AUTOSOMAL RECESSIVE |
| | | | | | | 3; SCAR3, OMIM_122550: CORTICOSTERONE SIDE-CHAIN |
| | | | | | | ISOMERASE; CSCI, OMIM_604519: INFLAMMATORY BOWEL DISEASE |
| | | | | | | 3; IBD3, OMIM_143400: MULTICYSTIC RENAL DYSPLASIA, |
| | | | | | | BILATERAL; MRD |
| FLJ22923 | 15 | 22q12.3 | 34020399 | 34068533 | + | OMIM_608207: KALA-AZAR, SUSCEPTIBILITY TO; KAZA, |
| | | | | | | OMIM_608908: MYOPIA 6, OMIM_604364: EPILEPSY, PARTIAL, |
| | | | | | | WITH VARIABLE FOCI, OMIM_603116: CDAGS SYNDROME |
| FLJ22871 | 16 | 22q13.2 | 40246308 | 40265110 | − | OMIM_603116: CDAGS SYNDROME |
| FLJ20398 | 17 | Xq28 | 153275762 | 153278675 | − | OMIM_300388: POLYMICROGYRIA, BILATERAL PERISYLVIAN, |
| | | | | | | OMIM_314400: CARDIAC VALVULAR DYSPLASIA, X-LINKED; |
| | | | | | | CVD1, OMIM_306995: HOMOSEXUALITY 1; HMS1, OMIM_300048: |
| | | | | | | INTESTINAL PSEUDOOBSTRUCTION, NEURONAL, CHRONIC |
| | | | | | | IDIOPATHIC, X-LINKED, OMIM_300271: MENTAL RETARDATION, |
| | | | | | | X-LINKED 72; MRX72, OMIM_300261: ARMFIELD X-LINKED |
| | | | | | | MENTAL RETARDATION SYNDROME, OMIM_300260: LUBS X-LINKED |
| | | | | | | MENTAL RETARDATION SYNDROME, OMIM_310460: MYOPIA 1; |
| | | | | | | MYP1, OMIM_314300: TORTICOLLIS, KELOIDS, CRYPTORCHIDISM, |
| | | | | | | AND RENAL DYSPLASIA; TKCR, OMIM_314900: XM SYSTEM, |
| | | | | | | OMIM_302000: BULLOUS DYSTROPHY, HEREDITARY MACULAR TYPE, |
| | | | | | | OMIM_300244: TERMINAL OSSEOUS DYSPLASIA AND PIGMENTARY |
| | | | | | | DEFECTS, OMIM_311510: PARKINSONISM, EARLY-ONSET, WITH |
| | | | | | | MENTAL RETARDATION, OMIM_301590: MICROPHTHALMIA, |
| | | | | | | SYNDROMIC 4; MCOPS4, OMIM_300147: PROSTATE CANCER, |
| | | | | | | HEREDITARY, X-LINKED; HPCX, OMIM_309200: MAJOR AFFECTIVE |
| | | | | | | DISORDER 2; MAFD2, OMIM_309620: MENTAL RETARDATION, |
| | | | | | | SKELETAL DYSPLASIA, AND ABDUCENS PALSY; MRSD, OMIM_300076: |
| | | | | | | IMMUNONEUROLOGIC DISORDER, X-LINKED, OMIM_313460: SURFACE |
| | | | | | | ANTIGEN, X-LINKED, SECONDARY; SAX2, OMIM_304730: DERMOIDS |
| | | | | | | OF CORNEA; CND, OMIM_304730: DERMOIDS OF CORNEA; CND, |
| | | | | | | OMIM_301201: AMELOGENESIS IMPERFECTA, HYPOPLASTIC/ |
| | | | | | | HYPOMATURATION, X-LINKED 2, OMIM_300321: FG SYNDROME 2; |
| | | | | | | FGS2, OMIM_300125: MIGRAINE, FAMILIAL TYPICAL, |
| | | | | | | SUSCEPTIBILITY TO, 2, OMIM_300259: MYCOBACTERIUM |
| | | | | | | TUBERCULOSIS, SUSCEPTIBILITY TO INFECTION, |
| | | | | | | OMIM_300082: COGNITIVE FUNCTION 1, SOCIAL; CGF1 |
| FLJ35377 | 18 | 16p12.1 | 23475893 | 23493216 | + | OMIM_608647: CILIARY DYSKINESIA, PRIMARY, 5, OMIM_602594: |
| | | | | | | RETINITIS PIGMENTOSA 22; RP22, OMIM_157700: MITRAL VALVE |
| | | | | | | PROLAPSE, FAMILIAL; MVP, OMIM_608105: EPILEPSY, ROLANDIC, |
| | | | | | | WITH PAROXYSMAL EXERCISE-INDUCED DYSTONIA AND, |
| | | | | | | OMIM_605013: MICROHYDRANENCEPHALY; MHAC, OMIM_606668: |
| | | | | | | INFLAMMATORY BOWEL DISEASE 8, OMIM_605751: CONVULSIONS, |
| | | | | | | BENIGN FAMILIAL INFANTILE, 2, OMIM_602066: CONVULSIONS, |
| | | | | | | FAMILIAL INFANTILE, WITH PAROXYSMAL CHOREOATHETOSIS |
| FLJ42145 | 19 | 16p12.1 | 23475893 | 23493216 | + | OMIM_608647: CILIARY DYSKINESIA, PRIMARY, 5, OMIM_602594: |
| | | | | | | RETINITIS PIGMENTOSA 22; RP22, OMIM_157700: MITRAL VALVE |
| | | | | | | PROLAPSE, FAMILIAL; MVP, OMIM_608105: EPILEPSY, ROLANDIC, |
| | | | | | | WITH PAROXYSMAL EXERCISE-INDUCED DYSTONIA AND, |
| | | | | | | OMIM_605013: MICROHYDRANENCEPHALY; MHAC, OMIM_606668: |
| | | | | | | INFLAMMATORY BOWEL DISEASE 8, OMIM_605751: CONVULSIONS, |
| | | | | | | BENIGN FAMILIAL INFANTILE, 2, OMIM_602066: CONVULSIONS, |
| | | | | | | FAMILIAL INFANTILE, WITH PAROXYSMAL CHOREOATHETOSIS |

TABLE 8-5

| | | | | | | |
|---|---|---|---|---|---|---|
| FLJ26144 | 20 | 4p13 | 44545085 | 44569540 | − | OMIM_106700: TOTAL ANOMALOUS PULMONARY VENOUS RETURN, |
| | | | | | | OMIM_607107: NASOPHARYNGEAL CARCINOMA 1, OMIM_605841: |
| | | | | | | NARCOLEPSY 2, OMIM_603663: MENTAL HEALTH WELLNESS 1 |
| FLJ26374 | 21 | 19q13.11 | 39547727 | 39584888 | + | OMIM_138972: CCAAT/ENHANCER-BINDING PROTEIN, GAMMA; |
| | | | | | | CEBPG, OMIM_604317: MICROCEPHALY, PRIMARY AUTOSOMAL |
| | | | | | | RECESSIVE, 2; MCPH2, OMIM_129150: ECHO VIRUS 11 |
| | | | | | | SUSCEPTIBILITY; E11S, OMIM_102699: ADENO-ASSOCIATED |
| | | | | | | VIRUS INTEGRATION SITE 1; AAVS1, OMIM_608542: ANEURYSM, |
| | | | | | | INTRACRANIAL BERRY, 2, OMIM_600740: HYPOCALCIURIC |
| | | | | | | HYPERCALCEMIA, FAMILIAL, TYPE III; HHC3, OMIM_609376: |
| | | | | | | CATARACT, CONGENITAL NUCLEAR, AUTOSOMAL RECESSIVE 1; |
| | | | | | | CATCN1, OMIM_600757: OROFACIAL CLEFT 3; OFC3, |
| | | | | | | OMIM_604805: SPASTIC PARAPLEGIA 12, AUTOSOMAL DOMINANT; |
| | | | | | | SPG12, OMIM_227240: EYE PIGMENTATION 1; EYCL1, |
| | | | | | | OMIM_113750: HAIR PIGMENTATION; HCL1, OMIM_601764: |
| | | | | | | CONVULSIONS, BENIGN FAMILIAL INFANTILE, 1, OMIM_606763: |
| | | | | | | CILIARY DYSKINESIA, PRIMARY, 2; CILD2, OMIM_607592: |
| | | | | | | PROSTATE CANCER AGGRESSIVENESS QUANTITATIVE TRAIT LOCUS |
| | | | | | | ON CHROMOSOME, OMIM_606712: SPECIFIC LANGUAGE IMPAIRMENT |
| | | | | | | 2; SLI2, OMIM_120050: COXSACKIEVIRUS B3 SUSCEPTIBILITY; |
| | | | | | | CXB3S |
| FLJ26371 | 22 | 12p12.1 | 21679542 | 21702043 | − | OMIM_603316: CYTIDINE 5-PRIME-MONOPHOSPHATE N- |
| | | | | | | ACETYLNEURAMINIC ACID SYNTHETASE, OMIM_608742: |
| | | | | | | HYPERTENSION, ESSENTIAL, SUSCEPTIBILITY TO, 4, |
| | | | | | | OMIM_208500: ASPHYXIATING THORACIC DYSTROPHY; ATD, |
| | | | | | | OMIM_208500: ASPHYXIATING THORACIC DYSTROPHY; ATD, |
| | | | | | | OMIM_112410: HYPERTENSION WITH BRACHYDACTYLY, |
| | | | | | | OMIM_107920: AROMATIC ALPHA-KETO ACID REDUCTASE, |
| | | | | | | OMIM_601458: INFLAMMATORY BOWEL DISEASE 2; IBD2, |
| | | | | | | OMIM_609113: TELOMERE LENGTH, MEAN LEUKOCYTE |
| FLJ45688 | 23 | 2p23.3 | 27515713 | 27544147 | − | OMIM_602134: TREMOR, HEREDITARY ESSENTIAL, 2; ETM2, |
| | | | | | | OMIM_606415: CANDIDIASIS, FAMILIAL CHRONIC |
| | | | | | | MUCOCUTANEOUS, AUTOSOMAL DOMINANT, WITH THYROID DISEASE, |
| | | | | | | OMIM_600666: POLYCYSTIC KIDNEY DISEASE 3, AUTOSOMAL |
| | | | | | | DOMINANT; PKD3 |
| FLJ38620 | 24 | 1p34.3 | 36290659 | 36315541 | + | OMIM_606713: VAN DER WOUDE SYNDROME 2, OMIM_609122: |
| | | | | | | ANEURYSM, INTRACRANIAL BERRY, 3, OMIM_608995: DYSLEXIA, |
| | | | | | | SUSCEPTIBILITY TO, 8; DYX8, OMIM_608446: MYOCARDIAL |
| | | | | | | INFARCTION, SUSCEPTIBILITY TO, 1, OMIM_121800: CORNEAL |
| | | | | | | DYSTROPHY, CRYSTALLINE, OF SCHNYDER, OMIM_606852: |
| | | | | | | PARKINSON DISEASE 10; PARK10, OMIM_605606: PSORIASIS |
| | | | | | | SUSCEPTIBILITY 7, OMIM_608543: SCHIZOPHRENIA 12 |
| FLJ26267 | 25 | 6q25.1 | 150162962 | 150224670 | + | OMIM_127500: DYSCHROMATOSIS UNIVERSALIS HEREDITARIA, |
| | | | | | | OMIM_180020: RETINAL CONE DYSTROPHY 1; RCD1, |
| | | | | | | OMIM_167000: TUMOR FORMATION SUPPRESSOR 8; ST8, |
| | | | | | | OMIM_606255: STATURE AS A QUANTITATIVE TRAIT, |
| | | | | | | OMIM_607446; BODY MASS INDEX QUANTITATIVE TRAIT |
| | | | | | | LOCUS ON CHROMOSOME NO 6, OMIM_608935: LUNG CANCER |
| | | | | | | 1, OMIM_603175: SCHIZOPHRENIA 5; SCZD5, OMIM_193007: |
| | | | | | | VESTIBULOPATHY, FAMILIAL |

TABLE 8-6

| | | | | | | |
|---|---|---|---|---|---|---|
| FLJ26062 | 26 | 6p21.2 | 38751698 | 38778895 | − | OMIM_150270: LARYNGEAL ADDUCTOR PARALYSIS; LAP, |
| | | | | | | OMIM_607017: DEAFNESS, AUTOSOMAL DOMINANT NONSYNDROMIC |
| | | | | | | SOUND PERCEPTION 21; DFNA21, OMIM_608645: DEAFNESS, |
| | | | | | | AUTOSOMAL DOMINANT NONSYNDROMIC SOUND PERCEPTION 31; |
| | | | | | | DFNA31, OMIM_608816: MYOCLONIC EPILEPSY, JUVENILE, |
| | | | | | | 3, OMIM_601086: LATERALITY DEFECTS, AUTOSOMAL DOMINANT, |
| | | | | | | OMIM_608244: OTOSCLEROSIS 3; OTSC3, OMIM_271250: |
| | | | | | | SPINOCEREBELLAR ATAXIA, AUTOSOMAL RECESSIVE 3; SCAR3, |
| | | | | | | OMIM_122550: CORTICOSTERONE SIDE-CHAIN ISOMERASE; CSCI, |
| | | | | | | OMIM_604519: INFLAMMATORY BOWEL DISEASE 3; IBD3, |
| | | | | | | OMIM_143400: MULTICYSTIC RENAL DYSPLASIA, BILATERAL; MRD |
| FLJ22936 | 27 | Xq24 | 118531572 | 118609215 | − | OMIM_300046: MENTAL RETARDATION, X-LINKED 23; MRX23, |
| | | | | | | OMIM_300046: MENTAL RETARDATION, X-LINKED 23; MRX23, |
| | | | | | | OMIM_300518: MENTAL RETARDATION, X-LINKED 82; MRX82, |
| | | | | | | OMIM_300354: MENTAL RETARDATION, X-LINKED, WITH SHORT |
| | | | | | | STATURE, SMALL TESTES, MUSCLE, OMIM_310490: COWCHOCK |
| | | | | | | SYNDROME, OMIM_300324: MENTAL RETARDATION, X-LINKED 53; |
| | | | | | | MRX53, OMIM_307150: HYPERTRICHOSIS, CONGENITAL |
| | | | | | | GENERALIZED; HTC2, OMIM_300245: PTOSIS, HEREDITARY |
| | | | | | | CONGENITAL 2, OMIM_300464: CORONARY HEART DISEASE, |
| | | | | | | SUSCEPTIBILITY TO, 3, OMIM_301845: BAZEX SYNDROME; BZX, |
| | | | | | | OMIM_301835: ATAXIA, LETHAL X-LINKED, ACCOMPANYING |
| | | | | | | NANCHO AND BLINDNESS, OMIM_300158: ARTHROGRYPOSIS, X- |
| | | | | | | LINKED, TYPE V; AMCX5, OMIM_304730: DERMOIDS OF CORNEA; |
| | | | | | | CND, OMIM_304730: DERMOIDS OF CORNEA; CND, OMIM_300088: |
| | | | | | | EPILEPSY, FEMALE-RESTRICTED, WITH MENTAL RETARDATION; |
| | | | | | | EFMR, OMIM_300557: PARKINSON DISEASE 12, OMIM_301201: |
| | | | | | | AMELOGENESIS IMPERFECTA, HYPOPLASTIC/HYPOMATURATION, |
| | | | | | | X-LINKED 2, OMIM_309300: MEGALOCORNEA; MGC1, OMIM_300321: |
| | | | | | | FG SYNDROME 2; FGS2, OMIM_300125: MIGRAINE, FAMILIAL |
| | | | | | | TYPICAL, SUSCEPTIBILITY TO, 2, OMIM_300259: MYCOBACTERIUM |
| | | | | | | TUBERCULOSIS, SUSCEPTIBILITY TO INFECTION, OMIM_300082: |
| | | | | | | COGNITIVE FUNCTION 1, SOCIAL; CGF1 |
| FLJ43223 | 28 | 1p35.1 | 32909933 | 32952847 | − | OMIM_132850: EPSTEIN-BARR VIRUS INSERTION SITE 1; EBVS1, |
| | | | | | | OMIM_609122: ANEURYSM, INTRACRANIAL BERRY, 3, OMIM_608995: |
| | | | | | | DYSLEXIA, SUSCEPTIBILITY TO, 8; DYX8, OMIM_608446: |
| | | | | | | MYOCARDIAL INFARCTION, SUSCEPTIBILITY TO, 1, OMIM_121800: |
| | | | | | | CORNEAL DYSTROPHY, CRYSTALLINE, OF SCHNYDER, OMIM_606852: |
| | | | | | | PARKINSON DISEASE 10; PARK10, OMIM_605606: PSORIASIS |
| | | | | | | SUSCEPTIBILITY 7, OMIM_608543: SCHIZOPHRENIA 12 |
| FLJ26102 | 29 | 9q32 | 113063362 | 113108769 | + | OMIM_154400: ACROFACIAL DYSOSTOSIS 1, NAGER TYPE; AFD1, |
| | | | | | | OMIM_608026: HYPERTENSIVE NEPHROPATHY, OMIM_608762: |
| | | | | | | EPILEPSY, IDIOPATHIC GENERALIZED, SUSCEPTIBILITY TO, 3; |
| | | | | | | EIG3, OMIM_607152: SPASTIC PARAPLEGIA 19, AUTOSOMAL |
| | | | | | | DOMINANT; SPG19 |
| FLJ25218 | 30 | 12q14.3 | 64803109 | 64810800 | − | OMIM_609195: SPASTIC PARAPLEGIA 26, AUTOSOMAL RECESSIVE; |
| | | | | | | SPG26, OMIM_606257: STATURE QUANTITATIVE TRAIT LOCUS 3, |
| | | | | | | OMIM_600808: ENURESIS, NOCTURNAL, 2; ENUR2, OMIM_102300: |
| | | | | | | RESTLESS LEGS SYNDROME, SUSCEPTIBILITY TO, 1, |
| | | | | | | OMIM_102300: RESTLESS LEGS SYNDROME, SUSCEPTIBILITY TO, |
| | | | | | | 1, OMIM_121400: CORNEA PLANA 1; CNA1, OMIM_601458: |
| | | | | | | INFLAMMATORY BOWEL DISEASE 2; IBD2, OMIM_609113: |
| | | | | | | TELOMERE LENGTH, MEAN LEUKOCYTE |
| FLJ45675 | 31 | 17p11.2 | 17883331 | 17912444 | + | OMIM_607354: SCOLIOSIS, IDIOPATHIC, SUSCEPTIBILITY TO, |
| | | | | | | 2; IS2, OMIM_604547: VAN DER WOUDE SYNDROME MODIFIER, |
| | | | | | | OMIM_608904: ATTENTION DEFICIT-HYPERACTIVITY DISORDER, |
| | | | | | | SUSCEPTIBILITY TO, 2, OMIM_215500: CHOROIDAL DYSTROPHY, |
| | | | | | | CENTRAL AREOLAR; CACD, OMIM_601251: RETINAL CONE |
| | | | | | | DYSTROPHY 2 |

TABLE 8-7

| | | | | | | |
|---|---|---|---|---|---|---|
| FLJ25918 | 32 | 16p13.3 | 4451693 | 4466308 | − | OMIM_156850: MICROPHTHALMIA, ISOLATED, WITH CATARACT 1; |
| | | | | | | MCOPCT1, OMIM_608903: ATTENTION DEFICIT-HYPERACTIVITY |
| | | | | | | DISORDER, SUSCEPTIBILITY TO, 1, OMIM_608558: BODY MASS |
| | | | | | | INDEX QUANTITATIVE TRAIT LOCUS ON CHROMOSOME NO 16, IN |
| | | | | | | CHILDREN, OMIM_607339: CORONARY HEART DISEASE, |
| | | | | | | SUSCEPTIBILITY TO, 1, OMIM_605021: MYOCLONIC EPILEPSY, |
| | | | | | | INFANTILE, OMIM_605013: MICROHYDRANENCEPHALY; MHAC, |
| | | | | | | OMIM_606668: INFLAMMATORY BOWEL DISEASE 8, |
| FLJ46709 | 33 | 21q22.3 | 42178290 | 42247068 | − | OMIM_236100: HOLOPROSENCEPHALY, OMIM_609428: TUKEL |
| | | | | | | SYNDROME |
| RGNpc017 | 34 | 14q32.11 | 89933126 | 89944362 | + | OMIM_608318: CORONARY HEART DISEASE, SUSCEPTIBILITY TO, |
| | | | | | | 4, OMIM_123270: CREATINE KINASE, BRAIN TYPE, ECTOPIC |
| | | | | | | EXPRESSION OF; CKBE, OMIM_164210: HEMIFACIAL MICROSOMIA; |
| | | | | | | HEM, OMIM_251600: MICROPHTHALMIA, ISOLATED 1; MCOP1, |
| | | | | | | OMIM_115650: CATARACT, ANTERIOR POLAR, 1; CTAA1, |
| | | | | | | OMIM_213600: BASAL GANGLIA CALCIFICATION, IDIOPATHIC, 1; |
| | | | | | | IBGC1, OMIM_138800: GOITER, MULTINODULAR 1; MNG1 |
| FLJ40377 | 35 | 19q13.33 | 54583318 | 54613062 | + | OMIM_605589: CHARCOT-MARIE-TOOTH DISEASE, AXONAL, TYPE |
| | | | | | | 2B2; CMT2B2, OMIM_271930: STRIATONIGRAL DEGENERATION, |
| | | | | | | INFANTILE; SNDI, OMIM_604559: PROGRESSIVE FAMILIAL |
| | | | | | | HEART BLOCK, TYPE I, LOCUS1, OMIM_129150: ECHO VIRUS 11 |
| | | | | | | SUSCEPTIBILITY; E11S, OMIM_603855: CYSTIC FIBROSIS |
| | | | | | | MODIFIER 1; CFM1, OMIM_102699: ADENO-ASSOCIATED VIRUS |
| | | | | | | INTEGRATION SITE 1; AAVS1, OMIM_608542: ANEURYSM, |
| | | | | | | INTRACRANIAL BERRY, 2, OMIM_600740: HYPOCALCIURIC |
| | | | | | | HYPERCALCEMIA, FAMILIAL, TYPE III; HHC3, OMIM_609376: |
| | | | | | | CATARACT, CONGENITAL NUCLEAR, AUTOSOMAL RECESSIVE 1; |
| | | | | | | CATCN1, OMIM_600757: OROFACIAL CLEFT 3; OFC3, |
| | | | | | | OMIM_604805: SPASTIC PARAPLEGIA 12, AUTOSOMAL DOMINANT; |
| | | | | | | SPG12, OMIM_601764: CONVULSIONS, BENIGN FAMILIAL |
| | | | | | | INFANTILE, 1, OMIM_606763: CILIARY DYSKINESIA, PRIMARY, |
| | | | | | | 2; CILD2, OMIM_607592: PROSTATE CANCER AGGRESSIVENESS |
| | | | | | | QUANTITATIVE TRAIT LOCUS ON CHROMOSOME, OMIM_606712: |
| | | | | | | SPECIFIC LANGUAGE IMPAIRMENT 2; SLI2, OMIM_120050: |
| | | | | | | COXSACKIEVIRUS B3 SUSCEPTIBILITY; CXB3S |
| FLJ25845 | 36 | 10p12.2 | 23256966 | 23366520 | + | OMIM_604401: ARRHYTHMOGENIC RIGHT VENTRICULAR DYSPLASIA, |
| | | | | | | FAMILIAL, 6, OMIM_600964: INCREASE REFSUM DISEASE WITH |
| | | | | | | PIPECOLIC ACIDEMIA; RDPA, OMIM_603188: OBESITY, |
| | | | | | | SUSCEPTIBILITY TO, ON CHROMOSOME 10p; OB10P |
| FLJ23662 | 37 | 11p13 | 35640929 | 35786333 | + | OMIM_609256: MYOPIA 7, OMIM_609941: DEAFNESS, AUTOSOMAL |
| | | | | | | RECESSIVE 51; DFNB51, OMIM_605750: EXUDATIVE |
| | | | | | | VITREORETINOPATHY 3; EVR3, OMIM_604499: HYPERLIPIDEMIA, |
| | | | | | | COMBINED, 2 |
| FLJ12668 | 38 | 16p13.13 | 10387413 | 10484995 | + | OMIM_608903: ATTENTION DEFICIT-HYPERACTIVITY DISORDER, |
| | | | | | | SUSCEPTIBILITY TO, 1, OMIM_608558: BODY MASS INDEX |
| | | | | | | QUANTITATIVE TRAIT LOCUS ON CHROMOSOME NO 16, IN |
| | | | | | | CHILDREN, OMIM_607339: CORONARY HEART DISEASE, |
| | | | | | | SUSCEPTIBILITY TO, 1, OMIM_605021: MYOCLONIC EPILEPSY, |
| | | | | | | INFANTILE, OMIM_605013: MICROHYDRANENCEPHALY; MHAC, |
| | | | | | | OMIM_606668: INFLAMMATORY BOWEL DISEASE 8, |

TABLE 8-8

| | | | | | | |
|---|---|---|---|---|---|---|
| FLJ90085 | 39 | 12q13.13 | 51744656 | 51759437 | − | OMIM_607936: EXFOLIATIVE ICHTHYOSIS, AUTOSOMAL RECESSIVE, |
| | | | | | | ICHTHYOSIS BULLOSA OF SIEMENS-LIKE, OMIM_167960: HUMAN |
| | | | | | | PAPILLOMAVIRUS TYPE 18 INTEGRATION SITE 2; HPV18I2, |
| | | | | | | OMIM_607598: LETHAL CONGENITAL CONTRACTURE SYNDROME 2, |
| | | | | | | OMIM_608591: CHARCOT-MARIE-TOOTH DISEASE, AXONAL, TYPE |
| | | | | | | 2G; CMT2G, OMIM 609195: SPASTIC PARAPLEGIA 26, AUTOSOMAL |
| | | | | | | RECESSIVE; SPG26, OMIM_606257: STATURE QUANTITATIVE TRAIT |
| | | | | | | LOCUS 3, OMIM_600808: ENURESIS, NOCTURNAL, 2; ENUR2, |
| | | | | | | OMIM_102300: RESTLESS LEGS SYNDROME, SUSCEPTIBILITY TO, 1, |
| | | | | | | OMIM_102300: RESTLESS LEGS SYNDROME, SUSCEPTIBILITY TO, 1, |
| | | | | | | OMIM_121400: CORNEA PLANA 1; CNA1, OMIM_601458: |
| | | | | | | INFLAMMATORY BOWEL DISEASE 2; IBD2, OMIM_609113: TELOMERE |
| | | | | | | LENGTH, MEAN LEUKOCYTE |
| FLJ90364 | 40 | 4q22.1 | 88700914 | 88737785 | + | OMIM_147060: HYPERIMMUNOGLOBULIN E RECURRENT INFECTION |
| | | | | | | SYNDROME, OMIM_609115: LIMB-GIRDLE MUSCULAR DYSTROPHY, |
| | | | | | | TYPE 1G; LGMD1G, OMIM_604928: WOLFRAM SYNDROME 2; WFS2, |
| | | | | | | OMIM_151001: LENTIGINOSIS, INHERITED PATTERNED, |
| | | | | | | OMIM_609566: PARIETAL FORAMINA 3; PFM3, OMIM_609400: |
| | | | | | | AUTOIMMUNE DISEASE, SUSCEPTIBILITY TO, 4, OMIM_134720: |
| | | | | | | FECUNDITY GENE, BOOROOLA, OF SHEEP, HOMOLOG OF, |
| | | | | | | OMIM_605841: NARCOLEPSY 2, OMIM_608371: OROFACIAL CLEFT 4, |
| | | | | | | OMIM_603664: MENTAL HEALTH WELLNESS 2, OMIM_601454: |
| | | | | | | PSORIASIS SUSCEPTIBILITY 3; PSORS3 |
| FLJ90401 | 41 | 4q25 | 111328127 | 111477375 | − | OMIM_138900: GOUT, SUSCEPTIBILITY TO 1, OMIM_606460: |
| | | | | | | LONGEVITY 1, OMIM_134720: FECUNDITY GENE, BOOROOLA, OF |
| | | | | | | SHEEP, HOMOLOG OF, OMIM_608371: OROFACIAL CLEFT 4, |
| | | | | | | OMIM_603664: MENTAL HEALTH WELLNESS 2, OMIM_601454: |
| | | | | | | PSORIASIS SUSCEPTIBILITY 3; PSORS3 |
| FLJ25526 | 42 | 5p15.33 | 712978 | 746466 | − | OMIM_601888: MALIGNANT HYPERTHERMIA, SUSCEPTIBILITY TO, 6 |
| FLJ46896 | 43 | 5q35.1 | 171684794 | 171814132 | − | OMIM_208100: ARTHROGRYPOSIS MULTIPLEX CONGENITA, |
| | | | | | | NEUROGENIC TYPE; AMCN, OMIM_118840: CHROMATE RESISTANCE; |
| | | | | | | CHR, OMIM_606070: MYOPATHY, DISTAL 2; MPD2 |
| FLJ46856 | 44 | 2q35 | 220127502 | 220183855 | + | OMIM_607949: MYCOBACTERIUM TUBERCULOSIS, SUSCEPTIBILITY |
| | | | | | | TO, 1, OMIM_607966: SYSTEMIC LUPUS ERYTHEMATOSUS WITH |
| | | | | | | NEPHRITIS, SUSCEPTIBILITY TO, 2;, OMIM_609153: |
| | | | | | | PSEUDOHYPERKALEMIA, FAMILIAL, 2, DUE TO RED CELL LEAK, |
| | | | | | | OMIM_262000: PILI TORTI AND NERVE DEAFNESS, OMIM_185900: |
| | | | | | | SYNDACTYLY, TYPE I, OMIM_185900: SYNDACTYLY, TYPE I, |
| | | | | | | OMIM_601286: CATARACT, NONNUCLEAR POLYMORPHIC CONGENITAL, |
| | | | | | | AUTOSOMAL DOMINANT, OMIM_606053: AUTISM, SUSCEPTIBILITY |
| | | | | | | TO, 5; AUTS5, OMIM_606963: PULMONARY DISEASE, CHRONIC |
| | | | | | | OBSTRUCTIVE, SEVERE EARLY-ONSET |

TABLE 8-9

| | | | | | | |
|---|---|---|---|---|---|---|
| FLJ90345 | 45 | 19q13.32 | 50959884 | 50964783 | − | OMIM_605589: CHARCOT-MARIE-TOOTH DISEASE, AXONAL, TYPE |
| | | | | | | 2B2; CMT2B2, OMIM_271930: STRIATONIGRAL DEGENERATION, |
| | | | | | | INFANTILE; SNDI, OMIM_604559: PROGRESSIVE FAMILIAL HEART |
| | | | | | | BLOCK, TYPE I, LOCUS1, OMIM_129150: ECHO VIRUS 11 |
| | | | | | | SENSITIVITY; E11S, OMIM_603855: CYSTIC FIBROSIS MODIFIER |
| | | | | | | 1; CFM1, OMIM_102699: ADENO-ASSOCIATED VIRUS INTEGRATION |
| | | | | | | SITE 1; AAVS1, OMIM_608542: ANEURYSM, INTRACRANIAL BERRY, |
| | | | | | | 2, OMIM_600740: HYPOCALCIURIC HYPERCALCEMIA, FAMILIAL, |
| | | | | | | TYPE III; HHC3, OMIM_609376: CATARACT, CONGENITAL NUCLEAR, |
| | | | | | | AUTOSOMAL RECESSIVE 1; CATCN1, OMIM_600757: OROFACIAL |
| | | | | | | CLEFT 3; OFC3, OMIM_604805: SPASTIC PARAPLEGIA 12, |
| | | | | | | AUTOSOMAL DOMINANT; SPG12, OMIM_601764: CONVULSIONS, |
| | | | | | | BENIGN FAMILIAL INFANTILE, 1, OMIM_606763: CILIARY |
| | | | | | | DYSKINESIA, PRIMARY, 2; CILD2, OMIM_607592: PROSTATE |
| | | | | | | CANCER AGGRESSIVENESS QUANTITATIVE TRAIT LOCUS ON |
| | | | | | | CHROMOSOME, OMIM_606712: SPECIFIC LANGUAGE IMPAIRMENT 2; |
| | | | | | | SLI2, OMIM_120050: COXSACKIEVIRUS B3 SUSCEPTIBILITY; CXB3S |
| FLJ26550 | 46 | 11p15.5 | 737427 | 755023 | + | OMIM_607967: SYSTEMIC LUPUS ERYTHEMATOSUS WITH NEPHRITIS, |
| | | | | | | SUSCEPTIBILITY TO, 3;, OMIM_194071: MULTIPLE TUMOR RELATED |
| | | | | | | CHROMOSOMAL REGION 1; MTACR1, OMIM_609470: NONCOMPACTION |
| | | | | | | OF LEFT VENTRICULAR MYOCARDIUM, FAMILIAL ISOLATED, |
| | | | | | | AUTOSOMAL DOMINANT, OMIM_609270: SPINOCEREBELLAR ATAXIA, |
| | | | | | | AUTOSOMAL RECESSIVE 7; SCAR7, OMIM_604499: HYPERLIPIDEMIA, |
| | | | | | | COMBINED, 2 |
| FLJ90015 | 47 | 4p16.1 | 6759890 | 6762544 | + | OMIM_603663: MENTAL HEALTH WELLNESS 1 |
| FLJ39454 | 48 | 1p36.33 | 1456176 | 1463524 | + | OMIM_606928: BONE MINERAL DENSITY VARIATION 3; BMND3, |
| | | | | | | OMIM_211420: BREAST CANCER, DUCTAL, 2; BRCD2, OMIM_115665: |
| | | | | | | CATARACT, CONGENITAL, VOLKMANN TYPE; CCV, OMIM_155600: |
| | | | | | | MELANOMA, CUTANEOUS MALIGNANT; CMM, OMIM_116600: CATARACT, |
| | | | | | | POSTERIOR POLAR, 1, OMIM_607671: DYSTONIA 13, TORSION; |
| | | | | | | DYT13, OMIM_600975: GLAUCOMA 3, PRIMARY INFANTILE, B; |
| | | | | | | GLC3B, OMIM_605225: INFLAMMATORY BOWEL DISEASE 7; IBD7, |
| | | | | | | OMIM_608553: LEBER CONGENITAL AMAUROSIS, TYPE IX, |
| | | | | | | OMIM_606693: KUFOR-RAKEB SYNDROME; KRS, OMIM_607317: |
| | | | | | | SPINOCEREBELLAR ATAXIA, AUTOSOMAL RECESSIVE 4; SCAR4, |
| | | | | | | OMIM_608995: DYSLEXIA, SUSCEPTIBILITY TO, 8; DYX8, |
| | | | | | | OMIM_608446: MYOCARDIAL INFARCTION, SUSCEPTIBILITY TO, 1, |
| | | | | | | OMIM_121800: CORNEAL DYSTROPHY, CRYSTALLINE, OF SCHNYDER, |
| | | | | | | OMIM_606852: PARKINSON DISEASE 10; PARK10, OMIM_605606: |
| | | | | | | PSORIASIS SUSCEPTIBILITY 7, OMIM_608543: SCHIZOPHRENIA 12 |
| FLJ45115 | 49 | 12q24.33 | 131100735 | 131231241 | + | OMIM_608447: CAROTID INTIMAL MEDIAL THICKNESS 2, |
| | | | | | | OMIM_608224: DEAFNESS, AUTOSOMAL DOMINANT NONSYNDROMIC |
| | | | | | | SOUND PERCEPTION 41; DFNA41, OMIM_608437: SYSTEMIC LUPUS |
| | | | | | | ERYTHEMATOSUS, SUSCEPTIBILITY TO, 4, OMIM_606071: |
| | | | | | | HEREDITARY MOTOR AND SENSORY NEUROPATHY, TYPE IIC, |
| | | | | | | OMIM_600175: SPINAL MUSCULAR ATROPHY, CONGENITAL |
| | | | | | | NONPROGRESSIVE, DISTAL, OMIM_605583: DEAFNESS, AUTOSOMAL |
| | | | | | | DOMINANT NONSYNDROMIC SOUND PERCEPTION 25; DFNA25, |
| | | | | | | OMIM_121400: CORNEA PLANA 1; CNA1, OMIM_609113: |
| | | | | | | TELOMERE LENGTH, MEAN LEUKOCYTE |
| FLJ90066 | 50 | 11p15.5 | 777104 | 780123 | − | OMIM_607967: SYSTEMIC LUPUS ERYTHEMATOSUS WITH NEPHRITIS, |
| | | | | | | SUSCEPTIBILITY TO, 3;, OMIM_194071: MULTIPLE TUMOR |
| | | | | | | RELATED CHROMOSOMAL REGION 1; MTACR1, OMIM_609470: |
| | | | | | | NONCOMPACTION OF LEFT VENTRICULAR MYOCARDIUM, FAMILIAL |
| | | | | | | ISOLATED, AUTOSOMAL DOMINANT, OMIM_609270: SPINOCEREBELLAR |
| | | | | | | ATAXIA, AUTOSOMAL RECESSIVE 7; SCAR7, OMIM_604499: |
| | | | | | | HYPERLIPIDEMIA, COMBINED, 2 |

TABLE 8-10

| | | | | | | |
|---|---|---|---|---|---|---|
| FLJ37995 | 51 | 8q21.2 | 86320097 | 86548526 | + | OMIM_187280: TEMPERATURE SUSCEPTIBILITY COMPLEMENTATION, |
| | | | | | | CELL CYCLE SPECIFIC, tsBN51, OMIM_121210: FEBRILE |
| | | | | | | CONVULSIONS, FAMILIAL, 1; FEB1, OMIM_121210: FEBRILE |
| | | | | | | CONVULSIONS, FAMILIAL, 1; FEB1, OMIM_600668: |
| | | | | | | CHONDROCALCINOSIS 1; CCAL1, OMIM_606789: FETAL HEMOGLOBIN |
| | | | | | | QUANTITATIVE TRAIT LOCUS ON CHROMOSOME NO 8 |
| FLJ26058 | 52 | 11q12.3 | 62083649 | 62115592 | − | OMIM_135610: FIBRONECTIN-LIKE 2; FNL2, OMIM_608091: |
| | | | | | | JOUBERT SYNDROME 2; JBTS2 |
| FLJ46369 | 53 | 17q12 | 31244824 | 31262140 | − | OMIM_601363: WILMS TUMOR 4, OMIM_161000: NAEGELI SYNDROME, |
| | | | | | | OMIM_603918: HYPERTENSION, ESSENTIAL, SUSCEPTIBILITY TO, |
| | | | | | | 1, OMIM_602723: PSORIASIS SUSCEPTIBILITY 2; PSORS2 |
| FLJ16517 | 54 | 6q21 | 105511616 | 105635514 | + | OMIM_606325: HETEROTAXY, VISCERAL, 3, OMIM_601666: |
| | | | | | | DIABETES MELLITUS, INSULIN-DEPENDENT, 15; IDDM15, |
| | | | | | | OMIM_218400: CRANIOMETAPHYSEAL DYSPLASIA, AUTOSOMAL |
| | | | | | | RECESSIVE; CMDR, OMIM_608852: PULMONARY FUNCTION, |
| | | | | | | OMIM_608988: ATRIAL FIBRILLATION, FAMILIAL, 3; ATFB3, |
| | | | | | | OMIM_602772: RETINITIS PIGMENTOSA 25; RP25, OMIM_605582: |
| | | | | | | CARDIOMYOPATHY, DILATED, 1K; CMD1K, OMIM_604537: LEBER |
| | | | | | | CONGENITAL AMAUROSIS, TYPE V, OMIM_603175: SCHIZOPHRENIA |
| | | | | | | 5; SCZD5, OMIM_193007: VESTIBULOPATHY, FAMILIAL |
| FLJ26591 | 55 | 7p13 | 44609492 | 44615955 | + | OMIM_141400: HEMIFACIAL MICROSOMIA WITH RADIAL DEFECTS |
| FLJ26596 | 56 | 6p22.1 | 27911265 | 27915239 | − | OMIM_600511: SCHIZOPHRENIA 3; SCZD3, OMIM_608244: |
| | | | | | | OTOSCLEROSIS 3; OTSC3, OMIM_271250: SPINOCEREBELLAR |
| | | | | | | ATAXIA, AUTOSOMAL RECESSIVE 3; SCAR3, OMIM_122550: |
| | | | | | | CORTICOSTERONE SIDE-CHAIN ISOMERASE; CSCI, OMIM_604519: |
| | | | | | | INFLAMMATORY BOWEL DISEASE 3; IBD3, OMIM_143400: |
| | | | | | | MULTICYSTIC RENAL DYSPLASIA, BILATERAL; MRD |
| FLJ90480 | 57 | 20q13.33 | 61809260 | 61840900 | + | OMIM_130180: ELECTROENCEPHALOGRAM, LOW-VOLTAGE, |
| | | | | | | OMIM_608656: PROSTATE CANCER, HEREDITARY, 3, OMIM_608029: |
| | | | | | | SPINOCEREBELLAR ATAXIA, AUTOSOMAL RECESSIVE 6; SCAR6 |
| FLJ43067 | 58 | 10q24.1 | 99175940 | 99183188 | + | OMIM_601162: SPASTIC PARAPLEGIA 9, AUTOSOMAL DOMINANT; |
| | | | | | | SPG9, OMIM_606483: CHARCOT-MARIE-TOOTH DISEASE, DOMINANT |
| | | | | | | INTERMEDIATE A, OMIM_602082: CORNEAL DYSTROPHY OF BOWMAN |
| | | | | | | LAYER, TYPE II; CDB2, OMIM_236730: UROFACIAL SYNDROME; |
| | | | | | | UFS, OMIM_609041: SPASTIC PARAPLEGIA 27, AUTOSOMAL |
| | | | | | | RECESSIVE; SPG27, OMIM_608583: ATRIAL FIBRILLATION, |
| | | | | | | FAMILIAL, 2; ATFB2, OMIM_605526: ALZHEIMER DISEASE 6, |
| | | | | | | OMIM_608176: AUTOIMMUNE THYROID DISEASE, SUSCEPTIBILITY |
| | | | | | | TO, 4, OMIM_166760: OTITIS MEDIA, SUSCEPTIBILITY TO |

TABLE 8-11

| | | | | | | |
|---|---|---|---|---|---|---|
| FLJ25460 | 59 | 19p13.3 | 1733076 | 1763275 | − | OMIM_181800: SCOLIOSIS, IDIOPATHIC; IS1, OMIM_602477: |
| | | | | | | FEBRILE CONVULSIONS, FAMILIAL, 2; FEB2, OMIM_145981: |
| | | | | | | HYPOCALCIURIC HYPERCALCEMIA, FAMILIAL, TYPE II; HHC2, |
| | | | | | | OMIM_601846: VACUOLAR NEUROMYOPATHY, OMIM_609306: |
| | | | | | | SPINOCEREBELLAR ATAXIA 26; SCA26, OMIM_108725: |
| | | | | | | ATHEROSCLEROSIS SUSCEPTIBILITY; ATHS, OMIM_606674: |
| | | | | | | INFLAMMATORY BOWEL DISEASE 6; IBD6, OMIM_607508: |
| | | | | | | MIGRAINE WITH OR WITHOUT AURA, SUSCEPTIBILITY TO, 5, |
| | | | | | | OMIM_605364: PSORIASIS SUSCEPTIBILITY 6, OMIM_125630: |
| | | | | | | DERMODISTORTIVE URTICARIA; DDU, OMIM_600209: |
| | | | | | | EXOSTOSES, MULTIPLE, TYPE III; EXT3, OMIM_120050: |
| | | | | | | COXSACKIEVIRUS B3 SUSCEPTIBILITY; CXB3S |
| FLJ26806 | 60 | 2q37.3 | 238489032 | 238533447 | + | OMIM_600430: BRACHYDACTYLY-MENTAL RETARDATION |
| | | | | | | SYNDROME, OMIM_600430: BRACHYDACTYLY-MENTAL |
| | | | | | | RETARDATION SYNDROME, OMIM_607688: PARKINSON DISEASE |
| | | | | | | 11; PARK11, OMIM_606053: AUTISM, SUSCEPTIBILITY TO, |
| | | | | | | 5; AUTS5, OMIM_606963: PULMONARY DISEASE, CHRONIC |
| | | | | | | OBSTRUCTIVE, SEVERE EARLY-ONSET |
| FLJ43911 | 61 | 20p12.1 | 13924015 | 15981839 | + | OMIM_608696: GLAUCOMA 1, OPEN ANGLE, K; GLC1K, |
| | | | | | | OMIM_608559: BODY MASS INDEX QUANTITATIVE TRAIT LOCUS |
| | | | | | | ON CHROMOSOME NO 20, IN CHILDREN, OMIM_607116: |
| | | | | | | ALZHEIMER DISEASE 8, OMIM_605804: DERMATITIS, ATOPIC, |
| | | | | | | 3; ATOD3, OMIM_605387: CATARACT, POSTERIOR POLAR, 3 |
| FLJ44715 | 62 | 10q22.2 | 75174138 | 75205980 | + | OMIM_604185: FACIAL PARESIS, HEREDITARY, CONGENITAL; |
| | | | | | | HCFP2, OMIM_609041: SPASTIC PARAPLEGIA 27, AUTOSOMAL |
| | | | | | | RECESSIVE; SPG27, OMIM_608583: ATRIAL FIBRILLATION, |
| | | | | | | FAMILIAL, 2; ATFB2, OMIM_605526: ALZHEIMER DISEASE 6, |
| | | | | | | OMIM_608176: AUTOIMMUNE THYROID DISEASE, SUSCEPTIBILITY |
| | | | | | | TO, 4, OMIM_166760: OTITIS MEDIA, SUSCEPTIBILITY TO |
| FLJ90031 | 63 | 17q21.2 | 37807994 | 37829061 | − | OMIM_609378: AUTISM, SUSCEPTIBILITY TO, 6; AUTS6, |
| | | | | | | OMIM_221820: GLIOSIS, FAMILIAL PROGRESSIVE SUBCORTICAL, |
| | | | | | | OMIM_608474: MYOPIA 5, OMIM_601363: WILMS TUMOR 4, |
| | | | | | | OMIM_161000: NAEGELI SYNDROME, OMIM_603918: HYPERTENSION, |
| | | | | | | ESSENTIAL, SUSCEPTIBILITY TO, 1, OMIM_602723: PSORIASIS |
| | | | | | | SUSCEPTIBILITY 2; PSORS2 |

Other examples of possible diseases or conditions are diseases or conditions accompanied by abnormalities at expression sites of target gene Y, or in tissues from which the source library for target gene Y is derived. The expression sites and tissues can easily be searched by, for example, inputting H-Inv cDNA ID numbers or H-Inv locus ID numbers in H-Inv DB, whereby those skilled in the art are able to postulate the diseases or conditions.

Still other examples of possible diseases or conditions are diseases or conditions mediated by genes that are homologous to target gene Y or a gene downstream thereof. Those skilled in the art are able to postulate such diseases or conditions by identifying homologous genes by homology search, and then extensively investigating the diseases or conditions involved by the homologous genes by a commonly known method.

The target proteins and target genes of the present invention are useful for, for example, the development of drugs for specified diseases or conditions, or the development of investigational reagents for the diseases or conditions.

### 2. Screening Methods and Products Obtained by the Methods

The present invention provides screening methods for bioactive substances, each of which comprises determining whether or not a test substance is capable of regulating the expression or function of a target protein for the bioactive substance or a gene that encodes the protein (hereinafter sometimes referred to as “target protein Y” or “target gene Y” as required), and a product thereof. The screening methods of the present invention can be roughly divided into two types, from the viewpoint of the kind of bioactive substance screened: screening methods for substances capable of regulating an action associated with a bioactive substance X, and screening methods for substances capable of regulating a function associated with a target protein Y. The screening methods of the present invention can also be performed in vitro, in vivo or in silico. The individual screening methods are hereinafter described in detail.

2.1. Screening Methods for Substances Capable of Regulating an Action Associated with a Bioactive Substance X (Screening Method I)

The present invention provides screening methods for substances capable of regulating an action associated with a bioactive substance X, each of which comprises determining whether or not a test substance is capable of regulating the expression or function of a target protein Y.

The screening methods of this type are generically referred to as “screening method I” as required.

Screening method I can be roughly divided into two types: a screening method for a substance capable of regulating an action associated with a bioactive substance X, which comprises determining whether or not a test substance is capable of regulating the expression or function of a target protein Y, and selecting a test substance capable of regulating the expression or function of a target protein Y (screening method Ia), and a screening method for a substance capable of regulating an action associated with a bioactive substance X (particularly an action associated with a known target molecule), which comprises determining whether or not a test substance is capable of regulating the expression or function of a target protein Y, and selecting a test substance that is incapable of regulating the expression or function of a target protein Y (screening method Ib). Screening method Ia can be useful for the development of regulators of diseases or conditions associated with bioactive substance X and the like. Screening method Ib can be useful for the development of drugs capable of regulating an action associated with a known target molecule, and showing decreased adverse effects of bioactive substance X and the like.

2.1.1. Screening Method for Substances Capable of Regulating an Action Associated with a Bioactive Substance X, Which Comprises Selecting a Test Substance Capable of Regulating the Expression or Function of a Target Protein Y (Screening Method Ia)

The present invention provides a screening method for substances capable of regulating an action associated with a bioactive substance X, which comprises determining whether or not a test substance is capable of regulating the expression or function of a target protein Y, and selecting a test substance capable of regulating the expression or function of a target protein Y.

The test substance subjected to this screening method may be any known compound and new compound; examples include nucleic acids, saccharides, lipids, proteins, peptides, organic small compounds, compound libraries prepared using combinatorial chemistry technique, random peptide libraries prepared by solid phase synthesis or the phage display method, or natural components derived from microorganisms, animals, plants, marine organisms and the like, and the like. The test substance may be a labeled supply or a non-labeled supply, or a mixture of a labeled supply and a non-labeled supply mixed in a specified ratio. The labeling substance is the same as described above.

In one embodiment, screening method Ia comprises the following steps (a), (b) and (c):
(a) a step for bringing the test substance into contact with target protein Y;
(b) a step for measuring the functional level of the protein in the presence of the test substance, and comparing this functional level with the functional level of the protein in the absence of the test substance;
(c) a step for selecting a test substance that alters the functional level of the protein on the basis of the result of the comparison in step (b) above.

The methodology comprising the above-described steps (a) to (c) is referred to as “methodology I” as required.

In step (a) of methodology I, a test substance is brought into contact with target protein Y. Contact of the test substance with the protein can be performed by contact of isolated target protein Y and the test substance in solution, or contact of cells or tissue capable of expressing target protein Y and the test substance.

Target protein Y can be prepared by a method known per se. For example, target protein Y can be isolated and purified from the above-described expression tissue. However, to prepare target protein Y quickly, easily, and in large amounts, and to prepare human target protein Y, it is preferable to prepare a recombinant protein by gene recombination technology. The recombinant protein may be prepared using a cell system or a cell-free system.

The cells capable of expressing target protein Y can be any cells that express target protein Y; examples include cells derived from the tissue in which target protein Y is expressed, cells transformed with a target protein Y expression vector and the like. Those skilled in the art are able to easily identify or prepare these cells; useful cells include primary culture cells, cell lines derivatively prepared from the primary culture cells, commercially available cell lines, cell lines available from cell banks, and the like. As the tissue capable of expressing target protein Y, the above-described expression tissues can be used.

In step (b) of methodology I, the functional level of the protein in the presence of the test substance is measured. A measurement of the functional level can be performed according to the kind of protein by a method known per se. For example, provided that target protein Y is a transcription factor, a substance that regulates a function associated with a target protein Y can be screened by performing a reporter assay using target protein Y and a transcription regulatory region to which it binds.

Provided that target protein Y is an enzyme, the functional level can also be measured on the basis of a change in the catalytic activity of the enzyme. The catalytic activity of the enzyme can be measured by a method known per se using a substrate, coenzyme and the like chosen as appropriate according to the kind of enzyme.

Furthermore, provided that target protein Y is a membrane protein (e.g., receptors, transporters), the functional level can be measured on the basis of a change in a function of the membrane protein. For example, provided that target protein Y is a receptor, a screening method of the present invention can be performed on the basis of an intracellular event mediated by the receptor (e.g., inositol phospholipid production, intracellular pH change, intracellular behavior of ions such as calcium ion and chlorine ion). Provided that target protein Y is a transporter, a screening methods of the present invention can be performed on the basis of a change in the intracellular concentration of a substrate for the transporter.

The functional level may also be measured on the basis of the functional level of target protein Y to each isoform (e.g., splicing variant) or the isoform-isoform functional level ratio, rather than on the basis of the total functional level of target protein Y.

Next, the functional level of target protein Y in the presence of the test substance is compared with the functional level of target protein Y in the absence of the test substance. This comparison of the functional levels is preferably performed on the basis of the presence or absence of a significant difference. Although the functional level of target protein Y in the absence of the test substance may be measured prior to, or simultaneously with, the measurement of the functional level of target protein Y in the presence of the test substance, it is preferable, from the viewpoint of experimental accuracy and reproducibility, that the functional level be measured simultaneously.

In step (c) of methodology I, a test substance that alters the functional level of the protein is selected. The test substance that alters the functional level of the protein is capable of promoting or suppressing a function of a target protein Y. The test substance thus selected can be useful for the regulation of a disease or condition associated with bioactive substance X.

Methodology I may be performed not only in the presence of target protein Y but also with a coupling factor thereof. For example, when a target protein Y inhibitory factor is used in combination as the coupling factor of target protein Y, a substance that interferes with the interaction between target protein Y and the coupling factor is considered to be capable of promoting a function of a target protein Y. When a target protein Y activation factor is used in combination as the coupling factor of target protein Y, a substance that interferes with the interaction between target protein Y and the coupling factor is considered to be capable of suppressing a function of a target protein Y. Hence, it is also beneficial to perform methodology I in the presence of a coupling factor of target protein Y.

In another embodiment, screening method Ia comprises the following steps (a), (b) and (c):
(a) a step for bringing the test substance into contact with cells enabling a measurement of the expression of target protein Y or a gene that encodes the protein;
(b) a step for measuring the expression level in the cells in contact with the test substance, and comparing this expression level with the expression level in control cells not in contact with the test substance;
(c) a step for selecting a test substance that regulates the expression level on the basis of the result of the comparison in step (b) above.

The methodology comprising the above-described steps (a) to (c) is referred to as “methodology II” as required.

In step (a) of methodology II, a test substance is brought into contact with cells enabling a measurement of the expression of target protein Y. Contact of the test substance with the cells enabling a measurement of the expression of target protein Y can be performed in culture medium.

“Cells enabling a measurement of the expression of target protein Y or a gene that encodes the protein (referred to as “target gene Y” as required)” refers to cells enabling a direct or indirect evaluation of the expression level of a product of target gene Y, for example, a transcription product or translation product (i.e., protein). The cells enabling a direct evaluation of the expression level of a product of target gene Y can be cells capable of naturally expressing target gene Y, whereas the cells enabling an indirect evaluation of the expression level of a product of target gene Y can be cells enabling a reporter assay on the target gene Y transcription regulatory region.

The cells capable of naturally expressing target gene Y can be any cells that potentially express target gene Y; examples include cells showing permanent expression of target gene Y, cells that express target gene Y under inductive conditions (e.g., drug treatment) and the like. Those skilled in the art are able to easily identify these cells; useful cells include primary culture cells, cell lines induced from the primary culture cells, commercially available cell lines, cell lines available from cell banks, and the like.

The cells enabling a reporter assay on the target gene Y transcription regulatory region are cells incorporating the target gene Y transcription regulatory region and a reporter gene functionally linked to the region. The target gene Y transcription regulatory region and reporter gene are inserted in an expression vector.

The target gene Y transcription regulatory region may be any region enabling the control of the expression of target gene Y; examples include a region from the transcription initiation point to about 2 kbp upstream thereof, and a region consisting of a base sequence wherein one or more bases are deleted, substituted or added in the base sequence of the region, and that is capable of controlling the transcription of target gene Y, and the like.

The reporter gene may be any gene that encodes a detectable protein or enzyme; examples include the GFP (green fluorescent protein) gene, GUS (β-glucuronidase) gene, LUS (luciferase) gene, CAT (chloramphenicol acetyltransferase) gene and the like.

The cells transfected with the target gene Y transcription regulatory region and a reporter gene functionally linked to the region are not subject to limitation, as long as they enable an evaluation of the target gene Y transcription regulatory function, that is, as long as they enable a quantitative analysis of the expression level of the reporter gene. However, the cells transfected are preferably cells capable of naturally expressing target gene Y because they are considered to express a physiological transcription regulatory factor for target gene Y, and to be more appropriate for the evaluation of the regulation of the expression of target gene Y.

The culture medium in which a test substance and cells enabling a measurement of the expression of target gene Y are brought into contact with each other is chosen as appropriate according to the kind of cells used and the like; examples include minimal essential medium (MEM) containing about 5 to 20% fetal bovine serum, Dulbecco's modified minimal essential medium (DMEM), RPMI1640 medium, 199 medium and the like. Culture conditions are also determined as appropriate according to the kind of cells used and the like; for example, the pH of the medium is about 6 to about 8, culture temperature is normally about 30 to about 40° C., and culture time is about 12 to about 72 hours.

In step (b) of methodology II, first, the expression level of target gene Y in the cells in contact with the test substance is measured. This measurement of expression level can be performed by a method known per se in view of the kind of cells used and the like.

For example, when cells capable of naturally expressing target gene Y are used as the cells enabling a measurement of the expression of target gene Y, the expression level can be measured by a method known per se with a product of target gene Y, for example, a transcription product or translation product, as the subject. For example, the expression level of a transcription product can be measured by preparing total RNA from the cells, and performing RT-PCR, Northern blotting and the like. The expression level of a translation product can also be measured by preparing an extract from the cells, and performing an immunological technique. Useful immunological techniques include radioisotope immunoassay (RIA), ELISA (Methods in Enzymol. 70: 419-439 (1980)), fluorescent antibody and the like.

On the other hand, when cells enabling a reporter assay on the target gene Y transcription regulatory region are used as the cells enabling a measurement of the expression of target gene Y, the expression level can be measured on the basis of the signal intensity of the reporter.

The expression level may also be measured on the basis of the expression level of target gene Y to each isoform (e.g., splicing variant) or the isoform-isoform expression ratio, rather than on the basis of the total functional level of target gene Y.

Next, the expression level of target gene Y in the cells in contact with the test substance is compared with the expression level of target gene Y in control cells not in contact with the test substance. This comparison of the expression levels is preferably performed on the basis of the presence or absence of a significant difference. Although the expression level of target gene Y in the control cells not in contact with the test substance may be measured prior to, or simultaneously with, the measurement of the expression level of target gene Y in the cells in contact with the test substance, it is preferable, from the viewpoint of experimental accuracy and reproducibility, that the expression level be measured simultaneously.

In step (c) of methodology II, a test substance that regulates the expression level of target gene Y is selected. The regulation of the expression level of target gene Y can be the promotion or suppression of the expression level. The test substance thus selected can be useful for the regulation of an action associated with a bioactive substance X.

Methodology II can further comprise (d) (i) a step for confirming that the selected test substance is capable of regulating, for example, promoting or suppressing, an action associated with a bioactive substance X (confirmation step), or (ii) a step for identifying the kind of action exhibited by the selected test substance (identification step). The confirmation step or identification step can be performed by, for example, administering the selected test substance to a normal animal, or to an animal with “a disease or condition associated with bioactive substance X” or model animal. According to this identification step, the kind of “action associated with a bioactive substance X” exhibited by the selected test substance can be determined, and whether or not the selected test substance can be used as either a drug or an investigational reagent, or both, and the kind of drug or investigational reagent to which the test substance is applicable can be confirmed.

In another embodiment, screening method Ia comprises the following steps (a), (b) and (c):
(a) a step for bringing the test substance into contact with target protein Y;
(b) a step for measuring the ability of the test substance to bind to the protein;
(c) a step for selecting a test substance capable of binding to the protein on the basis of the results of step (b) above.

The methodology comprising the above-described steps (a) to (c) is referred to as “methodology III” as required.

In step (a) of methodology III, a test substance is brought into contact with target protein Y. Contact of the test substance with the protein can be performed by mixing the test substance and the protein in solution.

Target protein Y can be prepared by a method known per se. For example, target protein Y can be isolated and purified from the above-described target gene Y expression tissue. However, to prepare target protein Y quickly, easily, and in large amounts, and to prepare human target protein Y, it is preferable to prepare a recombinant protein by gene recombination technology. The recombinant protein may be prepared using a cell system or a cell-free system.

In step (b) of methodology III, the ability of the test substance to bind to the protein is measured. “a binding ability” measured may be any one that enables an evaluation of the binding of the protein and the test substance; examples include binding amount, binding strength (including parameters such as affinity constant, binding rate constant, and dissociation rate constant), and binding mode (including dose-dependent binding).

A measurement of the binding ability can be performed by, for example, the SEC/MS (size exclusion chromatography/mass analysis) method (see Moy, F. J. et al., Anal. Chem., 2001, 73, 571-581). The SEC/MS method comprises (1) a step for adding a mixed multiplied compound standard to the purified protein, and then separating the free compound and the protein by SEC, and (2) an analytical step for identifying the bound compound contained in the protein fraction by MS. The SEC/MS method is advantageous in that the binding ability can be analyzed while both the protein and the test substance are in non-modified and non-immobilized state. In the SEC/MS method, not only the binding ability of the test substance to the protein, but also the dose dependency of the test substance in the binding to the protein and the like can be measured simultaneously.

A measurement of the binding ability can also be performed using a means for measurement based on surface plasmon resonance, for example, Biacore. Using Biacore, the binding and dissociation of a test substance to a protein immobilized on a chip are measured, and the measured values are compared with those obtained when a solution not containing the test substance is loaded on the chip. Subsequently, a test substance capable of binding to the protein is selected on the basis of the result for the binding and dissociation rate or binding amount. Biacore also enables simultaneous measurements of binding strength (e.g., K_{d} value) and the like, in addition to the binding ability of a test substance to a protein.

Other methods for measuring the binding ability include, for example, SPR-based methods or optical methods such as the quartz crystal microbalance (QCM) method, the dual polarization interferometer (DPI) method, and the coupled waveguide plasmon resonance method, immunoprecipitation, isothermal titration and differential scanning calorimetry, capillary electrophoresis, energy transfer, fluorescent analytical methods such as fluorescent correlation analysis, and structural analytical methods such as X-ray crystallography and nuclear magnetic resonance (NMR).

In measuring the binding ability, a target protein Y-binding substance can also be used as a control.

“A target protein Y-binding substance” is a compound capable of interacting directly with target protein Y or a mutated protein thereof, and can be, for example, a protein, a nucleic acid, a carbohydrate, a lipid, or a small organic compound. The target protein Y-binding substance can be preferably selected from trimethylcolchicic acid, acenocoumarol, paracetamol, acetohexamide, acetopromazine, actinomycin D, ajmaline, albendazole, alfuzosin, α-methyl-5-hydroxytryptamine, amoxapine, antipyrine, azithromycin, benzbromarone, benzethonium, benzydamine, berberine, bezafibrate, bicartamide, boldine, bromperidol, budesonide, bupivacaine, buspirone, cefazolin, celestine blue, cephaeline, chlordiazepoxide, chlorogenic acid, chlorothiazide, chromomycin A3, ciclopirox, cisapride, clarithromycin, clemizole, clenbuterol, clobetasone, clofazimine, clofilium, clomiphene, clopamide, colchicine, colistin, conessine, coniine (DL), coralyne, cyclobenzaprine, cyclopentolate, cyclosporine A, diclofenac, dichlorphenamide, diflunisal, dihydrostreptomycin, diperodon, difenidol, dipyridamole, dizocilpine, DO897/99, domperidone, dopamine, doxazosin, doxycycline, eburnamonine, etodolac, fenbendazole, fenbufen, fenoprofen, flumequine, flupentixol, fluphenazine, fluvoxamine, furazolidone, gabapentin, GBR12909, glibenclamide, glipizide, gramicidin, guanfacine, harmol, hydroflumethiazide, hydroxychloroquine, hydroxytacrine(R,S), ifosfamide, iobenguane, iproniazid, isoxicam, isradipine, josamycin, ketoprofen, 3-hydroxykynurenine, leuprolide, L-thyroxine, lidoflazine, α-lobeline (−), loperamide, maprotiline, mebendazole, meclofenamic acid, metanephrine (D,L), metaproterenol, metergotamine, methimazole, methoxamine, methoxy-6-harmalan, mifepristone, minaprine, minocycline, misoprostol, molsidomine, moroxydine, moxalactam, mupirocin, nefopam, nicardipine, nimesulide, norharman, oxytocin, paroxetine, perhexiline, phenformin, pimethixene, piperlongumine, pirenzepine, probenecid, procaine, propranolol, protriptyline, pyrilamine, quercetin, quinacrine, quinine, rescinnamine, risperidone, ritodrine, saquinavir, scoulerine, sulfadimethoxine, sulfaphenazole, syrosingopine, tamoxifen, terconazole, thioproperazine, thiothixene(cis), tobramycin, tolbutamide, trifluoperazine, trimetazidine, viloxazine, xylazine, acetylsalicylsalicylic acid, nimetazepam, clobazam, alimemazine, tranilast, ebastine, pranlukast, methyclothiazide, alacepril, clinofibrate, acetylcysteine, buformin, terguride, stanozolol, mestanolone, pantethine, limaprost, sarpogrelate, argatroban, fludroxycortide, sulfadoxine, ubenimex, celecoxib, 6-furfurylaminopurine, solasodine, gossypol, fluorocurarine, pempidine, nitrarine, promazine, sulfabenzamide, althiazide, α-ergocryptine, ebselen, furaltadone, pyrithyldione, benzthiazide, levobunolol, raloxifene, luteolin, valdecoxib, carboprost, gabexate, and derivatives thereof capable of binding to target protein Y (determined according to the kind of bioactive substance X) (described later), and salts thereof.

Although the salts may be any salts, pharmaceutically acceptable salts are preferable; examples include salts with inorganic bases (e.g., alkali metals such as sodium and potassium; alkaline earth metals such as calcium and magnesium; aluminum, ammonium), salts with organic bases (e.g., trimethylamine, triethylamine, pyridine, picoline, ethanolamine, diethanolamine, triethanolamine, dicyclohexylamine, N,N-dibenzylethylenediamine), salts with inorganic acids (e.g., hydrochloric acid, hydrobromic acid, nitric acid, sulfuric acid, phosphoric acid), salt with organic acids (e.g., formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid), salts with basic amino acids (e.g., arginine, lysine, ornithine) or salts with acidic amino acids (e.g., aspartic acid, glutamic acid) and the like.

Furthermore, the binding ability may also be measured on the basis of the binding ability of target protein Y to each isoform (e.g., splicing variant) or the isoform-isoform binding ability ratio, rather than on the basis of the total binding ability of target protein Y.

The binding ability can also be measured in silico. For example, a measurement of the binding ability can be performed on the basis of SBDD (structure-based drug design: SBDD) or CADD (computer-aided drug design). Examples of such screening include virtual screening, de novo design, pharmacophore analysis, QSAR (quantitative structure activity relationship) and the like. If information on the steric structure of the protein or the target site of the protein is required during such screening, the information on the steric structure is used, provided that the steric structure is known by a structural analytical technique such as NMR, X-ray crystallographic analysis, or synchrotron radiation analysis. If the steric structure is unknown, information obtained by a structural estimation method such as the homology method or the threading method is used. In virtual screening, a program known per se can be used; examples of the program include DOCK (Kuntz, I. D. et al., Science, 1992, 257, 1078), Gold (Jones, G. et al., J. Mol. Biol., 1995, 245, 43), FlexX (Rarey, M. et al., J. Mol. Biol., 1996, 261, 470), AutoDock (Morris, G. M. et al., J. Comput. Chem., 1998, 19, 1639), ICM (Abagyan, R. A. et al., J. Comput. Chem., 1994, 15, 488) and the like.

In step (c) of methodology III, a test substance capable of binding to target protein Y is selected. The test substance capable of binding to the protein is capable of promoting or suppressing a function of a target protein Y. Thus, the selected test substance can be useful for the regulation of a disease or condition associated with bioactive substance X.

Methodology III can further comprise (d) (i) a step for confirming that the selected test substance is capable of regulating, for example, promoting or suppressing, an action associated with a bioactive substance X (confirmation step), or (ii) a step for identifying the kind of action exhibited by the selected test substance (identification step). The confirmation step or identification step can be performed by, for example, administering the selected test substance to a normal animal, or to an animal with “a disease or condition associated with bioactive substance X” or model animal. According to this identification step, the kind of “action associated with a bioactive substance X” possessed by the selected test substance can be determined, and whether or not the selected test substance can be used as either a drug or an investigational reagent, or both, and the kind of drug or investigational reagent to which the test substance is applicable can be confirmed.

In still another mode of embodiment, screening method Ia comprises the following steps (a), (b) and (c):
(a) a step for bringing the test substance and a target protein Y-binding substance into contact with target protein Y;
(b) a step for measuring the ability of the target protein Y-binding substance to bind to the protein in the presence of the test substance, and comparing this binding ability with the ability of the target protein Y-binding substance to bind to the protein in the absence of the test substance;
(c) a step for selecting a test substance that alters the ability of the target protein Y-binding substance to bind to the protein on the basis of the result of the comparison in step (b) above.

The methodology comprising the above-described steps (a) to (c) is referred to as “methodology IV” as required.

In step (a) of methodology IV, both a test substance and a target protein Y-binding substance are brought into contact with target protein Y. Contact of the test substance and the target protein Y-binding substance with the protein can be performed by mixing the test substance, the target protein Y-binding substance, and the protein in solution. The order of bringing the test substance and target protein Y-binding substance into contact with the protein is not subject to limitation; one of them may be brought into contact with the protein at a time lag or at the same time.

Target protein Y can be prepared by a method known per se. For example, preparation of the protein can be performed by a method described in methodology III above.

The target protein Y-binding substance may be a labeled supply or a non-labeled supply, or a mixture of a labeled supply and a non-labeled supply mixed in a specified ratio. The labeling substance is the same as described above.

In step (b) of methodology IV, first, the ability of the target protein Y-binding substance to bind to the protein is measured in the presence of the test substance. “A binding ability” measured may be any one that enables an evaluation of the binding of the protein and the test substance; examples include binding amount, binding strength (including parameters such as affinity constant, binding rate constant, and dissociation rate constant), and binding mode (including dose-dependent binding).

A measurement of the binding ability can be performed using, for example, a labeled target protein Y-binding substance. The target protein Y-binding substance bound to the protein and the unbound target protein Y-binding substance may be separated before measuring the binding ability. More specifically, a measurement of the binding ability can be performed in the same manner as methodology III.

The binding ability may also be measured on the basis of the binding ability of target protein Y to each isoform (e.g., splicing variant) or the isoform-isoform binding ability ratio, rather than on the basis of the total amount of target protein Y bound.

Next, the binding ability of the target protein Y-binding substance to the protein in the presence of the test substance is compared with the binding ability of the target protein Y-binding substance to the protein in the absence of the test substance. This comparison of the binding abilities is preferably performed on the basis of a significant difference. Although the binding ability of the target protein Y-binding substance to the protein in the absence of the test substance may be measured prior to, or simultaneously with, the measurement of the binding ability of the target protein Y-binding substance to the protein in the presence of the test substance, it is preferable, from the viewpoint of experimental accuracy and reproducibility, that the binding ability be measured simultaneously.

In step (c) of methodology IV, a test substance that alters the ability of the target protein Y-binding substance to bind to the protein is selected. The change in the binding ability can be, for example, a reduction or increase of binding ability, with preference given to a reduction of binding ability. Hence, the selected test substance can be useful for the regulation of an action associated with a bioactive substance X.

Methodology IV can further comprise (d) (i) a step for confirming that the selected test substance is capable of regulating, for example, promoting or suppressing, an action associated with a bioactive substance X (confirmation step), or (ii) a step for identifying the kind of action exhibited by the selected test substance (identification step). The confirmation step or identification step can be performed by, for example, administering the selected test substance to a normal animal or an animal with “a disease or condition associated with bioactive substance X” or model animal. According to this identification step, the kind of “action associated with a bioactive substance X” exhibited by the selected test substance can be determined, and whether or not the selected test substance can be used as either a drug or an investigational reagent, or both, and the kind of drug or investigational reagent to which the test substance is applicable can be confirmed.

Screening method Ia can also be performed using an animal. Examples of the animal include mammals such as mice, rats, hamsters, guinea pigs, rabbits, dogs, and monkeys, and birds such as chickens. When a screening method of the present invention is performed using an animal, for example, a test substance that regulates the expression level of target gene Y can be selected.

Screening method Ia can also be performed by various methodologies suitable to the kind of target gene Y. For example, provided that target gene Y is a gene for an intracellularly localized factor, screening method I can be performed on the basis of a change in the intracellular localization of target protein Y. The amount of target protein Y localized in a specified organelle can be measured by a method known per se. For example, target gene Y, previously fused with a gene that encodes a fluorescent protein, such as the GFP gene, is introduced to an appropriate cell and cultured in culture medium in the presence of a test substance. Next, a fluorescence signal in the specified organelle is examined using a confocal microscope, and this signal is compared with the fluorescence signal in the absence of the test substance in the same organelle. The amount of target protein Y localized in the specified organelle can also be measured by immunostaining using an antibody against target protein Y.

Furthermore, provided that target gene Y is a gene for a soluble (secretory) factor, screening method Ia can be performed on the basis of a change in the blood concentration of the factor in the animal. Administration of the test substance to the animal, blood drawing from the animal, and the measurement of the blood concentration of the factor can be performed by a method known per se.

Screening method Ia enables screening of a substance capable of regulating an action associated with a bioactive substance X. Hence, screening method Ia is useful for the development of a prophylactic or therapeutic agent for a disease or condition associated with bioactive substance X, an investigational reagent for the disease or the condition, and the like.

2.1.2. Screening Method for Substances Capable of Regulating an Action Associated with a Bioactive Substance X, Which Comprises Selecting a Test Substance Incapable of Regulating the Expression or Function of a Target Protein Y (Screening Method Ib)

The present invention provides a screening method for test substances capable of regulating an action associated with a bioactive substance X (particularly an action associated with a known target molecule), which comprises determining whether or not a test substance is capable of regulating the expression or function of a target protein Y, and selecting a test substance incapable of regulating the expression or function of a target protein Y.

Screening method Ib can be performed in the same manner as methodologies I to IV except that a test substance that does not cause a change or does not have the binding ability or regulatory capacity in step (c) of the above-described methodologies I to IV is selected.

In screening method Ib, the test substance used can be one capable of regulating the expression or function of a known target molecule. Hence, screening method Ib can be used in combination with a screening method for substances capable of regulating an action associated with a known target molecule, which comprises determining whether or not the test substance is capable of regulating the expression or function of the known target molecule. The screening method for substance's capable of regulating an action associated with a known target molecule can be performed in the same manner as the above-described screening method Ia.

Screening method Ib enables the development of drugs capable of regulating an action associated with a known target molecule, and showing decreased adverse effects of bioactive substance X. Hence, screening method Ib is useful for the improvement of existing drugs capable of regulating an action associated with a known target molecule and the like.

2.2. Screening Method for Substances Capable of Regulating a Function Associated with Target Protein Y (Screening Method II)

The present invention provides a screening method for substances capable of regulating a function associated with a target protein Y, which comprises comparing the ability of a test substance to bind to the target protein Y or the action associated with the test compound, with the ability of a bioactive substance X to bind to the target protein Y or the action associated with the bioactive substance.

This screening method is referred to as “screening method II” as required.

In one embodiment, screening method II comprises the following steps (a), (b) and (c):
(a) a step for bringing the test substance into contact with target protein Y;
(b) a step for measuring the functional level of the protein in the presence of the test substance, and comparing this functional level with the functional level of the protein in the presence of bioactive substance X;
(c) a step for selecting a test substance that alters the functional level of the protein on the basis of the result of the comparison in step (b) above.

The methodology comprising the above-described steps (a) to (c) is the same as methodology I except that the reference control for step (b) is not “the functional level of target protein Y in the absence of the test substance” but “the functional level of target protein Y in the presence of bioactive substance X”.

In another embodiment, screening method II comprises the following steps (a), (b) and (c):
(a) a step for bringing the test substance and cells enabling a measurement of the expression of target protein Y or a gene that encodes the protein into contact with each other;
(b) a step for measuring the expression level in the cells in contact with the test substance, and comparing this expression level with the expression level in control cells in contact with bioactive substance X;
(c) a step for selecting a test substance that regulates the expression level on the basis of the result of the comparison in step (b) above.

The methodology comprising the above-described steps (a) to (c) is the same as methodology II except that the reference control for step (b) is not “the expression level in control cells not in contact with the test substance” but “the expression level in control cells in contact with bioactive substance X”.

In still another mode of embodiment, screening method II comprises the following steps (a), (b) and (c):
(a) a step for bringing the test substance into contact with target protein Y;
(b) a step for measuring the ability of the test substance to bind to the protein, and comparing this binding ability with the ability of bioactive substance X to bind to the protein;
(c) a step for selecting a test substance capable of binding to the protein on the basis of the result of step (b) above.

The methodology comprising the above-described steps (a) to (c) is the same as methodology III except that the reference control for step (b) is “the ability of target protein Y to bind to bioactive substance X”.

Screening method II enables, for example, screening of substances capable of regulating a function associated with a target protein Y, or probes for target protein Y, and the like. Hence, screening method II is useful for the screening of prophylactic or therapeutic agents for diseases or conditions associated with target gene Y, and screening of investigational reagents for the diseases or conditions, and the like.

### 2.3. Products Obtained by Screening Methods

The present invention provides products obtained by the above-described screening methods, for example, screening methods I and II.

A product provided by a screening method of the present invention can be a substance obtained by a screening method of the present invention, and a bioactivity regulator comprising a substance obtained by the screening method (described later).

A product provided by a screening method of the present invention is useful for, for example, the prevention or treatment of a disease or condition associated with bioactive substance X, or a disease or condition associated with target gene Y, or as an investigational reagent for the disease or the condition, and the like.

### 3. Regulators

The present invention provides bioactivity regulators each comprising a substance that regulates the expression or function of a target gene for a bioactive substance. The regulators of the present invention can be roughly divided into two types from the viewpoint of the bioactivity regulated: regulators of actions associated with bioactive substance X, and regulators of functions associated with target protein Y. The individual regulators are hereinafter described in detail.

3.1. Regulators of Actions Associated with Bioactive Substance X (Regulator I)

The present invention provides a type of regulators of actions associated with bioactive substance X, each of which comprises a substance that regulates the expression or function of target gene Y.

The regulators of this type are generically referred to as “regulator I” as required.

The substance that regulates the expression or function of target gene Y can be, for example, a substance that suppresses the expression of target gene Y. The expression refers to a state in which a target gene Y translation product is produced and is localized at the action site thereof in a functional condition. Hence, the substance that suppresses the expression may be one that acts in any stage of gene transcription, post-transcriptional regulation, translation, post-translational modification, localization and protein folding and the like.

Specifically, the substance that suppresses the expression of target gene Y is exemplified by transcription suppressor, RNA polymerase inhibitor, RNA decomposing enzyme, protein synthesis inhibitor, nuclear translocation inhibitor, protein decomposing enzyme, protein denaturant and the like; to minimize the adverse effects on other genes and proteins expressed in the cells, it is important that the substance that suppresses the expression of target gene Y be capable of specifically acting on the target molecule.

An example of the substance that suppresses the expression of target gene Y is an antisense nucleic acid to a transcription product of target gene Y, specifically mRNA or initial transcription product. “An antisense nucleic acid” refers to a nucleic acid that consists of a base sequence capable of hybridizing to the target mRNA (initial transcription product) under physiological conditions for cells that express target mRNA (initial transcription product), and capable of inhibiting the translation of the polypeptide encoded by the target mRNA (initial transcription product) in a hybridized state. The kind of antisense nucleic acid may be DNA or RNA, or a DNA/RNA chimera. Because a natural type antisense nucleic acid easily undergoes degradation of the phosphoric acid diester bond thereof by a nucleic acid decomposing enzyme present in the cells, an antisense nucleic acid of the present invention can also be synthesized using a modified nucleotide of the thiophosphate type (P═O in phosphate linkage replaced with P═S), 2′-O-methyl type and the like which are stable to decomposing enzymes. Other important factors for the designing of antisense nucleic acid include increases in water-solubility and cell membrane permeability and the like; these can also be cleared by choosing appropriate dosage forms such as those using liposome or microspheres.

The length of antisense nucleic acid is not subject to limitation, as long as the antisense nucleic acid is capable of specifically hybridizing to the transcription product of target gene Y; the antisense nucleic acid may be of a sequence complementary to a sequence of about 15 bases for the shortest, or the entire sequence of the mRNA (initial transcription product) for the longest. Considering the ease of synthesis, antigenicity and other issues, for example, oligonucleotides consisting of about 15 bases or more, preferably about 15 to about 30 bases, can be mentioned.

The target sequence for the antisense nucleic acid may be any sequence that inhibits the translation of target gene Y or a functional fragment thereof by being hybridized to the antisense nucleic acid, and may be the entire sequence or a partial sequence of mRNA, or the intron moiety of the initial transcription product; when an oligonucleotide is used as the antisense nucleic acid, it is desirable that the target sequence be located between the 5′ terminus of the mRNA of target gene Y and the C terminus of the coding region thereof.

Furthermore, the antisense nucleic acid may be not only capable of hybridizing to a transcription product of target gene Y to inhibit its translation, but also binding to target gene Y in the form of double-stranded DNA to form a triple-strand (triplex) and inhibit the transcription to mRNA.

Another example of the substance that suppresses the expression of target gene Y is a ribozyme capable of specifically cleaving a transcription product of target gene Y, specifically mRNA or initial transcription product in the coding region (including the intron portion in the case of initial transcription product). “A ribozyme” refers to an RNA possessing enzyme activity to cleave nucleic acids. Because it has recently been shown that an oligo-DNA having the base sequence of the enzyme activity site also possesses nucleic acid cleavage activity, this term is herein used to mean a concept including DNA, as long as sequence specific nucleic acid cleavage activity is possessed. The most versatile ribozyme includes self-splicing RNAs found in infectious RNAs such as those of viroid and virosoid, and hammerhead type, hairpin type and the like are known. When ribozyme is used in the form of an expression vector comprising a DNA that encodes the same, a hybrid ribozyme wherein a sequence modified from tRNA is further linked to promote localization to cytoplasm may be used [Nucleic Acids Res., 29(13): 2780-2788 (2001)].

A still another example of the substance that suppresses the expression of target gene Y is a decoy nucleic acid. A decoy nucleic acid refers to a nucleic acid molecule that mimics a region to which a transcription regulatory factor binds; the decoy nucleic acid, which is the substance that suppresses the expression of target gene Y, can be a nucleic acid molecule that mimics a region to which a transcription activation factor for target gene Y binds.

Examples of the decoy nucleic acid include oligonucleotides modified to make them unlikely to undergo degradation in a body, such as oligonucleotides having a thio-phosphodiester bond wherein an oxygen atom in the phosphodiester bond moiety is replaced with a sulfur atom (S-oligo), or oligonucleotides wherein the phosphodiester bond is replaced with an uncharged methyl phosphate group, and the like. Although the decoy nucleic acid may completely match with the region to which a transcription activation factor binds, the degree of matching may be such that the transcription activation factor is capable binding to target gene Y is retained. The length of the decoy nucleic acid is not subject to limitation, as long as the transcription activation factor binds thereto. The decoy nucleic acid may comprise a repeat of the same region.

Still another example of the substance that suppresses the expression of target gene Y is a double-stranded oligo-RNA, i.e. siRNA, which is complementary to a partial sequence (including the intron portion in the case of an initial transcription product) in the coding region of a transcription product of target gene Y, specifically, the mRNA or initial transcription product. It has been known that so-called RNA interference (RNAi), which is a phenomenon that if short double stranded RNA is introduced into cells, mRNA complementary to the RNA is degraded, occurs in nematodes, insects, plants and the like; recently, it has been found that this phenomenon also occurs in animal cells [Nature, 411(6836): 494-498 (2001)], which is drawing attention as an alternative technique to ribozymes. The siRNA used may be internally synthesized as described below, and a commercially available one may be used.

An antisense oligonucleotide and ribozyme can be prepared by determining the target sequence for a transcription product of target gene Y, specifically the mRNA or initial transcription product on the basis of the cDNA sequence or genomic DNA sequence of target gene Y, and by synthesizing a sequence complementary thereto using a commercially available automated DNA/RNA synthesizer (Applied Biosystems Company, Beckman Instruments Company and the like). A decoy nucleic acid and siRNA can be prepared by synthesizing a sense strand and an antisense strand in an automated DNA/RNA synthesizer, respectively, denaturing the chains in an appropriate annealing buffer solution at about 90 to about 95° C. for about 1 minute, and then annealing the chains at about 30 to about 70° C. for about 1 to about 8 hours. A longer double-stranded polynucleotide can be prepared by synthesizing a complementary oligonucleotide chain in alternative overlaps, annealing them, and then ligating them with ligase.

Another example of the substance that suppresses the expression of target gene Y is an antibody against target protein Y. The antibody may be a polyclonal antibody or a monoclonal antibody, and can be prepared by a well-known immunological technique. The antibody may also be a fragment of an antibody (e.g., Fab, F(ab′)₂), or a recombinant antibody (e.g., single-chain antibody). Furthermore, the nucleic acid that encodes the antibody (one functionally linked to a nucleic acid having promoter activity) is also preferable as the substance that suppresses the expression of target gene Y.

The polyclonal antibody can be acquired by, for example, subcutaneously or intraperitoneally administering target protein Y or a fragment thereof (as required, may be prepared as a complex crosslinked to a carrier protein such as bovine serum albumin or KLH (keyhole limpet hemocyanin)) as the antigen, along with a commercially available adjuvant (e.g., Freund's complete or incomplete adjuvant) to an animal about 2 to 4 times at intervals of 2 to 3 weeks (the antibody titer of partially drawn serum has been determined by a known antigen-antibody reaction and its elevation has been confirmed in advance), collecting whole blood about 3 to about 10 days after final immunization, and purifying the antiserum. As the animal to receive the antigen, mammals such as rats, mice, rabbits, goat, guinea pigs, and hamsters can be mentioned.

The monoclonal antibody can be prepared by, for example, a cell fusion method (e.g., Takeshi Watanabe, Saibou Yugouhou No Genri To Monokuronaru Koutai No Sakusei, edited by Akira Taniuchi and Toshitada Takahashi, “Monokuronaru Koutai To Gan—Kiso To Rinsho—”, pages 2-14, Science Forum Shuppan, 1985). For example, the factor is administered subcutaneously or intraperitoneally along with a commercially available adjuvant to a mouse 2 to 4 times, and about 3 days after final administration, the spleen or lymph nodes are collected, and leukocytes are collected. These leukocytes and myeloma cells (e.g., NS-1, P3X63Ag8 and the like) are cell-fused to obtain a hybridoma that produces a monoclonal antibody against the factor. This cell fusion may be performed by the PEG method [J. Immunol. Methods, 81(2): 223-228 (1985)], or by the voltage pulse method [Hybridoma, 7(6): 627-633 (1988)]. A hybridoma that produces the desired monoclonal antibody can be selected by detecting an antibody that binds specifically to the antigen from the culture supernatant using a widely known EIA or RIA method and the like. Cultivation of the hybridoma that produces the monoclonal antibody can be performed in vitro, or in vivo such as in mouse or rat ascitic fluid, preferably in mouse ascitic fluid, and the antibody can be acquired from the culture supernatant of the hybridoma and the ascitic fluid of the animal, respectively.

However, in view of therapeutic efficacy and safety in humans, the antibody of the present invention may be a chimeric antibody or a humanized or human type antibody. The chimeric antibody can be prepared with reference to, for example, “Jikken Igaku (extra issue), Vol. 6, No. 10, 1988”, Japanese Patent Kokoku Publication No. HEI-3-73280 and the like. The humanized antibody can be prepared with reference to, for example, Japanese Patent Kohyo Publication No. HEI-4-506458, Japanese Patent Kokai Publication No. SHO-62-296890 and the like. The human antibody can be prepared with reference to, for example, “Nature Genetics, Vol. 15, p. 146-156, 1997”, “Nature Genetics, Vol. 7, p. 13-21, 1994”, Japanese Patent Kohyo Publication No. HEI-4-504365, International Patent Application Publication No. WO94/25585, “Nikkei Science, June issue, pp. 40 to 50, 1995”, “Nature, Vol. 368, pp. 856-859, 1994”, Japanese Patent Kohyo Publication No. HEI-6-500233 and the like.

The substance that regulates the expression or function of target gene Y can also be a substance that suppresses a function of target gene Y.

Although the substance that suppresses a function of target gene Y is not subject to limitation, as long as it is capable of interfering with an action of target gene Y, it is important that the substance be capable of specifically acting on the target molecule to minimize the adverse effect on other genes and proteins. Examples of the substance that specifically suppresses a function of target gene Y include a dominant negative mutant of target protein Y and a nucleic acid that encodes the mutant (one functionally linked to a nucleic acid having promoter activity).

A dominant negative mutant of target protein Y refers to a mutant having the activity thereof reduced as a result of mutagenesis to target protein Y. The dominant negative mutant can have the activity thereof indirectly inhibited by competing with natural target protein Y. The dominant negative mutant can be prepared by introducing a mutation to a nucleic acid that encodes target gene Y. Examples of the mutation include amino acid mutations in a functional domain that result in a decrease in the function responsible for the domain (e.g., deletion, substitution, and addition of one or more amino acids). The mutation can be introduced by a method known per se using PCR or a commonly known kit.

Provided that the substance that suppresses the expression of target gene Y is a nucleic acid molecule, the regulator of the present invention can have, as an active ingredient, an expression vector that encodes the nucleic acid molecule. In the expression vector, an oligonucleotide or polynucleotide that encodes the above-described nucleic acid molecule must be functionally linked to a promoter capable of exhibiting promoter activity in the cells of the recipient mammal. Any promoter capable of functioning in the recipient mammal can be used; examples include viral promoters such as the SV40-derived early promoter, cytomegalovirus LTR, Rous sarcoma virus LTR, MoMuLV-derived LTR, and adenovirus-derived early promoter, and mammalian structural protein gene promoters such as the β-actin gene promoter, PGK gene promoter, and transferrin gene promoter, and the like.

The expression vector preferably comprises a transcription termination signal, that is, a terminator region, downstream of the oligo (poly)nucleotide that encodes the nucleic acid molecule. The expression vector may further comprise a selection marker gene for selecting transformant cells (genes that confer resistance to drugs such as tetracycline, ampicillin, kanamycin, hygromycin, and phosphinothricin, gene that compensate for auxotrophic mutation, and the like).

Although the basic backbone vector used as the expression vector is not subject to limitation, vectors suitable for administration to mammals such as humans include viral vectors such as retrovirus, adenovirus, adeno-associated virus, herpesvirus, vaccinia virus, poxvirus, poliovirus, Sindbis virus, and Sendai virus. Adenovirus has advantageous features, including the very high efficiency of gene introduction and possibility of introduction to non-dividing cells. Because incorporation of the introduced gene to host chromosome is very rare, however, gene expression is transient, usually lasting for about 4 weeks. In view of the sustainability of therapeutic effect, it is also preferable to use adeno-associated virus, which offers relatively high gene transduction efficiency, which can be introduced to non-dividing cells, and which can be incorporated in chromosomes via a inverted terminal repeat sequence (ITR).

The substance that regulates the expression or function of target gene Y can be also trimethylcolchicic acid, acenocoumarol, paracetamol, acetohexamide, acetopromazine, actinomycin D, ajmaline, albendazole, alfuzosin, α-methyl-5-hydroxytryptamine, amoxapine, antipyrine, azithromycin, benzbromarone, benzethonium, benzydamine, berberine, bezafibrate, bicartamide, boldine, bromperidol, budesonide, bupivacaine, buspirone, cefazolin, celestine blue, cephaeline, chlordiazepoxide, chlorogenic acid, chlorothiazide, chromomycin A3, ciclopirox, cisapride, clarithromycin, clemizole, clenbuterol, clobetasone, clofazimine, clofilium, clomiphene, clopamide, colchicine, colistin, conessine, coniine (DL), coralyne, cyclobenzaprine, cyclopentolate, cyclosporine A, diclofenac, dichlorphenamide, diflunisal, dihydrostreptomycin, diperodon, difenidol, dipyridamole, dizocilpine, DO897/99, domperidone, dopamine, doxazosin, doxycycline, eburnamonine, etodolac, fenbendazole, fenbufen, fenoprofen, flumequine, flupentixol, fluphenazine, fluvoxamine, furazolidone, gabapentin, GBR12909, glibenclamide, glipizide, gramicidin, guanfacine, harmol, hydroflumethiazide, hydroxychloroquine, hydroxytacrine(R,S), ifosfamide, iobenguane, iproniazid, isoxicam, isradipine, josamycin, ketoprofen, 3-hydroxykynurenine, leuprolide, L-thyroxine, lidoflazine, α-lobeline (−), loperamide, maprotiline, mebendazole, meclofenamic acid, metanephrine (D,L), metaproterenol, metergotamine, methimazole, methoxamine, methoxy-6-harmalan, mifepristone, minaprine, minocycline, misoprostol, molsidomine, moroxydine, moxalactam, mupirocin, nefopam, nicardipine, nimesulide, norharman, oxytocin, paroxetine, perhexiline, phenformin, pimethixene, piperlongumine, pirenzepine, probenecid, procaine, propranolol, protriptyline, pyrilamine, quercetin, quinacrine, quinine, rescinnamine, risperidone, ritodrine, saquinavir, scoulerine, sulfadimethoxine, sulfaphenazole, syrosingopine, tamoxifen, terconazole, thioproperazine, thiothixene(cis), tobramycin, tolbutamide, trifluoperazine, trimetazidine, viloxazine, xylazine, acetylsalicylsalicylic acid, nimetazepam, clobazam, alimemazine, tranilast, ebastine, pranlukast, methyclothiazide, alacepril, clinofibrate, acetylcysteine, buformin, terguride, stanozolol, mestanolone, pantethine, limaprost, sarpogrelate, argatroban, fludroxycortide, sulfadoxine, ubenimex, celecoxib, 6-furfurylaminopurine, solasodine, gossypol, fluorocurarine, pempidine, nitrarine, promazine, sulfabenzamide, althiazide, α-ergocryptine, ebselen, furaltadone, pyrithyldione, benzthiazide, levobunolol, raloxifene, luteolin, valdecoxib, carboprost, gabexate, or a derivative thereof capable of binding to target protein Y (described later), or a salt thereof.

Regulator I, in addition to a substance that regulates the expression or function of target gene Y, can comprise any carrier, for example, a pharmaceutically acceptable carrier.

Examples of the pharmaceutically acceptable carrier include, but are not limited to, excipients such as sucrose, starch, marmite, sorbit, lactose, glucose, cellulose, talc, calcium phosphate, and calcium carbonate; binders such as cellulose, methylcellulose, hydroxypropylcellulose, polypropylpyrrolidone, gelatin, gum arabic, polyethylene glycol, sucrose, and starch; disintegrants such as starch, carboxymethylcellulose, hydroxypropylstarch, sodium-glycol-starch, sodium hydrogen carbonate, calcium phosphate, and calcium citrate; lubricants such as magnesium stearate, Aerosil, talc, and sodium lauryl sulfate; flavoring agents such as citric acid, menthol, glycyrrhizin ammonium salt, glycine, and orange powder; preservatives such as sodium benzoate, sodium hydrogen sulfite, methyl paraben, and propyl paraben; stabilizers such as citric acid, sodium citrate, and acetic acid; suspending agents such as methylcellulose, polyvinylpyrrolidone, and aluminum stearate; dispersing agents such as surfactants; diluents such as water, physiological saline, and orange juice; base waxes such as cacao fat, polyethylene glycol, and kerosene, and the like.

Preparations suitable for oral administration include liquids comprising an effective amount of substance dissolved in a diluent such as water, physiological saline, or orange juice, capsules, sachets or tablets comprising an effective amount of substance in the form of solid or granules, suspensions comprising an effective amount of substance suspended in an appropriate dispersant, emulsions comprising a solution of an effective amount of substance dispersed in an appropriate dispersant and the like.

Preparations suitable for parenteral administration (e.g., subcutaneous injection, intramuscular injection, topical injection, intraperitoneal injection, and the like) include aqueous and non-aqueous isotonic sterile injection liquids, which may comprise an antioxidant, a buffer solution, a bacteriostatic agent, an isotonizing agent and the like. Other examples are aqueous and non-aqueous sterile suspensions, which may comprise a suspending agent, a solubilizer, a thickening agent, a stabilizer, an antiseptic and the like. The preparation can be included in a container in a unit dose or multiple doses like an ampoule or vial. It is also possible to lyophilize the active ingredient and a pharmaceutically acceptable carrier and preserve them in a state that only requires dissolving or suspending in a suitable sterile vehicle immediately before use.

The dose of regulator I varies depending on the activity and kind of the active ingredient, severity of the disease, the animal species to be the administration subject, drug acceptability, body weight and age of the administration subject, and the like, it is generally about 0.001 to about 500 mg/kg a day for an adult based on the amount of the active ingredient.

Regulator I enables the regulation, for example, suppression or promotion, of an action associated with a bioactive substance X. Hence, regulator I is useful for the prophylaxis and treatment of a disease or condition associated with bioactive substance X, and as an investigational reagent for the disease or the condition, and the like.

3.2. Regulator of a Function Associated with a Target Protein Y (Regulator II)

The present invention provides a regulator of a function associated with a target protein Y, which comprises bioactive substance X.

This regulator is referred to as “regulator II” as required.

The bioactive substance X can be trimethylcolchicic acid, acenocoumarol, paracetamol, acetohexamide, acetopromazine, actinomycin D, ajmaline, albendazole, alfuzosin, α-methyl-5-hydroxytryptamine, amoxapine, antipyrine, azithromycin, benzbromarone, benzethonium, benzydamine, berberine, bezafibrate, bicartamide, boldine, bromperidol, budesonide, bupivacaine, buspirone, cefazolin, celestine blue, cephaeline, chlordiazepoxide, chlorogenic acid, chlorothiazide, chromomycin A3, ciclopirox, cisapride, clarithromycin, clemizole, clenbuterol, clobetasone, clofazimine, clofilium, clomiphene, clopamide, colchicine, colistin, conessine, coniine (DL), coralyne, cyclobenzaprine, cyclopentolate, cyclosporine A, diclofenac, dichlorphenamide, diflunisal, dihydrostreptomycin, diperodon, difenidol, dipyridamole, dizocilpine, DO897/99, domperidone, dopamine, doxazosin, doxycycline, eburnamonine, etodolac, fenbendazole, fenbufen, fenoprofen, flumequine, flupentixol, fluphenazine, fluvoxamine, furazolidone, gabapentin, GBR12909, glibenclamide, glipizide, gramicidin, guanfacine, harmol, hydroflumethiazide, hydroxychloroquine, hydroxytacrine(R,S), ifosfamide, iobenguane, iproniazid, isoxicam, isradipine, josamycin, ketoprofen, 3-hydroxykynurenine, leuprolide, L-thyroxine, lidoflazine, α-lobeline (−), loperamide, maprotiline, mebendazole, meclofenamic acid, metanephrine (D,L), metaproterenol, metergotamine, methimazole, methoxamine, methoxy-6-harmalan, mifepristone, minaprine, minocycline, misoprostol, molsidomine, moroxydine, moxalactam, mupirocin, nefopam, nicardipine, nimesulide, norharman, oxytocin, paroxetine, perhexiline, phenformin, pimethixene, piperlongumine, pirenzepine, probenecid, procaine, propranolol, protriptyline, pyrilamine, quercetin, quinacrine, quinine, rescinnamine, risperidone, ritodrine, saquinavir, scoulerine, sulfadimethoxine, sulfaphenazole, syrosingopine, tamoxifen, terconazole, thioproperazine, thiothixene(cis), tobramycin, tolbutamide, trifluoperazine, trimetazidine, viloxazine, xylazine, acetylsalicylsalicylic acid, nimetazepam, clobazam, alimemazine, tranilast, ebastine, pranlukast, methyclothiazide, alacepril, clinofibrate, acetylcysteine, buformin, terguride, stanozolol, mestanolone, pantethine, limaprost, sarpogrelate, argatroban, fludroxycortide, sulfadoxine, ubenimex, celecoxib, 6-furfurylaminopurine, solasodine, gossypol, fluorocurarine, pempidine, nitrarine, promazine, sulfabenzamide, althiazide, α-ergocryptine, ebselen, furaltadone, pyrithyldione, benzthiazide, levobunolol, raloxifene, luteolin, valdecoxib, carboprost, gabexate, or a derivative thereof capable of binding to target protein Y (described later), or a salt thereof.

Regulator II can comprise, in addition to bioactive substance X, any carrier, for example, a pharmaceutically acceptable carrier. The dose of regulator II is the same as that of regulator I.

Regulator II enables the regulation, for example, suppression or promotion, of a function associated with a target protein Y. Hence, regulator II is useful for the prophylaxis and treatment of a disease or condition associated with target gene Y, and as an investigational reagent for the disease, and the like.

### 4. Derivative Production Method and Product Obtained by the Method

### 4.1. Derivative Production Method

The present invention provides a method of producing a bioactive substance derivative, which comprises derivatizing a bioactive substance so as to be able to regulate the expression or function of the target gene.

Derivatization means that a compound obtained by replacing a particular atom or group in a lead compound with another atom or group, or a compound obtained by subjecting a lead compound to an addition reaction, is virtually or actually synthesized. For example, the lead compound can be bioactive substance X.

The derivatization of bioactive substance X can be performed so that the regulatory capability for the expression or function of target gene Y is retained, and as required, in view of other properties of the derivative obtained, such as hydrophilicity/liphophilicity, stability, dynamics, bioavailability, toxicity and the like. The derivatization of bioactive substance X can be performed so that, for example, the regulatory capability for the expression or function of target gene Y can be increased. The derivatization of bioactive substance X can also be performed so that a function associated with a target protein Y can be regulated.

The derivatization of bioactive substance X such that the regulatory capability for the expression or function of target gene Y is retained can be performed on the basis of, for example, SBDD (structure-based drug design: SBDD) and CADD (computer-aided drug design). Examples of the design include virtual screening, de novo design, pharmacophore analysis, QSAR (quantitative structure activity relationship) and the like. If information on the steric structure of the protein itself or the target site of the protein is required during such designing, information on the steric structure is used provided that the steric structure is known by a structural analytical technique such as NMR, X-ray crystallographic analysis, or synchrotron radiation analysis. If the steric structure is unknown, information obtained by a structural predictive method such as the homology method or the threading method is used. In virtual screening, a program known per se is used; examples of the program include DOCK (Kuntz, I. D. et al., Science, 1992, 257, 1078), Gold (Jones, G. et al., J. Mol. Biol., 1995, 245, 43), FlexX (Rarey, M. et al., J. Mol. Biol., 1996, 261, 470), AtutoDock (Morris, G. M. et al., J. Comput. Chem., 1998, 19, 1639), ICM (Abagyan, R. A. et al., J. Comput. Chem., 1994, 15, 488) and the like.

The derivatization of bioactive substance X such that the regulatory capacity for the expression or function of target gene Y is retained can also be performed on the basis of, for example, biological verification (in vitro or in vivo method). In this case, for example, the above-described methodologies I to IV can be used. Furthermore, one of the above-described m methods such as SBDD and CADD, and biological verification may be used in combination.

The particular atom in bioactive substance X (a lead compound), which is substituted for producing the derivative, may be any atom present in the lead compound, exemplified by a hydrogen atom, a halogen atom (e.g., fluorine atom, chlorine atom, bromine atom, iodine atom), an oxygen atom, a sulfur atom, a nitrogen atom, a carbon atom and the like.

The particular group in bioactive substance X, which is substituted for producing the derivative, may be any group present in bioactive substance X, and can, for example, be a group having a molecular weight of 1 to 500, preferably 1 to 300, more preferably 1 to 200, most preferably 1 to 100. Examples of the particular group include an optionally substituted C₁ to C₈ hydrocarbon group, an optionally substituted C₁ to C₈ acyl group, an optionally substituted aromatic or non-aromatic C₃ to C₁₄ hydrocarbon cyclic group, or an optionally substituted aromatic or non-aromatic C₃ to C₁₄ heterocyclic group, an amino group, an amino group mono- or di-substituted by an alkyl group having 1 to 4 carbon atoms or an acyl group having 2 to 8 carbon atoms, an amidino group, a carbamoyl group, a carbamoyl group mono- or di-substituted by an alkyl group having 1 to 4 carbon atoms, a sulfamoyl group, a sulfamoyl group mono- or di-substituted by an alkyl group having 1 to 4 carbon atoms, a carboxyl group, an alkoxycarbonyl group having 2 to 8 carbon atoms, a hydroxy group, an alkoxy group having 1 to 6 carbon atoms optionally substituted by 1 to 3 halogen atoms, an alkenyloxy group having 2 to 5 carbon atoms optionally substituted by 1 to 3 halogen atoms, a cycloalkyloxy group having 3 to 7 carbon atoms, an aralkyloxy group having 7 to 9 carbon atoms, an aryloxy group having 6 to 14 carbon atoms, a thiol group, an alkylthio group having 1 to 6 carbon atoms optionally substituted by 1 to 3 halogen atoms, an aralkylthio group having 7 to 9 carbon atoms, an arylthio group having 6 to 14 carbon atoms, a sulfo group, a cyano group, an azido group, a nitro group, a nitroso group and the like.

The optionally substituted C₁ to C₈ hydrocarbon group can, for example, be an optionally substituted C₁ to C₈ alkyl group, an optionally substituted C₂ to C₈ alkenyl group, or an optionally substituted C₂ to C₈ alkinyl group.

The C₁ to C₈ alkyl group in the optionally substituted C₁ to C₈ alkyl group may be linear or branched, preferably having 1 to 6 carbon atoms; examples include methyl, ethyl, propyl, isopropyl, butyl, isobutyl, sec-butyl, tert-butyl and the like.

The C₂ to C₈ alkenyl group in the optionally substituted C₂ to C₈ alkenyl group may be linear or branched, preferably having 2 to 6 carbon atoms; examples include ethenyl, 1-propenyl, 2-propenyl, 2-methyl-1-propenyl, 1-butenyl, 2-butenyl, 3-butenyl and the like.

The C₂ to C₈ alkinyl group in the optionally substituted C₂ to C8 alkinyl group may be linear or branched, preferably having 2 to 6 carbon atoms; examples include ethynyl, 1-propynyl, 2-propynyl, 1-buthynyl, 2-buthynyl, 3-buthynyl and the like.

The C₁ to C₈ acyl group in the optionally substituted C₁ to C₈ acyl group may be linear or branched, preferably having 2 to 6 carbon atoms; examples include formyl, acetyl, propinoyl, butanoyl, 2-methylpropinoyl and the like.

The aromatic C₃ to C₁₄ hydrocarbon cyclic group in the optionally substituted aromatic C₃ to C₁₄ hydrocarbon cyclic group may be monocyclic, bicyclic or tricyclic, preferably having 3 to 12 carbon atoms; examples include phenyl and naphthyl.

The non-aromatic C₃ to C₁₄ hydrocarbon cyclic group in the optionally substituted non-aromatic C₃ to C₁₄ hydrocarbon cyclic group may be saturated or unsaturated monocyclic, bicyclic or tricyclic, preferably having 3 to 12 carbon atoms; examples include cycloalkyl groups (e.g., cyclopropyl, cyclobutyl, cyclopentyl, cyclohexyl, cycloheptyl, cyclooctyl), cycloalkenyl groups (e.g., 2-cyclopenten-1-yl, 3-cyclopenten-1-yl, 2-cyclohexen-1-yl, 3-cyclohexen-1-yl), cycloalkadienyl groups (e.g., 2,4-cyclopentadien-1-yl, 2,4-cyclohexadien-1-yl, 2,5-cyclohexadien-1-yl) and the like.

The aromatic C₃ to C₁₄ heterocyclic group in the optionally substituted aromatic C₃ to C₁₄ heterocyclic group is a monocyclic, bicyclic or tricyclic aromatic heterocyclic group containing 1 to 5 hetero atoms selected from among oxygen atoms, sulfur atoms and nitrogen atoms, in addition to carbon atoms, as the ring-forming atoms, preferably having 3 to 12 carbon atoms. Examples of the monocyclic aromatic C₃ to C₁₄ heterocyclic group include furyl, thienyl, pyrrolyl, oxazolyl, isooxazolyl, thiazolyl, isothiazolyl, imidazolyl, pyrazolyl, oxadiazolyl, furazanyl, thiadiazolyl, triazolyl, tetrazolyl, pyridyl, pyrimidinyl, pyridazinyl, pyrazinyl, triazinyl and the like. Examples of the bicyclic or tricyclic aromatic heterocyclic group include benzofuranyl, isobenzofuranyl, benzo[b]thienyl, indolyl, isoindolyl, 1H-indazolyl, benzimidazolyl, benzooxazolyl, benzothiazolyl, 1H-benzotriazolyl, quinolyl, isoquinolyl, cinnolyl, quinazolyl, quinoxalinyl, phthaladinyl, naphthylizinyl, purinyl, pteridinyl, carbazolyl, α-carbonylyl, β-carbonylyl, γ-carbonylyl, acrydinyl, phenoxyzinyl, phenothiazinyl, phenadinyl, phenoxathiinyl, thianthrenyl, indolidinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl and the like.

The non-aromatic C₃ to C₁₄ heterocyclic group in the optionally substituted non-aromatic C₃ to C₁₄ heterocyclic group is a monocyclic, bicyclic or tricyclic saturated or unsaturated heterocyclic group containing 1 to 5 hetero atoms selected from among oxygen atoms, sulfur atoms and nitrogen atoms, in addition to carbon atoms, as the ring-forming atoms, preferably having 3 to 12 carbon atoms; examples include oxiranyl, azetidinyl, oxetanyl, thietanyl, pyrrolidinyl, tetrahydrofuryl, tetrahydropyranyl, morpholinyl, thiomorpholinyl, piperazinyl, pyrrolidinyl, piperidino, morpholino, thiomorpholino and the like.

The kind of the substituent in any group optionally substituted can be the same as the particular group in bioactive substance X (described above), which is substituted for producing the derivative.

The number of particular atoms or groups in bioactive substance X, which is substituted for producing the derivative is any one, as long as the derivative produced is capable of regulating the expression or function of the gene Y, for example, as long as it is capable of binding to target protein Y, and can be, for example, 1 to 10, preferably 1 to 5, more preferably 1 to 3, further more preferably 1 to 2, most preferably 1.

The kind of a particular atom or group used for substitution (i.e., an atom or group introduced to the substitution site) can be the same as the particular atom or group in bioactive substance X, which is substituted for producing the derivative.

The atom or group added to bioactive substance X for producing the derivative (i.e., an atom or group used in the addition reaction) is an atom permitting an addition reaction, for example, an atom such as the hydrogen atom or the halogen atom, or a group capable of acting as a nucleophile or electrophile, out of the particular atoms or groups in bioactive substance X (described above), which is substituted for producing the derivative.

The number of atoms or groups added to bioactive substance X for producing the derivative is any one, as long as the derivative produced is capable of regulating the expression or function of the gene Y, for example, as long as it is capable of binding to target protein Y, and can be, for example, less than 6, preferably less than 4, more preferably less than 2.

The production method of the present invention is useful for, for example, the development of prophylactic or therapeutic agents for diseases or conditions associated with bioactive substance X or diseases or conditions associated with target gene Y, or investigational reagents for the diseases or the conditions, and the like.

### 4.2. Products Obtained by the Derivative Production Method

The present invention provides a product obtained by the above-described method of producing a derivative.

The product provided by the above-described production method can be a bioactive substance X derivative obtained by the production method of the present invention, and a bioactivity regulator comprising the derivative (described above).

A product provided by the above-described production method is useful for, for example, the prophylaxis or treatment of a disease or condition associated with bioactive substance X, or a disease or condition associated with target gene Y, or as investigational reagents for the disease or the condition, and the like.

### 5. Complex and a Method of Producing the Same

The present invention provides a complex comprising a bioactive substance and a target protein therefor.

The bioactive substance can be, for example, the above-mentioned bioactive substance X. In detail, the bioactive substance X can be trimethylcolchicic acid, acenocoumarol, paracetamol, acetohexamide, acetopromazine, actinomycin D, ajmaline, albendazole, alfuzosin, α-methyl-5-hydroxytryptamine, amoxapine, antipyrine, azithromycin, benzbromarone, benzethonium, benzydamine, berberine, bezafibrate, bicartamide, boldine, bromperidol, budesonide, bupivacaine, buspirone, cefazolin, celestine blue, cephaeline, chlordiazepoxide, chlorogenic acid, chlorothiazide, chromomycin A3, ciclopirox, cisapride, clarithromycin, clemizole, clenbuterol, clobetasone, clofazimine, clofilium, clomiphene, clopamide, colchicine, colistin, conessine, coniine (DL), coralyne, cyclobenzaprine, cyclopentolate, cyclosporine A, diclofenac, dichlorphenamide, diflunisal, dihydrostreptomycin, diperodon, difenidol, dipyridamole, dizocilpine, DO897/99, domperidone, dopamine, doxazosin, doxycycline, eburnamonine, etodolac, fenbendazole, fenbufen, fenoprofen, flumequine, flupentixol, fluphenazine, fluvoxamine, furazolidone, gabapentin, GBR12909, glibenclamide, glipizide, gramicidin, guanfacine, harmol, hydroflumethiazide, hydroxychloroquine, hydroxytacrine(R,S), ifosfamide, iobenguane, iproniazid, isoxicam, isradipine, josamycin, ketoprofen, 3-hydroxykynurenine, leuprolide, L-thyroxine, lidoflazine, α-lobeline (−), loperamide, maprotiline, mebendazole, meclofenamic acid, metanephrine (D,L), metaproterenol, metergotamine, methimazole, methoxamine, methoxy-6-harmalan, mifepristone, minaprine, minocycline, misoprostol, molsidomine, moroxydine, moxalactam, mupirocin, nefopam, nicardipine, nimesulide, norharman, oxytocin, paroxetine, perhexiline, phenformin, pimethixene, piperlongumine, pirenzepine, probenecid, procaine, propranolol, protriptyline, pyrilamine, quercetin, quinacrine, quinine, rescinnamine, risperidone, ritodrine, saquinavir, scoulerine, sulfadimethoxine, sulfaphenazole, syrosingopine, tamoxifen, terconazole, thioproperazine, thiothixene(cis), tobramycin, tolbutamide, trifluoperazine, trimetazidine, viloxazine, xylazine, acetylsalicylsalicylic acid, nimetazepam, clobazam, alimemazine, tranilast, ebastine, pranlukast, methyclothiazide, alacepril, clinofibrate, acetylcysteine, buformin, terguride, stanozolol, mestanolone, pantethine, limaprost, sarpogrelate, argatroban, fludroxycortide, sulfadoxine, ubenimex, celecoxib, 6-furfurylaminopurine, solasodine, gossypol, fluorocurarine, pempidine, nitrarine, promazine, sulfabenzamide, althiazide, α-ergocryptine, ebselen, furaltadone, pyrithyldione, benzthiazide, levobunolol, raloxifene, luteolin, valdecoxib, carboprost, gabexate, or a derivative thereof capable of binding to target protein Y. The kind of bioactive substance X can be selected as appropriate according to the kind of target protein Y.

The target protein for the bioactive substance can be, for example, the above-described target protein Y. Specifically, target protein Y can be a protein comprising the amino acid sequence shown by SEQ ID NOs:1 to 63 or a protein homologous thereto or a variant thereof. The kind of target protein Y used to form the complex can be selected as appropriate according to the kind of bioactive substance X.

As one embodiment, the complex of the present invention can be trimethylcolchicic acid, acenocoumarol, paracetamol, acetohexamide, acetopromazine, actinomycin D, ajmaline, albendazole, alfuzosin, α-methyl-5-hydroxytryptamine, amoxapine, antipyrine, azithromycin, benzbromarone, benzethonium, benzydamine, berberine, bezafibrate, bicartamide, boldine, bromperidol, budesonide, bupivacaine, buspirone, cefazolin, celestine blue, cephaeline, chlordiazepoxide, chlorogenic acid, chlorothiazide, chromomycin A3, ciclopirox, cisapride, clarithromycin, clemizole, clenbuterol, clobetasone, clofazimine, clofilium, clomiphene, clopamide, colchicine, colistin, conessine, coniine (DL), coralyne, cyclobenzaprine, cyclopentolate, cyclosporine A, diclofenac, dichlorphenamide, diflunisal, dihydrostreptomycin, diperodon, difenidol, dipyridamole, dizocilpine, DO897/99, domperidone, dopamine, doxazosin, doxycycline, eburnamonine, etodolac, fenbendazole, fenbufen, fenoprofen, flumequine, flupentixol, fluphenazine, fluvoxamine, furazolidone, gabapentin, GBR12909, glibenclamide, glipizide, gramicidin, guanfacine, harmol, hydroflumethiazide, hydroxychloroquine, hydroxytacrine(R,S), ifosfamide, iobenguane, iproniazid, isoxicam, isradipine, josamycin, ketoprofen, 3-hydroxykynurenine, leuprolide, L-thyroxine, lidoflazine, α-lobeline (−), loperamide, maprotiline, mebendazole, meclofenamic acid, metanephrine (D,L), metaproterenol, metergotamine, methimazole, methoxamine, methoxy-6-harmalan, mifepristone, minaprine, minocycline, misoprostol, molsidomine, moroxydine, moxalactam, mupirocin, nefopam, nicardipine, nimesulide, norharman, oxytocin, paroxetine, perhexiline, phenformin, pimethixene, piperlongumine, pirenzepine, probenecid, procaine, propranolol, protriptyline, pyrilamine, quercetin, quinacrine, quinine, rescinnamine, risperidone, ritodrine, saquinavir, scoulerine, sulfadimethoxine, sulfaphenazole, syrosingopine, tamoxifen, terconazole, thioproperazine, thiothixene(cis), tobramycin, tolbutamide, trifluoperazine, trimetazidine, viloxazine, xylazine, acetylsalicylsalicylic acid, nimetazepam, clobazam, alimemazine, tranilast, ebastine, pranlukast, methyclothiazide, alacepril, clinofibrate, acetylcysteine, buformin, terguride, stanozolol, mestanolone, pantethine, limaprost, sarpogrelate, argatroban, fludroxycortide, sulfadoxine, ubenimex, celecoxib, 6-furfurylaminopurine, solasodine, gossypol, fluorocurarine, pempidine, nitrarine, promazine, sulfabenzamide, althiazide, α-ergocryptine, ebselen, furaltadone, pyrithyldione, benzthiazide, levobunolol, raloxifene, luteolin, valdecoxib, carboprost, gabexate, or a derivative thereof capable of binding to a target protein and a complex according to a combination of the target protein therefor.

In another embodiment, the complex of the present invention can be a complex according to a combination of a protein comprising the amino acid sequence shown by SEQ ID NOs:1 to 63 or a protein homologous thereto or a variant thereof and a bioactive substance capable of binding to the protein.

The complex of the present invention can be preferably a complex according to any combination of (a1) to (a192) above or (b1) to (b63) above, and more preferably a complex according to any combination of (c1) to (c192) below:
(c1) a combination of trimethylcolchicic acid and a protein comprising the amino acid sequence shown by SEQ ID NO:1 or SEQ ID NO:2;
(c2) a combination of acenocoumarol and a protein comprising the amino acid sequence shown by SEQ ID NO:27;
(c3) a combination of paracetamol and a protein comprising the amino acid sequence shown by SEQ ID NO:3;
(c4) a combination of acetohexamide and a protein comprising the amino acid sequence shown by SEQ ID NO:23, SEQ ID NO:24 or SEQ ID NO:34;
(c5) a combination of acetopromazine and a protein comprising the amino acid sequence shown by SEQ ID NO:36;
(c6) a combination of actinomycin D and a protein comprising the amino acid sequence shown by SEQ ID NO:54;
(c7) a combination of ajmaline and a protein comprising the amino acid sequence shown by SEQ ID NO:1 or SEQ ID NO:2;
(c8) a combination of albendazole and a protein comprising the amino acid sequence shown by SEQ ID NO:38;
(c9) a combination of alfuzosin and a protein comprising the amino acid sequence shown by SEQ ID NO:35;
(c10) a combination of α-methyl-5-hydroxytryptamine and a protein comprising the amino acid sequence shown by SEQ ID NO:30;
(c11) a combination of amoxapine and a protein comprising the amino acid sequence shown by SEQ ID NO:36;
(c12) a combination of antipyrine and a protein comprising the amino acid sequence shown by SEQ ID NO:1;
(c13) a combination of azithromycin and a protein comprising the amino acid sequence shown by SEQ ID NO:62;
(c14) a combination of benzbromarone and a protein comprising the amino acid sequence shown by SEQ ID NO:42, SEQ ID NO:53 or SEQ ID NO:54;
(c15) a combination of benzethonium and a protein comprising the amino acid sequence shown by SEQ ID NO:23, SEQ ID NO:53 or SEQ ID NO:61;
(c16) a combination of benzydamine and a protein comprising the amino acid sequence shown by SEQ ID NO:60;
(c17) a combination of berberine and a protein comprising the amino acid sequence shown by SEQ ID NO:32;
(c18) a combination of bezafibrate and a protein comprising the amino acid sequence shown by SEQ ID NO:39;
(c19) a combination of bicartamide and a protein comprising the amino acid sequence shown by SEQ ID NO:53;
(c20) a combination of boldine and a protein comprising the amino acid sequence shown by SEQ ID NO:1;
(c21) a combination of bromperidol and a protein comprising the amino acid sequence shown by SEQ ID NO:33;
(c22) a combination of budesonide and a protein comprising the amino acid sequence shown by SEQ ID NO:27;
(c23) a combination of bupivacaine and a protein comprising the amino acid sequence shown by SEQ ID NO:14;
(c24) a combination of buspirone and a protein comprising the amino acid sequence shown by SEQ ID NO:29;
(c25) a combination of cefazolin and a protein comprising the amino acid sequence shown by SEQ ID NO:59;
(c26) a combination of celestine blue and a protein comprising the amino acid sequence shown by SEQ ID NO:1, SEQ ID NO:2, SEQ ID NO:3, SEQ ID NO:32 or SEQ ID NO:46;
(c27) a combination of cephaeline and a protein comprising the amino acid sequence shown by SEQ ID NO:1 or SEQ ID NO:36;
(c28) a combination of chlordiazepoxide and a protein comprising the amino acid sequence shown by SEQ ID NO:56;
(c29) a combination of chlorogenic acid and a protein comprising the amino acid sequence shown by SEQ ID NO:27;
(c30) a combination of chlorothiazide and a protein comprising the amino acid sequence shown by SEQ ID NO:27;
(c31) a combination of chromomycin A3 and a protein comprising the amino acid sequence shown by SEQ ID NO:17 or SEQ ID NO:34;
(c32) a combination of ciclopirox and a protein comprising the amino acid sequence shown by SEQ ID NO:1 or SEQ ID NO:3;
(c33) a combination of cisapride and a protein comprising the amino acid sequence shown by SEQ ID NO:31;
(c34) a combination of clarithromycin and a protein comprising the amino acid sequence shown by SEQ ID NO:49;
(c35) a combination of clemizole and a protein comprising the amino acid sequence shown by SEQ ID NO:22 or SEQ ID NO:47;
(c36) a combination of clenbuterol and a protein comprising the amino acid sequence shown by SEQ ID NO:23, SEQ ID NO:36 or SEQ ID NO:60;
(c37) a combination of clobetasone and a protein comprising the amino acid sequence shown by SEQ ID NO:35;
(c38) a combination of clofazimine and a protein comprising the amino acid sequence shown by SEQ ID NO:15, SEQ ID NO:37, SEQ ID NO:53 or SEQ ID NO:54;
(c39) a combination of clofilium and a protein comprising the amino acid sequence shown by SEQ ID NO:1;
(c40) a combination of clomiphene and a protein comprising the amino acid sequence shown by SEQ ID NO:23;
(c41) a combination of clopamide and a protein comprising the amino acid sequence shown by SEQ ID NO:23;
(c42) a combination of colchicine and a protein comprising the amino acid sequence shown by SEQ ID NO:59;
(c43) a combination of colistin and a protein comprising the amino acid sequence shown by SEQ ID NO:62;
(c44) a combination of conessine and a protein comprising the amino acid sequence shown by SEQ ID NO:1;
(c45) a combination of coniine (DL) and a protein comprising the amino acid sequence shown by SEQ ID NO:1 or SEQ ID NO:3;
(c46) a combination of coralyne and a protein comprising the amino acid sequence shown by SEQ ID NO:33;
(c47) a combination of cyclobenzaprine and a protein comprising the amino acid sequence shown by SEQ ID NO:23;
(c48) a combination of cyclopentolate and a protein comprising the amino acid sequence shown by SEQ ID NO:36;
(c49) a combination of cyclosporine A and a protein comprising the amino acid sequence shown by SEQ ID NO:50;
(c50) a combination of diclofenac and a protein comprising the amino acid sequence shown by SEQ ID NO:27;
(c51) a combination of dichlorphenamide and a protein comprising the amino acid sequence shown by SEQ ID NO:51;
(c52) a combination of diflunisal and a protein comprising the amino acid sequence shown by SEQ ID NO:32;
(c53) a combination of dihydrostreptomycin and a protein comprising the amino acid sequence shown by SEQ ID NO:19;
(c54) a combination of diperodon and a protein comprising the amino acid sequence shown by SEQ ID NO:27;
(c55) a combination of difenidol and a protein comprising the amino acid sequence shown by SEQ ID NO:1;
(c56) a combination of dipyridamole and a protein comprising the amino acid sequence shown by SEQ ID NO:15;
(c57) a combination of dizocilpine and a protein comprising the amino acid sequence shown by SEQ ID NO:25;
(c58) a combination of DO897/99 and a protein comprising the amino acid sequence shown by SEQ ID NO:27 or SEQ ID NO:34;
(c59) a combination of domperidone and a protein comprising the amino acid sequence shown by SEQ ID NO:34, SEQ ID NO:36, SEQ ID NO:53 or SEQ ID NO:54;
(c60) a combination of dopamine and a protein comprising the amino acid sequence shown by SEQ ID NO:30;
(c61) a combination of doxazosin and a protein comprising the amino acid sequence shown by SEQ ID NO:1, SEQ ID NO:35, SEQ ID NO:53 or SEQ ID NO:61;
(c62) a combination of doxycycline and a protein comprising the amino acid sequence shown by SEQ ID NO:59;
(c63) a combination of eburnamonine and a protein comprising the amino acid sequence shown by SEQ ID NO:10 or SEQ ID NO:44;
(c64) a combination of etodolac and a protein comprising the amino acid sequence shown by SEQ ID NO:23;
(c65) a combination of fenbendazole and a protein comprising the amino acid sequence shown by SEQ ID NO:22;
(c66) a combination of fenbufen and a protein comprising the amino acid sequence shown by SEQ ID NO:59;
(c67) a combination of fenoprofen and a protein comprising the amino acid sequence shown by SEQ ID NO:26;
(c68) a combination of flumequine and a protein comprising the amino acid sequence shown by SEQ ID NO:56;
(c69) a combination of flupentixol and a protein comprising the amino acid sequence shown by SEQ ID NO:23 or SEQ ID NO:34;
(c70) a combination of fluphenazine and a protein comprising the amino acid sequence shown by SEQ ID NO:34 or SEQ ID NO:61;
(c71) a combination of fluvoxamine and a protein comprising the amino acid sequence shown by SEQ ID NO:25;
(c72) a combination of furazolidone and a protein comprising the amino acid sequence shown by SEQ ID NO:52;
(c73) a combination of gabapentin and a protein comprising the amino acid sequence shown by SEQ ID NO:59;
(c74) a combination of GBR12909 and a protein comprising the amino acid sequence shown by SEQ ID NO:61;
(c75) a combination of glibenclamide and a protein comprising the amino acid sequence shown by SEQ ID NO:23 or SEQ ID NO:37;
(c76) a combination of glipizide and a protein comprising the amino acid sequence shown by SEQ ID NO:23;
(c77) a combination of gramicidin and a protein comprising the amino acid sequence shown by SEQ ID NO:53;
(c78) a combination of guanfacine and a protein comprising the amino acid sequence shown by SEQ ID NO:23;
(c79) a combination of harmol and a protein comprising the amino acid sequence shown by SEQ ID NO:22;
(c80) a combination of hydroflumethiazide and a protein comprising the amino acid sequence shown by SEQ ID NO:11;
(c81) a combination of hydroxychloroquine and a protein comprising the amino acid sequence shown by SEQ ID NO:52;
(c82) a combination of hydroxytacrine(R,S) and a protein comprising the amino acid sequence shown by SEQ ID NO:43;
(c83) a combination of ifosfamide and a protein comprising the amino acid sequence shown by SEQ ID NO:22;
(c84) a combination of iobenguane and a protein comprising the amino acid sequence shown by SEQ ID NO:9;
(c85) a combination of iproniazid and a protein comprising the amino acid sequence shown by SEQ ID NO:19;
(c86) a combination of isoxicam and a protein comprising the amino acid sequence shown by SEQ ID NO:23;
(c87) a combination of isradipine and a protein comprising the amino acid sequence shown by SEQ ID NO:24;
(c88) a combination of josamycin and a protein comprising the amino acid sequence shown by SEQ ID NO:49;
(c89) a combination of ketoprofen and a protein comprising the amino acid sequence shown by SEQ ID NO:59;
(c90) a combination of 3-hydroxykynurenine and a protein comprising the amino acid sequence shown by SEQ ID NO:25;
(c91) a combination of leuprolide and a protein comprising the amino acid sequence shown by SEQ ID NO:50;
(c92) a combination of L-thyroxine and a protein comprising the amino acid sequence shown by SEQ ID NO:34;
(c93) a combination of lidoflazine and a protein comprising the amino acid sequence shown by SEQ ID NO:59;
(c94) a combination of α-lobeline (−) and a protein comprising the amino acid sequence shown by SEQ ID NO:6;
(c95) a combination of loperamide and a protein comprising the amino acid sequence shown by SEQ ID NO:15 or SEQ ID NO:54;
(c96) a combination of maprotiline and a protein comprising the amino acid sequence shown by SEQ ID NO:23 or SEQ ID NO:63;
(c97) a combination of mebendazole and a protein comprising the amino acid sequence shown by SEQ ID NO:32;
(c98) a combination of meclofenamic acid and a protein comprising the amino acid sequence shown by SEQ ID NO:17;
(c99) a combination of metanephrine (D,L) a protein comprising the amino acid sequence shown by SEQ ID NO:52;
(c100) a combination of metaproterenol and a protein comprising the amino acid sequence shown by SEQ ID NO:43;
(c101) a combination of metergotamine and a protein comprising the amino acid sequence shown by SEQ ID NO:25 or SEQ ID NO:43;
(c102) a combination of methimazole and a protein comprising the amino acid sequence shown by SEQ ID NO:12;
(c103) a combination of methoxamine and a protein comprising the amino acid sequence shown by SEQ ID NO:25;
(c104) a combination of methoxy-6-harmalan and a protein comprising the amino acid sequence shown by SEQ ID NO:1 or SEQ ID NO:2;
(c105) a combination of mifepristone and a protein comprising the amino acid sequence shown by SEQ ID NO:42;
(c106) a combination of minaprine and a protein comprising the amino acid sequence shown by SEQ ID NO:2;
(c107) a combination of minocycline and a protein comprising the amino acid sequence shown by SEQ ID NO:36;
(c108) a combination of misoprostol and a protein comprising the amino acid sequence shown by SEQ ID NO:23;
(c109) a combination of molsidomine and a protein comprising the amino acid sequence shown by SEQ ID NO:4;
(c110) a combination of moroxydine and a protein comprising the amino acid sequence shown by SEQ ID NO:7;
(c111) a combination of moxalactam and a protein comprising the amino acid sequence shown by SEQ ID NO:36;
(c112) a combination of mupirocin and a protein comprising the amino acid sequence shown by SEQ ID NO:24;
(c113) a combination of nefopam and a protein comprising the amino acid sequence shown by SEQ ID NO:19;
(c114) a combination of nicardipine and a protein comprising the amino acid sequence shown by SEQ ID NO:54;
(c115) a combination of nimesulide and a protein comprising the amino acid sequence shown by SEQ ID NO:27;
(c116) a combination of norharman and a protein comprising the amino acid sequence shown by SEQ ID NO:45;
(c117) a combination of oxytocin and a protein comprising the amino acid sequence shown by SEQ ID NO:49;
(c118) a combination of paroxetine and a protein comprising the amino acid sequence shown by SEQ ID NO:1 or SEQ ID NO:25;
(c119) a combination of perhexiline and a protein comprising the amino acid sequence shown by SEQ ID NO:23 or SEQ ID NO:36;
(c120) a combination of phenformin and a protein comprising the amino acid sequence shown by SEQ ID NO:36;
(c121) a combination of pimethixene and a protein comprising the amino acid sequence shown by SEQ ID NO:1;
(c122) a combination of piperlongumine and a protein comprising the amino acid sequence shown by SEQ ID NO:22;
(c123) a combination of pirenzepine and a protein comprising the amino acid sequence shown by SEQ ID NO:40;
(c124) a combination of probenecid and a protein comprising the amino acid sequence shown by SEQ ID NO:23 or SEQ ID NO:59;
(c125) a combination of procaine and a protein comprising the amino acid sequence shown by SEQ ID NO:61;
(c126) a combination of propranolol and a protein comprising the amino acid sequence shown by SEQ ID NO:22;
(c127) a combination of protriptyline and a protein comprising the amino acid sequence shown by SEQ ID NO:63;
(c128) a combination of pyrilamine and a protein comprising the amino acid sequence shown by SEQ ID NO:36 or SEQ ID NO:45;
(c129) a combination of quercetin and a protein comprising the amino acid sequence shown by SEQ ID NO:20 or SEQ ID NO:54;
(c130) a combination of quinacrine and a protein comprising the amino acid sequence shown by SEQ ID NO:61;
(c131) a combination of quinine and a protein comprising the amino acid sequence shown by SEQ ID NO:1 or SEQ ID NO:10;
(c132) a combination of rescinnamine and a protein comprising the amino acid sequence shown by SEQ ID NO:41 or SEQ ID NO:53;
(c133) a combination of risperidone and a protein comprising the amino acid sequence shown by SEQ ID NO:13 or SEQ ID NO:35;
(c134) a combination of ritodrine and a protein comprising the amino acid sequence shown by SEQ ID NO:1 or SEQ ID NO:2;
(c135) a combination of saquinavir and a protein comprising the amino acid sequence shown by SEQ ID NO:17 or SEQ ID NO:53;
(c136) a combination of scoulerine and a protein comprising the amino acid sequence shown by SEQ ID NO:2;
(c137) a combination of sulfadimethoxine and a protein comprising the amino acid sequence shown by SEQ ID NO:1;
(c138) a combination of sulfaphenazole and a protein comprising the amino acid sequence shown by SEQ ID NO:23;
(c139) a combination of syrosingopine and a protein comprising the amino acid sequence shown by SEQ ID NO:53;
(c140) a combination of tamoxifen and a protein comprising the amino acid sequence shown by SEQ ID NO:3;
(c141) a combination of terconazole and a protein comprising the amino acid sequence shown by SEQ ID NO:36;
(c142) a combination of thioproperazine and a protein comprising the amino acid sequence shown by SEQ ID NO:23 or SEQ ID NO:27;
(c143) a combination of thiothixene(cis) and a protein comprising the amino acid sequence shown by SEQ ID NO:23;
(c144) a combination of tobramycin and a protein comprising the amino acid sequence shown by SEQ ID NO:36;
(c145) a combination of tolbutamide and a protein comprising the amino acid sequence shown by SEQ ID NO:23;
(c146) a combination of trifluoperazine and a protein comprising the amino acid sequence shown by SEQ ID NO:34;
(c147) a combination of trimetazidine and a protein comprising the amino acid sequence shown by SEQ ID NO:5;
(c148) a combination of viloxazine and a protein comprising the amino acid sequence shown by SEQ ID NO:58;
(c149) a combination of xylazine and a protein comprising the amino acid sequence shown by SEQ ID NO:8;
(c150) a combination of acetylsalicylsalicylic acid and a protein comprising the amino acid sequence shown by SEQ ID NO:28;
(c151) a combination of nimetazepam and a protein comprising the amino acid sequence shown by SEQ ID NO:25;
(c152) a combination of clobazam and a protein comprising the amino acid sequence shown by SEQ ID NO:48;
(c153) a combination of alimemazine and a protein comprising the amino acid sequence shown by SEQ ID NO:1 or SEQ ID NO:2;
(c154) a combination of tranilast and a protein comprising the amino acid sequence shown by SEQ ID NO:32;
(c155) a combination of ebastine and a protein comprising the amino acid sequence shown by SEQ ID NO:54;
(c156) a combination of pranlukast and a protein comprising the amino acid sequence shown by SEQ ID NO:18, SEQ ID NO:20, SEQ ID NO:21, SEQ ID NO:35, SEQ ID NO:42, SEQ ID NO:53 or SEQ ID NO:54;
(c157) a combination of methyclothiazide and a protein comprising the amino acid sequence shown by SEQ ID NO:16 or SEQ ID NO:23;
(c158) a combination of alacepril and a protein comprising the amino acid sequence shown by SEQ ID NO:24;
(c159) a combination of clinofibrate and a protein comprising the amino acid sequence shown by SEQ ID NO:23 or SEQ ID NO:34;
(c160) a combination of acetylcysteine and a protein comprising the amino acid sequence shown by SEQ ID NO:2 or SEQ ID NO:3;
(c161) a combination of buformin and a protein comprising the amino acid sequence shown by SEQ ID NO:57;
(c162) a combination of terguride and a protein comprising the amino acid sequence shown by SEQ ID NO:9;
(c163) a combination of stanozolol and a protein comprising the amino acid sequence shown by SEQ ID NO:16;
(c164) a combination of mestanolone and a protein comprising the amino acid sequence shown by SEQ ID NO:42;
(c165) a combination of pantethine and a protein comprising the amino acid sequence shown by SEQ ID NO:1;
(c166) a combination of limaprost and a protein comprising the amino acid sequence shown by SEQ ID NO:24;
(c167) a combination of sarpogrelate and a protein comprising the amino acid sequence shown by SEQ ID NO:27;
(c168) a combination of argatroban and a protein comprising the amino acid sequence shown by SEQ ID NO:23;
(c169) a combination of fludroxycortide and a protein comprising the amino acid sequence shown by SEQ ID NO:25;
(c170) a combination of sulfadoxine and a protein comprising the amino acid sequence shown by SEQ ID NO:23;
(c171) a combination of ubenimex and a protein comprising the amino acid sequence shown by SEQ ID NO:23;
(c172) a combination of celecoxib and a protein comprising the amino acid sequence shown by SEQ ID NO:23;
(c173) a combination of 6-furfurylaminopurine and a protein comprising the amino acid sequence shown by SEQ ID NO:57;
(c174) a combination of solasodine and a protein comprising the amino acid sequence shown by SEQ ID NO:24;
(c175) a combination of gossypol and a protein comprising the amino acid sequence shown by SEQ ID NO:23;
(c176) a combination of fluorocurarine and a protein comprising the amino acid sequence shown by SEQ ID NO:10;
(c177) a combination of pempidine and a protein comprising the amino acid sequence shown by SEQ ID NO:57;
(c178) a combination of nitrarine and a protein comprising the amino acid sequence shown by SEQ ID NO:46 or SEQ ID NO:57;
(c179) a combination of promazine and a protein comprising the amino acid sequence shown by SEQ ID NO:18;
(c180) a combination of sulfabenzamide and a protein comprising the amino acid sequence shown by SEQ ID NO:23;
(c181) a combination of aithiazide and a protein comprising the amino acid sequence shown by SEQ ID NO:23;
(c182) a combination of α-ergocryptine and a protein comprising the amino acid sequence shown by SEQ ID NO:23 or SEQ ID NO:53;
(c183) a combination of ebselen and a protein comprising the amino acid sequence shown by SEQ ID NO:6;
(c184) a combination of furaltadone and a protein comprising the amino acid sequence shown by SEQ ID NO:10;
(c185) a combination of pyrithyldione and a protein comprising the amino acid sequence shown by SEQ ID NO:55;
(c186) a combination of benzthiazide and a protein comprising the amino acid sequence shown by SEQ ID NO:23 or SEQ ID NO:51;
(c187) a combination of levobunolol and a protein comprising the amino acid sequence shown by SEQ ID NO:44;
(c188) a combination of raloxifene and a protein comprising the amino acid sequence shown by SEQ ID NO:37;
(c189) a combination of luteolin and a protein comprising the amino acid sequence shown by SEQ ID NO:20 or SEQ ID NO:54;
(c190) a combination of valdecoxib and a protein comprising the amino acid sequence shown by SEQ ID NO:23;
(c191) a combination of carboprost and a protein comprising the amino acid sequence shown by SEQ ID NO:24 or SEQ ID NO:34;
(c192) a combination of gabexate and a protein comprising the amino acid sequence shown by SEQ ID NO:23.

The present invention also provides a method of producing a complex comprising a bioactive substance and a target protein therefor, which comprises bringing the bioactive substance and the target protein therefor into contact with each other. This contact can be performed by, for example, mixing the bioactive substance and the target protein in solution.

The complex of the present invention and the method of producing the complex can be useful in, for example, performing the screening methods of the present invention or the derivative production method of the present invention, or in cases where the complex is structurally analyzed to extensively investigate the mode of interaction between a bioactive substance and a target protein thereof, and the like.

### 6. Kit

The present invention provides a kit comprising a bioactive substance or a salt thereof.

In one embodiment, the kit of the present invention comprises the following (i) and (ii):
(i) a bioactive substance or a salt thereof;
(ii) a target protein for a bioactive substance, a nucleic acid that encodes the protein, an expression vector comprising the nucleic acid, cells enabling a measurement of the expression of a target gene for the bioactive substance, or an expression vector comprising the transcription regulatory region of a target gene for the bioactive substance and a reporter gene functionally linked to the region.

Provided that the kit of the present invention comprises a target protein for a bioactive substance, the protein is not in the form of a complex with the bioactive substance.

The bioactive substance, the target protein and target gene therefor, and the combination of bioactive substance and target protein therefor are the same as those described above (see, e.g., “5. Complex, and a method of producing the same”). The expression vector, the cells enabling a measurement of the expression of a target gene for a bioactive substance, the transcription regulatory region of the target gene for the bioactive substance, and the reporter gene functionally linked to the region, are the same as those described above (see, e.g., “2. Screening method, and product obtained by the method”).

The above-described kit of the present invention can be useful in, for example, performing the screening methods of the present invention, the derivative production method of the present invention, and the complex production method of the present invention and the like.

### 7. Determination Methods and Determination Kits for the Onset or Risk of Onset of Disease or Condition

The present invention provides determination methods and determination kits for the onset or risk of onset of a specified disease or condition. The determination methods and determination kits of the present invention can be roughly divided into determination methods and determination kits based on measurement of the expression level, and determination methods and determination kits based on measurement of the polymorphism. Furthermore, they can be classified into determination methods and determination kits for the onset or risk of onset of a disease or condition associated with bioactive substance X, and determination methods and determination kits for the onset or risk of onset of a disease or condition associated with target gene Y, from the viewpoint of the disease or condition for which a determination of the onset or risk of onset is desired. The individual determination methods and determination kits are hereinafter described in detail. As required, “the expression of target protein Y or the gene that encodes the protein” is sometimes referred to as “expression of target protein Y” or “expression of target gene Y”, and “function of a target protein Y or a gene that encodes the protein” is sometimes referred to as “function of a target protein Y” or “function of target gene Y” as required.

### 7.1. Determination Methods and Determination Kits for the Onset or Risk of Onset of Disease or Condition on the Basis of Measurement of the Expression Level of Target Gene Y

7.1.1. Determination Method for the Onset or Risk of Onset of Disease or Condition Associated with Bioactive Substance X on the Basis of Measurement of the Expression Level of Target Gene Y (Determination Method I)

The present invention provides a determination method for the onset or risk of onset of a disease or condition associated with bioactive substance X, which comprises measuring the expression level of target gene Y.

This determination method is referred to as “determination method I” as required.

In one embodiment, determination method I comprises the following steps (a) and (b):
(a) a step for measuring the expression level of target gene Y in a biological sample collected from an animal;
(b) a step for evaluating the onset or likelihood of onset of a disease or condition associated with bioactive substance X on the basis of the expression level of target gene Y.

The methodology comprising the above-described steps (a) to (b) is referred to as “methodology V” as required.

In step (a) of methodology V, the expression level of target gene Y in a biological sample collected from an animal is measured. Although the animal is not particulary limited, a mammal or a bird is preferable, with greater preference given to a mammal. Examples of the mammal include laboratory animals such as mice, rats, hamsters, guinea pigs, and rabbits, domestic animals such as swine, bovine, goat, horses, and sheep, companion animals such as dogs and cats, and primates such as monkeys, orangutans, chimpanzees, and humans. Examples of the bird include chicken, partridges, turkeys, and ostriches.

The biological sample may be any sample containing a tissue expressing target gene Y, or any sample containing secreted target protein Y. The sample containing a tissue expressing target gene Y differs according to the kind of target gene Y. The tissue expressing target gene Y can be examined using, for example, H-Inv DB. The sample containing secreted target protein Y differs according to the kind of target gene Y, and can, for example, be blood, plasma, serum, saliva, cerebrospinal fluid, tear, or urine.

In this step, a biological sample collected from an animal in advance is used; of course, this methodology V can further comprise a step for collecting a biological sample from an animal. Collection of a biological sample from an animal can be performed by a method known per se.

The expression level of target gene Y can be measured by a method known per se with a product, for example, a transcription product or translation product, of target gene Y, as the subject. For example, the expression level of a transcription product can be measured by preparing total RNA from the cells, and performing RT-PCR, Northern blotting and the like. The expression level of a translation product can also be measured by preparing an extract from the cells, and performing an immunological technique. Useful immunological techniques include radioisotope immunoassay (RIA), ELISA (Methods in Enzymol. 70: 419-439 (1980)), fluorescent antibody, and the like.

In step (b) of methodology V, aan assesssment is made whether or not the animal is suffering from a disease or condition associated with bioactive substance X on the basis of the expression level of target gene Y. Specifically, first, the measured expression level of target gene Y is compared with the expression level of target gene Y in an animal that has not contracted the disease or condition associated with bioactive substance X (e.g., a normal animal). This comparison of expression level is preferably performed on the basis of the presence or absence of a significant difference. The expression level of target gene Y in an animal that has not contracted the disease or condition associated with bioactive substance X can be determined by a method known per se.

Next, on the basis of the result of the comparison of the expression level of target gene Y, a judgement is made whether or not the animal is possibly suffering from a disease or condition associated with bioactive substance X, or is likely or unlikely to suffer from the same in the future. The combination of a disease or condition associated with bioactive substance X and target gene Y is the same as described above. It is known that in animals that have contracted a particular disease, a change in the expression of the gene associated with the disease is often observed. It is also known that prior to the onset of a particular disease, a change in the expression of the particular gene is often observed. Hence, by analyzing the expression level of target gene Y, it is possible to determine the onset or likelihood of onset of the disease or condition associated with bioactive substance X.

Determination method I enables a determination of the presence or absence of a disease or condition associated with bioactive substance X, or the likelihood of contracting the disease or condition. Hence, determination method I is useful for, for example, the easy and early detection of the disease or condition and the like.

7.1.2. Determination Kit for the Onset or Risk of Onset of Disease or Condition Associated with Bioactive Substance X on the Basis of Measurement of Expression Level of Target Gene Y (Determination Kit I)

The present invention provides a determination kit that enables the easy conduct of determination method I.

This determination kit is referred to as “determination kit I” as required.

In one embodiment, determination kit I comprises the following (i) and (ii):
(i) a means capable of measuring the expression level of target gene Y;
(ii) a medium recording the relationship between a disease or condition associated with bioactive substance X and the expression level of target gene Y.

The kit may further comprise a means capable of collecting a biological sample from an animal, or a transcription product of target gene Y or target protein Y and the like.

The means capable of measuring the expression level of target gene Y is not subject to limitation, as long as it allows a quantitation of the expression level of target gene Y; for example, such means are roughly divided into means capable of quantifying target protein Y, and means capable of quantifying a transcription product of target gene Y. The means may be labeled with a labeling substance. Provided that the means is not labeled with a labeling substance, the determination kit of the present invention may further comprise the labeling substance. The labeling substance is the same as described above.

Specifically, the means capable of quantifying target protein Y include an antibody against target protein Y (described above), bioactive substance X and the like. The antibody against target protein Y and bioactive substance X may be provided in a form immobilized on a substrate such as a plate.

Examples of the means capable of quantifying a transcription product of target gene Y include a nucleic acid probe for a transcription product of target gene Y, a primer pair capable of amplifying a transcription product of target gene Y and the like. The nucleic acid probe and primer pair may be provided along with a reagent for transcription product extraction.

The nucleic acid probe for the transcription product of target gene Y is not subject to limitation, as long as it enables a measurement of the amount of the transcription product of target gene Y. Although the probe may be any of DNA and RNA, preference is given to DNA in view of stability and the like. The probe may be single-stranded or double-stranded. Although the probe size is not subject to limitation, as long as it enables detection of the transcription product of target gene Y, the size is preferably about 15 to 1000 bp, more preferably about 50 to 500 bp. The probe may be provided in a form immobilized on a substrate like a microarray.

A primer pair enabling the amplification of target gene Y is selected so that a nucleotide fragment of detectable size is amplified. The nucleotide fragment of detectable size can have a length of, for example, about 100 bp or more, preferably about 200 bp or more, more preferably about 500 bp or more. Although the primer size is not subject to limitation, as long as target gene Y can be amplified, it can be preferably about 15 to 100 bp, more preferably about 18 to 50 bp, further more preferably about to 30 bp. Provided that the means capable of quantifying a transcription product of target gene Y is a primer pair capable of amplifying target gene Y, the determination kit can further comprise a reverse transcriptase.

The medium recording the relationship between a disease or condition associated with bioactive substance X and target gene Y can be one recording the difference in the expression level of target gene Y between an animal suffering from a disease or condition associated with bioactive substance X and a non-suffering animal. The medium can be a document or a computer-readable recording medium, for example, a flexible disk, CD, DVD, hard disk and the like. The expression level of target gene Y in an animal suffering from a disease or condition associated with bioactive substance X can be increased or decreased compared to an animal not suffering from the disease or the condition.

Any means can be used to collect a biological sample from an animal, as long as it allows the obtainment of the biological sample from the animal; examples include blood drawing instruments such as injectors, biopsy instruments such as biopsy needles and biopsy forceps, surgical instruments such as surgical knives and scissors, and the like.

The transcription product or target protein Y of target gene Y can be used as, for example, a control.

Determination kit I enables a determination of the presence or absence of a disease or condition associated with bioactive substance X, or the likelihood of contracting the disease or condition. Hence, determination kit I is useful for, for example, the easy and early detection of the disease or condition and the like.

### 7.2. Determination Methods and Determination Kits for the Risk of Onset of Disease or Condition on the Basis of Measurement of Polymorphism of Target Gene Y

7.2.1. Determination Method for the Risk of Onset of Disease or Condition Associated with Bioactive Substance X on the Basis of Measurement of Polymorphism of Target Gene Y (Determination Method II)

The present invention provides a determination method for the risk of onset of a disease or condition associated with bioactive substance X, which comprises measuring the polymorphism of target gene Y.

This determination method is referred to as “determination method II” as required.

In one embodiment, determination method II comprises the following steps (a) and (b):
(a) a step for measuring the polymorphism of target gene Y in a biological sample collected from an animal;
(b) a step for evaluating the likelihood of the onset of a disease or condition associated with bioactive substance X on the basis of the type of polymorphism.

The methodology comprising the above-described steps (a) to (b) is referred to as “methodology VI” as required.

In step (a) of methodology VI, the type of polymorphism of target gene Y in a biological sample collected from an animal is measured. The animal is the same as described above.

Although the biological sample used may be one described with respect to methodology V above, this methodology VI enables the use of any tissue containing genomic DNA such as hair, nails, skin or mucosa as the biological sample. In view of the ease of procurement, burden on the human body and the like, the biological sample is preferably a sample of hair, nails, skin, mucosa, blood, plasma, serum, saliva and the like.

In this step, a biological sample previously collected from an animal is used, but of course this methodology VI can further comprise a step for collecting a biological sample from an animal. Collection of a biological sample from an animal can be performed by a method known per se.

A polymorphism of target gene Y means a mutation found at a frequency in the nucleotide sequence of the genomic DNA comprising target gene Y in a certain population, and can be one or more DNA substitutions, deletions, or additions (e.g., SNP, haplotype) in the genomic DNA comprising target gene Y, and a repeat, inversion, translocation and the like of the genomic DNA. Polymorphisms of target gene Y are registered with known databases, for example, H-Inv DB and the like. The type of polymorphism of target gene Y used in this determination method is a mutation in a nucleotide sequence whose frequency differs between animals suffering from a disease or condition associated with bioactive substance X and non-suffering animals out of all types of polymorphism in target gene Y, and can be, for example, one that alters the expression of target gene Y or alters a function associated with a target protein Y (e.g., the ability of target protein Y to bind to bioactive substance X). Such types of polymorphism can be determined by a method known per se such as linkage analysis.

A determination of the type of polymorphism can be performed by a method known per se. For example, the RFLP (restriction fragment length polymorphism) method, the PCR-SSCP (single-stranded DNA conformation polymorphism) analysis method, the ASO (allele specific oligonucleotide) hybridization method, the direct sequencing method, the ARMS (amplification refracting mutation system) method, the denaturing gradient gel electrophoresis method, the RNaseA cleavage method, the DOL (dye-labeled oligonucleotide ligation) method, the TaqMan PCR method, the invader method, the MALDI-TOF/MS (matrix assisted laser desorption-time of flight/mass spectrometry) method, the TDI (template-directed dye-terminator incorporation) method and the like can be used.

In step (b) of methodology VI, assessment of the likelihood of contracting a disease or condition associated with bioactive substance X in an animal is made on the basis of the type of polymorphism. The combination of a disease or condition associated with bioactive substance X and target gene Y is the same as described above. It is known that animals susceptible to a particular disease often have a particular type of polymorphism in the gene associated with the disease. Hence, it is possible to determine the likelihood of the onset of a disease or condition associated with bioactive substance X by polymorphism analysis.

Determination method II enables a determination of the likelihood of contracting a disease or condition associated with bioactive substance X. Hence, determination method II is useful for the provision of an incentive for improving one's lifestyle for the purpose of preventing the disease or condition and the like.

7.2.2. Determination Kit for the Risk of Onset of Disease or Condition Associated with Bioactive Substance X on the Basis of Measurement of Polymorphism of Target Gene Y (Determination Kit II)

The present invention also provides a determination kit that enables the easy conduct of determination method II.

This determination kit is referred to as “determination kit II” as required.

In one embodiment, determination kit II comprises the following (i) and (ii):
(i) a means capable of measuring the polymorphism of target gene Y;
(ii) a medium recording the relationship between a disease or condition and target gene Y.

The kit may further comprise a means capable of collecting of a biological sample from an animal, or a nucleic acid that encodes target gene Y having a particular type of polymorphism, a nucleic acid that encodes target gene Y not having a particular type of polymorphism and the like.

The means capable of measuring the polymorphism of target gene Y is not subject to limitation, as long as it is capable of determining the polymorphism of target gene Y. The means may be labeled with a labeling substance. Provided that the means is not labeled with a labeling substance, this kit may further comprise the labeling substance. The labeling substance is the same as described above.

Specifically, the means capable of measuring the polymorphism of target gene Y can be a nucleic acid probe enabling a specific measurement of target gene Y having a particular type of polymorphism, or a primer pair capable of specifically amplifying target gene Y having a particular type of polymorphism. The nucleic acid probe and primer pair can be ones for a genomic DNA comprising target gene Y or for a transcription product of target gene Y. The nucleic acid probe and primer pair may be provided along with a transcription product or a reagent for genomic DNA extraction.

The nucleic acid probe enabling a specific measurement of target gene Y having a particular type of polymorphism is not subject to limitation, as long as target gene Y having a particular type of polymorphism can be selected. Although the probe may be any of DNA and RNA, preference is given to DNA in view of stability and the like. The probe may be any of single-stranded and double-stranded. The probe size is preferably as short as possible to enable selecting of target gene Y having a particular type of polymorphism, and can be, for example, a size of about 15 to 30 bp. The probe may be provided in a form immobilized on a substrate like a microarray. The probe enables, for example, ASO (allele specific oligonucleotide) hybridization method.

The primer pair capable of specifically amplifying target gene Y having a particular type of polymorphism is selected so that a nucleotide fragment of measurable size is amplified. Such a primer pair is designed so that, for example, a polymorphism site is present at the 3′ terminus of either primer. The nucleotide fragment of measurable size can, for example, have a length of about 100 bp or more, preferably about 200 bp or more, more preferably about 500 bp or more. The primer size is not subject to limitation, as long as target gene Y can be amplified, and can be preferably about 15 to 100 bp, more preferably about 18 to 50 bp, further more preferably about 20 to 30 bp. Provided that the means capable of measuring the polymorphism of target gene Y is a primer pair for a transcription product of target gene Y, the determination kit can further comprise a reverse transcription enzyme.

As another means capable of measuring the polymorphism of target gene Y, a restriction enzyme that recognizes a site of a particular type of polymorphism can be mentioned. This means enables polymorphism analysis by RFLP.

The medium recording the relationship between a disease or condition associated with bioactive substance X and target gene Y can be one recording the difference in the nucleotide sequence of the genomic DNA comprising target gene Y between an animal suffering from the disease or condition associated with bioactive substance X and a non-suffering animal. For example, the medium can be a document or a computer-readable recording medium such as a flexible disk, CD, DVD, and hard disk.

The means capable of collecting a biological sample from an animal is the same as described above.

A nucleic acid that encodes target gene Y having a particular type of polymorphism, and a nucleic acid that encodes target gene Y not having a particular type of polymorphism can, for example, be used as controls.

Determination kit II enables a determination of the likelihood of contracting a disease or condition associated with bioactive substance X. Hence, determination kit II is useful for the provision of an incentive for improving one's lifestyle for the purpose of preventing the disease or condition and the like.

7.2.3. Method of Determining the Risk of Onset of Disease or Condition Associated with Target Gene Y on the Basis of Measurement of Polymorphism of Target Gene Y (Determination Method III)

The present invention provides a determination method for the risk of onset of a disease or condition associated with target gene Y, which comprises measuring the polymorphism of target gene Y.

This determination method is referred to as “determination method III” as required.

In one embodiment, determination method III comprises the following steps (a) and (b):
(a) a step for measuring the type of the polymorphism of target protein Y in a biological sample collected from an animal;
(b) a step for evaluating the likelihood of the onset of a disease or condition associated with target gene Y on the basis of the type of polymorphism.

In determination method III, the type of polymorphism used to determine the risk of onset alters the ability of target protein Y to bind to bioactive substance X. Such type of polymorphism can be determined by a method known per se such as binding assay.

The methodology comprising steps (a) and (b) above in determination method III is the same as methodology VI except for the type of polymorphism of target gene Y to be measured.

Determination method III enables a determination of the likelihood of contracting a disease or condition associated with target gene Y. Hence, determination method III is useful for the provision of an incentive for improving one's lifestyle for the purpose of preventing the disease or condition and the like.

7.2.4. Determination Kit for the Risk of Onset of Disease or Condition Associated with Target Gene Y on the Basis of Measurement of Polymorphism of Target Gene Y (Determination Kit III)

The present invention also provides a determination kit that enables the easy conduct of determination method III.

This determination kit is referred to as “determination kit III” as required.

In one embodiment, determination kit III comprises the following (i) and (ii):
(i) a means capable of measuring the polymorphism of target gene Y;
(ii) a medium recording the relationship between a disease or condition associated with target gene Y and the polymorphism of target gene Y.

The kit may further comprise a means capable of collecting of a biological sample from an animal, or a nucleic acid that encodes target gene Y having a particular type of polymorphism, a nucleic acid that encodes target gene Y not having a particular type of polymorphism and the like.

In determination kit III, the type of polymorphism used to determine the risk of onset is one that alters the ability of target protein Y to bind to bioactive substance X. Such type of polymorphism can be determined by a method known per se such as binding assay.

The components of determination kit III are the same as those of determination kit II except for the type of polymorphism of target gene Y to be measured.

Determination kit III enables a determination of the likelihood of contracting a disease or condition associated with target gene Y. Hence, determination kit III is useful for the provision of an incentive for improving one's lifestyle for the purpose of preventing the disease or condition and the like.

### 8. Determination Methods and Determination Kits for Susceptibility to Bioactive Substances

The present invention provides determination methods and determination kits for susceptibility to a bioactive substance. The determination methods and determination kits of the present invention can be roughly divided into determination methods and determination kits based on measurement of expression level, and determination methods and determination kits based on measurement of polymorphism. Furthermore, they are classified into determination methods and determination kits for a disease or condition associated with bioactive substance X, and determination methods and determination kits for a disease or condition associated with target gene Y, from the viewpoint of a disease or condition for which a determination of susceptibility is desired. The individual determination methods and determination kits are hereinafter described in detail.

### 8.1. Determination Methods and Determination Kits for Susceptibility to Bioactive Substances on the Basis of Measurement of the Expression Level of Target Gene Y

8.1.1. Determination Method for Susceptibility to Bioactive Substance X in Disease or Condition Associated with Bioactive substance X on the Basis of Measurement of the Expression Level of Target Gene Y (Determination Method IV)

The present invention provides a determination method for susceptibility to bioactive substance X in a disease or condition associated with bioactive substance X, which comprises measuring the expression level of target gene Y.

This determination method is referred to as “determination method IV” as required.

In one embodiment, determination method IV comprises the following steps (a) and (b):
(a) a step for measuring the expression level of target gene Y in a biological sample collected from an animal;
(b) a step for predicting the effect of bioactive substance X on the basis of the expression level of target gene Y.

The methodology comprising the above-described steps (a) to (b) is referred to as “methodology VII” as required.

Step (a) of methodology VII is the same as step (a) of methodology V.

In step (b) of methodology VII, the possible effect of bioactive substance X on animals is evaluated on the basis of the expression level of target gene Y. Specifically, first, the measured expression level of target gene Y is checked against data on the correlation of the expression level of target gene Y and susceptibility to bioactive substance X. The correlation between the expression level of target gene Y and susceptibility to bioactive substance X can be determined by a method known per se.

Next, from the result of the comparison, susceptibility to bioactive substance X is estimated. The combination of bioactive substance X and target gene Y are the same as described above. It is considered that in animals expressing a target gene for a bioactive substance at high levels, their susceptibility to the bioactive substance is high (or low), and that in animals expressing the same at low levels, their susceptibility is low (or high). Hence, it is possible to determine the susceptibility of an animal to bioactive substance X by analyzing the expression level of target gene Y. For example, provided that bioactive substance X is a drug, the likelihood or unlikelihood of obtainment of desired effect of the drug, or the probability of onset of adverse effect of a drug, can be determined.

Determination method IV enables a determination of susceptibility to bioactive substance X. Hence, determination method IV is useful for, for example, the evaluation of an action of bioactive substance X on a particular animal, and the like.

8.1.2. Determination Kit for Susceptibility to Bioactive Substance X in Disease or Condition Associated with Bioactive Substance X on the Basis of Measurement of the Expression Level of Target Gene Y (Determination Kit IV)

The present invention provides a determination kit that enables the easy conduct of determination method IV.

This determination kit is referred to as “determination kit IV” as required.

In one embodiment, determination kit IV comprises the following (i) and (ii):
(i) a means capable of measuring the expression level of target gene Y;
(ii) a medium recording the relationship between the effect of bioactive substance X and the expression level of target gene Y.

The kit may further comprise a means capable of collecting of a biological sample from an animal, or a transcription product of target gene Y or target protein Y and the like.

The components of determination kit IV are the same as those of determination kit I except medium (ii).

The medium recording the relationship between the effect of bioactive substance X and the expression level of target gene Y can be one incorporating data on the correlation of the expression level of target gene Y and susceptibility to bioactive substance X. The expression level of target gene Y in an animal highly susceptible to bioactive substance X can increase (or decrease) compared to a less susceptible animal.

Determination kit IV enables the easy determination of susceptibility to bioactive substance X. Hence, determination method IV is useful for, for example, the evaluation of an action of bioactive substance X on a particular animal and the like.

### 8.2. Determination Methods and Determination Kits for Susceptibility to Bioactive Substance X on the Basis of Measurement of Polymorphism of Target Gene Y

8.2.1. Determination Method for Susceptibility to Bioactive Substance X in Disease or Condition Associated with Bioactive Substance X on the Basis of Measurement of Polymorphism of Target Gene Y (Determination Method V)

The present invention provides a determination method for susceptibility to bioactive substance X in a disease or condition associated with bioactive substance X, which comprises measuring the polymorphism of target gene Y.

This determination method is referred to as “determination method V” as required.

In one embodiment, determination method V comprises the following steps (a) and (b):
(a) a step for measuring the polymorphism of target gene Y in a biological sample collected from an animal;
(b) a step for predicting the effect of bioactive substance X in a disease or condition associated with target gene Y on the basis of the presence or absence of a particular type of polymorphism.

The methodology comprising the above-described steps (a) to (b) is referred to as “methodology VIII” as required.

Step (a) of methodology VIII is the same as step (a) of methodology VII.

In step (b) of methodology VIII, the effect of bioactive substance X in a disease or condition associated with bioactive substance X is evaluated on the basis of the type of polymorphism of target gene Y. Specifically, first, the measured type of polymorphism of target gene Y is checked against data on the correlation of the type of polymorphism of target gene Y and susceptibility to bioactive substance X in a disease or condition associated with bioactive substance X. This correlation can be determined by a method known per se.

Next, from the result of the comparison, susceptibility to bioactive substance X in a disease or condition associated with bioactive substance X is estimated. The combination of bioactive substance X and target gene Y are the same as described above. It is known that in animals that are highly susceptible to a bioactive substance, a particular type of polymorphism is often observed in a target gene for the bioactive substance. Hence, it is possible to determine the susceptibility of an animal to bioactive substance X by analyzing polymorphism. For example, provided that bioactive substance X is a drug, the likelihood or unlikelihood of obtainment of desired effect of the drug, or the probability of onset of adverse reaction of a drug, can be determined.

Determination method V enables the easy determination of susceptibility to bioactive substance X in a disease or condition associated with bioactive substance X. Hence, determination method V is useful for, for example, the evaluation of an action of bioactive substance X in a disease or condition associated with bioactive substance X and the like.

8.2.2. Determination Kit for Susceptibility to Bioactive Substance X in Disease or Condition Associated with Bioactive Substance X on the Basis of Measurement of Polymorphism of Target Gene Y (Determination Kit V)

The present invention also provides a determination kit that enables the easy conduct of determination method V.

This determination kit is referred to as “determination kit V” as required.

In one embodiment, determination kit V comprises the following (i) and (ii):
(i) a means capable of measuring the polymorphism of target gene Y;
(ii) a medium recording the relationship between the effect of bioactive substance X and the polymorphism of gene Y.

The kit may further comprise a means capable of collecting a biological sample from an animal, or a nucleic acid that encodes target gene Y having a particular type of polymorphism, a nucleic acid that encodes target gene Y not having a particular type of polymorphism and the like.

The constituents of determination kit V are the same as those of determination kit II except medium (ii).

The medium recording the relationship between the effect of active substance X and the polymorphism of gene Y can be one incorporating data on the correlation of susceptibility to bioactive substance X in a disease or condition associated with bioactive substance X and the type of polymorphism of target gene Y. The type of polymorphism of target gene Y in animals that are highly susceptible to bioactive substance X in a disease or condition associated with bioactive substance X can be one that encodes a protein that is more (or less) bindable to bioactive substance X compared to a less susceptible animal.

Determination kit V enables a determination of susceptibility to bioactive substance X in a disease or condition associated with bioactive substance X. Hence, determination kit V is useful for, for example, the evaluation of an action of bioactive substance X in a disease or condition associated with bioactive substance X and the like.

8.2.3. Determination Method for Susceptibility to Bioactive Substance X in Disease or Condition Associated with Target Gene Y on the Basis of Measurement of Polymorphism of Target Gene Y (Determination Method VI)

The present invention provides a determination method for susceptibility to bioactive substance X in a disease or condition associated with target gene Y, which comprises measuring the polymorphism of target gene Y.

This determination method is referred to as “determination method VI” as required.

In one embodiment, determination method VI comprises the following steps (a) and (b):
(a) a step for measuring the type of polymorphism of target protein Y in a biological sample collected from an animal;
(b) a step for predicting the effect of bioactive substance X in a disease or condition associated with target gene Y on the basis of the presence or absence of a particular type of polymorphism.

In this determination method, the type of polymorphism used to determine the susceptibility is one that alters the ability of target protein Y to bind to bioactive substance X. Such type of polymorphism can be determined by a method known per se such as binding assay. Animals having a target gene comprising the type of polymorphism that potentiates or reduces the binding ability to the bioactive substance are thought to be highly (or poorly) susceptible to the bioactive substance; animals having a target gene comprising a type of polymorphism that reduces the binding ability are considered to be less (or more) susceptible. Hence, the susceptibility of an animal to bioactive substance X can be determined by analyzing such type of polymorphism.

The methodology comprising steps (a) and (b) above in determination method VI is the same as methodology VIII except for the type of polymorphism of target gene Y to be measured.

Determination method VI enables the easy determination of susceptibility to bioactive substance X in a disease or condition associated with bioactive substance X. Hence, determination method VI is useful for, for example, the evaluation of an action of bioactive substance X in a disease or condition associated with bioactive substance X and the like.

8.2.4. Determination Kit for Susceptibility to Bioactive Substance X in Disease or Condition Associated with Target Gene Y on the Basis of Measurement of Polymorphism of Target Gene Y (Determination Kit VI)

The present invention also provides a determination kit that enables the easy conduct of determination method VI.

This determination kit is referred to as “determination kit VI” as required.

In one embodiment, determination kit VI comprises the following (i) and (ii):
(i) a means capable of measuring the polymorphism of target gene Y;
(ii) a medium recording the relationship between a disease or condition associated with target gene Y and the polymorphism of target gene Y.

The kit may further comprise a means capable of collecting a biological sample from an animal, or a nucleic acid that encodes target gene Y having a particular type of polymorphism, a nucleic acid that encodes target gene Y not having a particular type of polymorphism and the like.

In determination kit VI, the type of polymorphism used to determine the risk of onset is one that alters the ability of target protein Y to bind to bioactive substance X. The type of polymorphism can be determined by a method known per se such as binding assay.

The components of determination kit VI are the same as those of determination kit V except for the type of polymorphism of target gene Y to be measured.

Determination kit VI enables a determination of susceptibility to bioactive substance X in a disease or condition associated with bioactive substance X. Hence, determination kit VI is useful for, for example, the evaluation of an action of bioactive substance X in a disease or condition associated with bioactive substance X and the like.

The disclosures in all publications mentioned herein, including patents and patent application specifications, are incorporated by reference herein to the extent that all of them have been given expressly.

The present invention is hereinafter described in more detail by means of the following examples, which, however, are not to be construed as limiting the technical scope of the present invention.

### EXAMPLES

### Reference Example 1

### Method of Expressing Proteins from Human Full-Length cDNA Clone Using Escherichia coli

BP-reaction was performed on human full-length cDNA clone and the cloning vector Gateway pDONR201 by the PCR cloning method using the Invitrogen Gateway system to yield an entry clone. LR-reaction was performed on this entry clone with the destination vector pDEST17 (Gateway System) and LR Clonase at 25° C. for 60 minutes to yield an expression plasmid. The Escherichia coli expressing protein was expressed with the N terminal fused with a His-tag. Escherichia coli competent cell BL2lstar(DE3)pLysS were transformed with this expression plasmid, a clone incorporating the expression vector was selected, and a frozen stock was prepared. The transformant was inoculated into LB medium and precultured, after which it was transferred into SB medium and cultured to induce the expression of IPTG, and the cells were stored frozen.

### Reference Example 2

### Method of Purifying Expressed Protein of Human Full-Length cDNA Clone

A human full-length cDNA clone was expressed as a protein with an N-terminal His tag. This clone was purified using BioRobot 8000 (Qiagen) or AKTA Crystal (Amersham). In the purification with BioRobot 8000, the expression-induced frozen stock cells in Reference Example 1 was thawed and lysed with lysozyme, after which the cells were affinity-purified using Ni-NTA Superflow 96 BioRobot Kit (Qiagen). In the purification with AKTA Crystal, affinity purification using a HisTrap HP column was followed by gel filtration purification using the Gel Filtration Column HiLoad 16/60 or a 10/30 Superdex 75 prep grade column. The purified fraction was used for interaction analysis after being subjected to SDS-PAGE to verify the estimated molecular weight and purity.

As for the protein for Biacore measurement, the harvested Escherichia coli was suspended in a lysis buffer [50 mM NaH₂PO₄ pH 8.0, 0.3M NaCl, 10 mM Imidazole, Bensozase, rLysozyme, complete EDTA free (Roche Diagnostics, cat no. 1873580)] and to disrupted by sonication (2 sec treatment+2 sec, 5 min, on ice). Ni-NTA-agarose was added to the cell rupture solution to be bound to His-tag protein and Ni-NTA-agarose was washed several times with NPI-30 buffer [50 mM NaH₂PO₄ pH 8.0, 0.3M NaCl, 30 mM Imidazole]. The purified recombinant protein was eluted from Ni-NTA-agarose with NPI-500 buffer [50 mM NaH₂PO₄ pH 8.0, 0.3M NaCl, 500 mM Imidazole] containing high concentration of imidazole, and dialyzed against PBS to remove imidazole. The obtained protein was measured for the concentration and for the purity by SDS-PAGE and stored at 4° C.

### Reference Example 3

### Method of Expression and Purification of Protein from Human Full Length cDNA Clone Using Bombyx mori pupa

A part of the protein was expressed and purified by utilizing the protein production by the commissioning service “Superworm” based on the Bombyx mori pupa expression system by KATAKURA INDUSTRIES CO., LTD. A gene having a Histag on the C-terminal was inserted into recombinant Baculovirus and inoculated to Bombyx mori pupa. Milled cells of the expressed Bombyx mori pupa was sonicated, and the centrifugation supernatant thereof was filtered and subjected to Ni-NTA resin or affinity purification in the same manner as with Escherichia coli expression product.

### Reference Example 4

### Method of Analyzing Human Protein-Drug Interactions Using Size Exclusion Chromatography

To analyze the interactions between commonly used drugs and proteins expressed from human full-length cDNA clones while keeping both the proteins and the compounds in non-modified, non-immobilized state, size exclusion chromatography (SEC) and mass spectrometry were used in combination (SEC-MS method). The specific procedures are shown below.

### Step 1

A solution of a single drug or a multiplicated compound solution comprising a mixture of a plurality of drugs (e.g., 8, 16, 24 kinds) was added to the protein purified in Reference Example 2.

### Step 2

The compound-protein mixture prepared in step 1 was subjected to chromatography using an SEC column, the compound and the protein were separated by SEC, and the compound that interacted with the bound compound or protein contained in the protein fraction was analyzed using a mass analyzer.

The purified protein standard was concentrated by ultrafiltration and subjected to buffer solution exchange, and finally concentrated to obtain a concentration of not less than 25 μM. The final buffer composition was a 10 mM ADA (N-(2-Acetamido)iminodiacetic acid) buffer (pH 6.5)-300 mM NaCl aqueous solution for a metal ion-free buffer, or a 10 mM ADA(N-(2-Acetamido)iminodiacetic acid) Buffer (pH 6.5)-300 mM NaCl-100 μm mineral ion cocktail (Ca(OAc)₂, Zn(OAc)₂.2H₂O, Cu(OAc)₂.H₂O, Co(OAc)₂.4H₂O, Mn(OAc)₂.4H₂O, Mg(OAc)₂.4H₂O, FeCl₃. 6H₂O) aqueous solution for a metal ion added buffer. A protein solution prepared with a metal ion added buffer was used for the interaction screening by the SEC-MS method. However, as for a part of the protein used for testing the concentration dependency of the interaction, protein solutions each prepared using metal ion added or free buffers were used respectively to confirm metal ion requirement of the interaction. Protein concentrations were measured using BCA Protein Assay (PIERCE) in consideration of the purity calculated by SDS-PAGE.

A solution of a single pharmaceutical compound at a concentration of 1.25 mM in DMSO (dimethyl sulfoxide) or a multiplied compound solution of a plurality (8, 16 or 24 kinds) of compounds in DMSO was prepared, and these solutions were used for interaction analysis. In reproducibility confirmation experiments or dose dependency determination experiments, a solution of various concentrations of a single compound in DMSO (dimethyl sulfoxide) was used.

Mass spectrometry was performed using LCQ DECA XP (Thermoelectron) or Q-TOFmicro (Micromass), equipped with an ESI probe. The LC pump used was Agilent 1100 (Yokogawa Analytical Systems), and the autosampler used was HTC-PAL (CTC Analytics) equipped with a cooling stacker. The SEC column used was a 384-well spin column.

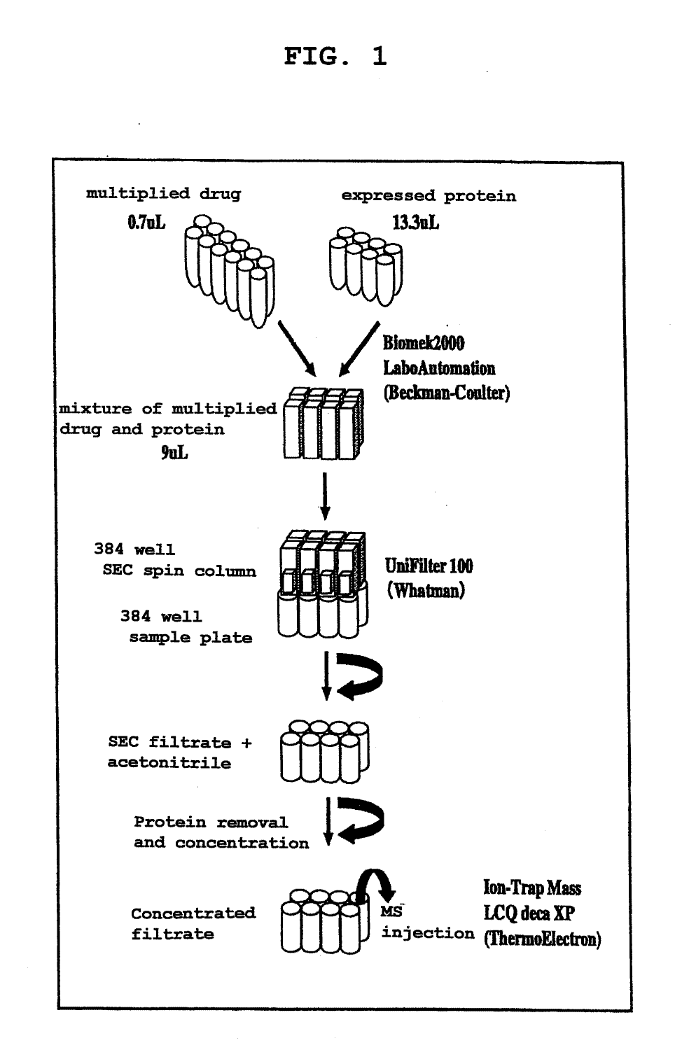

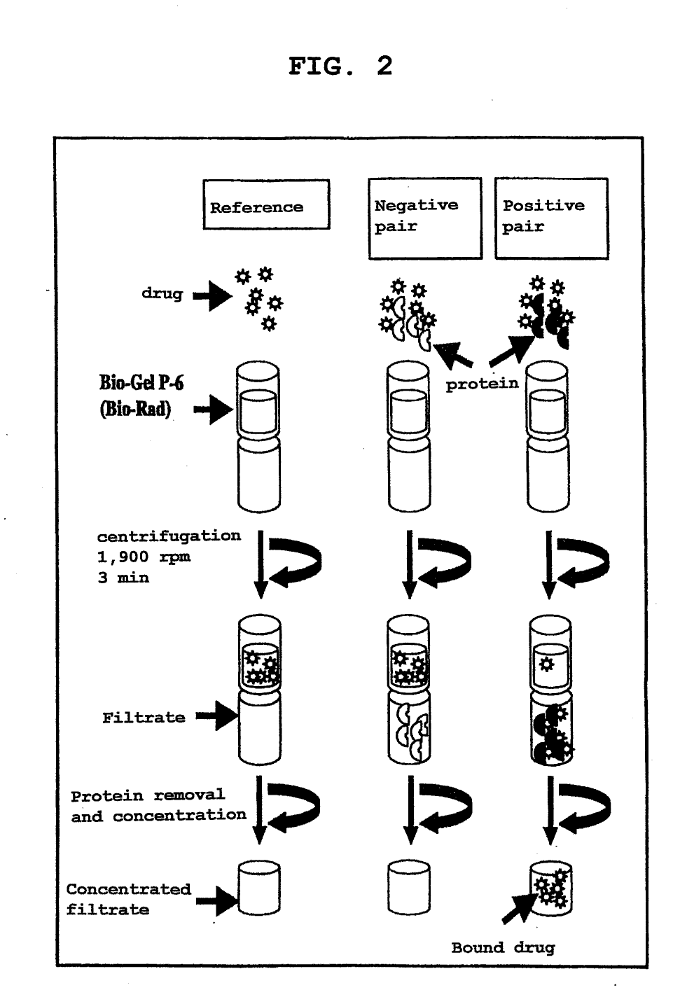

### Spin Column Method (FIGS. 1 and 2)

In the 384-well spin column method, Unifilter 100 (Whatman), packed with 10 μL (dry volume) of Bio-Gel P6 (BIO-RAD) and swollen with milliQ water, was used as the SEC column. 13.3 μL of a protein-free reference standard or a 25 μM protein standard and 0.7 μL of a multiplied liquid comprising 25 μM of each pharmaceutical compound (5% DMSO aqueous solution) were mixed; 9 μL of this mixture was aliquoted into the SEC spin columns. The SEC spin column was mounted on an acetonitrile-aliquoted 384-well U-bottom plate and centrifuged; the SEC spin column filtrate, which is a protein fraction, was retrieved in 50% acetonitrile. The protein precipitate produced by the acetonitrile was removed via centrifugation and filtration for deproteinization; the resulting filtrate was concentrated by centrifugation and re-dissolved in 10 μL of 50% methanol to obtain a mass spectrometry sample. The mobile phase supplied to the mass analyzer was 0.1% formic acid/50% methanol solution in the positive ion mode, and 0.1% ammonia/50% methanol solution in the negative ion mode; these mobile phases were used at a flow rate of 40 gL/min. 2-μL of mass spectrometry samples were injected using an autosampler at 2-minute intervals; the mass spectral intensity of the compound was measured to obtain the spectral intensity of the pharmaceutical compound contained in the SEC spin column filtrate (protein fraction eluted from SEC). The protein and the compound were judged to have interacted with each other if the spectral intensity of the compound in a mass spectrometry sample obtained from an SEC sample supplemented with a protein standard was greater than the spectral intensity of the compound in a mass spectrometry sample of reference SEC standard not supplemented with the protein. In the experiments for examining dose dependency, the protein and the compound were judged to have interacted with each other dose-dependently if the spectral intensity of the pharmaceutical compound contained in the SEC spin column filtrate (protein fraction eluted from SEC) increased as the compound concentration or/and protein concentration of the SEC sample was increased.

### Reference Example 5

### Measurement Dissociation Constant by BIACORE 3000

### Immobilization of Protein:

A protein was diluted with PBS to about 20 μg/mL-40 μg/mL, and immobilized on a CM5-Sensor chip, on which NTA had been immobilize by the affinity-amine-coupling method, or a commercially available NTA sensor chip.

In the affinity-amine-coupling method, 0.5 M NiCl₂ was injected for 1 min, EDC:NHS mixture (manufactured by BIACORE) was injected for 10 min to activate the sencor chip, after which a protein solution was injected continuously for. 10 min to 15 min for immobilization. After immobilization, 1M ethanolamine was injected for 7 min for deactivation. While the amount of the immobilized protein varies depending on the protein, it was about 6,800 RU on average with minimum 1,452 RU and maximum 16,655 RU.

### Dilution of Compound:

As the measurement buffer, Tris buffered Saline (10 mM Tris/HCl pH 7.4, 150 mM NaCl) (TBS) added with 2% DMSO was mainly used. For compound solubility and the like, PBS or HEPES buffered Saline (10 mM HEPES/HCl pH 7.4, 150 mM NaCl) (HBS) were also used. When a trace amount of metal ion was necessary for the property of protein-compound to be measured, 10 μM or 100 μM of calcium acetate, magnesium acetate and 1 μM of zinc acetate were added to the buffer before use. Because a compound often has low solubility, 0.005% of surfactant P-20 (manufactured by BIACORE), which is one kind of surfactant, was added.

The basic serial dilution of the compound included 6 stages of 100 μm, 33.3 μM, 11.1 μM, 3.7 μM, 1.23 μM, 0.41 μM, and the measurement was performed twice for 33.3 μM to confirm measurement reproducibility.

Particularly, when a Kd value not more than 1×10⁻⁵ M was obtained, the compound was diluted in 10 stages of 100 μM, 50 μM, 25 μM, 10 μM, 5 μM, 2.5 μM, 1 μM, 0.5 μM, 0.25 μM, 0.1 μM, and the measurement was performed twice for 100 μM, 50 μM, 25 μM, 10 μM, 5 μM, 2.5 μM, 1 μM, 0.5 μM to confirm measurement reproducibility.

When non-specific adsorption of a compound to a sensor surface is doubtful from general examination results, 1×10⁻⁴ M−1×10⁻³ M of ethanolamine was added to the measurement buffer and used for investigation.

For the measurement, BIACORE 3000 was used, and the compound was injected under KINJECT command. The flow rate was 50 μL/min, the injection was 3 min, and the dissociation was measured for 3 min thereafter.

After injection of the compound, the sensor surface was washed by successively injecting 10 mM HCl (6 sec), 1 mM NaOH (6 sec), 40 mM Octyl-glucose (10 sec). Where necessary, the washing operation was repeated.

### Amendment of Measurement Value and Calculation Method of Kd Value:

Before each measurement, DMSO was injected plural times to the measurement buffer at different concentrations (1.25%, 1.75%, 2.0%, 2.25%, 2.5%, 2.75% and the like), and the bulk effect was amended by DMSO (DMSO amendment) using the obtained value. Only the buffer used for dilution of the compound was injected, and used for the amendment of the noise and the like of the apparatus (0 amendment). The measurement results adjusted by DMSO amendment and 0 amendment were analyzed using BIA evaluation version 4.1. When the measurement results show a steady state binding at each dilution, steady state affinity was calculated to give Kd value. When dissociation is observed for several minutes after binding or when the steady state is not observed during compound injection, Kd value was calculated by Kinetics analysis (Simultaneous ka/kd, 1:1 binding model).

### Example 1

### Analysis of Interaction Between Expressed Protein and Compound (1)

Expression and purification of various proteins were performed according to the methods of Reference Examples 1 to 3, and the interactions between the various proteins and various compounds were analyzed according to the method of Reference Example 4. The pairs of various proteins and various compounds that showed interaction are shown in the following Tables 9-1 to 9-6.

TABLE 9-1

| SEQ ID NO: | FLJ No. | compound |
|---|---|---|
| 1 | FLJ21182 | Ajmaline |
| 1 | FLJ21182 | Celestin blue |
| 1 | FLJ21182 | Conessine |
| 1 | FLJ21182 | Diphenidol |
| 1 | FLJ21182 | Methoxy-6-harmalan |
| 1 | FLJ21182 | Pimethixene |
| 1 | FLJ21182 | Quinine |
| 1 | FLJ21182 | Ritodrine |
| 1 | FLJ21182 | Alimemazine |
| 1 | FLJ21182 | Boldine |
| 1 | FLJ21182 | Clofilium |
| 1 | FLJ21182 | Paroxetine |
| 1 | FLJ21182 | Trimethylcolchicinic acid |
| 1 | FLJ21182 | Antipyrine |
| 1 | FLJ21182 | Cephaeline |
| 1 | FLJ21182 | Ciclopirox |
| 1 | FLJ21182 | Coniine (DL) |
| 1 | FLJ21182 | Doxazosin |
| 1 | FLJ21182 | Sulfadimethoxine |
| 1 | FLJ21182 | Pantethine |
| 2 | FLJ38597 | Trimethylcolchicinic acid |
| 2 | FLJ38597 | Ajmaline |
| 2 | FLJ38597 | Celestin blue |
| 2 | FLJ38597 | Methoxy-6-harmalan |
| 2 | FLJ38597 | Minaprine |
| 2 | FLJ38597 | Ritodrine |
| 2 | FLJ38597 | Scoulerin |
| 2 | FLJ38597 | Alimemazine |
| 2 | FLJ38597 | Acetylcysteine |
| 3 | FLJ13700 | Celestin blue |
| 3 | FLJ13700 | Ciclopirox |
| 3 | FLJ13700 | Coniine (DL) |
| 3 | FLJ13700 | Tamoxifen |
| 3 | FLJ13700 | Acetylcysteine |
| 3 | FLJ13700 | Paracetamol |
| 4 | FLJ50683 | Molsidomine |
| 5 | FLJ50199 | Trimetazidine |
| 6 | FLJ26440 | Lobeline alpha (—) |
| 6 | FLJ26440 | Ebselen |
| 7 | FLJ21647 | Moroxidine |
| 8 | FLJ26620 | Xylazine |
| 9 | FLJ43792 | Terguride |

TABLE 9-2

| | | |
|---|---|---|
| 9 | FLJ43792 | Iobenguane |
| 10 | FLJ38127 | Quinine |
| 10 | FLJ38127 | Eburnamonine |
| 10 | FLJ38127 | Fluorocurarine |
| 10 | FLJ38127 | Furaltadone |
| 11 | FLJ35050 | Hydroflumethiazide |
| 12 | FLJ27298 | Methimazole |
| 13 | FLJ26262 | Risperidone |
| 14 | FLJ90682 | Bupivacaine |
| 15 | FLJ22923 | Loperamide |
| 15 | FLJ22923 | Clofazimine |
| 15 | FLJ22923 | Dipyridamole |
| 16 | FLJ22871 | Stanozolol |
| 16 | FLJ22871 | Methyclothiazide |
| 17 | FLJ20398 | Chromomycin A3 |
| 17 | FLJ20398 | Meclofenamic acid |
| 17 | FLJ20398 | Saquinavir |
| 18 | FLJ35377 | Promazine |
| 18 | FLJ35377 | Pranlukast |
| 19 | FLJ42145 | Dihydrostreptomycin |
| 19 | FLJ42145 | Iproniazide |
| 19 | FLJ42145 | Nefopam |
| 20 | FLJ26144 | Quercetine |
| 20 | FLJ26144 | Luteolin |
| 20 | FLJ26144 | Pranlukast |
| 21 | FLJ26374 | Pranlukast |
| 22 | FLJ26371 | Clemizole |
| 22 | FLJ26371 | Fenbendazole |
| 22 | FLJ26371 | Harmol |
| 22 | FLJ26371 | Ifosfamide |
| 22 | FLJ26371 | Piperlongumine |
| 22 | FLJ26371 | Propranolol |
| 23 | FLJ45688 | Acetohexamide |
| 23 | FLJ45688 | Benzethonium |
| 23 | FLJ45688 | Clomiphene |
| 23 | FLJ45688 | Cyclobenzaprine |
| 23 | FLJ45688 | Flupentixol |
| 23 | FLJ45688 | Guanfacine |
| 23 | FLJ45688 | Maprotiline |
| 23 | FLJ45688 | Perhexiline |
| 23 | FLJ45688 | Probenecid |
| 23 | FLJ45688 | Clinofibrate |
| 23 | FLJ45688 | Celecoxib |

TABLE 9-3

| | | |
|---|---|---|
| 23 | FLJ45688 | Gossypol |
| 23 | FLJ45688 | Althiazide |
| 23 | FLJ45688 | α-Ergocryptine |
| 23 | FLJ45688 | Gabexate |
| 23 | FLJ45688 | Clenbuterol |
| 23 | FLJ45688 | Etodolac |
| 23 | FLJ45688 | Misoprostol |
| 23 | FLJ45688 | Ubenimex |
| 23 | FLJ45688 | Acetohexamide |
| 23 | FLJ45688 | Clopamide |
| 23 | FLJ45688 | Glibenclamide |
| 23 | FLJ45688 | Glipizide |
| 23 | FLJ45688 | Isoxicam |
| 23 | FLJ45688 | Sulfaphenazole |
| 23 | FLJ45688 | Thioproperasine |
| 23 | FLJ45688 | Thiothixene (cis) |
| 23 | FLJ45688 | Tolbutamide |
| 23 | FLJ45688 | Methyclothiazide |
| 23 | FLJ45688 | Argatroban |
| 23 | FLJ45688 | Sulfadoxine |
| 23 | FLJ45688 | Sulfabenzamide |
| 23 | FLJ45688 | Benzthiazide |
| 23 | FLJ45688 | Valdecoxib |
| 24 | FLJ38620 | Acetohexamide |
| 24 | FLJ38620 | Isradipine |
| 24 | FLJ38620 | Mupirocin |
| 24 | FLJ38620 | Limaprost |
| 24 | FLJ38620 | Solasodine |
| 24 | FLJ38620 | Alacepril |
| 24 | FLJ38620 | Carboprost |
| 25 | FLJ26267 | Metergotamine |
| 25 | FLJ26267 | Methoxamine |
| 25 | FLJ26267 | Paroxetine |
| 25 | FLJ26267 | Dizocilpine |
| 25 | FLJ26267 | Fluvoxamine |
| 25 | FLJ26267 | 3-Hydroxykynurenine |
| 25 | FLJ26267 | Nimetazepam |
| 25 | FLJ26267 | Fludroxy cortide |
| 26 | FLJ26062 | Fenoprofen |
| 27 | FLJ22936 | Acenocoumarol |
| 27 | FLJ22936 | Budesonide |
| 27 | FLJ22936 | Chlorogenic acid |
| 27 | FLJ22936 | Chlorothiazide |

TABLE 9-4

| | | |
|---|---|---|
| 27 | FLJ22936 | Diclofenac |
| 27 | FLJ22936 | Diperodon |
| 27 | FLJ22936 | DO 897/99 |
| 27 | FLJ22936 | Nimesulide |
| 27 | FLJ22936 | Thioproperasine |
| 27 | FLJ22936 | Sarpogrelate |
| 28 | FLJ43223 | Acetylsalicylsalicylic acid |
| 29 | FLJ26102 | Buspirone |
| 30 | FLJ25218 | Dopamine |
| 30 | FLJ25218 | Alpha-methyl-5-hydroxytryptamine |
| 31 | FLJ45675 | Cisapride |
| 32 | FLJ25918 | Berberine |
| 32 | FLJ25918 | Celestin blue |
| 32 | FLJ25918 | Diflunisal |
| 32 | FLJ25918 | Mebendazole |
| 32 | FLJ25918 | Tranilast |
| 33 | FLJ46709 | Bromperidol |
| 33 | FLJ46709 | Coralyne |
| 34 | RGNpc017 | DO 897/99 |
| 34 | RGNpc017 | Domperidone |
| 34 | RGNpc017 | Flupentixol |
| 34 | RGNpc017 | Fluphenazine |
| 34 | RGNpc017 | L-thyroxine |
| 34 | RGNpc017 | Trifluoperazine |
| 34 | RGNpc017 | Clinofibrate |
| 34 | RGNpc017 | Acetohexamide |
| 34 | RGNpc017 | Chromomycin A3 |
| 34 | RGNpc017 | Carboprost |
| 35 | FLJ40377 | Alfuzocin |
| 35 | FLJ40377 | Clobetasone |
| 35 | FLJ40377 | Doxazosin |
| 35 | FLJ40377 | Pranlukast |
| 35 | FLJ40377 | Risperidone |
| 36 | FLJ25845 | Acetopromazine |
| 36 | FLJ25845 | Cyclopentolate |
| 36 | FLJ25845 | Perhexiline |
| 36 | FLJ25845 | Phenformin |
| 36 | FLJ25845 | Pyrilamine |
| 36 | FLJ25845 | Terconazole |
| 36 | FLJ25845 | Tobramycin |
| 36 | FLJ25845 | Amoxapine |
| 36 | FLJ25845 | Cephaeline |
| 36 | FLJ25845 | Clenbuterol |

TABLE 9-5

| | | |
|---|---|---|
| 36 | FLJ25845 | Domperidone |
| 36 | FLJ25845 | Minocycline |
| 36 | FLJ25845 | Moxalactam |
| 37 | FLJ23662 | Glibenclamide |
| 37 | FLJ23662 | Raloxifene |
| 37 | FLJ23662 | Clofazimine |
| 38 | FLJ12668 | Albendazole |
| 39 | FLJ90085 | Bezafibrate |
| 40 | FLJ90364 | Pirenzepine |
| 41 | FLJ90401 | Rescinnamine |
| 42 | FLJ25526 | Benzbromarone |
| 42 | FLJ25526 | Pranlukast |
| 42 | FLJ25526 | Mifepristone |
| 42 | FLJ25526 | Mestanolone |
| 43 | FLJ46896 | Hydroxytacrine (R,S) |
| 43 | FLJ46896 | Metergotamine |
| 43 | FLJ46896 | Metaproterenol |
| 44 | FLJ46856 | Eburnamonine |
| 44 | FLJ46856 | Levobunolol |
| 45 | FLJ90345 | Norharman |
| 45 | FLJ90345 | Pyrilamine |
| 46 | FLJ26550 | Celestin blue |
| 46 | FLJ26550 | Nitrarine |
| 47 | FLJ90015 | Clemizole |
| 48 | FLJ39454 | Clobazam |
| 49 | FLJ45115 | Josamycin |
| 49 | FLJ45115 | Oxytocin |
| 49 | FLJ45115 | Clarithromycin |
| 50 | FLJ90066 | Leuprolide |
| 50 | FLJ90066 | Cyclosporin A |
| 51 | FLJ37995 | Diclofenamide |
| 51 | FLJ37995 | Benzthiazide |
| 52 | FLJ26058 | Hydroxychloroquine |
| 52 | FLJ26058 | Furazolidone |
| 52 | FLJ26058 | Metanephrine (D, L) |
| 53 | FLJ46369 | Benzbromarone |
| 53 | FLJ46369 | Benzethonium |
| 53 | FLJ46369 | Clofazimine |
| 53 | FLJ46369 | Domperidone |
| 53 | FLJ46369 | Doxazosin |
| 53 | FLJ46369 | Gramicidin |
| 53 | FLJ46369 | α-Ergocryptine |
| 53 | FLJ46369 | Bicalutamide |

TABLE 9-6

| | | |
|---|---|---|
| 53 | FLJ46369 | Rescinnamine |
| 53 | FLJ46369 | Saquinavir |
| 53 | FLJ46369 | Syrosingopine |
| 53 | FLJ46369 | Pranlukast |
| 54 | FLJ16517 | Benzbromarone |
| 54 | FLJ16517 | Clofazimine |
| 54 | FLJ16517 | Domperidone |
| 54 | FLJ16517 | Nicardipine |
| 54 | FLJ16517 | Quercetine |
| 54 | FLJ16517 | Ebastine |
| 54 | FLJ16517 | Actinomycin D |
| 54 | FLJ16517 | Loperamide |
| 54 | FLJ16517 | Pranlukast |
| 54 | FLJ16517 | Luteolin |
| 55 | FLJ26591 | Pyrithyldione |
| 56 | FLJ26596 | Chlordiazepoxide |
| 56 | FLJ26596 | Flumequine |
| 57 | FLJ90480 | Buformin |
| 57 | FLJ90480 | 6-Furfurylaminopurine |
| 57 | FLJ90480 | Nitrarine |
| 57 | FLJ90480 | Pempidine |
| 58 | FLJ43067 | Viloxazine |
| 59 | FLJ25460 | Cefazolin |
| 59 | FLJ25460 | Fenbufen |
| 59 | FLJ25460 | Ketoprofen |
| 59 | FLJ25460 | Colchicine |
| 59 | FLJ25460 | Doxycycline |
| 59 | FLJ25460 | Gabapentin |
| 59 | FLJ25460 | Lidoflazine |
| 59 | FLJ25460 | Probenecid |
| 60 | FLJ26806 | Benzydamine |
| 60 | FLJ26806 | Clenbuterol |
| 61 | FLJ43911 | Benzethonium |
| 61 | FLJ43911 | Fluphenazine |
| 61 | FLJ43911 | GBR 12909 |
| 61 | FLJ43911 | Doxazosin |
| 61 | FLJ43911 | Procaine |
| 61 | FLJ43911 | Quinacrine |
| 62 | FLJ44715 | Azithromycin |
| 62 | FLJ44715 | Colistin |
| 63 | FLJ90031 | Protriptyline |
| 63 | FLJ90031 | Maprotiline |

In addition, the interaction of a part of the pairs from the above-mentioned pairs was tested for the concentration dependency by the method of Reference Example 4. A pair that shows an increase in the spectrum intensity of a pharmaceutical compound contained in a filtrate (protein elution fraction from SEC) of SEC spin column, in a manner dependent on the doses of the both of each low-molecular-weight compound and protein, is considered to show a concentration dependent interaction. The detail of the pair that showed concentration dependent interaction by the SEC-MS method is shown in the following Tables. In the following Tables, Mineral(+) means use of a protein standard product prepared using a metal ion added buffer, i.e., 10 mM ADA Buffer (pH 6.5)-300 mM NaCl-100 μM mineral ion cocktail (Ca(OAc)₂, Zn(OAc)₂.2H₂O, Cu(OAc)₂.H₂O, Co(OAc)₂.4H₂O, Mn(OAc)₂.4H₂O, Mg(OAc)₂.4H₂O, FeCl₃.6H₂O) aqueous solution. On the other hand, Mineral(−) means use of a protein standard product prepared using a metal ion-free buffer, i.e., 10 mM ADA Buffer (pH 6.5)-300 mM NaCl aqueous solution, as a comparative test to examine whether the interaction requires metal ion.

TABLE 10A
Minerals (−)
measured Mass Range: m/z = 326.4-327.9

| | | protein concentration (uM) | | |
|---|---|---|---|---|
| FLJ21182 - Ajmaline | | 0 | 11.9 | 23.8 |
| compound | 0 | 0.0 | 0.0 | 0.0 |
| concentration | 1 | 0.0 | 0.0 | 0.1 |
| (uM) | 10 | 0.2 | 0.6 | 0.4 |
| | 100 | 3.4 | 5.2 | 5.7 |
| | 250 | 9.3 | 11.8 | 15.2 |

TABLE 10B
Minerals (+)
measured Mass Range: m/z = 326.4-327.9

| | | protein concentration (uM) | | |
|---|---|---|---|---|
| FLJ21182 - Ajmaline | | 0 | 11.9 | 23.8 |
| compound | 0 | 0.0 | 0.0 | 0.0 |
| concentration | 1 | 0.1 | 0.0 | 0.0 |
| (uM) | 10 | 0.3 | 0.4 | 0.1 |
| | 100 | 3.5 | 4.0 | 3.2 |
| | 250 | 12.1 | 11.9 | 8.1 |

TABLE 11A
Minerals (−)
measured Mass Range: m/z = 328.4-329.9

| | | protein concentration (uM) | | |
|---|---|---|---|---|
| FLJ21182 - Celestin blue | | 0 | 11.9 | 23.8 |
| compound | 0 | 0.0 | 0.0 | 0.0 |
| concentration | 1 | −0.1 | 0.0 | 0.0 |
| (uM) | 10 | 0.0 | 0.1 | 0.2 |
| | 100 | 0.5 | 2.3 | 2.6 |
| | 250 | 0.8 | 4.8 | 6.7 |

TABLE 11B
Minerals (+)
measured Mass Range: m/z = 328.4-329.9

| | | protein concentration (uM) | | |
|---|---|---|---|---|
| FLJ21182 - Celestin blue | | 0 | 11.9 | 23.8 |
| compound | 0 | 0.0 | 0.0 | 0.0 |
| concentration | 1 | −0.2 | −0.1 | 0.0 |
| (uM) | 10 | 0.1 | 0.5 | 0.6 |
| | 100 | 3.5 | 5.5 | 7.2 |
| | 250 | 4.4 | 16.5 | 16.8 |

TABLE 12A
Minerals (−)
measured Mass Range: m/z = 356.6-358.1

| | | protein concentration (uM) | | |
|---|---|---|---|---|
| FLJ21182 - Conessine | | 0 | 11.9 | 23.8 |
| compound | 0 | 0.0 | 0.0 | 0.0 |
| concentration | 1 | 0.0 | 0.0 | 0.0 |
| (uM) | 10 | 0.2 | 0.2 | 0.2 |
| | 100 | 1.7 | 3.0 | 5.2 |
| | 250 | 7.6 | 9.8 | 12.1 |

TABLE 12B
Minerals (+)
measured Mass Range: m/z = 356.6-358.1

| | | protein concentration (uM) | | |
|---|---|---|---|---|
| FLJ21182 - Conessine | | 0 | 11.9 | 23.8 |
| compound | 0 | 0.0 | 0.0 | 0.0 |
| concentration | 1 | 0.0 | 0.0 | 0.1 |
| (uM) | 10 | 0.3 | 0.4 | 0.3 |
| | 100 | 3.5 | 3.0 | 4.3 |
| | 250 | 5.0 | 10.9 | 9.4 |

TABLE 13A
Minerals (−)
measured Mass Range: m/z = 309.4-310.9

| | | protein concentration (uM) | | |
|---|---|---|---|---|
| FLJ21182 - Diphenidol | | 0 | 11.9 | 23.8 |
| compound | 0 | 0.0 | 0.0 | 0.0 |
| concentration | 1 | 0.1 | 0.1 | 0.1 |
| (uM) | 10 | 0.9 | 0.4 | 1.9 |
| | 100 | 6.4 | 7.6 | 14.6 |
| | 250 | 13.5 | 31.2 | 34.1 |

TABLE 13B
Minerals (+)
measured Mass Range: m/z = 309.4-310.9

| | | protein concentration (uM) | | |
|---|---|---|---|---|
| FLJ21182 - Diphenidol | | 0 | 11.9 | 23.8 |
| compound | 0 | 0.0 | 0.0 | 0.0 |
| concentration | 1 | 0.0 | 0.0 | 0.1 |
| (uM) | 10 | 0.7 | 1.0 | 0.6 |
| | 100 | 5.9 | 10.1 | 10.2 |
| | 250 | 15.3 | 16.7 | 16.6 |

TABLE 14A
Minerals (−)
measured Mass Range: m/z = 214.3-215.8

| | | protein concentration (uM) | | |
|---|---|---|---|---|
| FLJ21182 - Methoxy-6-harmalan | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.2 | 0.3 |
| | 100 | 1.1 | 3.0 | 2.8 |
| | 250 | 1.7 | 3.7 | 4.7 |

TABLE 14B
Minerals (+)
measured Mass Range: m/z = 214.3-215.8

| | | protein concentration (uM) | | |
|---|---|---|---|---|
| FLJ21182 - Methoxy-6-harmalan | | 0 | 11.9 | 23.8 |
| compound | 0 | 0.0 | 0.0 | 0.0 |
| concentration | 1 | −0.1 | 0.0 | 0.0 |
| (uM) | 10 | 0.0 | 0.3 | 0.2 |
| | 100 | 1.2 | 3.2 | 2.5 |
| | 250 | 4.9 | 6.9 | 7.3 |

TABLE 15A
Minerals (−)
measured Mass Range: m/z = 293.4-294.9

| | | protein concentration (uM) | | |
|---|---|---|---|---|
| FLJ21182 - Pimethixene | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 0.5 | 2.3 | 2.3 |
| | 250 | 1.7 | 5.7 | 7.0 |

TABLE 15B
Minerals (+)
measured Mass Range: m/z = 293.4-294.9

| | | protein concentration (uM) | | |
|---|---|---|---|---|
| FLJ21182 - Pimethixene | | 0 | 11.9 | 23.8 |
| compound | 0 | 0.0 | 0.0 | 0.0 |
| concentration | 1 | 0.0 | 0.0 | 0.0 |
| (uM) | 10 | 0.1 | 0.2 | 0.2 |
| | 100 | 1.3 | 2.7 | 3.3 |
| | 250 | 3.1 | 10.0 | 11.7 |

TABLE 16A
Minerals (−)
measured Mass Range: m/z = 324.4-325.9

| | | protein concentration (uM) | | |
|---|---|---|---|---|
| FLJ21182 - Quinine | | 0 | 11.9 | 23.8 |
| compound | 0 | 0.0 | 0.0 | 0.0 |
| concentration | 1 | 0.1 | 0.0 | 0.0 |
| (uM) | 10 | 0.1 | 0.3 | 0.3 |
| | 100 | 2.0 | 5.0 | 4.7 |
| | 250 | 4.8 | 6.4 | 9.9 |

TABLE 16B
Minerals (+)
measured Mass Range: m/z = 324.4-325.9

| | | protein concentration (uM) | | |
|---|---|---|---|---|
| FLJ21182 - Quinine | | 0 | 11.9 | 23.8 |
| compound | 0 | 0.0 | 0.0 | 0.0 |
| concentration | 1 | 0.0 | 0.0 | 0.0 |
| (uM) | 10 | 0.2 | 0.3 | 0.2 |
| | 100 | 2.6 | 2.8 | 1.7 |
| | 250 | 5.6 | 6.7 | 7.4 |

TABLE 17A
Minerals (−)
measured Mass Range: m/z = 287.4-288.9

| | | protein concentration (uM) | | |
|---|---|---|---|---|
| FLJ21182 - Ritodrine | | 0 | 11.9 | 23.8 |
| compound | 0 | 0.0 | 0.0 | 0.0 |
| concentration | 1 | 0.0 | 0.1 | 0.0 |
| (uM) | 10 | 0.2 | 0.2 | 0.2 |
| | 100 | 3.3 | 5.8 | 5.8 |
| | 250 | 8.6 | 4.6 | 14.2 |

TABLE 17B
Minerals (+)
measured Mass Range: m/z = 287.4-288.9

| | | protein concentration (uM) | | |
|---|---|---|---|---|
| FLJ21182 - Ritodrine | | 0 | 11.9 | 23.8 |
| compound | 0 | 0.0 | 0.0 | 0.0 |
| concentration | 1 | 0.0 | 0.0 | 0.0 |
| (uM) | 10 | 0.1 | 0.2 | 0.3 |
| | 100 | 2.6 | 3.9 | 3.3 |
| | 250 | 6.4 | 9.3 | 8.0 |

TABLE 18A
Minerals (−)
measured Mass Range: m/z = 298.5-300

| FLJ21182 - Alimemazine | | protein concentration (uM) | | |
|---|---|---|---|---|
| (Trimeprazine) | | 0 | 11.9 | 23.8 |
| compound | 0 | 0.0 | 0.0 | 0.0 |
| concentration | 1 | 0.1 | 0.1 | 0.0 |
| (uM) | 10 | 0.2 | 0.3 | 0.7 |
| | 100 | 2.5 | 4.7 | 5.0 |
| | 250 | 6.6 | 8.7 | 13.4 |

TABLE 18B
Minerals (+)
measured Mass Range: m/z = 298.5-300

| FLJ21182 - Alimemazine | | protein concentration (uM) | | |
|---|---|---|---|---|
| (Trimeprazine) | | 0 | 11.9 | 23.8 |
| compound | 0 | 0.0 | 0.0 | 0.0 |
| concentration | 1 | 0.0 | 0.0 | 0.1 |
| (uM) | 10 | 0.1 | 0.1 | 0.2 |
| | 100 | 2.2 | 5.7 | 5.6 |
| | 250 | 8.5 | 13.9 | 8.2 |

TABLE 19A
Minerals (−)
measured Mass Range: m/z = 327.4-328.9

| | | protein concentration (uM) | | |
|---|---|---|---|---|
| FLJ21182 - Boldine | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 0.1 | 1.5 | 0.7 |
| | 250 | 0.4 | 3.2 | 1.6 |

TABLE 19B
Minerals (+)
measured Mass Range: m/z = 327.4-328.9

| | | protein concentration (uM) | | |
|---|---|---|---|---|
| FLJ21182 - Boldine | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.3 | 0.6 | 0.9 |
| | 250 | 1.7 | 2.3 | 2.1 |

TABLE 20A
Minerals (−)
measured Mass Range: m/z = 339-340.5

| | | protein concentration (uM) | | |
|---|---|---|---|---|
| FLJ21182 - Clofilium | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 3.7 | 7.4 | 5.5 |
| | 250 | 4.1 | 15.5 | 10.2 |

TABLE 20B
Minerals (+)
measured Mass Range: m/z = 339-340.5

| | | protein concentration (uM) | | |
|---|---|---|---|---|
| FLJ21182 - Clofilium | | 0 | 11.9 | 23.8 |
| compound | 0 | 0.0 | 0.0 | 0.0 |
| concentration | 1 | −0.1 | 0.0 | −0.1 |
| (uM) | 10 | 0.0 | 0.0 | 0.0 |
| | 100 | 8.0 | 7.3 | 7.1 |
| | 250 | 21.6 | 25.7 | 27.5 |

TABLE 21A
Minerals (−)
measured Mass Range: m/z = 329.4-330.9

| | | protein concentration (uM) | | |
|---|---|---|---|---|
| FLJ21182 - Paroxetine | | 0 | 11.9 | 23.8 |
| compound | 0 | 0.0 | 0.0 | 0.0 |
| concentration | 1 | 0.0 | 0.0 | 0.0 |
| (uM) | 10 | 0.1 | 0.2 | 0.1 |
| | 100 | 0.9 | 3.6 | 2.5 |
| | 250 | 3.3 | 6.7 | 7.2 |

TABLE 21B
Minerals (+)
measured Mass Range: m/z = 329.4-330.9

| | | protein concentration (uM) | | |
|---|---|---|---|---|
| FLJ21182 - Paroxetine | | 0 | 11.9 | 23.8 |
| compound | 0 | 0.0 | 0.0 | 0.0 |
| concentration | 1 | 0.1 | −0.1 | 0.1 |
| (uM) | 10 | 0.4 | 0.2 | 0.6 |
| | 100 | 6.9 | 5.4 | 9.0 |
| | 250 | 20.0 | 31.0 | 33.1 |

TABLE 22
Minerals (+)
measured Mass Range: m/z = 266.3-267.8

| | | protein concentration (uM) | | |
|---|---|---|---|---|
| FLJ50199 - Trimetazidine | | 0 | 23.8 | 47.5 |
| compound | 0 | 0.0 | 0.0 | 0.0 |
| concentration | 1 | −0.4 | 0.0 | −0.7 |
| (uM) | 10 | −0.3 | −0.7 | −0.8 |
| | 100 | 1.4 | 0.8 | 0.2 |
| | 250 | 6.7 | 11.5 | 11.2 |

TABLE 23
Minerals (+)
measured Mass Range: m/z = 337.5-339

| FLJ26440 - α-Lobeline (−) | | protein concentration (uM) | | |
|---|---|---|---|---|
| (Lobeline alpha (−)) | | 0 | 23.8 | 47.5 |
| compound | 0 | 0.0 | 0.0 | 0.0 |
| concentration | 1 | 0.0 | 0.2 | 0.2 |
| (uM) | 10 | 0.3 | 1.3 | 0.7 |
| | 100 | 2.0 | 14.3 | 20.5 |
| | 250 | 9.0 | 33.3 | 34.6 |

TABLE 24
Minerals (+)
measured Mass Range: m/z = 274.2-275.7

| | | protein concentration (uM) | | |
|---|---|---|---|---|
| FLJ26440 - Ebselen | | 0 | 23.8 | 47.5 |
| compound | 0 | 0.0 | 0.0 | 0.0 |
| concentration | 1 | 0.3 | 0.4 | 0.0 |
| (uM) | 10 | 1.2 | 0.1 | −0.3 |
| | 100 | 2.3 | 7.1 | 3.4 |
| | 250 | 2.7 | 4.4 | 22.0 |

TABLE 25
Minerals (+)
measured Mass Range: m/z = 171.2-172.7

| | | protein concentration (uM) | | |
|---|---|---|---|---|
| FLJ21647 - Moroxidine | | 0 | 23.8 | 47.5 |
| compound | 0 | 0.0 | 0.0 | 0.0 |
| concentration | 1 | −19.8 | −16.1 | 2.8 |
| (uM) | 10 | −15.1 | −10.8 | 5.7 |
| | 100 | −12.4 | 2.6 | 28.3 |
| | 250 | 9.9 | 24.9 | 40.7 |

TABLE 26A
Minerals (−)
measured Mass Range: m/z = 220.3-221.8

| | | protein concentration (uM) | | |
|---|---|---|---|---|
| FLJ26620 - Xylazine | | 0 | 23.8 | 47.5 |
| compound | 0 | 0.0 | 0.0 | 0.0 |
| concentration | 1 | −0.2 | 0.0 | 0.0 |
| (uM) | 10 | 0.0 | 0.1 | 0.3 |
| | 100 | 4.8 | 6.9 | 7.6 |
| | 250 | 15.7 | 10.2 | 15.7 |

TABLE 26B
Minerals (+)
measured Mass Range: m/z = 220.3-221.8

| | | protein concentration (uM) | | |
|---|---|---|---|---|
| FLJ26620 - Xylazine | | 0 | 23.8 | 47.5 |
| compound | 0 | 0.0 | 0.0 | 0.0 |
| concentration | 1 | −0.1 | 0.0 | 0.2 |
| (uM) | 10 | 0.5 | 0.8 | 0.9 |
| | 100 | 18.7 | 14.8 | 17.8 |
| | 250 | 23.9 | 40.2 | 40.4 |

TABLE 27A
Minerals (−)
measured Mass Range: m/z = 340.5-342

| | | protein concentration (uM) | | |
|---|---|---|---|---|
| FLJ43792 - Terguride | | 0 | 23.8 | 47.5 |
| compound | 0 | 0.0 | 0.0 | 0.0 |
| concentration | 1 | 0.0 | 0.1 | 0.1 |
| (uM) | 10 | 0.3 | 0.3 | 1.2 |
| | 100 | 4.7 | 7.6 | 10.2 |
| | 250 | 14.3 | 18.3 | 28.0 |

TABLE 27B
Minerals (+)
measured Mass Range: m/z = 340.5-342

| | | protein concentration (uM) | | |
|---|---|---|---|---|
| FLJ43792 - Terguride | | 0 | 23.8 | 47.5 |
| compound | 0 | 0.0 | 0.0 | 0.0 |
| concentration | 1 | 0.0 | 0.0 | 0.1 |
| (uM) | 10 | 0.4 | 0.9 | 0.8 |
| | 100 | 5.0 | 11.0 | 12.2 |
| | 250 | 20.2 | 30.2 | 42.4 |

TABLE 28A
Minerals (−)
measured Mass Range: m/z = 324.4-325.9

| | | protein concentration (uM) | | |
|---|---|---|---|---|
| FLJ38127 - Quinine | | 0 | 23.8 | 47.5 |
| compound | 0 | 0.0 | 0.0 | 0.0 |
| concentration | 1 | 0.0 | 0.0 | 0.1 |
| (uM) | 10 | 0.2 | 0.3 | 0.3 |
| | 100 | 1.5 | 1.4 | 1.8 |
| | 250 | 1.0 | 5.2 | 5.4 |

TABLE 28B
Minerals (+)
measured Mass Range: m/z = 324.4-325.9

| | | protein concentration (uM) | | |
|---|---|---|---|---|
| FLJ38127 - Quinine | | 0 | 23.8 | 47.5 |
| compound | 0 | 0.0 | 0.0 | 0.0 |
| concentration | 1 | 0.1 | 0.2 | 0.2 |
| (uM) | 10 | −0.1 | 0.0 | 0.1 |
| | 100 | 1.9 | 2.3 | 2.5 |
| | 250 | 3.8 | 6.2 | 7.8 |

TABLE 29A
Minerals (−)
measured Mass Range: m/z = 294.4-295.9

| FLJ38127 - Eburnamonine | | protein concentration (uM) | | |
|---|---|---|---|---|
| (Eburnamonine (−)) | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 1.1 | 1.2 | 2.4 |
| | 250 | 3.3 | 4.6 | 3.5 |

TABLE 29B
Minerals (+)
measured Mass Range: m/z = 294.4-295.9

| FLJ38127 - Eburnamonine | | protein concentration (uM) | | |
|---|---|---|---|---|
| (Eburnamonine (−)) | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.0 |
| | 100 | 1.1 | 1.7 | 2.1 |
| | 250 | 4.5 | 6.7 | 6.0 |

TABLE 30A
Minerals (−)
measured Mass Range: m/z = 307.4-308.9

| FLJ38127 - Fluorocurarine | | protein concentration (uM) | | |
|---|---|---|---|---|
| (Fluorocurarine chloride) | | 0 | 23.8 | 47.5 |
| compound | 0 | 0.0 | 0.0 | 0.0 |
| concentration | 1 | 0.0 | 0.0 | 0.1 |
| (uM) | 10 | 0.5 | 1.1 | 2.2 |
| | 100 | 9.5 | 9.6 | 5.9 |
| | 250 | 14.1 | 42.2 | 34.2 |

TABLE 30B
Minerals (+)
measured Mass Range: m/z = 307.4-308.9

| FLJ38127 - Fluorocurarine | | protein concentration (uM) | | |
|---|---|---|---|---|
| (Fluorocurarine chloride) | | 0 | 23.8 | 47.5 |
| compound | 0 | 0.0 | 0.0 | 0.0 |
| concentration | 1 | 0.0 | 0.1 | 0.1 |
| (uM) | 10 | 0.9 | 1.3 | 1.1 |
| | 100 | 10.6 | 24.9 | 21.4 |
| | 250 | 30.0 | 17.8 | 55.2 |

TABLE 31A
Minerals (−)
measured Mass Range: m/z = 324.3-325.8

| FLJ38127 - Furaltadone | | protein concentration (uM) | | |
|---|---|---|---|---|
| (Furaltadone hydrochloride) | | 0 | 23.8 | 47.5 |
| compound | 0 | 0.0 | 0.0 | 0.0 |
| concentration | 1 | 0.1 | 0.0 | 0.3 |
| (uM) | 10 | 0.5 | 0.6 | 0.2 |
| | 100 | 5.2 | 4.2 | 3.4 |
| | 250 | 12.0 | 11.3 | 14.2 |

TABLE 31B
Minerals (+)
measured Mass Range: m/z = 324.3-325.8

| FLJ38127 - Furaltadone | | protein concentration (uM) | | |
|---|---|---|---|---|
| (Furaltadone hydrochloride) | | 0 | 23.8 | 47.5 |
| compound | 0 | 0.0 | 0.0 | 0.0 |
| concentration | 1 | −0.2 | 0.3 | 0.9 |
| (uM) | 10 | −0.7 | −0.5 | 0.2 |
| | 100 | 1.9 | 7.4 | 2.5 |
| | 250 | 18.8 | 16.4 | 25.2 |

TABLE 32A
Minerals (−)
measured Mass Range: m/z = 331.3-332.8

| | | protein concentration (uM) | | |
|---|---|---|---|---|
| FLJ35050 - Hydroflumethiazide | | 0 | 23.8 | 47.5 |
| compound | 0 | 0.0 | 0.0 | 0.0 |
| concentration | 1 | 0.1 | 0.1 | 0.1 |
| (uM) | 10 | 0.2 | 0.5 | 0.8 |
| | 100 | 1.5 | 3.0 | 8.1 |
| | 250 | 4.4 | 8.7 | 12.8 |

TABLE 32B
Minerals (+)
measured Mass Range: m/z = 331.3-332.8

| | | protein concentration (uM) | | |
|---|---|---|---|---|
| FLJ35050 - Hydroflumethiazide | | 0 | 23.8 | 47.5 |
| compound | 0 | 0.0 | 0.0 | 0.0 |
| concentration | 1 | 0.0 | 0.1 | 0.0 |
| (uM) | 10 | 0.1 | 0.5 | 1.0 |
| | 100 | 1.1 | 6.8 | 9.1 |
| | 250 | 5.9 | 11.7 | 13.1 |

TABLE 33
Minerals (+)
measured Mass Range: m/z = 114.2-115.7

| | | protein concentration (uM) | | |
|---|---|---|---|---|
| FLJ27298 - Methimazole | | 0 | 23.8 | 47.5 |
| compound | 0 | 0.0 | 0.0 | 0.0 |
| concentration | 1 | −1.8 | −0.3 | 2.1 |
| (uM) | 10 | −0.9 | −0.6 | 17.1 |
| | 100 | −1.2 | 2.0 | 17.0 |
| | 250 | 5.5 | 5.6 | 23.1 |

TABLE 34
Minerals (+)
measured Mass Range: m/z = 410.5-412

| | | protein concentration (uM) | | |
|---|---|---|---|---|
| FLJ26262 - Risperidone | | 0 | 23.8 | 47.5 |
| compound | 0 | 0.0 | 0.0 | 0.0 |
| concentration | 1 | 0.1 | 0.2 | 0.1 |
| (uM) | 10 | 1.4 | 2.3 | 1.2 |
| | 100 | 15.2 | 16.9 | 26.8 |
| | 250 | 23.5 | 41.4 | 43.0 |

TABLE 35A
Minerals (−)
measured Mass Range: m/z = 288.4-289.9

| | | protein concentration (uM) | | |
|---|---|---|---|---|
| FLJ90682 - Bupivacaine | | 0 | 23.8 | 47.5 |
| compound | 0 | 0.0 | 0.0 | 0.0 |
| concentration | 1 | 0.0 | 0.1 | 0.0 |
| (uM) | 10 | 0.4 | 1.1 | 1.3 |
| | 100 | 9.4 | 24.1 | 24.9 |
| | 250 | 39.2 | 60.5 | 68.6 |

TABLE 35B
Minerals (+)
measured Mass Range: m/z = 288.4-289.9

| | | protein concentration (uM) | | |
|---|---|---|---|---|
| FLJ90682 - Bupivacaine | | 0 | 23.8 | 47.5 |
| compound | 0 | 0.0 | 0.0 | 0.0 |
| concentration | 1 | 0.5 | 0.1 | 0.0 |
| (uM) | 10 | 1.3 | 2.1 | 1.4 |
| | 100 | 7.8 | 15.6 | 24.2 |
| | 250 | 14.8 | 43.5 | 41.4 |

TABLE 36A
Minerals (−)
measured Mass Range: m/z = 477-478.5

| | | protein concentration (uM) | | |
|---|---|---|---|---|
| FLJ22923 - Loperamide | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.2 | 0.8 |
| | 100 | 3.8 | 8.2 | 10.1 |
| | 250 | 17.1 | 23.7 | 36.2 |

TABLE 36B
Minerals (+)
measured Mass Range: m/z = 477-478.5

| | | protein concentration (uM) | | |
|---|---|---|---|---|
| FLJ22923 - Loperamide | | 0 | 23.8 | 47.5 |
| compound | 0 | 0.0 | 0.0 | 0.0 |
| concentration | 1 | 0.0 | 0.0 | 0.0 |
| (uM) | 10 | 0.2 | 0.4 | 0.2 |
| | 100 | 12.1 | 5.0 | 6.0 |
| | 250 | 1.3 | 23.3 | 20.3 |

TABLE 37A
Minerals (−)
measured Mass Range: m/z = 473.4-474.9

| | | protein concentration (uM) | | |
|---|---|---|---|---|
| FLJ22923 - Clofazimine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 0.0 | 0.2 | 9.1 |
| | 250 | 0.0 | 1.0 | 4.2 |

TABLE 37B
Minerals (+)
measured Mass Range: m/z = 473.4-474.9

| | | protein concentration (uM) | | |
|---|---|---|---|---|
| FLJ22923 - Clofazimine | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.0 | 0.0 |
| | 100 | −0.1 | 0.0 | 0.0 |
| | 250 | −0.1 | 0.0 | 0.0 |

TABLE 38A
Minerals (−)
measured Mass Range: m/z = 504.6-506.1

| | | protein concentration (uM) | | |
|---|---|---|---|---|
| FLJ22923 - Dipyridamole | | 0 | 23.8 | 47.5 |
| compound | 0 | 0.0 | 0.0 | 0.0 |
| concentration | 1 | 0.0 | 0.0 | 0.0 |
| (uM) | 10 | 0.2 | 0.2 | 0.7 |
| | 100 | 0.6 | 9.4 | 11.4 |
| | 250 | 0.3 | 12.2 | 13.8 |

TABLE 38B
Minerals (+)
measured Mass Range: m/z = 504.6-506.1

| | | protein concentration (uM) | | |
|---|---|---|---|---|
| FLJ22923 - Dipyridamole | | 0 | 23.8 | 47.5 |
| compound | 0 | 0.0 | 0.0 | 0.0 |
| concentration | 1 | 0.0 | 0.0 | 0.0 |
| (uM) | 10 | 0.2 | 0.2 | 0.2 |
| | 100 | 0.7 | 4.1 | 3.8 |
| | 250 | 0.4 | 6.6 | 1.7 |

TABLE 39A
Minerals (−)
measured Mass Range: m/z = 328.5-330

| | | protein concentration (uM) | | |
|---|---|---|---|---|
| FLJ22871 - Stanozolol | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 0.0 | 0.5 | 2.0 |
| | 250 | 0.0 | 2.2 | 2.3 |

TABLE 39B
Minerals (+)
measured Mass Range: m/z = 328.5-330

| | | protein concentration (uM) | | |
|---|---|---|---|---|
| FLJ22871 - Stanozolol | | 0 | 11.9 | 23.8 |
| compound | 0 | 0.0 | 0.0 | 0.0 |
| concentration | 1 | 0.0 | 0.0 | 0.0 |
| (uM) | 10 | −0.1 | 0.0 | 0.0 |
| | 100 | 0.0 | 1.2 | 4.5 |
| | 250 | 0.0 | 6.1 | 6.5 |

TABLE 40A
Minerals (−)
measured Mass Range: m/z = 360.2-361.7

protein concentration (uM)

| | | | |
|---|---|---|---|
| FLJ22871 - Methyclothiazide | 0 | 11.9 | 23.8 |

| | | | | |
|---|---|---|---|---|
| compound | 0 | 0.0 | 0.0 | 0.0 |
| concentration | 1 | 0.1 | 0.1 | 0.0 |
| (uM) | 10 | 0.3 | 0.4 | 0.2 |
| | 100 | 2.3 | 3.4 | 4.7 |
| | 250 | 3.6 | 3.2 | 4.0 |

TABLE 40B
Minerals (+)
measured Mass Range: m/z = 360.2-361.7

protein concentration (uM)

| | | | |
|---|---|---|---|
| FLJ22871 - Methyclothiazide | 0 | 11.9 | 23.8 |

| | | | | |
|---|---|---|---|---|
| compound | 0 | 0.0 | 0.0 | 0.0 |
| concentration | 1 | 0.1 | −0.1 | 0.0 |
| (uM) | 10 | 0.3 | 0.2 | 0.3 |
| | 100 | 2.1 | 2.5 | 4.7 |
| | 250 | 8.8 | 12.7 | 8.2 |

TABLE 41A
Minerals (−)
measured Mass Range: m/z = 1183.3-1184.8

protein concentration (uM)

| | | | |
|---|---|---|---|
| FLJ20398 - Chromomycin A3 | 0 | 23.8 | 47.5 |

| | | | | |
|---|---|---|---|---|
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 1.7 | 0.2 |
| | 250 | 20.1 | 29.3 | 70.8 |

TABLE 41B
Minerals (+)
measured Mass Range: m/z = 1183.3-1184.8

protein concentration (uM)

| | | | |
|---|---|---|---|
| FLJ20398 - Chromomycin A3 | 0 | 23.8 | 47.5 |

| | | | | |
|---|---|---|---|---|
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 0.0 | 0.7 |
| | 250 | 42.9 | 28.3 | 68.9 |

TABLE 42
Minerals (+)
measured Mass Range: m/z = 296.2-297.7

| | | protein concentration (uM) | | |
|---|---|---|---|---|
| FLJ20398 - Meclofenamic acid | | 0 | 23.8 | 47.5 |
| compound | 0 | 0.0 | 0.0 | 0.0 |
| concentration | 1 | 0.0 | −1.4 | 3.1 |
| (uM) | 10 | 1.7 | 0.6 | 4.2 |
| | 100 | 2.5 | 3.9 | 8.9 |
| | 250 | −0.3 | 3.0 | 9.2 |

TABLE 43A
Minerals (−)
measured Mass Range: m/z = 670.9-672.4

| | | protein concentration (uM) | | |
|---|---|---|---|---|
| FLJ20398 - Saquinavir | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 1.1 | 1.9 | 1.4 |
| | 250 | 3.6 | 4.2 | 4.2 |

TABLE 43B
Minerals (+)
measured Mass Range: m/z = 670.9-672.4

| | | protein concentration (uM) | | |
|---|---|---|---|---|
| FLJ20398 - Saquinavir | | 0 | 23.8 | 47.5 |
| compound | 0 | 0.0 | 0.0 | 0.0 |
| concentration | 1 | 0.0 | 0.1 | 0.1 |
| (uM) | 10 | 0.0 | 0.0 | 0.0 |
| | 100 | 1.7 | 2.1 | 1.3 |
| | 250 | 0.4 | 3.4 | 6.9 |

TABLE 44A
Minerals (−)
measured Mass Range: m/z = 284.4-285.9

| FLJ35377 - Promazine | | protein concentration (uM) | | |
|---|---|---|---|---|
| (Promazine hydrochloride) | | 0 | 23.8 | 47.5 |
| compound | 0 | 0.0 | 0.0 | 0.0 |
| concentration | 1 | −0.1 | 0.0 | 0.0 |
| (uM) | 10 | 0.0 | 1.0 | 0.7 |
| | 100 | 8.4 | 9.8 | 17.8 |
| | 250 | 12.5 | 15.7 | 34.9 |

TABLE 44B
Minerals (+)
measured Mass Range: m/z = 284.4-285.9

| FLJ35377 - Promazine | | protein concentration (uM) | | |
|---|---|---|---|---|
| (Promazine hydrochloride) | | 0 | 23.8 | 47.5 |
| compound | 0 | 0.0 | 0.0 | 0.0 |
| concentration | 1 | −0.6 | 0.0 | 0.1 |
| (uM) | 10 | −0.5 | 0.1 | 0.2 |
| | 100 | −0.3 | 6.3 | 10.6 |
| | 250 | 5.6 | 0.3 | 16.3 |

TABLE 45A
Minerals (−)
measured Mass Range: m/z = 481.5-483

| | | protein concentration (uM) | | |
|---|---|---|---|---|
| FLJ35377 - Pranlukast | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 1.0 | 3.0 |
| | 250 | 0.1 | 3.5 | 0.7 |

TABLE 45B
Minerals (+)
measured Mass Range: m/z = 481.5-483

| | | protein concentration (uM) | | |
|---|---|---|---|---|
| FLJ35377 - Pranlukast | | 0 | 23.8 | 47.5 |
| compound | 0 | 0.0 | 0.0 | 0.0 |
| concentration | 1 | −1.4 | −0.3 | −0.1 |
| (uM) | 10 | −1.5 | −0.3 | −0.1 |
| | 100 | −1.5 | 3.0 | 5.1 |
| | 250 | −1.3 | −0.3 | 0.4 |

TABLE 46A
Minerals (−)
measured Mass Range: m/z = 320.3-321.8

| | | protein concentration (uM) | | |
|---|---|---|---|---|
| FLJ26144 - Quercetine | | 0 | 23.8 | 47.5 |
| compound | 0 | 0.0 | 0.0 | 0.0 |
| concentration | 1 | 0.1 | 0.3 | 4.4 |
| (uM) | 10 | 0.4 | 0.2 | 0.2 |
| | 100 | 0.6 | 0.1 | 0.1 |
| | 250 | 0.2 | 0.2 | 0.1 |

TABLE 46B
Minerals (+)
measured Mass Range: m/z = 320.3-321.8

| | | protein concentration (uM) | | |
|---|---|---|---|---|
| FLJ26144 - Quercetine | | 0 | 23.8 | 47.5 |
| compound | 0 | 0.0 | 0.0 | 0.0 |
| concentration | 1 | 0.0 | 0.9 | −1.7 |
| (uM) | 10 | 0.8 | 0.2 | −3.3 |
| | 100 | −0.2 | 5.6 | 1.1 |
| | 250 | −0.1 | 22.7 | 70.9 |

TABLE 47A
Minerals (−)
measured Mass Range: m/z = 286.2-287.7

| | | protein concentration (uM) | | |
|---|---|---|---|---|
| FLJ26144 - Luteolin | | 0 | 23.8 | 47.5 |
| compound | 0 | 0.0 | 0.0 | 0.0 |
| concentration | 1 | 0.0 | 0.0 | 0.1 |
| (uM) | 10 | 0.0 | 0.0 | 0.0 |
| | 100 | 0.1 | 0.1 | 0.1 |
| | 250 | 0.0 | 0.5 | 0.1 |

TABLE 47B
Minerals (+)
measured Mass Range: m/z = 286.2-287.7

| | | protein concentration (uM) | | |
|---|---|---|---|---|
| FLJ26144 - Luteolin | | 0 | 23.8 | 47.5 |
| compound | 0 | 0.0 | 0.0 | 0.0 |
| concentration | 1 | −0.3 | −0.1 | 0.1 |
| (uM) | 10 | −0.3 | 0.0 | 0.1 |
| | 100 | −0.3 | 41.3 | 41.3 |
| | 250 | 0.0 | 62.7 | 85.6 |

TABLE 48A
Minerals (−)
measured Mass Range: m/z = 481.5-483

| | | protein concentration (uM) | | |
|---|---|---|---|---|
| FLJ26144 - Pranlukast | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 2.6 | 10.8 |
| | 250 | 0.0 | 0.1 | 30.8 |

TABLE 48B
Minerals (+)
measured Mass Range: m/z = 481.5-483

| | | protein concentration (uM) | | |
|---|---|---|---|---|
| FLJ26144 - Pranlukast | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.0 | 0.0 |
| | 100 | −0.1 | 0.9 | 4.8 |
| | 250 | −0.1 | 0.6 | 13.1 |

TABLE 49A
Minerals (−)
measured Mass Range: m/z = 481.5-483

| | | protein concentration (uM) | | |
|---|---|---|---|---|
| FLJ26374 - Pranlukast | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.4 |
| | 100 | 0.0 | 26.2 | 24.5 |
| | 250 | 0.0 | 2.1 | 5.8 |

TABLE 49B
Minerals (+)
measured Mass Range: m/z = 481.5-483

| | | protein concentration (uM) | | |
|---|---|---|---|---|
| FLJ26374 - Pranlukast | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.3 | 0.6 |
| | 100 | −0.1 | 7.0 | 18.9 |
| | 250 | −0.1 | 0.9 | 64.3 |

TABLE 50A
Minerals (−)
measured Mass Range: m/z = 325.8-327.3

| | | protein concentration (uM) | | |
|---|---|---|---|---|
| FLJ26371 - Clemizole | | 0 | 23.8 | 47.5 |
| compound | 0 | 0.0 | 0.0 | 0.0 |
| concentration | 1 | 0.1 | 0.2 | 0.3 |
| (uM) | 10 | 0.2 | 1.4 | 2.2 |
| | 100 | 2.8 | 13.3 | 19.8 |
| | 250 | 5.6 | 19.1 | 21.5 |

TABLE 50B
Minerals (+)
measured Mass Range: m/z = 325.8-327.3

| | | protein concentration (uM) | | |
|---|---|---|---|---|
| FLJ26371 - Clemizole | | 0 | 23.8 | 47.5 |
| compound | 0 | 0.0 | 0.0 | 0.0 |
| concentration | 1 | 0.2 | 0.2 | 0.1 |
| (uM) | 10 | 0.2 | 0.3 | 1.2 |
| | 100 | 0.6 | 10.8 | 22.1 |
| | 250 | 1.1 | 31.9 | 56.5 |

TABLE 51
Minerals (+)
measured Mass Range: m/z = 299.4-300.9

| | | protein concentration (uM) | | |
|---|---|---|---|---|
| FLJ26371 - Fenbendazole | | 0 | 23.8 | 47.5 |
| compound | 0 | 0.0 | 0.0 | 0.0 |
| concentration | 1 | 0.0 | 0.3 | 0.4 |
| (uM) | 10 | 0.2 | 3.0 | 2.3 |
| | 100 | 0.0 | 2.7 | 5.5 |
| | 250 | 0.1 | 5.3 | 6.7 |

TABLE 52A
Minerals (−)
measured Mass Range: m/z = 198.2-199.7

| | | protein concentration (uM) | | |
|---|---|---|---|---|
| FLJ26371 - Harmol | | 0 | 23.8 | 47.5 |
| compound | 0 | 0.0 | 0.0 | 0.0 |
| concentration | 1 | −1.3 | 0.2 | 0.0 |
| (uM) | 10 | −1.6 | 0.1 | 0.2 |
| | 100 | −1.3 | 2.0 | 2.0 |
| | 250 | −1.2 | 1.0 | 4.0 |

TABLE 52B
Minerals (+)
measured Mass Range: m/z = 198.2-199.7

| | | protein concentration (uM) | | |
|---|---|---|---|---|
| FLJ26371 - Harmol | | 0 | 23.8 | 47.5 |
| compound | 0 | 0.0 | 0.0 | 0.0 |
| concentration | 1 | −0.4 | −0.4 | 0.2 |
| (uM) | 10 | −0.5 | −0.3 | 0.0 |
| | 100 | 0.0 | −0.6 | 1.7 |
| | 250 | −0.1 | −0.3 | 5.4 |

TABLE 53A
Minerals (−)
measured Mass Range: m/z = 261.1-262.6

| | | protein concentration (uM) | | |
|---|---|---|---|---|
| FLJ26371 - Ifosfamide | | 0 | 23.8 | 47.5 |
| compound | 0 | 0.0 | 0.0 | 0.0 |
| concentration | 1 | −0.3 | 0.0 | 0.0 |
| (uM) | 10 | 0.6 | 0.6 | 0.5 |
| | 100 | 4.4 | 9.2 | 14.7 |
| | 250 | 21.9 | 27.9 | 32.3 |

TABLE 53B
Minerals (+)
measured Mass Range: m/z = 261.1-262.6

| | | protein concentration (uM) | | |
|---|---|---|---|---|
| FLJ26371 - Ifosfamide | | 0 | 23.8 | 47.5 |
| compound | 0 | 0.0 | 0.0 | 0.0 |
| concentration | 1 | −0.3 | 0.0 | 0.0 |
| (uM) | 10 | 0.6 | 0.7 | 0.6 |
| | 100 | 4.2 | 9.0 | 14.7 |
| | 250 | 22.4 | 27.8 | 32.5 |

TABLE 54A
Minerals (−)
measured Mass Range: m/z = 317.3-318.8

| | | protein concentration (uM) | | |
|---|---|---|---|---|
| FLJ26371 - Piperlongumine | | 0 | 23.8 | 47.5 |
| compound | 0 | 0.0 | 0.0 | 0.0 |
| concentration | 1 | −0.6 | −0.1 | −0.1 |
| (uM) | 10 | −0.4 | 0.0 | 0.0 |
| | 100 | 1.0 | 2.4 | 1.8 |
| | 250 | 2.8 | 3.5 | 8.4 |

TABLE 54B
Minerals (+)
measured Mass Range: m/z = 317.3-318.8

| | | protein concentration (uM) | | |
|---|---|---|---|---|
| FLJ26371 - Piperlongumine | | 0 | 23.8 | 47.5 |
| compound | 0 | 0.0 | 0.0 | 0.0 |
| concentration | 1 | −1.0 | −0.5 | −0.2 |
| (uM) | 10 | −1.1 | −0.3 | −0.2 |
| | 100 | 0.1 | 1.0 | 1.5 |
| | 250 | −0.3 | 3.9 | 11.1 |

TABLE 55A
Minerals (−)
measured Mass Range: m/z = 259.4-260.9

| | | protein concentration (uM) | | |
|---|---|---|---|---|
| FLJ26371 - Propranolol | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.6 | 1.9 |
| | 100 | 1.8 | 4.8 | 10.2 |
| | 250 | 5.5 | 12.9 | 22.1 |

TABLE 55B
Minerals (+)
measured Mass Range: m/z = 259.4-260.9

| | | protein concentration (uM) | | |
|---|---|---|---|---|
| FLJ26371 - Propranolol | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.7 | 3.2 |
| | 100 | 4.1 | 8.9 | 7.9 |
| | 250 | 6.2 | 6.6 | 26.8 |

TABLE 56A
Minerals (−)
measured Mass Range: m/z = 324.4-325.9

| | | protein concentration (uM) | | |
|---|---|---|---|---|
| FLJ45688 - Acetohexamide | | 0 | 11.9 | 23.8 |
| compound | 0 | 0.0 | 0.0 | 0.0 |
| concentration | 1 | 0.1 | 0.1 | 0.1 |
| (uM) | 10 | 0.4 | 0.7 | 0.5 |
| | 100 | 3.3 | 4.8 | 6.0 |
| | 250 | 8.8 | 9.2 | 14.2 |

TABLE 56B
Minerals (+)
measured Mass Range: m/z = 324.4-325.9

| | | protein concentration (uM) | | |
|---|---|---|---|---|
| FLJ45688 - Acetohexamide | | 0 | 11.9 | 23.8 |
| compound | 0 | 0.0 | 0.0 | 0.0 |
| concentration | 1 | 0.0 | 0.0 | 0.0 |
| (uM) | 10 | 0.5 | 0.1 | 0.6 |
| | 100 | 5.4 | 2.2 | 7.5 |
| | 250 | 10.7 | 7.4 | 10.8 |

TABLE 57A
Minerals (−)
measured Mass Range: m/z = 412.6-414.1

| | | protein concentration (uM) | | |
|---|---|---|---|---|
| FLJ45688 - Benzethonium | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.3 | 0.6 |
| | 100 | 0.0 | 0.1 | 19.4 |
| | 250 | 0.4 | 24.4 | 41.9 |

TABLE 57B
Minerals (+)
measured Mass Range: m/z = 412.6-414.1

| | | protein concentration (uM) | | |
|---|---|---|---|---|
| FLJ45688 - Benzethonium | | 0 | 11.9 | 23.8 |
| compound | 0 | 0.0 | 0.0 | 0.0 |
| concentration | 1 | −0.1 | 0.0 | 0.1 |
| (uM) | 10 | −0.1 | 0.3 | 1.2 |
| | 100 | 0.8 | 8.7 | 22.1 |
| | 250 | 12.4 | 40.9 | 57.6 |

TABLE 58A
Minerals (−)
measured Mass Range: m/z = 406-407.5

| | | protein concentration (uM) | | |
|---|---|---|---|---|
| FLJ45688 - Clomiphene | | 0 | 11.9 | 23.8 |
| compound | 0 | 0.0 | 0.0 | 0.0 |
| concentration | 1 | 0.1 | 0.0 | 0.1 |
| (uM) | 10 | 0.1 | 0.0 | 0.4 |
| | 100 | 0.1 | 3.7 | 9.3 |
| | 250 | 0.0 | 8.9 | 31.7 |

TABLE 58B
Minerals (+)
measured Mass Range: m/z = 406-407.5

| | | protein concentration (uM) | | |
|---|---|---|---|---|
| FLJ45688 - Clomiphene | | 0 | 11.9 | 23.8 |
| compound | 0 | 0.0 | 0.0 | 0.0 |
| concentration | 1 | −0.1 | 0.0 | 0.0 |
| (uM) | 10 | 0.0 | 0.0 | 0.5 |
| | 100 | 0.0 | 3.9 | 8.2 |
| | 250 | 0.5 | 15.5 | 33.2 |

TABLE 59A
Minerals (−)
measured Mass Range: m/z = 275.4-276.9

protein concentration (uM)

| | | | |
|---|---|---|---|
| FLJ45688 - Cyclobenzaprine | 0 | 11.9 | 23.8 |

| | | | | |
|---|---|---|---|---|
| compound | 0 | 0.0 | 0.0 | 0.0 |
| concentration | 1 | 0.0 | 0.0 | 0.0 |
| (uM) | 10 | 0.0 | 0.1 | 0.2 |
| | 100 | 1.6 | 2.3 | 2.6 |
| | 250 | 4.5 | 6.8 | 12.0 |

TABLE 59B
Minerals (+)
measured Mass Range: m/z = 275.4-276.9

protein concentration (uM)

| | | | |
|---|---|---|---|
| FLJ45688 - Cyclobenzaprine | 0 | 11.9 | 23.8 |

| | | | | |
|---|---|---|---|---|
| compound | 0 | 0.0 | 0.0 | 0.0 |
| concentration | 1 | 0.0 | 0.0 | 0.0 |
| (uM) | 10 | 0.1 | 0.3 | 0.4 |
| | 100 | 0.4 | 3.4 | 4.9 |
| | 250 | 4.9 | 12.1 | 14.7 |

TABLE 60A
Minerals (−)
measured Mass Range: m/z = 434.5-436

| FLJ45688 - Flupentixol | | protein concentration (uM) | | |
|---|---|---|---|---|
| (Flupentixol (Z)) | | 0 | 11.9 | 23.8 |
| compound | 0 | 0.0 | 0.0 | 0.0 |
| concentration | 1 | 0.0 | −0.1 | 0.0 |
| (uM) | 10 | 0.0 | 0.0 | 0.1 |
| | 100 | 0.1 | 0.0 | 2.3 |
| | 250 | 0.6 | 5.7 | 13.1 |

TABLE 60B
Minerals (+)
measured Mass Range: m/z = 434.5-436

| FLJ45688 - Flupentixol | | protein concentration (uM) | | |
|---|---|---|---|---|
| (Flupentixol (Z)) | | 0 | 11.9 | 23.8 |
| compound | 0 | 0.0 | 0.0 | 0.0 |
| concentration | 1 | −0.1 | 0.0 | −0.1 |
| (uM) | 10 | −0.1 | 0.0 | 0.1 |
| | 100 | 0.0 | 1.2 | 1.0 |
| | 250 | 0.3 | 5.1 | 7.4 |

TABLE 61A
Minerals (−)
measured Mass Range: m/z = 246.1-247.6

| | | protein concentration (uM) | | |
|---|---|---|---|---|
| FLJ45688 - Guanfacine | | 0 | 11.9 | 23.8 |
| compound | 0 | 0.0 | 0.0 | 0.0 |
| concentration | 1 | 0.0 | −0.1 | −0.1 |
| (uM) | 10 | −0.1 | −0.1 | 0.0 |
| | 100 | 0.7 | 2.2 | 2.9 |
| | 250 | 4.4 | 11.3 | 11.8 |

TABLE 61B
Minerals (+)
measured Mass Range: m/z = 246.1-247.6

| | | protein concentration (uM) | | |
|---|---|---|---|---|
| FLJ45688 - Guanfacine | | 0 | 11.9 | 23.8 |
| compound | 0 | 0.0 | 0.0 | 0.0 |
| concentration | 1 | −0.1 | −0.1 | −0.1 |
| (uM) | 10 | 0.1 | 0.0 | 0.2 |
| | 100 | 0.8 | 2.5 | 3.3 |
| | 250 | 2.5 | 8.9 | 10.0 |

TABLE 62A
Minerals (−)
measured Mass Range: m/z = 277.4-278.9

| | | protein concentration (uM) | | |
|---|---|---|---|---|
| FLJ45688 - Maprotiline | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.2 | 0.2 |
| | 100 | 1.8 | 3.2 | 4.1 |
| | 250 | 6.1 | 10.2 | 14.6 |

TABLE 62B
Minerals (+)
measured Mass Range: m/z = 277.4-278.9

| | | protein concentration (uM) | | |
|---|---|---|---|---|
| FLJ45688 - Maprotiline | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 3.0 | 4.2 | 4.0 |
| | 250 | 6.3 | 9.4 | 17.5 |

TABLE 63A
Minerals (−)
measured Mass Range: m/z = 277.6-279.1

| | | protein concentration (uM) | | |
|---|---|---|---|---|
| FLJ45688 - Perhexiline | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 1.4 | 2.0 | 5.7 |
| | 250 | 4.2 | 16.2 | 24.2 |

TABLE 63B
Minerals (+)
measured Mass Range: m/z = 277.6-279.1

| | | protein concentration (uM) | | |
|---|---|---|---|---|
| FLJ45688 - Perhexiline | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.2 |
| | 100 | 1.4 | 3.3 | 4.9 |
| | 250 | 7.0 | 13.8 | 21.6 |

TABLE 64A
Minerals (−)
measured Mass Range: m/z = 285.4-286.9

| | | protein concentration (uM) | | |
|---|---|---|---|---|
| FLJ45688 - Probenecid | | 0 | 11.9 | 23.8 |
| compound | 0 | 0.0 | 0.0 | 0.0 |
| concentration | 1 | −0.1 | 0.1 | 0.1 |
| (uM) | 10 | 0.5 | 0.4 | 0.8 |
| | 100 | 6.1 | 11.5 | 9.5 |
| | 250 | 14.2 | 38.7 | 28.8 |

TABLE 64B
Minerals (+)
measured Mass Range: m/z = 285.4-286.9

| | | protein concentration (uM) | | |
|---|---|---|---|---|
| FLJ45688 - Probenecid | | 0 | 11.9 | 23.8 |
| compound | 0 | 0.0 | 0.0 | 0.0 |
| concentration | 1 | −3.4 | 0.3 | −0.3 |
| (uM) | 10 | −2.3 | 1.2 | 1.1 |
| | 100 | 6.5 | 15.6 | 18.9 |
| | 250 | 37.5 | 40.3 | 42.5 |

TABLE 65A
Minerals (−)
measured Mass Range: m/z = 468.6-470.1

| | | protein concentration (uM) | | |
|---|---|---|---|---|
| FLJ45688 - Clinofibrate | | 0 | 11.9 | 23.8 |
| compound | 0 | 0.0 | 0.0 | 0.0 |
| concentration | 1 | 0.3 | 0.4 | 0.2 |
| (uM) | 10 | 0.1 | 1.4 | 1.6 |
| | 100 | 11.6 | 16.1 | 16.0 |
| | 250 | 18.9 | 39.5 | 44.5 |

TABLE 65B
Minerals (+)
measured Mass Range: m/z = 468.6-470.1

| | | protein concentration (uM) | | |
|---|---|---|---|---|
| FLJ45688 - Clinofibrate | | 0 | 11.9 | 23.8 |
| compound | 0 | 0.0 | 0.0 | 0.0 |
| concentration | 1 | −0.1 | 0.2 | 0.0 |
| (uM) | 10 | −0.1 | 0.9 | 1.0 |
| | 100 | 7.8 | 14.3 | 15.4 |
| | 250 | 27.0 | 45.6 | 43.4 |

TABLE 66A
Minerals (−)
measured Mass Range: m/z = 381.4-382.9

| | | protein concentration (uM) | | |
|---|---|---|---|---|
| FLJ45688 - Celecoxib | | 0 | 11.9 | 23.8 |
| compound | 0 | 0.0 | 0.0 | 0.0 |
| concentration | 1 | 0.1 | 0.1 | 0.0 |
| (uM) | 10 | 0.1 | 0.1 | 0.1 |
| | 100 | 0.0 | 0.3 | 0.8 |
| | 250 | 0.1 | 0.0 | 1.2 |

TABLE 66B
Minerals (+)
measured Mass Range: m/z = 381.4-382.9

| | | protein concentration (uM) | | |
|---|---|---|---|---|
| FLJ45688 - Celecoxib | | 0 | 11.9 | 23.8 |
| compound | 0 | 0.0 | 0.0 | 0.0 |
| concentration | 1 | −0.4 | 0.0 | 0.0 |
| (uM) | 10 | −0.2 | 0.0 | 0.0 |
| | 100 | −0.2 | 0.1 | 0.4 |
| | 250 | −0.3 | 0.4 | 2.6 |

TABLE 67A
Minerals (−)
measured Mass Range: m/z = 518.6-520.1

| | | protein concentration (uM) | | |
|---|---|---|---|---|
| FLJ45688 - Gossypol | | 0 | 11.9 | 23.8 |
| compound | 0 | 0.0 | 0.0 | 0.0 |
| concentration | 1 | 0.0 | 0.0 | 0.1 |
| (uM) | 10 | 0.0 | 0.5 | 0.6 |
| | 100 | 0.1 | 21.1 | 20.0 |
| | 250 | 0.2 | 44.2 | 116.3 |

TABLE 67B
Minerals (+)
measured Mass Range: m/z = 518.6-520.1

| | | protein concentration (uM) | | |
|---|---|---|---|---|
| FLJ45688 - Gossypol | | 0 | 11.9 | 23.8 |
| compound | 0 | 0.0 | 0.0 | 0.0 |
| concentration | 1 | −0.4 | −0.1 | 0.0 |
| (uM) | 10 | −0.5 | 0.3 | 0.1 |
| | 100 | −0.2 | 14.0 | 17.7 |
| | 250 | −0.3 | 26.7 | 52.2 |

TABLE 68A
Minerals (−)
measured Mass Range: m/z = 383.9-385.4

| | | protein concentration (uM) | | |
|---|---|---|---|---|
| FLJ45688 - Althiazide | | 0 | 11.9 | 23.8 |
| compound | 0 | 0.0 | 0.0 | 0.0 |
| concentration | 1 | −0.3 | 0.0 | 0.0 |
| (uM) | 10 | −0.3 | 0.0 | −0.1 |
| | 100 | 0.6 | 1.1 | 2.2 |
| | 250 | 1.4 | 3.8 | 1.8 |

TABLE 68B
Minerals (+)
measured Mass Range: m/z = 383.9-385.4

| | | protein concentration (uM) | | |
|---|---|---|---|---|
| FLJ45688 - Althiazide | | 0 | 11.9 | 23.8 |
| compound | 0 | 0.0 | 0.0 | 0.0 |
| concentration | 1 | −0.2 | −0.1 | 0.1 |
| (uM) | 10 | 0.1 | 0.1 | 0.3 |
| | 100 | 0.5 | 0.6 | 1.1 |
| | 250 | 1.8 | 1.5 | 7.2 |

TABLE 69A
Minerals (−)
measured Mass Range: m/z = 575.7-577.2

| FLJ45688 - α-Ergocryptine | | protein concentration (uM) | | |
|---|---|---|---|---|
| (Ergocryptine-alpha) | | 0 | 11.9 | 23.8 |
| compound | 0 | 0.0 | 0.0 | 0.0 |
| concentration | 1 | 0.0 | 0.1 | −0.1 |
| (uM) | 10 | 0.0 | 0.0 | −0.1 |
| | 100 | 0.4 | 2.3 | 1.1 |
| | 250 | 1.4 | 8.3 | 15.6 |

TABLE 69B

| FLJ45688 -α- | | protein | | |
|---|---|---|---|---|
| Ergocryptine | | concentration (uM) | | |
| (Ergocryptine-alpha) | | 0 | 11.9 | 23.8 |
| compound | 0 | 0.0 | 0.0 | 0.0 |
| concentration | 1 | 0.0 | −0.1 | 0.0 |
| (uM) | 10 | 0.0 | 0.1 | 0.2 |
| | 100 | 0.3 | 1.7 | 1.0 |
| | 250 | 1.4 | 14.2 | 16.7 |

Minerals (+)
measured Mass Range: m/z = 575.7-577.2

TABLE 70A

| | | protein | | |
|---|---|---|---|---|
| | | concentration (uM) | | |
| FLJ45688 - Gabexate | | 0 | 11.9 | 23.8 |
| compound | 0 | 0.0 | 0.0 | 0.0 |
| concentration | 1 | 0.0 | 0.0 | 0.0 |
| (uM) | 10 | 0.1 | 0.1 | 0.1 |
| | 100 | 1.6 | 0.9 | 1.5 |
| | 250 | 3.4 | 12.7 | 8.4 |

Minerals (−)
measured Mass Range: m/z = 321.4-322.9

TABLE 70B

| | | protein | | |
|---|---|---|---|---|
| | | concentration (uM) | | |
| FLJ45688 - Gabexate | | 0 | 11.9 | 23.8 |
| compound | 0 | 0.0 | 0.0 | 0.0 |
| concentration | 1 | −0.1 | 0.0 | 0.0 |
| (uM) | 10 | 0.0 | 0.1 | 0.1 |
| | 100 | 2.2 | 3.6 | 3.8 |
| | 250 | 7.1 | 10.7 | 13.1 |

Minerals (+)
measured Mass Range: m/z = 321.4-322.9

TABLE 71A

| | | protein | | |
|---|---|---|---|---|
| FLJ45688 - | | concentration (uM) | | |
| Clenbuterol | | 0 | 11.9 | 23.8 |
| compound | 0 | 0.0 | 0.0 | 0.0 |
| concentration | 1 | 0.0 | 0.1 | −0.1 |
| (uM) | 10 | 0.1 | 0.5 | 0.2 |
| | 100 | 3.4 | 5.5 | 5.1 |
| | 250 | 9.2 | 16.8 | 15.2 |

Minerals (−)
measured Mass Range: m/z = 277.2-278.7

TABLE 71B

| | | protein | | |
|---|---|---|---|---|
| FLJ45688 - | | concentration (uM) | | |
| Clenbuterol | | 0 | 11.9 | 23.8 |
| compound | 0 | 0.0 | 0.0 | 0.0 |
| concentration | 1 | 0.0 | 0.0 | 0.0 |
| (uM) | 10 | 0.5 | 0.7 | 0.6 |
| | 100 | 4.4 | 7.9 | 7.3 |
| | 250 | 13.2 | 19.2 | 16.4 |

Minerals (+)
measured Mass Range: m/z = 277.2-278.7

TABLE 72A

| | | protein | | |
|---|---|---|---|---|
| | | concentration (uM) | | |
| FLJ45688 - Etodolac | | 0 | 11.9 | 23.8 |
| compound | 0 | 0.0 | 0.0 | 0.0 |
| concentration | 1 | −0.3 | 0.0 | 0.0 |
| (uM) | 10 | 0.5 | 0.9 | 0.7 |
| | 100 | 4.6 | 10.8 | 12.3 |
| | 250 | 21.1 | 29.5 | 20.2 |

Minerals (−)
measured Mass Range: m/z = 287.4-288.9

TABLE 72B

| | | protein | | |
|---|---|---|---|---|
| | | concentration (uM) | | |
| FLJ45688 - Etodolac | | 0 | 11.9 | 23.8 |
| compound | 0 | 0.0 | 0.0 | 0.0 |
| concentration | 1 | −0.1 | 0.1 | 0.2 |
| (uM) | 10 | 0.2 | 0.5 | 0.6 |
| | 100 | 4.5 | 3.6 | 6.2 |
| | 250 | 8.6 | 6.5 | 8.3 |

Minerals (+)
measured Mass Range: m/z = 287.4-288.9

TABLE 73A

| | | protein | | |
|---|---|---|---|---|
| FLJ45688 - | | concentration (uM) | | |
| Misoprostol | | 0 | 11.9 | 23.8 |
| compound | 0 | 0.0 | 0.0 | 0.0 |
| concentration | 1 | −0.3 | 0.5 | −0.2 |
| (uM) | 10 | 0.6 | 2.0 | 1.2 |
| | 100 | 12.0 | 14.8 | 12.5 |
| | 250 | 30.5 | 41.9 | 37.8 |

Minerals (−)
measured Mass Range: m/z = 368.5-370

TABLE 73B

| | | protein | | |
|---|---|---|---|---|
| FLJ45688 - | | concentration (uM) | | |
| Misoprostol | | 0 | 11.9 | 23.8 |
| compound | 0 | 0.0 | 0.0 | 0.0 |
| concentration | 1 | 0.4 | −0.8 | 0.0 |
| (uM) | 10 | 0.1 | −0.8 | 0.9 |
| | 100 | 11.7 | 10.2 | 11.9 |
| | 250 | 38.1 | 11.1 | 22.7 |

Minerals (+)
measured Mass Range: m/z = 368.5-370

TABLE 74A

| | | protein | | |
|---|---|---|---|---|
| | | concentration (uM) | | |
| FLJ45688 - Ubenimex | | 0 | 11.9 | 23.8 |
| compound | 0 | 0.0 | 0.0 | 0.0 |
| concentration | 1 | −1.3 | −1.6 | −1.1 |
| (uM) | 10 | −1.5 | 1.9 | −0.3 |
| | 100 | 10.3 | 14.3 | 15.8 |
| | 250 | 29.4 | 33.0 | 31.2 |

Minerals (−)
measured Mass Range: m/z = 308.4-309.9

TABLE 74B

| | | protein | | |
|---|---|---|---|---|
| | | concentration (uM) | | |
| FLJ45688 - Ubenimex | | 0 | 11.9 | 23.8 |
| compound | 0 | 0.0 | 0.0 | 0.0 |
| concentration | 1 | −3.0 | −4.9 | 0.8 |
| (uM) | 10 | −2.7 | −3.4 | 2.0 |
| | 100 | 9.3 | 9.0 | 9.8 |
| | 250 | 26.4 | 23.4 | 19.0 |

Minerals (+)
measured Mass Range: m/z = 308.4-309.9

TABLE 75A

| | | protein | | |
|---|---|---|---|---|
| FLJ45688 - | | concentration (uM) | | |
| Acetohexamide | | 0 | 11.9 | 23.8 |
| compound | 0 | 0.0 | 0.0 | 0.0 |
| concentration | 1 | 0.1 | 0.1 | 0.1 |
| (uM) | 10 | 0.4 | 0.7 | 0.5 |
| | 100 | 3.3 | 4.8 | 6.0 |
| | 250 | 8.8 | 9.2 | 14.2 |

Minerals (−)
measured Mass Range: m/z = 324.4-325.9

TABLE 75B

| | | protein | | |
|---|---|---|---|---|
| FLJ45688 - | | concentration (uM) | | |
| Acetohexamide | | 0 | 11.9 | 23.8 |
| compound | 0 | 0.0 | 0.0 | 0.0 |
| concentration | 1 | 0.0 | 0.0 | 0.0 |
| (uM) | 10 | 0.5 | 0.1 | 0.6 |
| | 100 | 5.4 | 2.2 | 7.5 |
| | 250 | 10.7 | 7.4 | 10.8 |

Minerals (+)
measured Mass Range: m/z = 324.4-325.9

TABLE 76

| | | protein | | |
|---|---|---|---|---|
| FLJ38620 - | | concentration (uM) | | |
| Acetohexamide | | 0 | 11.9 | 23.8 |
| compound | 0 | 0.0 | 0.0 | 0.0 |
| concentration | 1 | −0.1 | 0.0 | 0.7 |
| (uM) | 10 | −0.1 | 0.5 | 1.1 |
| | 100 | 6.4 | 11.2 | 8.4 |
| | 250 | 15.9 | 14.0 | 19.4 |

Minerals (+)
measured Mass Range: m/z = 324.4-325.9

TABLE 77

| | | protein | | |
|---|---|---|---|---|
| | | concentration (uM) | | |
| FLJ38620 - Isradipine | | 0 | 11.9 | 23.8 |
| compound | 0 | 0.0 | 0.0 | 0.0 |
| concentration | 1 | 0.0 | 0.4 | 0.0 |
| (uM) | 10 | −0.2 | 0.4 | 0.0 |
| | 100 | 0.1 | 1.1 | 0.5 |
| | 250 | 0.5 | 1.5 | 3.5 |

Minerals (+)
measured Mass Range: m/z = 371.4-372.9

TABLE 78

| | | protein | | |
|---|---|---|---|---|
| | | concentration (uM) | | |
| FLJ38620 - Mupirocin | | 0 | 11.9 | 23.8 |
| compound | 0 | 0.0 | 0.0 | 0.0 |
| concentration | 1 | 0.0 | 0.1 | 0.1 |
| (uM) | 10 | 0.5 | 1.4 | 2.2 |
| | 100 | 9.5 | 14.2 | 17.0 |
| | 250 | 27.3 | 42.7 | 85.2 |

Minerals (+)
measured Mass Range: m/z = 500.6-502.1

TABLE 79

| | | protein | | |
|---|---|---|---|---|
| | | concentration (uM) | | |
| FLJ38620 - Limaprost | | 0 | 11.9 | 23.8 |
| compound | 0 | 0.0 | 0.0 | 0.0 |
| concentration | 1 | −0.6 | −0.7 | 0.1 |
| (uM) | 10 | −1.8 | 0.1 | 1.1 |
| | 100 | 7.4 | 12.8 | 11.9 |
| | 250 | 23.9 | 29.9 | 35.6 |

Minerals (+)
measured Mass Range: m/z = 380.5-382

TABLE 80

| | | protein | | |
|---|---|---|---|---|
| | | concentration (uM) | | |
| FLJ38620 - Solasodine | | 0 | 11.9 | 23.8 |
| compound | 0 | 0.0 | 0.0 | 0.0 |
| concentration | 1 | 0.0 | 0.1 | 0.1 |
| (uM) | 10 | 0.0 | 0.3 | 0.2 |
| | 100 | 0.0 | 0.2 | 0.5 |
| | 250 | 0.0 | 0.4 | 2.7 |

Minerals (+)
measured Mass Range: m/z = 413.6-415.1

TABLE 81

| | | protein | | |
|---|---|---|---|---|
| | | concentration (uM) | | |
| FLJ38620 - Alacepril | | 0 | 11.9 | 23.8 |
| compound | 0 | 0.0 | 0.0 | 0.0 |
| concentration | 1 | 0.3 | 0.1 | 0.4 |
| (uM) | 10 | 0.8 | 0.9 | 0.9 |
| | 100 | 9.0 | 10.0 | 13.4 |
| | 250 | 23.7 | 31.1 | 27.4 |

Minerals (+)
measured Mass Range: m/z = 406.5-408

TABLE 82

| | | protein | | |
|---|---|---|---|---|
| | | concentration (uM) | | |
| FLJ38620 - Carboprost | | 0 | 11.9 | 23.8 |
| compound | 0 | 0.0 | 0.0 | 0.0 |
| concentration | 1 | −0.1 | −0.1 | 0.0 |
| (uM) | 10 | 0.2 | 1.0 | 1.1 |
| | 100 | 10.3 | 13.0 | 9.7 |
| | 250 | 24.4 | 35.1 | 34.3 |

Minerals (+)
measured Mass Range: m/z = 368.5-370

TABLE 83A

| | | protein | | |
|---|---|---|---|---|
| FLJ26267 - Metergotamine | | concentration (uM) | | |
| (Metergoline) | | 0 | 9.5 | 19.0 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 0.2 | 1.4 | 1.0 |
| | 250 | 1.2 | 2.2 | 2.9 |

Minerals (−)
measured Mass Range: m/z = 403.5-405

TABLE 83B

| | | protein | | |
|---|---|---|---|---|
| FLJ26267 - Metergotamine | | concentration (uM) | | |
| (Metergoline) | | 0 | 9.5 | 19.0 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.4 | 0.2 | 0.8 |
| | 250 | 1.3 | 1.5 | 2.1 |

Minerals (+)
measured Mass Range: m/z = 403.5-405

TABLE 84A

| | | protein | | |
|---|---|---|---|---|
| | | concentration (uM) | | |
| FLJ26267 - Methoxamine | | 0 | 9.5 | 19.0 |
| compound | 0 | 0.0 | 0.0 | 0.0 |
| concentration | 1 | 0.2 | −0.2 | 0.3 |
| (uM) | 10 | 0.4 | 0.9 | 1.0 |
| | 100 | 7.7 | 7.0 | 9.5 |
| | 250 | 17.7 | 23.7 | 28.6 |

Minerals (−)
measured Mass Range: m/z = 211.3-212.8

TABLE 84B

| | | protein | | |
|---|---|---|---|---|
| | | concentration (uM) | | |
| FLJ26267 - Methoxamine | | 0 | 9.5 | 19.0 |
| compound | 0 | 0.0 | 0.0 | 0.0 |
| concentration | 1 | −0.5 | −0.2 | 0.0 |
| (uM) | 10 | 0.1 | 0.4 | 0.3 |
| | 100 | 5.7 | 6.4 | 5.9 |
| | 250 | 21.4 | 9.9 | 22.9 |

Minerals (+)
measured Mass Range: m/z = 211.3-212.8

TABLE 85A

| | | protein | | |
|---|---|---|---|---|
| | | concentration (uM) | | |
| FLJ26267 - Paroxetine | | 0 | 9.5 | 19.0 |
| compound | 0 | 0.0 | 0.0 | 0.0 |
| concentration | 1 | 0.0 | 0.1 | 0.0 |
| (uM) | 10 | 0.1 | 0.3 | 0.2 |
| | 100 | 1.7 | 2.7 | 1.7 |
| | 250 | 5.2 | 5.7 | 6.7 |

Minerals (−)
measured Mass Range: m/z = 329.4-330.9

TABLE 85B

| | | protein | | |
|---|---|---|---|---|
| | | concentration (uM) | | |
| FLJ26267 - Paroxetine | | 0 | 9.5 | 19.0 |
| compound | 0 | 0.0 | 0.0 | 0.0 |
| concentration | 1 | 0.0 | 0.1 | 0.0 |
| (uM) | 10 | 0.1 | 0.2 | 0.1 |
| | 100 | 1.6 | 2.7 | 2.2 |
| | 250 | 2.7 | 1.9 | 5.5 |

Minerals (+)
measured Mass Range: m/z = 329.4-330.9

TABLE 86A

| | | protein | | |
|---|---|---|---|---|
| | | concentration (uM) | | |
| FLJ26267 - Dizocilpine | | 0 | 9.5 | 19.0 |
| compound | 0 | 0.0 | 0.0 | 0.0 |
| concentration | 1 | 0.3 | 0.3 | 0.1 |
| (uM) | 10 | 0.8 | 1.3 | 0.8 |
| | 100 | 6.4 | 8.3 | 7.6 |
| | 250 | 14.2 | 17.2 | 16.5 |

Minerals (−)
measured Mass Range: m/z = 221.3-222.8

TABLE 86B

| | | protein | | |
|---|---|---|---|---|
| | | concentration (uM) | | |
| FLJ26267 - Dizocilpine | | 0 | 9.5 | 19.0 |
| compound | 0 | 0.0 | 0.0 | 0.0 |
| concentration | 1 | −0.6 | −0.3 | −0.1 |
| (uM) | 10 | 0.3 | 0.9 | 0.7 |
| | 100 | 8.7 | 8.4 | 8.3 |
| | 250 | 20.5 | 21.4 | 25.6 |

Minerals (+)
measured Mass Rangem/z= 221.3-222.8

TABLE 87A

| | | protein | | |
|---|---|---|---|---|
| | | concentration (uM) | | |
| FLJ26267 - Fluvoxamine | | 0 | 9.5 | 19.0 |
| compound | 0 | 0.0 | 0.0 | 0.0 |
| concentration | 1 | −0.3 | 0.0 | 0.1 |
| (uM) | 10 | −0.1 | 0.5 | 0.3 |
| | 100 | 4.0 | 9.4 | 9.1 |
| | 250 | 14.5 | 15.6 | 21.9 |

Minerals (−)
measured Mass Range: m/z = 318.3-319.8

TABLE 87B

| | | protein | | |
|---|---|---|---|---|
| | | concentration (uM) | | |
| FLJ26267 - Fluvoxamine | | 0 | 9.5 | 19.0 |
| compound | 0 | 0.0 | 0.0 | 0.0 |
| concentration | 1 | 0.0 | −0.4 | 0.0 |
| (uM) | 10 | 0.3 | 0.4 | 0.4 |
| | 100 | 5.5 | 7.6 | 8.1 |
| | 250 | 18.1 | 20.0 | 17.0 |

Minerals (+)
measured Mass Range: m/z = 318.3-319.8

TABLE 88A

| FLJ26267 - | | protein | | |
|---|---|---|---|---|
| 3-Hydroxykynurenine | | concentration (uM) | | |
| (3-Hydroxykynurenine (R,S)) | | 0 | 9.5 | 19.0 |
| compound | 0 | 0.0 | 0.0 | 0.0 |
| concentration | 1 | 0.2 | −0.1 | 0.0 |
| (uM) | 10 | 1.1 | 0.9 | −0.1 |
| | 100 | 1.8 | 4.1 | 3.8 |
| | 250 | 4.8 | 7.9 | 3.5 |

Minerals (−)
measured Mass Range: m/z = 224.2-225.7

TABLE 88B

| FLJ26267 - | | protein | | |
|---|---|---|---|---|
| 3-Hydroxykynurenine | | concentration (uM) | | |
| (3-Hydroxykynurenine (R,S)) | | 0 | 9.5 | 19.0 |
| compound | 0 | 0.0 | 0.0 | 0.0 |
| concentration | 1 | −1.5 | 2.5 | 2.5 |
| (uM) | 10 | 0.6 | 0.8 | 1.2 |
| | 100 | 2.5 | 5.6 | 3.6 |
| | 250 | 7.1 | 6.5 | 3.2 |

Minerals (+)
measured Mass Range: m/z = 224.2-225.7

TABLE 89A

| | | protein | | |
|---|---|---|---|---|
| FLJ26267 - | | concentration (uM) | | |
| Nimetazepam | | 0 | 9.5 | 19.0 |
| compound | 0 | 0.0 | 0.0 | 0.0 |
| concentration | 1 | 0.0 | 0.0 | 0.0 |
| (uM) | 10 | 0.2 | 0.2 | 0.2 |
| | 100 | 2.6 | 4.6 | 2.3 |
| | 250 | 1.9 | 10.8 | 11.3 |

Minerals (−)
measured Mass Range: m/z = 295.3-296.8

TABLE 89B

| | | protein | | |
|---|---|---|---|---|
| FLJ26267 - | | concentration (uM) | | |
| Nimetazepam | | 0 | 9.5 | 19.0 |
| compound | 0 | 0.0 | 0.0 | 0.0 |
| concentration | 1 | 0.0 | 0.0 | 0.0 |
| (uM) | 10 | 0.2 | 0.3 | 0.3 |
| | 100 | 6.2 | 5.5 | 4.8 |
| | 250 | 2.0 | 3.2 | 15.8 |

Minerals (+)
measured Mass Range: m/z = 295.3-296.8

TABLE 90A

| FLJ26267 - | | protein | | |
|---|---|---|---|---|
| Fludroxycortide | | concentration (uM) | | |
| (Flurandrenolide) | | 0 | 9.5 | 19.0 |
| compound | 0 | 0.0 | 0.0 | 0.0 |
| concentration | 1 | −0.6 | 0.7 | 0.3 |
| (uM) | 10 | 0.0 | 1.3 | 0.6 |
| | 100 | 5.9 | 8.3 | 7.1 |
| | 250 | 17.4 | 20.8 | 19.6 |

Minerals (−)
measured Mass Range: m/z = 436.5-438

TABLE 90B

| FLJ26267 - | | protein | | |
|---|---|---|---|---|
| Fludroxycortide | | concentration (uM) | | |
| (Flurandrenolide) | | 0 | 9.5 | 19.0 |
| compound | 0 | 0.0 | 0.0 | 0.0 |
| concentration | 1 | −0.2 | −0.1 | 0.2 |
| (uM) | 10 | 0.7 | 0.5 | 0.8 |
| | 100 | 7.7 | 10.2 | 10.0 |
| | 250 | 21.1 | 8.5 | 25.7 |

Minerals (+)
measured Mass Range: m/z = 436.5-438

TABLE 91A

| | | protein | | |
|---|---|---|---|---|
| | | concentration (uM) | | |
| FLJ26062 - Fenoprofen | | 0 | 23.8 | 47.5 |
| compound | 0 | 0.0 | 0.0 | 0.0 |
| concentration | 1 | 0.2 | −0.5 | −0.3 |
| (uM) | 10 | −0.6 | −0.6 | 0.3 |
| | 100 | 3.7 | 9.4 | 17.2 |
| | 250 | 22.1 | 32.4 | 32.8 |

Minerals (−)
measured Mass Range: m/z = 242.3-243.8

TABLE 91B

| | | protein | | |
|---|---|---|---|---|
| | | concentration (uM) | | |
| FLJ26062 - Fenoprofen | | 0 | 23.8 | 47.5 |
| compound | 0 | 0.0 | 0.0 | 0.0 |
| concentration | 1 | −2.4 | 0.2 | 0.4 |
| (uM) | 10 | −1.4 | 1.0 | 1.3 |
| | 100 | 4.2 | 12.7 | 17.6 |
| | 250 | 28.4 | 43.8 | 50.3 |

Minerals (+)
measured Mass Range: m/z = 242.3-243.8

TABLE 92A

| | | protein | | |
|---|---|---|---|---|
| FLJ22936 - | | concentration (uM) | | |
| Acenocoumarol | | 0 | 23.8 | 47.5 |
| compound | 0 | 0.0 | 0.0 | 0.0 |
| concentration | 1 | 0.0 | 0.1 | 0.1 |
| (uM) | 10 | 0.1 | 1.1 | 1.7 |
| | 100 | 1.6 | 23.6 | 31.8 |
| | 250 | 29.3 | 37.3 | 42.9 |

Minerals (−)
measured Mass Range: m/z = 353.3-354.8

TABLE 92B

| | | protein | | |
|---|---|---|---|---|
| FLJ22936 - | | concentration (uM) | | |
| Acenocoumarol | | 0 | 23.8 | 47.5 |
| compound | 0 | 0.0 | 0.0 | 0.0 |
| concentration | 1 | 0.0 | 0.0 | 0.0 |
| (uM) | 10 | 0.6 | 0.6 | 1.4 |
| | 100 | 12.7 | 21.1 | 23.3 |
| | 250 | 26.2 | 39.9 | 43.6 |

Minerals (+)
measured Mass Range: m/z = 353.3-354.8

TABLE 93A

| | | protein | | |
|---|---|---|---|---|
| | | concentration (uM) | | |
| FLJ22936 - Budesonide | | 0 | 23.8 | 47.5 |
| compound | 0 | 0.0 | 0.0 | 0.0 |
| concentration | 1 | −0.3 | −0.3 | −1.6 |
| (uM) | 10 | −0.7 | −0.5 | −0.6 |
| | 100 | 2.8 | 5.6 | 3.5 |
| | 250 | 4.0 | 6.4 | 6.0 |

Minerals (−)
measured Mass Range: m/z = 430.5-432

TABLE 93B

| | | protein | | |
|---|---|---|---|---|
| | | concentration (uM) | | |
| FLJ22936 - Budesonide | | 0 | 23.8 | 47.5 |
| compound | 0 | 0.0 | 0.0 | 0.0 |
| concentration | 1 | −2.7 | −2.2 | 1.6 |
| (uM) | 10 | −0.9 | 1.1 | 2.4 |
| | 100 | 5.0 | 8.0 | 8.3 |
| | 250 | 21.2 | 24.5 | 30.1 |

Minerals (+)
measured Mass Range: m/z = 430.5-432

TABLE 94A

| | | protein | | |
|---|---|---|---|---|
| FLJ22936 - | | concentration (uM) | | |
| Chlorogenic acid | | 0 | 23.8 | 47.5 |
| compound | 0 | 0.0 | 0.0 | 0.0 |
| concentration | 1 | −0.6 | −0.4 | 0.3 |
| (uM) | 10 | −0.2 | 0.3 | 0.7 |
| | 100 | 0.7 | 9.2 | 21.8 |
| | 250 | 10.8 | 22.5 | 24.1 |

Minerals (−)
measured Mass Range: m/z = 354.3-355.8

TABLE 94B

| | | protein | | |
|---|---|---|---|---|
| FLJ22936 - | | concentration (uM) | | |
| Chlorogenic acid | | 0 | 23.8 | 47.5 |
| compound | 0 | 0.0 | 0.0 | 0.0 |
| concentration | 1 | −2.0 | −1.0 | −0.8 |
| (uM) | 10 | −2.0 | −0.3 | 0.0 |
| | 100 | −0.6 | 1.4 | 2.3 |
| | 250 | 2.3 | 7.5 | 13.5 |

Minerals (+)
measured Mass Range: m/z = 354.3-355.8

TABLE 95A

| | | protein | | |
|---|---|---|---|---|
| FLJ22936 - | | concentration (uM) | | |
| Chlorothiazide | | 0 | 23.8 | 47.5 |
| compound | 0 | 0.0 | 0.0 | 0.0 |
| concentration | 1 | −0.1 | 0.0 | 0.0 |
| (uM) | 10 | 0.1 | 0.7 | 0.9 |
| | 100 | 2.9 | 5.2 | 3.9 |
| | 250 | 6.6 | 8.3 | 21.0 |

Minerals (−)
measured Mass Range: m/z = 295.7-297.2

TABLE 95B

| | | protein | | |
|---|---|---|---|---|
| | | concentration (uM) | | |
| FLJ22936 - Chlorothiazide | | 0 | 23.8 | 47.5 |
| compound | 0 | 0.0 | 0.0 | 0.0 |
| concentration | 1 | −0.1 | 0.0 | 0.0 |
| (uM) | 10 | 0.0 | 0.2 | −0.2 |
| | 100 | 1.6 | 4.1 | 5.4 |
| | 250 | 7.8 | 13.7 | 16.2 |

Minerals (+)
measured Mass Range: m/z = 295.7-297.2

TABLE 96A

| | | protein | | |
|---|---|---|---|---|
| | | concentration (uM) | | |
| FLJ22936 - Diclofenac | | 0 | 23.8 | 47.5 |
| compound | 0 | 0.0 | 0.0 | 0.0 |
| concentration | 1 | −0.2 | −0.2 | 0.1 |
| (uM) | 10 | −0.1 | 0.4 | 0.4 |
| | 100 | 0.6 | 5.3 | 8.6 |
| | 250 | 0.6 | 11.7 | 18.8 |

Minerals (−)
measured Mass Range: m/z = 296.2-297.7

TABLE 96B

| | | protein | | |
|---|---|---|---|---|
| | | concentration (uM) | | |
| FLJ22936 - Diclofenac | | 0 | 23.8 | 47.5 |
| compound | 0 | 0.0 | 0.0 | 0.0 |
| concentration | 1 | −0.4 | −0.1 | 0.0 |
| (uM) | 10 | −0.6 | 0.2 | 0.1 |
| | 100 | −0.2 | 2.5 | 2.8 |
| | 250 | 1.9 | 5.8 | 8.5 |

Minerals (+)
measured Mass Range: m/z = 296.2-297.7

TABLE 97A

| | | protein | | |
|---|---|---|---|---|
| | | concentration (uM) | | |
| FLJ22936 - Diperodon | | 0 | 23.8 | 47.5 |
| compound | 0 | 0.0 | 0.0 | 0.0 |
| concentration | 1 | 0.0 | 0.0 | 0.0 |
| (uM) | 10 | 0.3 | 0.6 | 0.5 |
| | 100 | 4.1 | 6.2 | 7.7 |
| | 250 | 8.5 | 8.6 | 11.0 |

Minerals (−)
measured Mass Range: m/z = 397.5-399

TABLE 97B

| | | protein | | |
|---|---|---|---|---|
| | | concentration (uM) | | |
| FLJ22936 - Diperodon | | 0 | 23.8 | 47.5 |
| compound | 0 | 0.0 | 0.0 | 0.0 |
| concentration | 1 | 0.0 | 0.1 | 0.0 |
| (uM) | 10 | 0.2 | 0.6 | 0.9 |
| | 100 | 5.0 | 8.4 | 8.3 |
| | 250 | 13.1 | 27.2 | 28.8 |

Minerals (+)
measured Mass Range: m/z = 397.5-399

TABLE 98A

| | | protein | | |
|---|---|---|---|---|
| | | concentration (uM) | | |
| FLJ22936 - DO 897/99 | | 0 | 23.8 | 47.5 |
| compound | 0 | 0.0 | 0.0 | 0.0 |
| concentration | 1 | 0.0 | 0.1 | 0.0 |
| (uM) | 10 | 0.1 | 0.7 | 1.0 |
| | 100 | 2.5 | 5.3 | 5.7 |
| | 250 | 2.6 | 6.3 | 11.1 |

Minerals (−)
measured Mass Range: m/z = 417.6-419.1

TABLE 98B

| | | protein | | |
|---|---|---|---|---|
| | | concentration (uM) | | |
| FLJ22936 - DO 897/99 | | 0 | 23.8 | 47.5 |
| compound | 0 | 0.0 | 0.0 | 0.0 |
| concentration | 1 | −0.1 | 0.1 | 0.0 |
| (uM) | 10 | 0.0 | 0.5 | 0.7 |
| | 100 | 1.6 | 7.5 | 7.5 |
| | 250 | 5.7 | 14.7 | 18.0 |

Minerals (+)
measured Mass Range: m/z = 417.6-419.1

TABLE 99A

| | | protein | | |
|---|---|---|---|---|
| | | concentration (uM) | | |
| FLJ22936 - Nimesulide | | 0 | 23.8 | 47.5 |
| compound | 0 | 0.0 | 0.0 | 0.0 |
| concentration | 1 | 0.0 | −0.1 | 0.0 |
| (uM) | 10 | 0.0 | −0.1 | 0.0 |
| | 100 | 0.3 | 0.9 | 1.4 |
| | 250 | 0.5 | 2.8 | 4.6 |

Minerals (−)
measured Mass Range: m/z = 308.3-309.8

TABLE 99B

| | | protein | | |
|---|---|---|---|---|
| | | concentration (uM) | | |
| FLJ22936 - Nimesulide | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 0.5 | 0.8 | 0.9 |
| | 250 | 1.5 | 1.9 | 3.2 |

Minerals (+)
measured Mass Range: m/z = 308.3-309.8

TABLE 100A

| | | protein | | |
|---|---|---|---|---|
| FLJ22936 - | | concentration (uM) | | |
| Thioproperasine | | 0 | 23.8 | 47.5 |
| compound | 0 | 0.0 | 0.0 | 0.0 |
| concentration | 1 | −0.1 | 0.0 | 0.0 |
| (uM) | 10 | 0.1 | 0.3 | 0.4 |
| | 100 | 2.4 | 3.7 | 5.0 |
| | 250 | 5.2 | 4.1 | 12.1 |

Minerals (−)
measured Mass Range: m/z = 446.8-448.3

TABLE 100B

| | | protein | | |
|---|---|---|---|---|
| FLJ22936 - | | concentration (uM) | | |
| Thioproperasine | | 0 | 23.8 | 47.5 |
| compound | 0 | 0.0 | 0.0 | 0.0 |
| concentration | 1 | −2.2 | −0.2 | −0.1 |
| (uM) | 10 | −2.2 | −0.1 | 0.1 |
| | 100 | −0.4 | 1.3 | 2.3 |
| | 250 | 3.5 | 6.4 | 11.4 |

Minerals (+)
measured Mass Range: m/z = 446.8-448.3

TABLE 101A

| | | protein | | |
|---|---|---|---|---|
| | | concentration (uM) | | |
| FLJ22936 - Sarpogrelate | | 0 | 23.8 | 47.5 |
| compound | 0 | 0.0 | 0.0 | 0.0 |
| concentration | 1 | 0.0 | 0.4 | 0.2 |
| (uM) | 10 | 0.2 | 1.4 | 1.3 |
| | 100 | 5.0 | 9.9 | 10.0 |
| | 250 | 8.2 | 14.2 | 13.9 |

Minerals (−)
measured Mass Range: m/z = 429.5-431

TABLE 101B

| | | protein | | |
|---|---|---|---|---|
| | | concentration (uM) | | |
| FLJ22936 - Sarpogrelate | | 0 | 23.8 | 47.5 |
| compound | 0 | 0.0 | 0.0 | 0.0 |
| concentration | 1 | 0.1 | 0.0 | 0.0 |
| (uM) | 10 | 0.4 | 1.1 | 1.2 |
| | 100 | 6.1 | 8.6 | 10.8 |
| | 250 | 13.2 | 24.2 | 27.4 |

Minerals (+)
measured Mass Range: m/z = 429.5-431

TABLE 102A

| | | protein | | |
|---|---|---|---|---|
| FLJ43223 - | | concentration (uM) | | |
| Acetylsalicylsalicylic acid | | 0 | 23.8 | 47.5 |
| compound | 0 | 0.0 | 0.0 | 0.0 |
| concentration | 1 | −0.1 | 0.3 | 0.0 |
| (uM) | 10 | 1.0 | 1.5 | 1.4 |
| | 100 | 8.9 | 12.3 | 11.8 |
| | 250 | 28.7 | 32.1 | 32.0 |

Minerals (−)
measured Mass Range: m/z = 300.3-301.8

TABLE 102B

| | | protein | | |
|---|---|---|---|---|
| FLJ43223 - | | concentration (uM) | | |
| Acetylsalicylsalicylic acid | | 0 | 23.8 | 47.5 |
| compound | 0 | 0.0 | 0.0 | 0.0 |
| concentration | 1 | −0.1 | 0.1 | 0.0 |
| (uM) | 10 | 0.8 | 1.2 | 1.3 |
| | 100 | 10.6 | 9.9 | 16.3 |
| | 250 | 41.5 | 35.1 | 38.3 |

Minerals (+)
measured Mass Range: m/z = 300.3-301.8

TABLE 103A

| | | protein | | |
|---|---|---|---|---|
| FLJ26102 - | | concentration (uM) | | |
| Buspirone | | 0 | 23.8 | 47.5 |
| compound | 0 | 0.0 | 0.0 | 0.0 |
| concentration | 1 | 0.0 | 0.1 | 0.1 |
| (uM) | 10 | 1.2 | 2.3 | 2.8 |
| | 100 | 9.7 | 18.8 | 18.6 |
| | 250 | 34.0 | 29.7 | 39.7 |

Minerals (−)
measured Mass Range: m/z = 385.5-387

TABLE 103B

| | | protein | | |
|---|---|---|---|---|
| FLJ26102 - | | concentration (uM) | | |
| Buspirone | | 0 | 23.8 | 47.5 |
| compound | 0 | 0.0 | 0.0 | 0.0 |
| concentration | 1 | 0.0 | 0.1 | 0.1 |
| (uM) | 10 | 0.7 | 1.5 | 2.0 |
| | 100 | 10.1 | 18.9 | 9.2 |
| | 250 | 17.2 | 19.4 | 41.0 |

Minerals (+)
measured Mass Range: m/z = 385.5-387

TABLE 104A

| | | protein | | |
|---|---|---|---|---|
| FLJ25218 - | | concentration (uM) | | |
| Dopamine | | 0 | 23.8 | 47.5 |
| compound | 0 | 0.0 | 0.0 | 0.0 |
| concentration | 1 | 1.0 | 0.9 | 1.3 |
| (uM) | 10 | 6.1 | 3.1 | 2.0 |
| | 100 | 6.2 | 7.0 | 4.6 |
| | 250 | 12.7 | 15.8 | 20.5 |

Minerals (−)
measured Mass Range: m/z = 153.2-154.7

TABLE 104B

| | | protein | | |
|---|---|---|---|---|
| FLJ25218 - | | concentration (uM) | | |
| Dopamine | | 0 | 23.8 | 47.5 |
| compound | 0 | 0.0 | 0.0 | 0.0 |
| concentration | 1 | 23.6 | 9.6 | −11.4 |
| (uM) | 10 | 20.0 | 7.6 | −8.0 |
| | 100 | 14.3 | 18.0 | −1.7 |
| | 250 | 30.0 | 45.1 | 16.7 |

Minerals (+)
measured Mass Range: m/z = 153.2-154.7

TABLE 105

| | | protein | | |
|---|---|---|---|---|
| FLJ25218 - | | concentration | | |
| Alpha-methyl-5- | | (uM) | | |
| hydroxytryptamine | | 0 | 23.8 | 47.5 |
| compound | 0 | 0.0 | 0.0 | 0.0 |
| concentration | 1 | 3.4 | −0.6 | 0.7 |
| (uM) | 10 | 1.9 | 0.4 | 1.8 |
| | 100 | 1.9 | 2.7 | 1.7 |
| | 250 | 6.5 | 4.7 | 9.7 |

Minerals (+)
measured Mass Rangem/z = 190.2-191.7

TABLE 106A

| | | protein | | |
|---|---|---|---|---|
| FLJ45675 - | | concentration (uM) | | |
| Cisapride | | 0 | 23.8 | 47.5 |
| compound | 0 | 0.0 | 0.0 | 0.0 |
| concentration | 1 | −0.2 | 0.2 | 0.2 |
| (uM) | 10 | 0.0 | 0.2 | 0.2 |
| | 100 | 0.2 | 1.9 | 3.2 |
| | 250 | 0.1 | 6.6 | 7.2 |

Minerals (−)
measured Mass Range: m/z = 466-467.5

TABLE 106B

| | | protein | | |
|---|---|---|---|---|
| | | concentration | | |
| FLJ45675 - | | (uM) | | |
| Cisapride | | 0 | 23.8 | 47.5 |
| compound | 0 | 0.0 | 0.0 | 0.0 |
| concentration | 1 | 0.2 | 0.0 | −0.5 |
| (uM) | 10 | 0.2 | 0.1 | −0.2 |
| | 100 | 0.5 | 2.5 | 3.2 |
| | 250 | 0.3 | 9.2 | 8.1 |

Minerals (+)
measured Mass Range: m/z = 466-467.5

TABLE 107

| | | protein | | |
|---|---|---|---|---|
| | | concentration | | |
| FLJ25918 - | | (uM) | | |
| Berberine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.2 | 0.3 |
| | 100 | 0.6 | 2.2 | 7.4 |
| | 250 | 0.8 | 2.5 | 9.2 |

Minerals (+)
measured Mass Range: m/z = 336.3-337.8

TABLE 108A

| | | protein | | |
|---|---|---|---|---|
| | | concentration | | |
| FLJ25918 - | | (uM) | | |
| Celestin blue | | 0 | 23.8 | 47.5 |
| compound | 0 | 0.0 | 0.0 | 0.0 |
| concentration | 1 | 0.0 | 0.1 | 0.1 |
| (uM) | 10 | 0.0 | 4.6 | 7.2 |
| | 100 | 0.0 | 9.1 | 24.3 |
| | 250 | 0.0 | 19.7 | 44.5 |

Minerals (−)
measured Mass Range: m/z = 328.4-329.9

TABLE 108B

| | | protein | | |
|---|---|---|---|---|
| | | concentration | | |
| FLJ25918 - | | (uM) | | |
| Celestin blue | | 0 | 23.8 | 47.5 |
| compound | 0 | 0.0 | 0.0 | 0.0 |
| concentration | 1 | 0.0 | 0.1 | 0.1 |
| (uM) | 10 | 0.0 | 1.8 | 4.3 |
| | 100 | 0.2 | 8.6 | 19.0 |
| | 250 | 0.3 | 8.2 | 38.5 |

Minerals (+)
measured Mass Range: m/z = 328.4-329.9

TABLE 109

| | | protein | | |
|---|---|---|---|---|
| | | concentration | | |
| FLJ25918 - | | (uM) | | |
| Diflunisal | | 0 | 23.8 | 47.5 |
| compound | 0 | 0.0 | 0.0 | 0.0 |
| concentration | 1 | 0.1 | 0.1 | 0.3 |
| (uM) | 10 | 0.1 | 0.8 | 1.8 |
| | 100 | 0.1 | 1.5 | 3.0 |
| | 250 | 1.0 | 2.9 | 7.9 |

Minerals (+)
measured Mass Range: m/z = 250.2-251.7

TABLE 110A

| | | protein | | |
|---|---|---|---|---|
| | | concentration | | |
| FLJ25918 - | | (uM) | | |
| Mebendazole | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.4 |
| | 100 | 0.0 | 1.1 | 0.0 |
| | 250 | 0.0 | 2.6 | 3.7 |

Minerals (−)
measured Mass Range: m/z = 295.3-296.8

TABLE 110B

| | | protein | | |
|---|---|---|---|---|
| | | concentration | | |
| FLJ25918 - | | (uM) | | |
| Mebendazole | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 0.0 | 0.7 | 1.4 |
| | 250 | 0.0 | 0.7 | 0.5 |

Minerals (+)
measured Mass Range: m/z = 295.3-296.8

TABLE 111A

| | | protein | | |
|---|---|---|---|---|
| | | concentration | | |
| FLJ25918 - | | (uM) | | |
| Tranilast | | 0 | 23.8 | 47.5 |
| compound | 0 | 0.0 | 0.0 | 0.0 |
| concentration | 1 | 0.0 | 0.3 | 0.3 |
| (uM) | 10 | 0.0 | 3.3 | 6.6 |
| | 100 | 0.0 | 5.9 | 15.5 |
| | 250 | 1.1 | 14.9 | 19.9 |

Minerals (−)
measured Mass Range: m/z = 327.3-328.8

TABLE 111B

| | | protein | | |
|---|---|---|---|---|
| | | concentration | | |
| FLJ25918 - | | (uM) | | |
| Tranilast | | 0 | 23.8 | 47.5 |
| compound | 0 | 0.0 | 0.0 | 0.0 |
| concentration | 1 | 0.0 | 0.1 | 0.1 |
| (uM) | 10 | 0.0 | 3.1 | 7.1 |
| | 100 | 0.2 | 7.6 | 6.7 |
| | 250 | 1.8 | 9.1 | 17.6 |

Minerals (+)
measured Mass Range: m/z = 327.3-328.8

TABLE 112A

| | | protein | | |
|---|---|---|---|---|
| | | concentration | | |
| FLJ46709 - | | (uM) | | |
| Bromperidol | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 1.0 | 2.7 | 3.4 |
| | 250 | 3.3 | 9.2 | 8.5 |

Minerals (−)
measured Mass Range: m/z = 420.3-421.8

TABLE 112B

| | | protein | | |
|---|---|---|---|---|
| | | concentration | | |
| FLJ46709 - | | (uM) | | |
| Bromperidol | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.2 |
| | 100 | 1.2 | 3.0 | 4.3 |
| | 250 | 4.8 | 10.5 | 22.4 |

Minerals (+)
measured Mass Range: m/z = 420.3-421.8

TABLE 113A

| | | protein | | |
|---|---|---|---|---|
| | | concentration | | |
| FLJ46709 - | | (uM) | | |
| Coralyne | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 0.1 | 1.2 | 0.5 |
| | 250 | 0.2 | 3.1 | 3.1 |

Minerals (−)
measured Mass Range: m/z = 364.4-365.9

TABLE 113B

| | | protein | | |
|---|---|---|---|---|
| FLJ46709 - | | concentration (uM) | | |
| Coralyne | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.0 | 0.2 |
| | 100 | 0.2 | 1.2 | 1.6 |
| | 250 | 0.9 | 9.8 | 7.1 |

Minerals (+)
measured Mass Range: m/z = 364.4-365.9

TABLE 114

| | | protein | | |
|---|---|---|---|---|
| | | concentration | | |
| RGNpc017 - | | (uM) | | |
| DO 897/99 | | 0 | 23.8 | 47.5 |
| compound | 0 | 0.0 | 0.0 | 0.0 |
| concentration | 1 | 0.0 | 0.0 | 0.0 |
| (uM) | 10 | 0.1 | 0.3 | 0.2 |
| | 100 | 3.1 | 2.7 | 2.1 |
| | 250 | 6.9 | 7.4 | 13.2 |

Minerals (+)
measured Mass Range: m/z = 417.6-419.1

TABLE 115A

| | | protein | | |
|---|---|---|---|---|
| | | concentration | | |
| RGNpc017 - | | (uM) | | |
| Domperidone | | 0 | 23.8 | 47.5 |
| compound | 0 | 0.0 | 0.0 | 0.0 |
| concentration | 1 | 0.0 | 0.0 | 0.0 |
| (uM) | 10 | 0.1 | 0.1 | 0.0 |
| | 100 | 0.8 | 1.5 | 2.0 |
| | 250 | 2.6 | 3.4 | 5.1 |

Minerals (−)
measured Mass Range: m/z = 425.9-427.4

TABLE 115B

| | | protein | | |
|---|---|---|---|---|
| RGNpc017 - | | concentration (uM) | | |
| Domperidone | | 0 | 23.8 | 47.5 |
| compound | 0 | 0.0 | 0.0 | 0.0 |
| concentration | 1 | −0.2 | 0.0 | 0.1 |
| (uM) | 10 | −0.1 | 0.1 | 0.2 |
| | 100 | 0.9 | 2.5 | 5.0 |
| | 250 | 1.4 | 4.2 | 4.5 |

Minerals (+)
measured Mass Range: m/z = 425.9-427.4

TABLE 116

| RGNpc017 - | | protein | | |
|---|---|---|---|---|
| Flupentixol | | concentration (uM) | | |
| (Flupentixol (Z)) | | 0 | 23.8 | 47.5 |
| compound | 0 | 0.0 | 0.0 | 0.0 |
| concentration | 1 | −0.1 | 0.0 | 0.1 |
| (uM) | 10 | −0.1 | 0.1 | 0.3 |
| | 100 | 0.1 | 3.9 | 5.2 |
| | 250 | 1.7 | 13.2 | 25.5 |

Minerals (+)
measured Mass Range: m/z = 434.5-436

TABLE 117A

| | | protein | | |
|---|---|---|---|---|
| | | concentration | | |
| RGNpc017 - | | (uM) | | |
| Fluphenazine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.2 | 0.2 |
| | 100 | 0.8 | 3.3 | 3.8 |
| | 250 | 2.3 | 10.7 | 12.4 |

Minerals (−)
measured Mass Range: m/z = 324.4-325.9

TABLE 117B

| | | protein | | |
|---|---|---|---|---|
| RGNpc017 - | | concentration (uM) | | |
| Fluphenazine | | 0 | 23.8 | 47.5 |
| compound | 0 | 0.0 | 0.0 | 0.0 |
| concentration | 1 | −0.1 | 0.1 | 0.0 |
| (uM) | 10 | 0.0 | 0.2 | 0.5 |
| | 100 | 1.3 | 12.9 | 15.7 |
| | 250 | 4.1 | 29.0 | 44.6 |

Minerals (+)
measured Mass Range: m/z = 324.4-325.9

TABLE 118

| | | protein | | |
|---|---|---|---|---|
| | | concentration | | |
| RGNpc017 - | | (uM) | | |
| Thyroxine L | | 0 | 23.8 | 47.5 |
| compound | 0 | 0.0 | 0.0 | 0.0 |
| concentration | 1 | 0.5 | 0.3 | −0.1 |
| (uM) | 10 | 0.4 | 1.9 | 2.4 |
| | 100 | 0.4 | 9.7 | 18.3 |
| | 250 | 0.2 | 9.0 | 16.6 |

Minerals (+)
measured Mass Range: m/z = 776.9-778.4

TABLE 119A

| | | protein | | |
|---|---|---|---|---|
| | | concentration | | |
| RGNpc017 - | | (uM) | | |
| Trifluoperazine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.8 | 2.8 | 1.9 |
| | 250 | 3.7 | 14.0 | 14.2 |

Minerals (−)
measured Mass Range: m/z = 407.5-409

TABLE 119B

| | | protein | | |
|---|---|---|---|---|
| | | concentration | | |
| RGNpc017 - | | (uM) | | |
| Trifluoperazine | | 0 | 23.8 | 47.5 |
| compound | 0 | 0.0 | 0.0 | 0.0 |
| concentration | 1 | 0.0 | 0.0 | 0.1 |
| (uM) | 10 | 0.0 | 0.3 | 0.6 |
| | 100 | 0.8 | 10.8 | 15.8 |
| | 250 | 2.1 | 24.1 | 52.5 |

Minerals (+)
measured Mass Range: m/z = 407.5-409

TABLE 120A

| | | protein | | |
|---|---|---|---|---|
| RGNpc017 - | | concentration (uM) | | |
| Clinofibrate | | 0 | 23.8 | 47.5 |
| compound | 0 | 0.0 | 0.0 | 0.0 |
| concentration | 1 | 0.1 | 0.1 | 0.2 |
| (uM) | 10 | −0.1 | 0.4 | 0.9 |
| | 100 | 5.3 | 12.6 | 13.2 |
| | 250 | 3.1 | 18.9 | 19.5 |

Minerals (−)
measured Mass Range: m/z = 468.6-470.1

TABLE 120B

| | | protein | | |
|---|---|---|---|---|
| RGNpc017 - | | concentration (uM) | | |
| Clinofibrate | | 0 | 23.8 | 47.5 |
| compound | 0 | 0.0 | 0.0 | 0.0 |
| concentration | 1 | 0.1 | 0.4 | 0.7 |
| (uM) | 10 | −0.1 | 0.2 | 0.5 |
| | 100 | 10.2 | 12.4 | 15.9 |
| | 250 | 4.9 | 16.4 | 28.8 |

Minerals (+)
measured Mass Range: m/z = 468.6-470.1

TABLE 121A

| | | protein | | |
|---|---|---|---|---|
| RGNpc017 - | | concentration (uM) | | |
| Acetohexamide | | 0 | 23.8 | 47.5 |
| compound | 0 | 0.0 | 0.0 | 0.0 |
| concentration | 1 | 0.1 | 0.0 | 0.0 |
| (uM) | 10 | 0.5 | 0.4 | 0.4 |
| | 100 | 3.7 | 4.4 | 7.3 |
| | 250 | 11.5 | 10.3 | 5.7 |

Minerals (−)
measured Mass Range: m/z = 324.4-325.9

TABLE 121B

| | | protein | | |
|---|---|---|---|---|
| RGNpc017 - | | concentration (uM) | | |
| Acetohexamide | | 0 | 23.8 | 47.5 |
| compound | 0 | 0.0 | 0.0 | 0.0 |
| concentration | 1 | 0.0 | 0.1 | −0.1 |
| (uM) | 10 | 0.2 | 0.6 | 0.5 |
| | 100 | 6.1 | 4.3 | 4.4 |
| | 250 | 9.9 | 10.4 | 13.1 |

Minerals (+)
measured Mass Range: m/z = 324.4-325.9

TABLE 122A

| | | protein | | |
|---|---|---|---|---|
| RGNpc017 - | | concentration (uM) | | |
| Chromomycin A3 | | 0 | 23.8 | 47.5 |
| compound | 0 | 0.0 | 0.0 | 0.0 |
| concentration | 1 | 0.0 | 0.0 | 0.0 |
| (uM) | 10 | 0.1 | 0.1 | 0.7 |
| | 100 | 15.2 | 19.6 | 16.9 |
| | 250 | 107.1 | 127.4 | 146.7 |

Minerals (−)
measured Mass Range: m/z = 1183.3-1184.8

TABLE 122B

| | | protein | | |
|---|---|---|---|---|
| RGNpc017 - | | concentration (uM) | | |
| Chromomycin A3 | | 0 | 23.8 | 47.5 |
| compound | 0 | 0.0 | 0.0 | 0.0 |
| concentration | 1 | −0.1 | 0.1 | 0.1 |
| (uM) | 10 | −0.2 | 0.2 | 0.6 |
| | 100 | 14.5 | 13.4 | 9.7 |
| | 250 | 137.9 | 134.3 | 119.8 |

Minerals (+)
measured Mass Range: m/z = 1183.3-1184.8

TABLE 123A

| | | protein | | |
|---|---|---|---|---|
| RGNpc017 - | | concentration (uM) | | |
| Carboprost | | 0 | 23.8 | 47.5 |
| compound | 0 | 0.0 | 0.0 | 0.0 |
| concentration | 1 | −0.1 | 0.4 | 0.0 |
| (uM) | 10 | 1.0 | 1.1 | 2.1 |
| | 100 | 21.6 | 16.3 | 21.3 |
| | 250 | 50.9 | 54.4 | 65.0 |

Minerals (−)
measured Mass Range: m/z = 368.5-370

TABLE 123B

| | | protein | | |
|---|---|---|---|---|
| RGNpc017 - | | concentration (uM) | | |
| Carboprost | | 0 | 23.8 | 47.5 |
| compound | 0 | 0.0 | 0.0 | 0.0 |
| concentration | 1 | 0.0 | 0.2 | 0.6 |
| (uM) | 10 | 0.5 | 1.3 | 2.4 |
| | 100 | 17.3 | 18.8 | 21.4 |
| | 250 | 52.9 | 51.6 | 48.4 |

Minerals (+)
measured Mass Range: m/z = 368.5-370

TABLE 124

| | | protein | | |
|---|---|---|---|---|
| | | concentration (uM) | | |
| FLJ40377 - Alfuzocin | | 0 | 23.8 | 47.5 |
| compound | 0 | 0.0 | 0.0 | 0.0 |
| concentration | 1 | 0.0 | 0.1 | 0.1 |
| (uM) | 10 | 0.9 | 1.8 | 2.0 |
| | 100 | 7.8 | 13.3 | 16.0 |
| | 250 | 28.6 | 32.2 | 39.0 |

Minerals (+)
measured Mass Range: m/z = 389-390.5

TABLE 125A

| | | protein | | |
|---|---|---|---|---|
| FLJ40377 - Clobetasone | | concentration (uM) | | |
| (Clobetasone butyrate) | | 0 | 23.8 | 47.5 |
| compound | 0 | 0.0 | 0.0 | 0.0 |
| concentration | 1 | −0.2 | 0.0 | 0.1 |
| (uM) | 10 | 0.0 | 0.1 | 0.2 |
| | 100 | 0.0 | 3.8 | 0.6 |
| | 250 | 0.0 | 1.1 | 0.3 |

Minerals (−)
measured Mass Range: m/z = 479-480.5

TABLE 125B

| | | protein | | |
|---|---|---|---|---|
| FLJ40377 - Clobetasone | | concentration (uM) | | |
| (Clobetasone butyrate) | | 0 | 23.8 | 47.5 |
| compound | 0 | 0.0 | 0.0 | 0.0 |
| concentration | 1 | 0.6 | 0.7 | −1.1 |
| (uM) | 10 | 1.8 | 1.9 | 0.0 |
| | 100 | −4.7 | −1.4 | 10.4 |
| | 250 | 1.5 | 13.1 | 134.7 |

Minerals (+)
measured Mass Range: m/z = 479-480.5

TABLE 126A

| | | protein | | |
|---|---|---|---|---|
| | | concentration (uM) | | |
| FLJ40377 - Doxazosin | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 0.3 | 1.3 | 1.0 |
| | 250 | 0.2 | 1.4 | 2.4 |

Minerals (−)
measured Mass Range: m/z = 451.5-453

TABLE 126B

| | | protein | | |
|---|---|---|---|---|
| | | concentration (uM) | | |
| FLJ40377 - Doxazosin | | 0 | 23.8 | 47.5 |
| compound | 0 | 0.0 | 0.0 | 0.0 |
| concentration | 1 | −0.4 | −0.1 | 0.0 |
| (uM) | 10 | −0.4 | 0.0 | 0.2 |
| | 100 | −0.1 | 1.3 | 1.9 |
| | 250 | 0.0 | 4.5 | 7.5 |

Minerals (+)
measured Mass Range: m/z = 451.5-453

TABLE 127A

| | | protein | | |
|---|---|---|---|---|
| | | concentration (uM) | | |
| FLJ40377 - Pranlukast | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.2 |
| | 100 | 0.0 | 1.1 | 4.2 |
| | 250 | 0.0 | 0.9 | 2.6 |

Minerals (−)
measured Mass Range: m/z = 481.5-483

TABLE 127B

| | | protein | | |
|---|---|---|---|---|
| | | concentration (uM) | | |
| FLJ40377 - Pranlukast | | 0 | 23.8 | 47.5 |
| compound | 0 | 0.0 | 0.0 | 0.0 |
| concentration | 1 | −0.2 | 0.0 | 0.0 |
| (uM) | 10 | −0.2 | 0.0 | 0.1 |
| | 100 | −0.2 | 1.5 | 17.3 |
| | 250 | −0.1 | 0.2 | 5.1 |

Minerals (+)
measured Mass Range: m/z = 481.5-483

TABLE 128

| | | protein | | |
|---|---|---|---|---|
| | | concentration (uM) | | |
| FLJ40377 - Risperidone | | 0 | 23.8 | 47.5 |
| compound | 0 | 0.0 | 0.0 | 0.0 |
| concentration | 1 | 0.1 | 0.1 | 0.1 |
| (uM) | 10 | 0.9 | 2.4 | 3.6 |
| | 100 | 12.2 | 20.8 | 22.6 |
| | 250 | 18.5 | 40.9 | 34.9 |

Minerals (+)
measured Mass Range: m/z = 410.5-412

TABLE 129

| | | protein | | |
|---|---|---|---|---|
| FLJ25845 - | | concentration (uM) | | |
| Acetopromazine | | 0 | 11.9 | 23.8 |
| compound | 0 | 0.0 | 0.0 | 0.0 |
| concentration | 1 | 0.0 | −0.2 | 0.1 |
| (uM) | 10 | −0.1 | 0.0 | 0.2 |
| | 100 | 1.1 | 0.7 | 2.4 |
| | 250 | 3.0 | 5.4 | 8.6 |

Minerals (+)
measured Mass Range: m/z = 326.5-328

TABLE 130B

| | | protein concentration | | |
|---|---|---|---|---|
| FLJ25845 - | | (uM) | | |
| Cyclopentolate | | 0 | 11.9 | 23.8 |
| compound | 0 | 0.0 | 0.0 | 0.0 |
| concentration | 1 | −0.1 | −0.3 | 0.2 |
| (uM) | 10 | 1.1 | 0.2 | 0.6 |
| | 100 | 6.7 | 20.5 | 15.7 |
| | 250 | 35.1 | 40.2 | 51.1 |

Minerals (+)
measured Mass Range: m/z = 291.4-292.9

TABLE 131

| | | protein concentration | | |
|---|---|---|---|---|
| FLJ25845 - | | (uM) | | |
| Perhexiline | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.0 |
| | 100 | 1.3 | 1.9 | 0.8 |
| | 250 | 3.2 | 4.2 | 7.0 |

Minerals (+)
measured Mass Range: m/z = 277.6-279.1

TABLE 132

| | | protein concentration | | |
|---|---|---|---|---|
| FLJ25845 - | | (uM) | | |
| Phenformin | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.2 | 0.2 |
| | 100 | 2.1 | 3.8 | 3.5 |
| | 250 | 7.0 | 9.5 | 13.1 |

Minerals (+)
measured Mass Range: m/z = 205.3-206.8

TABLE 133

| | | protein concentration | | |
|---|---|---|---|---|
| FLJ25845 - | | (uM) | | |
| Pyrilamine | | 0 | 11.9 | 23.8 |
| compound | 0 | 0.0 | 0.0 | 0.0 |
| concentration | 1 | 0.1 | 0.1 | 0.0 |
| (uM) | 10 | 0.3 | 0.5 | 0.6 |
| | 100 | 4.6 | 15.2 | 13.8 |
| | 250 | 18.3 | 25.4 | 29.6 |

Minerals (+)
measured Mass Range: m/z = 285.5-287

TABLE 134

| | | protein concentration | | |
|---|---|---|---|---|
| FLJ25845 - | | (uM) | | |
| Terconazole | | 0 | 11.9 | 23.8 |
| compound | 0 | 0.0 | 0.0 | 0.0 |
| concentration | 1 | 0.0 | −0.2 | 0.0 |
| (uM) | 10 | 0.3 | 0.4 | 0.3 |
| | 100 | 2.1 | 5.6 | 7.0 |
| | 250 | 8.2 | 11.3 | 14.7 |

Minerals (+)
measured Mass Range: m/z = 532.5-534

TABLE 135

| | | protein concentration | | |
|---|---|---|---|---|
| FLJ25845 - | | (uM) | | |
| Tobramycin | | 0 | 23.8 | 47.5 |
| compound | 0 | 0.0 | 0.0 | 0.0 |
| concentration | 1 | −0.3 | −1.7 | 2.0 |
| (uM) | 10 | 2.6 | 1.2 | 1.6 |
| | 100 | 4.8 | 7.0 | 17.5 |
| | 250 | 16.3 | 11.2 | 28.7 |

Minerals (+)
measured Mass Range: m/z = 467.5-469

TABLE 136

| | | protein concentration | | |
|---|---|---|---|---|
| FLJ25845 - | | (uM) | | |
| Amoxapine | | 0 | 11.9 | 23.8 |
| compound | 0 | 0.0 | 0.0 | 0.0 |
| concentration | 1 | 0.0 | 0.0 | 0.0 |
| (uM) | 10 | 0.1 | 0.3 | 0.2 |
| | 100 | 2.8 | 2.7 | 3.7 |
| | 250 | 4.0 | 6.5 | 5.2 |

Minerals (+)
measured Mass Range: m/z = 313.8-315.3

TABLE 137

| | | protein concentration | | |
|---|---|---|---|---|
| FLJ25845 - | | (uM) | | |
| Cephaeline | | 0 | 11.9 | 23.8 |
| compound | 0 | 0.0 | 0.0 | 0.0 |
| concentration | 1 | 0.1 | 0.2 | 0.1 |
| (uM) | 10 | 0.3 | 0.6 | 0.6 |
| | 100 | 2.9 | 6.4 | 3.8 |
| | 250 | 9.7 | 9.8 | 14.4 |

Minerals (+)
measured Mass Range: m/z = 466.7-468.2

TABLE 138

| | | protein concentration | | |
|---|---|---|---|---|
| FLJ25845 - | | (uM) | | |
| Clenbuterol | | 0 | 11.9 | 23.8 |
| compound | 0 | 0.0 | 0.0 | 0.0 |
| concentration | 1 | 0.0 | 0.1 | 0.0 |
| (uM) | 10 | 0.2 | 0.3 | 0.4 |
| | 100 | 3.0 | 4.3 | 4.8 |
| | 250 | 8.9 | 6.3 | 12.7 |

Minerals (+)
measured Mass Range: m/z = 277.2-278.7

TABLE 139

| | | protein concentration | | |
|---|---|---|---|---|
| FLJ25845 - | | (uM) | | |
| Domperidone | | 0 | 11.9 | 23.8 |
| compound | 0 | 0.0 | 0.0 | 0.0 |
| concentration | 1 | 0.0 | 0.0 | 0.1 |
| (uM) | 10 | −0.1 | 0.1 | 0.2 |
| | 100 | 0.6 | 1.5 | 1.3 |
| | 250 | 1.5 | 5.5 | 5.7 |

Minerals (+)
measured Mass Range: m/z = 425.9-427.4

TABLE 140

| | | protein concentration | | |
|---|---|---|---|---|
| FLJ25845 - | | (uM) | | |
| Minocycline | | 0 | 11.9 | 23.8 |
| compound | 0 | 0.0 | 0.0 | 0.0 |
| concentration | 1 | 0.1 | 0.1 | 0.2 |
| (uM) | 10 | 0.3 | −0.1 | 1.0 |
| | 100 | 4.3 | 10.8 | 8.5 |
| | 250 | 18.9 | 19.7 | 20.3 |

Minerals (+)
measured Mass Range: m/z = 457.5-459

TABLE 141

| | | protein concentration | | |
|---|---|---|---|---|
| FLJ25845 - | | (uM) | | |
| Moxalactam | | 0 | 11.9 | 23.8 |
| compound | 0 | 0.0 | 0.0 | 0.0 |
| concentration | 1 | −0.4 | 1.2 | 0.0 |
| (uM) | 10 | 1.0 | 1.1 | 1.7 |
| | 100 | 15.8 | 17.0 | 19.5 |
| | 250 | 27.2 | 46.1 | 42.7 |

Minerals (+)
measured Mass Range: m/z = 520.5-522

TABLE 142A

| | | protein concentration | | |
|---|---|---|---|---|
| FLJ23662 - | | (uM) | | |
| Glibenclamide | | 0 | 23.8 | 47.5 |
| compound | 0 | 0.0 | 0.0 | 0.0 |
| concentration | 1 | 0.0 | 0.0 | 0.0 |
| (uM) | 10 | −0.1 | 0.0 | 0.0 |
| | 100 | 0.0 | 0.3 | 0.4 |
| | 250 | 0.7 | 0.7 | 4.3 |

Minerals (−)
measured Mass Range: m/z = 494-495.5

TABLE 142B

| | | protein concentration | | |
|---|---|---|---|---|
| FLJ23662 - | | (uM) | | |
| Glibenclamide | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.0 | 0.0 |
| | 100 | 2.1 | 0.9 | 1.8 |
| | 250 | 0.0 | 9.7 | 12.3 |

Minerals (+)
measured Mass Range: m/z = 494-495.5

TABLE 143A

| | | protein concentration | | |
|---|---|---|---|---|
| FLJ23662 - Raloxifene | | (uM) | | |
| (Raloxifene hydrochloride) | | 0 | 23.8 | 47.5 |
| compound | 0 | 0.0 | 0.0 | 0.0 |
| concentration | 1 | 0.0 | 0.1 | 0.1 |
| (uM) | 10 | 0.0 | 0.0 | 0.0 |
| | 100 | 0.0 | 0.2 | 0.1 |
| | 250 | 0.0 | 0.8 | 1.5 |

Minerals (−)
measured Mass Range: m/z = 473.6-475.1

TABLE 143B

| | protein concentration | | |
|---|---|---|---|
| FLJ23662 - Raloxifene | (uM) | | |
| (Raloxifene hydrochloride) | 0 | 23.8 | 47.5 |

| | | | | |
|---|---|---|---|---|
| compound | 0 | 0.0 | 0.0 | 0.0 |
| concentration | 1 | −0.5 | −0.1 | 0.0 |
| (uM) | 10 | −0.5 | −0.1 | 0.0 |
| | 100 | −0.5 | −0.1 | 0.0 |
| | 250 | −0.5 | 0.9 | 2.5 |

Minerals (+)
measured Mass Range: m/z = 473.6-475.1

TABLE 144A

| | | protein concentration | | |
|---|---|---|---|---|
| FLJ23662 - | | (uM) | | |
| Clofazimine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 1.3 | 0.6 |
| | 250 | 0.0 | 3.2 | 0.9 |

Minerals (−)
measured Mass Range: m/z = 473.4-474.9

TABLE 144B

| | | protein concentration | | |
|---|---|---|---|---|
| FLJ23662 - | | (uM) | | |
| Clofazimine | | 0 | 23.8 | 47.5 |
| compound | 0 | 0.0 | 0.0 | 0.0 |
| concentration | 1 | −0.4 | −0.1 | 0.0 |
| (uM) | 10 | −0.4 | 0.0 | 0.0 |
| | 100 | −0.4 | 0.2 | 0.2 |
| | 250 | −0.4 | 0.7 | 0.3 |

Minerals (+)
measured Mass Range: m/z = 473.4-474.9

TABLE 145A

| | | protein concentration | | |
|---|---|---|---|---|
| FLJ12668 - | | (uM) | | |
| Albendazole | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.0 |
| | 100 | 0.0 | 0.0 | 0.1 |
| | 250 | 0.0 | 0.3 | 0.0 |

Minerals (−)
measured Mass Range: m/z = 265.3-266.8

TABLE 145B

| | | protein concentration | | |
|---|---|---|---|---|
| FLJ12668 - | | (uM) | | |
| Albendazole | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.1 | 0.1 |
| | 100 | −0.1 | 0.8 | 2.0 |
| | 250 | −0.1 | 0.4 | 1.1 |

Minerals (+)
measured Mass Range: m/z = 265.3-266.8

TABLE 146A

| | | protein concentration | | |
|---|---|---|---|---|
| FLJ90085 - | | (uM) | | |
| Bezafibrate | | 0 | 23.8 | 47.5 |
| compound | 0 | 0.0 | 0.0 | 0.0 |
| concentration | 1 | −0.1 | 0.0 | 0.2 |
| (uM) | 10 | 0.6 | 0.4 | 1.0 |
| | 100 | 5.3 | 5.8 | 5.3 |
| | 250 | 19.0 | 22.7 | 29.0 |

Minerals (−)
measured Mass Range: m/z = 361.8-363.3

TABLE 146B

| | | protein concentration | | |
|---|---|---|---|---|
| FLJ90085 - | | (uM) | | |
| Bezafibrate | | 0 | 23.8 | 47.5 |
| compound | 0 | 0.0 | 0.0 | 0.0 |
| concentration | 1 | 0.4 | 0.1 | −0.1 |
| (uM) | 10 | 0.9 | 0.9 | 1.2 |
| | 100 | 7.6 | 11.1 | 10.2 |
| | 250 | 31.9 | 30.4 | 37.9 |

Minerals (+)
measured Mass Range: m/z = 361.8-363.3

TABLE 147A

| | | protein concentration | | |
|---|---|---|---|---|
| FLJ90364 - | | (uM) | | |
| Pirenzepine | | 0 | 23.8 | 47.5 |
| compound | 0 | 0.0 | 0.0 | 0.0 |
| concentration | 1 | 0.0 | 0.1 | 0.1 |
| (uM) | 10 | 0.5 | 0.4 | 0.6 |
| | 100 | 6.3 | 11.2 | 9.4 |
| | 250 | 16.9 | 28.2 | 28.5 |

Minerals (−)
measured Mass Range: m/z = 351.4-352.9

TABLE 147B

| | | protein concentration | | |
|---|---|---|---|---|
| FLJ90364 - | | (uM) | | |
| Pirenzepine | | 0 | 23.8 | 47.5 |
| compound | 0 | 0.0 | 0.0 | 0.0 |
| concentration | 1 | 0.2 | 0.1 | −0.7 |
| (uM) | 10 | 0.5 | 0.9 | 1.3 |
| | 100 | 10.6 | 9.4 | 14.3 |
| | 250 | 23.5 | 10.0 | 34.6 |

Minerals (+)
measured Mass Range: m/z = 351.4-352.9

TABLE 148

| | | protein concentration | | |
|---|---|---|---|---|
| FLJ90401 - | | (uM) | | |
| Rescinnamine | | 0 | 23.8 | 47.5 |
| compound | 0 | 0.0 | 0.0 | 0.0 |
| concentration | 1 | 0.2 | 0.0 | −1.2 |
| (uM) | 10 | −0.3 | −0.4 | −0.4 |
| | 100 | −1.2 | −1.0 | −1.2 |
| | 250 | −0.9 | −0.3 | 1.7 |

Minerals (+)
measured Mass Range: m/z = 634.7-636.2

TABLE 149A

| | | protein concentration | | |
|---|---|---|---|---|
| FLJ25526 - | | (uM) | | |
| Benzbromarone | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 0.0 | 3.4 |
| | 250 | 0.0 | 0.1 | 2.9 |

Minerals (−)
measured Mass Range: m/z = 424.1-425.6

TABLE 149B

| | | protein concentration | | |
|---|---|---|---|---|
| FLJ25526 - | | (uM) | | |
| Benzbromarone | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.8 |
| | 100 | 0.0 | 12.8 | 40.8 |
| | 250 | 0.0 | 9.6 | 78.4 |

Minerals (+)
measured Mass Range: m/z = 424.1-425.6

TABLE 150

| | | protein concentration | | |
|---|---|---|---|---|
| FLJ25526 - | | (uM) | | |
| Pranlukast | | 0 | 23.8 | 47.5 |
| compound | 0 | 0.0 | 0.0 | 0.0 |
| concentration | 1 | 0.0 | 0.0 | 0.1 |
| (uM) | 10 | 0.0 | 0.2 | 0.5 |
| | 100 | 0.0 | 6.0 | 28.7 |
| | 250 | 0.8 | 6.9 | 35.5 |

Minerals (+)
measured Mass Range: m/z = 481.5-483

TABLE 151A

| | | protein concentration | | |
|---|---|---|---|---|
| FLJ25526 - | | (uM) | | |
| Mifepristone | | 0 | 23.8 | 47.5 |
| compound | 0 | 0.0 | 0.0 | 0.0 |
| concentration | 1 | 0.1 | 0.0 | −0.2 |
| (uM) | 10 | 0.0 | 0.2 | 0.1 |
| | 100 | 0.2 | 0.5 | 0.7 |
| | 250 | 0.0 | 0.7 | 0.1 |

Minerals (−)
measured Mass Range: m/z = 429.6-431.1

TABLE 151B

| | | protein concentration | | |
|---|---|---|---|---|
| FLJ25526 - | | (uM) | | |
| Mifepristone | | 0 | 23.8 | 47.5 |
| compound | 0 | 0.0 | 0.0 | 0.0 |
| concentration | 1 | 0.0 | 0.1 | −0.3 |
| (uM) | 10 | 0.0 | 0.2 | 0.3 |
| | 100 | 0.6 | 0.8 | 1.4 |
| | 250 | 0.5 | 3.6 | 3.1 |

Minerals (+)
measured Mass Range: m/z = 429.6-431.1

TABLE 152A

| | | protein concentration (uM) | | |
|---|---|---|---|---|
| FLJ25526 - Mestanolone | | 0 | 23.8 | 47.5 |
| compound | 0 | 0.0 | 0.0 | 0.0 |
| concentration | 1 | −0.8 | 0.0 | −0.4 |
| (uM) | 10 | 4.2 | 0.1 | −0.1 |
| | 100 | 1.1 | 1.2 | 1.3 |
| | 250 | 0.6 | 2.2 | 3.4 |

Minerals (−)
measured Mass Range: m/z = 304.5-306

TABLE 152B

| | | protein concentration (uM) | | |
|---|---|---|---|---|
| FLJ25526 - Mestanolone | | 0 | 23.8 | 47.5 |
| compound | 0 | 0.0 | 0.0 | 0.0 |
| concentration | 1 | −2.3 | −3.5 | −8.6 |
| (uM) | 10 | −3.4 | −2.4 | −8.3 |
| | 100 | −3.3 | 0.1 | −6.7 |
| | 250 | −1.2 | −0.4 | −4.2 |

Minerals (+)
measured Mass Range: m/z = 304.5-306

TABLE 153A

| | | protein concentration (uM) | | |
|---|---|---|---|---|
| FLJ46896 - Hydroxytacrine (R,S) | | 0 | 23.8 | 47.5 |
| compound | 0 | 0.0 | 0.0 | 0.0 |
| concentration | 1 | 0.0 | 0.1 | 0.1 |
| (uM) | 10 | 0.3 | 1.2 | 1.4 |
| | 100 | 3.9 | 7.2 | 14.2 |
| | 250 | 7.9 | 6.3 | 21.9 |

Minerals (−)
measured Mass Range: m/z = 214.3-215.8

TABLE 153B

| | | protein concentration (uM) | | |
|---|---|---|---|---|
| FLJ46896 - Hydroxytacrine (R,S) | | 0 | 23.8 | 47.5 |
| compound | 0 | 0.0 | 0.0 | 0.0 |
| concentration | 1 | −0.4 | −0.1 | 0.0 |
| (uM) | 10 | −0.2 | 0.1 | 1.7 |
| | 100 | 4.5 | 4.4 | 7.9 |
| | 250 | 5.7 | 12.4 | 17.4 |

Minerals (+)
measured Mass Range: m/z = 214.3-215.8

TABLE 154A

| FLJ46896 - Metergotamine | | protein concentration (uM) | | |
|---|---|---|---|---|
| (Metergoline) | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 0.5 | 0.1 |
| | 250 | 0.2 | 0.6 | 1.8 |

Minerals (−)
measured Mass Range: m/z = 403.5-405

TABLE 154B

| FLJ46896 - Metergotamine | | protein concentration (uM) | | |
|---|---|---|---|---|
| (Metergoline) | | 0 | 23.8 | 47.5 |
| compound | 0 | 0.0 | 0.0 | 0.0 |
| concentration | 1 | −0.6 | −0.1 | 0.0 |
| (uM) | 10 | −0.6 | −0.1 | 0.0 |
| | 100 | −0.6 | 0.4 | 0.3 |
| | 250 | 0.6 | 1.5 | 4.0 |

Minerals (+)
measured Mass Range: m/z = 403.5-405

TABLE 155A

| | | protein concentration (uM) | | |
|---|---|---|---|---|
| FLJ46896 - Metaproterenol | | 0 | 23.8 | 47.5 |
| compound | 0 | 0.0 | 0.0 | 0.0 |
| concentration | 1 | 0.0 | 0.0 | 0.1 |
| (uM) | 10 | 0.0 | 0.2 | 0.5 |
| | 100 | 6.5 | 7.4 | 6.4 |
| | 250 | 12.2 | 9.8 | 19.4 |

Minerals (−)
measured Mass Range: m/z = 211.1-212.6

TABLE 155B

| | | protein concentration (uM) | | |
|---|---|---|---|---|
| FLJ46896 - Metaproterenol | | 0 | 23.8 | 47.5 |
| compound | 0 | 0.0 | 0.0 | 0.0 |
| concentration | 1 | −0.1 | 0.0 | 0.0 |
| (uM) | 10 | 0.0 | 0.1 | 0.5 |
| | 100 | 5.3 | 7.9 | 10.8 |
| | 250 | 9.5 | 22.7 | 21.0 |

Minerals (+)
measured Mass Range: m/z = 211.1-212.6

TABLE 156A

| FLJ46856 - Eburnamonine | | protein concentration (uM) | | |
|---|---|---|---|---|
| (Eburnamonine (−)) | | 0 | 23.8 | 47.5 |
| compound | 0 | 0.0 | 0.0 | 0.0 |
| concentration | 1 | −0.2 | −0.1 | −0.1 |
| (uM) | 10 | −0.3 | 0.0 | 0.1 |
| | 100 | 0.8 | 2.2 | 2.8 |
| | 250 | 3.1 | 3.3 | 3.2 |

Minerals (−)
measured Mass Range: m/z = 294.4-295.9

TABLE 156B

| FLJ46856 - Eburnamonine | | protein concentration (uM) | | |
|---|---|---|---|---|
| (Eburnamonine (−)) | | 0 | 23.8 | 47.5 |
| compound | 0 | 0.0 | 0.0 | 0.0 |
| concentration | 1 | 0.0 | 0.0 | 0.0 |
| (uM) | 10 | 0.1 | 0.2 | 0.3 |
| | 100 | 1.0 | 4.5 | 3.9 |
| | 250 | 4.9 | 3.3 | 7.5 |

Minerals (+)
measured Mass Range: m/z = 294.4-295.9

TABLE 157A

| FLJ46856 - Levobunolol | | protein concentration (uM) | | |
|---|---|---|---|---|
| (Levobunolol hydrochloride (+)) | | 0 | 23.8 | 47.5 |
| compound | 0 | 0.0 | 0.0 | 0.0 |
| concentration | 1 | 0.0 | 0.0 | 0.0 |
| (uM) | 10 | 0.2 | 0.4 | 0.6 |
| | 100 | 4.5 | 2.9 | 6.4 |
| | 250 | 10.0 | 18.1 | 18.2 |

Minerals (−)
measured Mass Range: m/z = 291.4-292.9

TABLE 157B

| FLJ46856 - Levobunolol | | protein concentration (uM) | | |
|---|---|---|---|---|
| (Levobunolol hydrochloride (+)) | | 0 | 23.8 | 47.5 |
| compound | 0 | 0.0 | 0.0 | 0.0 |
| concentration | 1 | 0.0 | 0.0 | 0.0 |
| (uM) | 10 | 0.2 | 0.4 | 0.4 |
| | 100 | 4.7 | 15.9 | 6.0 |
| | 250 | 20.2 | 15.3 | 11.8 |

Minerals (+)
measured Mass Range: m/z = 291.4-292.9

TABLE 158A

| | | protein concentration (uM) | | |
|---|---|---|---|---|
| FLJ90345 - Norharman | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.1 | 0.1 | 0.1 |
| | 250 | 0.4 | 0.2 | 1.9 |

Minerals (−)
measured Mass Range: m/z = 168.2-169.7

TABLE 158B

| | | protein concentration (uM) | | |
|---|---|---|---|---|
| FLJ90345 - Norharman | | 0 | 23.8 | 47.5 |
| compound | 0 | 0.0 | 0.0 | 0.0 |
| concentration | 1 | −0.1 | 0.0 | 0.0 |
| (uM) | 10 | 0.0 | 0.0 | 0.0 |
| | 100 | 0.1 | 0.1 | 0.1 |
| | 250 | 1.1 | 0.3 | 1.0 |

Minerals (+)
measured Mass Range: m/z = 168.2-169.7

TABLE 159A

| | | protein concentration (uM) | | |
|---|---|---|---|---|
| FLJ90345 - Pyrilamine | | 0 | 23.8 | 47.5 |
| compound | 0 | 0.0 | 0.0 | 0.0 |
| concentration | 1 | 0.0 | 0.0 | 0.0 |
| (uM) | 10 | 0.3 | 0.2 | 0.2 |
| | 100 | 4.2 | 3.3 | 1.2 |
| | 250 | 10.8 | 5.6 | 18.9 |

Minerals (−)
measured Mass Range: m/z = 285.5-287

TABLE 159B

| | | protein concentration (uM) | | |
|---|---|---|---|---|
| FLJ90345 - Pyrilamine | | 0 | 23.8 | 47.5 |
| compound | 0 | 0.0 | 0.0 | 0.0 |
| concentration | 1 | −0.1 | 0.1 | 0.1 |
| (uM) | 10 | 0.1 | 0.1 | 0.1 |
| | 100 | 9.5 | 5.1 | 0.8 |
| | 250 | 13.2 | 8.0 | 13.5 |

Minerals (+)
measured Mass Range: m/z = 285.5-287

TABLE 160A

| | | protein concentration (uM) | | |
|---|---|---|---|---|
| FLJ26550 - Celestin blue | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 0.2 | 0.3 | 0.1 |
| | 250 | 0.0 | 1.4 | 0.1 |

Minerals (−)
measured Mass Range: m/z = 328.4-329.9

TABLE 160B

| | | protein concentration (uM) | | |
|---|---|---|---|---|
| FLJ26550 - Celestin blue | | 0 | 23.8 | 47.5 |
| compound | 0 | 0.0 | 0.0 | 0.0 |
| concentration | 1 | 0.1 | 0.0 | 0.1 |
| (uM) | 10 | 0.0 | 0.1 | 0.3 |
| | 100 | 1.2 | 1.7 | 4.3 |
| | 250 | 2.0 | 6.9 | 12.1 |

Minerals (+)
measured Mass Range: m/z = 328.4-329.9

TABLE 161A

| FLJ26550 - Nitrarine | | protein concentration (uM) | | |
|---|---|---|---|---|
| (Nitrarine dihydrochloride) | | 0 | 23.8 | 47.5 |
| compound | 0 | 0.0 | 0.0 | 0.0 |
| concentration | 1 | 0.0 | 0.0 | 0.0 |
| (uM) | 10 | 0.4 | 0.6 | 0.0 |
| | 100 | 5.1 | 7.4 | 6.0 |
| | 250 | 21.7 | 12.6 | 17.2 |

Minerals (−)
measured Mass Range: m/z = 307.4-308.9

TABLE 161B

| FLJ26550 - Nitrarine | | protein concentration (uM) | | |
|---|---|---|---|---|
| (Nitrarine dihydrochloride) | | 0 | 23.8 | 47.5 |
| compound | 0 | 0.0 | 0.0 | 0.0 |
| concentration | 1 | −0.1 | 0.0 | 0.0 |
| (uM) | 10 | 0.1 | 0.3 | 0.8 |
| | 100 | 10.9 | 5.2 | 3.2 |
| | 250 | 10.4 | 16.2 | 25.2 |

Minerals (+)
measured Mass Range: m/z = 307.4-308.9

TABLE 162A

| | | protein concentration (uM) | | |
|---|---|---|---|---|
| FLJ90015 - Clemizole | | 0 | 11.9 | 23.8 |
| compound | 0 | 0.0 | 0.0 | 0.0 |
| concentration | 1 | 0.0 | 0.0 | 0.0 |
| (uM) | 10 | 0.1 | 0.0 | 0.0 |
| | 100 | 0.7 | 1.2 | 1.0 |
| | 250 | 4.4 | 9.1 | 9.5 |

Minerals (−)
measured Mass Range: m/z = 325.8-327.3

TABLE 162B

| | | protein concentration (uM) | | |
|---|---|---|---|---|
| FLJ90015 - Clemizole | | 0 | 11.9 | 23.8 |
| compound | 0 | 0.0 | 0.0 | 0.0 |
| concentration | 1 | 0.0 | 0.0 | 0.0 |
| (uM) | 10 | 0.1 | 0.2 | 0.1 |
| | 100 | 2.6 | 5.2 | 4.5 |
| | 250 | 18.1 | 7.2 | 7.1 |

Minerals (+)
measured Mass Range: m/z = 325.8-327.3

TABLE 163A

| | | protein concentration (uM) | | |
|---|---|---|---|---|
| FLJ39454 - Clobazam | | 0 | 23.8 | 47.5 |
| compound | 0 | 0.0 | 0.0 | 0.0 |
| concentration | 1 | 1.4 | −1.1 | 2.0 |
| (uM) | 10 | 0.0 | 0.5 | 1.0 |
| | 100 | 5.6 | 4.1 | 5.5 |
| | 250 | 14.3 | 12.5 | 12.6 |

Minerals (−)
measured Mass Range: m/z = 300.7-302.2

TABLE 163B

| | | protein concentration (uM) | | |
|---|---|---|---|---|
| FLJ39454 - Clobazam | | 0 | 23.8 | 47.5 |
| compound | 0 | 0.0 | 0.0 | 0.0 |
| concentration | 1 | −2.5 | −6.0 | −1.4 |
| (uM) | 10 | −1.7 | −2.0 | −1.1 |
| | 100 | 1.5 | 2.0 | 4.1 |
| | 250 | −9.6 | 10.2 | 10.0 |

Minerals (+)
measured Mass Range: m/z = 300.7-302.2

TABLE 164A

| | | protein concentration (uM) | | |
|---|---|---|---|---|
| FLJ45115 - Josamycin | | 0 | 11.9 | 23.8 |
| compound | 0 | 0.0 | 0.0 | 0.0 |
| concentration | 1 | 0.1 | −0.8 | −0.1 |
| (uM) | 10 | 1.7 | 3.1 | 2.2 |
| | 100 | 23.9 | 29.6 | 28.2 |
| | 250 | 59.5 | 73.5 | 80.0 |

Minerals (−)
measured Mass Range: m/z = 828-829.5

TABLE 164B

| | | protein concentration (uM) | | |
|---|---|---|---|---|
| FLJ45115 - Josamycin | | 0 | 11.9 | 23.8 |
| compound | 0 | 0.0 | 0.0 | 0.0 |
| concentration | 1 | 0.1 | −0.6 | −0.6 |
| (uM) | 10 | 2.1 | 3.6 | 3.0 |
| | 100 | 26.0 | 31.7 | 31.0 |
| | 250 | 36.2 | 70.7 | 87.0 |

Minerals (+)
measured Mass Range: m/z = 828-829.5

TABLE 165A

| | | protein concentration (uM) | | |
|---|---|---|---|---|
| FLJ45115 - Oxytocin | | 0 | 11.9 | 23.8 |
| compound | 0 | 0.0 | 0.0 | 0.0 |
| concentration | 1 | 0.0 | −0.2 | 1.1 |
| (uM) | 10 | 0.6 | 0.5 | 0.6 |
| | 100 | 4.8 | 9.3 | 9.6 |
| | 250 | 16.7 | 15.6 | 20.4 |

Minerals (−)
measured Mass Range: m/z = 1007.2-1008.7

TABLE 165B

| | | protein concentration (uM) | | |
|---|---|---|---|---|
| FLJ45115 - Oxytocin | | 0 | 11.9 | 23.8 |
| compound | 0 | 0.0 | 0.0 | 0.0 |
| concentration | 1 | 0.1 | −2.5 | −0.4 |
| (uM) | 10 | 0.6 | 0.3 | 0.2 |
| | 100 | 6.9 | 4.8 | 7.9 |
| | 250 | 29.0 | 35.1 | 15.1 |

Minerals (+)
measured Mass Range: m/z = 1007.2-1008.7

TABLE 166A

| | | protein concentration (uM) | | |
|---|---|---|---|---|
| FLJ45115 - Clarithromycin | | 0 | 11.9 | 23.8 |
| compound | 0 | 0.0 | 0.0 | 0.0 |
| concentration | 1 | 0.1 | 0.0 | 0.0 |
| (uM) | 10 | 0.9 | 1.1 | 0.8 |
| | 100 | 10.7 | 11.7 | 7.7 |
| | 250 | 48.4 | 29.0 | 28.0 |

Minerals (−)
measured Mass Range: m/z = 748-749.5

TABLE 166B

| | | protein concentration (uM) | | |
|---|---|---|---|---|
| FLJ45115 - Clarithromycin | | 0 | 11.9 | 23.8 |
| compound | 0 | 0.0 | 0.0 | 0.0 |
| concentration | 1 | 0.1 | −0.2 | 0.0 |
| (uM) | 10 | 0.8 | 1.1 | 1.0 |
| | 100 | 13.2 | 11.1 | 8.5 |
| | 250 | 18.6 | 41.7 | 33.0 |

Minerals (+)
measured Mass Range: m/z = 748-749.5

TABLE 167A

| | | protein | | |
|---|---|---|---|---|
| FLJ90066 - | | concentration (uM) | | |
| Leuprolide | | 0 | 11.9 | 23.8 |
| compound | 0 | 0.0 | 0.0 | 0.0 |
| concentration | 1 | −0.1 | 0.0 | 0.0 |
| (uM) | 10 | 0.2 | 0.5 | 0.5 |
| | 100 | 6.6 | 12.0 | 8.8 |
| | 250 | 22.2 | 28.1 | 33.5 |

Minerals (−)
measured Mass Range: m/z = 1209.4-1210.9

TABLE 167B

| | | protein | | |
|---|---|---|---|---|
| FLJ90066 - | | concentration (uM) | | |
| Leuprolide | | 0 | 11.9 | 23.8 |
| compound | 0 | 0.0 | 0.0 | 0.0 |
| concentration | 1 | 0.0 | 0.0 | −0.6 |
| (uM) | 10 | 0.5 | 0.4 | 0.1 |
| | 100 | 11.6 | 11.1 | 11.8 |
| | 250 | 26.7 | 48.7 | 31.7 |

Minerals (+)
measured Mass Range: m/z = 1209.4-1210.9

TABLE 168A

| | | protein | | |
|---|---|---|---|---|
| FLJ90066 - | | concentration (uM) | | |
| Cyclosporin A | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 0.0 | 0.1 |
| | 250 | 0.0 | 13.5 | 5.2 |

Minerals (−)
measured Mass Range: m/z = 1202.6-1204.1

TABLE 168B

| | | protein | | |
|---|---|---|---|---|
| FLJ90066 - | | concentration (uM) | | |
| Cyclosporin A | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 0.2 | 0.0 |
| | 250 | 0.0 | 1.2 | 1.1 |

Minerals (+)
measured Mass Range: m/z = 1202.6-1204.1

TABLE 169A

| | | protein | | |
|---|---|---|---|---|
| FLJ37995 - | | concentration (uM) | | |
| Diclofenamide | | 0 | 23.8 | 47.5 |
| compound | 0 | 0.0 | 0.0 | 0.0 |
| concentration | 1 | −0.3 | 1.3 | 1.6 |
| (uM) | 10 | −0.1 | 3.6 | 6.6 |
| | 100 | 0.9 | 9.5 | 12.7 |
| | 250 | 2.6 | 11.7 | 18.7 |

Minerals (−)
measured Mass Range: m/z = 305.2-306.7

TABLE 169B

| | | protein | | |
|---|---|---|---|---|
| FLJ37995 - | | concentration (uM) | | |
| Diclofenamide | | 0 | 23.8 | 47.5 |
| compound | 0 | 0.0 | 0.0 | 0.0 |
| concentration | 1 | 0.3 | 1.5 | 1.0 |
| (uM) | 10 | −0.3 | 16.1 | 20.4 |
| | 100 | 0.4 | 27.6 | 62.3 |
| | 250 | 2.5 | 27.3 | 69.9 |

Minerals (+)
measured Mass Range: m/z = 305.2-306.7

TABLE 170A

| | | protein | | |
|---|---|---|---|---|
| FLJ37995 - | | concentration (uM) | | |
| Benzthiazide | | 0 | 23.8 | 47.5 |
| compound | 0 | 0.0 | 0.0 | 0.0 |
| concentration | 1 | 0.0 | 0.6 | 0.7 |
| (uM) | 10 | 0.0 | 1.4 | 2.6 |
| | 100 | 0.1 | 2.6 | 4.0 |
| | 250 | 0.1 | 2.8 | 5.3 |

Minerals (−)
measured Mass Range: m/z = 431.9-433.4

TABLE 170B

| | | protein | | |
|---|---|---|---|---|
| FLJ37995 - | | concentration (uM) | | |
| Benzthiazide | | 0 | 23.8 | 47.5 |
| compound | 0 | 0.0 | 0.0 | 0.0 |
| concentration | 1 | 0.0 | 0.9 | 0.8 |
| (uM) | 10 | 0.0 | 5.9 | 7.6 |
| | 100 | 0.1 | 10.3 | 35.8 |
| | 250 | 0.1 | 10.3 | 35.2 |

Minerals (+)
measured Mass Range: m/z = 431.9-433.4

TABLE 171A

| | | protein | | |
|---|---|---|---|---|
| FLJ26058 - | | concentration (uM) | | |
| Hydroxychloroquine | | 0 | 23.8 | 47.5 |
| compound | 0 | 0.0 | 0.0 | 0.0 |
| concentration | 1 | 0.0 | 0.1 | 0.0 |
| (uM) | 10 | 0.3 | 0.5 | 0.8 |
| | 100 | 3.3 | 14.7 | 13.8 |
| | 250 | 7.5 | 17.7 | 18.6 |

Minerals (−)
measured Mass Range: m/z = 335.9-337.4

TABLE 171B

| | | protein | | |
|---|---|---|---|---|
| FLJ26058 - | | concentration (uM) | | |
| Hydroxychloroquine | | 0 | 23.8 | 47.5 |
| compound | 0 | 0.0 | 0.0 | 0.0 |
| concentration | 1 | −0.8 | −0.3 | −0.2 |
| (uM) | 10 | −0.7 | −0.2 | 0.7 |
| | 100 | 5.5 | 3.7 | 15.7 |
| | 250 | 10.8 | 18.6 | 13.7 |

Minerals (+)
measured Mass Range: m/z = 335.9-337.4

TABLE 172

| | | protein | | |
|---|---|---|---|---|
| FLJ46369 - | | concentration (uM) | | |
| Benzbromarone | | 0 | 11.9 | 23.8 |
| compound | 0 | 0.0 | 0.0 | 0.0 |
| concentration | 1 | 0.0 | 0.1 | 0.0 |
| (uM) | 10 | 0.0 | 0.3 | 1.2 |
| | 100 | −0.2 | 3.6 | 6.8 |
| | 250 | −0.3 | 12.3 | 61.8 |

Minerals (+)
measured Mass Range: m/z = 424.1-425.6

TABLE 173

| | | protein | | |
|---|---|---|---|---|
| FLJ46369 - | | concentration (uM) | | |
| Benzethonium | | 0 | 11.9 | 23.8 |
| compound | 0 | 0.0 | 0.0 | 0.0 |
| concentration | 1 | 0.0 | 0.1 | 0.2 |
| (uM) | 10 | 0.0 | 0.1 | 1.2 |
| | 100 | 0.1 | 8.7 | 18.5 |
| | 250 | 2.6 | 21.1 | 44.4 |

Minerals (+)
measured Mass Range: m/z = 412.6-414.1

TABLE 174

| | | protein | | |
|---|---|---|---|---|
| FLJ46369 - | | concentration (uM) | | |
| Clofazimine | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.2 |
| | 100 | 0.0 | 2.4 | 5.9 |
| | 250 | 0.0 | 11.8 | 36.5 |

Minerals (+)
measured Mass Range: m/z = 473.4-474.9

TABLE 175

| | | protein | | |
|---|---|---|---|---|
| FLJ46369 - | | concentration (uM) | | |
| Domperidone | | 0 | 11.9 | 23.8 |
| compound | 0 | 0.0 | 0.0 | 0.0 |
| concentration | 1 | 0.1 | 0.1 | 0.0 |
| (uM) | 10 | 0.2 | 0.4 | 0.2 |
| | 100 | 1.2 | 3.7 | 4.0 |
| | 250 | 3.6 | 6.7 | 7.9 |

Minerals (+)
measured Mass Range: m/z = 425.9-427.4

TABLE 176

| | | protein | | |
|---|---|---|---|---|
| FLJ46369 - | | concentration (uM) | | |
| Doxazosin | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.2 | 0.2 |
| | 100 | 0.7 | 1.6 | 2.1 |
| | 250 | 0.7 | 3.1 | 3.7 |

Minerals (+)
measured Mass Range: m/z = 451.5-453

TABLE 177

| | | protein | | |
|---|---|---|---|---|
| FLJ46369 - | | concentration (uM) | | |
| Gramicidin | | 0 | 11.9 | 23.8 |
| compound | 0 | 0.0 | 0.0 | 0.0 |
| concentration | 1 | −0.3 | 0.1 | 0.1 |
| (uM) | 10 | −0.7 | 1.6 | 1.2 |
| | 100 | −0.5 | 6.9 | 11.2 |
| | 250 | −0.9 | 15.0 | 22.1 |

Minerals (+)
measured Mass Range: m/z = 1882.3-1883.8

TABLE 178

| | | protein | | |
|---|---|---|---|---|
| FLJ46369 - | | concentration (uM) | | |
| Ergocryptine-alpha | | 0 | 11.9 | 23.8 |
| compound | 0 | 0.0 | 0.0 | 0.0 |
| concentration | 1 | −0.1 | 0.1 | 0.0 |
| (uM) | 10 | −0.1 | 0.2 | 0.1 |
| | 100 | 0.5 | 4.2 | 4.0 |
| | 250 | 1.8 | 13.9 | 18.4 |

Minerals (+)
measured Mass Range: m/z = 575.7-577.2

TABLE 179

| | | protein | | |
|---|---|---|---|---|
| FLJ46369 - | | concentration (uM) | | |
| Bicalutamide | | 0 | 11.9 | 23.8 |
| compound | 0 | 0.0 | 0.0 | 0.0 |
| concentration | 1 | 0.0 | 0.0 | 0.0 |
| (uM) | 10 | 0.2 | 0.2 | 0.2 |
| | 100 | 1.5 | 5.2 | 5.1 |
| | 250 | 0.6 | 24.1 | 17.5 |

Minerals (+)
measured Mass Range: m/z = 430.4-431.9

TABLE 180

| | | protein | | |
|---|---|---|---|---|
| FLJ46369 - | | concentration (uM) | | |
| Rescinnamine | | 0 | 11.9 | 23.8 |
| compound | 0 | 0.0 | 0.0 | 0.0 |
| concentration | 1 | −0.1 | −0.1 | 0.3 |
| (uM) | 10 | −0.5 | 0.2 | 0.1 |
| | 100 | 0.0 | 0.3 | 0.4 |
| | 250 | −0.1 | 0.7 | 0.9 |

Minerals (+)
measured Mass Range: m/z = 634.7-636.2

TABLE 181

| | | protein | | |
|---|---|---|---|---|
| FLJ46369 - | | concentration (uM) | | |
| Saquinavir | | 0 | 11.9 | 23.8 |
| compound | 0 | 0.0 | 0.0 | 0.0 |
| concentration | 1 | 0.0 | 0.0 | 0.0 |
| (uM) | 10 | 0.3 | 0.4 | 0.4 |
| | 100 | 4.4 | 3.4 | 4.6 |
| | 250 | 10.4 | 9.9 | 13.8 |

Minerals (+)
measured Mass Range: m/z = 670.9-672.4

TABLE 182

| | | protein | | |
|---|---|---|---|---|
| FLJ46369 - | | concentration (uM) | | |
| Syrosingopine | | 0 | 11.9 | 23.8 |
| compound | 0 | 0.0 | 0.0 | 0.0 |
| concentration | 1 | 0.2 | −0.2 | −0.2 |
| (uM) | 10 | 0.1 | 0.1 | −0.2 |
| | 100 | −0.1 | 0.8 | 0.8 |
| | 250 | −0.1 | 33.1 | 24.7 |

Minerals (+)
measured Mass Range: m/z = 666.7-668.2

TABLE 183

| | | protein | | |
|---|---|---|---|---|
| FLJ46369 - | | concentration (uM) | | |
| Pranlukast | | 0 | 11.9 | 23.8 |
| compound | 0 | 0.0 | 0.0 | 0.0 |
| concentration | 1 | −0.1 | 0.1 | 0.1 |
| (uM) | 10 | −0.1 | 0.2 | 0.3 |
| | 100 | 0.0 | 11.6 | 16.3 |
| | 250 | 1.0 | 87.3 | 74.1 |

Minerals (+)
measured Mass Range: m/z = 481.5-483

TABLE 184A

| | | protein | | |
|---|---|---|---|---|
| FLJ16517 - | | concentration (uM) | | |
| Benzbromarone | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.2 |
| | 100 | 0.0 | 2.4 | 12.6 |
| | 250 | 0.0 | 17.9 | 83.5 |

Minerals (−)
measured Mass Range: m/z = 424.1-425.6

TABLE 184B

| | | protein | | |
|---|---|---|---|---|
| FLJ16517 - | | concentration (uM) | | |
| Benzbromarone | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.2 | 0.5 |
| | 100 | −0.2 | 6.8 | 40.2 |
| | 250 | −0.1 | 16.6 | 80.1 |

Minerals (+)
measured Mass Range: m/z = 424.1-425.6

TABLE 185A

| | | protein | | |
|---|---|---|---|---|
| FLJ16517 - | | concentration (uM) | | |
| Clofazimine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.2 |
| | 100 | 0.0 | 1.3 | 4.2 |
| | 250 | 0.0 | 4.3 | 5.2 |

Minerals (−)
measured Mass Range: m/z = 473.4-474.9

TABLE 185B

| | | protein | | |
|---|---|---|---|---|
| FLJ16517 - | | concentration (uM) | | |
| Clofazimine | | 0 | 23.8 | 47.5 |
| compound | 0 | 0.0 | 0.0 | 0.0 |
| concentration | 1 | −0.2 | 0.0 | 0.0 |
| (uM) | 10 | −0.2 | 0.1 | 0.2 |
| | 100 | −0.2 | 3.2 | 6.2 |
| | 250 | −0.2 | 8.2 | 12.0 |

Minerals (+)
measured Mass Range: m/z = 473.4-474.9

TABLE 186A

| | | protein | | |
|---|---|---|---|---|
| FLJ16517 - | | concentration (uM) | | |
| Domperidone | | 0 | 23.8 | 47.5 |
| compound | 0 | 0.0 | 0.0 | 0.0 |
| concentration | 1 | 0.0 | 0.0 | 0.0 |
| (uM) | 10 | 0.1 | 0.1 | 0.0 |
| | 100 | 0.8 | 1.3 | 1.0 |
| | 250 | 1.4 | 4.5 | 3.6 |

Minerals (−)
measured Mass Range: m/z = 425.9-427.4

TABLE 186B

| | | protein | | |
|---|---|---|---|---|
| FLJ16517 - | | concentration (uM) | | |
| Domperidone | | 0 | 23.8 | 47.5 |
| compound | 0 | 0.0 | 0.0 | 0.0 |
| concentration | 1 | 0.0 | 0.1 | −0.1 |
| (uM) | 10 | 0.1 | 0.1 | 0.1 |
| | 100 | 0.8 | 1.8 | 1.7 |
| | 250 | 2.8 | 2.8 | 4.0 |

Minerals (+)
measured Mass Range: m/z = 425.9-427.4

TABLE 187A

| | | protein | | |
|---|---|---|---|---|
| FLJ16517 - | | concentration (uM) | | |
| Nicardipine | | 0 | 23.8 | 47.5 |
| compound | 0 | 0.0 | 0.0 | 0.0 |
| concentration | 1 | 0.0 | 0.0 | 0.0 |
| (uM) | 10 | 0.1 | 0.3 | 0.4 |
| | 100 | 2.5 | 2.6 | 2.4 |
| | 250 | 4.1 | 6.8 | 9.0 |

Minerals (−)
measured Mass Range: m/z = 479.5-481

TABLE 187B

| | | protein | | |
|---|---|---|---|---|
| FLJ16517 - | | concentration (uM) | | |
| Nicardipine | | 0 | 23.8 | 47.5 |
| compound | 0 | 0.0 | 0.0 | 0.0 |
| concentration | 1 | −0.1 | 0.0 | 0.0 |
| (uM) | 10 | 0.1 | 0.1 | 0.2 |
| | 100 | 1.8 | 2.1 | 3.6 |
| | 250 | 6.6 | 3.2 | 7.6 |

Minerals (+)
measured Mass Range: m/z = 479.5-481

TABLE 188A

| | | protein concentration | | |
|---|---|---|---|---|
| | | (uM) | | |
| FLJ16517 - Quercetine | | 0 | 23.8 | 47.5 |
| compound | 0 | 0.0 | 0.0 | 0.0 |
| concentration | 1 | 0.1 | 0.0 | 0.0 |
| (uM) | 10 | 0.1 | 0.0 | 0.0 |
| | 100 | 0.1 | 0.3 | 0.7 |
| | 250 | 0.1 | 0.6 | 1.8 |

Minerals (−)
measured Mass Range: m/z = 320.3-321.8

TABLE 188B

| | | protein concentration | | |
|---|---|---|---|---|
| | | (uM) | | |
| FLJ16517 - Quercetine | | 0 | 23.8 | 47.5 |
| compound | 0 | 0.0 | 0.0 | 0.0 |
| concentration | 1 | −0.1 | 0.1 | 0.0 |
| (uM) | 10 | −0.1 | 0.6 | 0.7 |
| | 100 | −0.1 | 19.9 | 21.6 |
| | 250 | 0.1 | 50.3 | 41.2 |

Minerals (+)
measured Mass Range: m/z = 320.3-321.8

TABLE 189A

| | | protein concentration | | |
|---|---|---|---|---|
| | | (uM) | | |
| FLJ16517 - Ebastine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.0 | 1.3 | 1.0 |
| | 250 | 0.1 | 9.2 | 11.8 |

Minerals (−)
measured Mass Range: m/z = 469.7-471.2

TABLE 189B

| | | protein concentration | | |
|---|---|---|---|---|
| | | (uM) | | |
| FLJ16517 - Ebastine | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.0 | 0.1 |
| | 100 | −0.1 | 1.0 | 0.7 |
| | 250 | 0.0 | 18.4 | 12.8 |

Minerals (+)
measured Mass Range: m/z = 469.7-471.2

TABLE 190A

| | | protein concentration | | |
|---|---|---|---|---|
| FLJ16517 - | | (uM) | | |
| Actinomycin D | | 0 | 23.8 | 47.5 |
| compound | 0 | 0.0 | 0.0 | 0.0 |
| concentration | 1 | 0.0 | 0.2 | 0.3 |
| (uM) | 10 | 0.3 | 1.0 | 2.2 |
| | 100 | 7.5 | 9.9 | 11.6 |
| | 250 | 26.0 | 26.2 | 31.4 |

Minerals (−)
measured Mass Range: m/z = 1255.4-1256.9

TABLE 190B

| | | protein concentration | | |
|---|---|---|---|---|
| FLJ16517 - | | (uM) | | |
| Actinomycin D | | 0 | 23.8 | 47.5 |
| compound | 0 | 0.0 | 0.0. | 0.0 |
| concentration | 1 | −0.3 | 0.0 | 1.0 |
| (uM) | 10 | 0.3 | 0.7 | 2.6 |
| | 100 | 6.6 | 11.9 | 13.2 |
| | 250 | 31.6 | 17.3 | 31.8 |

Minerals (+)
measured Mass Range: m/z = 1255.4-1256.9

TABLE 191A

| | | protein concentration | | |
|---|---|---|---|---|
| FLJ16517 - | | (uM) | | |
| Loperamide | | 0 | 23.8 | 47.5 |
| compound | 0 | 0.0 | 0.0 | 0.0 |
| concentration | 1 | 0.0 | 0.1 | 0.1 |
| (uM) | 10 | 0.2 | 0.6 | 1.6 |
| | 100 | 6.0 | 11.9 | 10.3 |
| | 250 | 28.1 | 29.6 | 21.7 |

Minerals (−)
measured Mass Range: m/z = 477-478.5

TABLE 191B

| | | protein concentration | | |
|---|---|---|---|---|
| FLJ16517 - | | (uM) | | |
| Loperamide | | 0 | 23.8 | 47.5 |
| compound | 0 | 0.0 | 0.0 | 0.0 |
| concentration | 1 | −0.2 | 0.0 | 0.0 |
| (uM) | 10 | 0.1 | 0.5 | 0.7 |
| | 100 | 5.7 | 6.6 | 7.5 |
| | 250 | 16.8 | 12.7 | 21.0 |

Minerals (+)
measured Mass Range: m/z = 477-478.5

TABLE 192

| | | protein concentration | | |
|---|---|---|---|---|
| | | (uM) | | |
| FLJ16517 - Pranlukast | | 0 | 23.8 | 47.5 |
| compound | 0 | 0.0 | 0.0 | 0.0 |
| concentration | 1 | 0.0 | 0.3 | 0.4 |
| (uM) | 10 | 0.0 | 4.2 | 5.2 |
| | 100 | 0.0 | 81.6 | 54.8 |
| | 250 | 0.1 | 42.3 | 46.0 |

Minerals (+)
measured Mass Range: m/z = 481.5-483

TABLE 193A

| | | protein concentration | | |
|---|---|---|---|---|
| | | (uM) | | |
| FLJ16517 - Luteolin | | 0 | 23.8 | 47.5 |
| compound | 0 | 0.0 | 0.0 | 0.0 |
| concentration | 1 | 0.0 | 0.0 | 0.0 |
| (uM) | 10 | 0.1 | 0.0 | 0.1 |
| | 100 | 0.2 | 0.1 | 0.9 |
| | 250 | 0.2 | 0.2 | 0.7 |

Minerals (−)
measured Mass Range: m/z = 286.2-287.7

TABLE 193B

| | | protein concentration | | |
|---|---|---|---|---|
| | | (uM) | | |
| FLJ16517 - Luteolin | | 0 | 23.8 | 47.5 |
| compound | 0 | 0.0 | 0.0 | 0.0 |
| concentration | 1 | −0.2 | 0.1 | 0.0 |
| (uM) | 10 | −0.2 | 0.4 | 0.6 |
| | 100 | −0.1 | 24.7 | 23.5 |
| | 250 | 0.0 | 33.8 | 37.1 |

Minerals (+)
measured Mass Range: m/z = 286.2-287.7

TABLE 194A

| | | protein concentration | | |
|---|---|---|---|---|
| FLJ26591 - | | (uM) | | |
| Pyrithyldione | | 0 | 23.8 | 47.5 |
| compound | 0 | 0.0 | 0.0 | 0.0 |
| concentration | 1 | −0.4 | −0.3 | −0.4 |
| (uM) | 10 | −0.2 | −0.1 | −0.2 |
| | 100 | 0.1 | 0.5 | 1.1 |
| | 250 | 2.6 | 2.0 | −0.1 |

Minerals (−)
measured Mass Range: m/z = 167.2-168.7

TABLE 194B

| | | protein concentration | | |
|---|---|---|---|---|
| FLJ26591 - | | (uM) | | |
| Pyrithyldione | | 0 | 23.8 | 47.5 |
| compound | 0 | 0.0 | 0.0 | 0.0 |
| concentration | 1 | −0.3 | −0.4 | −0.2 |
| (uM) | 10 | −0.2 | 0.1 | 0.1 |
| | 100 | 7.0 | 13.7 | 13.3 |
| | 250 | 8.5 | 34.0 | 42.1 |

Minerals (+)
measured Mass Range: m/z = 167.2-168.7

TABLE 195A

| | | protein concentration | | |
|---|---|---|---|---|
| | | (uM) | | |
| FLJ90480 - Buformin | | 0 | 23.8 | 47.5 |
| compound | 0 | 0.0 | 0.0 | 0.0 |
| concentration | 1 | 0.2 | 0.1 | −0.3 |
| (uM) | 10 | 0.1 | 0.0 | 0.1 |
| | 100 | 1.0 | 4.5 | 3.9 |
| | 250 | 6.3 | 14.6 | 29.5 |

Minerals (−)
measured Mass Range: m/z = 157.2-158.7

TABLE 195B

| | | protein concentration | | |
|---|---|---|---|---|
| | | (uM) | | |
| FLJ90480 - Buformin | | 0 | 23.8 | 47.5 |
| compound | 0 | 0.0 | 0.0 | 0.0 |
| concentration | 1 | 0.1 | −0.1 | 0.1 |
| (uM) | 10 | 0.3 | 0.3 | 0.4 |
| | 100 | 1.8 | 14.2 | 6.8 |
| | 250 | 23.0 | 16.9 | 26.1 |

Minerals (+)
measured Mass Range: m/z = 157.2-158.7

TABLE 196A

| | | protein concentration | | |
|---|---|---|---|---|
| FLJ90480 - 6- | | (uM) | | |
| Furfurylaminopurine | | 0 | 23.8 | 47.5 |
| compound | 0 | 0.0 | 0.0 | 0.0 |
| concentration | 1 | 0.1 | 0.0 | 0.1 |
| (uM) | 10 | 0.2 | 0.0 | 0.4 |
| | 100 | 1.9 | 3.0 | 5.4 |
| | 250 | 4.3 | 14.8 | 12.9 |

Minerals (−)
measured Mass Range: m/z = 215.2-216.7

TABLE 196B

| | | protein concentration | | |
|---|---|---|---|---|
| FLJ90480 - 6- | | (uM) | | |
| Furfurylaminopurine | | 0 | 23.8 | 47.5 |
| compound | 0 | 0.0 | 0.0 | 0.0 |
| concentration | 1 | 0.0 | −0.1 | −0.2 |
| (uM) | 10 | 0.0 | 0.1 | −0.2 |
| | 100 | 1.9 | 1.7 | 5.1 |
| | 250 | 0.3 | 7.4 | 14.6 |

Minerals (+)
measured Mass Range: m/z = 215.2-216.7

TABLE 197A

| FLJ90480 - Nitrarine | | protein concentration | | |
|---|---|---|---|---|
| (Nitrarine | | (uM) | | |
| dihydrochloride) | | 0 | 23.8 | 47.5 |
| compound | 0 | 0.0 | 0.0 | 0.0 |
| concentration | 1 | 0.0 | 0.0 | 0.0 |
| (uM) | 10 | 0.4 | 0.4 | 0.6 |
| | 100 | 3.0 | 6.0 | 12.5 |
| | 250 | 8.7 | 23.0 | 34.4 |

Minerals (−)
measured Mass Range: m/z = 307.4-308.9

TABLE 197B

| FLJ90480 - Nitrarine | | protein concentration | | |
|---|---|---|---|---|
| (Nitrarine | | (uM) | | |
| dihydrochloride) | | 0 | 23.8 | 47.5 |
| compound | 0 | 0.0 | 0.0 | 0.0 |
| concentration | 1 | −0.2 | 0.0 | 0.0 |
| (uM) | 10 | 0.1 | 0.5 | 1.6 |
| | 100 | 3.3 | 11.5 | 15.8 |
| | 250 | 4.7 | 6.8 | 15.6 |

Minerals (+)
measured Mass Range: m/z = 307.4-308.9

TABLE 198

| | | protein concentration | | |
|---|---|---|---|---|
| FLJ90480 - Pempidine | | (uM) | | |
| (Pempidine tartrate) | | 0 | 23.8 | 47.5 |
| compound | 0 | 0.0 | 0.0 | 0.0 |
| concentration | 1 | 0.0 | 0.1 | 0.0 |
| (uM) | 10 | 0.2 | 0.1 | 0.1 |
| | 100 | 1.7 | 13.8 | 14.8 |
| | 250 | 44.8 | 2.0 | 8.5 |

Minerals (+)
measured Mass Range: m/z = 155.3-156.8

TABLE 199A

| | | protein concentration | | |
|---|---|---|---|---|
| | | (uM) | | |
| FLJ43067 - Viloxazine | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.1 | 0.2 |
| | 100 | 4.9 | 9.3 | 15.1 |
| | 250 | 14.4 | 27.7 | 34.7 |

Minerals (−)
measured Mass Range: m/z = 237.3-238.8

TABLE 199B

| | | protein concentration | | |
|---|---|---|---|---|
| | | (uM) | | |
| FLJ43067 - Viloxazine | | 0 | 23.8 | 47.5 |
| compound | 0 | 0.0 | 0.0 | 0.0 |
| concentration | 1 | 0.8 | 0.2 | −2.8 |
| (uM) | 10 | 0.0 | 0.8 | −2.3 |
| | 100 | 4.1 | 13.1 | 14.4 |
| | 250 | 25.6 | 43.9 | 43.1 |

Minerals (+)
measured Mass Range: m/z = 237.3-238.8

TABLE 200A

| | | protein concentration | | |
|---|---|---|---|---|
| | | (uM) | | |
| FLJ25460 - Cefazolin | | 0 | 11.9 | 23.8 |
| compound | 0 | 0.0 | 0.0 | 0.0 |
| concentration | 1 | −3.6 | 1.0 | 1.3 |
| (uM) | 10 | 2.3 | 0.4 | 1.8 |
| | 100 | −0.9 | 3.0 | 4.1 |
| | 250 | 8.2 | 22.4 | 23.5 |

Minerals (−)
measured Mass Range: m/z = 453.5-455

TABLE 200B

| | | protein concentration | | |
|---|---|---|---|---|
| | | (uM) | | |
| FLJ25460 - Cefazolin | | 0 | 11.9 | 23.8 |
| compound | 0 | 0.0 | 0.0 | 0.0 |
| concentration | 1 | −6.9 | 0.5 | −0.5 |
| (uM) | 10 | 0.4 | 2.3 | 2.7 |
| | 100 | −1.6 | 3.9 | 8.5 |
| | 250 | 3.6 | 28.7 | 22.0 |

Minerals (+)
measured Mass Range: m/z = 453.5-455

| | | TABL3 201A | | |
|---|---|---|---|---|
| | | protein concentration | | |
| | | (uM) | | |
| FLJ25460 - Fenbufen | | 0 | 11.9 | 23.8 |
| compound | 0 | 0.0 | 0.0 | 0.0 |
| concentration | 1 | −0.6 | −1.4 | −1.3 |
| (uM) | 10 | −1.3 | −1.2 | −1.2 |
| | 100 | −0.9 | 0.7 | −0.2 |
| | 250 | −1.1 | −0.2 | −1.8 |

Minerals (−)
measured Mass Range: m/z = 254.3-255.8

TABLE 201B

| | | protein concentration | | |
|---|---|---|---|---|
| | | (uM) | | |
| FLJ25460 - Fenbufen | | 0 | 11.9 | 23.8 |
| compound | 0 | 0.0 | 0.0 | 0.0 |
| concentration | 1 | −2.2 | −1.0 | −0.2 |
| (uM) | 10 | −3.1 | −0.9 | −0.9 |
| | 100 | −3.4 | 0.0 | 4.0 |
| | 250 | 3.2 | 6.5 | 42.4 |

Minerals (+)
measured Mass Range: m/z = 254.3-255.8

TABLE 202A

| | | protein concentration | | |
|---|---|---|---|---|
| FLJ25460 - | | (uM) | | |
| Ketoprofen | | 0 | 11.9 | 23.8 |
| compound | 0 | 0.0 | 0.0 | 0.0 |
| concentration | 1 | −0.2 | 0.7 | −0.3 |
| (uM) | 10 | −0.5 | 0.0 | −0.2 |
| | 100 | 0.9 | 1.0 | 3.9 |
| | 250 | 2.5 | 7.1 | 9.8 |

Minerals (−)
measured Mass Range: m/z = 254.3-255.8

TABLE 202B

| | | protein concentration | | |
|---|---|---|---|---|
| FLJ25460 - | | (uM) | | |
| Ketoprofen | | 0 | 11.9 | 23.8 |
| compound | 0 | 0.0 | 0.0 | 0.0 |
| concentration | 1 | −0.6 | −0.3 | 0.0 |
| (uM) | 10 | −0.4 | −0.4 | 0.4 |
| | 100 | 2.0 | 2.9 | 7.2 |
| | 250 | 7.0 | 4.6 | 33.3 |

Minerals (+)
measured Mass Range: m/z = 254.3-255.8

TABLE 203A

| | | protein concentration | | |
|---|---|---|---|---|
| | | (uM) | | |
| FLJ25460 - Colchicine | | 0 | 11.9 | 23.8 |
| compound | 0 | 0.0 | 0.0 | 0.0 |
| concentration | 1 | 0.0 | 0.2 | 0.1 |
| (uM) | 10 | 0.7 | 0.9 | 0.4 |
| | 100 | 8.7 | 9.6 | 7.9 |
| | 250 | 23.9 | 24.1 | 17.5 |

Minerals (−)
measured Mass Range: m/z = 399.4-400.9

TABLE 203B

| | | protein concentration | | |
|---|---|---|---|---|
| | | (uM) | | |
| FLJ25460 - Colchicine | | 0 | 11.9 | 23.8 |
| compound | 0 | 0.0 | 0.0 | 0.0 |
| concentration | 1 | -0.1 | 0.1 | 0.2 |
| (uM) | 10 | 0.8 | 1.1 | 1.0 |
| | 100 | 11.4 | 14.3 | 9.5 |
| | 250 | 25.2 | 30.8 | 28.5 |

Minerals (+)
measured Mass Range: m/z = 399.4-400.9

TABLE 204A

| | | protein concentration (uM) | | |
|---|---|---|---|---|
| FLJ25460 - Doxycycline | | 0 | 11.9 | 23.8 |
| compound | 0 | 0.0 | 0.0 | 0.0 |
| concentration | 1 | 0.0 | 0.1 | 0.0 |
| (uM) | 10 | 0.1 | 0.2 | 0.2 |
| | 100 | 2.4 | 2.4 | 4.1 |
| | 250 | 4.9 | 7.8 | 7.6 |

Minerals (−)
measured Mass Range: m/z = 444.4-445.9

TABLE 204B

| | | protein concentration (uM) | | |
|---|---|---|---|---|
| FLJ25460 - Doxycycline | | 0 | 11.9 | 23.8 |
| compound | 0 | 0.0 | 0.0 | 0.0 |
| concentration | 1 | 0.0 | 0.1 | 0.2 |
| (uM) | 10 | 0.3 | 0.5 | 0.5 |
| | 100 | 5.2 | 7.8 | 7.3 |
| | 250 | 12.8 | 16.6 | 18.0 |

Minerals (+)
measured Mass Range: m/z = 444.4-445.9

TABLE 205

| | | protein concentration (uM) | | |
|---|---|---|---|---|
| FLJ25460 - Gabapentin | | 0 | 23.8 | 47.5 |
| compound | 0 | 0.0 | 0.0 | 0.0 |
| concentration | 1 | −16.8 | −3.4 | −1.7 |
| (uM) | 10 | −4.4 | 8.4 | −1.8 |
| | 100 | 11.8 | 33.8 | 37.3 |
| | 250 | 20.4 | 33.4 | 22.0 |

Minerals (+)
measured Mass Range: m/z = 171.2-172.7

TABLE 206A

| | | protein concentration (uM) | | |
|---|---|---|---|---|
| FLJ25460 - Lidoflazine | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 1.4 | 1.1 | 1.4 |
| | 250 | 4.4 | 2.4 | 3.6 |

Minerals (−)
measured Mass Range: m/z = 491.6-493.1

TABLE 206B

| | | protein concentration (uM) | | |
|---|---|---|---|---|
| FLJ25460 - Lidoflazine | | 0 | 11.9 | 23.8 |
| compound | 0 | 0.0 | 0.0 | 0.0 |
| concentration | 1 | 0.0 | 0.0 | 0.0 |
| (uM) | 10 | 0.0 | 0.1 | 0.1 |
| | 100 | 2.3 | 1.5 | 1.0 |
| | 250 | 2.6 | 3.6 | 5.6 |

Minerals (+)
measured Mass Range: m/z = 491.6-493.1

TABLE 207A

| | | protein concentration (uM) | | |
|---|---|---|---|---|
| FLJ25460 - Probenecid | | 0 | 11.9 | 23.8 |
| compound | 0 | 0.0 | 0.0 | 0.0 |
| concentration | 1 | 0.1 | 0.0 | 0.1 |
| (uM) | 10 | 0.8 | 0.9 | 0.7 |
| | 100 | 7.5 | 8.7 | 8.2 |
| | 250 | 20.4 | 15.5 | 21.8 |

Minerals (−)
measured Mass Range: m/z = 285.4-286.9

TABLE 207B

| | | protein concentration (uM) | | |
|---|---|---|---|---|
| FLJ25460 - Probenecid | | 0 | 11.9 | 23.8 |
| compound | 0 | 0.0 | 0.0 | 0.0 |
| concentration | 1 | −7.2 | 0.2 | −0.7 |
| (uM) | 10 | −6.0 | 1.8 | 1.3 |
| | 100 | 12.1 | 27.3 | 12.5 |
| | 250 | 41.3 | 45.4 | 45.7 |

Minerals (+)
measured Mass Range: m/z = 285.4-286.9

TABLE 208A

| | | protein concentration (uM) | | |
|---|---|---|---|---|
| FLJ26806 - Benzydamine | | 0 | 23.8 | 47.5 |
| compound | 0 | 0.0 | 0.0 | 0.0 |
| concentration | 1 | 0.1 | 0.0 | 0.0 |
| (uM) | 10 | 0.2 | 0.0 | 0.0 |
| | 100 | 2.4 | 5.4 | 4.9 |
| | 250 | 7.0 | 16.5 | 20.0 |

Minerals (−)
measured Mass Range: m/z = 309.4-310.9

TABLE 208B

| | | protein concentration (uM) | | |
|---|---|---|---|---|
| FLJ26806 - Benzydamine | | 0 | 23.8 | 47.5 |
| compound | 0 | 0.0 | 0.0 | 0.0 |
| concentration | 1 | 0.0 | 0.1 | 0.0 |
| (uM) | 10 | 0.2 | 0.2 | 0.4 |
| | 100 | 8.5 | 8.1 | 7.2 |
| | 250 | 18.8 | 19.7 | 0.3 |

Minerals (+)
measured Mass Range: m/z = 309.4-310.9

TABLE 209A

| | | protein concentration (uM) | | |
|---|---|---|---|---|
| FLJ26806 - Clenbuterol | | 0 | 23.8 | 47.5 |
| compound | 0 | 0.0 | 0.0 | 0.0 |
| concentration | 1 | 0.0 | 0.0 | 0.0 |
| (uM) | 10 | 0.2 | 0.3 | 0.3 |
| | 100 | 2.4 | 4.4 | 4.2 |
| | 250 | 6.0 | 8.3 | 7.2 |

Minerals (−)
measured Mass Range: m/z = 277.2-278.7

TABLE 209B

| | | protein concentration (uM) | | |
|---|---|---|---|---|
| FLJ26806 - Clenbuterol | | 0 | 23.8 | 47.5 |
| compound | 0 | 0.0 | 0.0 | 0.0 |
| concentration | 1 | 0.0 | 0.0 | 0.0 |
| (uM) | 10 | 0.3 | 0.3 | 0.3 |
| | 100 | 5.0 | 5.4 | 6.3 |
| | 250 | 10.5 | 17.8 | 18.3 |

Minerals (+)
measured Mass Range: m/z = 277.2-278.7

TABLE 210A

| | | protein concentration (uM) | | |
|---|---|---|---|---|
| FLJ43911 - Benzethonium | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 1.5 | 0.6 | 8.1 |
| | 250 | 3.0 | 29.5 | 26.3 |

Minerals (−)
measured Mass Range: m/z = 412.6-414.1

TABLE 210B

| | | protein concentration (uM) | | |
|---|---|---|---|---|
| FLJ43911 - Benzethonium | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.1 | 1.2 | 0.7 |
| | 250 | 1.2 | 2.7 | 5.3 |

Minerals (+)
measured Mass Range: m/z = 412.6-414.1

TABLE 211A

| | | protein concentration (uM) | | |
|---|---|---|---|---|
| FLJ43911 - Fluphenazine | | 0 | 23.8 | 47.5 |
| compound | 0 | 0.0 | 0.0 | 0.0 |
| concentration | 1 | 0.0 | 0.0 | −0.1 |
| (uM) | 10 | 0.0 | 0.0 | −0.1 |
| | 100 | 0.6 | 0.5 | 0.6 |
| | 250 | 2.1 | 1.9 | 6.6 |

Minerals (−)
measured Mass Range: m/z = 324.4-325.9

TABLE 211B

| | | protein concentration (uM) | | |
|---|---|---|---|---|
| FLJ43911 - Fluphenazine | | 0 | 23.8 | 47.5 |
| compound | 0 | 0.0 | 0.0 | 0.0 |
| concentration | 1 | 0.0 | 0.0 | 0.1 |
| (uM) | 10 | 0.0 | 0.0 | 0.0 |
| | 100 | 0.5 | 0.3 | 2.6 |
| | 250 | 1.8 | 1.7 | 2.0 |

Minerals (+)
measured Mass Range: m/z = 324.4-325.9

TABLE 212A

| | | protein concentration (uM) | | |
|---|---|---|---|---|
| FLJ43911 - GBR 12909 | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.1 |
| | 100 | 0.4 | 0.7 | 0.8 |
| | 250 | 1.8 | 2.5 | 5.9 |

Minerals (−)
measured Mass Range: m/z = 450.5-452

TABLE 212B

| | | | protein concentration (uM) | | |
|---|---|---|---|---|---|
| FLJ43911 - GBR 12909 | | | 0 | 23.8 | 47.5 |
| | compound | 0 | 0.0 | 0.0 | 0.0 |
| | concentration | 1 | 0.1 | 0.0 | −0.1 |
| | (uM) | 10 | 0.1 | 0.1 | −0.1 |
| | | 100 | 0.3 | 0.3 | 0.2 |
| | | 250 | 1.7 | 0.9 | 1.4 |

Minerals (+)
measured Mass Range: m/z = 450.5-452

TABLE 213A

| | | protein concentration (uM) | | |
|---|---|---|---|---|
| FLJ43911 - Doxazosin | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.2 | 0.9 | 0.8 |
| | 250 | 0.2 | 2.5 | 2.6 |

Minerals (−)
measured Mass Range: m/z = 451.5-453

TABLE 213B

| | | protein concentration (uM) | | |
|---|---|---|---|---|
| FLJ43911 - Doxazosin | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.1 | 0.4 | 0.1 |
| | 250 | 0.1 | 0.4 | 0.5 |

Minerals (+)
measured Mass Range: m/z = 451.5-453

TABLE 214A

| | | protein concentration (uM) | | |
|---|---|---|---|---|
| FLJ43911 - Procaine | | 0 | 23.8 | 47.5 |
| compound | 0 | 0.0 | 0.0 | 0.0 |
| concentration | 1 | 0.6 | 1.6 | 2.3 |
| (uM) | 10 | 0.2 | 0.6 | 2.5 |
| | 100 | 11.2 | 12.7 | 8.4 |
| | 250 | 22.7 | 17.1 | 9.8 |

Minerals (−)
measured Mass Range: m/z = 236.3-237.8

TABLE 214B

| | | protein concentration (uM) | | |
|---|---|---|---|---|
| FLJ43911 - Procaine | | 0 | 23.8 | 47.5 |
| compound | 0 | 0.0 | 0.0 | 0.0 |
| concentration | 1 | −1.6 | 2.7 | 1.3 |
| (uM) | 10 | −0.9 | 4.2 | 6.5 |
| | 100 | 5.0 | 6.8 | 6.3 |
| | 250 | −1.1 | 15.0 | 5.8 |

Minerals (+)
measured Mass Range: m/z = 236.3-237.8

TABLE 215A

| | | protein concentration (uM) | | |
|---|---|---|---|---|
| FLJ43911 - Quinacrine | | 0 | 23.8 | 47.5 |
| compound | 0 | 0.0 | 0.0 | 0.0 |
| concentration | 1 | 0.0 | 0.0 | 0.2 |
| (uM) | 10 | 0.0 | 0.1 | 0.2 |
| | 100 | 3.3 | 1.5 | 3.3 |
| | 250 | 3.0 | 5.6 | 2.2 |

Minerals (−)
measured Mass Range: m/z = 399.9-401.4

TABLE 215B

| | | protein concentration (uM) | | |
|---|---|---|---|---|
| FLJ43911 - Quinacrine | | 0 | 23.8 | 47.5 |
| compound | 0 | 0.0 | 0.0 | 0.0 |
| concentration | 1 | −0.5 | −0.2 | −0.1 |
| (uM) | 10 | −0.5 | −0.1 | 0.0 |
| | 100 | 0.8 | 1.7 | 0.9 |
| | 250 | 1.5 | 3.4 | 2.7 |

Minerals (+)
measured Mass Range: m/z = 399.9-401.4

TABLE 216A

| FLJ44715 - | | protein concentration (uM) | | |
|---|---|---|---|---|
| Azithromycin | | 0 | 23.8 | 47.5 |
| compound | 0 | 0.0 | 0.0 | 0.0 |
| concentration | 1 | 0.1 | 0.2 | −0.8 |
| (uM) | 10 | 1.1 | 1.5 | −0.7 |
| | 100 | 7.4 | 15.6 | 14.7 |
| | 250 | 25.9 | 34.5 | 13.7 |

Minerals (−)
measured Mass Range: m/z = 749-750.5

TABLE 216B

| FLJ44715 - | | protein concentration (uM) | | |
|---|---|---|---|---|
| Azithromycin | | 0 | 23.8 | 47.5 |
| compound | 0 | 0.0 | 0.0 | 0.0 |
| concentration | 1 | 0.0 | −0.1 | −0.1 |
| (uM) | 10 | 0.9 | 0.8 | 1.4 |
| | 100 | 9.5 | 13.1 | 18.6 |
| | 250 | 8.0 | 18.5 | 40.6 |

Minerals (+)
measured Mass Range: m/z = 749-750.5

TABLE 217

| | | protein concentration (uM) | | |
|---|---|---|---|---|
| FLJ44715 - Colistin | | 0 | 23.8 | 47.5 |
| compound | 0 | 0.0 | 0.0 | 0.0 |
| concentration | 1 | −0.1 | −0.2 | 2.5 |
| (uM) | 10 | −0.2 | 1.0 | 2.9 |
| | 100 | 1.7 | 17.2 | 17.1 |
| | 250 | 31.6 | 59.3 | 59.9 |

Minerals (+)
measured Mass Range: m/z = 1155.5-1157

TABLE 218A

| | | protein concentration (uM) | | |
|---|---|---|---|---|
| FLJ90031 - Protriptyline | | 0 | 23.8 | 47.5 |
| compound | 0 | 0.0 | 0.0 | 0.0 |
| concentration | 1 | −0.1 | 0.0 | 0.0 |
| (uM) | 10 | −0.1 | 0.0 | 0.2 |
| | 100 | 0.4 | 0.8 | 2.2 |
| | 250 | 1.1 | 1.7 | 2.8 |

Minerals (−)
measured Mass Range: m/z = 263.4-264.9

TABLE 218B

| | | protein concentration (uM) | | |
|---|---|---|---|---|
| FLJ90031 - Protriptyline | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.6 | 0.9 | 1.3 |
| | 250 | 3.9 | 0.7 | 3.7 |

Minerals (+)
measured Mass Range: m/z = 263.4-264.9

TABLE 219A

| | | protein concentration (uM) | | |
|---|---|---|---|---|
| FLJ90031 - Maprotiline | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.6 | 0.6 | 1.1 |
| | 250 | 2.7 | 1.7 | 6.1 |

Minerals (−)
measured Mass Range: m/z = 277.4-278.9

TABLE 219B

| | | protein concentration (uM) | | |
|---|---|---|---|---|
| FLJ90031 - Maprotiline | | 0 | 23.8 | 47.5 |
| compound | 0 | 0.0 | 0.0 | 0.0 |
| concentration | 1 | 0.0 | 0.0 | 0.0 |
| (uM) | 10 | 0.0 | 0.0 | 0.0 |
| | 100 | 0.6 | 0.8 | 0.8 |
| | 250 | 0.7 | 3.9 | 3.5 |

Minerals (+)
measured Mass Range: m/z = 277.4-278.9

Accordingly, the proteins that shows interaction were proved one of the target proteins of the pairs of the compounds corresponding thereof. Therefore, a new drug can be screened by making the protein interact with screening candidate substances. Specifically, a new drug can be screened by, for example, constructing a system which detects the interaction between the protein and a candidate substance according to the method of Reference Example 4.

### Example 2

### Analysis of Interaction Between Expressed Protein and Compound (2)

Expression and purification of various proteins were performed according to the methods of Reference Examples 1 to 3, and the interactions between the various proteins and various compounds were analyzed according to the method of Reference Example 5. The binding strength (K_{d} value) relating to the pairs of various proteins and various compounds that showed interaction are shown in the following Tables 220-1 and 220-2.

TABLE 220-1

| SEQ ID NO: | FLJ | compound | Biacore KD(M) |
|---|---|---|---|
| 1 | FLJ21182 | Diphenidol | 0.000453 |
| 1 | FLJ21182 | Pimethixene | 0.0018 |
| 1 | FLJ21182 | Alimemazine | 0.00011 |
| 1 | FLJ21182 | Boldine | 0.000168 |
| 1 | FLJ21182 | Clofilium | 0.000507 |
| 1 | FLJ21182 | Paroxetine | 0.000929 |
| 9 | FLJ43792 | Terguride | 0.0000262 |
| 10 | FLJ38127 | Eburnamonine | 0.0143 |
| 14 | FLJ90682 | Bupivacaine | 0.00358 |
| 20 | FLJ26144 | Pranlukast | 0.00000275 |
| 21 | FLJ26374 | Pranlukast | 0.00106 |
| 22 | FLJ26371 | Clemizole | 0.0016 |
| 22 | FLJ26371 | Harmol | 0.000275 |
| 22 | FLJ26371 | Piperlongumine | 0.0018 |
| 22 | FLJ26371 | Propranolol | 0.00841 |
| 23 | FLJ45688 | Cyclobenzaprine | 0.000118 |
| 23 | FLJ45688 | Flupentixol | 0.000586 |
| 23 | FLJ45688 | Guanfacine | 0.000262 |
| 23 | FLJ45688 | Maprotiline | 0.0128 |
| 23 | FLJ45688 | Perhexiline | 0.0131 |
| 23 | FLJ45688 | Clenbuterol | 0.00987 |
| 23 | FLJ45688 | Etodolac | 0.0126 |
| 25 | FLJ26267 | Metergotamine | 0.017 |
| 25 | FLJ26267 | Methoxamine | 0.0046 |
| 25 | FLJ26267 | Paroxetine | 0.00187 |
| 25 | FLJ26267 | Dizocilpine | 0.000482 |
| 25 | FLJ26267 | 3-Hydroxykynurenine | 0.00571 |
| 26 | FLJ26062 | Fenoprofen | 0.00173 |
| 27 | FLJ22936 | Acenocoumarol | 0.00466 |
| 27 | FLJ22936 | Budesonide | 0.00997 |
| 27 | FLJ22936 | Diclofenac | 0.0000733 |
| 27 | FLJ22936 | Diperodon | 0.0012 |
| 27 | FLJ22936 | DO 897/99 | 0.000402 |
| 27 | FLJ22936 | Nimesulide | 0.000161 |
| 27 | FLJ22936 | Thioproperasine | 0.00019 |
| 27 | FLJ22936 | Sarpogrelate | 0.01 |
| 28 | FLJ43223 | Acetyisalicylsalicylic acid | 0.000181 |

TABLE 220-2

| | | | |
|---|---|---|---|
| 29 | FLJ26102 | Buspirone | 0.00142 |
| 30 | FLJ25218 | Dopamine | 0.0000107 |
| 30 | FLJ25218 | Alpha-methyl-5-hydroxytryptamine | 0.00457 |
| 32 | FLJ25918 | Tranilast | 0.000738 |
| 34 | RGNpc017 | Domperidone | 0.000112 |
| 34 | RGNpc017 | Fluphenazine | 0.00508 |
| 34 | RGNpc017 | Trifluoperazine | 0.00719 |
| 34 | RGNpc017 | Clinofibrate | 0.000774 |
| 34 | RGNpc017 | Acetohexamide | 2.48E−05 |
| 35 | FLJ40377 | Doxazosin | 0.000714 |
| 35 | FLJ40377 | Pranlukast | 0.000013 |
| 36 | FLJ25845 | Acetopromazine | 0.00181 |
| 36 | FLJ25845 | Perhexiline | 0.00901 |
| 36 | FLJ25845 | Terconazole | 0.00161 |
| 36 | FLJ25845 | Amoxapine | 0.00128 |
| 36 | FLJ25845 | Cephaeline | 0.0132 |
| 36 | FLJ25845 | Domperidone | 0.00842 |
| 36 | FLJ25845 | Moxalactam | 0.000643 |
| 40 | FLJ90364 | Pirenzepine | 0.00014 |
| 43 | FLJ46896 | Hydroxytacrine (R,S) | 0.0107 |
| 43 | FLJ46896 | Metaproterenol | 0.00519 |
| 45 | FLJ90345 | Norharman | 0.00789 |
| 46 | FLJ26550 | Nitrarine | 0.000336 |
| 49 | FLJ45115 | Josamycin | 0.00183 |
| 51 | FLJ37995 | Diclofenamide | 0.000367 |
| 51 | FLJ37995 | Benzthiazide | 0.0012 |
| 52 | FLJ26058 | Hydroxychloroquine | 0.00018 |
| 53 | FLJ46369 | Domperidone | 0.00885 |
| 53 | FLJ46369 | Doxazosin | 0.0126 |
| 53 | FLJ46369 | Syrosingopine | 0.013 |
| 54 | FLJ16517 | Domperidone | 0.0000874 |
| 57 | FLJ90480 | Nitrarine | 0.000331 |
| 59 | FLJ25460 | Ketoprofen | 0.000037 |
| 59 | FLJ25460 | Gabapentin | 0.00011 |
| 59 | FLJ25460 | Lidoflazine | 0.000562 |
| 60 | FLJ26806 | Benzydamine | 0.00901 |
| 61 | FLJ43911 | Quinacrine | 0.0000808 |
| 63 | FLJ90031 | Protriptyline | 0.00948 |
| 63 | FLJ90031 | Maprotiline | 0.00142 |

Accordingly, the proteins that shows interaction were proved one of the target proteins of the pairs of the compounds corresponding thereof. Therefore, a new drug can be screened by making the protein interact with screening candidate substances. Specifically, a new drug can be screened by, for example, constructing a system which detects the interaction between the protein and a candidate substance according to the method of Reference Example 5.

### INDUSTRIAL APPLICABILITY

The target proteins and target genes of the present invention are useful for enable the development of bioactive substances, for example, drug discovery and the like. The screening methods of the present invention and the derivative production method of the present invention are useful for the development of prophylactic or therapeutic agents for various diseases or conditions, and investigational reagents for the diseases or the conditions, and the like. The regulators and derivatives of the present invention are useful for the prophylaxis and treatment of various diseases or conditions, and the development of investigational reagents for the diseases or the conditions, and the like. The complexes and kits of the present invention are useful for the screening methods of the present invention, the derivative production methods of the present invention and the like. The determination methods and determination kits of the present invention are useful for the evaluation of the onset or likelihood of onset of various diseases or conditions in animals, and the evaluation of the susceptibility of animals to bioactive substances and the like.

This application is based on a patent application No. 2007-040541 filed on Feb. 21, 2007 in Japan, the contents of which are incorporated in full herein by this reference.

1. A method for screening a substance capable of regulating an action associated with a bioactive substance X, which comprises determining whether or not a test substance is capable of regulating the expression or function of a target protein Y or a gene that encodes the protein, wherein the combination of the bioactive substance X and the target protein Y is any of the following (a1) to (a192):
(a1) a combination of trimethylcolchicic acid and a protein comprising the amino acid sequence shown by SEQ ID NO:1 or SEQ ID NO:2 or a protein homologous thereto or a variant thereof;
(a2) a combination of acenocoumarol and a protein comprising the amino acid sequence shown by SEQ ID NO:27 or a protein homologous thereto or a variant thereof;
(a3) a combination of paracetamol and a protein comprising the amino acid sequence shown by SEQ ID NO:3 or a protein homologous thereto or a variant thereof;
(a4) a combination of acetohexamide and a protein comprising the amino acid sequence shown by SEQ ID NO:23, SEQ ID NO:24 or SEQ ID NO:34 or a protein homologous thereto or a variant thereof;
(a5) a combination of acetopromazine and a protein comprising the amino acid sequence shown by SEQ ID NO:36 or a protein homologous thereto or a variant thereof;
(a6) a combination of actinomycin D and a protein comprising the amino acid sequence shown by SEQ ID NO:54 or a protein homologous thereto or a variant thereof;
(a7) a combination of ajmaline and a protein comprising the amino acid sequence shown by SEQ ID NO:1 or SEQ ID NO:2 or a protein homologous thereto or a variant thereof;
(a8) a combination of albendazole and a protein comprising the amino acid sequence shown by SEQ ID NO:38 or a protein homologous thereto or a variant thereof;
(a9) a combination of alfuzosin and a protein comprising the amino acid sequence shown by SEQ ID NO:35 or a protein homologous thereto or a variant thereof;
(a10) a combination of α-methyl-5-hydroxytryptamine and a protein comprising the amino acid sequence shown by SEQ ID NO:30 or a protein homologous thereto or a variant thereof;
(a11) a combination of amoxapine and a protein comprising the amino acid sequence shown by SEQ ID NO:36 or a protein homologous thereto or a variant thereof;
(a12) a combination of antipyrine and a protein comprising the amino acid sequence shown by SEQ ID NO:1 or a protein homologous thereto or a variant thereof;
(a13) a combination of azithromycin and a protein comprising the amino acid sequence shown by SEQ ID NO:62 or a protein homologous thereto or a variant thereof;
(a14) a combination of benzbromarone and a protein comprising the amino acid sequence shown by SEQ ID NO:42, SEQ ID NO:53 or SEQ ID NO:54 or a protein homologous thereto or a variant thereof;
(a15) a combination of benzethonium and a protein comprising the amino acid sequence shown by SEQ ID NO:23, SEQ ID NO:53 or SEQ ID NO:61 or a protein homologous thereto or a variant thereof;
(a16) a combination of benzydamine and a protein comprising the amino acid sequence shown by SEQ ID NO:60 or a protein homologous thereto or a variant thereof;
(a17) a combination of berberine and a protein comprising the amino acid sequence shown by SEQ ID NO:32 or a protein homologous thereto or a variant thereof;
(a18) a combination of bezafibrate and a protein comprising the amino acid sequence shown by SEQ ID NO:39 or a protein homologous thereto or a variant thereof;
(a19) a combination of bicartamide and a protein comprising the amino acid sequence shown by SEQ ID NO:53 or a protein homologous thereto or a variant thereof;
(a20) a combination of boldine and a protein comprising the amino acid sequence shown by SEQ ID NO:1 or a protein homologous thereto or a variant thereof;
(a21) a combination of bromperidol and a protein comprising the amino acid sequence shown by SEQ ID NO:33 or a protein homologous thereto or a variant thereof;
(a22) a combination of budesonide and a protein comprising the amino acid sequence shown by SEQ ID NO:27 or a protein homologous thereto or a variant thereof;
(a23) a combination of bupivacaine and a protein comprising the amino acid sequence shown by SEQ ID NO:14 or a protein homologous thereto or a variant thereof;
(a24) a combination of buspirone and a protein comprising the amino acid sequence shown by SEQ ID NO:29 or a protein homologous thereto or a variant thereof;
(a25) a combination of cefazolin and a protein comprising the amino acid sequence shown by SEQ ID NO:59 or a protein homologous thereto or a variant thereof;
(a26) a combination of celestine blue and a protein comprising the amino acid sequence shown by SEQ ID NO:1, SEQ ID NO:2, SEQ ID NO:3, SEQ ID NO:32 or SEQ ID NO:46 or a protein homologous thereto or a variant thereof;
(a27) a combination of cephaeline and a protein comprising the amino acid sequence shown by SEQ ID NO:1 or SEQ ID NO:36 or a protein homologous thereto or a variant thereof;
(a28) a combination of chlordiazepoxide and a protein comprising the amino acid sequence shown by SEQ ID NO:56 or a protein homologous thereto or a variant thereof;
(a29) a combination of chlorogenic acid and a protein comprising the amino acid sequence shown by SEQ ID NO:27 or a protein homologous thereto or a variant thereof;
(a30) a combination of chlorothiazide and a protein comprising the amino acid sequence shown by SEQ ID NO:27 or a protein homologous thereto or a variant thereof;
(a31) a combination of chromomycin A3 and a protein comprising the amino acid sequence shown by SEQ ID NO:17 or SEQ ID NO:34 or a protein homologous thereto or a variant thereof;
(a32) a combination of ciclopirox and a protein comprising the amino acid sequence shown by SEQ ID NO:1 or SEQ ID NO:3 or a protein homologous thereto or a variant thereof;
(a33) a combination of cisapride and a protein comprising the amino acid sequence shown by SEQ ID NO:31 or a protein homologous thereto or a variant thereof;
(a34) a combination of clarithromycin and a protein comprising the amino acid sequence shown by SEQ ID NO:49 or a protein homologous thereto or a variant thereof;
(a35) a combination of clemizole and a protein comprising the amino acid sequence shown by SEQ ID NO:22 or SEQ ID NO:47 or a protein homologous thereto or a variant thereof;
(a36) a combination of clenbuterol and a protein comprising the amino acid sequence shown by SEQ ID NO:23, SEQ ID NO:36 or SEQ ID NO:60 or a protein homologous thereto or a variant thereof;
(a37) a combination of clobetasone and a protein comprising the amino acid sequence shown by SEQ ID NO:35 or a protein homologous thereto or a variant thereof;
(a38) a combination of clofazimine and a protein comprising the amino acid sequence shown by SEQ ID NO:15, SEQ ID NO:37, SEQ ID NO:53 or SEQ ID NO:54 or a protein homologous thereto or a variant thereof;
(a39) a combination of clofilium and a protein comprising the amino acid sequence shown by SEQ ID NO:1 or a protein homologous thereto or a variant thereof;
(a40) a combination of clomiphene and a protein comprising the amino acid sequence shown by SEQ ID NO:23 or a protein homologous thereto or a variant thereof;
(a41) a combination of clopamide and a protein comprising the amino acid sequence shown by SEQ ID NO:23 or a protein homologous thereto or a variant thereof;
(a42) a combination of colchicine and a protein comprising the amino acid sequence shown by SEQ ID NO:59 or a protein homologous thereto or a variant thereof;
(a43) a combination of colistin and a protein comprising the amino acid sequence shown by SEQ ID NO:62 or a protein homologous thereto or a variant thereof;
(a44) a combination of conessine and a protein comprising the amino acid sequence shown by SEQ ID NO:1 or a protein homologous thereto or a variant thereof;
(a45) a combination of coniine (DL) and a protein comprising the amino acid sequence shown by SEQ ID NO:1 or SEQ ID NO:3 or a protein homologous thereto or a variant thereof;
(a46) a combination of coralyne and a protein comprising the amino acid sequence shown by SEQ ID NO:33 or a protein homologous thereto or a variant thereof;
(a47) a combination of cyclobenzaprine and a protein comprising the amino acid sequence shown by SEQ ID NO:23 or a protein homologous thereto or a variant thereof;
(a48) a combination of cyclopentolate and a protein comprising the amino acid sequence shown by SEQ ID NO:36 or a protein homologous thereto or a variant thereof;
(a49) a combination of cyclosporine A and a protein comprising the amino acid sequence shown by SEQ ID NO:50 or a protein homologous thereto or a variant thereof;
(a50) a combination of diclofenac and a protein comprising the amino acid sequence shown by SEQ ID NO:27 or a protein homologous thereto or a variant thereof;
(a51) a combination of dichlorphenamide and a protein comprising the amino acid sequence shown by SEQ ID NO:51 or a protein homologous thereto or a variant thereof;
(a52) a combination of diflunisal and a protein comprising the amino acid sequence shown by SEQ ID NO:32 or a protein homologous thereto or a variant thereof;
(a53) a combination of dihydrostreptomycin and a protein comprising the amino acid sequence shown by SEQ ID NO:19 or a protein homologous thereto or a variant thereof;
(a54) a combination of diperodon and a protein comprising the amino acid sequence shown by SEQ ID NO:27 or a protein homologous thereto or a variant thereof;
(a55) a combination of difenidol and a protein comprising the amino acid sequence shown by SEQ ID NO:1 or a protein homologous thereto or a variant thereof;
(a56) a combination of dipyridamole and a protein comprising the amino acid sequence shown by SEQ ID NO:15 or a protein homologous thereto or a variant thereof;
(a57) a combination of dizocilpine and a protein comprising the amino acid sequence shown by SEQ ID NO:25 or a protein homologous thereto or a variant thereof;
(a58) a combination of DO897/99 and a protein comprising the amino acid sequence shown by SEQ ID NO:27 or SEQ ID NO:34 or a protein homologous thereto or a variant thereof;
(a59) a combination of domperidone and a protein comprising the amino acid sequence shown by SEQ ID NO:34, SEQ ID NO:36, SEQ ID NO:53 or SEQ ID NO:54 or a protein homologous thereto or a variant thereof;
(a60) a combination of dopamine and a protein comprising the amino acid sequence shown by SEQ ID NO:30 or a protein homologous thereto or a variant thereof;
(a61) a combination of doxazocin and a protein comprising the amino acid sequence shown by SEQ ID NO:1, SEQ ID NO:35, SEQ ID NO:53 or SEQ ID NO:61 or a protein homologous thereto or a variant thereof;
(a62) a combination of doxycycline and a protein comprising the amino acid sequence shown by SEQ ID NO:59 or a protein homologous thereto or a variant thereof;
(a63) a combination of eburnamonine and a protein comprising the amino acid sequence shown by SEQ ID NO:10 or SEQ ID NO:44 or a protein homologous thereto or a variant thereof;
(a64) a combination of etodolac and a protein comprising the amino acid sequence shown by SEQ ID NO:23 or a protein homologous thereto or a variant thereof;
(a65) a combination of fenbendazole and a protein comprising the amino acid sequence shown by SEQ ID NO:22 or a protein homologous thereto or a variant thereof;
(a66) a combination of fenbufen and a protein comprising the amino acid sequence shown by SEQ ID NO:59 or a protein homologous thereto or a variant thereof;
(a67) a combination of fenoprofen and a protein comprising the amino acid sequence shown by SEQ ID NO:26 or a protein homologous thereto or a variant thereof;
(a68) a combination of flumequine and a protein comprising the amino acid sequence shown by SEQ ID NO:56 or a protein homologous thereto or a variant thereof;
(a69) a combination of flupentixol and a protein comprising the amino acid sequence shown by SEQ ID NO:23 or SEQ ID NO:34 or a protein homologous thereto or a variant thereof;
(a70) a combination of fluphenazine and a protein comprising the amino acid sequence shown by SEQ ID NO:34 or SEQ ID NO:61 or a protein homologous thereto or a variant thereof;
(a71) a combination of fluvoxamine and a protein comprising the amino acid sequence shown by SEQ ID NO:25 or a protein homologous thereto or a variant thereof;
(a72) a combination of furazolidone and a protein comprising the amino acid sequence shown by SEQ ID NO:52 or a protein homologous thereto or a variant thereof;
(a73) a combination of gabapentin and a protein comprising the amino acid sequence shown by SEQ ID NO:59 or a protein homologous thereto or a variant thereof;
(a74) a combination of GBR12909 and a protein comprising the amino acid sequence shown by SEQ ID NO:61 or a protein homologous thereto or a variant thereof;
(a75) a combination of glibenclamide and a protein comprising the amino acid sequence shown by SEQ ID NO:23 or SEQ ID NO:37 or a protein homologous thereto or a variant thereof;
(a76) a combination of glipizide and a protein comprising the amino acid sequence shown by SEQ ID NO:23 or a protein homologous thereto or a variant thereof;
(a77) a combination of gramicidin and a protein comprising the amino acid sequence shown by SEQ ID NO:53 or a protein homologous thereto or a variant thereof;
(a78) a combination of guanfacine and a protein comprising the amino acid sequence shown by SEQ ID NO:23 or a protein homologous thereto or a variant thereof;
(a79) a combination of harmol and a protein comprising the amino acid sequence shown by SEQ ID NO:22 or a protein homologous thereto or a variant thereof;
(a80) a combination of hydroflumethiazide and a protein comprising the amino acid sequence shown by SEQ ID NO:11 or a protein homologous thereto or a variant thereof;
(a81) a combination of hydroxychloroquine and a protein comprising the amino acid sequence shown by SEQ ID NO:52 or a protein homologous thereto or a variant thereof;
(a82) a combination of hydroxytacrine(R,S) and a protein comprising the amino acid sequence shown by SEQ ID NO:43 or a protein homologous thereto or a variant thereof;
(a83) a combination of ifosfamide and a protein comprising the amino acid sequence shown by SEQ ID NO:22 or a protein homologous thereto or a variant thereof;
(a84) a combination of iobenguane and a protein comprising the amino acid sequence shown by SEQ ID NO:9 or a protein homologous thereto or a variant thereof;
(a85) a combination of iproniazid and a protein comprising the amino acid sequence shown by SEQ ID NO:19 or a protein homologous thereto or a variant thereof;
(a86) a combination of isoxicam and a protein comprising the amino acid sequence shown by SEQ ID NO:23 or a protein homologous thereto or a variant thereof;
(a87) a combination of isradipine and a protein comprising the amino acid sequence shown by SEQ ID NO:24 or a protein homologous thereto or a variant thereof;
(a88) a combination of josamycin and a protein comprising the amino acid sequence shown by SEQ ID NO:49 or a protein homologous thereto or a variant thereof;
(a89) a combination of ketoprofen and a protein comprising the amino acid sequence shown by SEQ ID NO:59 or a protein homologous thereto or a variant thereof;
(a90) a combination of 3-hydroxykynurenine and a protein comprising the amino acid sequence shown by SEQ ID NO:25 or a protein homologous thereto or a variant thereof;
(a91) a combination of leuprolide and a protein comprising the amino acid sequence shown by SEQ ID NO:50 or a protein homologous thereto or a variant thereof;
(a92) a combination of L-thyroxine and a protein comprising the amino acid sequence shown by SEQ ID NO:34 or a protein homologous thereto or a variant thereof;
(a93) a combination of lidoflazine and a protein comprising the amino acid sequence shown by SEQ ID NO:59 or a protein homologous thereto or a variant thereof;
(a94) a combination of α-lobeline (−) and a protein comprising the amino acid sequence shown by SEQ ID NO:6 or a protein homologous thereto or a variant thereof;
(a95) a combination of loperamide and a protein comprising the amino acid sequence shown by SEQ ID NO:15 or SEQ ID NO:54 or a protein homologous thereto or a variant thereof;
(a96) a combination of maprotiline and a protein comprising the amino acid sequence shown by SEQ ID NO:23 or SEQ ID NO:63 or a protein homologous thereto or a variant thereof;
(a97) a combination of mebendazole and a protein comprising the amino acid sequence shown by SEQ ID NO:32 or a protein homologous thereto or a variant thereof;
(a98) a combination of meclofenamic acid and a protein comprising the amino acid sequence shown by SEQ ID NO:17 or a protein homologous thereto or a variant thereof;
(a99) a combination of metanephrine (D,L) a protein comprising the amino acid sequence shown by SEQ ID NO:52 or a protein homologous thereto or a variant thereof;
(a100) a combination of metaproterenol and a protein comprising the amino acid sequence shown by SEQ ID NO:43 or a protein homologous thereto or a variant thereof;
(a101) a combination of metergotamine and a protein comprising the amino acid sequence shown by SEQ ID NO:25 or SEQ ID NO:43 or a protein homologous thereto or a variant thereof;
(a102) a combination of methimazole and a protein comprising the amino acid sequence shown by SEQ ID NO:12 or a protein homologous thereto or a variant thereof;
(a103) a combination of methoxamine and a protein comprising the amino acid sequence shown by SEQ ID NO:25 or a protein homologous thereto or a variant thereof;
(a104) a combination of methoxy-6-harmalan and a protein comprising the amino acid sequence shown by SEQ ID NO:1 or SEQ ID NO:2 or a protein homologous thereto or a variant thereof;
(a105) a combination of mifepristone and a protein comprising the amino acid sequence shown by SEQ ID NO:42 or a protein homologous thereto or a variant thereof;
(a106) a combination of minaprine and a protein comprising the amino acid sequence shown by SEQ ID NO:2 or a protein homologous thereto or a variant thereof;
(a107) a combination of minocycline and a protein comprising the amino acid sequence shown by SEQ ID NO:36 or a protein homologous thereto or a variant thereof;
(a108) a combination of misoprostol and a protein comprising the amino acid sequence shown by SEQ ID NO:23 or a protein homologous thereto or a variant thereof;
(a109) a combination of molsidomine and a protein comprising the amino acid sequence shown by SEQ ID NO:4 or a protein homologous thereto or a variant thereof;
(a110) a combination of moroxydine and a protein comprising the amino acid sequence shown by SEQ ID NO:7 or a protein homologous thereto or a variant thereof;
(a111) a combination of moxalactam and a protein comprising the amino acid sequence shown by SEQ ID NO:36 or a protein homologous thereto or a variant thereof;
(a112) a combination of mupirocin and a protein comprising the amino acid sequence shown by SEQ ID NO:24 or a protein homologous thereto or a variant thereof;
(a113) a combination of nefopam and a protein comprising the amino acid sequence shown by SEQ ID NO:19 or a protein homologous thereto or a variant thereof;
(a114) a combination of nicardipine and a protein comprising the amino acid sequence shown by SEQ ID NO:54 or a protein homologous thereto or a variant thereof;
(a115) a combination of nimesulide and a protein comprising the amino acid sequence shown by SEQ ID NO:27 or a protein homologous thereto or a variant thereof;
(a116) a combination of norharman and a protein comprising the amino acid sequence shown by SEQ ID NO:45 or a protein homologous thereto or a variant thereof;
(a117) a combination of oxytocin and a protein comprising the amino acid sequence shown by SEQ ID NO:49 or a protein homologous thereto or a variant thereof;
(a118) a combination of paroxetine and a protein comprising the amino acid sequence shown by SEQ ID NO:1 or SEQ ID NO:25 or a protein homologous thereto or a variant thereof;
(a119) a combination of perhexiline and a protein comprising the amino acid sequence shown by SEQ ID NO:23 or SEQ ID NO:36 or a protein homologous thereto or a variant thereof;
(a120) a combination of phenformin and a protein comprising the amino acid sequence shown by SEQ ID NO:36 or a protein homologous thereto or a variant thereof;
(a121) a combination of pimethixene and a protein comprising the amino acid sequence shown by SEQ ID NO:1 or a protein homologous thereto or a variant thereof;
(a122) a combination of piperlongumine and a protein comprising the amino acid sequence shown by SEQ ID NO:22 or a protein homologous thereto or a variant thereof;
(a123) a combination of pirenzepine and a protein comprising the amino acid sequence shown by SEQ ID NO:40 or a protein homologous thereto or a variant thereof;
(a124) a combination of probenecid and a protein comprising the amino acid sequence shown by SEQ ID NO:23 or SEQ ID NO:59 or a protein homologous thereto or a variant thereof;
(a125) a combination of procaine and a protein comprising the amino acid sequence shown by SEQ ID NO:61 or a protein homologous thereto or a variant thereof;
(a126) a combination of propranolol and a protein comprising the amino acid sequence shown by SEQ ID NO:22 or a protein homologous thereto or a variant thereof;
(a127) a combination of protriptyline and a protein comprising the amino acid sequence shown by SEQ ID NO:63 or a protein homologous thereto or a variant thereof;
(a128) a combination of pyrilamine and a protein comprising the amino acid sequence shown by SEQ ID NO:36 or SEQ ID NO:45 or a protein homologous thereto or a variant thereof;
(a129) a combination of quercetin and a protein comprising the amino acid sequence shown by SEQ ID NO:20 or SEQ ID NO:54 or a protein homologous thereto or a variant thereof;
(a130) a combination of quinacrine and a protein comprising the amino acid sequence shown by SEQ ID NO:61 or a protein homologous thereto or a variant thereof;
(a131) a combination of quinine and a protein comprising the amino acid sequence shown by SEQ ID NO:1 or SEQ ID NO:10 or a protein homologous thereto or a variant thereof;
(a132) a combination of rescinnamine and a protein comprising the amino acid sequence shown by SEQ ID NO:41 or SEQ ID NO:53 or a protein homologous thereto or a variant thereof;
(a133) a combination of risperidone and a protein comprising the amino acid sequence shown by SEQ ID NO:13 or SEQ ID NO:35 or a protein homologous thereto or a variant thereof;
(a134) a combination of ritodrine and a protein comprising the amino acid sequence shown by SEQ ID NO:1 or SEQ ID NO:2 or a protein homologous thereto or a variant thereof;
(a135) a combination of saquinavir and a protein comprising the amino acid sequence shown by SEQ ID NO:17 or SEQ ID NO:53 or a protein homologous thereto or a variant thereof;
(a136) a combination of scoulerine and a protein comprising the amino acid sequence shown by SEQ ID NO:2 or a protein homologous thereto or a variant thereof;
(a137) a combination of sulfadimethoxine and a protein comprising the amino acid sequence shown by SEQ ID NO:1 or a protein homologous thereto or a variant thereof;
(a138) a combination of sulfaphenazole and a protein comprising the amino acid sequence shown by SEQ ID NO:23 or a protein homologous thereto or a variant thereof;
(a139) a combination of syrosingopine and a protein comprising the amino acid sequence shown by SEQ ID NO:53 or a protein homologous thereto or a variant thereof;
(a140) a combination of tamoxifen and a protein comprising the amino acid sequence shown by SEQ ID NO:3 or a protein homologous thereto or a variant thereof;
(a141) a combination of terconazole and a protein comprising the amino acid sequence shown by SEQ ID NO:36 or a protein homologous thereto or a variant thereof;
(a142) a combination of thioproperazine and a protein comprising the amino acid sequence shown by SEQ ID NO:23 or SEQ ID NO:27 or a protein homologous thereto or a variant thereof;
(a143) a combination of thiothixene(cis) and a protein comprising the amino acid sequence shown by SEQ ID NO:23 or a protein homologous thereto or a variant thereof;
(a144) a combination of tobramycin and a protein comprising the amino acid sequence shown by SEQ ID NO:36 or a protein homologous thereto or a variant thereof;
(a145) a combination of tolbutamide and a protein comprising the amino acid sequence shown by SEQ ID NO:23 or a protein homologous thereto or a variant thereof;
(a146) a combination of trifluoperazine and a protein comprising the amino acid sequence shown by SEQ ID NO:34 or a protein homologous thereto or a variant thereof;
(a147) a combination of trimetazidine and a protein comprising the amino acid sequence shown by SEQ ID NO:5 or a protein homologous thereto or a variant thereof;
(a148) a combination of viloxazine and a protein comprising the amino acid sequence shown by SEQ ID NO:58 or a protein homologous thereto or a variant thereof;
(a149) a combination of xylazine and a protein comprising the amino acid sequence shown by SEQ ID NO:8 or a protein homologous thereto or a variant thereof;
(a150) a combination of acetylsalicylsalicylic acid and a protein comprising the amino acid sequence shown by SEQ ID NO:28 or a protein homologous thereto or a variant thereof;
(a151) a combination of nimetazepam and a protein comprising the amino acid sequence shown by SEQ ID NO:25 or a protein homologous thereto or a variant thereof;
(a152) a combination of clobazam and a protein comprising the amino acid sequence shown by SEQ ID NO:48 or a protein homologous thereto or a variant thereof;
(a153) a combination of alimemazine and a protein comprising the amino acid sequence shown by SEQ ID NO:1 or SEQ ID NO:2 or a protein homologous thereto or a variant thereof;
(a154) a combination of tranilast and a protein comprising the amino acid sequence shown by SEQ ID NO:32 or a protein homologous thereto or a variant thereof;
(a155) a combination of ebastine and a protein comprising the amino acid sequence shown by SEQ ID NO:54 or a protein homologous thereto or a variant thereof;
(a156) a combination of pranlukast and a protein comprising the amino acid sequence shown by SEQ ID NO:18, SEQ ID NO:20, SEQ ID NO:21, SEQ ID NO:35, SEQ ID NO:42, SEQ ID NO:53 or SEQ ID NO:54 or a protein homologous thereto or a variant thereof;
(a157) a combination of methyclothiazide and a protein comprising the amino acid sequence shown by SEQ ID NO:16 or SEQ ID NO:23 or a protein homologous thereto or a variant thereof;
(a158) a combination of alacepril and a protein comprising the amino acid sequence shown by SEQ ID NO:24 or a protein homologous thereto or a variant thereof;
(a159) a combination of clinofibrate and a protein comprising the amino acid sequence shown by SEQ ID NO:23 or SEQ ID NO:34 or a protein homologous thereto or a variant thereof;
(a160) a combination of acetylcysteine and a protein comprising the amino acid sequence shown by SEQ ID NO:2 or SEQ ID NO:3 or a protein homologous thereto or a variant thereof;
(a161) a combination of buformin and a protein comprising the amino acid sequence shown by SEQ ID NO:57 or a protein homologous thereto or a variant thereof;
(a162) a combination of terguride and a protein comprising the amino acid sequence shown by SEQ ID NO:9 or a protein homologous thereto or a variant thereof;
(a163) a combination of stanozolol and a protein comprising the amino acid sequence shown by SEQ ID NO:16 or a protein homologous thereto or a variant thereof;
(a164) a combination of mestanolone and a protein comprising the amino acid sequence shown by SEQ ID NO:42 or a protein homologous thereto or a variant thereof;
(a165) a combination of pantethine and a protein comprising the amino acid sequence shown by SEQ ID NO:1 or a protein homologous thereto or a variant thereof;
(a166) a combination of limaprost and a protein comprising the amino acid sequence shown by SEQ ID NO:24 or a protein homologous thereto or a variant thereof;
(a167) a combination of sarpogrelate and a protein comprising the amino acid sequence shown by SEQ ID NO:27 or a protein homologous thereto or a variant thereof;
(a168) a combination of argatroban and a protein comprising the amino acid sequence shown by SEQ ID NO:23 or a protein homologous thereto or a variant thereof;
(a169) a combination of fludroxycortide and a protein comprising the amino acid sequence shown by SEQ ID NO:25 or a protein homologous thereto or a variant thereof;
(a170) a combination of sulfadoxine and a protein comprising the amino acid sequence shown by SEQ ID NO:23 or a protein homologous thereto or a variant thereof;
(a171) a combination of ubenimex and a protein comprising the amino acid sequence shown by SEQ ID NO:23 or a protein homologous thereto or a variant thereof;
(a172) a combination of celecoxib and a protein comprising the amino acid sequence shown by SEQ ID NO:23 or a protein homologous thereto or a variant thereof;
(a173) a combination of 6-furfurylaminopurine and a protein comprising the amino acid sequence shown by SEQ ID NO:57 or a protein homologous thereto or a variant thereof;
(a174) a combination of solasodine and a protein comprising the amino acid sequence shown by SEQ ID NO:24 or a protein homologous thereto or a variant thereof;
(a175) a combination of gossypol and a protein comprising the amino acid sequence shown by SEQ ID NO:23 or a protein homologous thereto or a variant thereof;
(a176) a combination of fluorocurarine and a protein comprising the amino acid sequence shown by SEQ ID NO:10 or a protein homologous thereto or a variant thereof;
(a177) a combination of pempidine and a protein comprising the amino acid sequence shown by SEQ ID NO:57 or a protein homologous thereto or a variant thereof;
(a178) a combination of nitrarine and a protein comprising the amino acid sequence shown by SEQ ID NO:46 or SEQ ID NO:57 or a protein homologous thereto or a variant thereof;
(a179) a combination of promazine and a protein comprising the amino acid sequence shown by SEQ ID NO:18 or a protein homologous thereto or a variant thereof;
(a180) a combination of sulfabenzamide and a protein comprising the amino acid sequence shown by SEQ ID NO:23 or a protein homologous thereto or a variant thereof;
(a181) a combination of althiazide and a protein comprising the amino acid sequence shown by SEQ ID NO:23 or a protein homologous thereto or a variant thereof;
(a182) a combination of α-ergocryptine and a protein comprising the amino acid sequence shown by SEQ ID NO:23 or SEQ ID NO:53 or a protein homologous thereto or a variant thereof;
(a183) a combination of ebselen and a protein comprising the amino acid sequence shown by SEQ ID NO:6 or a protein homologous thereto or a variant thereof;
(a184) a combination of furaltadone and a protein comprising the amino acid sequence shown by SEQ ID NO:10 or a protein homologous thereto or a variant thereof;
(a185) a combination of pyrithyldione and a protein comprising the amino acid sequence shown by SEQ ID NO:55 or a protein homologous thereto or a variant thereof;
(a186) a combination of benzthiazide and a protein comprising the amino acid sequence shown by SEQ ID NO:23 or SEQ ID NO:51 or a protein homologous thereto or a variant thereof;
(a187) a combination of levobunolol and a protein comprising the amino acid sequence shown by SEQ ID NO:44 or a protein homologous thereto or a variant thereof;
(a188) a combination of raloxifene and a protein comprising the amino acid sequence shown by SEQ ID NO:37 or a protein homologous thereto or a variant thereof;
(a189) a combination of luteolin and a protein comprising the amino acid sequence shown by SEQ ID NO:20 or SEQ ID NO:54 or a protein homologous thereto or a variant thereof;
(a190) a combination of valdecoxib and a protein comprising the amino acid sequence shown by SEQ ID NO:23 or a protein homologous thereto or a variant thereof;
(a191) a combination of carboprost and a protein comprising the amino acid sequence shown by SEQ ID NO:24 or SEQ ID NO:34 or a protein homologous thereto or a variant thereof;
(a192) a combination of gabexate and a protein comprising the amino acid sequence shown by SEQ ID NO:23 or a protein homologous thereto or a variant thereof.

2. The method according to claim 1, which comprises the following steps (a) to (c):
(a) a step for bringing the test substance into contact with the target protein Y;
(b) a step for measuring the functional level of the protein in the presence of the test substance, and comparing said functional level with the functional level of the protein in the absence of the test substance;
(c) a step for selecting a test substance that alters the functional level of the protein on the basis of the result of the comparison in (b) above.

3. The method according to claim 1, which comprises the following steps (a) to (c):
(a) a step for bringing the test substance into contact with cells allowing a measurement of the expression of the target protein Y or a gene that encodes the protein;
(b) a step for measuring the expression level of the gene in the cells in contact with the test substance, and comparing said expression level with the expression level of the gene in control cells not in contact with the test substance;
(c) a step for selecting a test substance that regulates the expression level of the gene on the basis of the result of the comparison in (b) above.

4. The method according to claim 1, which comprises the following steps (a) to (c):
(a) a step for bringing the test substance into contact with the target protein Y;
(b) a step for measuring the ability of the test substance to bind to the protein;
(c) a step for selecting a test substance capable of binding to the protein on the basis of the result from (b) above.

5. The method according to claim 1, which comprises the following steps (a) to (c):
(a) a step for bringing the test substance and a target protein Y-binding substance into contact with the target protein Y;
(b) a step for measuring the ability of the target protein Y-binding substance to bind to the protein in the presence of the test substance, and comparing said ability with an ability of the target protein Y-binding substance to bind to the protein in the absence of the test substance;
(c) a step for selecting a test substance that alters the ability of the target protein Y-binding substance to bind to the protein on the basis of the result of the comparison in (b) above.

6. A method for screening a substance capable of regulating a function associated with a target protein Y, which comprises comparing the ability of a test substance to bind to the target protein Y or the action associated with the test compound, with the ability of a bioactive substance X to bind to the target protein Y or the action associated with the bioactive substance, wherein the combination of the target protein Y and the bioactive substance X is any of the following (b1) to (b63):
(b1) a combination of a protein comprising the amino acid sequence shown by SEQ ID NO:1 or a protein homologous thereto or a variant thereof and ajmaline, celestine blue, conessine, difenidol, methoxy-6-harmalan, pimethixene, quinine, ritodrine, alimemazine, boldine, clofilium, paroxetine, trimethylcolchicic acid, antipyrine, cephaeline, ciclopirox, coniine (DL), doxazosin, sulfadimethoxine, pantethine or a derivative thereof capable of binding to the protein;
(b2) a combination of a protein comprising the amino acid sequence shown by SEQ ID NO:2 or a protein homologous thereto or a variant thereof and trimethylcolchicic acid, ajmaline, celestine blue, methoxy-6-harmalan, minaprine, ritodrine, scoulerine, alimemazine, acetylcysteine or a derivative thereof capable of binding to the protein;
(b3) a combination of a protein comprising the amino acid sequence shown by SEQ ID NO:3 or a protein homologous thereto or a variant thereof and celestine blue, ciclopirox, coniine (DL), tamoxifen, acetylcysteine, paracetamol or a derivative thereof capable of binding to the protein;
(b4) a combination of a protein comprising the amino acid sequence shown by SEQ ID NO:4 or a protein homologous thereto or a variant thereof and molsidomine or a derivative thereof capable of binding to the protein;
(b5) a combination of a protein comprising the amino acid sequence shown by SEQ ID NO:5 or a protein homologous thereto or a variant thereof and trimetazidine or a derivative thereof capable of binding to the protein;
(b6) a combination of a protein comprising the amino acid sequence shown by SEQ ID NO:6 or a protein homologous thereto or a variant thereof and α-lobeline (−), ebselen or a derivative thereof capable of binding to the protein;
(b7) a combination of a protein comprising the amino acid sequence shown by SEQ ID NO:7 or a protein homologous thereto or a variant thereof and moroxydine or a derivative thereof capable of binding to the protein;
(b8) a combination of a protein comprising the amino acid sequence shown by SEQ ID NO:8 or a protein homologous thereto or a variant thereof and xylazine or a derivative thereof capable of binding to the protein;
(b9) a combination of a protein comprising the amino acid sequence shown by SEQ ID NO:9 or a protein homologous thereto or a variant thereof and terguride, iobenguane or a derivative thereof capable of binding to the protein;
(b10) a combination of a protein comprising the amino acid sequence shown by SEQ ID NO:10 or a protein homologous thereto or a variant thereof and quinine, eburnamonine, fluorocurarine, furaltadone or a derivative thereof capable of binding to the protein;
(b11) a combination of a protein comprising the amino acid sequence shown by SEQ ID NO:11 or a protein homologous thereto or a variant thereof and hydroflumethiazide or a derivative thereof capable of binding to the protein;
(b12) a combination of a protein comprising the amino acid sequence shown by SEQ ID NO:12 or a protein homologous thereto or a variant thereof and methimazole or a derivative thereof capable of binding to the protein;
(b13) a combination of a protein comprising the amino acid sequence shown by SEQ ID NO:13 or a protein homologous thereto or a variant thereof and risperidone or a derivative thereof capable of binding to the protein;
(b14) a combination of a protein comprising the amino acid sequence shown by SEQ ID NO:14 or a protein homologous thereto or a variant thereof and bupivacaine or a derivative thereof capable of binding to the protein;
(b15) a combination of a protein comprising the amino acid sequence shown by SEQ ID NO:15 or a protein homologous thereto or a variant thereof and loperamide, clofazimine, dipyridamole or a derivative thereof capable of binding to the protein;
(b16) a combination of a protein comprising the amino acid sequence shown by SEQ ID NO:16 or a protein homologous thereto or a variant thereof and stanozolol, methyclothiazide or a derivative thereof capable of binding to the protein;
(b17) a combination of a protein comprising the amino acid sequence shown by SEQ ID NO:17 or a protein homologous thereto or a variant thereof and chromomycin A3, meclofenamic acid, saquinavir or a derivative thereof capable of binding to the protein;
(b18) a combination of a protein comprising the amino acid sequence shown by SEQ ID NO:18 or a protein homologous thereto or a variant thereof and promazine, pranlukast or a derivative thereof capable of binding to the protein;
(b19) a combination of a protein comprising the amino acid sequence shown by SEQ ID NO:19 or a protein homologous thereto or a variant thereof and dihydrostreptomycin, iproniazid, nefopam or a derivative thereof capable of binding to the protein;
(b20) a combination of a protein comprising the amino acid sequence shown by SEQ ID NO:20 or a protein homologous thereto or a variant thereof and quercetin, luteolin, pranlukast or a derivative thereof capable of binding to the protein;
(b21) a combination of a protein comprising the amino acid sequence shown by SEQ ID NO:21 or a protein homologous thereto or a variant thereof and pranlukast or a derivative thereof capable of binding to the protein;
(b22) a combination of a protein comprising the amino acid sequence shown by SEQ ID NO:22 or a protein homologous thereto or a variant thereof and clemizole, fenbendazole, harmol, ifosfamide, piperlongumine, propranolol or a derivative thereof capable of binding to the protein;
(b23) a combination of a protein comprising the amino acid sequence shown by SEQ ID NO:23 or a protein homologous thereto or a variant thereof and acetohexamide, benzethonium, clomiphene, cyclobenzaprine, flupentixol, guanfacine, maprotiline, perhexiline, probenecid, clinofibrate, celecoxib, gossypol, althiazide, α-ergocryptine, gabexate, clenbuterol, etodolac, misoprostol, ubenimex, clopamide, glibenclamide, glipizide, isoxicam, sulfaphenazole, thioproperazine, thiothixene(cis), tolbutamide, methyclothiazide, argatroban, sulfadoxine, sulfabenzamide, benzthiazide, valdecoxib or a derivative thereof capable of binding to the protein;
(b24) a combination of a protein comprising the amino acid sequence shown by SEQ ID NO:24 or a protein homologous thereto or a variant thereof and acetohexamide, isradipine, mupirocin, limaprost, solasodine, alacepril, carboprost or a derivative thereof capable of binding to the protein;
(b25) a combination of a protein comprising the amino acid sequence shown by SEQ ID NO:25 or a protein homologous thereto or a variant thereof and metergotamine, methoxamine, paroxetine, dizocilpine, fluvoxamine, 3-hydroxykynurenine, nimetazepam, fludroxycortide or a derivative thereof capable of binding to the protein;
(b26) a combination of a protein comprising the amino acid sequence shown by SEQ ID NO:26 or a protein homologous thereto or a variant thereof and fenoprofen or a derivative thereof capable of binding to the protein;
(b27) a combination of a protein comprising the amino acid sequence shown by SEQ ID NO:27 or a protein homologous thereto or a variant thereof and acenocoumarol, budesonide, chlorogenic acid, chlorothiazide, diclofenac, diperodon, DO897/99, nimesulide, thioproperazine, sarpogrelate or a derivative thereof capable of binding to the protein;
(b28) a combination of a protein comprising the amino acid sequence shown by SEQ ID NO:28 or a protein homologous thereto or a variant thereof and acetylsalicylsalicylic acid or a derivative thereof capable of binding to the protein;
(b29) a combination of a protein comprising the amino acid sequence shown by SEQ ID NO:29 or a protein homologous thereto or a variant thereof and buspirone or a derivative thereof capable of binding to the protein;
(b30) a combination of a protein comprising the amino acid sequence shown by SEQ ID NO:30 or a protein homologous thereto or a variant thereof and dopamine, α-methyl-5-hydroxytryptamine or a derivative thereof capable of binding to the protein;
(b31) a combination of a protein comprising the amino acid sequence shown by SEQ ID NO:31 or a protein homologous thereto or a variant thereof and cisapride or a derivative thereof capable of binding to the protein;
(b32) a combination of a protein comprising the amino acid sequence shown by SEQ ID NO:32 or a protein homologous thereto or a variant thereof and berberine, celestine blue, diflunisal, mebendazole, tranilast or a derivative thereof capable of binding to the protein;
(b33) a combination of a protein comprising the amino acid sequence shown by SEQ ID NO:33 or a protein homologous thereto or a variant thereof and bromperidol, coralyne or a derivative thereof capable of binding to the protein;
(b34) a combination of a protein comprising the amino acid sequence shown by SEQ ID NO:34 or a protein homologous thereto or a variant thereof and DO897/99, domperidone, flupentixol, fluphenazine, L-thyroxine, trifluoperazine, clinofibrate, acetohexamide, chromomycin A3, carboprost or a derivative thereof capable of binding to the protein;
(b35) a combination of a protein comprising the amino acid sequence shown by SEQ ID NO:35 or a protein homologous thereto or a variant thereof and alfuzosin, clobetasone, doxazosin, pranlukast, risperidone or a derivative thereof capable of binding to the protein;
(b36) a combination of a protein comprising the amino acid sequence shown by SEQ ID NO:36 or a protein homologous thereto or a variant thereof and acetopromazine, cyclopentolate, perhexiline, phenformin, pyrilamine, terconazole, tobramycin, amoxapine, cephaeline, clenbuterol, domperidone, minocycline, moxalactam or a derivative thereof capable of binding to the protein;
(b37) a combination of a protein comprising the amino acid sequence shown by SEQ ID NO:37 or a protein homologous thereto or a variant thereof and glibenclamide, raloxifene, clofazimine or a derivative thereof capable of binding to the protein;
(b38) a combination of a protein comprising the amino acid sequence shown by SEQ ID NO:38 or a protein homologous thereto or a variant thereof and albendazole or a derivative thereof capable of binding to the protein;
(b39) a combination of a protein comprising the amino acid sequence shown by SEQ ID NO:39 or a protein homologous thereto or a variant thereof and bezafibrate or a derivative thereof capable of binding to the protein;
(b40) a combination of a protein comprising the amino acid sequence shown by SEQ ID NO:40 or a protein homologous thereto or a variant thereof and pirenzepine or a derivative thereof capable of binding to the protein;
(b41) a combination of a protein comprising the amino acid sequence shown by SEQ ID NO:41 or a protein homologous thereto or a variant thereof and rescinnamine or a derivative thereof capable of binding to the protein;
(b42) a combination of a protein comprising the amino acid sequence shown by SEQ ID NO:42 or a protein homologous thereto or a variant thereof and benzbromarone, pranlukast, mifepristone, mestanolone or a derivative thereof capable of binding to the protein;
(b43) a combination of a protein comprising the amino acid sequence shown by SEQ ID NO:43 or a protein homologous thereto or a variant thereof and hydroxytacrine(R,S), metergotamine, metaproterenol or a derivative thereof capable of binding to the protein;
(b44) a combination of a protein comprising the amino acid sequence shown by SEQ ID NO:44 or a protein homologous thereto or a variant thereof and eburnamonine, levobunolol or a derivative thereof capable of binding to the protein;
(b45) a combination of a protein comprising the amino acid sequence shown by SEQ ID NO:45 or a protein homologous thereto or a variant thereof and norharman, pyrilamine or a derivative thereof capable of binding to the protein;
(b46) a combination of a protein comprising the amino acid sequence shown by SEQ ID NO:46 or a protein homologous thereto or a variant thereof and celestine blue, nitrarine or a derivative thereof capable of binding to the protein;
(b47) a combination of a protein comprising the amino acid sequence shown by SEQ ID NO:47 or a protein homologous thereto or a variant thereof and clemizole or a derivative thereof capable of binding to the protein;
(b48) a combination of a protein comprising the amino acid sequence shown by SEQ ID NO:48 or a protein homologous thereto or a variant thereof and clobazam or a derivative thereof capable of binding to the protein;
(b49) a combination of a protein comprising the amino acid sequence shown by SEQ ID NO:49 or a protein homologous thereto or a variant thereof and josamycin, oxytocin, clarithromycin or a derivative thereof capable of binding to the protein;
(b50) a combination of a protein comprising the amino acid sequence shown by SEQ ID NO:50 or a protein homologous thereto or a variant thereof and leuprolide, cyclosporine A or a derivative thereof capable of binding to the protein;
(b51) a combination of a protein comprising the amino acid sequence shown by SEQ ID NO:51 or a protein homologous thereto or a variant thereof and dichlorphenamide, benzthiazide or a derivative thereof capable of binding to the protein;
(b52) a combination of a protein comprising the amino acid sequence shown by SEQ ID NO:52 or a protein homologous thereto or a variant thereof and hydroxychloroquine, furazolidone, metanephrine (D,L) or a derivative thereof capable of binding to the protein;
(b53) a combination of a protein comprising the amino acid sequence shown by SEQ ID NO:53 or a protein homologous thereto or a variant thereof and benzbromarone, benzethonium, clofazimine, domperidone, doxazosin, gramicidin, α-ergocryptine, bicartamide, rescinnamine, saquinavir, syrosingopine, pranlukast or a derivative thereof capable of binding to the protein;
(b54) a combination of a protein comprising the amino acid sequence shown by SEQ ID NO:54 or a protein homologous thereto or a variant thereof and benzbromarone, clofazimine, domperidone, nicardipine, quercetin, ebastine, actinomycin D, loperamide, pranlukast, luteolin or a derivative thereof capable of binding to the protein;
(b55) a combination of a protein comprising the amino acid sequence shown by SEQ ID NO:55 or a protein homologous thereto or a variant thereof and pyrithyldione or a derivative thereof capable of binding to the protein;
(b56) a combination of a protein comprising the amino acid sequence shown by SEQ ID NO:56 or a protein homologous thereto or a variant thereof and chlordiazepoxide, flumequine or a derivative thereof capable of binding to the protein;
(b57) a combination of a protein comprising the amino acid sequence shown by SEQ ID NO:57 or a protein homologous thereto or a variant thereof and buformin, 6-furfurylaminopurine, nitrarine, pempidine or a derivative thereof capable of binding to the protein;
(b58) a combination of a protein comprising the amino acid sequence shown by SEQ ID NO:58 or a protein homologous thereto or a variant thereof and viloxazine or a derivative thereof capable of binding to the protein;
(b59) a combination of a protein comprising the amino acid sequence shown by SEQ ID NO:59 or a protein homologous thereto or a variant thereof and cefazolin, fenbufen, ketoprofen, colchicine, doxycycline, gabapentin, lidoflazine, probenecid or a derivative thereof capable of binding to the protein;
(b60) a combination of a protein comprising the amino acid sequence shown by SEQ ID NO:60 or a protein homologous thereto or a variant thereof and benzydamine, clenbuterol or a derivative thereof capable of binding to the protein;
(b61) a combination of a protein comprising the amino acid sequence shown by SEQ ID NO:61 or a protein homologous thereto or a variant thereof and benzethonium, fluphenazine, GBR12909, doxazosin, procaine, quinacrine or a derivative thereof capable of binding to the protein;
(b62) a combination of a protein comprising the amino acid sequence shown by SEQ ID NO:62 or a protein homologous thereto or a variant thereof and azithromycin, colistin or a derivative thereof capable of binding to the protein;
(b63) a combination of a protein comprising the amino acid sequence shown by SEQ ID NO:63 or a protein homologous thereto or a variant thereof and protriptyline, maprotiline or a derivative thereof capable of binding to the protein.

7. (canceled)

8. (canceled)

9. An agent of regulating an action associated with a bioactive substance X, which comprises a substance that regulates the expression or function of a target protein Y or a gene that encodes the protein, wherein the combination of the bioactive substance X and the target protein Y is any of (a1) to (a192) of claim 1.

10. The agent according to claim 9, wherein the substance that regulates the expression or function of a target protein Y or a gene that encodes the protein is a substance that suppresses the expression or function of the gene.

11. The agent according to claim 10, wherein the substance that suppresses the expression or function of a target protein Y or a gene that encodes the protein is antisense nucleic acid, ribozyme, decoy nucleic acid, siRNA, antibody or dominant negative mutant, or an expression vector thereof.

12. The agent according to claim 9, which comprises the target protein Y, or an expression vector comprising a nucleic acid that encodes the protein.

13. An agent of regulating a function associated with a target protein Y, which comprises a bioactive substance X, wherein the combination of the bioactive substance X and the target protein Y is any of (b1) to (b63) of claim 6.

14. A method of producing a derivative of bioactive substance X, which comprises derivatizing the bioactive substance X so as to be able to regulate the expression or function of a target protein Y or a gene that encodes the protein, wherein the combination if the bioactive substance X and the target protein Y is any of (a1) to (a192) of claim 1.

15. A method of producing a derivative of a substance capable of regulating a function associated with a target protein Y, which comprises derivatizing a bioactive substance X so as to be able to regulate the ability of the bioactive substance X to bind to the target protein Y, wherein the combination of the bioactive substance X and the target protein Y is any of (b1) to (b63) of claim 6.

16. (canceled)

17. (canceled)

18. A complex comprising a bioactive substance X and a target protein Y thereof, wherein the combination of the bioactive substance X and the target protein Y is any of the following (a1) to (a192) or (b1) to (b63):
(a1) a combination of trimethylcolchicic acid and a protein comprising the amino acid sequence shown by SEQ ID NO:1 or SEQ ID NO:2 or a protein homologous thereto or a variant thereof;
(a2) a combination of acenocoumarol and a protein comprising the amino acid sequence shown by SEQ ID NO:27 or a protein homologous thereto or a variant thereof;
(a3) a combination of paracetamol and a protein comprising the amino acid sequence shown by SEQ ID NO:3 or a protein homologous thereto or a variant thereof;
(a4) a combination of acetohexamide and a protein comprising the amino acid sequence shown by SEQ ID NO:23, SEQ ID NO:24 or SEQ ID NO:34 or a protein homologous thereto or a variant thereof;
(a5) a combination of acetopromazine and a protein comprising the amino acid sequence shown by SEQ ID NO:36 or a protein homologous thereto or a variant thereof;
(a6) a combination of actinomycin D and a protein comprising the amino acid sequence shown by SEQ ID NO:54 or a protein homologous thereto or a variant thereof;
(a7) a combination of ajmaline and a protein comprising the amino acid sequence shown by SEQ ID NO:1 or SEQ ID NO:2 or a protein homologous thereto or a variant thereof;
(a8) a combination of albendazole and a protein comprising the amino acid sequence shown by SEQ ID NO:38 or a protein homologous thereto or a variant thereof;
(a9) a combination of alfuzosin and a protein comprising the amino acid sequence shown by SEQ ID NO:35 or a protein homologous thereto or a variant thereof;
(a10) a combination of α-methyl-5-hydroxytryptamine and a protein comprising the amino acid sequence shown by SEQ ID NO:30 or a protein homologous thereto or a variant thereof;
(a11) a combination of amoxapine and a protein comprising the amino acid sequence shown by SEQ ID NO:36 or a protein homologous thereto or a variant thereof;
(a12) a combination of antipyrine and a protein comprising the amino acid sequence shown by SEQ ID NO:1 or a protein homologous thereto or a variant thereof;
(a13) a combination of azithromycin and a protein comprising the amino acid sequence shown by SEQ ID NO:62 or a protein homologous thereto or a variant thereof;
(a14) a combination of benzbromarone and a protein comprising the amino acid sequence shown by SEQ ID NO:42, SEQ ID NO:53 or SEQ ID NO:54 or a protein homologous thereto or a variant thereof;
(a15) a combination of benzethonium and a protein comprising the amino acid sequence shown by SEQ ID NO:23, SEQ ID NO:53 or SEQ ID NO:61 or a protein homologous thereto or a variant thereof;
(a16) a combination of benzydamine and a protein comprising the amino acid sequence shown by SEQ ID NO:60 or a protein homologous thereto or a variant thereof;
(a17) a combination of berberine and a protein comprising the amino acid sequence shown by SEQ ID NO:32 or a protein homologous thereto or a variant thereof;
(a18) a combination of bezafibrate and a protein comprising the amino acid sequence shown by SEQ ID NO:39 or a protein homologous thereto or a variant thereof;
(a19) a combination of bicartamide and a protein comprising the amino acid sequence shown by SEQ ID NO:53 or a protein homologous thereto or a variant thereof;
(a20) a combination of boldine and a protein comprising the amino acid sequence shown by SEQ ID NO:1 or a protein homologous thereto or a variant thereof;
(a21) a combination of bromperidol and a protein comprising the amino acid sequence shown by SEQ ID NO:33 or a protein homologous thereto or a variant thereof;
(a22) a combination of budesonide and a protein comprising the amino acid sequence shown by SEQ ID NO:27 or a protein homologous thereto or a variant thereof;
(a23) a combination of bupivacaine and a protein comprising the amino acid sequence shown by SEQ ID NO:14 or a protein homologous thereto or a variant thereof;
(a24) a combination of buspirone and a protein comprising the amino acid sequence shown by SEQ ID NO:29 or a protein homologous thereto or a variant thereof;
(a25) a combination of cefazolin and a protein comprising the amino acid sequence shown by SEQ ID NO:59 or a protein homologous thereto or a variant thereof;
(a26) a combination of celestine blue and a protein comprising the amino acid sequence shown by SEQ ID NO:1, SEQ ID NO:2, SEQ ID NO:3, SEQ ID NO:32 or SEQ ID NO:46 or a protein homologous thereto or a variant thereof;
(a27) a combination of cephaeline and a protein comprising the amino acid sequence shown by SEQ ID NO:1 or SEQ ID NO:36 or a protein homologous thereto or a variant thereof;
(a28) a combination of chlordiazepoxide and a protein comprising the amino acid sequence shown by SEQ ID NO:56 or a protein homologous thereto or a variant thereof;
(a29) a combination of chlorogenic acid and a protein comprising the amino acid sequence shown by SEQ ID NO:27 or a protein homologous thereto or a variant thereof;
(a30) a combination of chlorothiazide and a protein comprising the amino acid sequence shown by SEQ ID NO:27 or a protein homologous thereto or a variant thereof;
(a31) a combination of chromomycin A3 and a protein comprising the amino acid sequence shown by SEQ ID NO:17 or SEQ ID NO:34 or a protein homologous thereto or a variant thereof;
(a32) a combination of ciclopirox and a protein comprising the amino acid sequence shown by SEQ ID NO:1 or SEQ ID NO:3 or a protein homologous thereto or a variant thereof;
(a33) a combination of cisapride and a protein comprising the amino acid sequence shown by SEQ ID NO:31 or a protein homologous thereto or a variant thereof;
(a34) a combination of clarithromycin and a protein comprising the amino acid sequence shown by SEQ ID NO:49 or a protein homologous thereto or a variant thereof;
(a35) a combination of clemizole and a protein comprising the amino acid sequence shown by SEQ ID NO:22 or SEQ ID NO:47 or a protein homologous thereto or a variant thereof;
(a36) a combination of clenbuterol and a protein comprising the amino acid sequence shown by SEQ ID NO:23, SEQ ID NO:36 or SEQ ID NO:60 or a protein homologous thereto or a variant thereof;
(a37) a combination of clobetasone and a protein comprising the amino acid sequence shown by SEQ ID NO:35 or a protein homologous thereto or a variant thereof;
(a38) a combination of clofazimine and a protein comprising the amino acid sequence shown by SEQ ID NO:15, SEQ ID NO:37, SEQ ID NO:53 or SEQ ID NO:54 or a protein homologous thereto or a variant thereof;
(a39) a combination of clofilium and a protein comprising the amino acid sequence shown by SEQ ID NO:1 or a protein homologous thereto or a variant thereof;
(a40) a combination of clomiphene and a protein comprising the amino acid sequence shown by SEQ ID NO:23 or a protein homologous thereto or a variant thereof;
(a41) a combination of clopamide and a protein comprising the amino acid sequence shown by SEQ ID NO:23 or a protein homologous thereto or a variant thereof;
(a42) a combination of colchicine and a protein comprising the amino acid sequence shown by SEQ ID NO:59 or a protein homologous thereto or a variant thereof;
(a43) a combination of colistin and a protein comprising the amino acid sequence shown by SEQ ID NO:62 or a protein homologous thereto or a variant thereof;
(a44) a combination of conessine and a protein comprising the amino acid sequence shown by SEQ ID NO:1 or a protein homologous thereto or a variant thereof;
(a45) a combination of coniine (DL) and a protein comprising the amino acid sequence shown by SEQ ID NO:1 or SEQ ID NO:3 or a protein homologous thereto or a variant thereof;
(a46) a combination of coralyne and a protein comprising the amino acid sequence shown by SEQ ID NO:33 or a protein homologous thereto or a variant thereof;
(a47) a combination of cyclobenzaprine and a protein comprising the amino acid sequence shown by SEQ ID NO:23 or a protein homologous thereto or a variant thereof;
(a48) a combination of cyclopentolate and a protein comprising the amino acid sequence shown by SEQ ID NO:36 or a protein homologous thereto or a variant thereof;
(a49) a combination of cyclosporine A and a protein comprising the amino acid sequence shown by SEQ ID NO:50 or a protein homologous thereto or a variant thereof;
(a50) a combination of diclofenac and a protein comprising the amino acid sequence shown by SEQ ID NO:27 or a protein homologous thereto or a variant thereof;
(a51) a combination of dichlorphenamide and a protein comprising the amino acid sequence shown by SEQ ID NO:51 or a protein homologous thereto or a variant thereof;
(a52) a combination of diflunisal and a protein comprising the amino acid sequence shown by SEQ ID NO:32 or a protein homologous thereto or a variant thereof;
(a53) a combination of dihydrostreptomycin and a protein comprising the amino acid sequence shown by SEQ ID NO:19 or a protein homologous thereto or a variant thereof;
(a54) a combination of diperodon and a protein comprising the amino acid sequence shown by SEQ ID NO:27 or a protein homologous thereto or a variant thereof;
(a55) a combination of difenidol and a protein comprising the amino acid sequence shown by SEQ ID NO:1 or a protein homologous thereto or a variant thereof;
(a56) a combination of dipyridamole and a protein comprising the amino acid sequence shown by SEQ ID NO:15 or a protein homologous thereto or a variant thereof;
(a57) a combination of dizocilpine and a protein comprising the amino acid sequence shown by SEQ ID NO:25 or a protein homologous thereto or a variant thereof;
(a58) a combination of DO897/99 and a protein comprising the amino acid sequence shown by SEQ ID NO:27 or SEQ ID NO:34 or a protein homologous thereto or a variant thereof;
(a59) a combination of domperidone and a protein comprising the amino acid sequence shown by SEQ ID NO:34, SEQ ID NO:36, SEQ ID NO:53 or SEQ ID NO:54 or a protein homologous thereto or a variant thereof;
(a60) a combination of dopamine and a protein comprising the amino acid sequence shown by SEQ ID NO:30 or a protein homologous thereto or a variant thereof;
(a61) a combination of doxazosin and a protein comprising the amino acid sequence shown by SEQ ID NO:1, SEQ ID NO:35, SEQ ID NO:53 or SEQ ID NO:61 or a protein homologous thereto or a variant thereof;
(a62) a combination of doxycycline and a protein comprising the amino acid sequence shown by SEQ ID NO:59 or a protein homologous thereto or a variant thereof;
(a63) a combination of eburnamonine and a protein comprising the amino acid sequence shown by SEQ ID NO:10 or SEQ ID NO:44 or a protein homologous thereto or a variant thereof;
(a64) a combination of etodolac and a protein comprising the amino acid sequence shown by SEQ ID NO:23 or a protein homologous thereto or a variant thereof;
(a65) a combination of fenbendazole and a protein comprising the amino acid sequence shown by SEQ ID NO:22 or a protein homologous thereto or a variant thereof;
(a66) a combination of fenbufen and a protein comprising the amino acid sequence shown by SEQ ID NO:59 or a protein homologous thereto or a variant thereof;
(a67) a combination of fenoprofen and a protein comprising the amino acid sequence shown by SEQ ID NO:26 or a protein homologous thereto or a variant thereof;
(a68) a combination of flumequine and a protein comprising the amino acid sequence shown by SEQ ID NO:56 or a protein homologous thereto or a variant thereof;
(a69) a combination of flupentixol and a protein comprising the amino acid sequence shown by SEQ ID NO:23 or SEQ ID NO:34 or a protein homologous thereto or a variant thereof;
(a70) a combination of fluphenazine and a protein comprising the amino acid sequence shown by SEQ ID NO:34 or SEQ ID NO:61 or a protein homologous thereto or a variant thereof;
(a71) a combination of fluvoxamine and a protein comprising the amino acid sequence shown by SEQ ID NO:25 or a protein homologous thereto or a variant thereof;
(a72) a combination of furazolidone and a protein comprising the amino acid sequence shown by SEQ ID NO:52 or a protein homologous thereto or a variant thereof;
(a73) a combination of gabapentin and a protein comprising the amino acid sequence shown by SEQ ID NO:59 or a protein homologous thereto or a variant thereof;
(a74) a combination of GBR12909 and a protein comprising the amino acid sequence shown by SEQ ID NO:61 or a protein homologous thereto or a variant thereof;
(a75) a combination of glibenclamide and a protein comprising the amino acid sequence shown by SEQ ID NO:23 or SEQ ID NO:37 or a protein homologous thereto or a variant thereof;
(a76) a combination of glipizide and a protein comprising the amino acid sequence shown by SEQ ID NO:23 or a protein homologous thereto or a variant thereof;
(a77) a combination of gramicidin and a protein comprising the amino acid sequence shown by SEQ ID NO:53 or a protein homologous thereto or a variant thereof;
(a78) a combination of guanfacine and a protein comprising the amino acid sequence shown by SEQ ID NO:23 or a protein homologous thereto or a variant thereof;
(a79) a combination of harmol and a protein comprising the amino acid sequence shown by SEQ ID NO:22 or a protein homologous thereto or a variant thereof;
(a80) a combination of hydroflumethiazide and a protein comprising the amino acid sequence shown by SEQ ID NO:11 or a protein homologous thereto or a variant thereof;
(a81) a combination of hydroxychloroquine and a protein comprising the amino acid sequence shown by SEQ ID NO:52 or a protein homologous thereto or a variant thereof;
(a82) a combination of hydroxytacrine(R,S) and a protein comprising the amino acid sequence shown by SEQ ID NO:43 or a protein homologous thereto or a variant thereof;
(a83) a combination of ifosfamide and a protein comprising the amino acid sequence shown by SEQ ID NO:22 or a protein homologous thereto or a variant thereof;
(a84) a combination of iobenguane and a protein comprising the amino acid sequence shown by SEQ ID NO:9 or a protein homologous thereto or a variant thereof;
(a85) a combination of iproniazid and a protein comprising the amino acid sequence shown by SEQ ID NO:19 or a protein homologous thereto or a variant thereof;
(a86) a combination of isoxicam and a protein comprising the amino acid sequence shown by SEQ ID NO:23 or a protein homologous thereto or a variant thereof;
(a87) a combination of isradipine and a protein comprising the amino acid sequence shown by SEQ ID NO:24 or a protein homologous thereto or a variant thereof;
(a88) a combination of josamycin and a protein comprising the amino acid sequence shown by SEQ ID NO:49 or a protein homologous thereto or a variant thereof;
(a89) a combination of ketoprofen and a protein comprising the amino acid sequence shown by SEQ ID NO:59 or a protein homologous thereto or a variant thereof;
(a90) a combination of 3-hydroxykynurenine and a protein comprising the amino acid sequence shown by SEQ ID NO:25 or a protein homologous thereto or a variant thereof;
(a91) a combination of leuprolide and a protein comprising the amino acid sequence shown by SEQ ID NO:50 or a protein homologous thereto or a variant thereof;
(a92) a combination of L-thyroxine and a protein comprising the amino acid sequence shown by SEQ ID NO:34 or a protein homologous thereto or a variant thereof;
(a93) a combination of lidoflazine and a protein comprising the amino acid sequence shown by SEQ ID NO:59 or a protein homologous thereto or a variant thereof;
(a94) a combination of α-lobeline (−) and a protein comprising the amino acid sequence shown by SEQ ID NO:6 or a protein homologous thereto or a variant thereof;
(a95) a combination of loperamide and a protein comprising the amino acid sequence shown by SEQ ID NO:15 or SEQ ID NO:54 or a protein homologous thereto or a variant thereof;
(a96) a combination of maprotiline and a protein comprising the amino acid sequence shown by SEQ ID NO:23 or SEQ ID NO:63 or a protein homologous thereto or a variant thereof;
(a97) a combination of mebendazole and a protein comprising the amino acid sequence shown by SEQ ID NO:32 or a protein homologous thereto or a variant thereof;
(a98) a combination of meclofenamic acid and a protein comprising the amino acid sequence shown by SEQ ID NO:17 or a protein homologous thereto or a variant thereof;
(a99) a combination of metanephrine (D,L) a protein comprising the amino acid sequence shown by SEQ ID NO:52 or a protein homologous thereto or a variant thereof;
(a100) a combination of metaproterenol and a protein comprising the amino acid sequence shown by SEQ ID NO:43 or a protein homologous thereto or a variant thereof;
(a101) a combination of metergotamine and a protein comprising the amino acid sequence shown by SEQ ID NO:25 or SEQ ID NO:43 or a protein homologous thereto or a variant thereof;
(a102) a combination of methimazole and a protein comprising the amino acid sequence shown by SEQ ID NO:12 or a protein homologous thereto or a variant thereof;
(a103) a combination of methoxamine and a protein comprising the amino acid sequence shown by SEQ ID NO:25 or a protein homologous thereto or a variant thereof;
(a104) a combination of methoxy-6-harmalan and a protein comprising the amino acid sequence shown by SEQ ID NO:1 or SEQ ID NO:2 or a protein homologous thereto or a variant thereof;
(a105) a combination of mifepristone and a protein comprising the amino acid sequence shown by SEQ ID NO:42 or a protein homologous thereto or a variant thereof;
(a106) a combination of minaprine and a protein comprising the amino acid sequence shown by SEQ ID NO:2 or a protein homologous thereto or a variant thereof;
(a107) a combination of minocycline and a protein comprising the amino acid sequence shown by SEQ ID NO:36 or a protein homologous thereto or a variant thereof;
(a108) a combination of misoprostol and a protein comprising the amino acid sequence shown by SEQ ID NO:23 or a protein homologous thereto or a variant thereof;
(a109) a combination of molsidomine and a protein comprising the amino acid sequence shown by SEQ ID NO:4 or a protein homologous thereto or a variant thereof;
(a110) a combination of moroxydine and a protein comprising the amino acid sequence shown by SEQ ID NO:7 or a protein homologous thereto or a variant thereof;
(a111) a combination of moxalactam and a protein comprising the amino acid sequence shown by SEQ ID NO:36 or a protein homologous thereto or a variant thereof;
(a112) a combination of mupirocin and a protein comprising the amino acid sequence shown by SEQ ID NO:24 or a protein homologous thereto or a variant thereof;
(a113) a combination of nefopam and a protein comprising the amino acid sequence shown by SEQ ID NO:19 or a protein homologous thereto or a variant thereof;
(a114) a combination of nicardipine and a protein comprising the amino acid sequence shown by SEQ ID NO:54 or a protein homologous thereto or a variant thereof;
(a115) a combination of nimesulide and a protein comprising the amino acid sequence shown by SEQ ID NO:27 or a protein homologous thereto or a variant thereof;
(a116) a combination of norharman and a protein comprising the amino acid sequence shown by SEQ ID NO:45 or a protein homologous thereto or a variant thereof;
(a117) a combination of oxytocin and a protein comprising the amino acid sequence shown by SEQ ID NO:49 or a protein homologous thereto or a variant thereof;
(a118) a combination of paroxetine and a protein comprising the amino acid sequence shown by SEQ ID NO:1 or SEQ ID NO:25 or a protein homologous thereto or a variant thereof;
(a119) a combination of perhexiline and a protein comprising the amino acid sequence shown by SEQ ID NO:23 or SEQ ID NO:36 or a protein homologous thereto or a variant thereof;
(a120) a combination of phenformin and a protein comprising the amino acid sequence shown by SEQ ID NO:36 or a protein homologous thereto or a variant thereof;
(a121) a combination of pimethixene and a protein comprising the amino acid sequence shown by SEQ ID NO:1 or a protein homologous thereto or a variant thereof;
(a122) a combination of piperlongumine and a protein comprising the amino acid sequence shown by SEQ ID NO:22 or a protein homologous thereto or a variant thereof;
(a123) a combination of pirenzepine and a protein comprising the amino acid sequence shown by SEQ ID NO:40 or a protein homologous thereto or a variant thereof;
(a124) a combination of probenecid and a protein comprising the amino acid sequence shown by SEQ ID NO:23 or SEQ ID NO:59 or a protein homologous thereto or a variant thereof;
(a125) a combination of procaine and a protein comprising the amino acid sequence shown by SEQ ID NO:61 or a protein homologous thereto or a variant thereof;
(a126) a combination of propranolol and a protein comprising the amino acid sequence shown by SEQ ID NO:22 or a protein homologous thereto or a variant thereof;
(a127) a combination of protriptyline and a protein comprising the amino acid sequence shown by SEQ ID NO:63 or a protein homologous thereto or a variant thereof;
(a128) a combination of pyrilamine and a protein comprising the amino acid sequence shown by SEQ ID NO:36 or SEQ ID NO:45 or a protein homologous thereto or a variant thereof;
(a129) a combination of quercetin and a protein comprising the amino acid sequence shown by SEQ ID NO:20 or SEQ ID NO:54 or a protein homologous thereto or a variant thereof;
(a130) a combination of quinacrine and a protein comprising the amino acid sequence shown by SEQ ID NO:61 or a protein homologous thereto or a variant thereof;
(a131) a combination of quinine and a protein comprising the amino acid sequence shown by SEQ ID NO:1 or SEQ ID NO:10 or a protein homologous thereto or a variant thereof;
(a132) a combination of rescinnamine and a protein comprising the amino acid sequence shown by SEQ ID NO:41 or SEQ ID NO:53 or a protein homologous thereto or a variant thereof;
(a133) a combination of risperidone and a protein comprising the amino acid sequence shown by SEQ ID NO:13 or SEQ ID NO:35 or a protein homologous thereto or a variant thereof;
(a134) a combination of ritodrine and a protein comprising the amino acid sequence shown by SEQ ID NO:1 or SEQ ID NO:2 or a protein homologous thereto or a variant thereof;
(a135) a combination of saquinavir and a protein comprising the amino acid sequence shown by SEQ ID NO:17 or SEQ ID NO:53 or a protein homologous thereto or a variant thereof;
(a136) a combination of scoulerine and a protein comprising the amino acid sequence shown by SEQ ID NO:2 or a protein homologous thereto or a variant thereof;
(a137) a combination of sulfadimethoxine and a protein comprising the amino acid sequence shown by SEQ ID NO:1 or a protein homologous thereto or a variant thereof;
(a138) a combination of sulfaphenazole and a protein comprising the amino acid sequence shown by SEQ ID NO:23 or a protein homologous thereto or a variant thereof;
(a139) a combination of syrosingopine and a protein comprising the amino acid sequence shown by SEQ ID NO:53 or a protein homologous thereto or a variant thereof;
(a140) a combination of tamoxifen and a protein comprising the amino acid sequence shown by SEQ ID NO:3 or a protein homologous thereto or a variant thereof;
(a141) a combination of terconazole and a protein comprising the amino acid sequence shown by SEQ ID NO:36 or a protein homologous thereto or a variant thereof;
(a142) a combination of thioproperazine and a protein comprising the amino acid sequence shown by SEQ ID NO:23 or SEQ ID NO:27 or a protein homologous thereto or a variant thereof;
(a143) a combination of thiothixene(cis) and a protein comprising the amino acid sequence shown by SEQ ID NO:23 or a protein homologous thereto or a variant thereof;
(a144) a combination of tobramycin and a protein comprising the amino acid sequence shown by SEQ ID NO:36 or a protein homologous thereto or a variant thereof;
(a145) a combination of tolbutamide and a protein comprising the amino acid sequence shown by SEQ ID NO:23 or a protein homologous thereto or a variant thereof;
(a146) a combination of trifluoperazine and a protein comprising the amino acid sequence shown by SEQ ID NO:34 or a protein homologous thereto or a variant thereof;
(a147) a combination of trimetazidine and a protein comprising the amino acid sequence shown by SEQ ID NO:5 or a protein homologous thereto or a variant thereof;
(a148) a combination of viloxazine and a protein comprising the amino acid sequence shown by SEQ ID NO:58 or a protein homologous thereto or a variant thereof;
(a149) a combination of xylazine and a protein comprising the amino acid sequence shown by SEQ ID NO:8 or a protein homologous thereto or a variant thereof;
(a150) a combination of acetylsalicylsalicylic acid and a protein comprising the amino acid sequence shown by SEQ ID NO:28 or a protein homologous thereto or a variant thereof;
(a151) a combination of nimetazepam and a protein comprising the amino acid sequence shown by SEQ ID NO:25 or a protein homologous thereto or a variant thereof;
(a152) a combination of clobazam and a protein comprising the amino acid sequence shown by SEQ ID NO:48 or a protein homologous thereto or a variant thereof;
(a153) a combination of alimemazine and a protein comprising the amino acid sequence shown by SEQ ID NO:1 or SEQ ID NO:2 or a protein homologous thereto or a variant thereof;
(a154) a combination of tranilast and a protein comprising the amino acid sequence shown by SEQ ID NO:32 or a protein homologous thereto or a variant thereof;
(a155) a combination of ebastine and a protein comprising the amino acid sequence shown by SEQ ID NO:54 or a protein homologous thereto or a variant thereof;
(a156) a combination of pranlukast and a protein comprising the amino acid sequence shown by SEQ ID NO:18, SEQ ID NO:20, SEQ ID NO:21, SEQ ID NO:35, SEQ ID NO:42, SEQ ID NO:53 or SEQ ID NO:54 or a protein homologous thereto or a variant thereof;
(a157) a combination of methyclothiazide and a protein comprising the amino acid sequence shown by SEQ ID NO:16 or SEQ ID NO:23 or a protein homologous thereto or a variant thereof;
(a158) a combination of alacepril and a protein comprising the amino acid sequence shown by SEQ ID NO:24 or a protein homologous thereto or a variant thereof;
(a159) a combination of clinofibrate and a protein comprising the amino acid sequence shown by SEQ ID NO:23 or SEQ ID NO:34 or a protein homologous thereto or a variant thereof;
(a160) a combination of acetylcysteine and a protein comprising the amino acid sequence shown by SEQ ID NO:2 or SEQ ID NO:3 or a protein homologous thereto or a variant thereof;
(a161) a combination of buformin and a protein comprising the amino acid sequence shown by SEQ ID NO:57 or a protein homologous thereto or a variant thereof;
(a162) a combination of terguride and a protein comprising the amino acid sequence shown by SEQ ID NO:9 or a protein homologous thereto or a variant thereof;
(a163) a combination of stanozolol and a protein comprising the amino acid sequence shown by SEQ ID NO:16 or a protein homologous thereto or a variant thereof;
(a164) a combination of mestanolone and a protein comprising the amino acid sequence shown by SEQ ID NO:42 or a protein homologous thereto or a variant thereof;
(a165) a combination of pantethine and a protein comprising the amino acid sequence shown by SEQ ID NO:1 or a protein homologous thereto or a variant thereof;
(a166) a combination of limaprost and a protein comprising the amino acid sequence shown by SEQ ID NO:24 or a protein homologous thereto or a variant thereof;
(a167) a combination of sarpogrelate and a protein comprising the amino acid sequence shown by SEQ ID NO:27 or a protein homologous thereto or a variant thereof;
(a168) a combination of argatroban and a protein comprising the amino acid sequence shown by SEQ ID NO:23 or a protein homologous thereto or a variant thereof;
(a169) a combination of fludroxycortide and a protein comprising the amino acid sequence shown by SEQ ID NO:25 or a protein homologous thereto or a variant thereof;
(a170) a combination of sulfadoxine and a protein comprising the amino acid sequence shown by SEQ ID NO:23 or a protein homologous thereto or a variant thereof;
(a171) a combination of ubenimex and a protein comprising the amino acid sequence shown by SEQ ID NO:23 or a protein homologous thereto or a variant thereof;
(a172) a combination of celecoxib and a protein comprising the amino acid sequence shown by SEQ ID NO:23 or a protein homologous thereto or a variant thereof;
(a173) a combination of 6-furfurylaminopurine and a protein comprising the amino acid sequence shown by SEQ ID NO:57 or a protein homologous thereto or a variant thereof;
(a174) a combination of solasodine and a protein comprising the amino acid sequence shown by SEQ ID NO:24 or a protein homologous thereto or a variant thereof;
(a175) a combination of gossypol and a protein comprising the amino acid sequence shown by SEQ ID NO:23 or a protein homologous thereto or a variant thereof;
(a176) a combination of fluorocurarine and a protein comprising the amino acid sequence shown by SEQ ID NO:10 or a protein homologous thereto or a variant thereof;
(a177) a combination of pempidine and a protein comprising the amino acid sequence shown by SEQ ID NO:57 or a protein homologous thereto or a variant thereof;
(a178) a combination of nitrarine and a protein comprising the amino acid sequence shown by SEQ ID NO:46 or SEQ ID NO:57 or a protein homologous thereto or a variant thereof;
(a179) a combination of promazine and a protein comprising the amino acid sequence shown by SEQ ID NO:18 or a protein homologous thereto or a variant thereof;
(a180) a combination of sulfabenzamide and a protein comprising the amino acid sequence shown by SEQ ID NO:23 or a protein homologous thereto or a variant thereof;
(a181) a combination of althiazide and a protein comprising the amino acid sequence shown by SEQ ID NO:23 or a protein homologous thereto or a variant thereof;
(a182) a combination of α-ergocryptine and a protein comprising the amino acid sequence shown by SEQ ID NO:23 or SEQ ID NO:53 or a protein homologous thereto or a variant thereof;
(a183) a combination of ebselen and a protein comprising the amino acid sequence shown by SEQ ID NO:6 or a protein homologous thereto or a variant thereof;
(a184) a combination of furaltadone and a protein comprising the amino acid sequence shown by SEQ ID NO:10 or a protein homologous thereto or a variant thereof;
(a185) a combination of pyrithyldione and a protein comprising the amino acid sequence shown by SEQ ID NO:55 or a protein homologous thereto or a variant thereof;
(a186) a combination of benzthiazide and a protein comprising the amino acid sequence shown by SEQ ID NO:23 or SEQ ID NO:51 or a protein homologous thereto or a variant thereof;
(a187) a combination of levobunolol and a protein comprising the amino acid sequence shown by SEQ ID NO:44 or a protein homologous thereto or a variant thereof;
(a188) a combination of raloxifene and a protein comprising the amino acid sequence shown by SEQ ID NO:37 or a protein homologous thereto or a variant thereof;
(a189) a combination of luteolin and a protein comprising the amino acid sequence shown by SEQ ID NO:20 or SEQ ID NO:54 or a protein homologous thereto or a variant thereof;
(a190) a combination of valdecoxib and a protein comprising the amino acid sequence shown by SEQ ID NO:23 or a protein homologous thereto or a variant thereof;
(a191) a combination of carboprost and a protein comprising the amino acid sequence shown by SEQ ID NO:24 or SEQ ID NO:34 or a protein homologous thereto or a variant thereof;
(a192) a combination of gabexate and a protein comprising the amino acid sequence shown by SEQ ID NO:23 or a protein homologous thereto or a variant thereof;
(b1) a combination of a protein comprising the amino acid sequence shown by SEQ ID NO:1 or a protein homologous thereto or a variant thereof and ajmaline, celestine blue, conessine, difenidol, methoxy-6-harmalan, pimethixene, quinine, ritodrine, alimemazine, boldine, clofilium, paroxetine, trimethylcolchicic acid, antipyrine, cephaeline, ciclopirox, coniine (DL), doxazosin, sulfadimethoxine, pantethine or a derivative thereof capable of binding to the protein;
(b2) a combination of a protein comprising the amino acid sequence shown by SEQ ID NO:2 or a protein homologous thereto or a variant thereof and trimethylcolchicic acid, ajmaline, celestine blue, methoxy-6-harmalan, minaprine, ritodrine, scoulerine, alimemazine, acetylcysteine or a derivative thereof capable of binding to the protein;
(b3) a combination of a protein comprising the amino acid sequence shown by SEQ ID NO:3 or a protein homologous thereto or a variant thereof and celestine blue, ciclopirox, coniine (DL), tamoxifen, acetylcysteine, paracetamol or a derivative thereof capable of binding to the protein;
(b4) a combination of a protein comprising the amino acid sequence shown by SEQ ID NO:4 or a protein homologous thereto or a variant thereof and molsidomine or a derivative thereof capable of binding to the protein;
(b5) a combination of a protein comprising the amino acid sequence shown by SEQ ID NO:5 or a protein homologous thereto or a variant thereof and trimetazidine or a derivative thereof capable of binding to the protein;
(b6) a combination of a protein comprising the amino acid sequence shown by SEQ ID NO:6 or a protein homologous thereto or a variant thereof and α-lobeline (−), ebselen or a derivative thereof capable of binding to the protein;
(b7) a combination of a protein comprising the amino acid sequence shown by SEQ ID NO:7 or a protein homologous thereto or a variant thereof and moroxydine or a derivative thereof capable of binding to the protein;
(b8) a combination of a protein comprising the amino acid sequence shown by SEQ ID NO:8 or a protein homologous thereto or a variant thereof and xylazine or a derivative thereof capable of binding to the protein;
(b9) a combination of a protein comprising the amino acid sequence shown by SEQ ID NO:9 or a protein homologous thereto or a variant thereof and terguride, iobenguane or a derivative thereof capable of binding to the protein;
(b10) a combination of a protein comprising the amino acid sequence shown by SEQ ID NO:10 or a protein homologous thereto or a variant thereof and quinine, eburnamonine, fluorocurarine, furaltadone or a derivative thereof capable of binding to the protein;
(b11) a combination of a protein comprising the amino acid sequence shown by SEQ ID NO:11 or a protein homologous thereto or a variant thereof and hydroflumethiazide or a derivative thereof capable of binding to the protein;
(b12) a combination of a protein comprising the amino acid sequence shown by SEQ ID NO:12 or a protein homologous thereto or a variant thereof and methimazole or a derivative thereof capable of binding to the protein;
(b13) a combination of a protein comprising the amino acid sequence shown by SEQ ID NO:13 or a protein homologous thereto or a variant thereof and risperidone or a derivative thereof capable of binding to the protein;
(b14) a combination of a protein comprising the amino acid sequence shown by SEQ ID NO:14 or a protein homologous thereto or a variant thereof and bupivacaine or a derivative thereof capable of binding to the protein;
(b15) a combination of a protein comprising the amino acid sequence shown by SEQ ID NO:15 or a protein homologous thereto or a variant thereof and loperamide, clofazimine, dipyridamole or a derivative thereof capable of binding to the protein;
(b16) a combination of a protein comprising the amino acid sequence shown by SEQ ID NO:16 or a protein homologous thereto or a variant thereof and stanozolol, methyclothiazide or a derivative thereof capable of binding to the protein;
(b17) a combination of a protein comprising the amino acid sequence shown by SEQ ID NO:17 or a protein homologous thereto or a variant thereof and chromomycin A3, meclofenamic acid, saquinavir or a derivative thereof capable of binding to the protein;
(b18) a combination of a protein comprising the amino acid sequence shown by SEQ ID NO:18 or a protein homologous thereto or a variant thereof and promazine, pranlukast or a derivative thereof capable of binding to the protein;
(b19) a combination of a protein comprising the amino acid sequence shown by SEQ ID NO:19 or a protein homologous thereto or a variant thereof and dihydrostreptomycin, iproniazid, nefopam or a derivative thereof capable of binding to the protein;
(b20) a combination of a protein comprising the amino acid sequence shown by SEQ ID NO:20 or a protein homologous thereto or a variant thereof and quercetin, luteolin, pranlukast or a derivative thereof capable of binding to the protein;
(b21) a combination of a protein comprising the amino acid sequence shown by SEQ ID NO:21 or a protein homologous thereto or a variant thereof and pranlukast or a derivative thereof capable of binding to the protein;
(b22) a combination of a protein comprising the amino acid sequence shown by SEQ ID NO:22 or a protein homologous thereto or a variant thereof and clemizole, fenbendazole, harmol, ifosfamide, piperlongumine, propranolol or a derivative thereof capable of binding to the protein;
(b23) a combination of a protein comprising the amino acid sequence shown by SEQ ID NO:23 or a protein homologous thereto or a variant thereof and acetohexamide, benzethonium, clomiphene, cyclobenzaprine, flupentixol, guanfacine, maprotiline, perhexiline, probenecid, clinofibrate, celecoxib, gossypol, althiazide, α-ergocryptine, gabexate, clenbuterol, etodolac, misoprostol, ubenimex, clopamide, glibenclamide, glipizide, isoxicam, sulfaphenazole, thioproperazine, thiothixene(cis), tolbutamide, methyclothiazide, argatroban, sulfadoxine, sulfabenzamide, benzthiazide, valdecoxib or a derivative thereof capable of binding to the protein;
(b24) a combination of a protein comprising the amino acid sequence shown by SEQ ID NO:24 or a protein homologous thereto or a variant thereof and acetohexamide, isradipine, mupirocin, limaprost, solasodine, alacepril, carboprost or a derivative thereof capable of binding to the protein;
(b25) a combination of a protein comprising the amino acid sequence shown by SEQ ID NO:25 or a protein homologous thereto or a variant thereof and metergotamine, methoxamine, paroxetine, dizocilpine, fluvoxamine, 3-hydroxykynurenine, nimetazepam, fludroxycortide or a derivative thereof capable of binding to the protein;
(b26) a combination of a protein comprising the amino acid sequence shown by SEQ ID NO:26 or a protein homologous thereto or a variant thereof and fenoprofen or a derivative thereof capable of binding to the protein;
(b27) a combination of a protein comprising the amino acid sequence shown by SEQ ID NO:27 or a protein homologous thereto or a variant thereof and acenocoumarol, budesonide, chlorogenic acid, chlorothiazide, diclofenac, diperodon, DO897/99, nimesulide, thioproperazine, sarpogrelate or a derivative thereof capable of binding to the protein;
(b28) a combination of a protein comprising the amino acid sequence shown by SEQ ID NO:28 or a protein homologous thereto or a variant thereof and acetylsalicylsalicylic acid or a derivative thereof capable of binding to the protein;
(b29) a combination of a protein comprising the amino acid sequence shown by SEQ ID NO:29 or a protein homologous thereto or a variant thereof and buspirone or a derivative thereof capable of binding to the protein;
(b30) a combination of a protein comprising the amino acid sequence shown by SEQ ID NO:30 or a protein homologous thereto or a variant thereof and dopamine, α-methyl-5-hydroxytryptamine or a derivative thereof capable of binding to the protein;
(b31) a combination of a protein comprising the amino acid sequence shown by SEQ ID NO:31 or a protein homologous thereto or a variant thereof and cisapride or a derivative thereof capable of binding to the protein;
(b32) a combination of a protein comprising the amino acid sequence shown by SEQ ID NO:32 or a protein homologous thereto or a variant thereof and berberine, celestine blue, diflunisal, mebendazole, tranilast or a derivative thereof capable of binding to the protein;
(b33) a combination of a protein comprising the amino acid sequence shown by SEQ ID NO:33 or a protein homologous thereto or a variant thereof and bromperidol, coralyne or a derivative thereof capable of binding to the protein;
(b34) a combination of a protein comprising the amino acid sequence shown by SEQ ID NO:34 or a protein homologous thereto or a variant thereof and DO897/99, domperidone, flupentixol, fluphenazine, L-thyroxine, trifluoperazine, clinofibrate, acetohexamide, chromomycin A3, carboprost or a derivative thereof capable of binding to the protein;
(b35) a combination of a protein comprising the amino acid sequence shown by SEQ ID NO:35 or a protein homologous thereto or a variant thereof and alfuzosin, clobetasone, doxazosin, pranlukast, risperidone or a derivative thereof capable of binding to the protein;
(b36) a combination of a protein comprising the amino acid sequence shown by SEQ ID NO:36 or a protein homologous thereto or a variant thereof and acetopromazine, cyclopentolate, perhexiline, phenformin, pyrilamine, terconazole, tobramycin, amoxapine, cephaeline, clenbuterol, domperidone, minocycline, moxalactam or a derivative thereof capable of binding to the protein;
(b37) a combination of a protein comprising the amino acid sequence shown by SEQ ID NO:37 or a protein homologous thereto or a variant thereof and glibenclamide, raloxifene, clofazimine or a derivative thereof capable of binding to the protein;
(b38) a combination of a protein comprising the amino acid sequence shown by SEQ ID NO:38 or a protein homologous thereto or a variant thereof and albendazole or a derivative thereof capable of binding to the protein;
(b39) a combination of a protein comprising the amino acid sequence shown by SEQ ID NO:39 or a protein homologous thereto or a variant thereof and bezafibrate or a derivative thereof capable of binding to the protein;
(b40) a combination of a protein comprising the amino acid sequence shown by SEQ ID NO:40 or a protein homologous thereto or a variant thereof and pirenzepine or a derivative thereof capable of binding to the protein;
(b41) a combination of a protein comprising the amino acid sequence shown by SEQ ID NO:41 or a protein homologous thereto or a variant thereof and rescinnamine or a derivative thereof capable of binding to the protein;
(b42) a combination of a protein comprising the amino acid sequence shown by SEQ ID NO:42 or a protein homologous thereto or a variant thereof and benzbromarone, pranlukast, mifepristone, mestanolone or a derivative thereof capable of binding to the protein;
(b43) a combination of a protein comprising the amino acid sequence shown by SEQ ID NO:43 or a protein homologous thereto or a variant thereof and hydroxytacrine(R,S), metergotamine, metaproterenol or a derivative thereof capable of binding to the protein;
(b44) a combination of a protein comprising the amino acid sequence shown by SEQ ID NO:44 or a protein homologous thereto or a variant thereof and ebumamonine, levobunolol or a derivative thereof capable of binding to the protein;
(b45) a combination of a protein comprising the amino acid sequence shown by SEQ ID NO:45 or a protein homologous thereto or a variant thereof and norharman, pyrilamine or a derivative thereof capable of binding to the protein;
(b46) a combination of a protein comprising the amino acid sequence shown by SEQ ID NO:46 or a protein homologous thereto or a variant thereof and celestine blue, nitrarine or a derivative thereof capable of binding to the protein;
(b47) a combination of a protein comprising the amino acid sequence shown by SEQ ID NO:47 or a protein homologous thereto or a variant thereof and clemizole or a derivative thereof capable of binding to the protein;
(b48) a combination of a protein comprising the amino acid sequence shown by SEQ ID NO:48 or a protein homologous thereto or a variant thereof and clobazam or a derivative thereof capable of binding to the protein;
(b49) a combination of a protein comprising the amino acid sequence shown by SEQ ID NO:49 or a protein homologous thereto or a variant thereof and josamycin, oxytocin, clarithromycin or a derivative thereof capable of binding to the protein;
(b50) a combination of a protein comprising the amino acid sequence shown by SEQ ID NO:50 or a protein homologous thereto or a variant thereof and leuprolide, cyclosporine A or a derivative thereof capable of binding to the protein;
(b51) a combination of a protein comprising the amino acid sequence shown by SEQ ID NO:51 or a protein homologous thereto or a variant thereof and dichlorphenamide, benzthiazide or a derivative thereof capable of binding to the protein;
(b52) a combination of a protein comprising the amino acid sequence shown by SEQ ID NO:52 or a protein homologous thereto or a variant thereof and hydroxychloroquine, furazolidone, metanephrine (D,L) or a derivative thereof capable of binding to the protein;
(b53) a combination of a protein comprising the amino acid sequence shown by SEQ ID NO:53 or a protein homologous thereto or a variant thereof and benzbromarone, benzethonium, clofazimine, domperidone, doxazosin, gramicidin, α-ergocryptine, bicartamide, rescinnamine, saquinavir, syrosingopine, pranlukast or a derivative thereof capable of binding to the protein;
(b54) a combination of a protein comprising the amino acid sequence shown by SEQ ID NO:54 or a protein homologous thereto or a variant thereof and benzbromarone, clofazimine, domperidone, nicardipine, quercetin, ebastine, actinomycin D, loperamide, pranlukast, luteolin or a derivative thereof capable of binding to the protein;
(b55) a combination of a protein comprising the amino acid sequence shown by SEQ ID NO:55 or a protein homologous thereto or a variant thereof and pyrithyldione or a derivative thereof capable of binding to the protein;
(b56) a combination of a protein comprising the amino acid sequence shown by SEQ ID NO:56 or a protein homologous thereto or a variant thereof and chlordiazepoxide, flumequine or a derivative thereof capable of binding to the protein;
(b57) a combination of a protein comprising the amino acid sequence shown by SEQ ID NO:57 or a protein homologous thereto or a variant thereof and buformin, 6-furfurylaminopurine, nitrarine, pempidine or a derivative thereof capable of binding to the protein;
(b58) a combination of a protein comprising the amino acid sequence shown by SEQ ID NO:58 or a protein homologous thereto or a variant thereof and viloxazine or a derivative thereof capable of binding to the protein;
(b59) a combination of a protein comprising the amino acid sequence shown by SEQ ID NO:59 or a protein homologous thereto or a variant thereof and cefazolin, fenbufen, ketoprofen, colchicine, doxycycline, gabapentin, lidoflazine, probenecid or a derivative thereof capable of binding to the protein;
(b60) a combination of a protein comprising the amino acid sequence shown by SEQ ID NO:60 or a protein homologous thereto or a variant thereof and benzydamine, clenbuterol or a derivative thereof capable of binding to the protein;
(b61) a combination of a protein comprising the amino acid sequence shown by SEQ ID NO:61 or a protein homologous thereto or a variant thereof and benzethonium, fluphenazine, GBR12909, doxazosin, procaine, quinacrine or a derivative thereof capable of binding to the protein;
(b62) a combination of a protein comprising the amino acid sequence shown by SEQ ID NO:62 or a protein homologous thereto or a variant thereof and azithromycin, colistin or a derivative thereof capable of binding to the protein;
(b63) a combination of a protein comprising the amino acid sequence shown by SEQ ID NO:63 or a protein homologous thereto or a variant thereof and protriptyline, maprotiline or a derivative thereof capable of binding to the protein.

19. A method of producing the complex according to claim 18, which comprises bringing the bioactive substance and the target protein therefor into contact with each other.

20. A kit comprising the following (i) and (ii):
(i) a bioactive substance X or a salt thereof;
(ii) a target protein Y, a nucleic acid that encodes the protein, an expression vector comprising the nucleic acid, cells that enable a measurement of the expression of the target protein Y or a gene that encodes the protein, or an expression vector comprising the transcription regulatory region of a gene that encodes the target protein Y and a reporter gene functionally linked thereto;
wherein the combination of the bioactive substance X and the target protein Y is any of (a1) to (a192) or (b1) to (b63) of claim 18.

21. A method for determining the onset or risk of onset of a disease or condition associated with an action of a bioactive substance X, or a disease or condition associated with a function of a target protein Y, which comprises the following steps (a) and (b):
(a) a step for measuring the expression level and/or polymorphism of the target protein Y or a gene that encodes the protein in a biological sample collected from an animal;
(b) a step for evaluating the onset or likelihood of onset of the disease or condition on the basis of the measured expression level and/or polymorphism;
wherein the combination of the bioactive substance X and the target protein Y is any of (a1) to (a192) or (b1) to (b63) of claim 18.

22. A kit for determining the onset or risk of onset of a disease or condition associated with an action of a bioactive substance X, or a disease or condition associated with a function of a target protein Y, which comprises the following (i) and (ii):
(i) a means capable of measuring the expression level and/or polymorphism of the target protein Y or a gene that encodes the protein;
(ii) a medium recording the relationship between the disease or condition and the expression level and/or polymorphism of the gene;
wherein the combination of the bioactive substance X and the target protein Y is any of (a1) to (a192) or (b1) to (b63) of claim 18.

23. A method for determining susceptibility to a bioactive substance X in a disease or condition associated with an action of the bioactive substance X, or a disease or condition associated with a function of a target protein Y, which comprises the following steps (a) and (b):
(a) a step for measuring the expression level and/or polymorphism of the target protein Y or a gene that encodes the protein in a biological sample collected from an animal;
(b) a step for predicting the effect of the bioactive substance on the basis of the measured expression level and/or polymorphism;
wherein the combination of the bioactive substance X and the target protein Y is any of (a1) to (a192) or (b1) to (b63) of claim 18.

24. A kit for determining susceptibility to a bioactive substance X in a disease or condition associated with an action of the bioactive substance X, or a disease or condition associated with a function of a target protein Y, which comprises the following steps (a) and (b):
(i) a means capable of measuring the expression level and/or polymorphism of the target protein Y or a gene that encodes the protein;
(ii) a medium recording the relationship between the effect of the bioactive substance X and said expression level and/or polymorphism of the gene;
wherein the combination of the bioactive substance X and the target protein Y is any of (a1) to (a192) or (b1) to (b63) of claim 18.

25. A polynucleotide of any of the following (a) to (d):
(a) a polynucleotide consisting of the nucleotide sequence shown by SEQ ID NO: 64;
(b) a polynucleotide consisting of the nucleotide sequence shown by SEQ ID NO: 65;
(c) a polynucleotide consisting of a nucleotide sequence corresponding to the 606th-2363rd nucleotides of the nucleotide sequence shown by SEQ ID NO: 64; and
(d) a polynucleotide consisting of a nucleotide sequence corresponding to the 571st-1485th nucleotides of the nucleotide sequence shown by SEQ ID NO: 65.
